(19) United States
(12) Patent Application Publication
(10) Pub. No.: US 2023/0241241 A1
(43) Pub. Date: Aug. 3, 2023

(54) CONJUGATES OF A CELL-BINDING MOLECULE WITH CAMPTOTHECIN ANALOGS

(71) Applicant: HANGZHOU DAC BIOTECH CO., LTD., Hangzhou, Zhejiang (CN)

(72) Inventors: Robert ZHAO, Lexington, MA (US); Qingliang YANG, Hangzhou, Zhejiang (CN); Hangbo YE, Hangzhou, Zhejiang (CN); Yuanyuan HUANG, Hangzhou, Zhejiang (CN); Yifang XU, Hangzhou, Zhejiang (CN); Gengxiang ZHAO, Hangzhou, Zhejiang (CN); Diancheng CHEN, Hangzhou, Zhejiang (CN); Huihui GUO, Hangzhou, Zhejiang (CN); Xiangfei KONG, Hangzhou, Zhejiang (CN); Wenjun LI, Hangzhou, Zhejiang (CN); Lu BAI, Hangzhou, Zhejiang (CN); Xiang CAI, Hangzhou, Zhejiang (CN); Xiuzhen ZHANG, Hangzhou, Zhejiang (CN); Junxiang JIA, Hangzhou, Zhejiang (CN); Zhixiang GUO, Hangzhou, Zhejiang (CN); Shangma HUANG, Hangzhou, Zhejiang (CN); Xiaoxu WANG, Hangzhou, Zhejiang (CN); Jun ZHENG, Hangzhou, Zhejiang (CN); Yong DU, Hangzhou, Zhejiang (CN); Yanhua LI, Hangzhou, Zhejiang (CN); Yunxia ZHENG, Hangzhou, Zhejiang (CN); Chen LIN, Hangzhou, Zhejiang (CN); Xiaoxiao CHEN, Hangzhou, Zhejiang (CN); Wei ZHENG, Hangzhou, Zhejiang (CN); Xinyan JIANG, Hangzhou, Zhejiang (CN); Lingli ZHANG, Hangzhou, Zhejiang (CN); Riping YE, Hangzhou, Zhejiang (CN); Miaomiao CHEN, Hangzhou, Zhejiang (CN)

(73) Assignee: HANGZHOU DAC BIOTECH CO., LTD., Hangzhou, Zhejiang (CN)

(21) Appl. No.: 18/001,899

(22) PCT Filed: Jun. 19, 2020
(86) PCT No.: PCT/CN2020/097239

§ 371 Date: Dec. 15, 2022

(51) Int. Cl. A61K 47/68; A61P 35/00

(52) U.S. Cl. CPC A61K 47/6849; A61K 47/6803; A61P 35/00; A61K 47/6851

(57) ABSTRACT

This invention relates to conjugates of camptothecin analogs with a cell-binding molecule of formula (I), wherein R¹, R², R³, R⁴, R⁵, X, L, n, m, T and ----- are defined herein. It also provides methods of making the conjugates of camptothecin analogs to a cell-binding agent, as well as methods of using the conjugates in targeted treatment of cancer, infection, and immunological disorders.

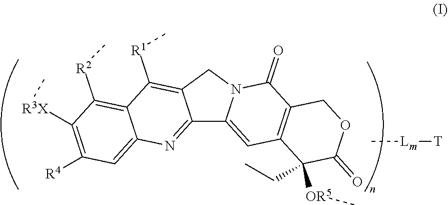

### FIELD OF THE INVENTION

This invention relates to conjugates of a camptothecin analog with a cell-surface receptor-binding molecule for targeted therapy. The invention also relates to use of compositions comprising a conjugate of the camptothecin analog to a cell-binding molecule for targeted treatment of cancer, autoimmune disease, and infectious disease.

### BACKGROUND OF THE INVENTION

Targeted cancer strategies aim to minimize or overcome such side effects by better targeting the tumor and avoiding healthy tissues. One of the strategies is antibody-drug conjugate (ADC) which combines the precision of the antibody towards the tumor with the high potent cytotoxicity of the drug in question (the payload) through a conditionally stable linker, thereby increasing the local concentration of the latter several-folds than the health tissues. The intensive research and the huge funding by the pharmaceutical industries on ADCs during the past four decades have led to USFDA approval of 8 ADCs, named Mylotarg (gemtuzumab ozogamicin), Adcetris (brentuximab vedotin), Kadcyla (ado-trastuzumab emtansine), Besponsa (inotuzumab ozogamicin), Polivy (polatuzumab vedotin-piiq), Enhertu (fam-trastuzumab deruxtecan-nxki), Padcev (enfortumab vedotin-ejfv), Trodelvy (sacituzumab govitecan) and over 100 ADC drugs are now in the clinic development (Chau, C. H. et al, Lancet. 2019, 394, 793-804).

It has been known that the payload-linker component in the ADC complexes modality critically contribute to ADC homogeneity, circulation stability, pharmacokinetic profiles, tolerability, and overall treatment efficacy (Zhao, R. Y. et al (2011) J. Med. Chem. 54, 3606; Acchionea, M. et al (2012) mAbs, 4, 362; Doronina, S. et al, (2006) Bioconjug Chem, 17, 114; Hamann, P. et al. (2005) Bioconjug Chem. 16, 346). Despite extensive study to improve these parameters for the next generation of ADCs, most payloads used to date are still narrowly selected from maytansins (DM1 and DM4), auruistatins (MMAE and MMAF), calicheamicins, Pyrrolo[2,1-c][1,4]benzodiazepine (PBD) dimers, camptothecins, duocarmysins and tubulysins (Leung, D., et al, Antibodies (Basel). 2020, 9: E2. doi: 10.3390/antib9010002; Khongorzul, P., et al, Mol Cancer Res. 2020, 18: 3-19. doi: 10.1158/1541-7786.MCR-19-0582; Chau, C. H., et al, Lancet. 2019, 394:793-804. doi: 10.1016/S0140-6736(19)31774-X).

Among these payloads, the camptothecins (CPTs) have proved promising choice with a wider therapeutic index (TI) than many other payloads for ADC construction as two of their ADC compounds, Enhertu (fam-trastuzumab deruxtecan-nxki, or DS-8201a) and Sacituzumab govitecan (IMMU-132 or hRS7-SN-38) have demonstrated significant clinical benefits (PFS and OS) for solid tumors in many clinical trials (Pondé, N., et al, Curr Treat Options Oncol. 2019 Apr. 1; 20(5):37. doi: 10.1007/s11864-019-0633-6; Kaplon, H., et al, MAbs. 2020, 12(1): 1703531, 10.1080/19420862.2019.1703531). Camptothecin (CPT) is a potent antitumor antibiotic isolated in 1958 from extracts of Camptotheca acuminata, a tree native to China wherein the plant has been extensively used in traditional Chinese medicine for hundreds of years. Camptothecin can cause a cell death through interacting with DNA enzyme topoisomerase I and then accumulating reversible enzyme-camptothecin-DNA ternary complexes (Wu Du, Tetrahedron 59 (2003) 8649-8687). Many of camptothecin analogs have been disclosed during the past five decades as shown below:

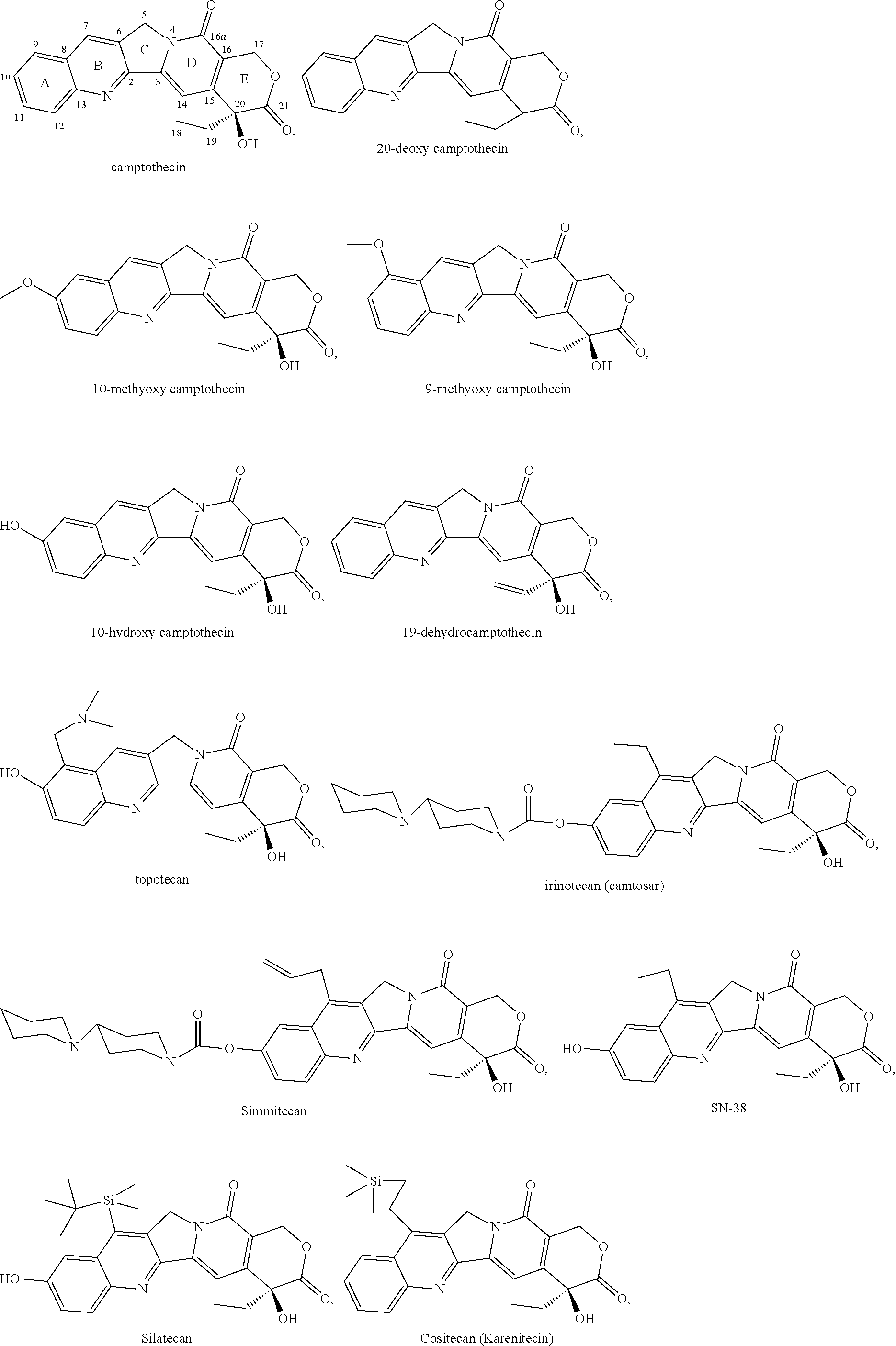

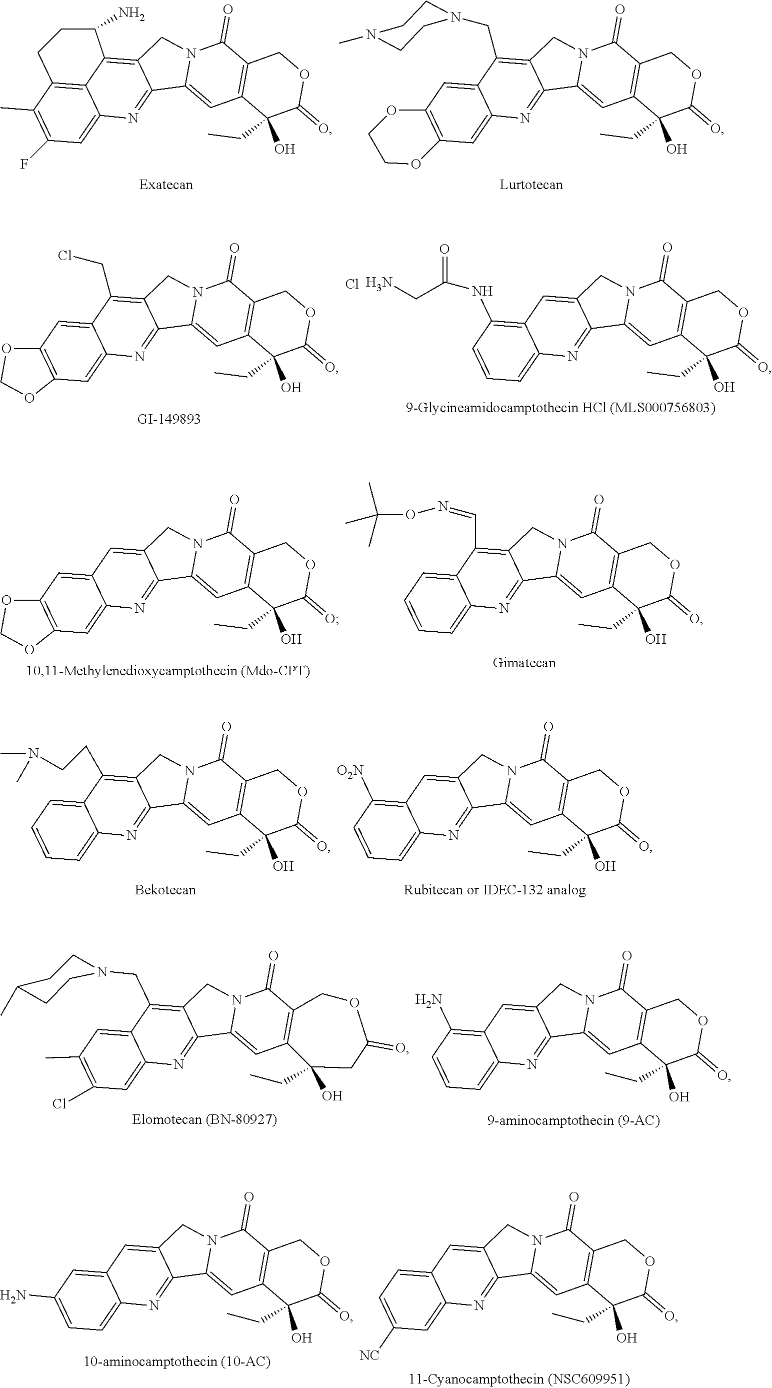

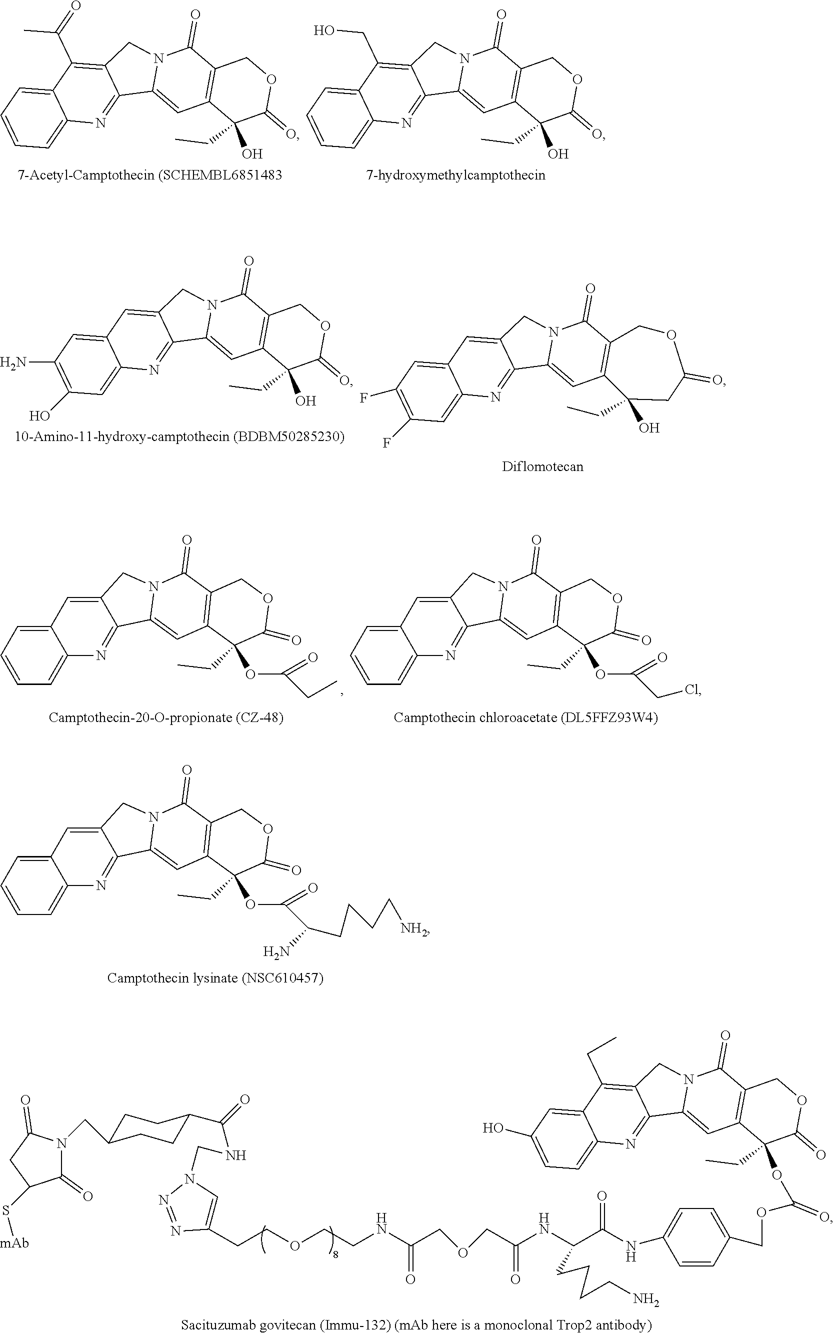

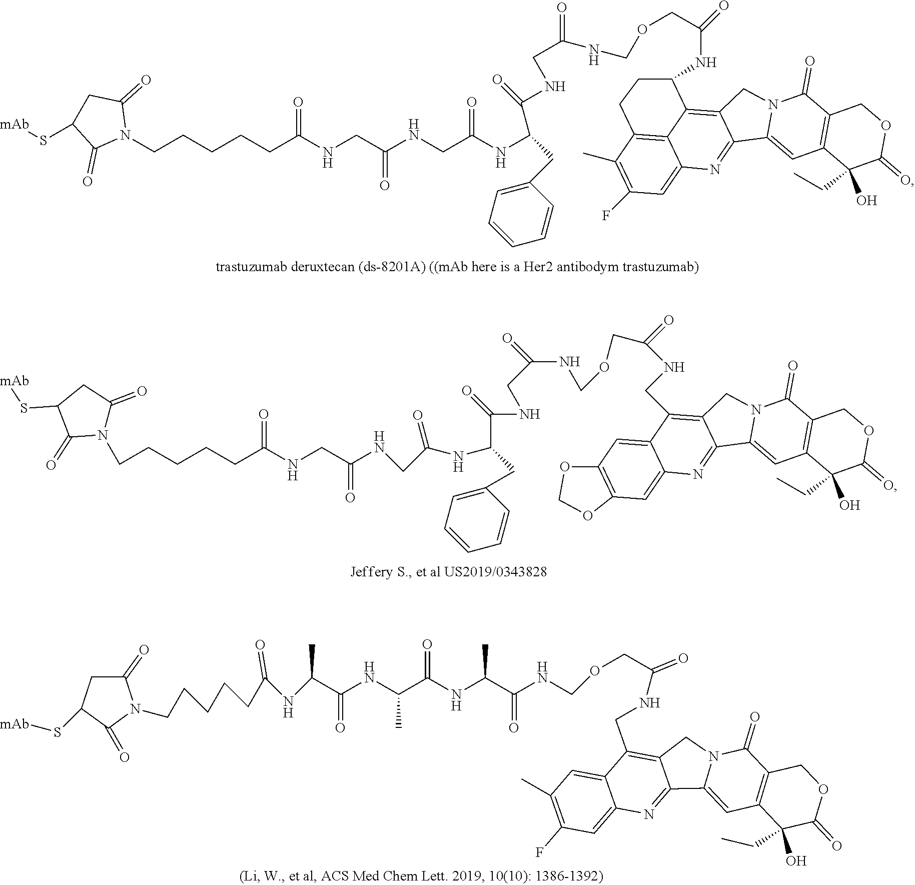

Camptothecin (CPT) and most of its analogs are extremely insoluble in physiological buffer and high adverse drug reaction in the preliminary clinical trials since 1970s. Camptothecin ADC cause their ADC conjugates aggregation up to 80% (Burke, P., et al Bioconjugate Chem. 2009, 20, 6, 1242-1250) that can limit the successes of the scale-up manufacturing production and the attainments of clinical trials due to systemic side-effects resulting from the aggregation. So far US FDA only approved three water-soluble CPT analogues, topotecan, irinotecan, and belotecan that are used in cancer chemotherapy (Palakurthi, S., Expert Opin Drug Deliv. 2015; 12(12):1911-21; Shang, X. F. et al, Med Res Rev. 2018, 38(3):775-828) and one water-soluble CPT analog ADC, Enhertu (fam-trastuzumab deruxtecan-nxki, or DS-8201a) as targeted immunotherapy for Her2 solid tumor (Modi S, et al, N Engl J Med. 2020, 382(7): 610-621. doi: 10.1056/NEJMoa1914510; Keam, S. J., Drugs. 2020 April; 80(5):501-508. doi: 10.1007/s40265-020-01281-4). We have worked on water-soluble CPT analog ADC for quite a while and applied hydrophilic side-chain linkers to the CPT analog ADCs (see PCT/CN2019/092614) to widen the therapeutic windows of the ADCs. Here this application disclosed a water-soluble CPT analog ADC wherein C-10 position of the CPT analog that is linked to O or NH is critical for water solubility and C-11 position linked to an electron withdrawing group or a bulking group to maintain highly potent cytotoxity as in comparison to natural CPTs.

### SUMMARY OF THE INVENTION

The invention provides camptothecin analog conjugates to a cell-binding molecule, camptothecin analog-linker compounds and camptothecin analog compounds, methods of preparing and using them, and intermediates useful in the preparation thereof. The camptothecin analog conjugates of the present invention are water-soluble and stable in blood circulation, as well capable of causing cell death once free camptothecin analog compound or a metabolite of camptothecin analog-linker compound is released from the conjugate in the vicinity or within disordered cells.

In one illustrative embodiment of the invention provides a conjugate of camptothecin analogs of Formula (I):

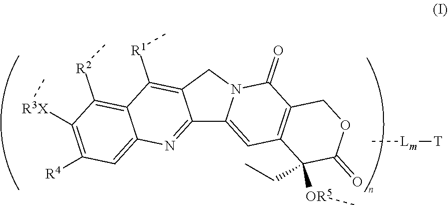

or their pharmaceutically acceptable salts, hydrates, or hydrated salts; or the polymorphic crystalline structures of these compounds; or their isotopes, optical isomers, racemates, diastereomers or enantiomers thereof;

Wherein T is a targeting or binding ligand; L is a releasable linker; ----- is a linkage bond that L connects to an atom of R¹, R², R³ or R⁵ independently inside the bracket independently; n is 1-30 and m is 1-10;

Inside the bracket is a potent camptothecin analog wherein:

R¹ and R² are independently H; linear or branched C₁-C₆ of alkyl, alkyl alcohol, alkyl amine (including primary, secondary, tertiary amine, or quaternary ammonium), aminoalkyl, oxylalkyl, aminoalkylamino, oxylalkylamino, aminoalkyloxyl, oxylalkyloxyl, alkyl carboxylic acid; C₂-C₆ of heteroalkyl, alkylcycloalkyl, heterocycloalkyl, heterocyclic, aminocycloalkyl, heteroalkylcycloalkyl, alkylcarbonyl, alkyl ether, alkyl ester, alkyl amide, oxylalkylamide, aminoalkylamide, oxime; NH₂, or OH;

R³ is independently H, C(O)NH, C(O)O, SO₂R⁶, SO₃R⁶, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR^{6′})₂, C(O)R⁶, C(O)NHR⁶; linear or branched C₁-C₆ of alkyl, alkyl alcohol, alkyl amine (including primary, secondary, tertiary amine, or quaternary ammonium), or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide, oxime; C₅˜C₁₂ glycoside, NH₂, or OH;

R⁴ is halo (F, Cl, Br, or I), CN, NO₂, SO₃H, OR⁶, SR⁶, S(O₂)R⁶, NHR⁶, N(R⁶)(R^{6′}), C(O)XR⁶, N+(R⁶)(R^{6′})(R^{6″});

X is NH or O;

R⁵ is H, C(O)O, C(O)NH, R⁶C(O), linear or branched C₁-C₆ of alkyl, alkyl alcohol, alkyl amine (including primary, secondary, tertiary amine, or quaternary ammonium), alkyl carboxylic acid; C₂-C₆ of carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;

R⁶, R^{6′}, and R^{6″} are independently C₁-C₆ of alkyl, alkyl alcohol, alkyl amine (including primary, secondary, tertiary amine, or quaternary ammonium) or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or pharmaceutical salts; In addition, R¹, R², R³, and R⁶ can be independently absent, and R², R³, X, C-9, and C-10 can link together to form a 5, 6 or 7-member heterocyclic ring.

In another embodiment, the linker L of the potent Camptothecin analog-binding molecule conjugates has the formula: --Ww-(Aa)r--Vv-; wherein: --W-- is a Stretcher unit; w is 0 or 1; each --Aa-- is independently an Amino Acid unit; r is independently an integer ranging from 0 to 12; --V-- is a Spacer unit; and v is 0, 1 or 2. The Stretcher unit W may independently contain a self-immolative spacer, peptidyl units, a hydrazone bond, disulfide or thioether bonds.

In another embodiment, the cell-surface binding molecule T may be of any kind presently known, or which become known cell binding ligands, such as peptides and non-peptides. Generally the cell-binding molecule T is an antibody; a single chain antibody; an antibody fragment that binds to the target cell; a monoclonal antibody; a single chain monoclonal antibody; or a monoclonal antibody fragment that binds the target cell; a chimeric antibody; a chimeric antibody fragment that binds to the target cell; a domain antibody; a domain antibody fragment that binds to the target cell; adnectins that mimic antibodies; DARPins; a lymphokine; a hormone; a vitamin; a growth factor; a colony stimulating factor; or a nutrient-transport molecule (a transferrin); a binding peptide, or protein, or antibody, or small affinity molecule attached on albumin, polymers, dendrimers, liposomes, nanoparticles, vesicles, (viral) capsids. Preferably the binding molecule T is a monoclonal antibody.

In yet another aspect, a compound of formula (I) or a pharmaceutically acceptable salt or solvate thereof is used for treating cancer, an autoimmune disease or an infectious disease in a human or an animal.

### BRIEF DESCRIPTION OF THE DRAWINGS

FIG. 1 shows the synthesis of camptothecin analogs containing a conjugatable linker.

FIG. 2 shows the synthesis of camptothecin analogs containing a conjugatable linker.

FIG. 3 shows the synthesis of camptothecin analogs containing a conjugatable linker.

FIG. 4 shows the synthesis of camptothecin analogs containing a conjugatable linker.

FIG. 5 shows the synthesis of camptothecin analogs containing a conjugatable linker.

FIG. 6 shows the synthesis of camptothecin analogs containing a conjugatable linker.

FIG. 7 shows the synthesis of camptothecin analogs containing a conjugatable linker.

FIG. 8 shows the synthesis of camptothecin analogs containing a conjugatable linker.

FIG. 9 shows the synthesis of camptothecin analogs containing a conjugatable linker.

FIG. 10 shows the synthesis of camptothecin analogs containing a conjugatable linker.

FIG. 11 shows the synthesis of camptothecin analogs containing a conjugatable linker.

FIG. 12 shows the synthesis of camptothecin analogs containing a conjugatable linker.

FIG. 13 shows the synthesis of camptothecin analogs containing a conjugatable linker.

FIG. 14 shows the synthesis of camptothecin analogs containing a conjugatable linker.

FIG. 15 shows the synthesis of camptothecin analogs containing a conjugatable linker.

FIG. 16 shows the synthesis of camptothecin analogs containing a conjugatable linker.

FIG. 17 shows the synthesis of camptothecin analogs containing a conjugatable linker.

FIG. 18 shows the synthesis of camptothecin analogs containing a conjugatable linker.

FIG. 19 shows the synthesis of a camptothecin analog containing a conjugatable linker and its conjugate to an antibody.

FIG. 20 shows the synthesis of a camptothecin analog containing a conjugatable linker and its conjugate to an antibody.

FIG. 21 shows the synthesis of camptothecin analogs containing a conjugatable linker.

FIG. 22 shows the synthesis of a camptothecin analog containing a conjugatable linker and its conjugate to an antibody.

FIG. 23 shows the synthesis of a camptothecin analog containing a conjugatable linker and its conjugate to an antibody.

FIG. 24 shows the synthesis of a camptothecin analog containing a conjugatable linker and its conjugate to an antibody.

FIG. 25 shows the synthesis of a camptothecin analog containing a conjugatable linker and its conjugate to an antibody.

FIG. 26 shows the synthesis of a camptothecin analog containing a conjugatable linker and its conjugate to an antibody.

FIG. 27 shows the synthesis of a camptothecin analog containing a conjugatable linker.

FIG. 28 shows the synthesis of a conjugatable linker for conjugates of camptothecin analogs.

FIG. 29-1 shows the synthesis of a camptothecin analog containing a conjugatable linker.

FIG. 29-2 shows structures of camptothecin analogs for used in the conjugates.

FIG. 30 shows the synthesis of a camptothecin analog containing a conjugatable linker.

FIG. 31 shows the synthesis of a camptothecin analog containing a conjugatable linker.

FIG. 32 shows the synthesis of a camptothecin analog containing a conjugatable linker.

FIG. 33 shows structures of the conjugates of antibody-camptothecin analogs.

FIG. 34 shows the comparison of anti-tumor effect in vivo of Her2 antibody-CPT analog conjugates C1-031, C1-238, C1-397, C1-407, C1-411, C1-414, C1-424, C1-428 with T-DM1 using human gastric tumor N87 cell model at dose of 6 mg/Kg, i.v.

FIG. 35 shows the in vivo toxicity study of the Her2 antibody-CPT analog conjugates C₁-031, C1-226, C1-238, C1-397, C1-407, C1-411, C1-414, C1-424, C1-428 in comparison with T-DM1 at dose of 150 mg/Kg, i.v.

FIG. 36 shows the anti-tumor effect in vivo of EGFR antibody-CPT analog conjugates C1-031, C1-200, C1-214, C1-226, C1-305, C1-306, C1-311, C1-362, C1-402, C-407 and C1-419 using human NSCLC tumor HCC827 cell model at dose of 6 mg/Kg, i.v.

### DETAILED DESCRIPTION OF THE INVENTION

### Definitions

“Alkyl” refers to an aliphatic hydrocarbon group or univalent groups derived from alkane by removal of one or two hydrogen atoms from carbon atoms. It may be straight or branched having C₁-C₈ (1 to 8 carbon atoms) in the chain. “Branched” means that one or more lower C numbers of alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl, cyclopentyl, cyclohexyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methyl-hexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)₂R′, —S(O)R′, —OH, -halogen, —N₃, —NH₂, —NH(R′), —N(R′)₂ and —CN; where each R′ is independently selected from —C₁-C₈ alkyl and aryl.

“Halogen” refers to fluorine, chlorine, bromine or iodine atom; preferably fluorine and chlorine atom.

“Heteroalkyl” refers to C₂-C₈ alkyl in which one to four carbon atoms are independently replaced with a heteroatom from the group consisting of O, S and N.

“Carbocycle” refers to a saturated or unsaturated ring having 3 to 8 carbon atoms as a monocycle or 7 to 13 carbon atoms as a bicycle. Monocyclic carbocycles have 3 to 6 ring atoms, more typically 5 or 6 ring atoms. Bicyclic carbocycles have 7 to 12 ring atoms, arranged as a bicycle [4,5], [5,5], [5,6] or [6,6] system, or 9 or 10 ring atoms arranged as a bicycle [5,6] or [6,6] system. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, —1,4-cyclohexadienyl, -cycloheptyl, -1,3-cycloheptadienyl, -1,3,5-cycloheptatrienyl, -cyclooctyl, and -cyclooctadienyl.

A “C₃-C₈ carbocycle” refers to a 3-, 4-, 5-, 6-, 7- or 8-membered saturated or unsaturated nonaromatic carbocyclic ring. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)R′, —S(O)₂R′, —OH, -halogen, —N₃, —NH₂, —NH(R′), —N(R′)₂ and —CN; where each R′ is independently selected from —C₁-C₈ alkyl and aryl.

“Alkenyl” refers to an aliphatic hydrocarbon group containing a carbon-carbon double bond which may be straight or branched having 2 to 8 carbon atoms in the chain. Exemplary alkenyl groups include ethenyl, propenyl, n-butenyl, i-butenyl, 3-methylbut-2-enyl, n-pentenyl, hexylenyl, heptenyl, octenyl.

“Alkynyl” refers to an aliphatic hydrocarbon group containing a carbon-carbon triple bond which may be straight or branched having 2 to 8 carbon atoms in the chain. Exemplary alkynyl groups include ethynyl, propynyl, n-butynyl, 2-butynyl, 3-methylbutynyl, 5-pentynyl, n-pentynyl, hexynyl, heptenyl, and octynyl.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene, propargyl and 4-pentynyl.

“Aryl” or Ar refers to an aromatic or hetero aromatic group, composed of one or several rings, comprising three to fourteen carbon atoms, preferentially six to ten carbon atoms. The term of “hetero aromatic group” refers one or several carbon on aromatic group, preferentially one, two, three or four carbon atoms are replaced by O, N, Si, Se, P or S, preferentially by O, S, and N. The term aryl or Ar also refers to an aromatic group, wherein one or several H atoms are replaced independently by —R′, -halogen, —OR′, or —SR′, —NR′R″, —N═NR′, —N═R′, —NR′R″, —NO₂, —S(O)R′, —S(O)₂R′, —S(O)₂OR′, —OS(O)₂OR′, —PR′R″, —P(O)R′R″, —P(OR′)(OR″), —P(O)(OR′)(OR″) or —OP(O)(OR′)(OR″) wherein R′, R″ are independently H, alkyl, alkenyl, alkynyl, heteroalkyl, aryl, arylalkyl, carbonyl, or pharmaceutical salts.

“Heterocycle” refers to a ring system in which one to four of the ring carbon atoms are independently replaced with a heteroatom from the group of O, N, S, Se, B, Si and P. Preferable heteroatoms are O, N and S. Heterocycles are also described in The Handbook of Chemistry and Physics, 78th Edition, CRC Press, Inc., 1997-1998, p. 225 to 226, the disclosure of which is hereby incorporated by reference. Preferred nonaromatic heterocyclic include epoxy, aziridinyl, thiiranyl, pyrrolidinyl, pyrazolidinyl, imidazolidinyl, oxiranyl, tetrahydrofuranyl, dioxolanyl, tetrahydropyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydropyranyl, tetrahydropyranyl, dihydropyranyl, tetrahydropyridyl, dihydropyridyl, tetrahydropyrimidinyl, dihydrothiopyranyl, azepanyl, as well as the fused systems resulting from the condensation with a phenyl group.

The term “heteroaryl” or aromatic heterocycles refers to a 3 to 14, preferably 5 to 10 membered aromatic hetero, mono-, bi-, or multi-cyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1,2,4-thiadiazolyl, isothiazolyl, triazolyl, tetrazolyl, isoquinolyl, benzothienyl, isobenzofuryl, pyrazolyl, carbazolyl, benzimidazolyl, isoxazolyl, pyridyl-N-oxide, as well as the fused systems resulting from the condensation with a phenyl group.

“Alkyl”, “cycloalkyl”, “alkenyl”, “alkynyl”, “aryl”, “heteroaryl”, “heterocyclic” and the like refer also to the corresponding “alkylene”, “cycloalkylene”, “alkenylene”, “alkynylene”, “arylene”, “heteroarylene”, “heterocyclene” and the likes which are formed by the removal of two hydrogen atoms.

“Arylalkyl” refers to an acyclic alkyl radical in which one of the hydrogen atoms bonded to a carbon atom, typically a terminal or sp³ carbon atom, is replaced with an aryl radical.

Typical arylalkyl groups include, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

“Heteroarylalkyl” refers to an acyclic alkyl radical in which one of the hydrogen atoms bonded to a carbon atom, typically a terminal or sp³ carbon atom, is replaced with a heteroaryl radical. Examples of heteroarylalkyl groups are 2-benzimidazolylmethyl, 2-furylethyl.

Examples of a “hydroxyl protecting group” includes, methoxymethyl ether, 2-methoxyethoxymethyl ether, tetrahydropyranyl ether, benzyl ether, p-methoxybenzyl ether, trimethylsilyl ether, triethylsilyl ether, triisopropylsilyl ether, t-butyldimethylsilyl ether, triphenylmethylsilyl ether, acetate ester, substituted acetate esters, pivaloate, benzoate, methanesulfonate and p-toluenesulfonate.

“Leaving group” refers to a functional group that can be substituted by another functional group. Such leaving groups are well known in the art, and examples include, a halide (e.g., chloride, bromide, and iodide), methanesulfonyl (mesyl), p-toluenesulfonyl (tosyl), trifluoromethylsulfonyl (triflate), and trifluoromethylsulfonate. A preferred leaving group is selected from nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

The following abbreviations may be used herein and have the indicated definitions: Boc, tert-butoxy carbonyl; BroP, bromotrispyrrolidinophosphonium hexafluorophosphate; CDI, 1,1′-carbonyldiimidazole; DCC, dicyclohexylcarbodiimide; DCE, dichloroethane; DCM, dichloromethane; DEAD is diethylazodicarboxylate, DIAD, diisopropylazodicarboxylate; DIBAL-H, diisobutyl-aluminium hydride; DIPEA or DEA, diisopropylethylamine; DEPC, diethyl phosphorocyanidate; DMA, N,N-dimethyl acetamide; DMAP, 4-(N, N-dimethylamino)pyridine; DMF, N,N-dimethylformamide; DMSO, dimethylsulfoxide; DTPA is diethylenetriaminepentaacetic acid; DTT, dithiothreitol; EDC, 1-(3-dimethylaminopropyl)-3-ethylcarbodiimide hydrochloride; ESI-MS, electrospray mass spectrometry; EtOAc is ethyl acetate; Fmoc is N-(9-fluorenylmethoxycarbonyl); HATU, 0-(7-azabenzotriazol-1-yl)-N, N, N′, N′-tetramethyluronium hexafluorophosphate; HOBt, 1-hydroxybenzotriazole; HPLC, high pressure liquid chromatography; NHS, N-Hydroxysuccinimide; MeCN is acetonitrile; MeOH is methanol; MMP, 4-methylmorpholine; PAB, p-aminobenzyl; PBS, phosphate-buffered saline (pH 7.0˜7.5); Ph is phenyl; phe is L-phenylalanine; PyBrop is bromo-tris-pyrrolidino-phosphonium hexafluorophosphate; PEG, polyethylene glycol; SEC, size-exclusion chromatography; TCEP, tris(2-carboxyethyl)phosphine; TFA, trifluoroacetic acid; THF, tetrahydrofuran; Val, valine; TLC is thin layer chromatography; UV is ultraviolet.

The “amino acid(s)” can be natural and/or unnatural amino acids, preferably alpha-amino acids. Natural amino acids are those encoded by the genetic code, which are alanine, arginine, asparagine, aspartic acid, cysteine, glutamic acid, glutamine, glycine, histidine, isoleucine, leucine, lysine, methionine, phenylalanine, proline, serine, threonine, tyrosine. tryptophan and valine. The unnatural amino acids are derived forms of proteinogenic amino acids. Examples include hydroxyproline, lanthionine, 2-aminoisobutyric acid, dehydroalanine, gamma-aminobutyric acid (the neurotransmitter), ornithine, citrulline, beta alanine (3-aminopropanoic acid), gamma-carboxyglutamate, selenocysteine (present in many noneukaryotes as well as most eukaryotes, but not coded directly by DNA), pyrrolysine (found only in some archaea and one bacterium), N-formylmethionine (which is often the initial amino acid of proteins in bacteria, mitochondria, and chloroplasts), 5-hydroxytryptophan, L-dihydroxyphenylalanine, triiodothyronine, L-3,4-dihydroxyphenylalanine (DOPA), and O-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, 3-aminopropanoic acid, 4-aminobutanoic acid, 5-aminopentanoic acid, 6-aminohexanoic acid, 7-aminoheptanoic acid, methionine-sulfoxide, and methionine methyl sulfonium. Preferably, an amino acid mimetic is a compound that has a structure different from the general chemical structure of an alpha-amino acid but functions in a manner similar to one. The term “unnatural amino acid” is intended to represent the “D” stereochemical form, the natural amino acids being of the “L” form. When 1-8 amino acids are used in this patent application, amino acid sequence is then preferably a cleavage recognition sequence for a protease. Many cleavage recognition sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); and Bouvier et al. Meth. Enzymol. 248: 614 (1995); the disclosures of which are incorporated herein by reference. In particular, the sequence is selected from the group consisting of Val-Cit, Ala-Val, Ala-Ala, Val-Val, Val-Ala-Val, Lys-Lys, Ala-Asn-Val, Val-Leu-Lys, Cit-Cit, Val-Lys, Ala-Ala-Asn, Asp-Lys, Asp-Glu, Glu-Lys, Lys, Cit, Ser, and Glu.

The “glycoside” is a molecule in which a sugar group is bonded through its anomeric carbon to another group via a glycosidic bond. Glycosides can be linked by an O— (an O-glycoside), N— (a glycosylamine), S— (a thioglycoside), or C— (a C-glycoside) glycosidic bond. Its core the empirical formula is Cₘ(H₂O)ₙ (where m could be different from n, and m and n are <36), Glycoside herein includes glucose (dextrose), fructose (levulose) allose, altrose, mannose, gulose, iodose, galactose, talose, galactosamine, glucosamine, sialic acid, N-acetylglucosamine, sulfoquinovose (6-deoxy-6-sulfo-D-glucopyranose), ribose, arabinose, xylose, lyxose, sorbitol, mannitol, sucrose, lactose, maltose, trehalose, maltodextrins, raffinose, Glucuronic acid (glucuronide), and stachyose. It can be in D form or L form, 5 atoms cyclic furanose forms, 6 atoms cyclic pyranose forms, or acyclic form, α-isomer (the —OH of the anomeric carbon below the plane of the carbon atoms of Haworth projection), or a β-isomer (the —OH of the anomeric carbon above the plane of Haworth projection). It is used herein as a monosaccharide, disaccharide, polyols, or oligosaccharides containing 3-6 sugar units.

The term “antibody,” as used herein, refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce auto-immune antibodies associated with an autoimmune disease. The immunoglobulin disclosed herein can be of any type (e.g. IgG, IgE, IgM, IgD, IgA and IgY), class (e.g., IgG1, IgG2, IgG3, IgG4, IgA1 and IgA2) or subclass of immunoglobulin molecule. The immunoglobulins can be derived from any species. Preferably, however, the immunoglobulin is of human, murine, or rabbit origin. Antibodies useful in the invention are preferably monoclonal, and include, but are not limited to, polyclonal, monoclonal, bispecific, human, humanized or chimeric antibodies, single chain antibodies, Fv, Fab fragments, F(ab′) fragments, F(ab′)₂ fragments, fragments produced by a Fab expression library, anti-idiotypic (anti-Id) antibodies, CDR's, and epitope-binding fragments of any of the above which immunospecifically bind to cancer cell antigens, viral antigens or microbial antigens.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

An “intact antibody” is one which comprises an antigen-binding variable region as well as a light chain constant domain (C_{L}) and heavy chain constant domains, C_{H1}, C_{H2}, C_{H3} and C_{H4}, as appropriate for the antibody class. The constant domains may be native sequence constant domains (e.g., human native sequence constant domains) or amino acid sequence variant thereof.

An “antibody fragment” comprises a portion of an intact antibody, comprising the antigen-binding or variable region thereof. Examples of antibody fragments include Fab, Fab′, F(ab′).sub.2, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is an entity to which an antibody specifically binds.

The terms “specific binding” and “specifically binds” mean that the antibody or antibody derivative will bind, in a highly selective manner, with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

An “enantiomer”, also known as an “optical isomer”, is one of two stereoisomers that are mirror images of each other that are non-superposable (not identical), much as one's left and right hands are the same except for being reversed along one axis (the hands cannot be made to appear identical simply by reorientation). A single chiral atom or similar structural feature in a compound causes that compound to have two possible structures which are non-superposable, each a mirror image of the other. The presence of multiple chiral features in a given compound increases the number of geometric forms possible, though there may be some perfect-mirror-image pairs. Enantiopure compounds refer to samples having, within the limits of detection, molecules of only one chirality. When present in a symmetric environment, enantiomers have identical chemical and physical properties except for their ability to rotate plane-polarized light (+/−) by equal amounts but in opposite directions (although the polarized light can be considered an asymmetric medium). They are sometimes called optical isomers for this reason. A mixture of equal parts of an optically active isomer and its enantiomer is termed racemic and has zero net rotation of plane-polarized light because the positive rotation of each (+) form is exactly counteracted by the negative rotation of a (−) one. Enantiomer members often have different chemical reactions with other enantiomer substances. Since many biological molecules are enantiomers, there is sometimes a marked difference in the effects of two enantiomers on biological organisms. In drugs, for example, often only one of a drug's enantiomers is responsible for the desired physiologic effects, while the other enantiomer is less active, inactive, or sometimes even productive of adverse effects. Owing to this discovery, drugs composed of only one enantiomer (“enantiopure”) can be developed to enhance the pharmacological efficacy and sometimes eliminate some side effects.

Isotopes are variants of a particular chemical element which differs in neutron number. All isotopes of a given element have the same number of protons in each atom. Each atomic number identifies a specific element, but not the isotope; an atom of a given element may have a wide range in its number of neutrons. The number of nucleons (both protons and neutrons) in the nucleus is the atom's mass number, and each isotope of a given element has a different mass number. For example, carbon-12, carbon-13 and carbon-14 are three isotopes of the element carbon with mass numbers 12, 13 and 14 respectively. The atomic number of carbons is 6, which means that every carbon atom has 6 protons, so that the neutron numbers of these isotopes are 6, 7 and 8 respectively. Hydrogen atom has three isotopes of protium (¹H), deuterium (²H), and tritium (³H), which deuterium has twice the mass of protium and tritium has three times the mass of protium. Isotopic substitution can be used to determine the mechanism of a chemical reaction and via the kinetic isotope effect. Isotopic substitution can be used to study how the body affects a specific xenobiotic/chemical after administration through the mechanisms of absorption and distribution, as well as the metabolic changes of the substance in the body (e.g. by metabolic enzymes such as cytochrome P450 or glucuronosyltransferase enzymes), and the effects and routes of excretion of the metabolites of the drug. This study is called pharmacokinetics (PK). Isotopic substitution can be used to study of the biochemical and physiologic effects of drugs. The effects can include those manifested within animals (including humans), microorganisms, or combinations of organisms (for example, infection). This study is called pharmacodynamics (PD). The effects can include those manifested within animals (including humans), microorganisms, or combinations of organisms (for example, infection). Both together influence dosing, benefit, and adverse effects of the drug. isotopes can contain a stable (non-radioactive) or an unstable element. Isotopic substitution of a drug may have a different therapeutical efficacy of the original drug. By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art. Benefits may also be obtained from replacement of normally abundant ¹²C with ¹³C.

“Pharmaceutically” or “pharmaceutically acceptable” refer to molecular entities and compositions that do not produce an adverse, allergic or other untoward reaction when administered to an animal, or a human, as appropriate.

“Pharmaceutically acceptable solvate” or “solvate” refer to an association of one or more solvent molecules and a disclosed compound. Examples of solvents that form pharmaceutically acceptable solvates include, but are not limited to, water, isopropanol, ethanol, methanol, DMSO, ethyl acetate, acetic acid and ethanolamine.

“Pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions as suitable therapeutic combinations.

As used herein, “pharmaceutical salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucuronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutical salts of the present invention can be synthesized from the parent compound which contains a basic or acidic moiety by conventional chemical methods. Generally, such salts can be prepared via reaction the free acidic or basic forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, the disclosure of which is hereby incorporated by reference.

The term “therapeutically effective amount” refers to an amount of a conjugate effective to treat a disease or disorder in a mammal. In the case of cancer, the therapeutically effective amount of the conjugate may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

The term “pharmaceutically acceptable form” as used herein refers to a form of a disclosed compound including, but is not limited to, pharmaceutically acceptable salts, esters, hydrates, solvates, polymorphs, isomers, prodrugs, and isotopically labeled derivatives thereof. In one embodiment, a “pharmaceutically acceptable form” includes, but is not limited to, pharmaceutically acceptable salts, esters, prodrugs and isotopically labeled derivatives thereof.

In some embodiments, a “pharmaceutically acceptable form” includes, but is not limited to, pharmaceutically acceptable isomers and stereoisomers, prodrugs and isotopically labeled derivatives thereof.

The term “substantial” or “substantially” refers to a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

The term “cytotoxic activity” refers to a cell-killing effect of a drug or Camptothecin Conjugate or an intracellular metabolite of a Camptothecin Conjugate. Cytotoxic activity may be expressed as the IC.sub.50 value, which is the concentration (molar or mass) per unit volume at which half the cells survive.

The term “cytostatic activity” refers to an anti-proliferative effect of a drug or Camptothecin analog Conjugate or an intracellular metabolite of a Camptothecin Conjugate.

The term “cytotoxic agent” as used herein refers to a substance that has cytotoxic activity and causes destruction of cells. The term is intended to include chemotherapeutic agents, and toxins such as small molecule toxins or enzymatically active toxins of bacterial, fungal, plant or animal origin, including synthetic analogs and derivatives thereof.

“Administering” or “administration” refers to any mode of transferring, delivering, introducing or transporting a pharmaceutical drug or other agent to a subject. Such modes include oral administration, topical contact, intravenous, intraperitoneal, intramuscular, intralesional, intranasal, subcutaneous or intrathecal administration. Also contemplated by the present invention is utilization of a device or instrument in administering an agent. Such device may utilize active or passive transport and may be slow-release or fast-release delivery device.

In the context of cancer, the term “treating” includes any or all of: preventing growth of tumor cells or cancer cells, preventing replication of tumor cells or cancer cells, lessening of overall tumor burden and ameliorating one or more symptoms associated with the disease.

In the context of an autoimmune disease, the term “treating” includes any or all of: preventing replication of cells associated with an autoimmune disease state including, but not limited to, cells capable of producing an autoimmune antibody, lessening the autoimmune-antibody burden and ameliorating one or more symptoms of an autoimmune disease.

In the context of an infectious disease, the term “treating” includes any or all of: preventing the growth, multiplication or replication of the pathogen that causes the infectious disease and ameliorating one or more symptoms of an infectious disease.

The terms “cancer” and “cancerous” refer to or describe the physiological condition or disorder in mammals that is typically characterized by unregulated cell growth. A “tumor” comprises one or more cancerous cells.

An “autoimmune disease” as used herein refers to a disease or disorder arising from and directed against an individual's own tissues or proteins.

“Patient” as used herein refers to a subject to whom is administered a Camptothecin Conjugate of the present invention. Patient includes, but are not limited to, a human, rat, mouse, guinea pig, non-human primate, pig, goat, cow, horse, dog, cat, bird and fowl. Typically, the patient is a rat, mouse, dog, human or non-human primate, more typically a human.

Examples of a “mammal” or “animal” include, but are not limited to, a human, rat, mouse, guinea pig, monkey, pig, goat, cow, horse, dog, cat, bird and fowl.

The terms “treat” or “treatment,” unless otherwise indicated by context, refer to therapeutic treatment and prophylactic wherein the object is to inhibit or slow down (lessen) an undesired physiological change or disorder, such as the development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

### Drug-Linker-Binding Ligand Conjugates

As stated above, this invention provides a cell surface-binding molecule-camptothecin analog conjugate of Formula (I):

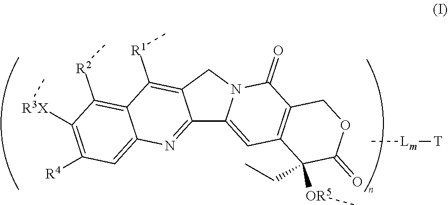

or their pharmaceutically acceptable salts, hydrates, or hydrated salts; or the polymorphic crystalline structures of these compounds; or their isotopes, optical isomers, racemates, diastereomers or enantiomers thereof;

Wherein T is a targeting or binding ligand; L is a releasable linker; ----- is a linkage bond that L connects to an atom of R¹, R², R³ or R⁵ independently inside the bracket independently; n is 1-30 and m is 1-10;

Inside the bracket is a potent amptothecin analog wherein:

R¹ and R² are independently H; linear or branched C₁-C₆ of alkyl, alkyl alcohol, alkyl amine (including primary, secondary, tertiary amine, or quaternary ammonium), aminoalkyl, oxylalkyl, aminoalkylamino, oxylalkylamino, aminoalkyloxyl, oxylalkyloxyl, alkyl carboxylic acid, or carbonyl; C₂-C₆ of heteroalkyl, alkylcycloalkyl, heterocycloalkyl, heterocyclic, aminocycloalkyl, heteroalkylcycloalkyl, alkylcarbonyl, aminoalkylcarbonyl, oxylalkylcarbonyl, alkyl ether, alkyl ester, alkyl amide, oxylalkylamide, aminoalkylamide, oxime; NH₂, or OH;

R³ is independently H, C(O)NH, C(O)O, SO₂R⁶, SO₃R⁶, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR^{6′})₂, C(O)R⁶, C(O)NHR⁶; linear or branched C₁-C₆ of alkyl, alkyl alcohol, alkyl amine (including primary, secondary, tertiary amine, or quaternary ammonium), or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide, oxime; C₅˜C₁₂ glycoside, NH₂, or OH;

R⁴ is halo (F, Cl, Br, or I), CN, NO₂, SO₃H, OR⁶, SR⁶, S(O₂)R⁶, NHR⁶, N(R⁶)(R^{6′}), C(O)XR⁶, N+(R⁶)(R^{6′})(R^{6″)};

X is NH or O;

R⁵ is H, C(O)O, C(O)NH, R⁶C(O), linear or branched C₁-C₆ of alkyl, alkyl alcohol, alkyl amine (including primary, secondary, tertiary amine, or quaternary ammonium), alkyl carboxylic acid; C₂-C₆ of carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;

R⁶, R^{6′}, and R^{6″} are independently H, C₁-C₆ of alkyl, alkyl alcohol, alkyl amine (including primary, secondary, tertiary amine, or quaternary ammonium) or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or pharmaceutical salts; In addition, R¹, R², R³ and R⁶ can be independently absent, and R², R³, X, C-10 and C-9 can together form a 5-, 6- or 7-member heterocyclic ring.

In one specific embodiment, conjugates of camptothecin analogs have the formula (II)

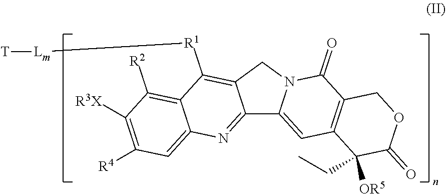

or their pharmaceutically acceptable salts, hydrates, or hydrated salts; or the polymorphic crystalline structures of these compounds; or their isotopes, optical isomers, racemates, diastereomers or enantiomers thereof;

Wherein T is a targeting or binding ligand; L is a releasable linker; n is 1-30 and m is 1-10;

Inside the bracket is a potent camptothecin analog wherein:

R¹ is linear or branched C₁-C₆ of alkyl, alkyloxyl, alkyl amino (including primary, secondary, tertiary amino, or quaternary ammonium), oxylcarbonyl, aminocarbonyl, aminoalkyl, oxylalkyl, aminoalkylamino, oxylalkylamino, aminoalkyloxyl, oxylalkyloxyl, or alkyl carboxylic; C₂-C₆ of heteroalkyl, alkylcycloalkyl, heterocycloalkyl, heterocyclic, oxylcycloalkyl, aminocycloalkyl, heteroalkylcycloalkyl, alkylcarbonyl, aminoalkylcarbonyl, oxylalkylcarbonyl, alkyl ether, alkyl ester, alkyl amide, oxylalkylamide, aminoalkylamide, oxime; NH, or O;

R² is H, linear or branched C₁-C₆ of alkyl, alkyl alcohol, alkyl amine (including primary, secondary, tertiary amine, or quaternary ammonium), aminoalkyl alcohol, aminoalkyl amine, oxylalkyl alcohol, oxylalkyl amine, aminoalkyl, oxylalkyl, or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide, oxime; NH₂, or OH;

R³ is independently H, R⁶NHC(O), R⁶OC(O), SO₂R⁶, SO₃R⁶, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR^{6′})₂, R⁶C(O), C(O)NR⁶R^{6′}; linear or branched C₁-C₆ of alkyl, alkyl alcohol, alkyl amine (including primary, secondary, tertiary amine, or quaternary ammonium), or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide, oxime; C₅˜C₁₂ glycoside;

R⁴ is halo (F, Cl, Br, or I), CN, NO₂, SO₃H, OR⁶, SR⁶, S(O₂)R⁶, NHR⁶, N(R⁶)(R^{6′}), C(O)XR⁶, N+(R⁶)(R^{6′})(R^{6″});

X is NH or O;

R⁵ is H, C(O)OR⁶, C(O)NHR⁶, R⁶C(O), linear or branched C₁-C₆ of alkyl, alkyl alcohol, alkyl amine (including primary, secondary, tertiary amine, or quaternary ammonium), alkyl carboxylic acid; C₂-C₆ of carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;

R⁶, R^{6′}, and R^{6″} are independently H, C₁-C₆ of alkyl, alkyl alcohol, alkyl amine (including primary, secondary, tertiary amine, or quaternary ammonium) or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or pharmaceutical salts;

In addition, R¹ can be absent and C-7 directly links to L, and R², R³, X, C-10 and C-9 can join together to form a 5-, 6- or 7-member heterocyclic ring.

Illustrative compounds inside the bracket of formula (II) have the structures:

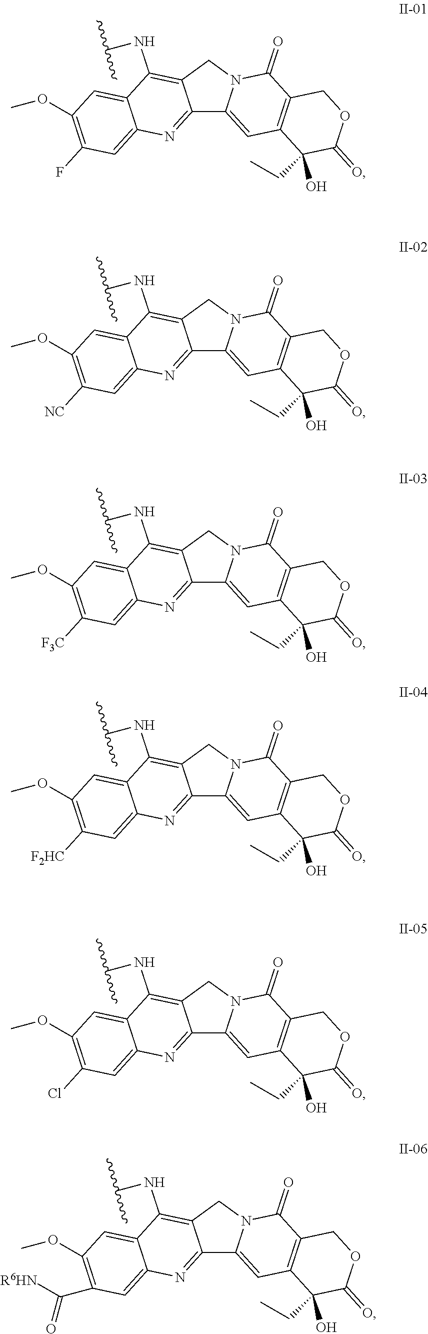

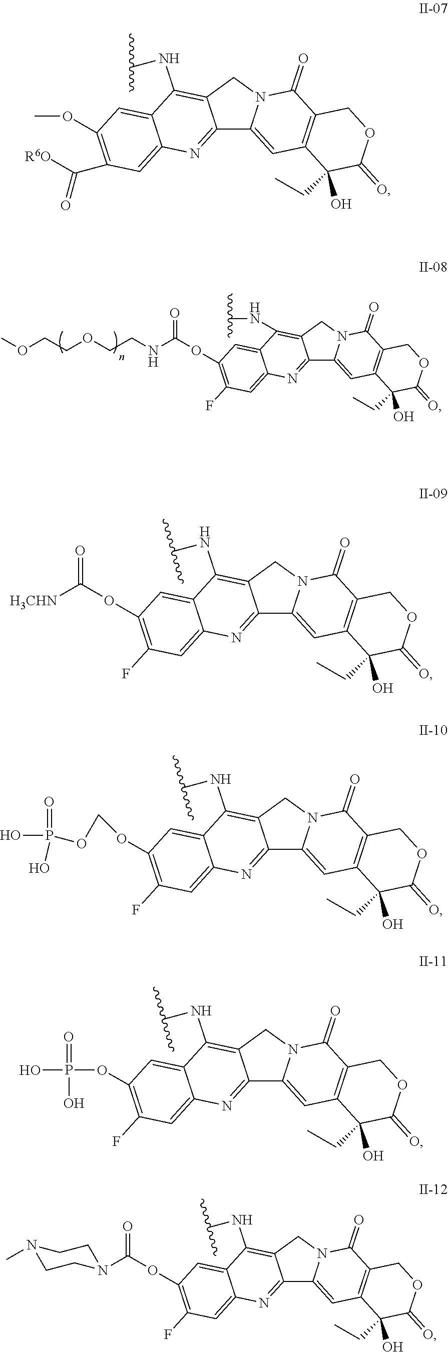

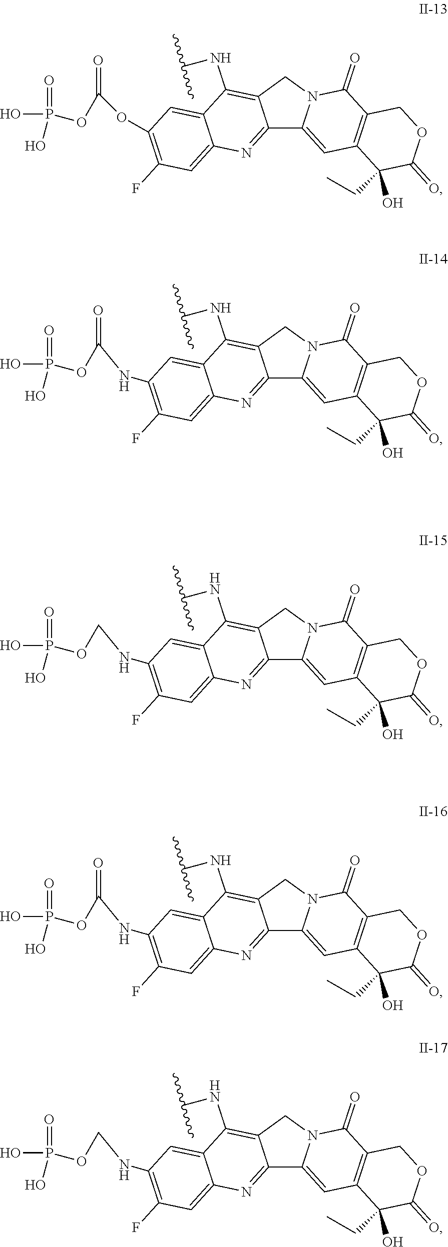 [SMILES: *Nc1c2c(nc3cc(F)c(OC(=O)OP(=O)(O)O)cc13)-c1cc3c(c(=O)n1C2)COC(=O)[C@]3(O)CC ; *Nc1c2c(nc3cc(F)c(NC(=O)OP(=O)(O)O)cc13)-c1cc3c(c(=O)n1C2)COC(=O)[C@]3(O)CC ; *Nc1c2c(nc3cc(F)c(NCOP(=O)(O)O)cc13)-c1cc3c(c(=O)n1C2)COC(=O)[C@]3(O)CC ; *Nc1c2c(nc3cc(F)c(NC(=O)OP(=O)(O)O)cc13)-c1cc3c(c(=O)n1C2)COC(=O)[C@]3(O)CC ; *Nc1c2c(nc3cc(F)c(NCOP(=O)(O)O)cc13)-c1cc3c(c(=O)n1C2)COC(=O)[C@]3(O)CC]

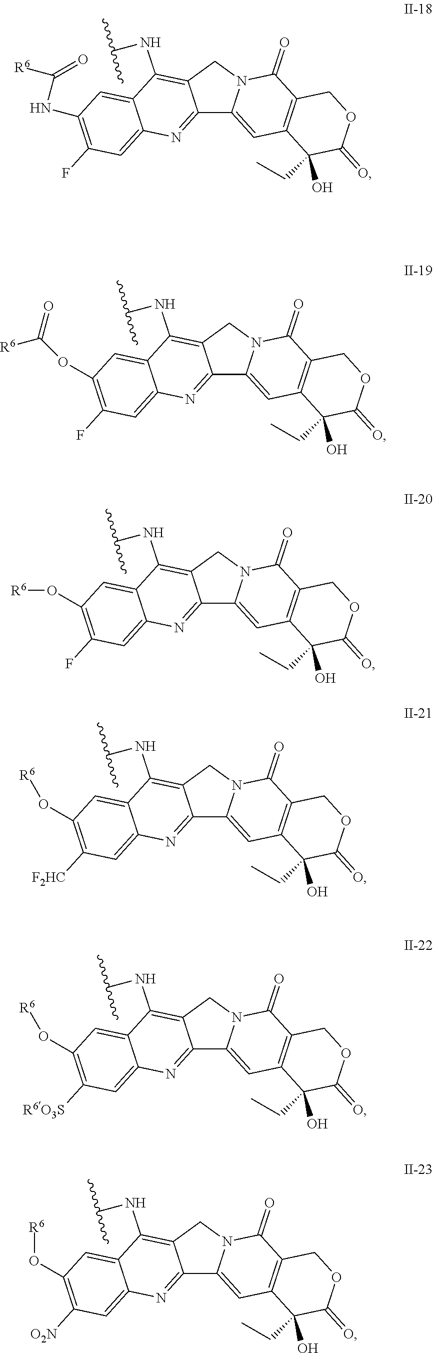

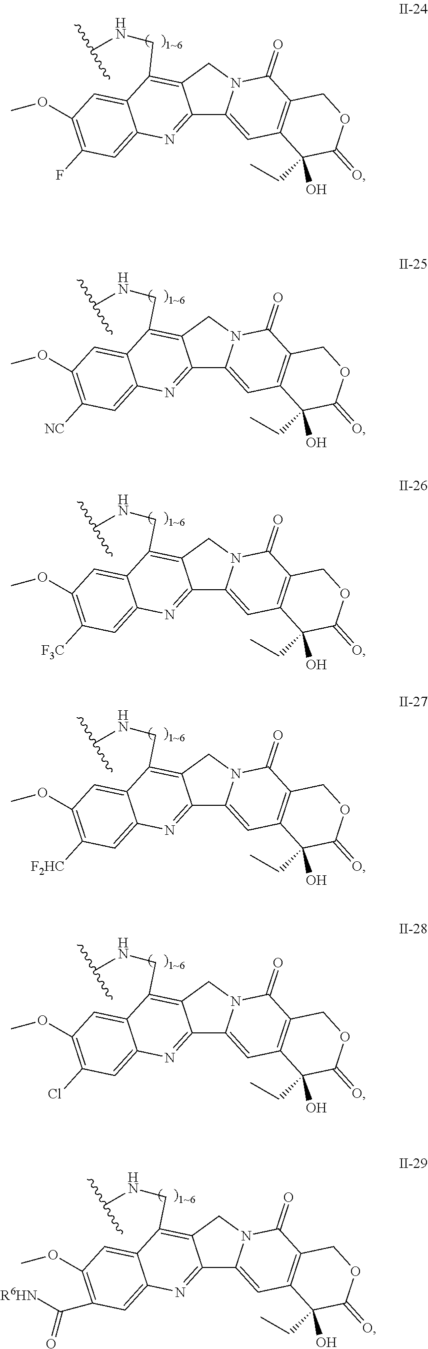

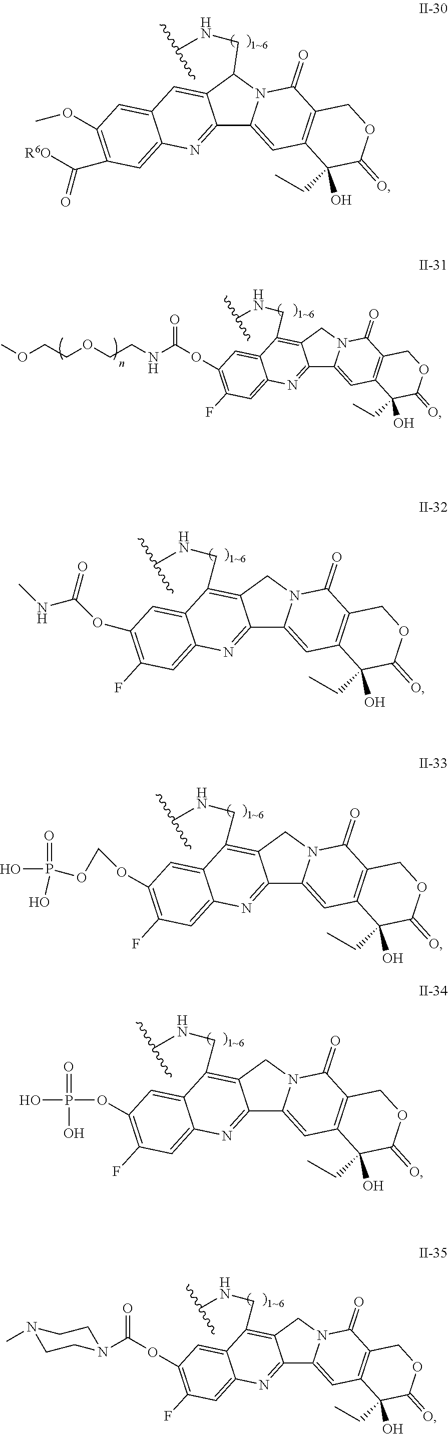

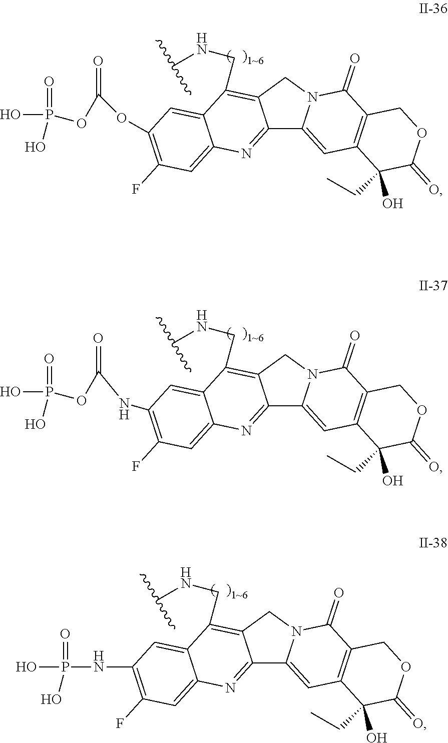

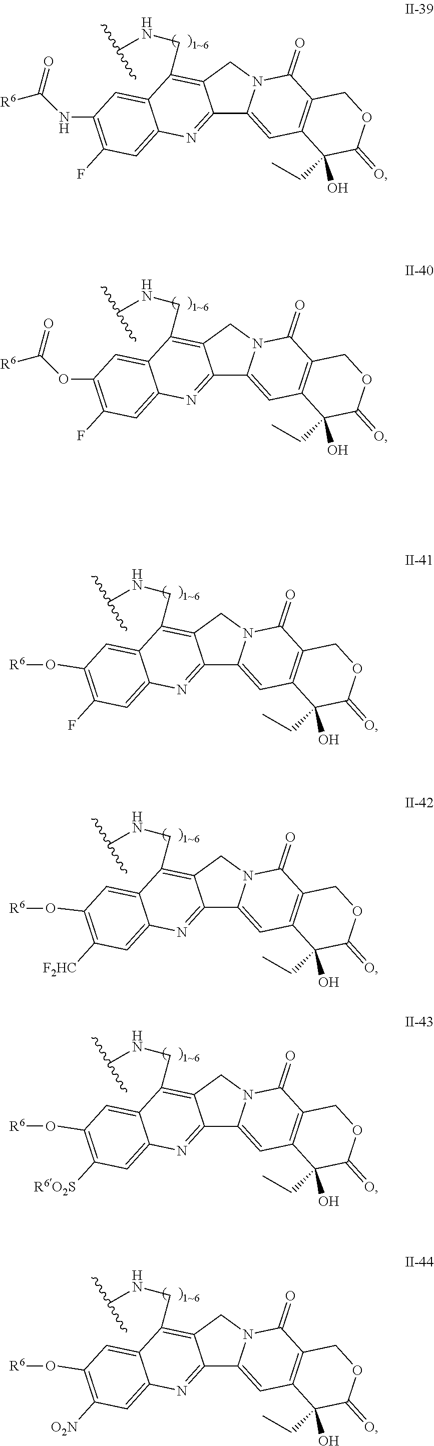

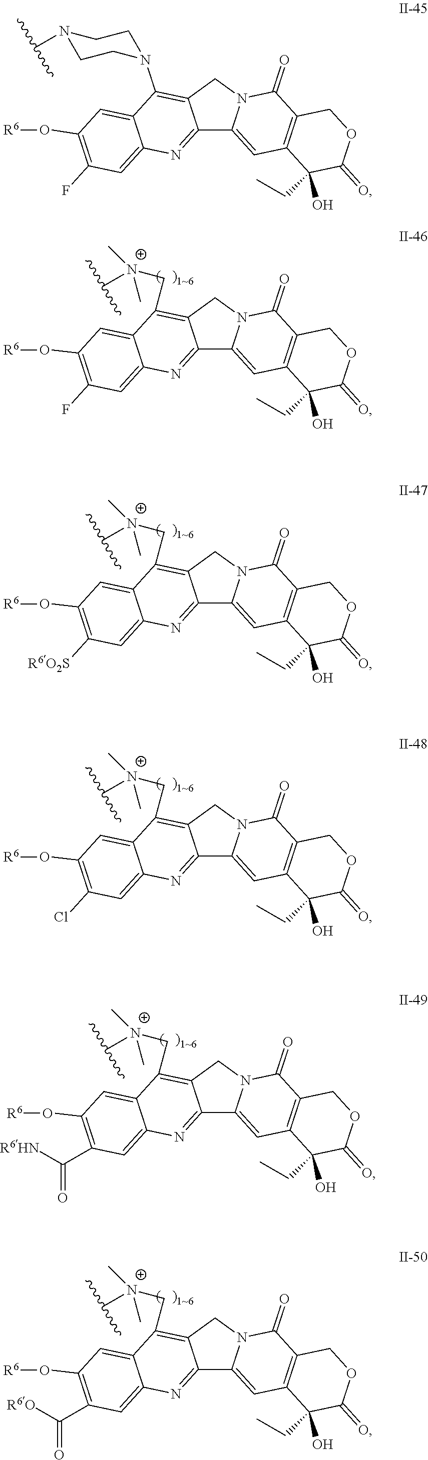

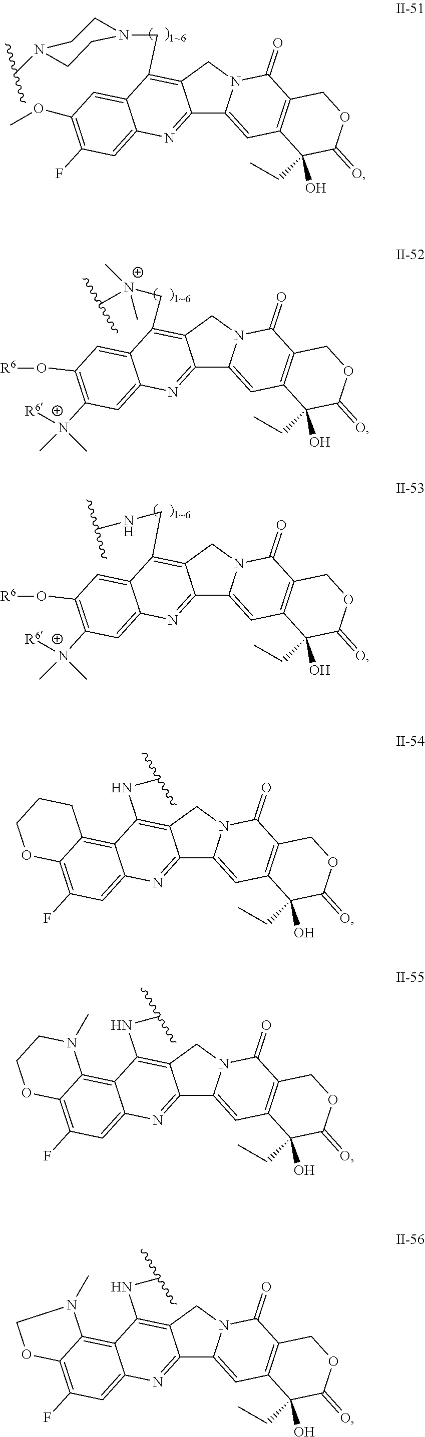

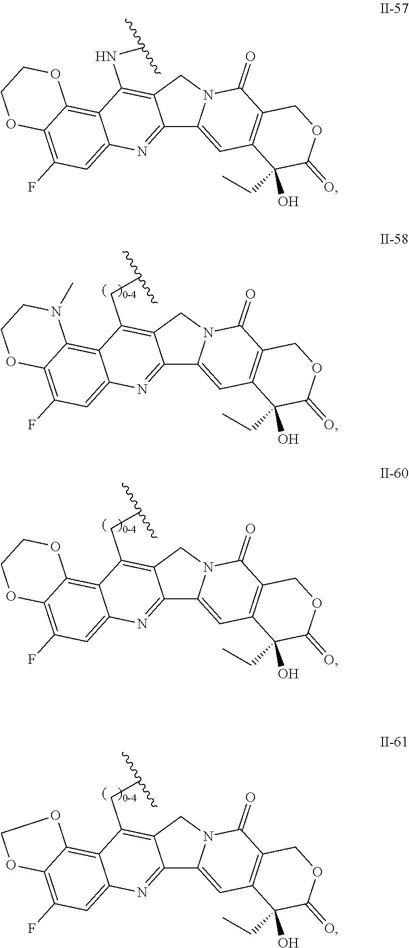

or their pharmaceutically acceptable salts, hydrates, or hydrated salts; or the polymorphic crystalline structures of these compounds; or their isotope, optical isomers, racemates, diastereomers or enantiomers;

wherein R⁶ and R^{6′} are defined the same above.

wherein [symbol] is the site that linked to a linker L of Formula (II).

In another specific embodiment, a conjugate of a cell-binding molecule-camptothecin analog has the Formula (III):

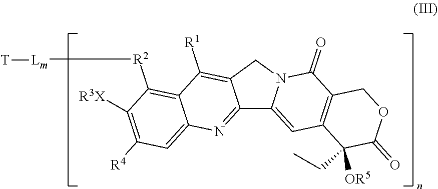

or their pharmaceutically acceptable salts, hydrates, or hydrated salts; or the polymorphic crystalline structures of these compounds; or their isotopes, optical isomers, racemates, diastereomers or enantiomers thereof;

Wherein T is a targeting or binding ligand; L is a releasable linker; n is 1-30 and m is 1-10;

Inside the bracket is a potent camptothecin analog wherein:

R¹ is linear or branched C₁-C₆ of alkyl, alkyloxyl, alkyl amino (including primary, secondary, tertiary amino, or quaternary ammonium), oxylcarbonyl, aminocarbonyl, aminoalkyl, oxylalkyl, aminoalkylamino, oxylalkylamino, aminoalkyloxyl, oxylalkyloxyl, or alkyl carboxylic; C₂-C₆ of heteroalkyl, alkylcycloalkyl, heterocycloalkyl, heterocyclic, oxylcycloalkyl, aminocycloalkyl, heteroalkylcycloalkyl, alkylcarbonyl, aminoalkylcarbonyl, oxylalkylcarbonyl, alkyl ether, alkyl ester, alkyl amide, oxylalkylether, aminoalkylether, oxylalkylester, aminoalkylester, oxylalkylamide, aminoalkylamide, oxime; NH, or O;

R² is NH, NR⁶, —N⁺R⁶R^{6′}—, O, S, linear or branched C₁-C₆ of alkyl, alkyl alcohol, alkyl amine (including primary, secondary, tertiary amine, or quaternary ammonium), aminoalkyl alcohol, aminoalkyl amine, oxylalkyl alcohol, oxylalkyl amine, aminoalkyl, oxylalkyl, or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide, oxime; oxylalkylether, aminoalkylether, oxylalkylester, aminoalkylester, oxylalkylamide, aminoalkylamide;

R³ is independently H, R⁶NHC(O), R⁶OC(O), SO₂R⁶, SO₃R⁶, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR^{6′})₂, R⁶C(O), C(O)N R⁶R^{6′}; linear or branched C₁-C₆ of alkyl, alkyl alcohol, alkyl amine (including primary, secondary, tertiary amine, or quaternary ammonium), or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide, oxime; C₅˜C₁₂ glycoside;

R⁴ is halo (F, Cl, Br, or I), CN, NO₂, SO₃H, OR⁶, SR⁶, S(O₂)R⁶, NHR⁶, N(R⁶)(R^{6′}), C(O)XR⁶, N+(R⁶)(R^{6′})(R^{6″});

X is NH or O;

R⁵ is H, C(O)OR⁶, C(O)NHR⁶, R⁶C(O), linear or branched C₁-C₆ of alkyl, alkyl alcohol, alkyl amine (including primary, secondary, tertiary amine, or quaternary ammonium), alkyl carboxylic acid; C₂-C₆ of carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;

R⁶, R^{6′}, and R^{6″} are independently H, C₁-C₆ of alkyl, alkyl alcohol, alkyl amine (including primary, secondary, tertiary amine, or quaternary ammonium) or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or pharmaceutical salts;

In addition, R² can be absent and C-9 directly links to L, and R², R³, X, C-10 and C-9 can join together to form a 5-, 6- or 7-member heterocyclic ring.

Illustrative compounds inside the bracket of Formula (III) have the structures:

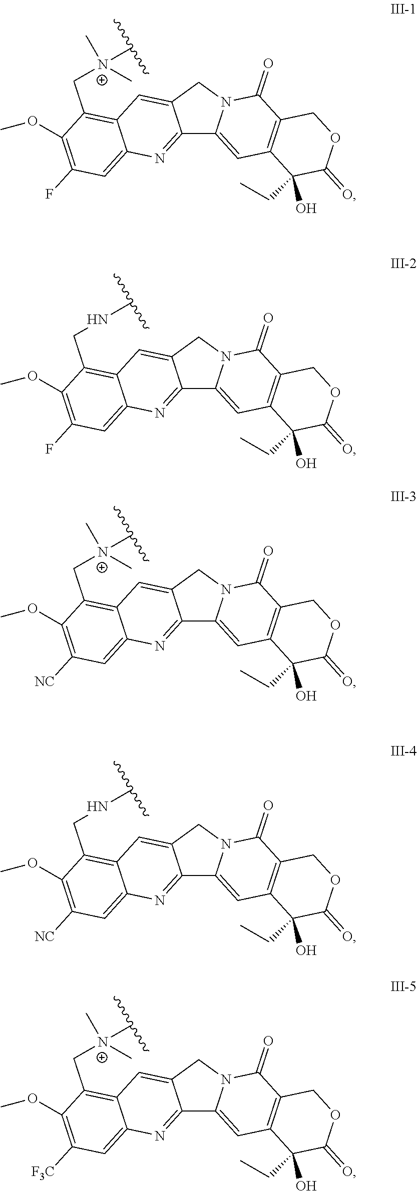 [SMILES: *[N+](C)(C)Cc1c(OC)c(F)cc2nc3c(cc12)Cn1c-3cc2c(c1=O)COC(=O)[C@]2(O)CC ; *NCc1c(OC)c(F)cc2nc3c(cc12)Cn1c-3cc2c(c1=O)COC(=O)[C@]2(O)CC ; *[N+](C)(C)Cc1c(OC)c(C#N)cc2nc3c(cc12)Cn1c-3cc2c(c1=O)COC(=O)[C@]2(O)CC ; *NCc1c(OC)c(C#N)cc2nc3c(cc12)Cn1c-3cc2c(c1=O)COC(=O)[C@]2(O)CC ; *[N+](C)(C)Cc1c(OC)c(C(F)(F)F)cc2nc3c(cc12)Cn1c-3cc2c(c1=O)COC(=O)[C@]2(O)CC]

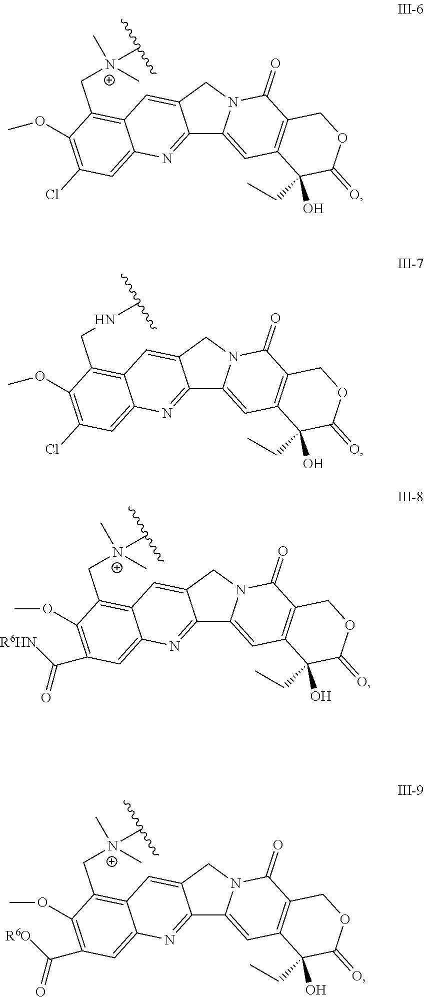

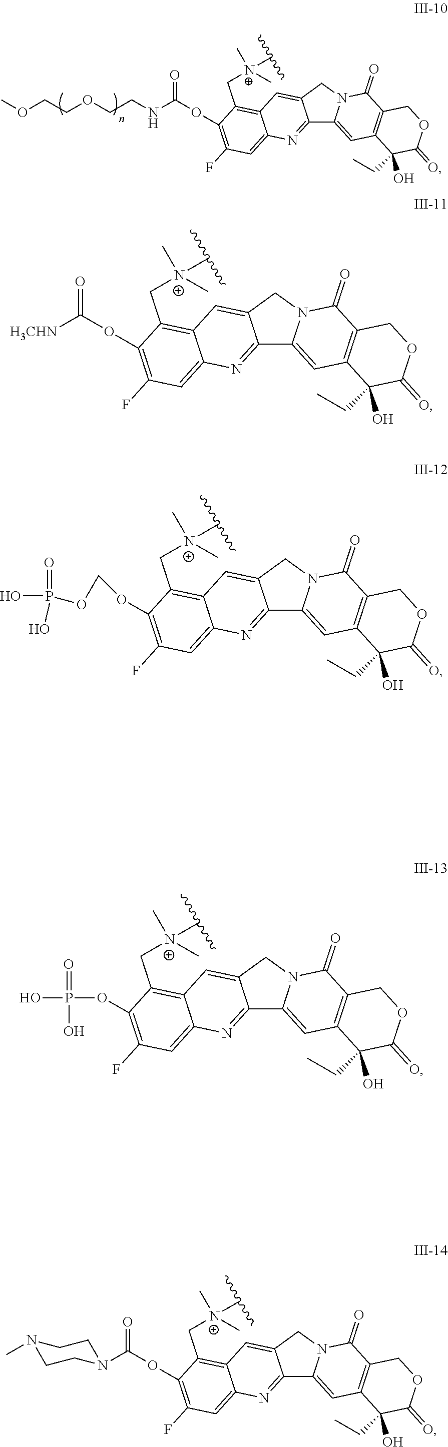

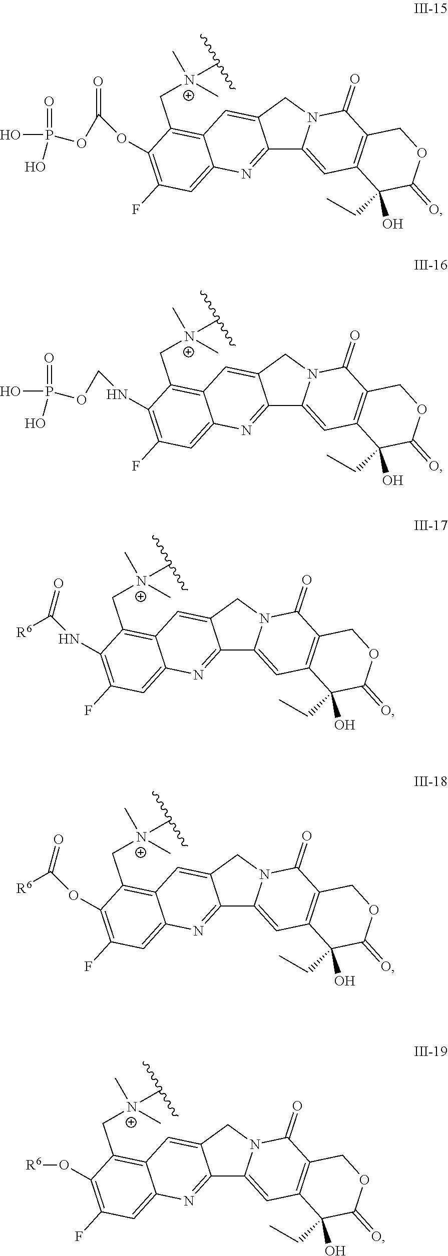

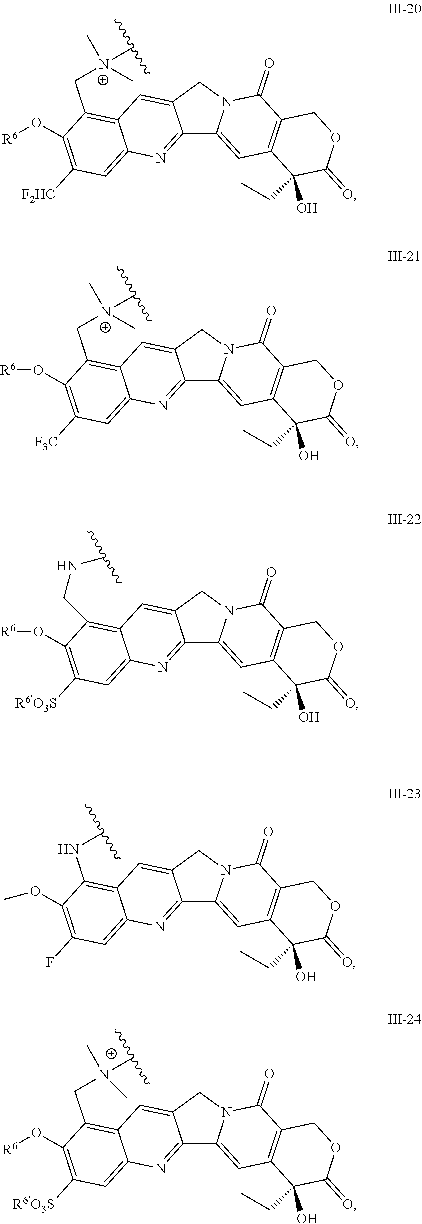

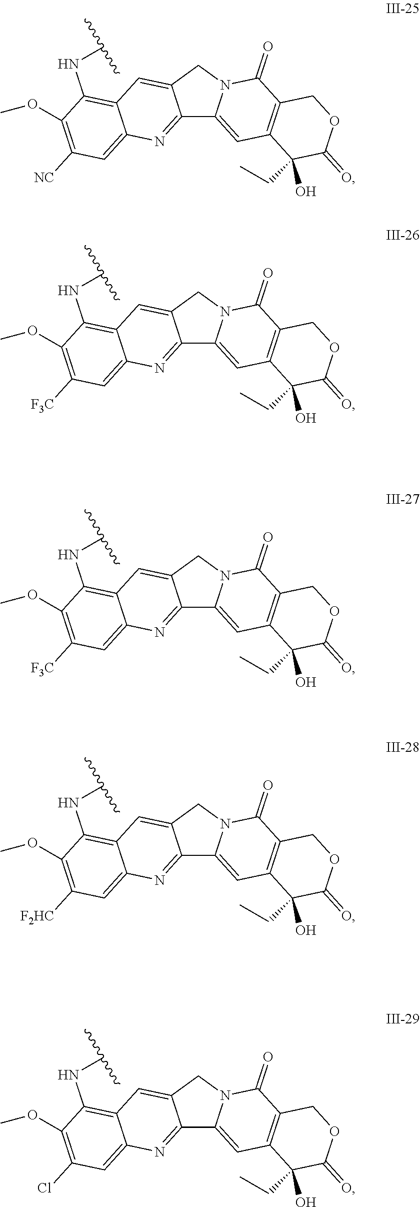 [SMILES: *Nc1c(OC)c(C#N)cc2nc3c(cc12)Cn1c-3cc2c(c1=O)COC(=O)[C@]2(O)CC ; *Nc1c(OC)c(C(F)(F)F)cc2nc3c(cc12)Cn1c-3cc2c(c1=O)COC(=O)[C@]2(O)CC ; *Nc1c(OC)c(C(F)(F)F)cc2nc3c(cc12)Cn1c-3cc2c(c1=O)COC(=O)[C@]2(O)CC ; *Nc1c(OC)c(C(F)F)cc2nc3c(cc12)Cn1c-3cc2c(c1=O)COC(=O)[C@]2(O)CC ; *Nc1c(OC)c(Cl)cc2nc3c(cc12)Cn1c-3cc2c(c1=O)COC(=O)[C@]2(O)CC]

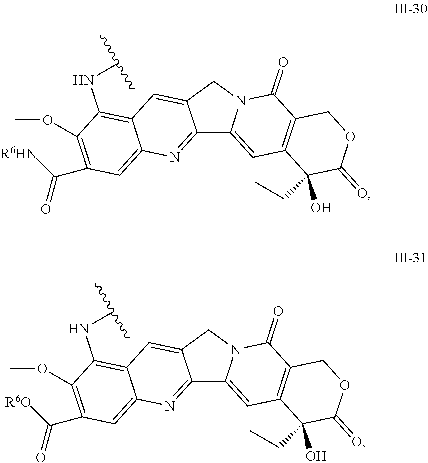

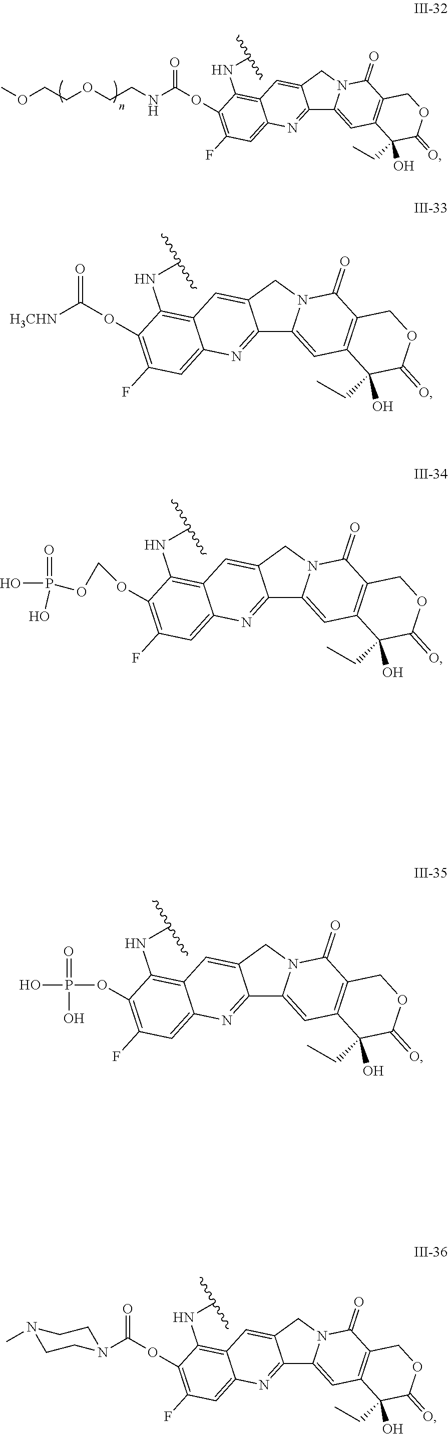

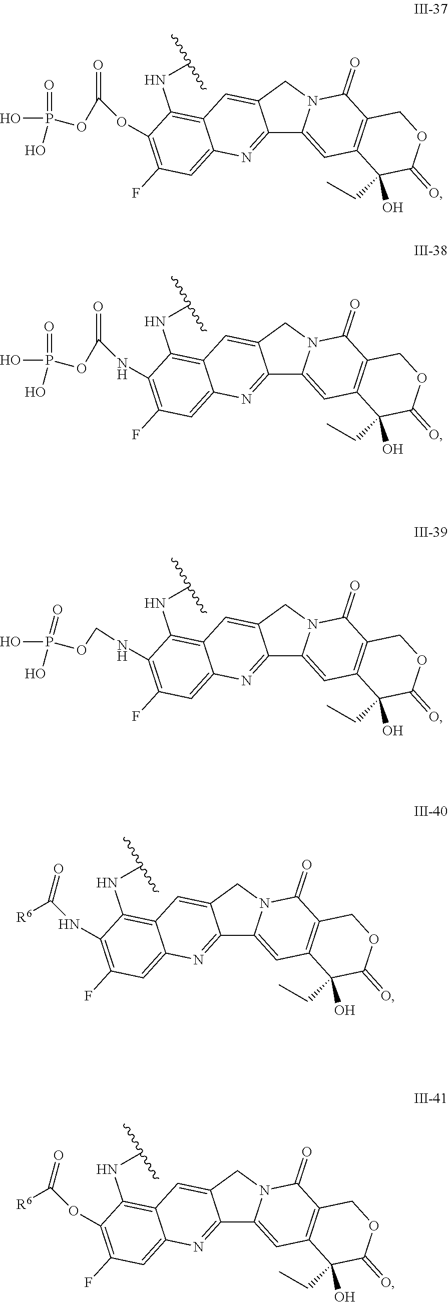

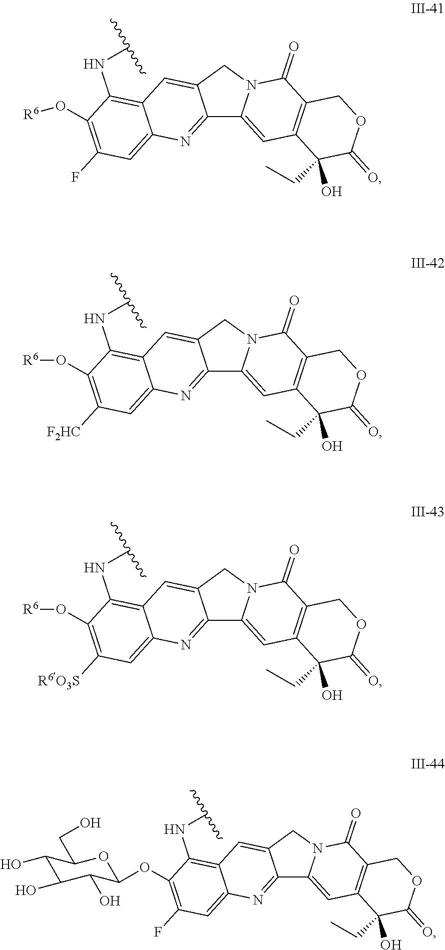

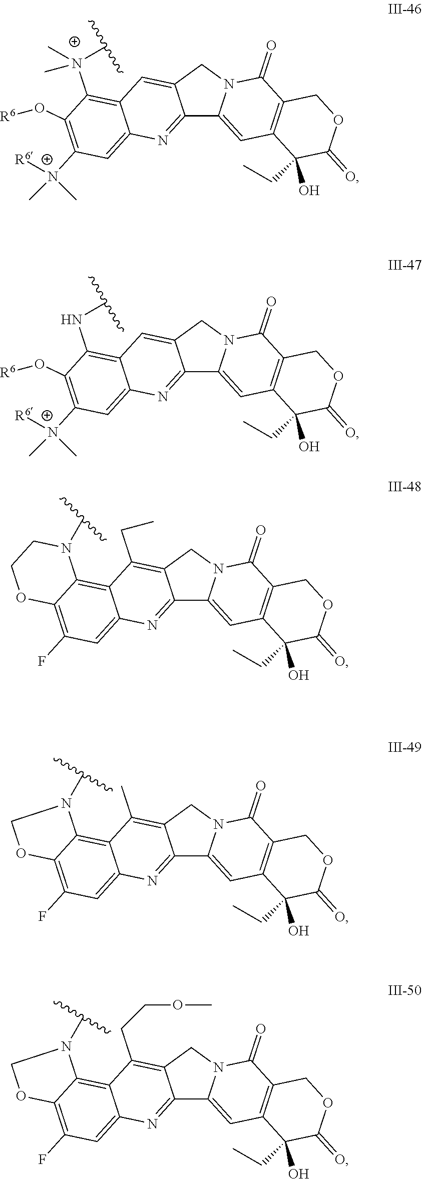

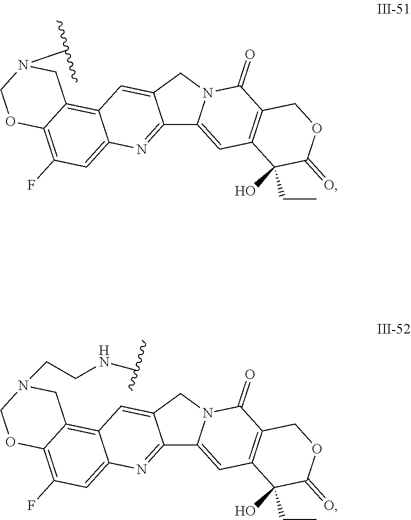 [SMILES: *N1COc2c(F)cc3nc4c(cc3c2C1)Cn1c-4cc2c(c1=O)COC(=O)[C@]2(O)CC ; *NCCN1COc2c(F)cc3nc4c(cc3c2C1)Cn1c-4cc2c(c1=O)COC(=O)[C@]2(O)CC]

or their pharmaceutically acceptable salts, hydrates, or hydrated salts; or the polymorphic crystalline structures of these compounds; or their optical isomers, racemates, diastereomers or enantiomers;

wherein R⁶, and R^{6′} are independently H, C₁-C₆ of alkyl, alkyl alcohol, alkyl amine (including primary, secondary, tertiary amine, or quaternary ammonium) or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or pharmaceutical salts;

In another specific embodiment, a conjugate of a cell-binding molecule-camptothecin analog has the Formula (IV):

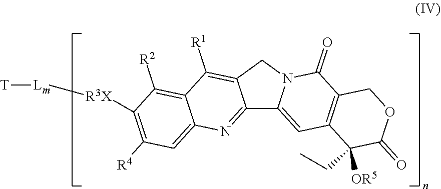

or their pharmaceutically acceptable salts, hydrates, or hydrated salts; or the polymorphic crystalline structures of these compounds; or their isotopes, optical isomers, racemates, diastereomers or enantiomers thereof;

Wherein T is a targeting or binding ligand; L is a releasable linker; n is 1-30 and m is 1-10;

Inside the bracket is a potent camptothecin analog wherein:

R¹ and R² are independently H, NR⁶R^{6′}, —N⁺R⁶R^{6′}R^{6″}, OH, SH, linear or branched C₁-C₆ of alkyl, alkyloxyl, alkyl amino (including primary, secondary, tertiary amino, or quaternary ammonium), oxylcarbonyl, aminocarbonyl, aminoalkyl, oxylalkyl, aminoalkylamino, oxylalkylamino, aminoalkyloxyl, oxylalkyloxyl, or alkyl carboxylic; C₂-C₆ of heteroalkyl, alkylcycloalkyl, heterocycloalkyl, heterocyclic, oxylcycloalkyl, aminocycloalkyl, heteroalkylcycloalkyl, alkylcarbonyl, aminoalkylcarbonyl, oxylalkylcarbonyl, alkyl ether, alkyl ester, alkyl amide, oxylalkylether, aminoalkylether, oxylalkylester, aminoalkylester, oxylalkylamide, aminoalkylamide, oxime; NH2, or OH;

R³ is independently —NHC(O)—, —C(O)—, SO₂—, —SO₂NH—, —NR⁶SO₂—, R⁶NHC(O), R⁶OC(O), SO₂R⁶, SO₃R⁶, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR^{6′})₂, R⁶C(O), C(O)N R⁶R^{6′}; linear or branched C₁-C₆ of alkyl, alkyl alcohol, alkyl amine (including primary, secondary, tertiary amine), or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide, oxime;

R⁴ is halo (F, Cl, Br, or I), CN, NO₂, SO₃H, OR⁶, SR⁶, S(O₂)R⁶, NH(R⁶)S(O₂)R⁶, N(R⁶)(R^{6′}), C(O)XR⁶, N+(R⁶)(R^{6′})(R^{6″});

X is NH or O;

R⁵ is H, C(O)OR⁶, C(O)NHR⁶, R⁶C(O), linear or branched C₁-C₆ of alkyl, alkyl alcohol, alkyl amine (including primary, secondary, tertiary amine, or quaternary ammonium), alkyl carboxylic acid; C₂-C₆ of carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;

R⁶, R^{6′}, and R^{6″} are independently H, C₁-C₆ of alkyl, alkyl alcohol, alkyl amine (including primary, secondary, tertiary amine, or quaternary ammonium) or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or pharmaceutical salts;

In addition, R³ can be absent and X of C-10 directly links to L, and R², R³, X, C-10 and C-9 can join together to form a 5-, 6- or 7-member heterocyclic ring.

Illustrative compounds inside the bracket of Formula (IV) have the structures:

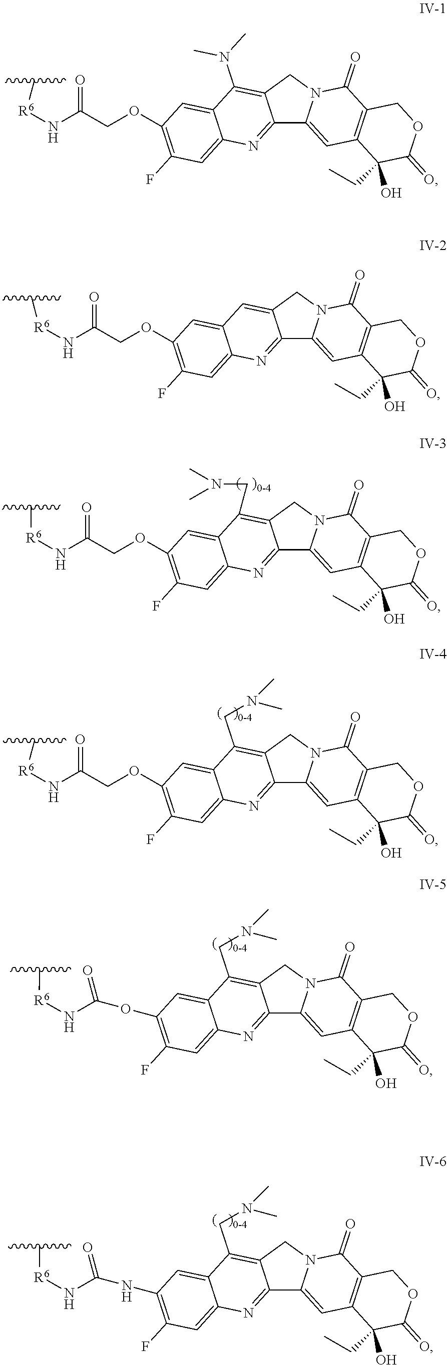

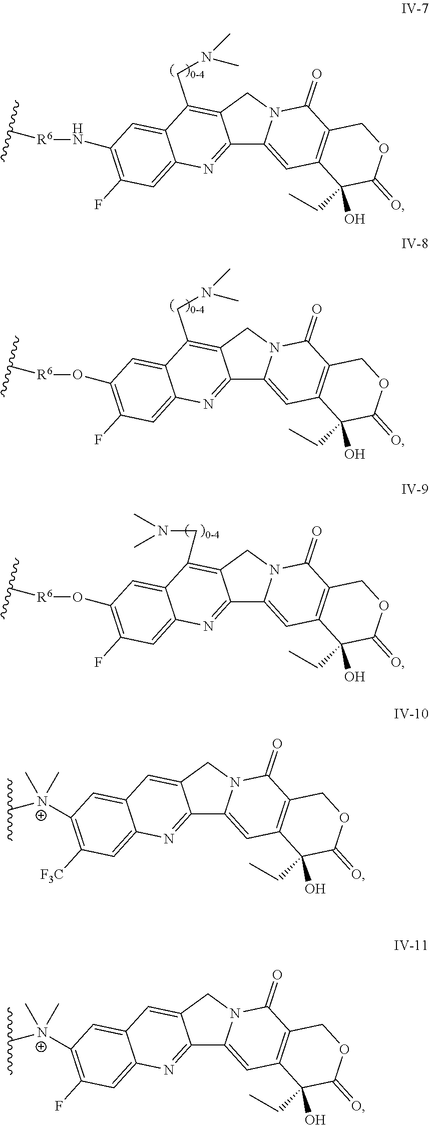

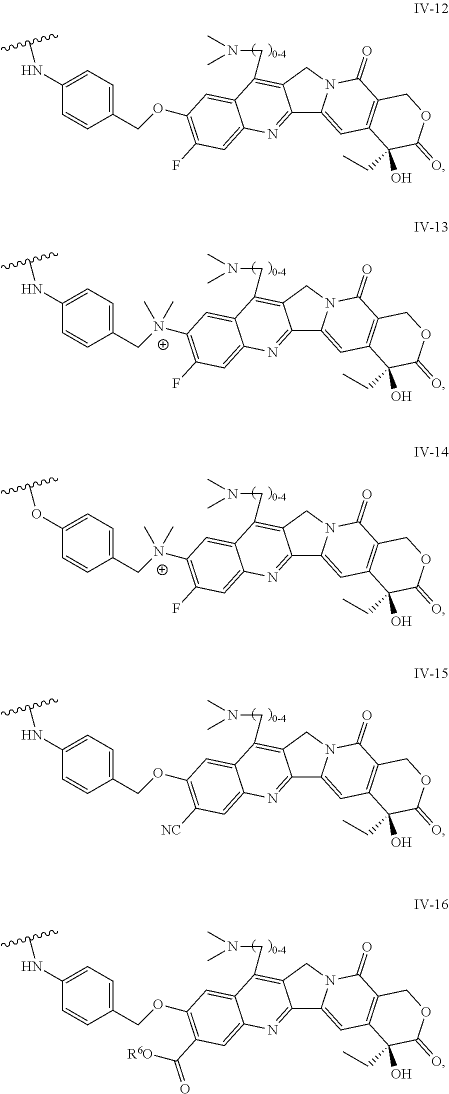

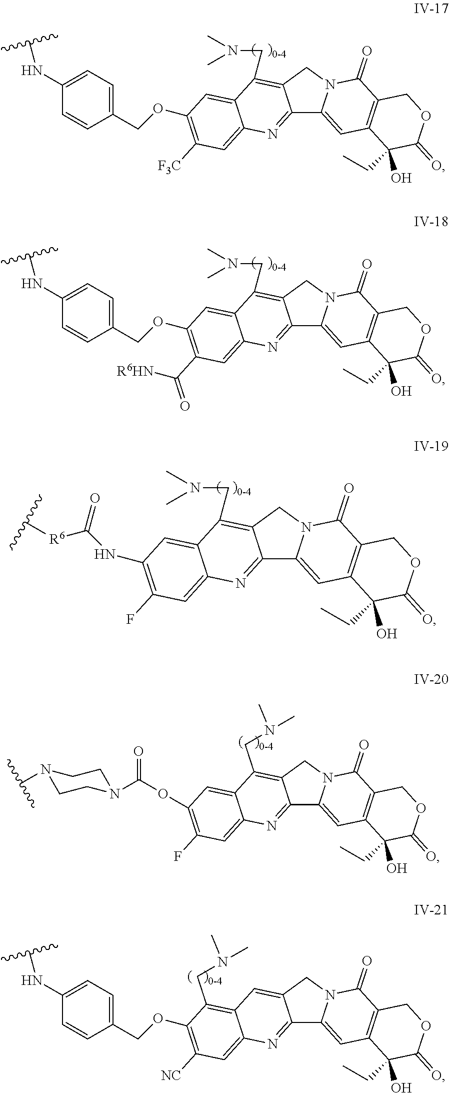

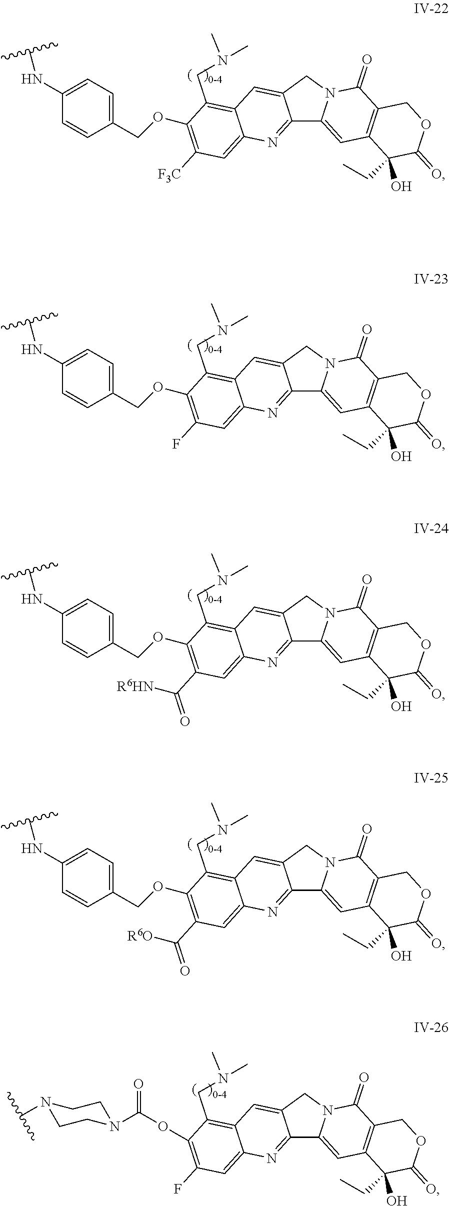

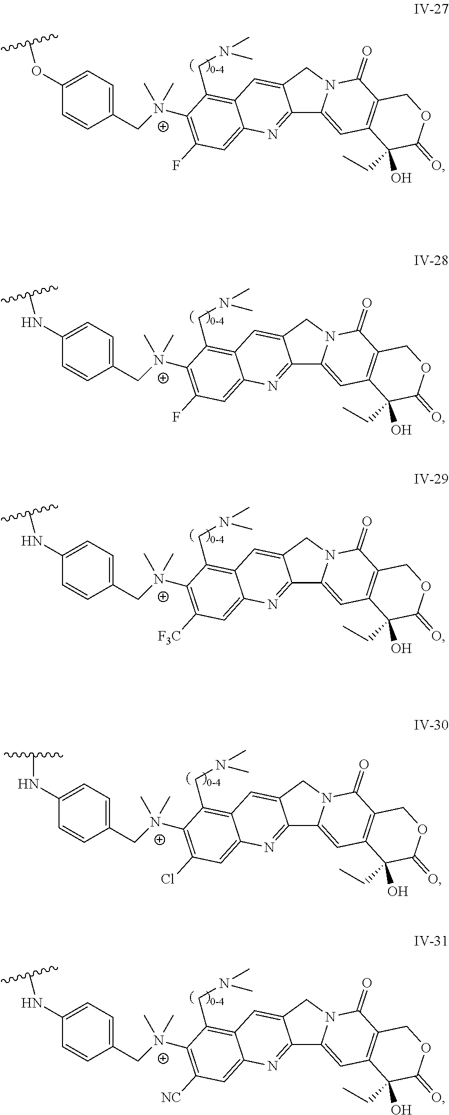

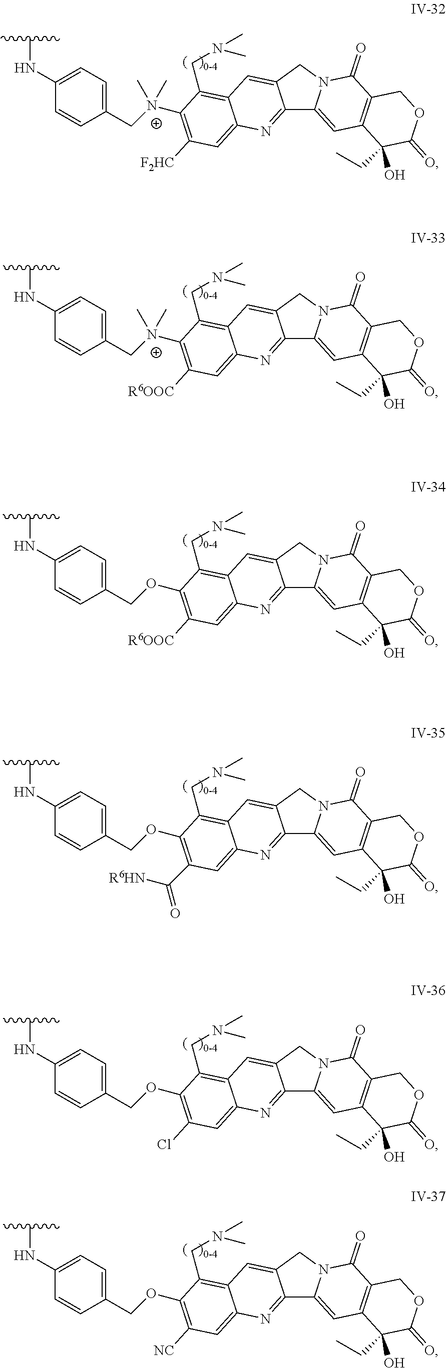

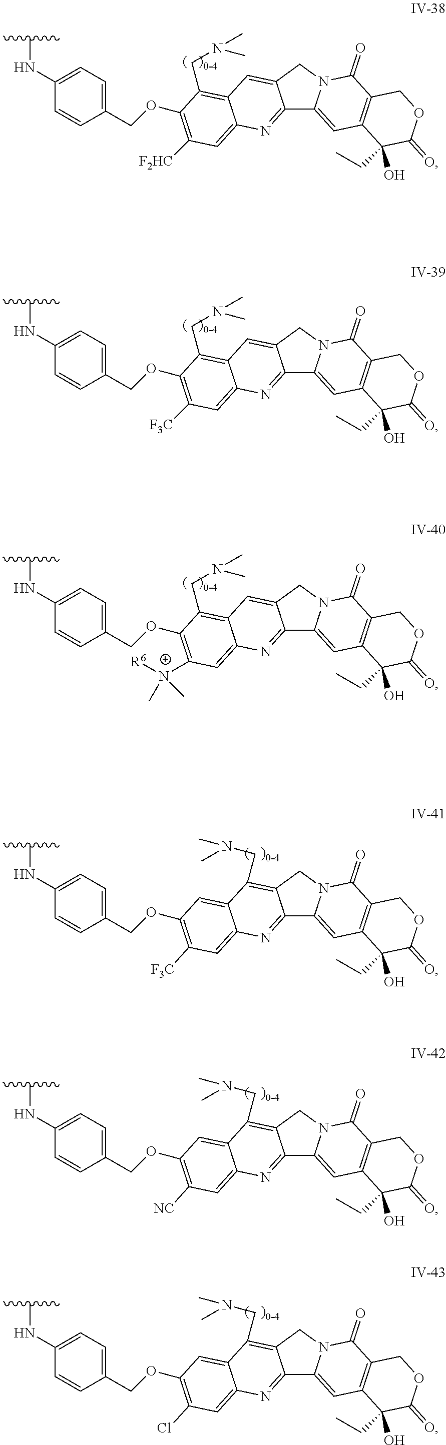

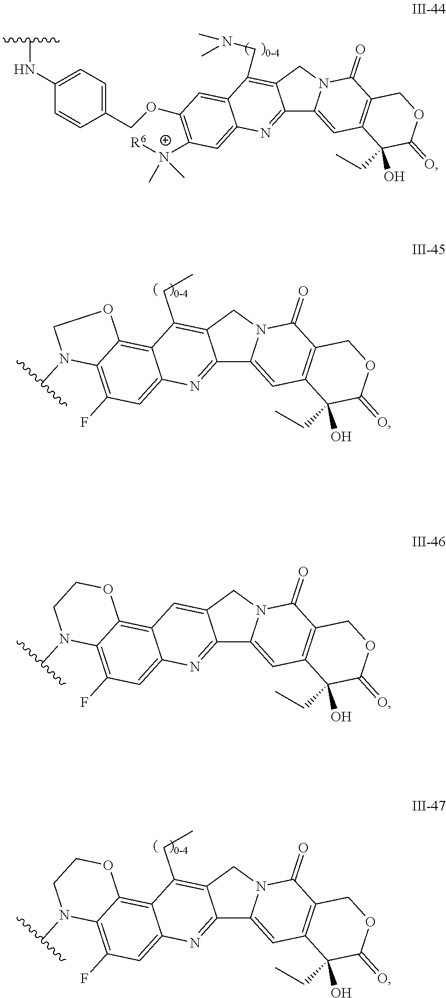

or their pharmaceutically acceptable salts, hydrates, or hydrated salts; or the polymorphic crystalline structures of these compounds; or their isotope, optical isomers, racemates, diastereomers or enantiomers; wherein “ [symbol] ”, R⁶, and R^{6′}, are defined the same as above;

In another specific embodiment, a conjugate of a cell-binding molecule-camptothecin analog has the Formula (V):

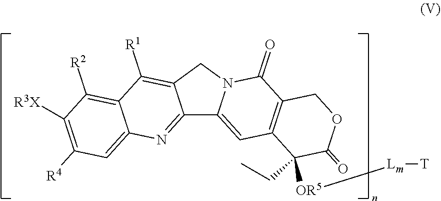

or their pharmaceutically acceptable salts, hydrates, or hydrated salts; or the polymorphic crystalline structures of these compounds; or their isotopes, optical isomers, racemates, diastereomers or enantiomers thereof;

Wherein T is a targeting or binding ligand; L is a releasable linker; n is 1-30 and m is 1-10;

Inside the bracket is a potent camptothecin analog, wherein:

R¹ and R² are independently H, NR⁶R^{6′}, —N⁺R⁶R^{6′}R^{6″}, OH, SH, linear or branched C₁-C₆ of alkyl, alkyloxyl, alkyl amino (including primary, secondary, tertiary amino, or quaternary ammonium), oxylcarbonyl, aminocarbonyl, aminoalkyl, oxylalkyl, aminoalkylamino, oxylalkylamino, aminoalkyloxyl, oxylalkyloxyl, or alkyl carboxylic; C₂-C₆ of heteroalkyl, alkylcycloalkyl, heterocycloalkyl, heterocyclic, oxylcycloalkyl, aminocycloalkyl, heteroalkylcycloalkyl, alkylcarbonyl, aminoalkylcarbonyl, oxylalkylcarbonyl, alkyl ether, alkyl ester, alkyl amide, oxylalkylether, aminoalkylether, oxylalkylester, aminoalkylester, oxylalkylamide, aminoalkylamide, oxime; NH2, or OH;

R³ is independently R⁶NHC(O)—, R⁶C(O)—, R⁶SO₂, —SO₂NHR⁶, R⁶OC(O), R⁶'SO₂R⁶—, SO₃R⁶, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR^{6′})₂, R⁶C(O), C(O)N R⁶R^{6′}; linear or branched C₁-C₆ of alkyl, alkyl alcohol, alkyl amine (including primary, secondary, tertiary amine), or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide, oxime;

R⁴ is halo (F, Cl, Br, or I), CN, NO₂, SO₃H, OR⁶, SR⁶, S(O₂)R⁶, NH(R⁶)S(O₂)R^{6′}, N(R⁶)(R^{6′}), C(O)XR⁶, N+(R⁶)(R^{6′})(R^{6″});

X is NH or O;

R⁵ is C(O)O, C(O)NH, R⁶C(O), linear or branched C₁-C₆ of alkyl, alkyl alcohol, alkyl amine (including primary, secondary, tertiary amine, or quaternary ammonium), alkyl carboxylic acid; C₂-C₆ of carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;

R⁶, R^{6′}, and R^{6″} are independently H, C₁-C₆ of alkyl, alkyl alcohol, alkyl amine (including primary, secondary, tertiary amine, or quaternary ammonium) or alkyl carboxylic acid; C₂-C₆ of heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or pharmaceutical salts;

In addition, R⁵ can be absent and O of C-20 directly links to L, and R², R³, X, C-10 and C-9 can join together to form a 5-, 6- or 7-member heterocyclic ring.

Illustrative compounds inside the bracket of Formula (V) have the structures:

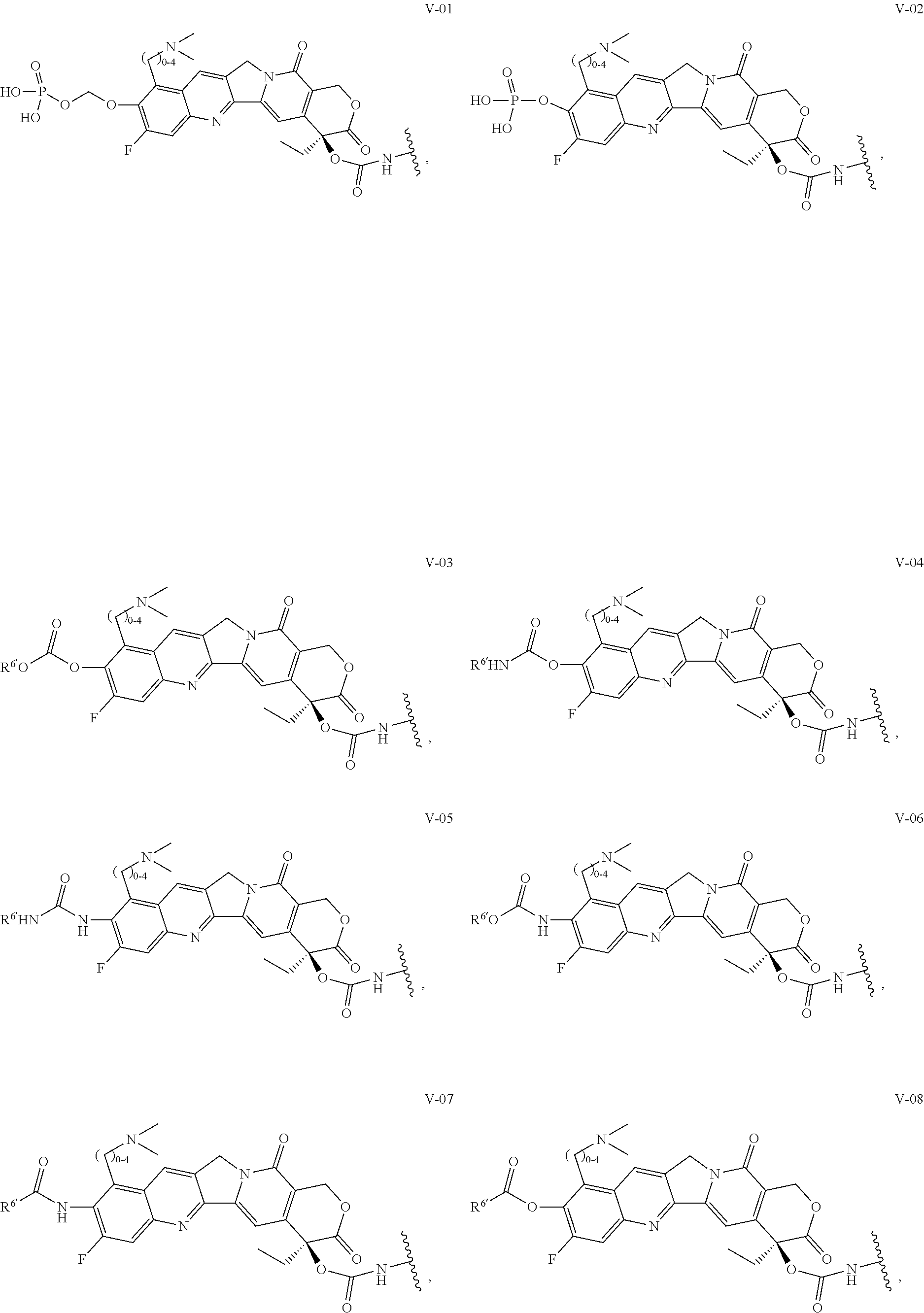

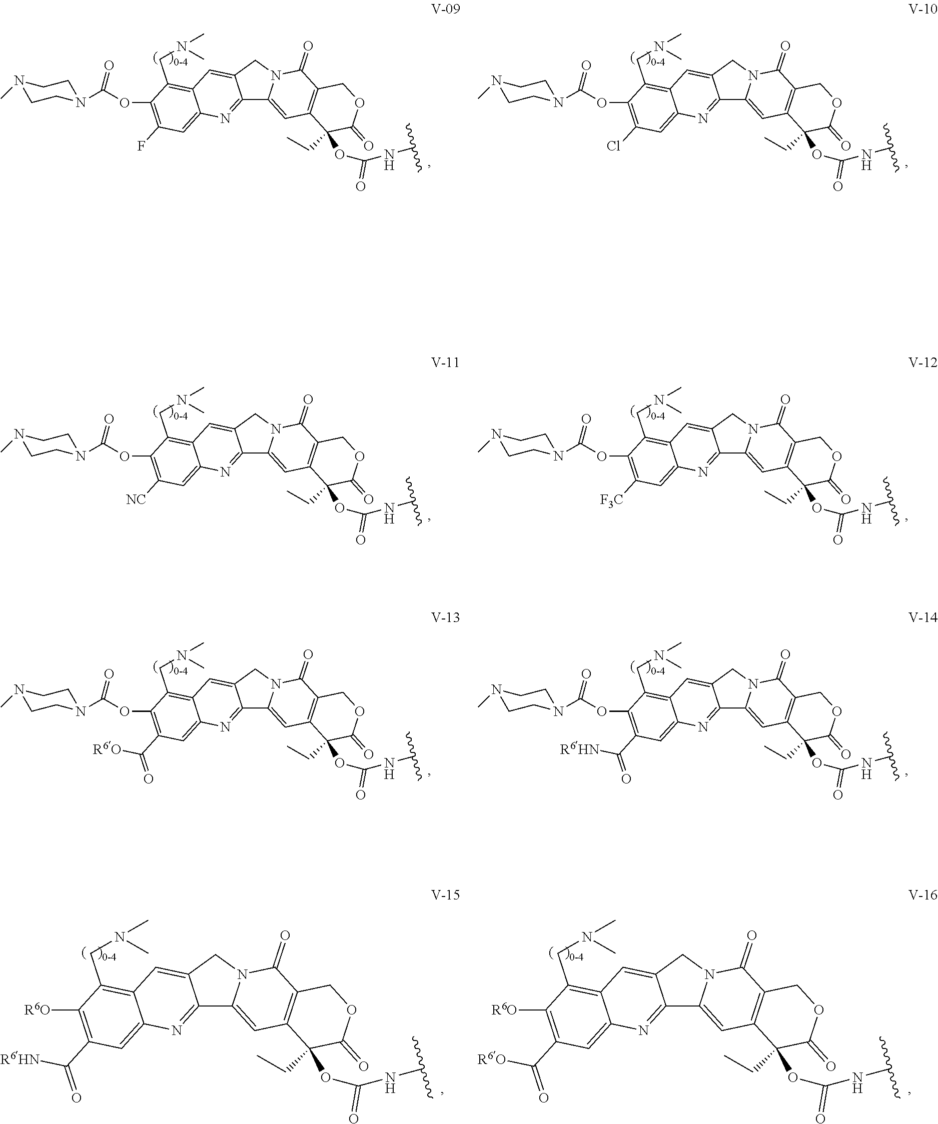

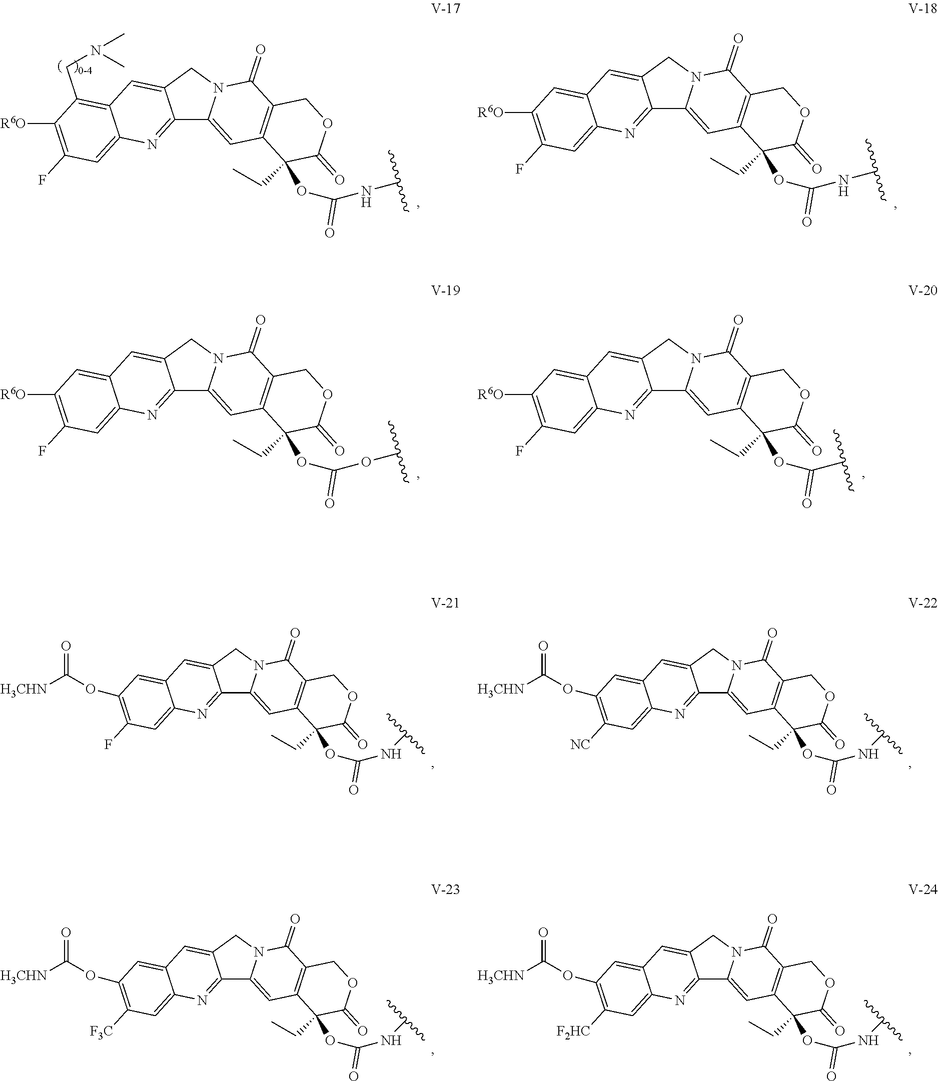

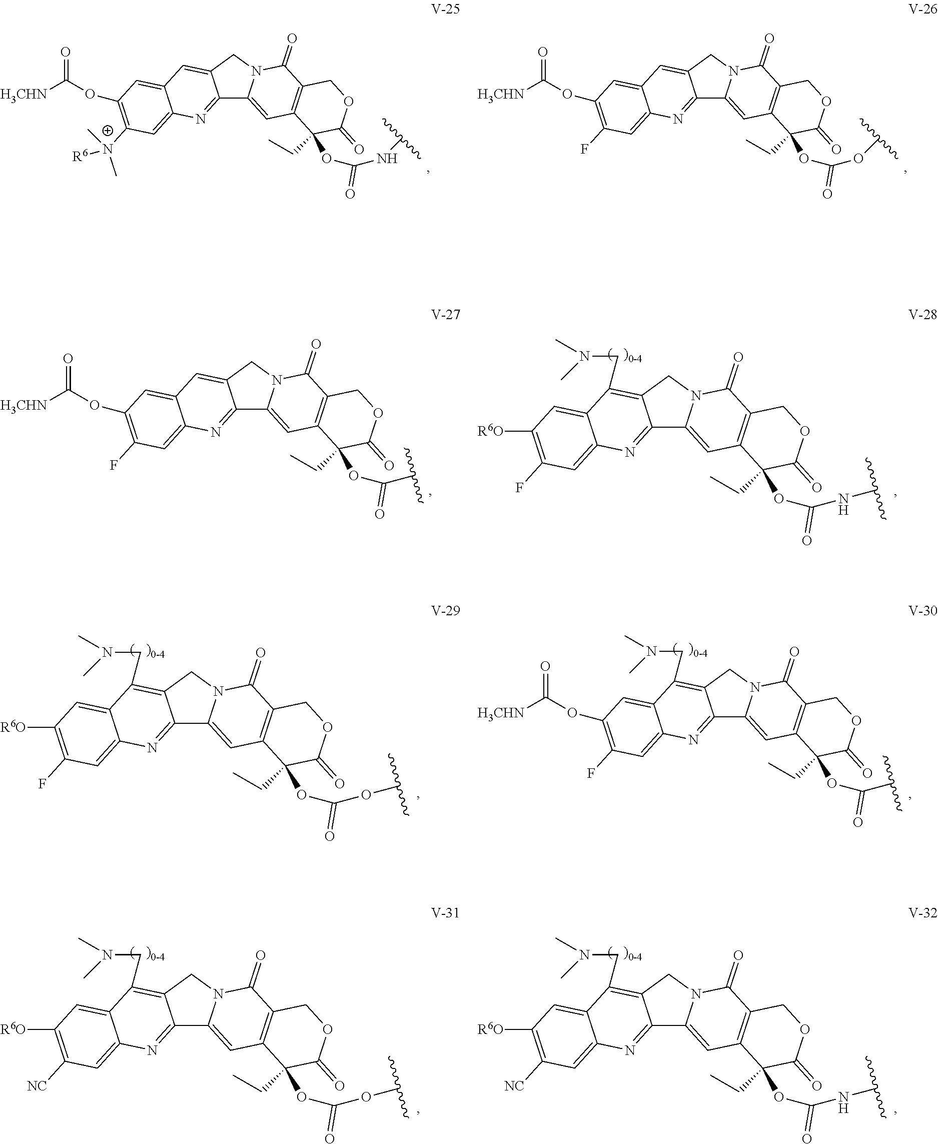

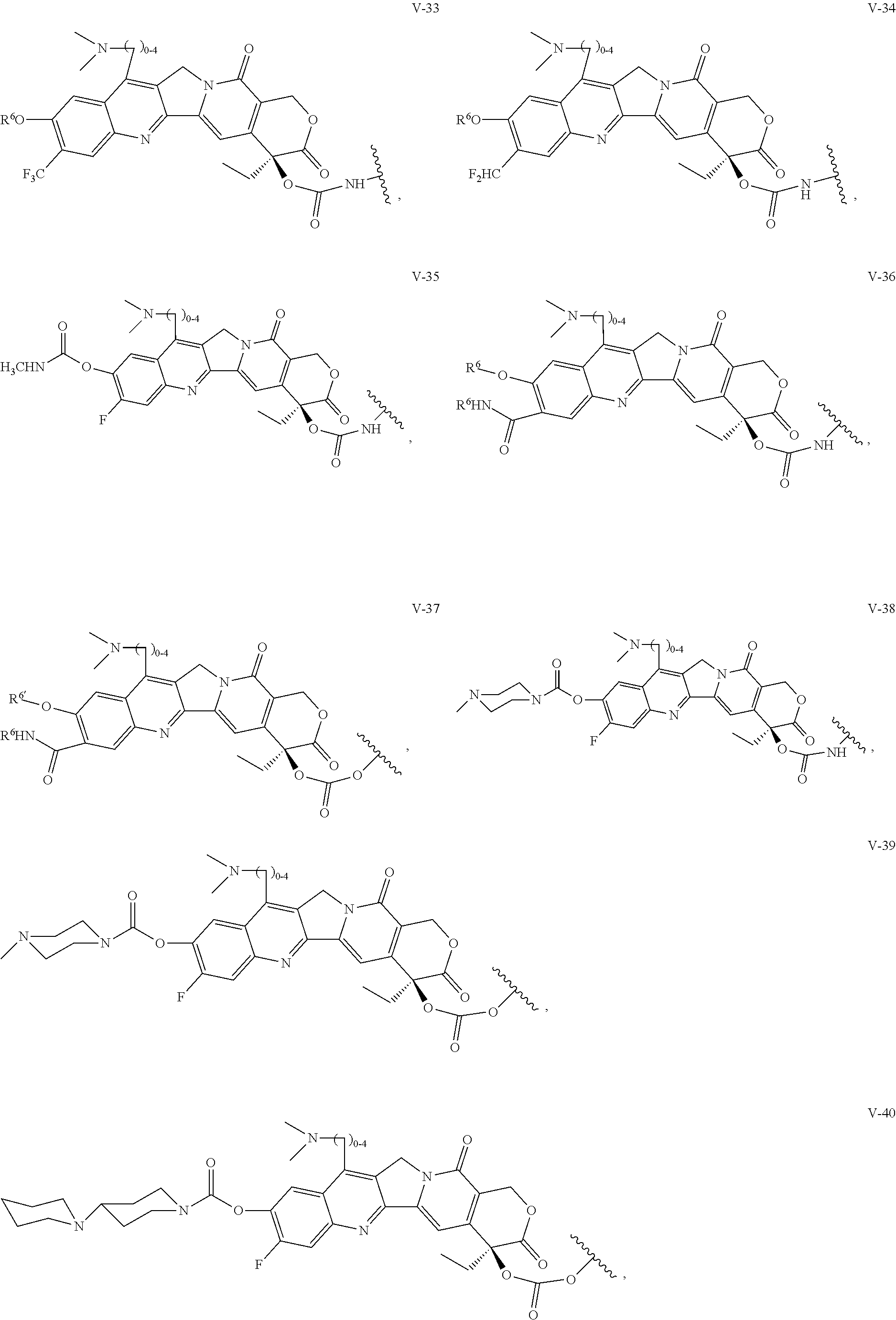

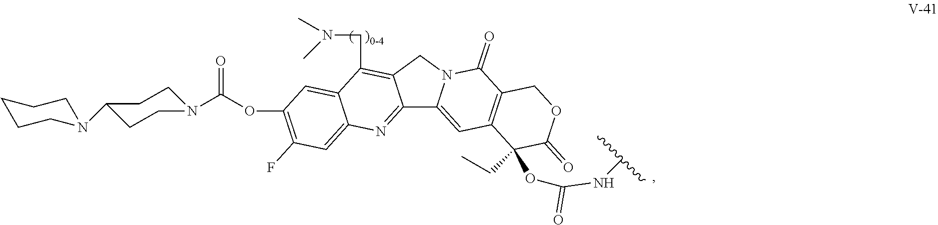

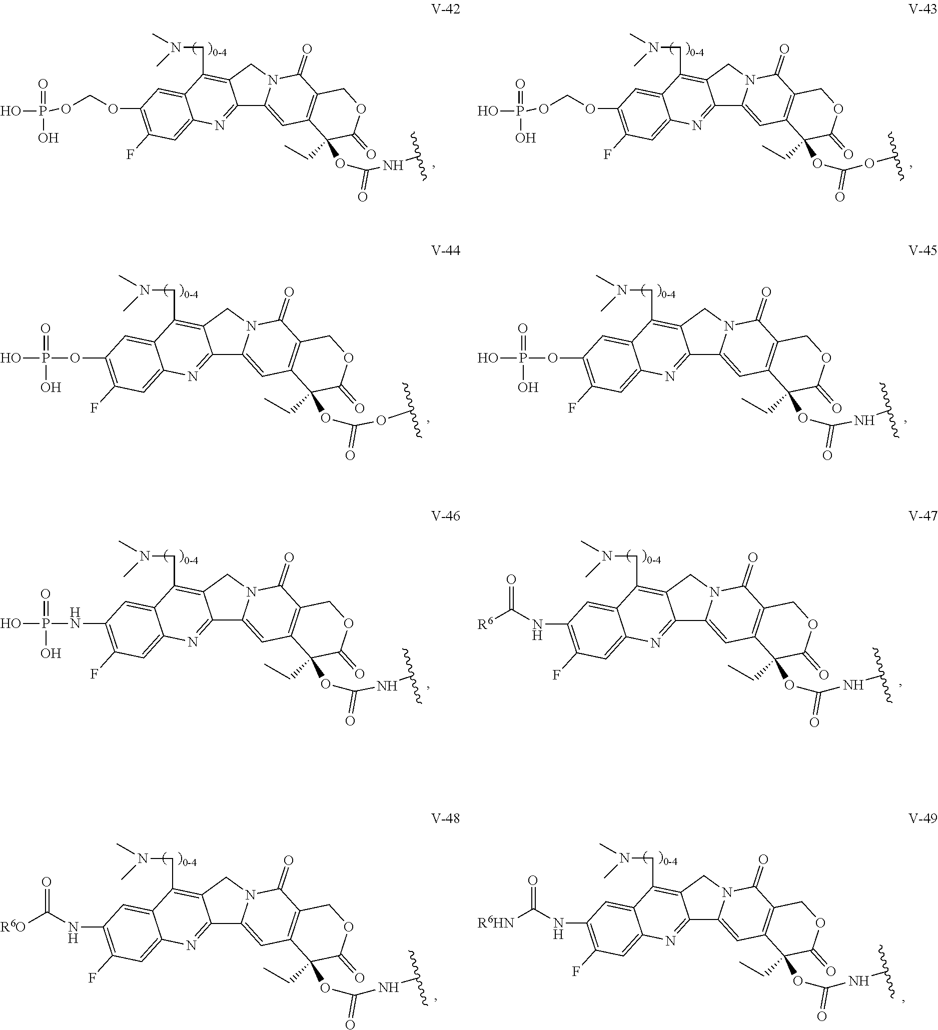

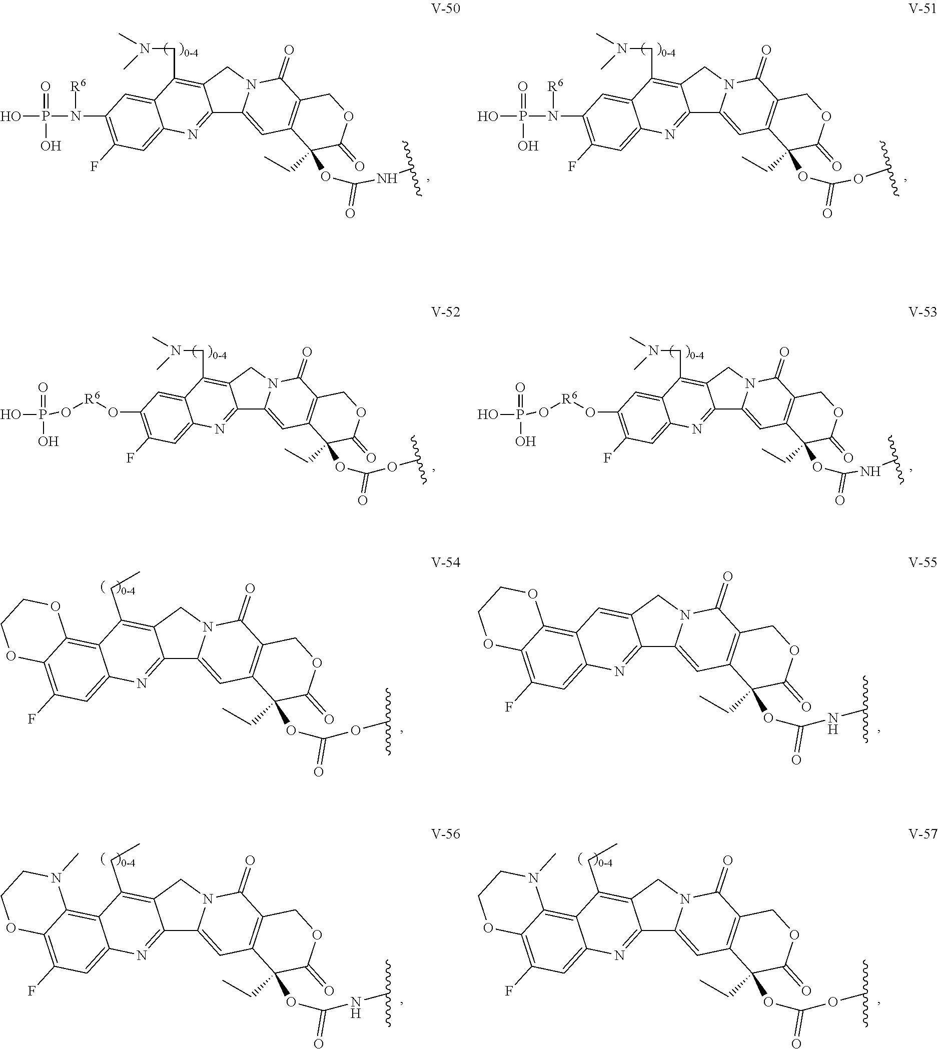

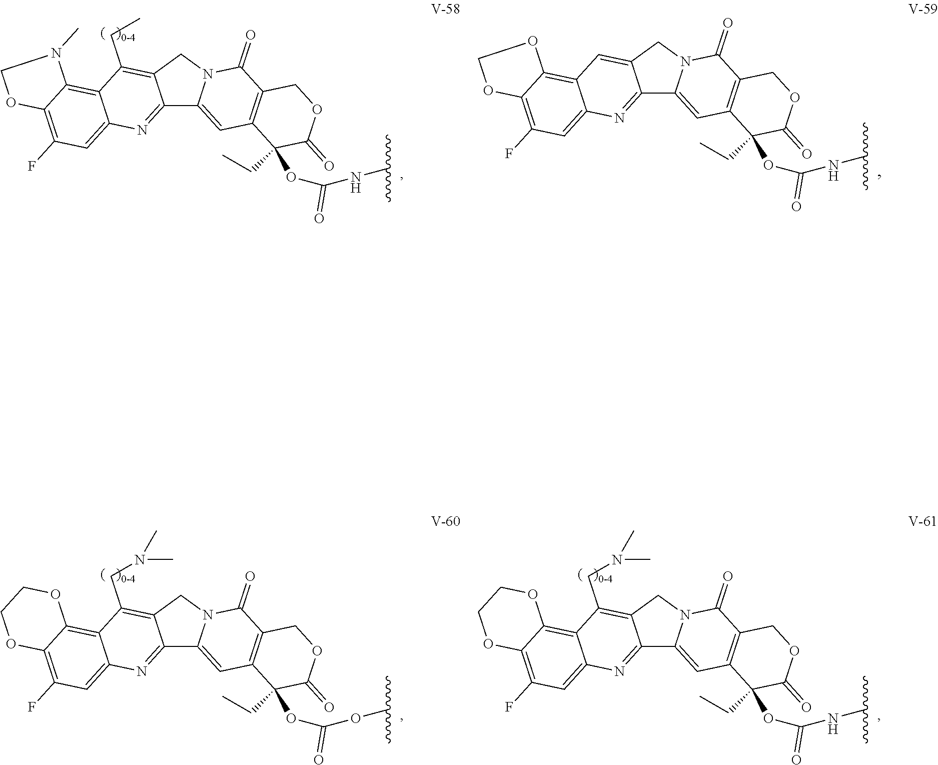

or their pharmaceutically acceptable salts, hydrates, or hydrated salts; or the polymorphic crystalline structures of these compounds; or their isotope, optical isomers, racemates, diastereomers or enantiomers; wherein “ [symbol] ”, R⁶, and R^{6′}, are defined the same as above;

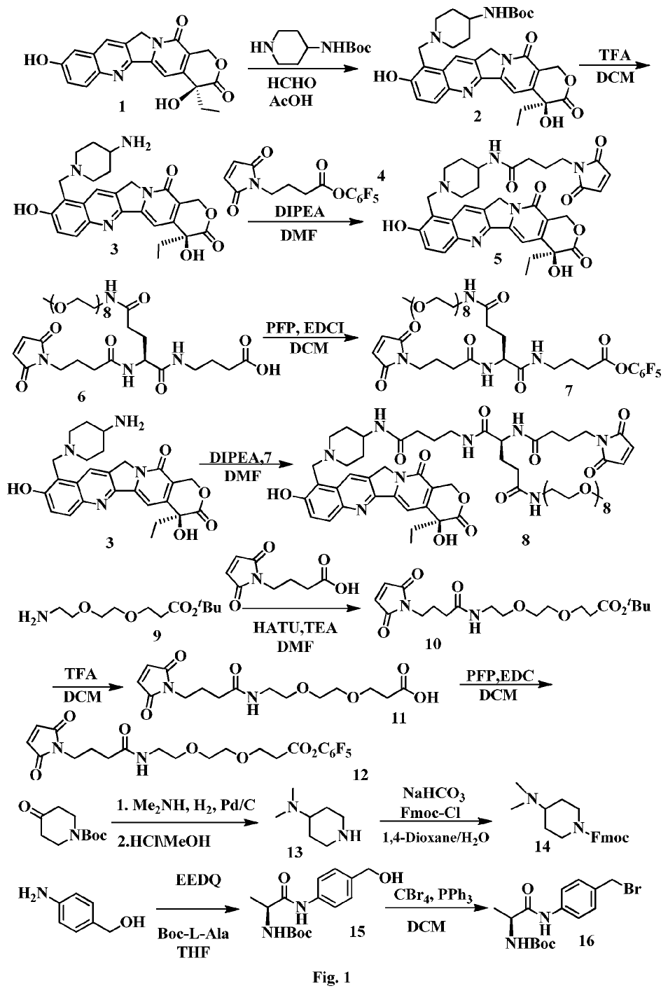

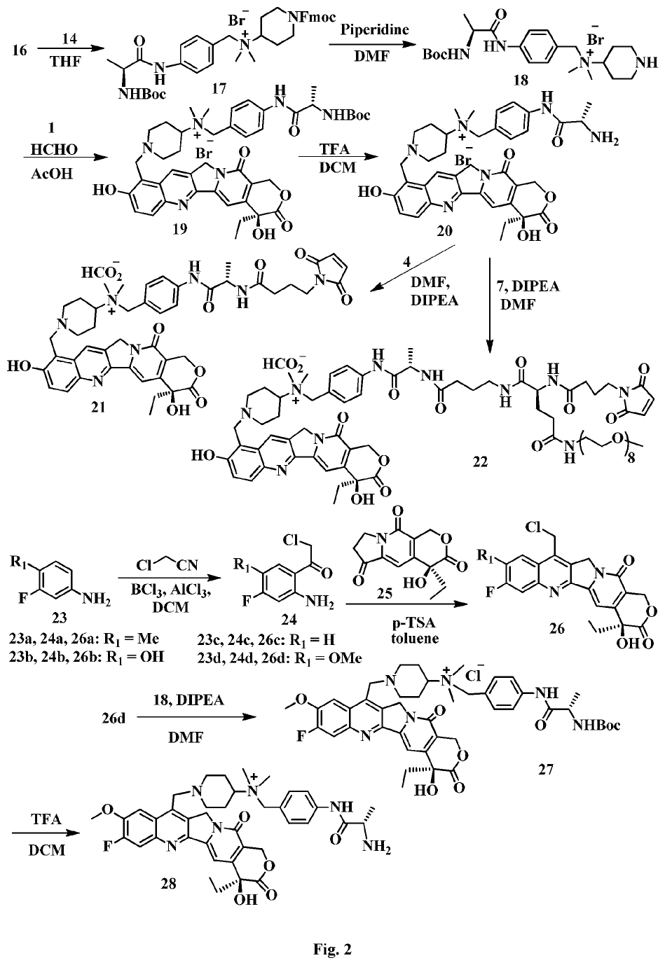

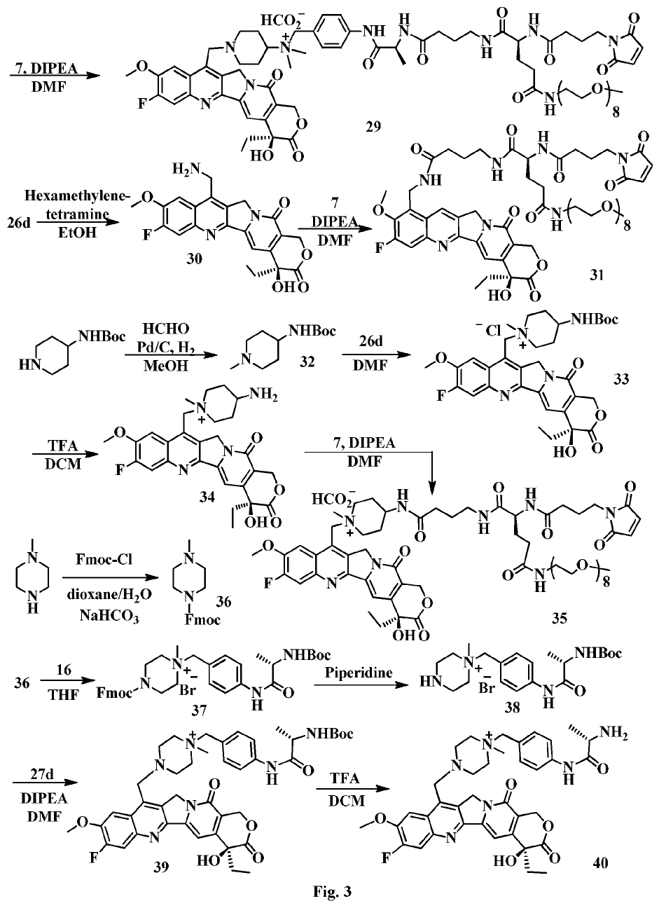

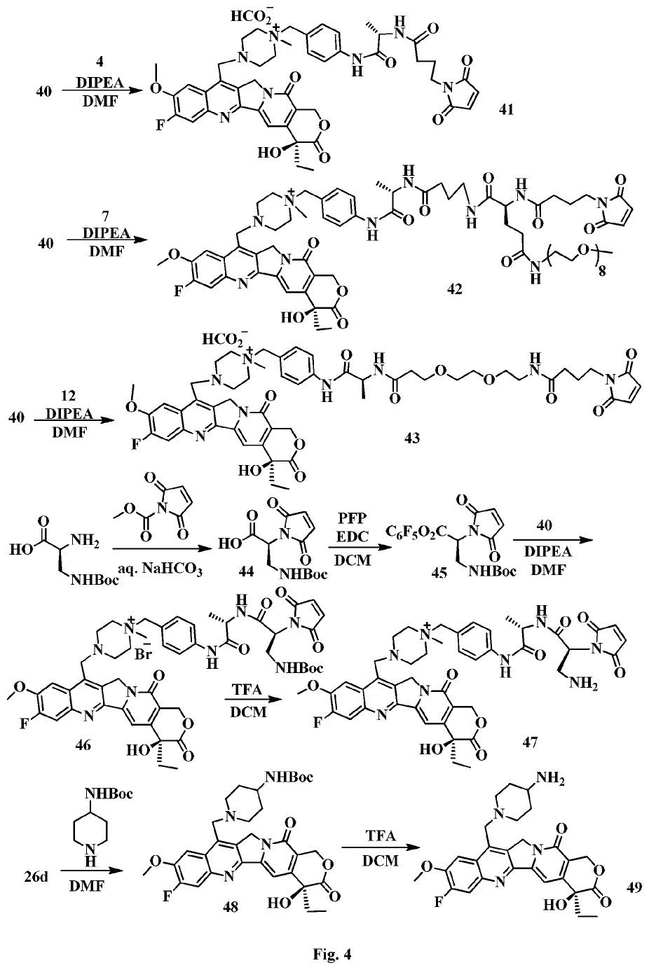

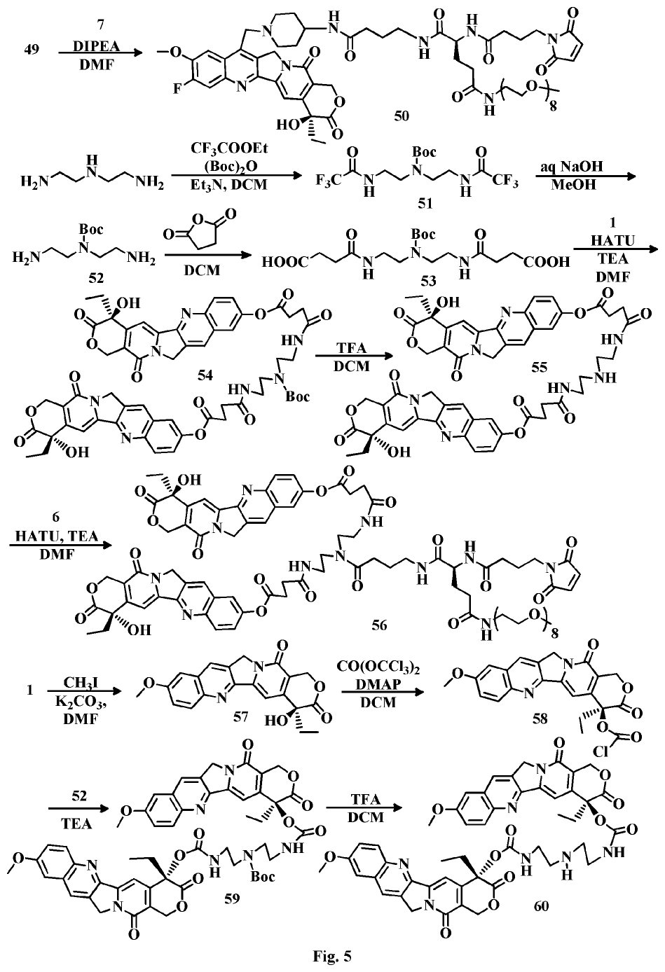

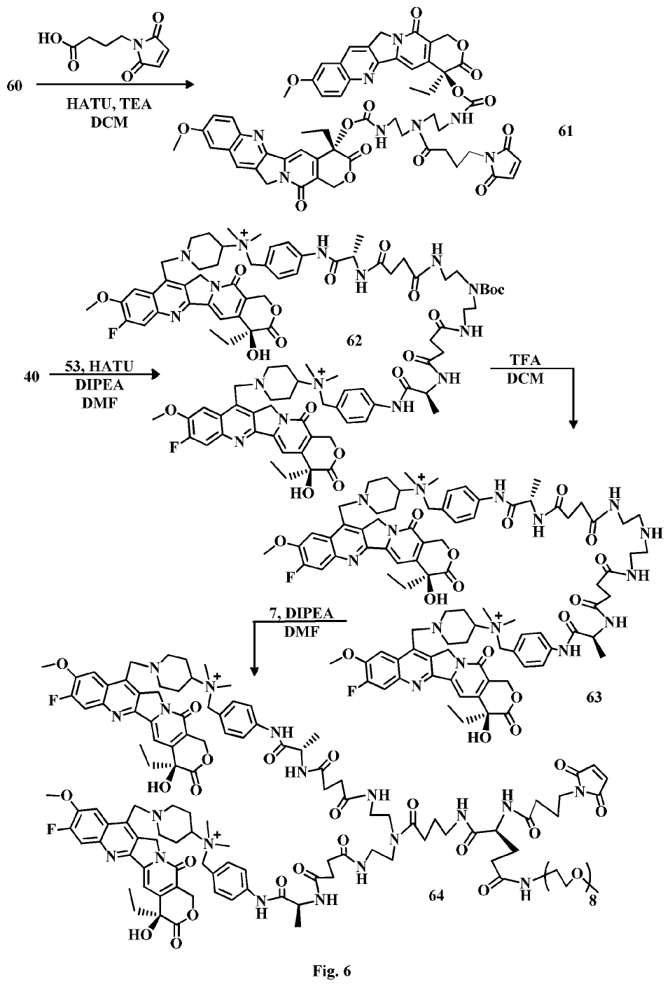

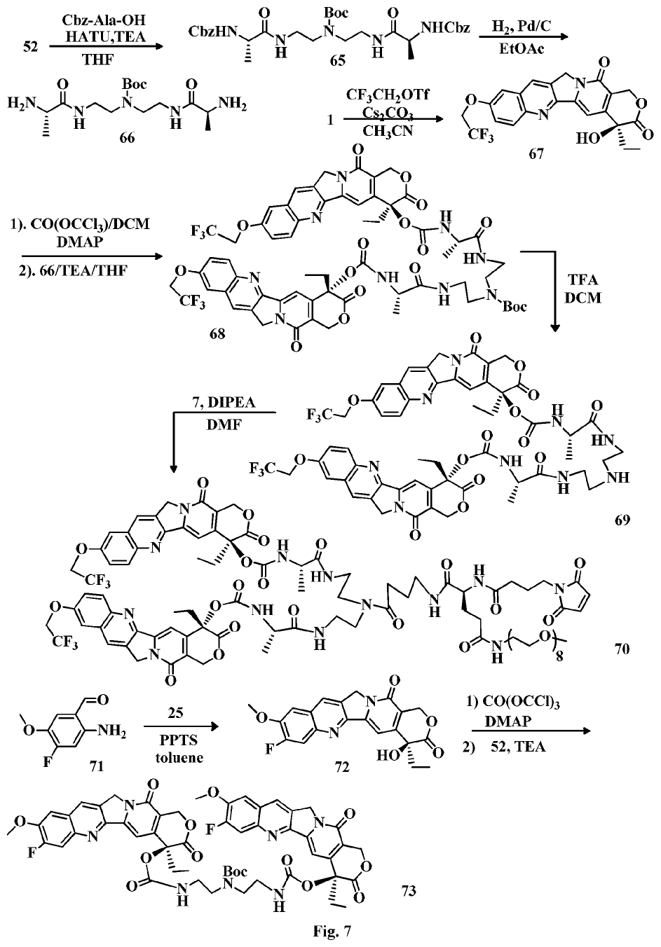

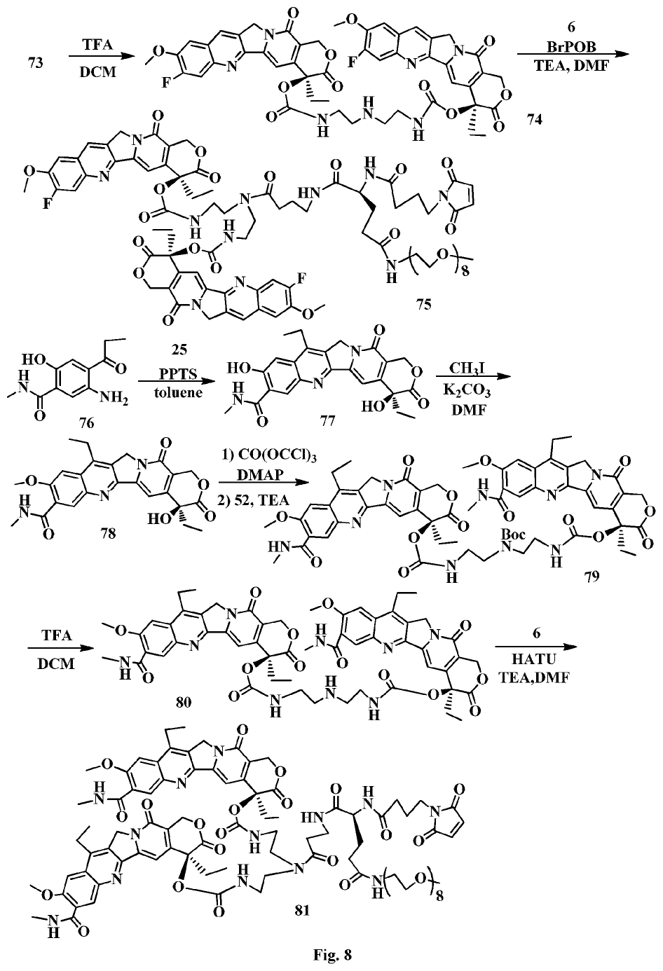

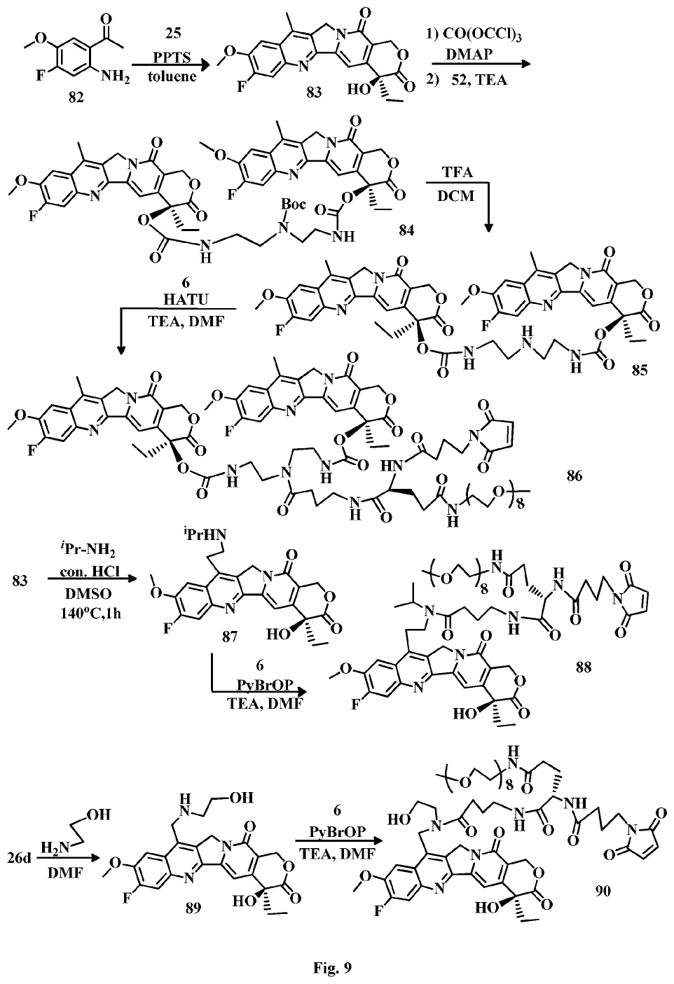

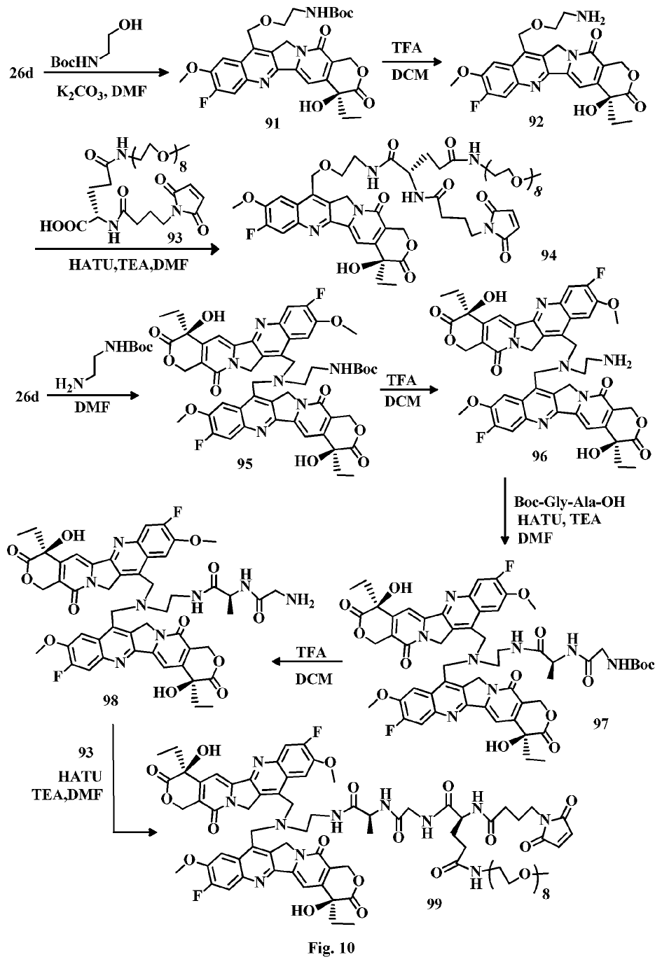

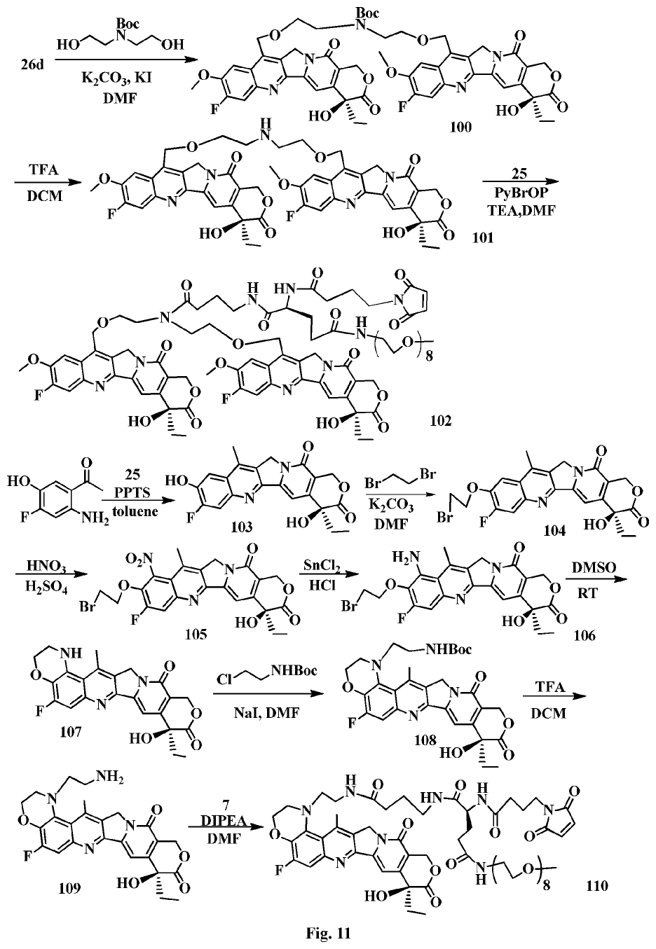

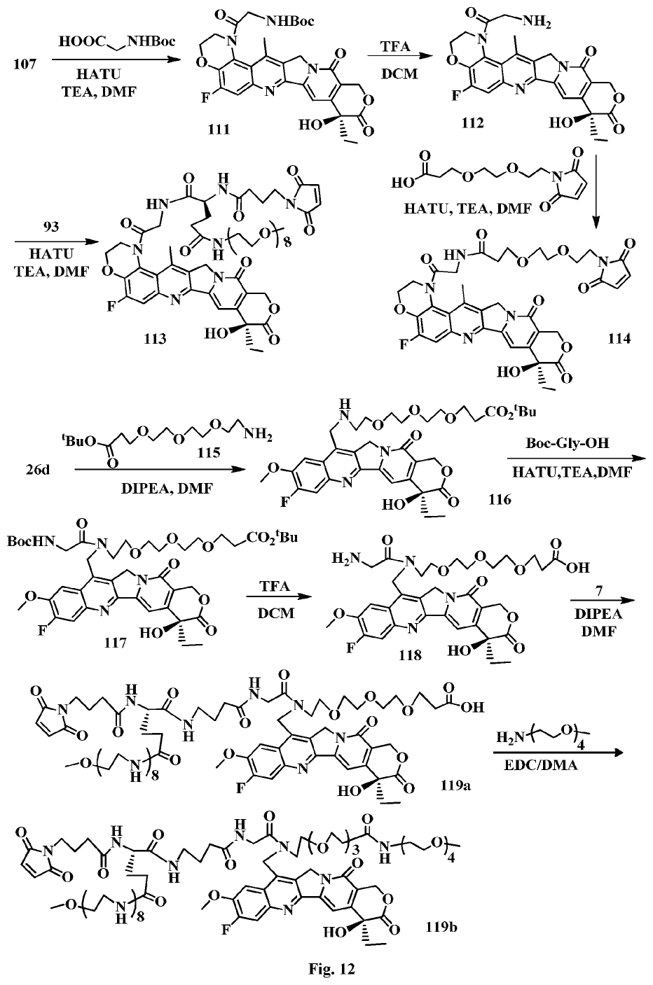

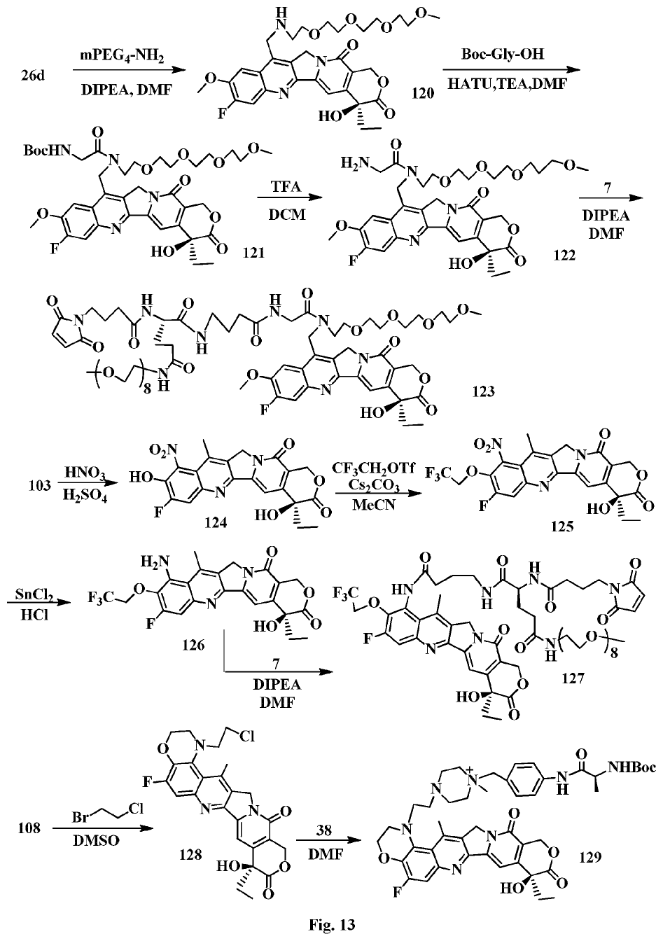

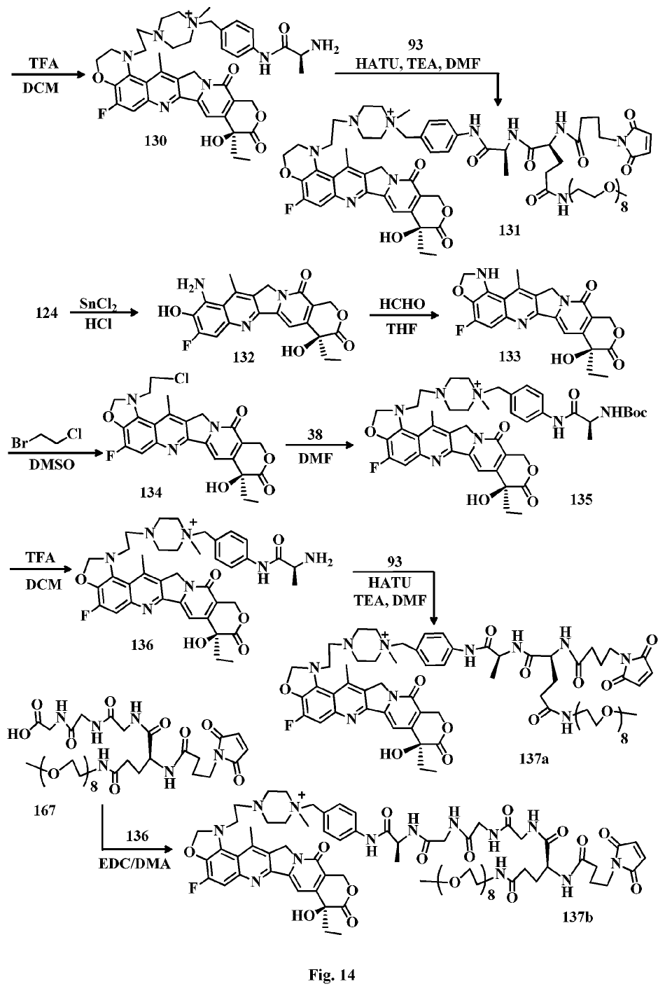

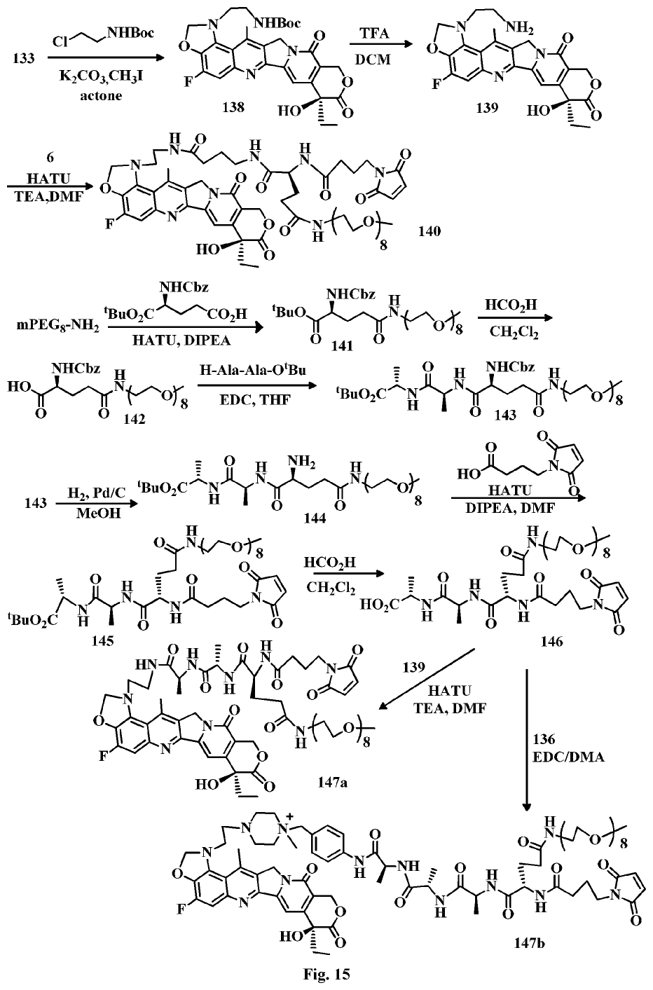

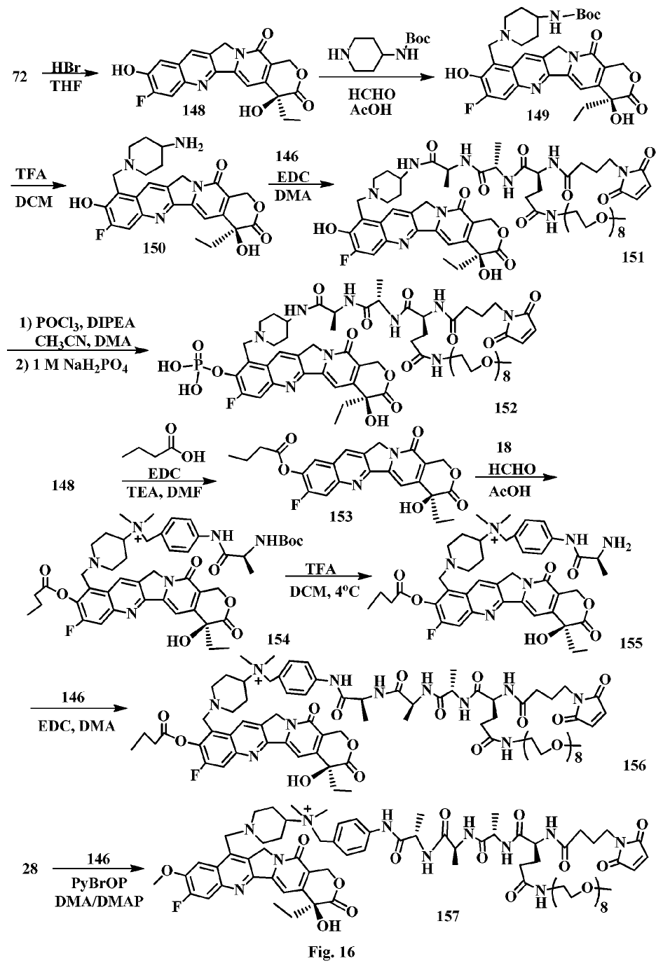

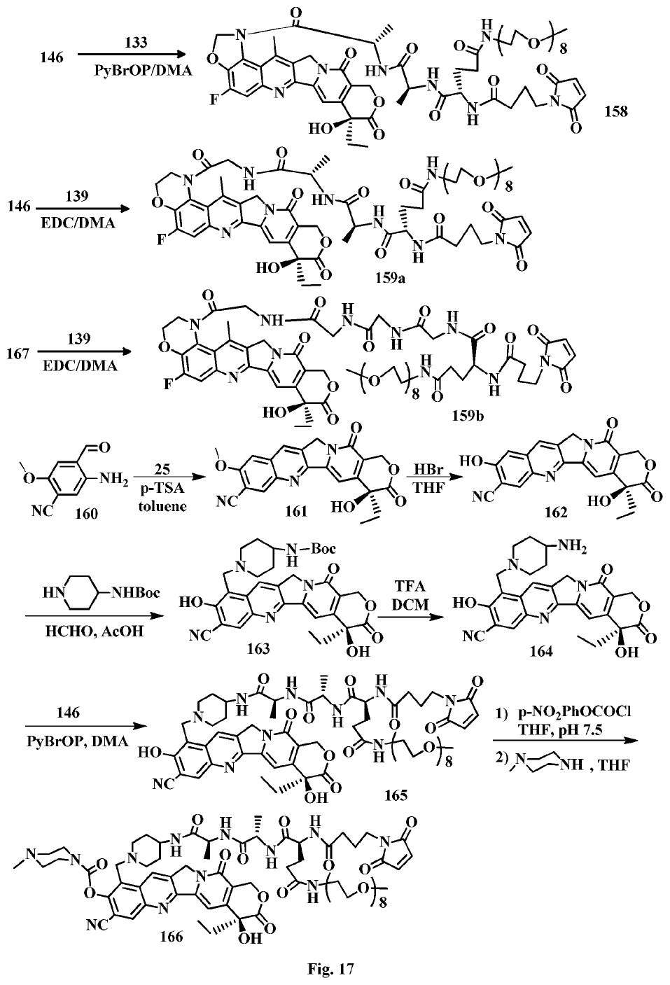

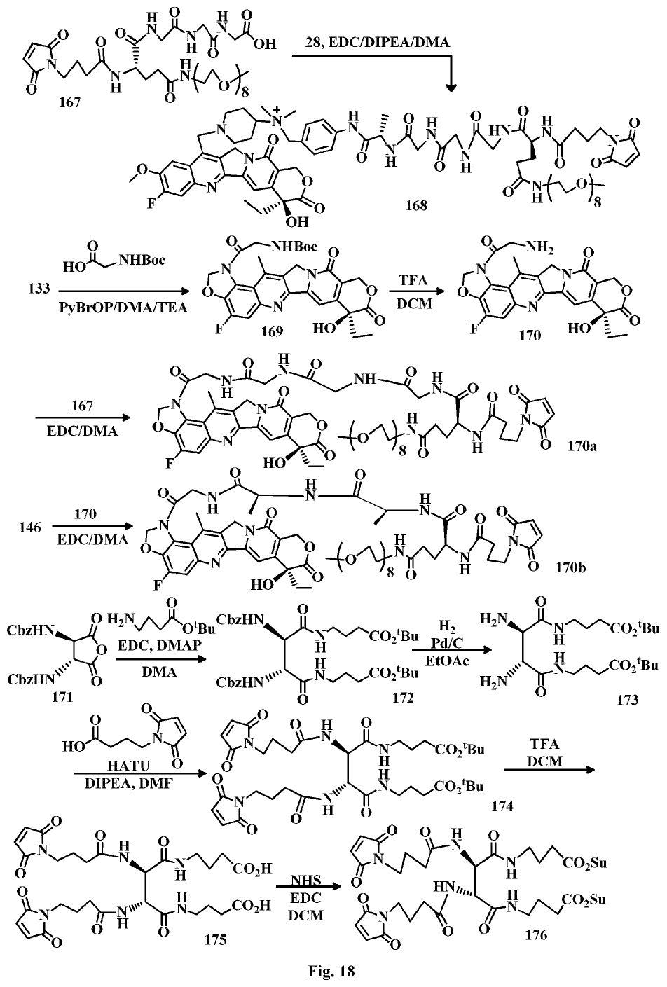

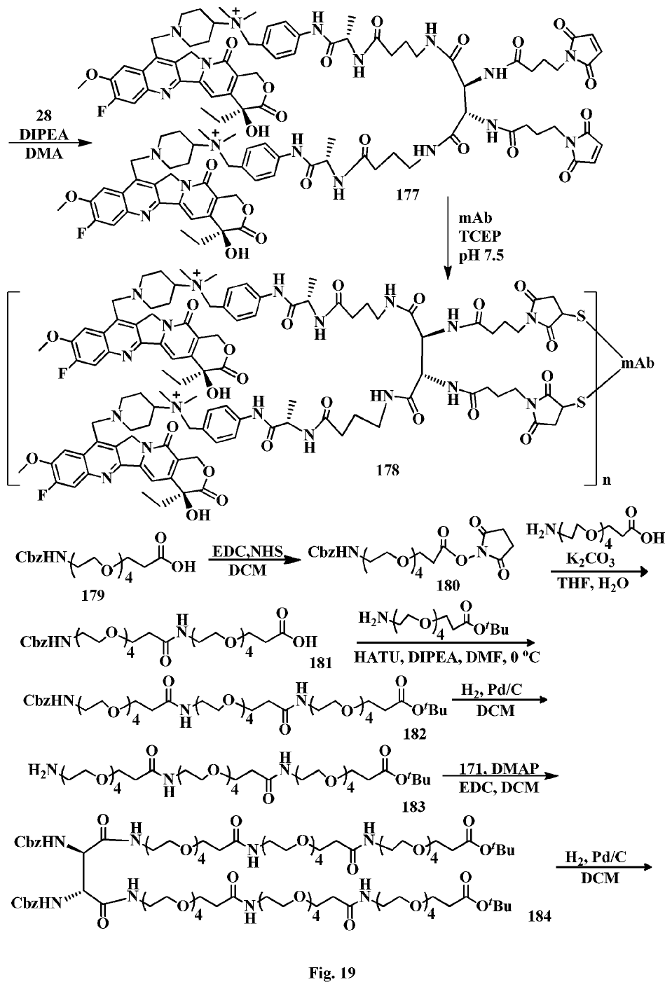

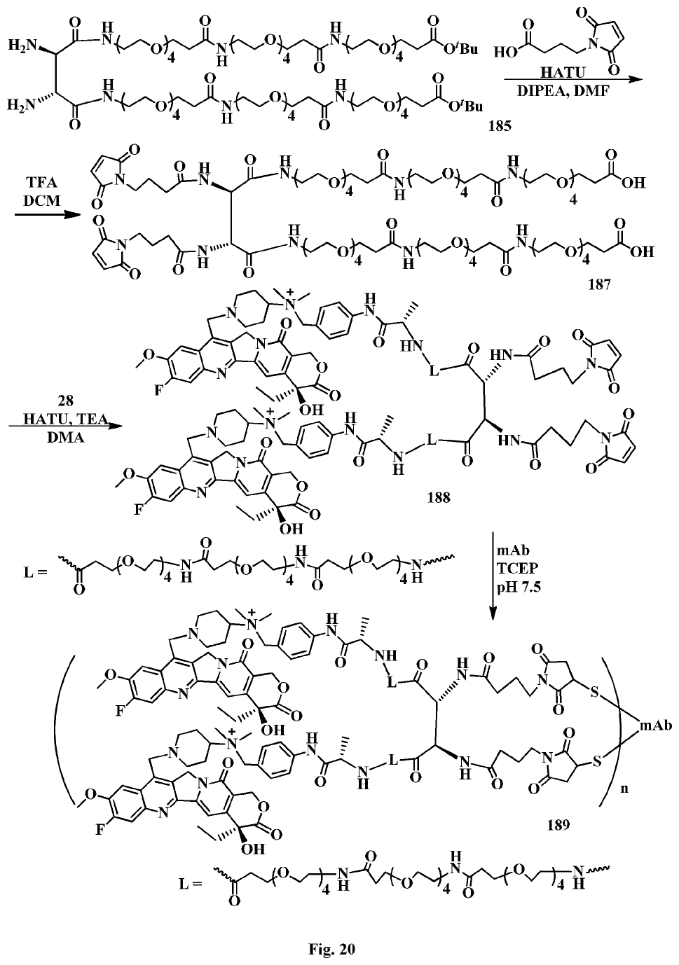

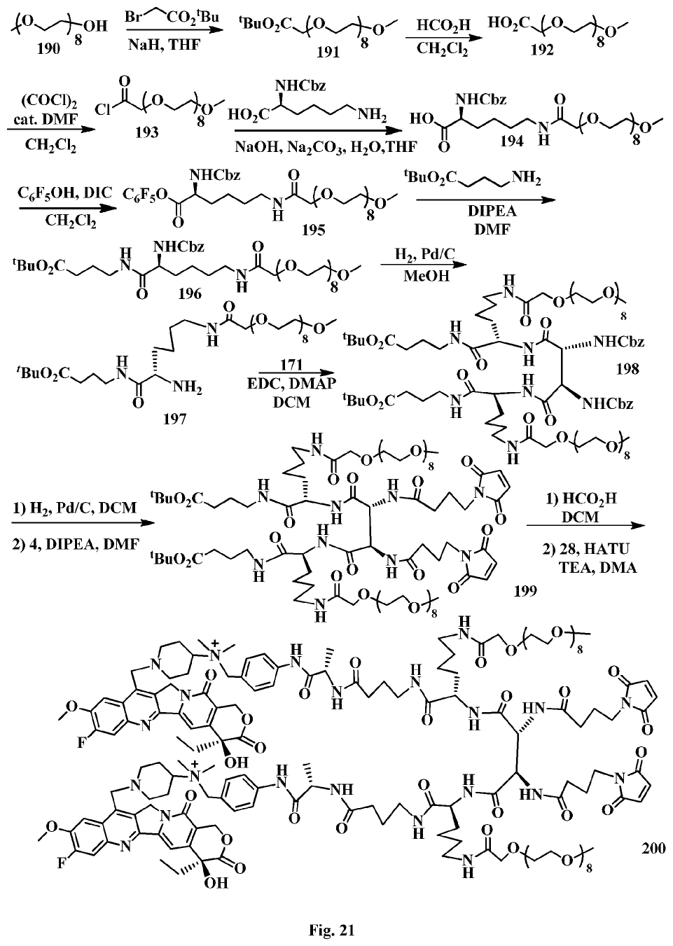

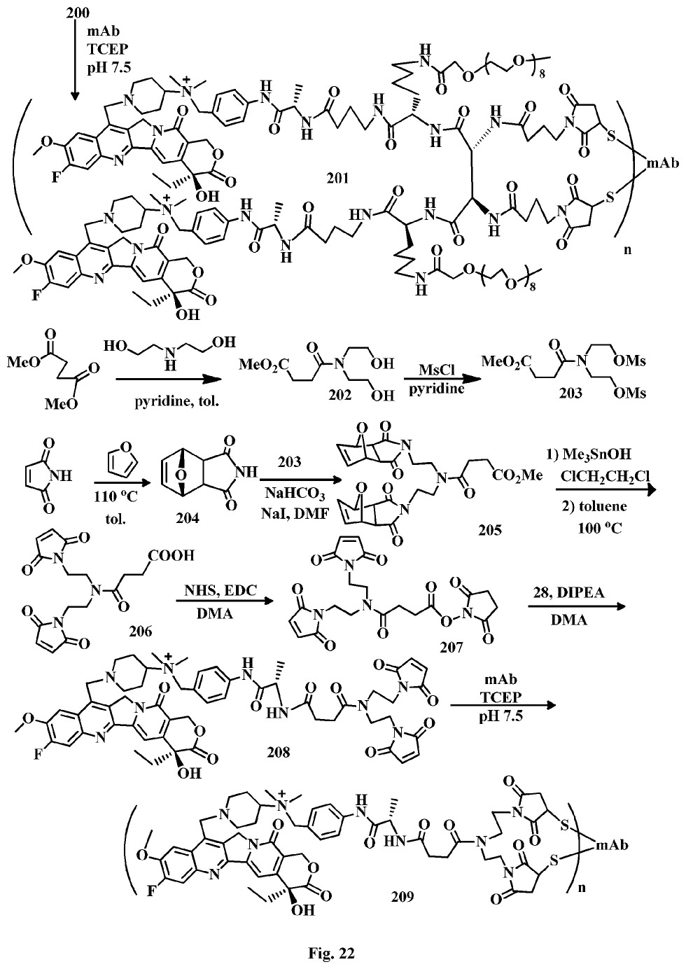

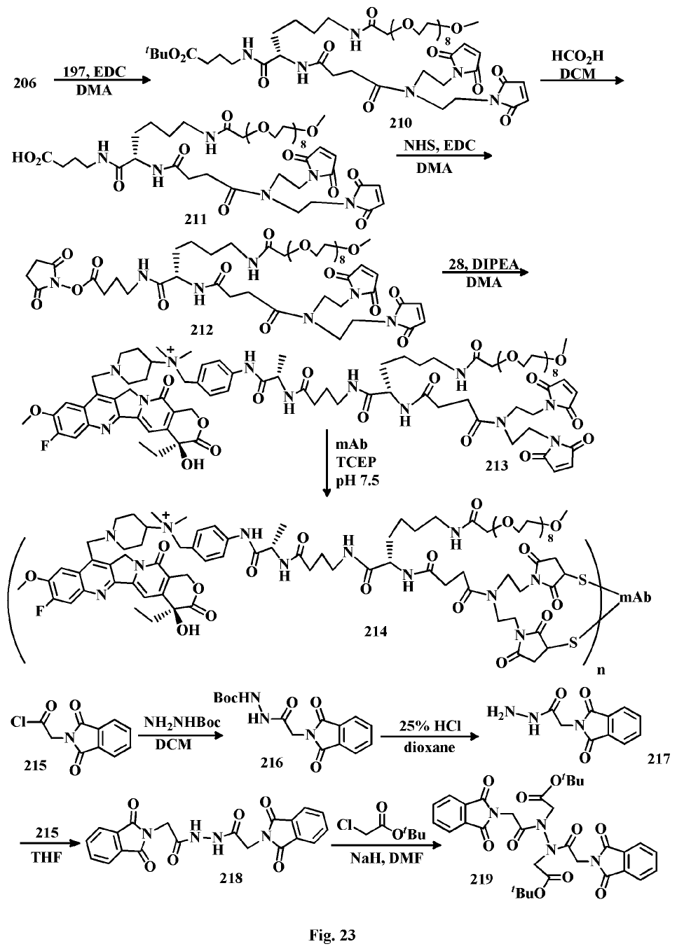

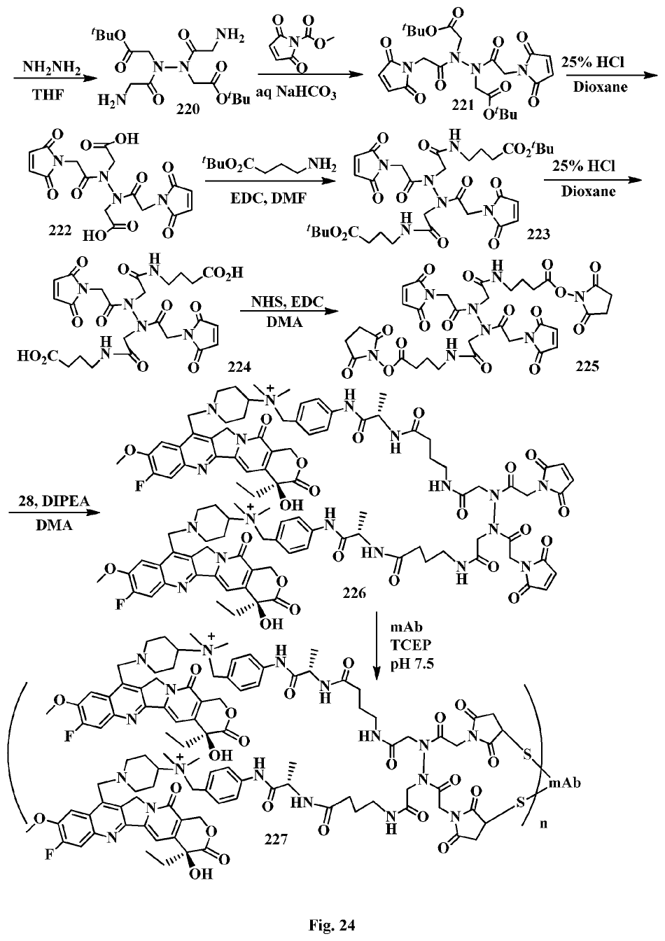

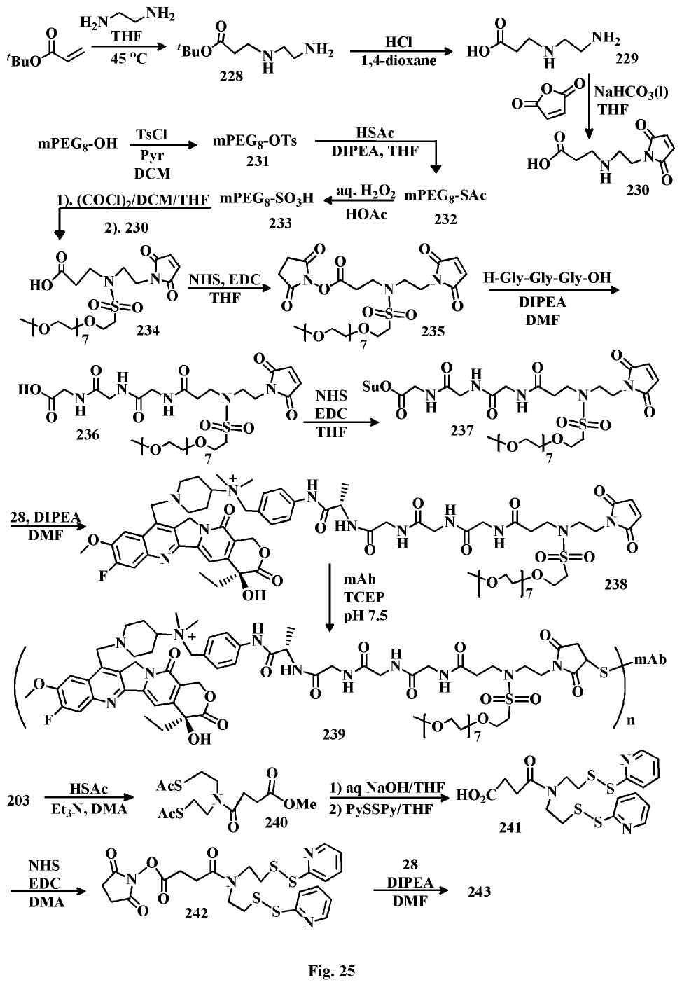

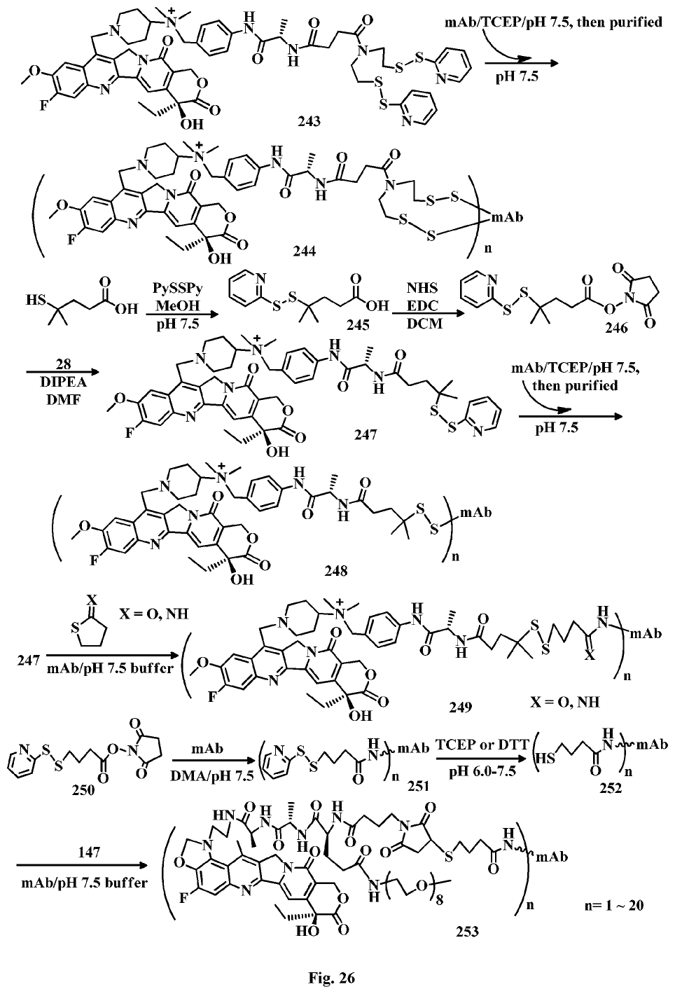

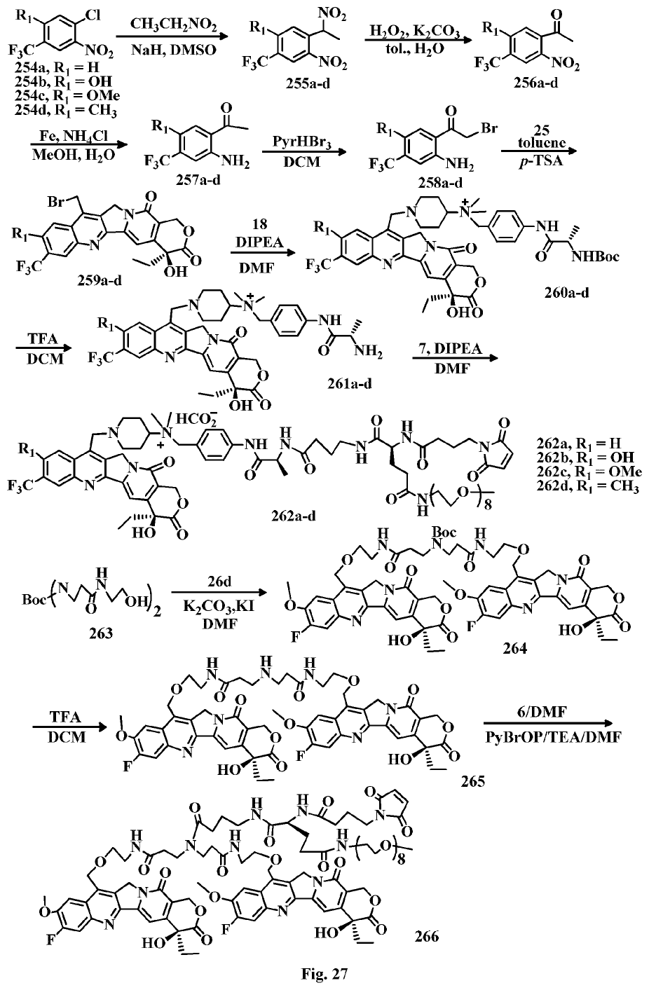

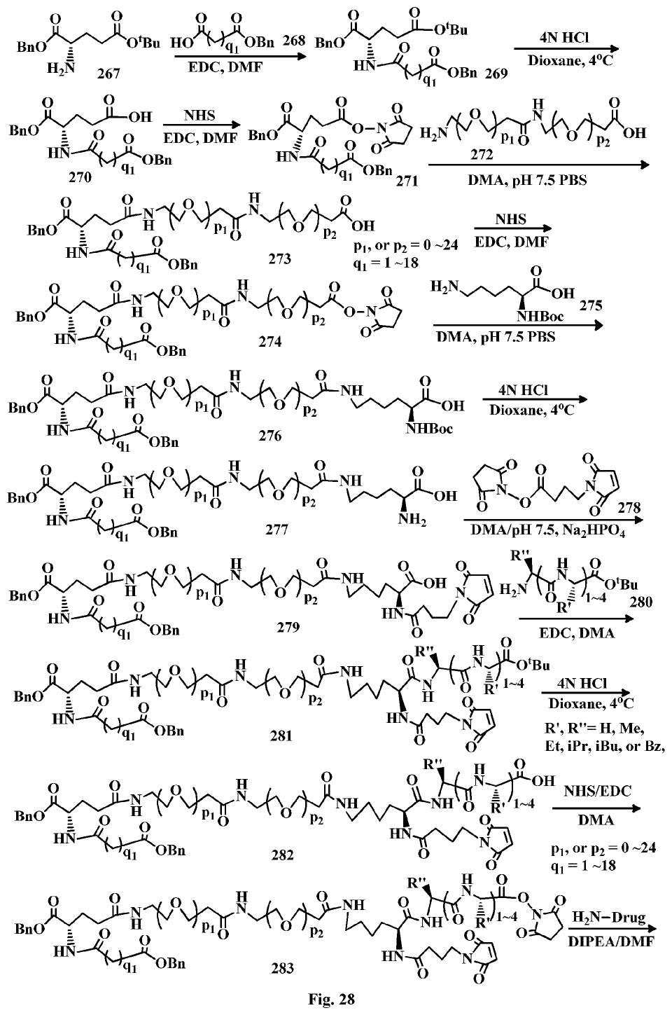

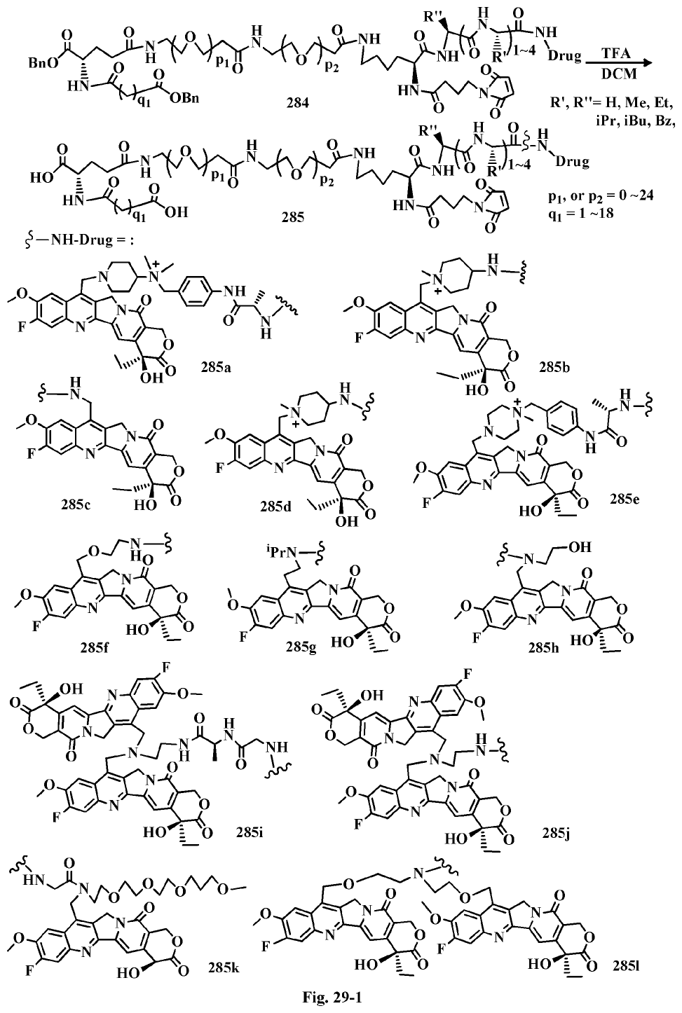

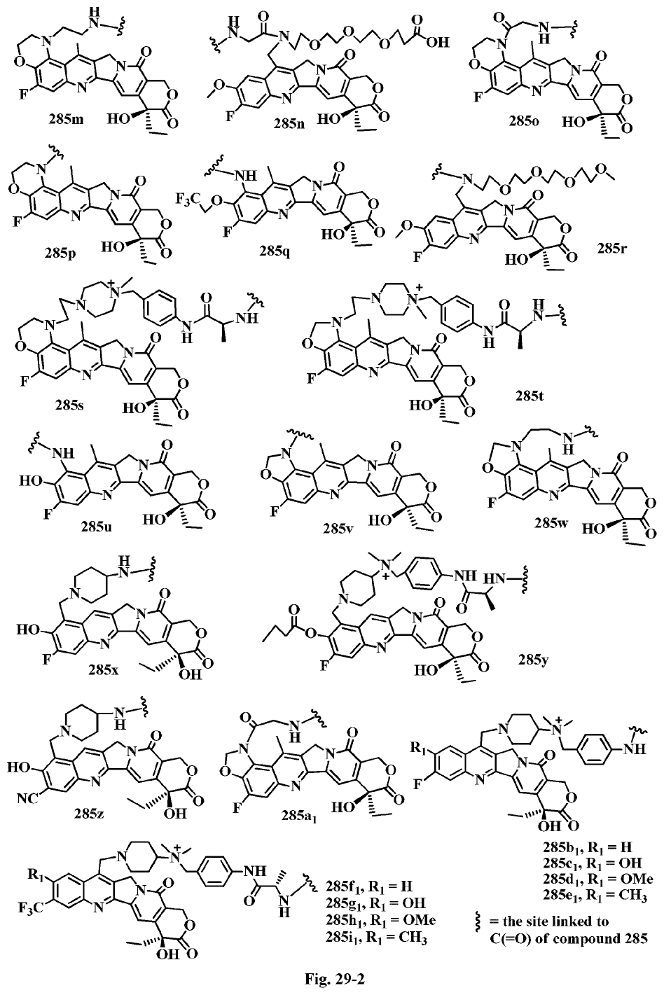

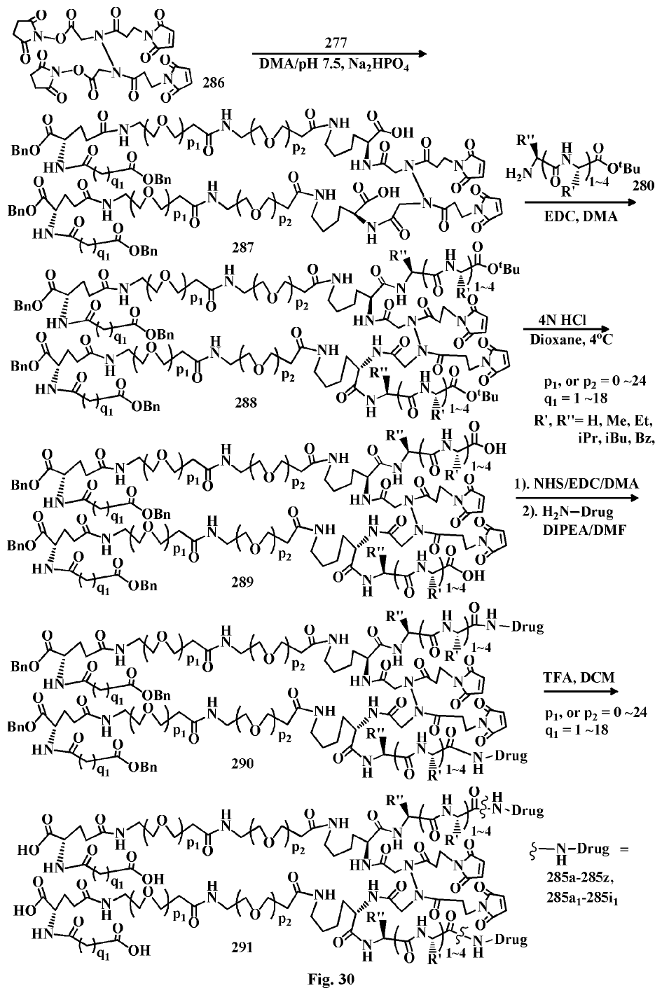

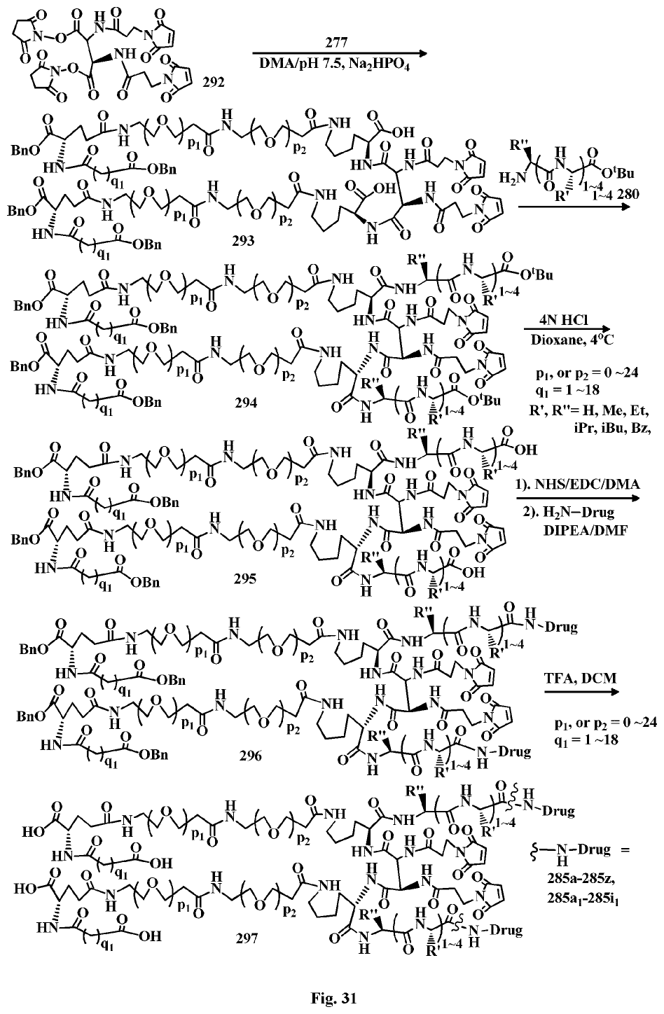

In another embodiment, the synthetic routes to produce the Camptothecin analogs and their conjugation to a cell-surface receptor binding molecules of the present invention are exampled, but not limited to, as shown in FIGS. 1-32.

In another embodiment, the releasable linker (L) is a chain of atoms selected from C, N, O, S, Si, and P that covalently connects the cell-surface binding ligand (T) to the potent Camptothecin analogs. The linker may have a wide variety of lengths, such as in the range from about 2 to about 100 atoms. The atoms used in forming the linker may be combined in all chemically relevant ways, such as forming alkylene, alkenylene, and alkynylene, ethers, polyoxyalkylene, esters, amines, imines, polyamines, hydrazines, hydrazones, amides, ureas, semicarbazides, carbazides, alkoxyamines, alkoxylamines, urethanes, amino acids, acyloxylamines, hydroxamic acids, and many others. In addition, it is to be understood that the atoms forming the releasable linker (L) may be either saturated or unsaturated, or may be radicals, or may be cyclized upon each other to form divalent cyclic structures, including cyclo alkanes, cyclic ethers, cyclic amines, arylenes, heteroarylenes, and the like in the linker.

The term releasable linker refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with a intracellular thiol, such as the amillimolar range of abundant of glutathione inside the malignant cells.

The releasable linker L of conjugates may have the formula: --Ww-(Aa)r-Vv-- wherein: --W-- is a Stretcher unit; w is 0 or 1; each --Aa-- is independently an Amino Acid unit; r is independently an integer ranging from 0 to 12; --V-- is a Spacer unit; and v is 0, 1 or 2.

The Stretcher unit (--W--), when present, links a targeted binding molecular unit (T) to an amino acid unit (--Aa--), or links to V when an Aa is not present. The Stretcher unit W may independently contain a self-immolative spacer, peptidyl units, a hydrazone bond, disulfides or thioether bond. In this regard a binding molecular (T) has a functional group that can form a bond with a functional group of a Stretcher. Useful functional groups that can be present on a binding molecular, either naturally or via chemical manipulation include, but are not limited to, sulfhydryl (—SH), amino, hydroxyl, carbonyl, the anomeric hydroxyl group of a carbohydrate, and carboxyl. Preferred functional groups are sulfhydryl, carboxy and amino. Sulfhydryl groups can be generated by reduction of an intramolecular disulfide bond of a Ligand. Alternatively, sulfhydryl groups can be generated by reaction of an amino group of a lysine moiety of a binding molecular using 2-iminothiolane (Traut's reagent) or thiolactone or another sulfhydryl generating reagent, such as modifies T with a disulfide bond linker, or a thiol ester following by reduction or hydrolysis respectively.

Illustrative examples of W linked to T have the structures:

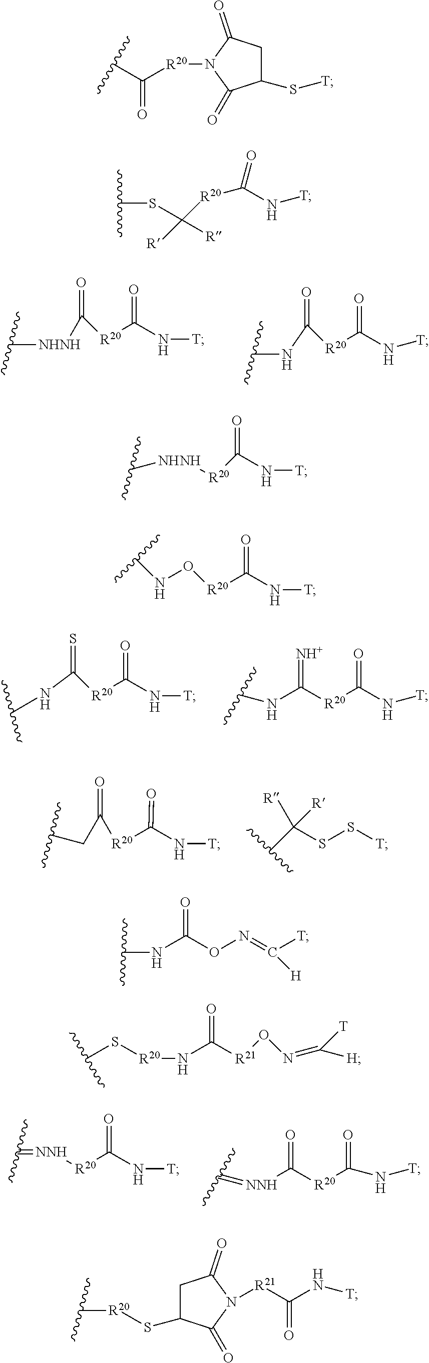

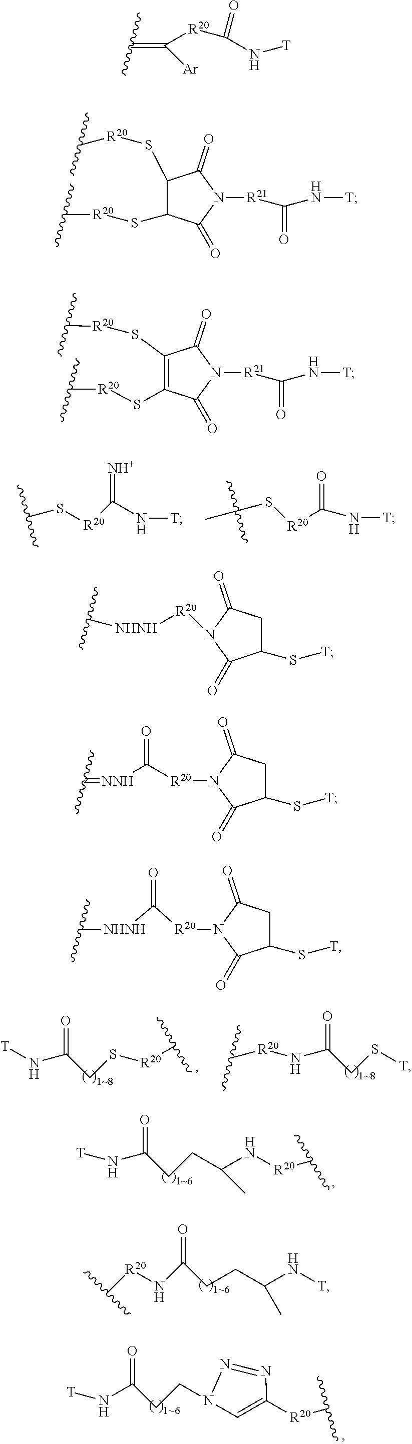

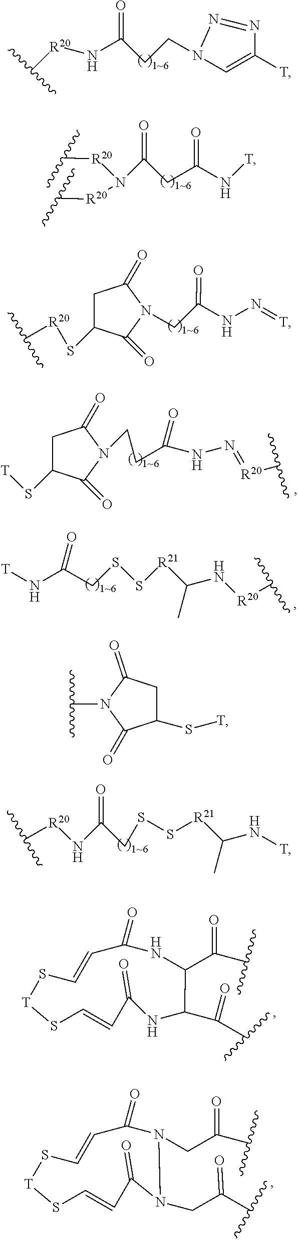

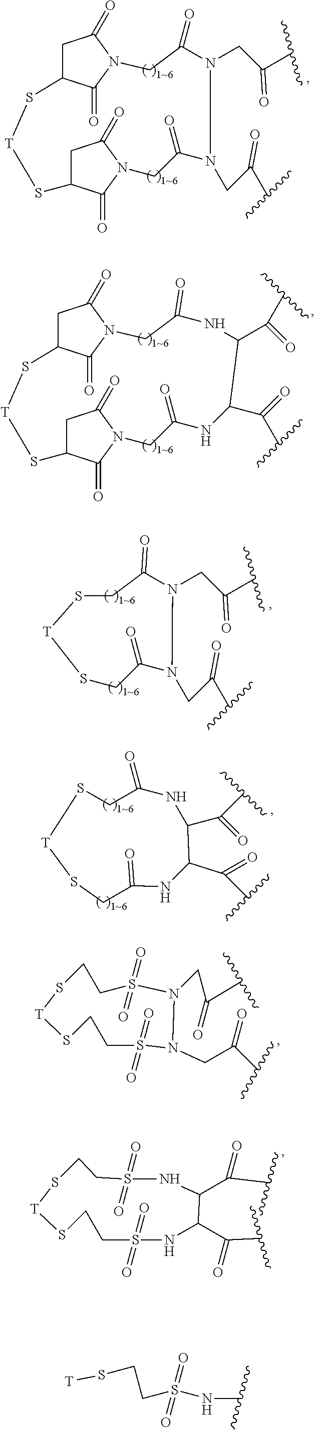

wherein R²⁰ and R²¹ are selected from —C₁˜C₉ alkylene-, —C₁˜C₇ carbocyclo-, —O—(C₁˜C₈ alkyl)-, -arylene-, —C₁˜C₉ alkylene-arylene-, -arylene, —C₁˜C₉ alkylene-, —C₁˜C₉ alkylene-(C₁˜C₈ carbocyclo)-, —(C₃˜C₇ carbocyclo)-C₁˜C₉ alkylene-, —C₃˜C₈ heterocyclo-, —C₁˜C₁₀ alkylene-(C₃˜C₈ heterocyclo)-, —(C₃˜C₈ heterocyclo)-C₁˜C₉ alkylene-, —(CH₂CH₂O)ₖ—, —(CH(CH₃)CH₂O)ₖ—, and —(CH₂CH₂O)ₖ—CH₂—; k is an integer ranging from 1-20; R′ and R″ are independently H or CH₃.

In another embodiment, conjugation of W to T covalently as illustrated above can be via various chemical reactions, which are typical conjugation methodologies:

Examples of the formation of amide linkages of the conjugates:

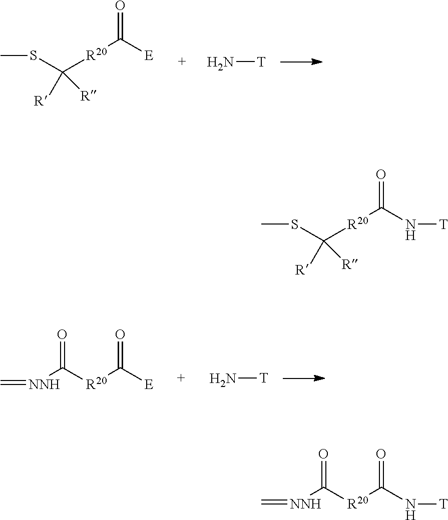

Wherein the Stretcher unit contains a reactive site of E, which can form an amide bond with a primary or secondary amino group of a Ligand. Example of the reactive E, includes, but is not limited to, such as hydroxysuccinimidyl esters (NHS, Sulfo-NHS, etc), 4-nitrophenyl esters, pentafluorophenyl esters, tetrafluorophenyl (includes sulfo-tetrafluorophenyl) esters, anhydrides, acid chlorides, sulfonyl chlorides, isocyanates and isothiocyanates.

Examples of thiol ether or disulfide bond linkages of the conjugates:

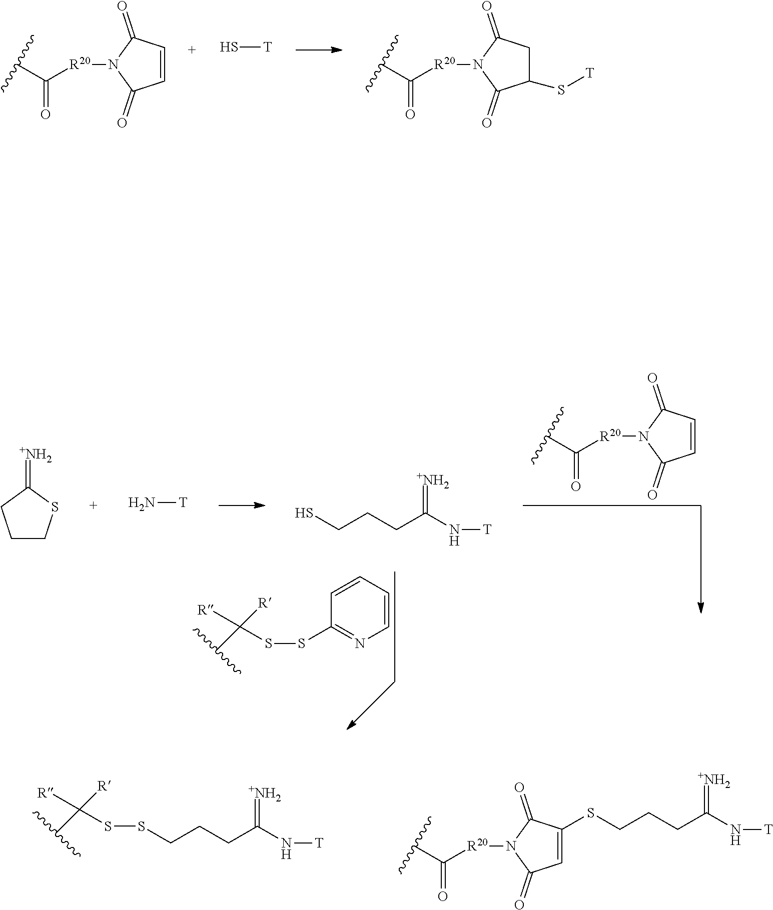

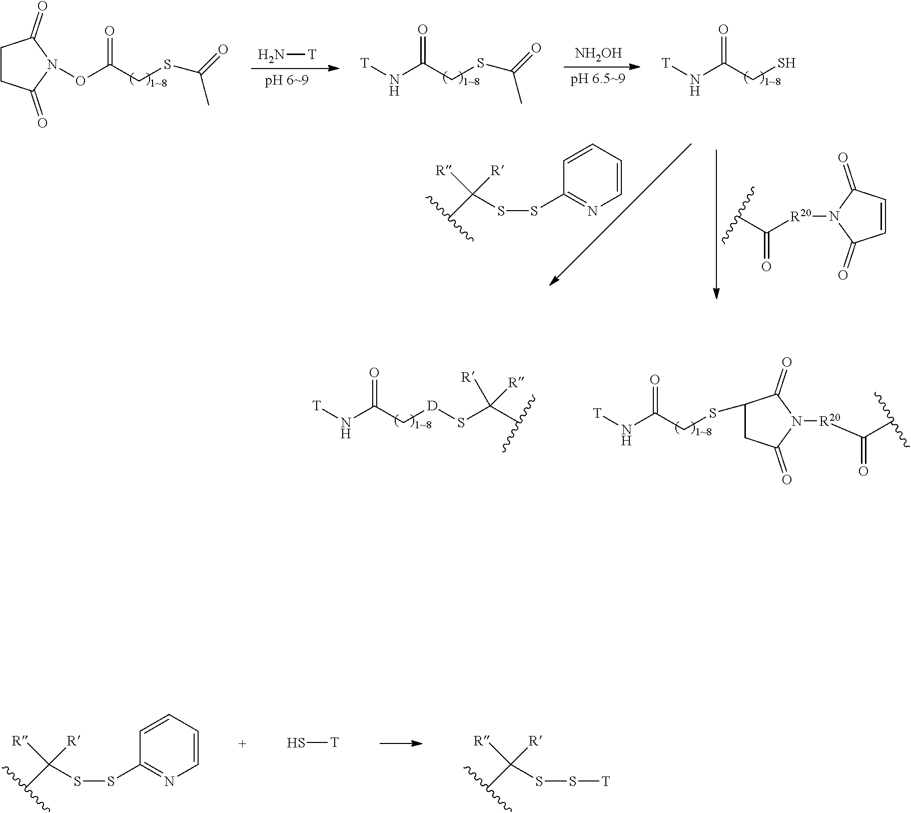

wherein the Stretcher unit contains a sulfhydryl reactive site, which can form a thiol ether or disulfide bond with a thiol group which is generated by reduction of an intramolecular disulfide bond of the binding ligand T, or generated by a chemical modification on the binding ligand T as shown in the above figure.

In yet another aspect of the invention, the reactive group of the Stretcher contains a reactive site that is reactive to an aldehyde (—CHO) or a ketone (—C(═O)R) group that can be chemically modified on a binding molecular T. For example, a carbohydrate on a binding molecular T can be mildly oxidized using a reagent such as sodium periodate to generate an aldehyde or a ketone (—C(═O)R) group; or an amine on an amino acid at the N-termini of antibodies (or proteins or peptides) can react with pyridoxal 5′-phosphate (PLP) in a buffer solution to introduce ketone groups (Scheck & Francis, ACS Chem. Biol. 2007, 2, 247-251). The resulting (—C═O) unit can be condensed with a Stretcher that contains a functionality such as a hydrazide, an oxime, a primary or secondary amine, a hydrazine, a thiosemicarbazone, a hydrazine carboxylate, and an arylhydrazide.

Examples of the conjugation of the hydrazone, or the oxime or imine linkages:

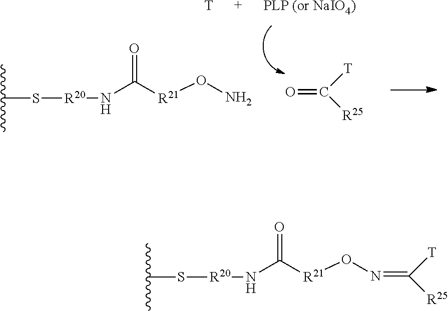

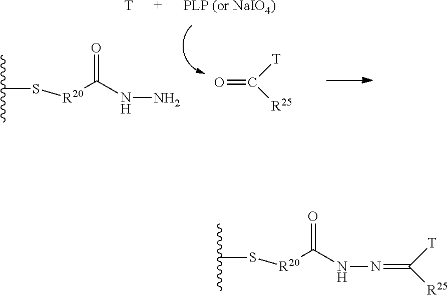

wherein R²⁰ and R²¹ are described above, R²⁵ is an organic substituent of an amino acid.

In another aspect of the invention, the Stretchers (which may contain a spacer V and/or an amino acid) can be linked to the binding molecules (T), followed by conjugation of a potent Camptothecin analog to the binding molecule-stretcher moiety in an aqueous buffered solution. Examples of these kinds of two-step conjugations (a drug linked to R¹⁶ is omitted here):

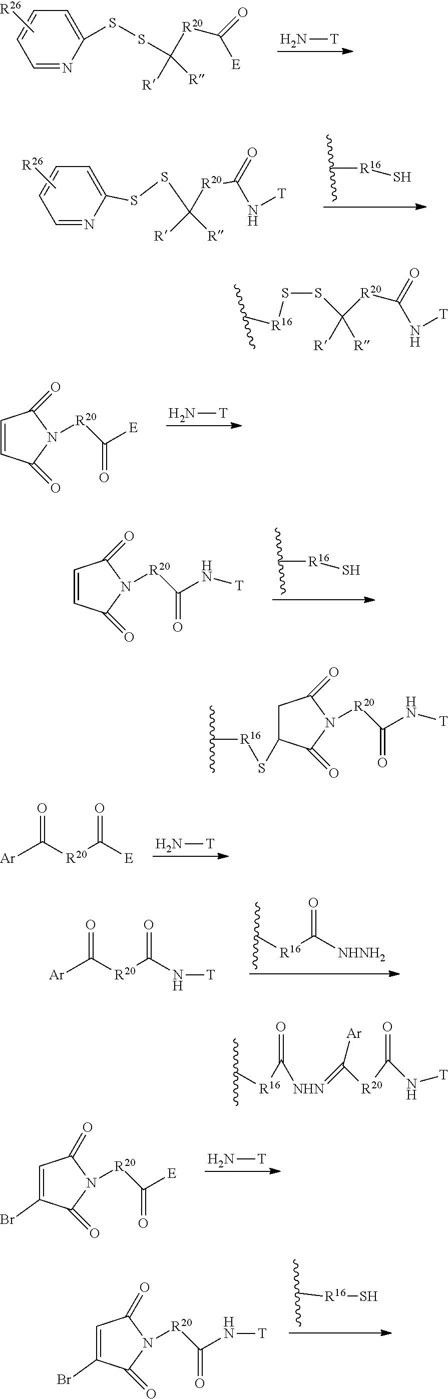

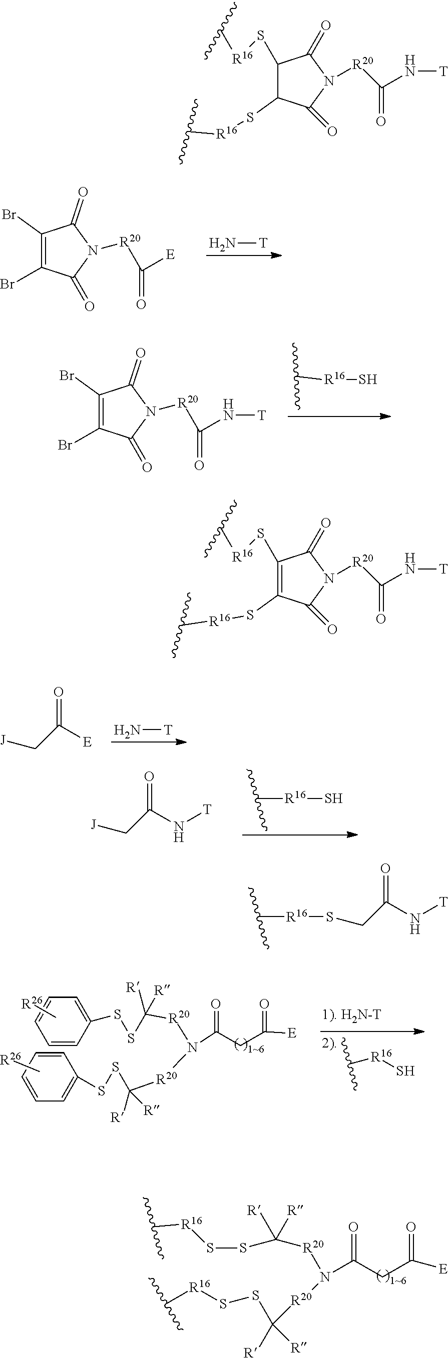

wherein E includes, but is not limited to, such as hydroxysuccinimidyl esters (NHS, Sulfo-NHS, etc), 4-nitrophenyl esters, pentafluorophenyl esters, tetrafluorophenyl (includes sulfo-tetrafluorophenyl) esters, anhydrides, acid chlorides, sulfonyl chlorides, isocyanates and isothiocyanates. R′ and R″ are independently H or CH₃; R²⁰, R¹⁶ and Ar are defined in various embodiment throughout this inventions; R²⁶ is H, or F, or NO₂ independently; J is F, Cl, Br, I, tosylate (TsO) or mesylate (MsO) independently and wherein

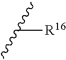

bears at least one Camptothecin analog/drug as shown

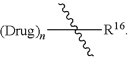

In another aspect of the invention, the Stretchers can be linked to a potent Camptothecin analog first, followed by conjugation of the binding molecules (T) in an aqueous pH 3˜10 (preferably pH 5˜8.5) buffered solution containing up to 50% of organic cosolvents. Examples of these kinds of two-step conjugations:

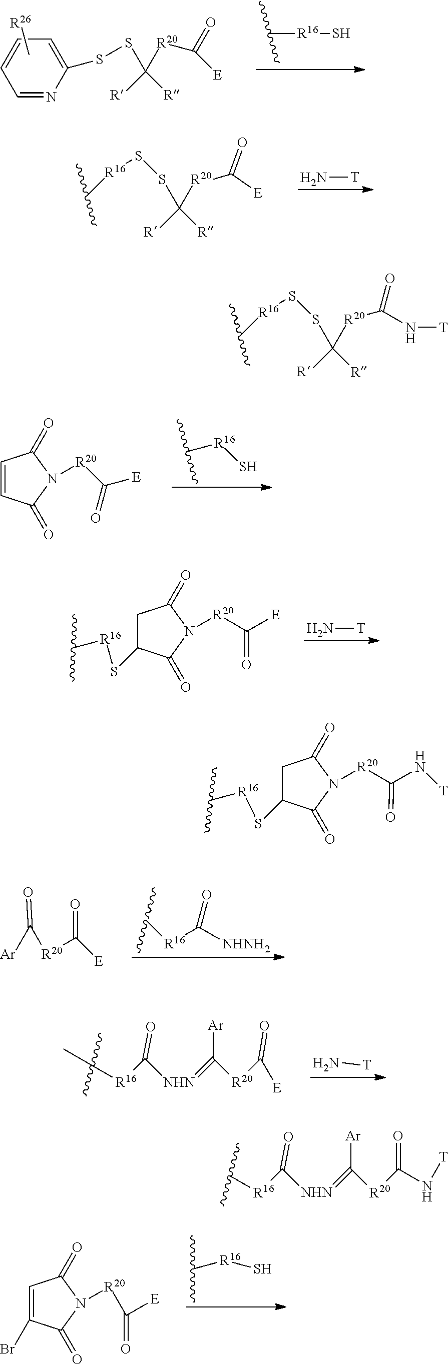

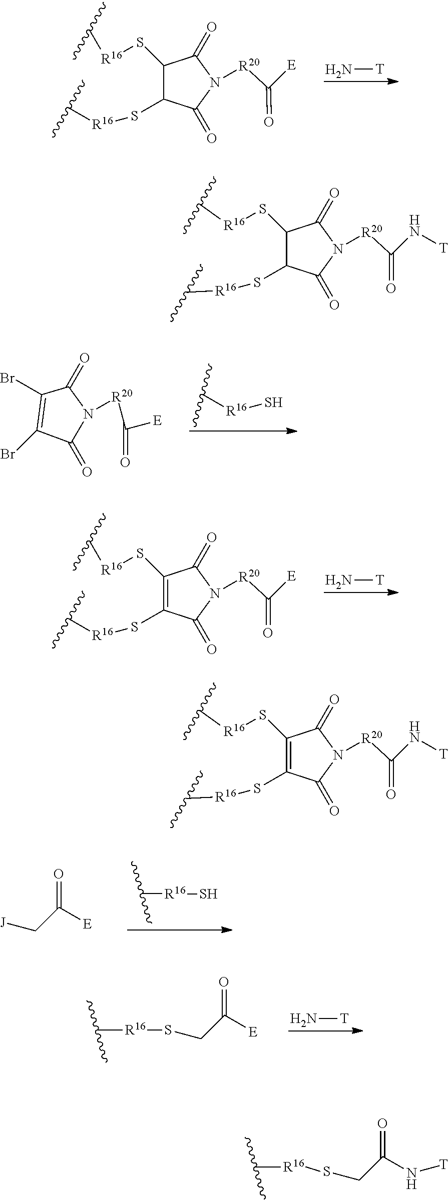

wherein E includes, but is not limited to, such as hydroxysuccinimidyl esters (NHS, Sulfo-NHS, etc), 4-nitrophenyl esters, pentafluorophenyl esters, tetrafluorophenyl (includes sulfo-tetrafluorophenyl) esters, anhydrides, acid chlorides, sulfonyl chlorides, isocyanates and isothiocyanates. R′ and R″ are independently H or CH₃; R¹⁶, R²⁰ and Ar are defined in various embodiment throughout this inventions; R²⁶ is H, or F, or NO₂ independently; J is F, Cl, Br, I, tosylate (TsO) or mesylate (MsO) independently and wherein

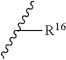

bears at least one Camptothecin analog/drug.

The Amino Acid unit (-Aa-), when present, links the Stretcher unit to the Spacer unit if the Spacer unit is present, links the Stretcher unit to the Camptothecin analog unit if the Spacer unit is absent, and links the binding molecule (T) unit to the Camptothecin analog unit if the Stretcher unit and Spacer unit are absent. -(Aa)r- is a natural or unnatural amino acid, the same or different sequences of amino acids of dipeptide, tripeptide, tetrapeptide, pentapeptide, hexapeptide, heptapeptide, octapeptide, nonapeptide, decapeptide, undecapeptide or dodecapeptide unit, and r is an integer ranging from 0 to 12. The term amino acid as used herein refers generally to aminoalkylcarboxylate, where the alkyl radical is optionally substituted, such as with alkyl, acyl, hydroxy alkyl, sulfhydrylalkyl, aminoalkyl, carboxyalkyl, and the like, The structures of the natural and unnatural amino acids and peptides are described in the book: G. C. Barrett and D. T. Elmore, “Amino Acid and Peptide”, Cambridge University Press, 2004. In addition, amino acid refers to beta, gamma, and longer amino acids with intra chain containing methyl, benzyl, hydroxymethyl, thiomethyl, carboxyl, carboxylmethyl, guanidinopropyl, and the like. More preferably the amino acid is selected from asparagine, aspartic acid, cysteine, glycine, glutamic acid, lysine, glutamine, arginine, serine, ornithine, threonine, and the like.

The Amino Acid unit of the invention can be enzymatically cleaved by one or more enzymes, including a tumor-associated protease, to liberate the Camptothecin analog, which in one embodiment is protonated in vivo upon release to provide a Camptothecin analog.

The Spacer unit (-V-), when present, links an Amino Acid unit to the Camptothecin analog when an Amino Acid unit is present. Alternately, the Spacer unit links the Stretcher unit to Camptothecin analog when the Amino Acid unit is absent. The Spacer unit also links to Camptothecin analog and to the binding molecule (T) when both the Amino Acid unit and Stretcher unit are absent. The spacer linkers may contain function groups that substantially increase the water solubility, biological transport, preferential renal clearance, uptake, absorption, biodistribution, and/or bioavailability of the conjugate are described herein. Spacer units are of two general types: self-immolative and non-self-immolative. A non-self-immolative Spacer unit is one in which part or all of the Spacer unit remains bound to Camptothecin analog after cleavage, particularly enzymatic, of an Amino Acid unit from the Camptothecin analog-Linker-binding molecule conjugate or the Camptothecin analog-Linker Compound. The self-immolative unit includes aromatic compounds that are electronically similar to para-aminobenzyl-carbamoyl (PAB) groups, 2-aminoimidazol-5-methanol derivatives, heterocyclic PAB analogs, beta-glucuronide, and ortho or para-aminobenzylacetals; or one of the following structures:

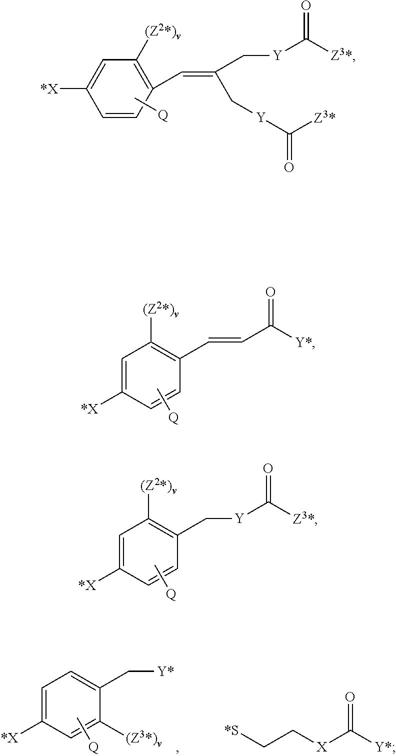

wherein the (*) atom is the point of attachment of additional spacer or releasable linker units, the amino acids (Aa)ᵣ, the camptothecin analog, and/or the binding molecule (T); X, Y and Z are independently NH, O, or S; Z² is H, NH, O or S independently. v is 0 or 1; Q is independently H, OH, C₁-C₆ alkyl, (OCH₂CH₂)ₙ, F, Cl, Br, I, OR¹⁷, or SR¹⁷, NR¹⁷R″, N═NR¹⁷, N═R¹⁷, NR¹⁷R¹⁸, NO₂, SOR¹⁷R¹⁸, SO₂R¹⁷, SO₃R¹⁷, OSO₃R¹⁷, PR¹⁷R¹⁸, POR¹⁷R¹⁸, PO₂R¹⁷R¹⁸, OPO(OR¹⁷)(OR¹⁸), or OCH₂PO(OR¹⁷)(OR¹⁸), wherein R¹⁷, R¹⁸ are independently H, C₁˜C₈ of alkyl; C₂˜C₈ of alkenyl, alkynyl, heteroalkyl; C₃˜C₈ of aryl, heterocyclic, carbocyclic, cycloalkyl, heterocycloalkyl, heteroaralkyl, alkylcarbonyl; or pharmaceutical cation salts; v is an integer ranging from 1-20;

Examples of the non-self-immolative spacer linker units (-V-):

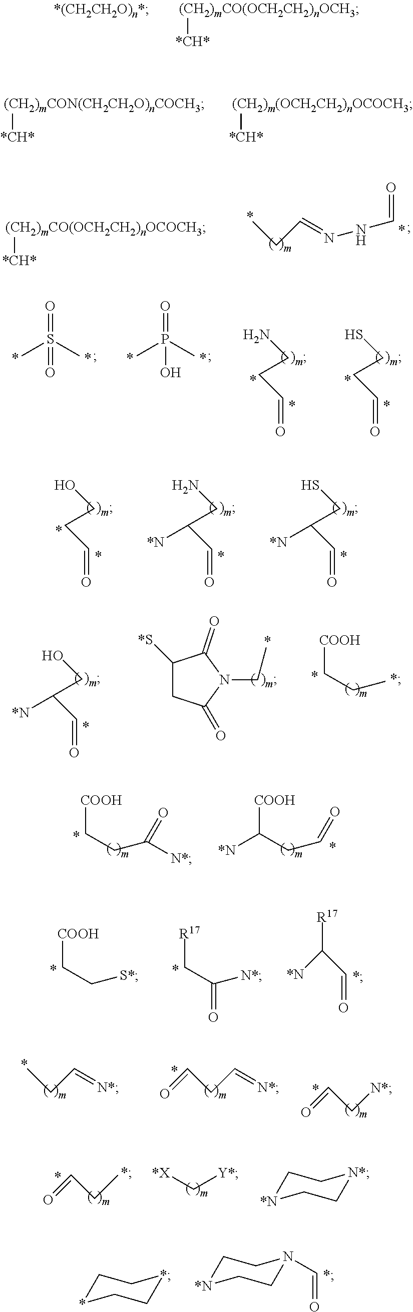

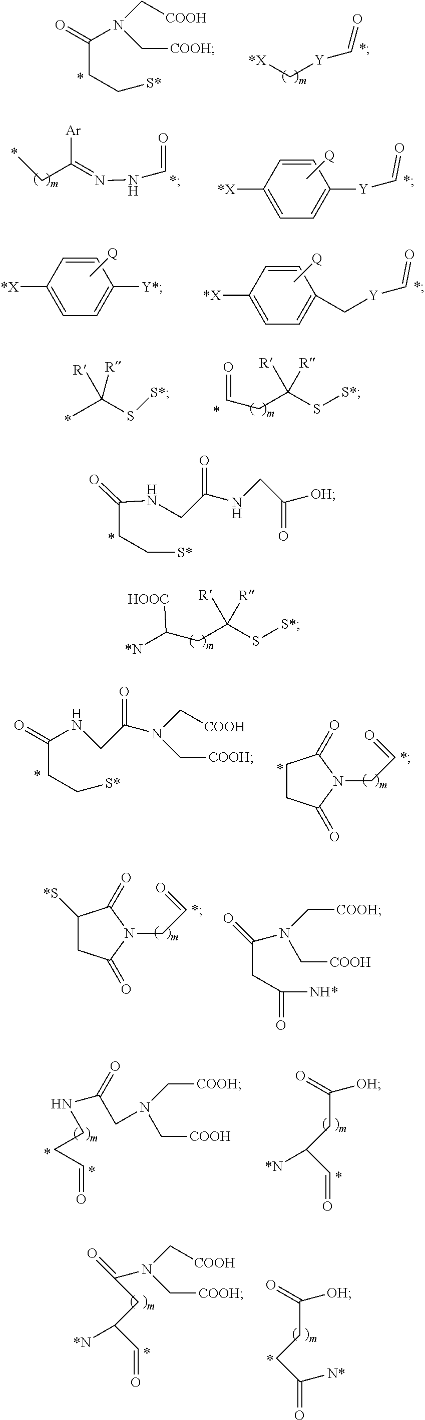

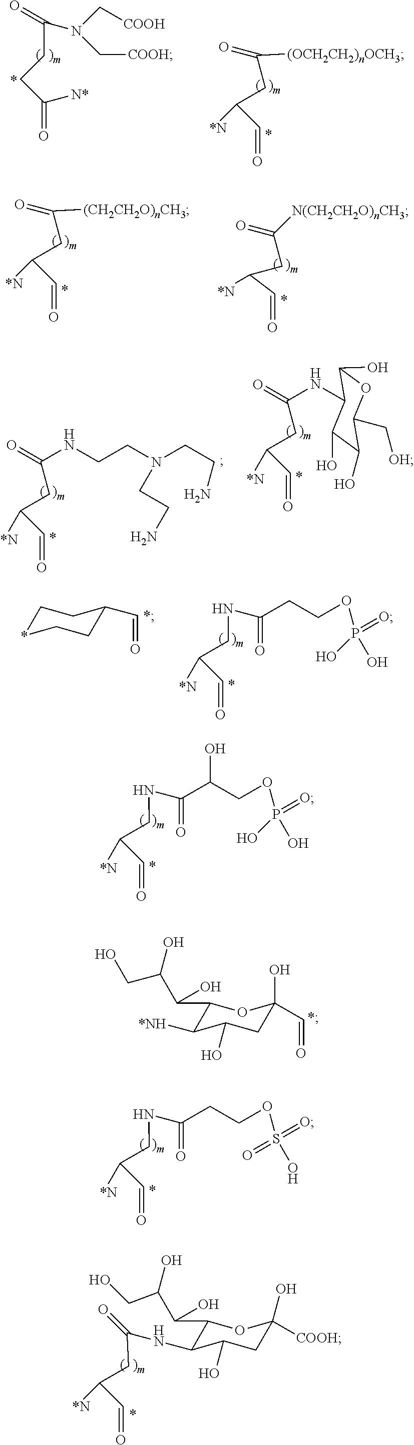

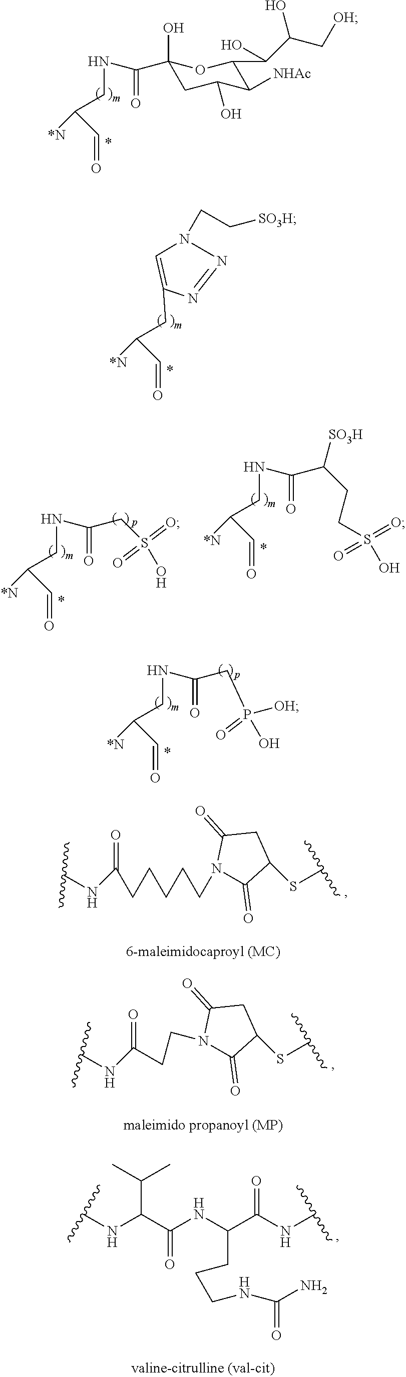

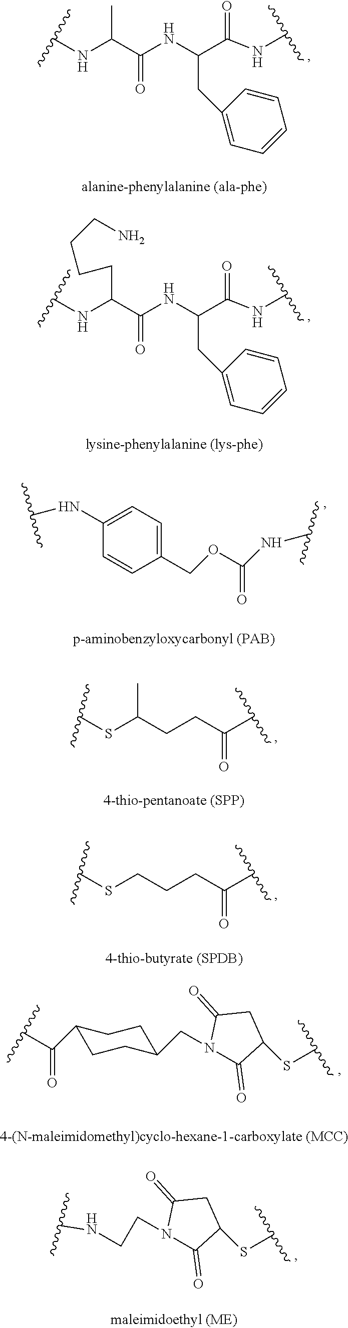

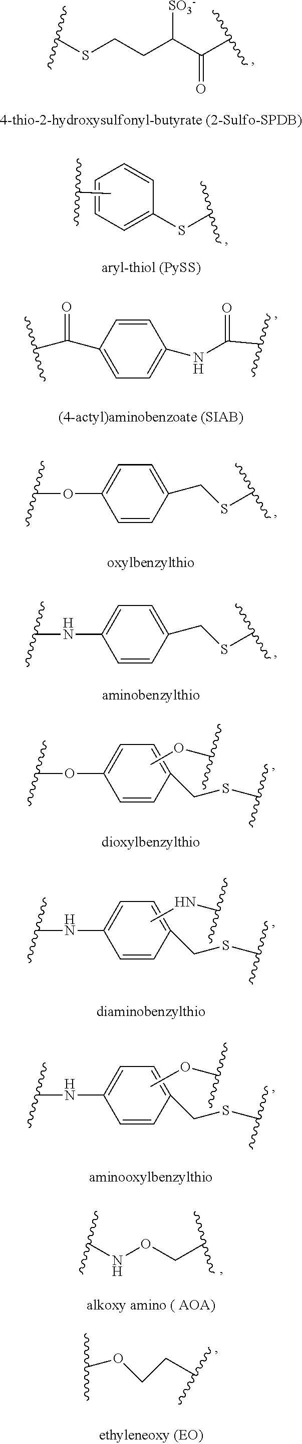

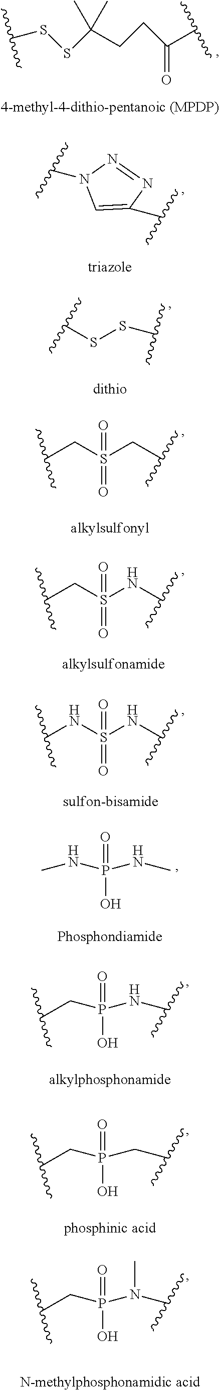

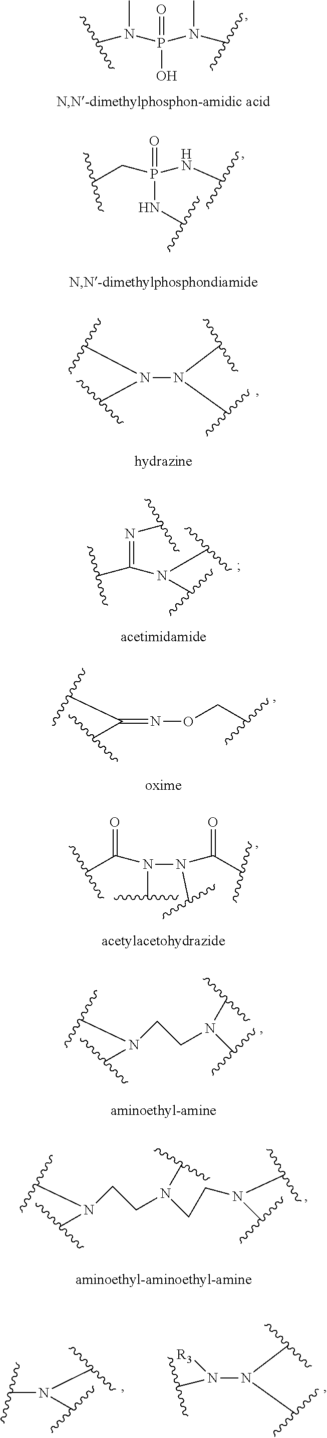

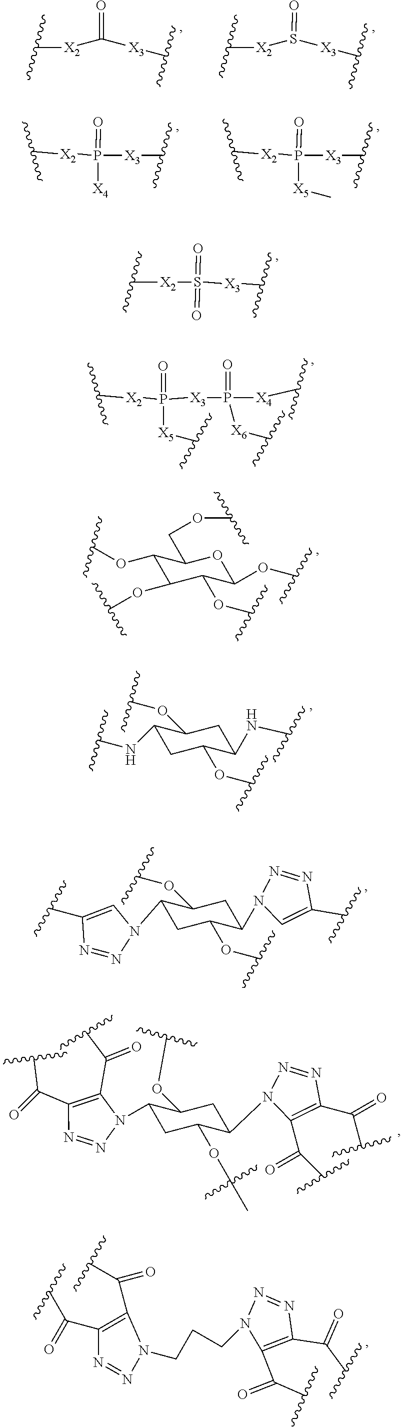

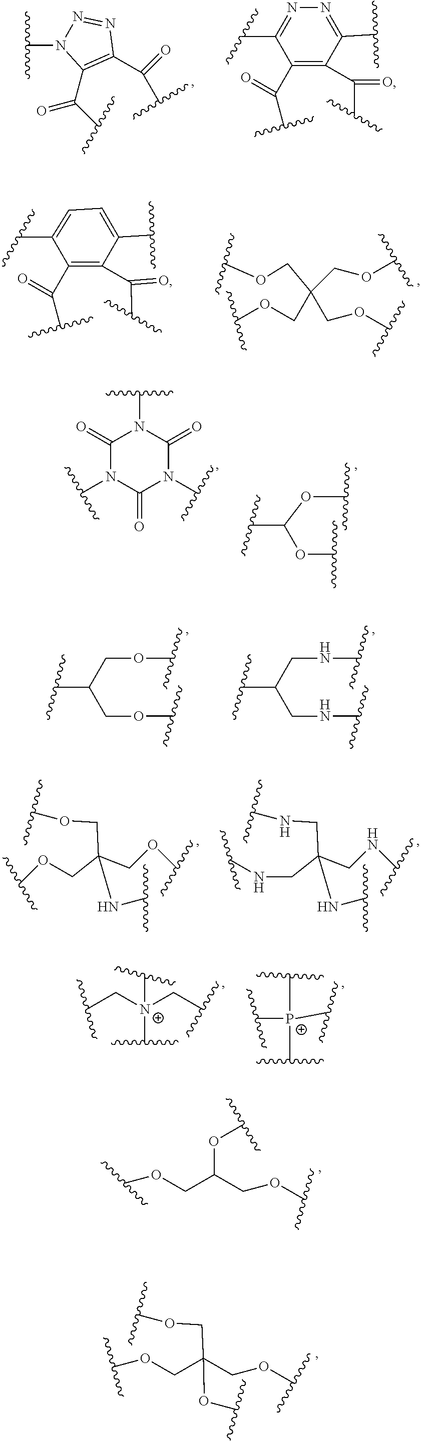

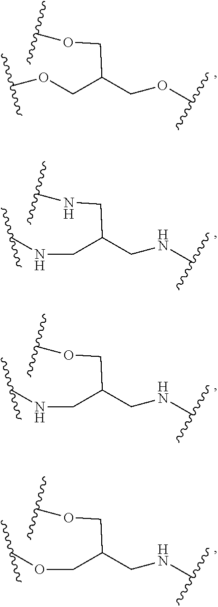 [SMILES: *OCC(CO*)CO* ; *NCC(CN*)CN* ; *NCC(CN*)CO* ; *NCC(CO*)CO*]

or L- or D-, natural or unnatural peptides containing 1-20 the same or different amino acids;

Wherein “*” and “ [symbol] ” atom are the point of attachment of additional spacer or releasable linkers, the Camptothecin analogs, and/or the binding molecules; m is 1-10; n is 1-20; X₂, X₃, X₄, X₅, or X₆, are independently selected from NH; NHNH; N(R₁₂); N(R₁₂)N(R_{12′}); O; S; C₁-C₆ of alkyl; C₂-C₆ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids; wherein R₁₂ and R_{12′} are independently H; C₁-C₈ of alkyl; C₂-C₈ of hetero-alkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or polyethyleneoxy unit of formula (OCH₂CH₂)ₚ or (OCH₂CH(CH₃))ₚ, wherein p is an integer from 0 to about 1000, or combination above thereof.

A releasable component of the linker L that at least one bond in L can be broken under physiological conditions: a pH-labile, acid-labile, base-labile, oxidatively labile, metabolically labile, biochemically labile or enzyme-labile bond, which having one of the following structures: —(CR₁₅R₁₆)ₘ(Aa)r(CR₁₇R₁₈)ₙ(OCH₂CH₂)ₜ—, —(CR₁₅R₁₆)ₘ(CR₁₇R₁₈)ₙ(Aa)ₜ(OCH₂CH₂)ₜ—, - (Aa)ᵣ-(CR₁₅R₁₆)ₘ(CR₁₇R₁₈)ₙ(OCH₂CH₂)ₜ—, —(CR₁₅R₁₆)ₘ(CR₁₇R₁₈)ₙ(OCH₂CH₂)ᵣ(Aa)ₜ-, —(CR₁₅R₁₆)ₘ—(CR₁₇═CR₁₈)(CR₁₉R₂₀)ₙ(Aa)ₜ(OCH₂CH₂)ᵣ—, —(CR₁₅R₁₆)ₘ(NR₁₁CO)(Aa)ₜ(CR₁₉R₂₀)ₙ—(OCH₂CH₂)ᵣ—, —(CR₁₅R₁₆)ₘ(Aa)ₜ(NR₂₁CO)(CR₁₉R₂₀)ₙ(OCH₂CH₂)ᵣ—, —(CR₁₅R₁₆)ₘ(OCO)(Aa)ₜ(CR₁₉R₂₀)ₙ(OCH₂—CH₂)ᵣ—, —(CR₁₅R₁₆)ₘ(OCNR₁₇)(Aa)ₜ(CR₁₉R₂₀)ₙ(OCH₂CH₂)ᵣ—, —(CR₁₅R₁₆)ₘ—(CO)(Aa)ₜ(CR₁₉R₂₀)ₙ—(OCH₂CH₂)ᵣ—, —(CR₁₅R₁₆)ₘ(NR₂₁CO)(Aa)ₜ(CR₁₉R₂₀)ₙ(OCH₂CH₂)ᵣ—, —(CR₁₅R₁₆)ₘ(OCO)(Aa)ₜ-(CR₁₉R²⁰)ₙ(OCH₂CH₂)ᵣ—, —(CR₁₅R₁₆)ₘ(OCNR₁₇)(Aa)ₜ(CR₁₉R₂₀)ₙ—(OCH₂CH₂)ᵣ—, —(CR₁₅R₁₆)ₘ—(CO)(Aa)ₜ(CR₁₉R²⁰)ₙ—(OCH₂CH₂)ᵣ—, —(CR₁₅R₁₆)ₘ-phenyl-CO(Aa)ₜ-(CR₁₇R₁₈)ₙ—, —(CR₁₅R₁₆)ₘ-furyl-CO(Aa)ₜ(CR₁₇R₁₈)ₙ—, —(CR₁₅R⁶)ₘ-oxazolyl-CO(Aa)ₜ(CR₁₇R₁₈)ₙ—, —(CR₁₅R₁₆)ₘthiazolylCO-(Aa)ₜ(CCR₁₇R₁₈)ₙ—, —(CR₁₅R₁₆)ₜ-thienyl-CO(CR₁₇R₁₈)ₙ—, —(CR₁₅R₁₆)ₜ-imidazolyl-CO—(CR₁₇R₁₈)ₙ—, —(CR₁₅R₁₆)ₜ-morpholino-CO(Aa)ₜ(CR₁₇R₁₈)ₙ—, —(CR₁₅R₁₆)ₜ-piperazino-CO(Aa)ₜ(CR₁₇R₁₈)ₙ—, —(CR₁₅R₁₆)ₜN-methylpiperazin-CO(Aa)ₜ(CR₁₇R₁₈)ₙ—, —(CR₁₅R₁₆)ₘ-(Aa)ₜphenyl-, —(CR₁₅R₁₆)ₘ-(Aa)ₜfuryl-, —(CR₁₅R₁₆)ₘ-oxazolyl(Aa)ₜ-, —(CR₁₅R₁₆)ₘ-thiazolyl(Aa)ₜ-, —(CR₁₅R₁₆)ₘ-thienyl-(Aa)ₜ-, —(CR₁₅R₁₆)ₘ-imidazolyl(Aa)ₜ-, —(CR₁₅R₁₆)ₘ₋morpholino-(Aa)ₜ-, —(CR₁₅R₁₆)ₘ-piperazino-(Aa)ₜ-, —(CR₁₅R₁₆)ₘ—N-methylpiperazino-(Aa)ₜ-, —K(CR₁₅R₁₆)ₘ(Aa)ᵣ(CR₁₇R₁₈)ₙ(OCH₂CH₂)ₜ—, —K—(CR₁₅R₁₆)ₘ(CR₁₇R₁₈)ₙ(Aa)ᵣ(OCH₂CH₂)ₜ—, —K(Aa)ᵣ-(CR₁₅R₁₆)ₘ(CR₁₇R₁₈)ₙ(OCH₂CH₂)ₜ—, —K—(CR₁₅R₁₆)ₘ(CR₁₇R₁₈)ₙ(OCH₂CH₂)ᵣ(Aa)ₜ-, —K(CR₁₅R₁₆)ₘ(CR₁₇═CR₁₈)(CR₁₉R₂₀)ₙ(Aa)ₜ(OCH₂—CH₂)ᵣ, —K(CR₁₅R₁₆)ₘ(NR₁₁CO)(Aa)ₜ-(CR₁₉R₂₀)ₙ(OCH₂CH₂)ᵣ—, —K(CR₅R₆)ₘ(Aa)ₜ(NR₂₁CO)—(CR₁₉R₂₀)ₙ(OCH₂CH₂)ᵣ—, —K(CR₁₅R₁₆)ₘ—(OCO)(Aa)ₜ(CR₁₉R₂₀)ₙ—(OCH₂CH₂)ᵣ—, —K(CR₁₅R₁₆)ₘ (O—CNR₁₇)(Aa)ₜ(CR₁₉R₂₀)ₙ—(OCH₂CH₂)ᵣ—, —K(CR₁₅R₁₆)ₘ(CO)(Aa)ₜ-(CR₁₉R₂₀)ₙ(OCH₂CH₂)ᵣ—, —K(CR₁₅R₁₆)ₘ(NR₂₁CO)(Aa)ₜ(CR₁₉R₂₀)ₙ(OCH₂CH₂)ᵣ—, —K(CR₁₅R₁₆)ₘ—(OCO)(Aa)ₜ(CR₁₉R₂₀)ₙ(O—CH₂CH₂)ᵣ—, —K(CR₁₅R₁₆)ₘ(OCNR₁₇)(Aa)ₜ(CR₁₉R₂₀)ₙ(OCH₂CH₂)ᵣ—, —K—(CR₁₅R₁₆)ₘ(CO)(Aa)ₜ-(CR₁₉R₂₀)ₙ(OCH₂CH₂)ᵣ—, —K(CR₁₅R¹⁶)ₘ-phenyl-CO(Aa)ₜ(CR₁₇R₁₈)ₙ—, —K—(CR₁₅R¹⁶)ₘ-furyl-CO-(Aa)ₜ(CR₁₇R₁₈)ₙ—, —K(CR₁₅R₁₆)ₘ-oxazolyl-CO(Aa)ₜ(CR₁₇R₁₈)ₙ—, —K(CR₁₅R₁₆)ₘ-thiazolyl-CO(Aa)ₜ-(CR₁₇R₁₈)ₙ—, —K(CR₁₅R₁₆)ₜ-thienyl-CO(CR₁₇R₁₈)ₙ—, —K(CR₁₅R₁₆)ₜimidazolyl-CO—(CR₁₇R₁₈)ₙ—, —K—(CR₅R₆)ₜmorpholino-CO(Aa)ₜ-(CR₁₇R₁₈)ₙ—, —K(CR₁₅R₁₆)ₜ-piperazino-CO(Aa)ₜ(CR₁₇R₁₈)ₙ—, —K—(CR₁₅R₁₆)ₜ—N-methylpiperazin-CO(Aa)ₜ(CR₁₇R₁₈)ₙ—, —K(CR₁₅R₁₆)ₘ(Aa)ₜphenyl, —K(CR₁₅R₁₆)ₘ-(Aa)ₜfuryl-, —K(CR₁₅R₁₆)ₘ-oxazolyl-(Aa)ₜ-, —K(CR₁₅R₁₆)ₘ-thiazolyl(Aa)ₜ-, —K(CR₁₅R₁₆)ₘ-thienyl-(Aa)ₜ—, —K(CR₁₅R₁₆)ₘ-imidazolyl(Aa)ₜ-, —K(CR₁₅R₁₆)ₘ-morpholino(Aa)ₜ-, —K(CR₁₅R₁₆)ₘpiperazino(Aa)ₜ, —K(CR₁₅R₁₆)ₘ—N-methyl-piperazino(Aa)ₜ-; wherein Aa, m, n, are described above; t and r here are 0-100 independently; R₁₃, R₁₄, R₁₅, R₁₆, R₁₇, R₁₈, R₁₉, R₂₀, and R₂₁ are independently chosen from H; halide; C₁˜C₈ of alkyl or heteroalkkyl, C₂˜C₈ of aryl, alkenyl, alkynyl, ether, ester, amine or amide, C₃˜C₈ of aryl, which optionally substituted by one or more halide, CN, NR₁₂R_{12′}, CF₃, OR₁₂, Aryl, heterocycle, S(O)R₁₂, SO₂R₁₂, —CO₂H, —SO₃H, —OR₁₂, —CO₂R₁₂, —CONR₁₂, —PO₂R₁₂R₁₃, —PO₃H or P(O)R₁₂R₁₂·R₁₃; K is NH, NR₁₂, —SS—, —C(═O)—, —C(═O)NH—, —C(═O)O—, —C═NH—O—, —C═N—NH—, —C(═O)NH—NH, O, S, Se, B, Het (heterocyclic or hetero-aromatic ring having C₃-C₁₂); or peptides containing the same or different 1-20 amino acids.

In yet another aspect of the invention, the linker L is preferably containing an amino, sulfonamide, phosphamide or amino acid group wherein the formula (I-q) can be linked to the Linker L as a side chain. The amino acids in the linker L are preferably selected from an aspartic acid, a glutamic acid, a lysine, an ornithine, or a tyrosine wherein one or two of their functional amino group, carboxylic group or phenol group can link the long side chain of the formula (I-q):

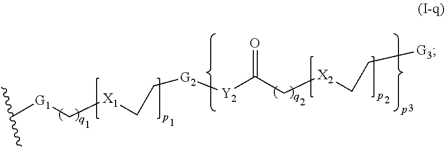

wherein [symbol] is the site linked to the sulfonyl, phosphate, amino, or carbonyl group in the linker, preferably the carbonyl, amino or phenol group of the aspartic acid, glutamic acid, lysine ornithine or tyrosine in the linker accordingly; G₁ is NH, NHNH, C(═O), NHNHC(O), C(═O)NH, C(═NH)NH, CH₂, CH₂C(O), C(O)O, NHC(O)NH, or (Aa)ᵣ, (r=1-12); G₂ is NH, NHNH, C(═O), NHNHC(O), C(═O)NH, C(═NH)NH, CH₂, C(O)O, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, (Aa)ᵣ, (r=1-12); X₁ and X₂ are independently O, CH₂, S, NH, N(R₁₂), ⁺NH(R₁₂), ⁺N(R₁₂)(R₁₃), C(O), OC(O), OC(O)O, NHSO₂NH, NHP(O)(NH)₂, SO₂NH, P(O)(NH)₂, NHS(O)NH, NHP(O)(OH)(NH), OC(O)NH, NHC(O)NH; Y₂ is O. NH, NR₁, CH₂. S. Ar; G₃ is OH, SH, OR₁, SR₁, OC(O)R₁, NHC(O)R₁₂, C(O)R₁₂, CH₃, NH₂, NR₁₂, ⁺NH(R₁₂), ⁺N(R₁₂)(R₁₃), C(O)OH, C(O)NH₂, NHC(O)NH₂, BH₂, BR₁₂R₁₃, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}—C(O)OH, OC(O)NH—(CH₂)_{q1}C(O)OH, NHCO(CH₂)_{q1}P(O)(OH)₂, NHC(O)(NH)(CH₂)_{q1}C(O)OH, CONH(CH₂)_{q1}—P(O)(OH)₂, NHS(O)₂(CH₂)_{q1}C(O)OH, CO(CH₂)_{q1}S(O)₂(OH), NHS(O)₂NH—(CH₂)_{q1}C(O)OH, OS(O)₂NH(CH₂)_{q1}C(O)OH, NHCO(CH₂)_{q1}S(O)₂(OH), NHP(O)(OH)(NH)—(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁, Ar, ArR₁₂, ArOH, ArNH₂, ArSH, ArNHR₁₂, or (Aa)_{q1}; p₁, p₂ and p₃ are independently 0-30 but are not 0 at the same time; q₁ and q₂ are independently 0-24; Preferably G₃ is lineal or branched, a C₂-C₅₀ polycarboxylacid or a C₂-C₅₀ polyalkylamine, a C₆-C₅₀ oligosaccharide or polysaccharide, a C₆-C₅₀ zwitterionic betaines or zwitterionic poly(sulfobetaine)) (PSB)s that consist of a quaternary ammonium cation and a sulfonate anion, biodegradable polymer (such as composed of poly (lactic/glycolic) acid (PLGA), poly(acrylates), chitosans, copolymer of N-(2-hydroxypropyl)-methacrylamide, poly[2-(methacryloyloxy)ethyl phosphorylcholine] (PMPC), poly-L-glutamic acid, poly(lactide-co-glycolide) (PLG), poly(lactide-co-glycolide), Poly(ethylene glycol)(PEG), poly(propylene glycol)(PPG), poly(lactide-co-glycolide), poly(ethylene glycol)-modified peptides, poly(ethylene glycol)-modified lipids, poly(ethylene glycol)-modified alkylcarboxic acid, poly(ethylene glycol)-modified alkylamine, poly(lactide)-co-glycolide, polysarcosine, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium. More preferably, the formula (I-q) is specifically selected from:

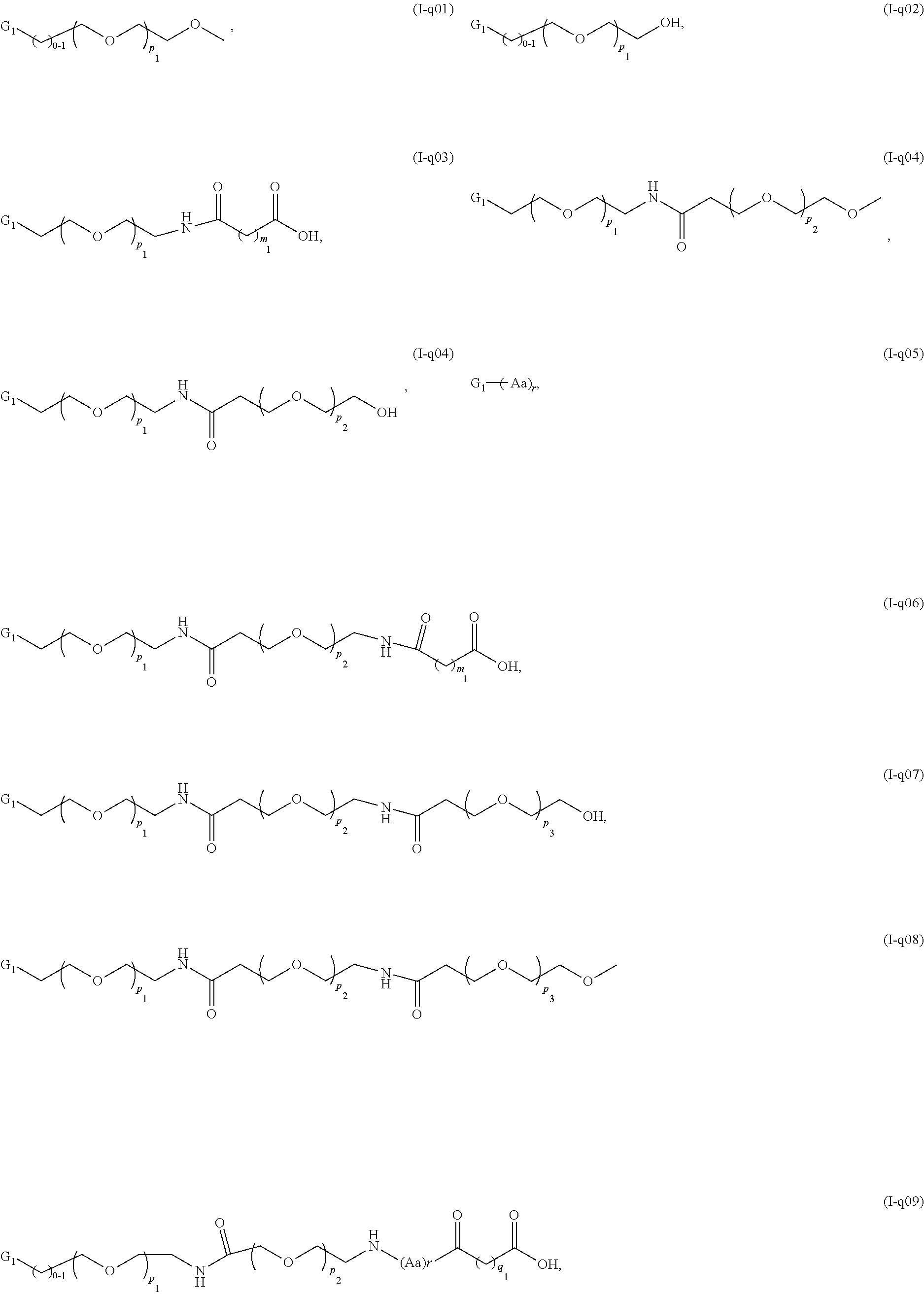

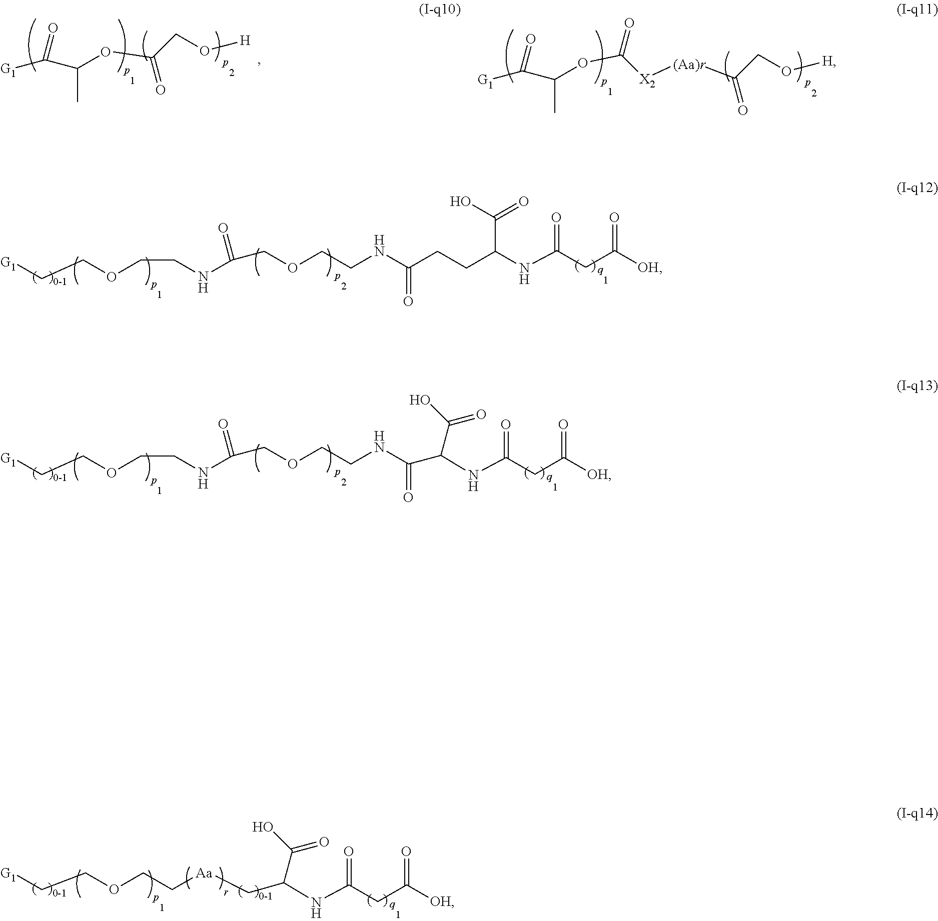

Wherein G₁, p₁, p₂, p₃, Aa, r, X₂, q₁, m₁ are defined the same above.

In yet another aspect of the invention, the binding molecule (T) of the invention may be of any kind presently known, or that become known, molecule that binds to, complexes with or reacts with a moiety of a cell population sought to be therapeutically or otherwise biologically modified. The binding molecule unit acts to deliver the Camptothecin analogs to the particular target cell population with which the binding molecule (T) reacts.

In yet another specific aspect of the invention, the conjugates of CPT analogs to a cell-binding molecule have the formula (IIq-1), (IIq-2), (IIq-3), (IIq-4), (IIq-5), (IIq-6), (IIq-7), (IIq-8) illustrated below:

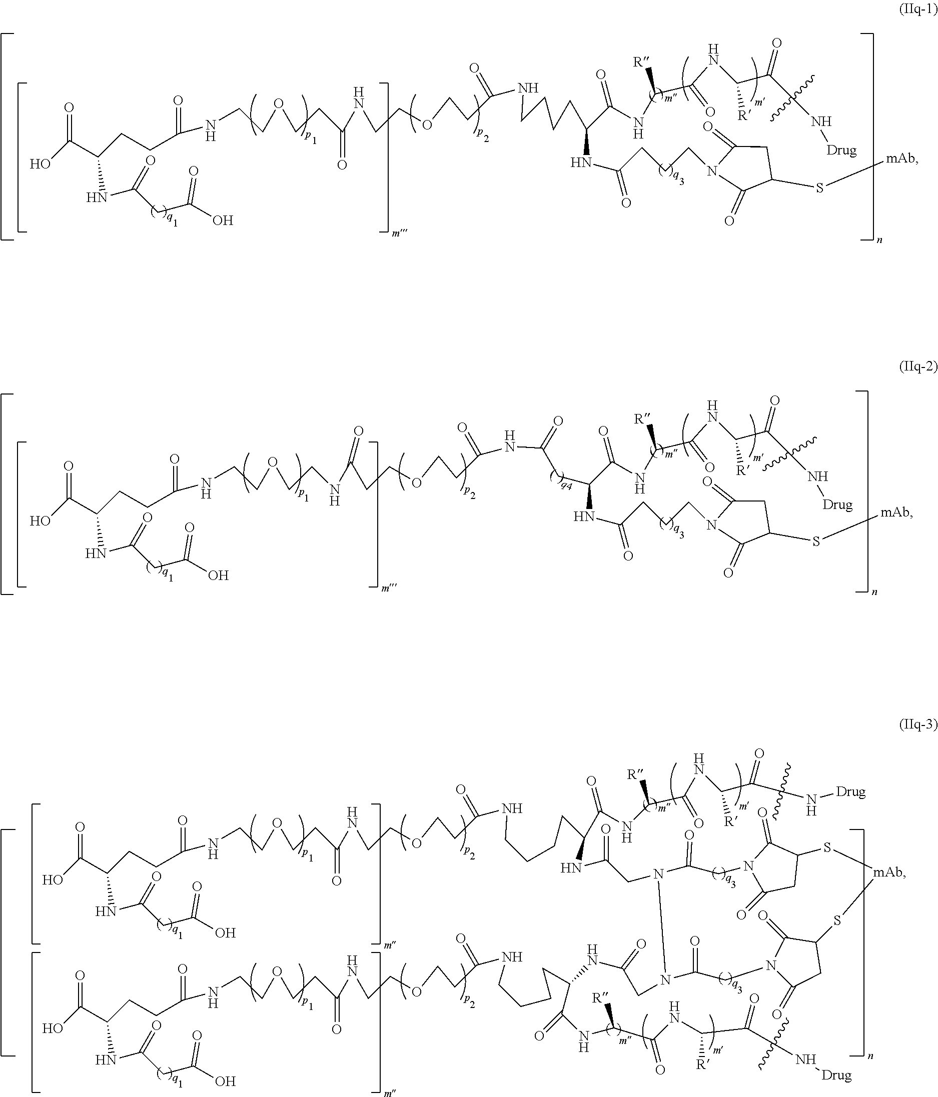

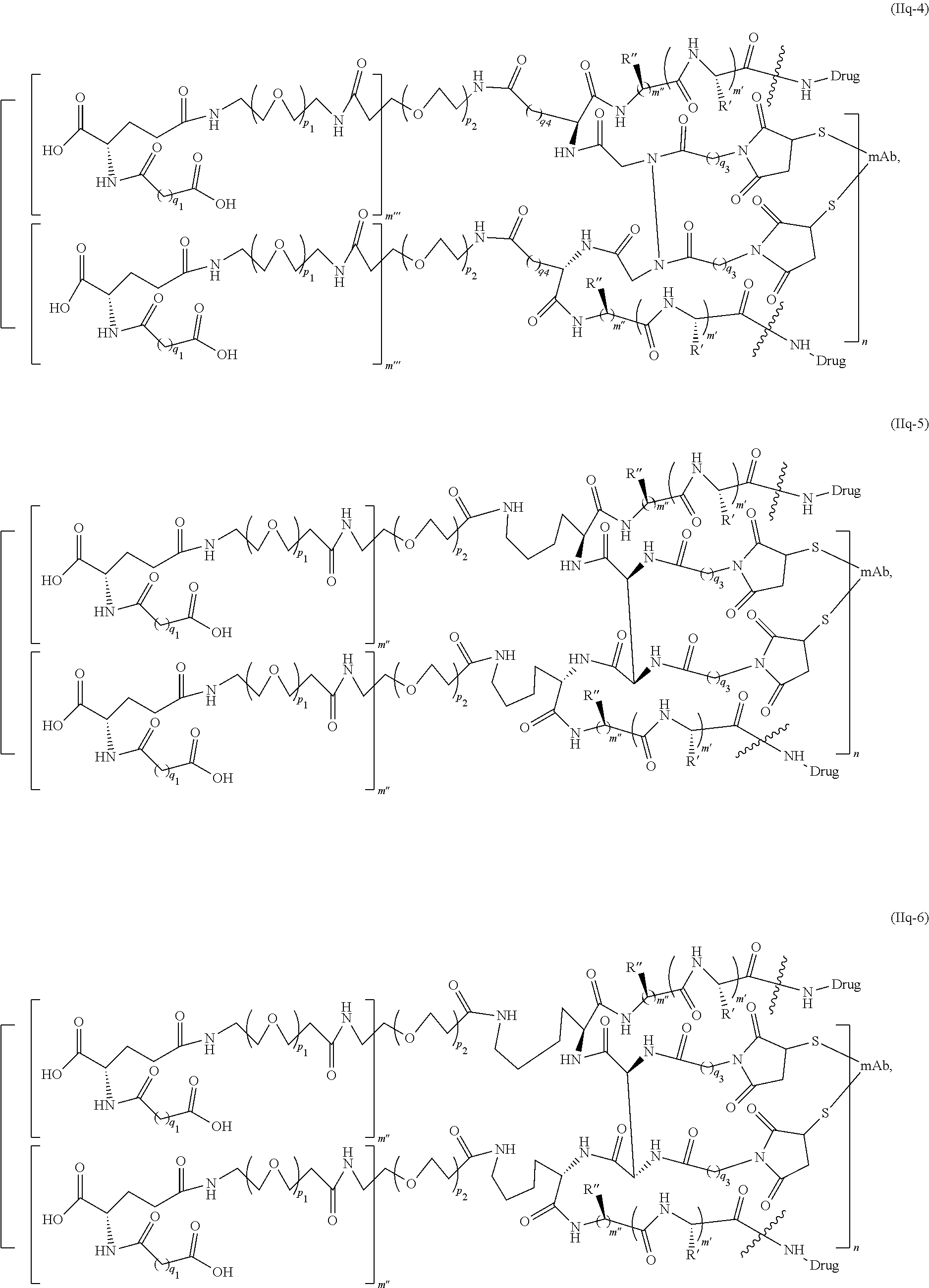

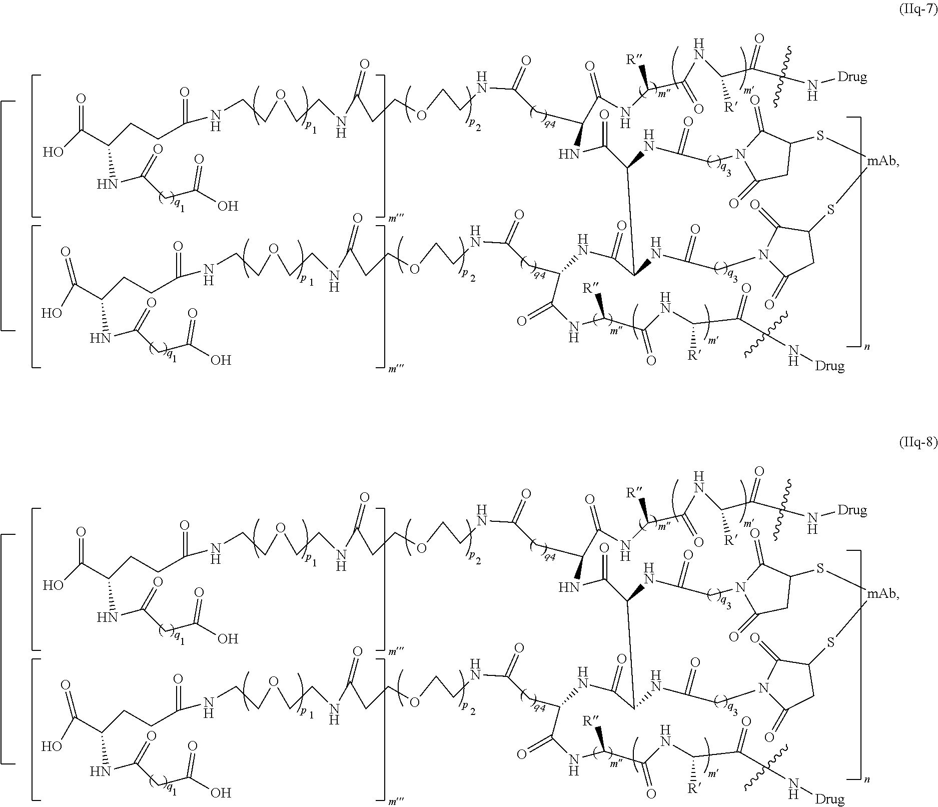

wherein R′ and R″ are independently H, Me, Et, iPr, iBu, Bz(CH₂C₆H₅), CH₂OOH, CH₂CH₂COOH, C₂CONH₂, CH₂CH₂CONH₂, CH₂CH₂CH₂CH₂NH₂, CH₂CH₂SCH₃, CH₂OH, CH₂CH₂CH₂NHC(═NH)NH₂, CH(OH)CH₃, CH₂C₆H₄OH, CH₂C₃N₂H₃; p₁ and p₂ are independently 0˜24; q₁ is 1˜18; q₃ is 0˜6; q₄ is 0˜4; m′ and m″ are independently 0˜6; m′″ is 0 or 1; mAb is a cell-binding molecule, preferably an antibody; NH-Drug here is the compound II-1˜II-61, III-1˜III-51, IV-1˜IV-47, and V-1˜V-61 listed above; n is 1-20, and [symbol] is the site linked to NH-Drug.

The preparation of the conjugates of the formula (IIq-1), (IIq-2), (IIq-3), (IIq-4), (IIq-5), (IIq-6), (IIq-7), (IIq-8) are through reaction of formula (IIq-9), (IIq-10), (IIq-11), (IIq-12), (IIq-13), (IIq-14), (IIq-15), (IIq-16) illustrated below with a cell-binding molecule containing thiols:

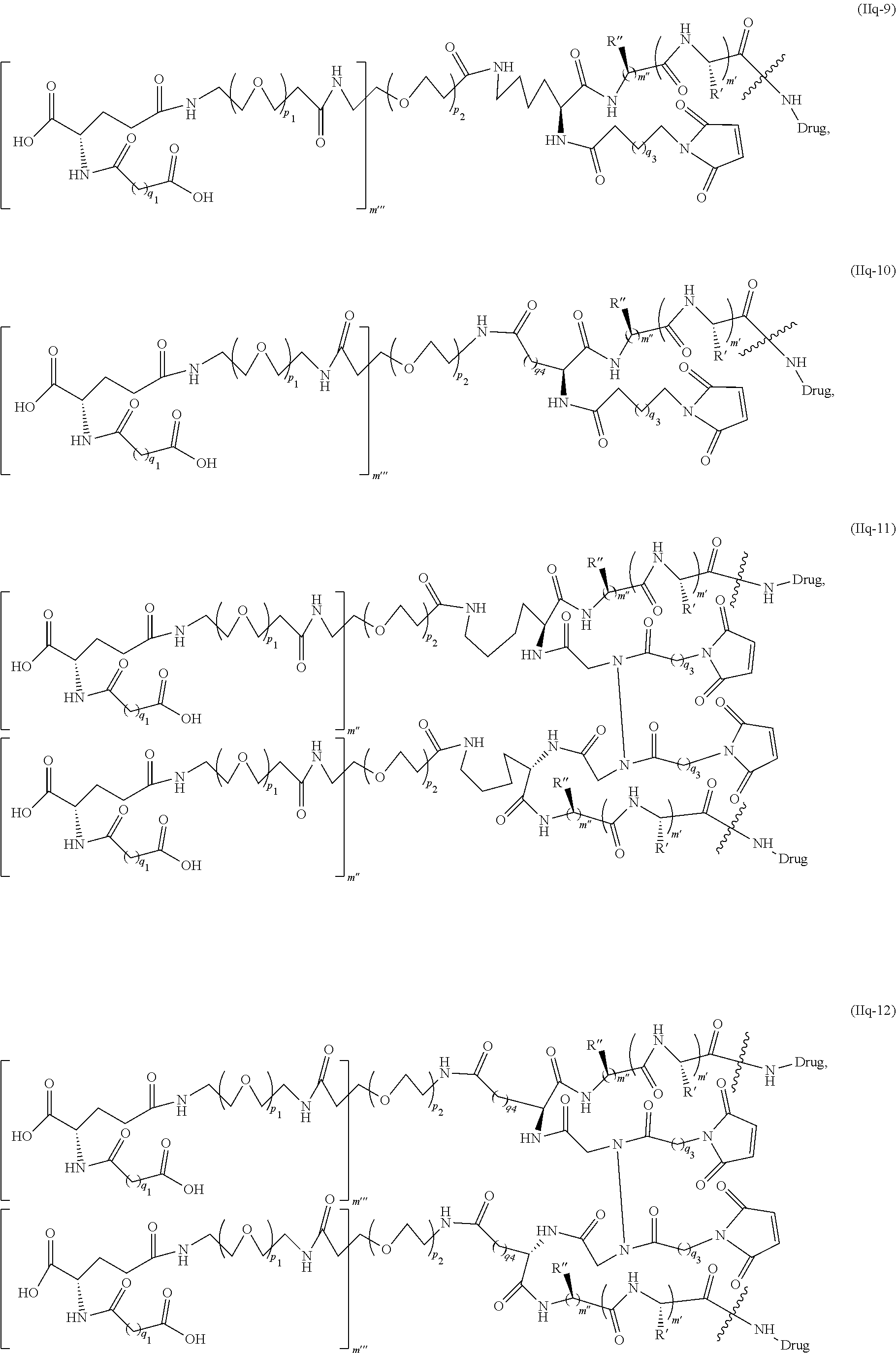

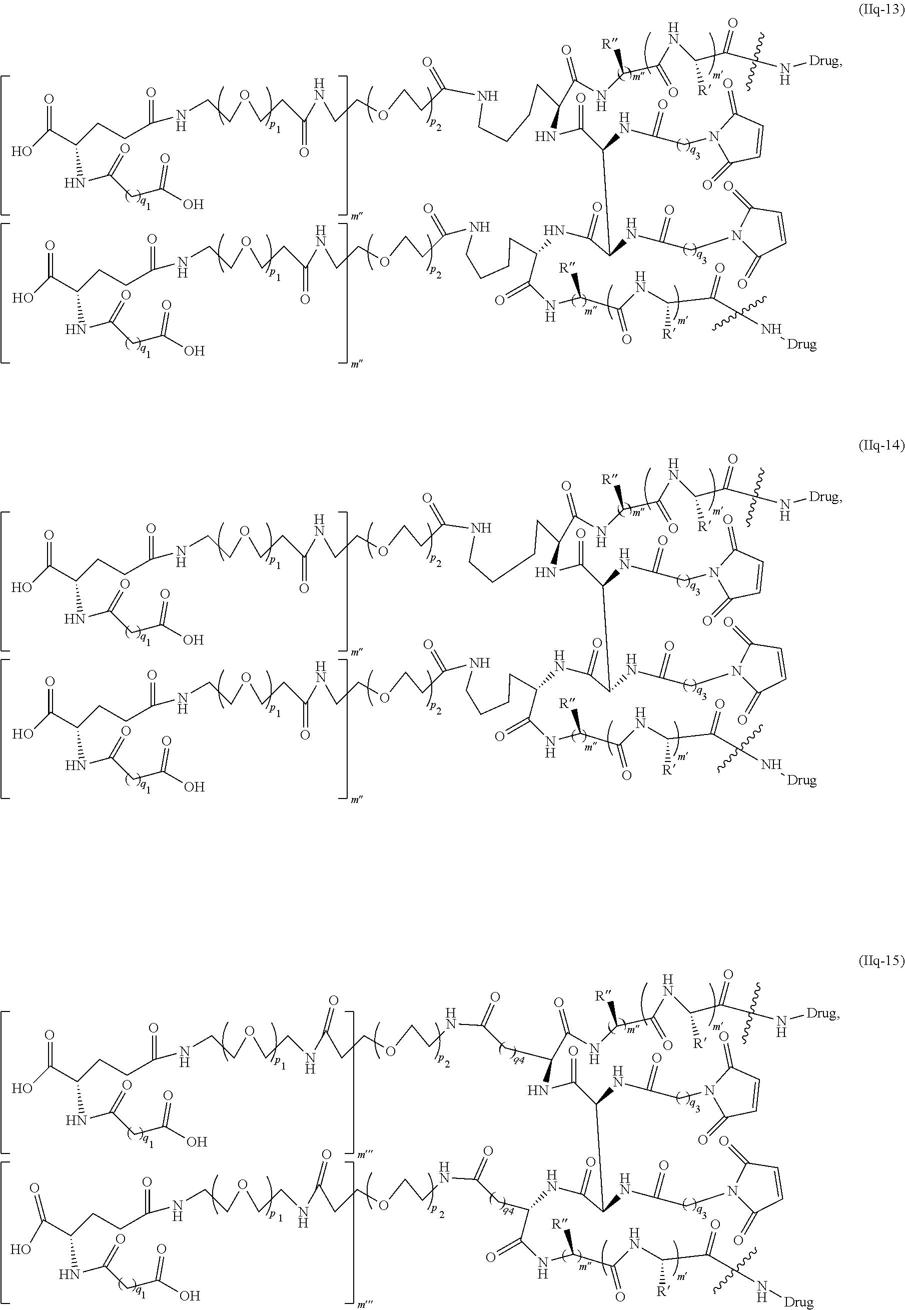

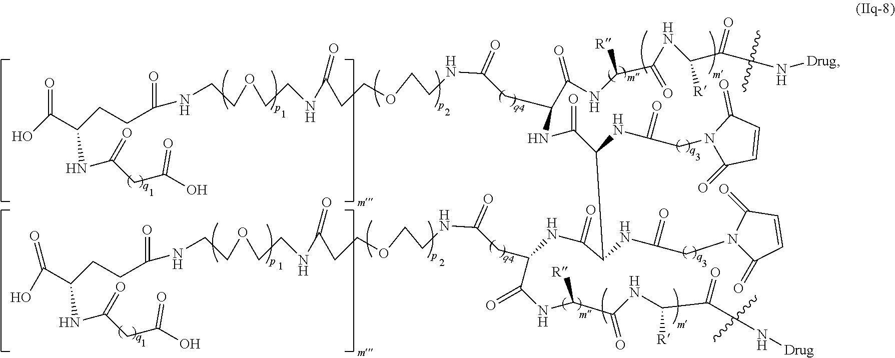

wherein R′ and R″ are independently H, Me, Et, iPr, iBu, Bz (CH₂C₆H₅), CH₂COOH, CH₂CH₂COOH, CH₂CONH₂, CH₂CH₂CONH₂, CH₂CH₂CH₂CH₂NH₂, CH₂CH₂SCH₃, CH₂OH, CH₂CH₂CH₂NHC(═NH)NH₂, CH(OH)CH₃, CH₂C₆H₄OH, CH₂C₃N₂H₃; p₁ and p₂ are independently 0˜24; q₁ is 1˜18; q₃ is 0˜6; q₄ is 0˜4; m′ and m″ are independently 0˜6; m″′ is 0 or 1; NH-Drug here is the compound II-1˜II-61, III-1˜III-51, IV-1˜IV-47, and V-1˜V-61 listed above; and [symbol] is the site linked to NH-Drug.

The free thiols when in a protein, in particular in an antibody, can be generated from a reduction of the inter chain disulfide atoms of the protein by a reduction agent selected from dithiothreitol (DTT), dithioerythritol (DTE), dithiolbutylamine (DTBA), L-glutathione (GSH), tris (2-carboxyethyl) phosphine (TCEP), 2-mercaptoethylamine (β-MEA), or/and beta mercaptoethanol (β-ME, 2-ME) at a buffer solution having pH 5.0˜8.5. The reduction of two or more disulfide residues of the protein with TCEP can be performed simultaneously or prior to the conjugation reaction with Formula (IIq-9), (IIq-10), (IIq-11), (IIq-12), (IIq-13), (IIq-14), (IIq-15), or (IIq-16). The thiols on a protein can also be generated through reaction of amino group (lysine residue) with the Traut's reagent (2-iminothiolane), or γ-thiobutyrolactone. The conjugation of the protein with Formula (IIq-9), (IIq-10), (IIq-11), (IIq-12), (IIq-13), (IIq-14), (IIq-15), or (IIq-16) can be proceeded in one pot (simultaneously) having the Traut reagent (2-iminothiolane), or γ-thiobutyrolactone compound.

The cell-binding agents, T include, but are not limited to, large molecular weight proteins such as, for example, full-length antibodies (polyclonal and monoclonal antibodies); single chain antibodies; fragments of antibodies such as Fab, Fab′, F(ab′)₂, Fᵥ, [Parham, J. Immunol. 131, 2895-2902 (1983)], fragments produced by a Fab expression library, anti-idiotypic (anti-Id) antibodies, CDR's, and epitope-binding fragments of any of the above which immuno-specifically bind to cancer cell antigens, viral antigens or microbial antigens; interferons (such as type I, II, III); peptides; lymphokines such as IL-2, IL-3, IL-4, IL-6, GM-CSF, interferon-gamma (IFN-γ); hormones such as insulin, TRH (thyrotropin releasing hormones), MSH (melanocyte-stimulating hormone), steroid hormones, such as androgens and estrogens, melanocyte-stimulating hormone (MSH); growth factors and colony-stimulating factors such as epidermal growth factors (EGF), granulocyte-macrophage colony-stimulating factor (GM-CSF), transforming growth factors (TGF), such as TGFα, TGFβ, insulin and insulin like growth factors (IGF-I, IGF-II) G-CSF, M-CSF and GM-CSF [Burgess, Immunology Today, 5, 155-158 (1984)]; vaccinia growth factors (VGF); fibroblast growth factors (FGFs); smaller molecular weight proteins, poly-peptide, peptides and peptide hormones, such as bombesin, gastrin, gastrin-releasing peptide; platelet-derived growth factors; interleukin and cytokines, such as interleukin-2 (IL-2), interleukin-6 (IL-6), leukemia inhibitory factors, granulocyte-macrophage colony-stimulating factor (GM-CSF); vitamins, such as folate; apoproteins and glycoproteins, such as transferrin {O'Keefe et al, 260 J. Biol. Chem. 932-937 (1985)}; sugar-binding proteins or lipoproteins, such as lectins; cell nutrient-transport molecules; and small molecular inhibitors, such as prostate-specific membrane antigen (PSMA) inhibitors and small molecular tyrosine kinase inhibitors (TKI), non-peptides or any other cell binding molecule or substance, such as bioactive polymers (Dhar, et al, Proc. Natl. Acad. Sci. 2008, 105, 17356-61); dendrimers (Lee, et al, Nat. Biotechnol. 2005, 23, 1517-26; Almutairi, et al; Proc. Natl. Acad. Sci. 2009, 106, 685-90); nanoparticles (Liong, et al, ACS Nano, 2008, 19, 1309-12; Medarova, et al, Nat. Med. 2007, 13, 372-7; Javier, et al, Bioconjugate Chem. 2008, 19, 1309-12); liposomes (Medinai, et al, Curr. Phar. Des. 2004, 10, 2981-9); viral capsides (Flenniken, et al, Viruses Nanotechnol. 2009, 327, 71-93). In general, monoclonal antibodies are preferred as a cell-surface binding agent if an appropriate one is available.

Preferably, T is selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and/or a cell-binding peptide, protein, or small molecule attached or coated on an albumin, a polymer, a dendrimer, a liposome, a nanoparticle, a vesicle, or on a (viral) capsid.

In further preferably, the cell binding agent/molecule, T is capable of targeting against a tumor cell, a virus infected cell, a microorganism infected cell, a parasite infected cell, an autoimmune disease cell, an activated tumor cells, a myeloid cell, an activated T-cell, an affecting B cell, or a melanocyte, or any disease cells expressing any one of the following antigens or receptors: CD1, CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD12w, CD13, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202(a, b), CD203, CD203c, CD204, CD205, CD206, CD207, CD208, CD209, CD210, CDw210a, CDw210b, CD211, CD212, CD213, CD213a₁, CD213a₂, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR₁, CRGF, CRIPTO, CXCR5, LY64, TDGF1, 4-1BB, APO2, ASLG659, BMPR1B, 4-1BB, 5AC, 5T4 (Trophoblastic glycoprotein, TPBG, 5T4, Wnt-Activated Inhibitory Factor 1 or WAIF1), Adenocarcinoma antigen, AGS-5, AGS-22M6, Activin receptor-like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), BCMA, B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11(C—C motif chemokine 11), CCR4 (C—C chemokine receptor type 4), CCR5, CD3E (epsilon), CEA (Carcino-embryonic antigen), CEACAM3, CEACAM5 (carcino-embryonic antigen), CFD (Factor D), Ch4D5, Cholecystokinin 2 (CCK2R), CLDN18 (Claudin-18), CLDN18.1 (Claudin-18.1), CLDN18.2 (Claudin-18.2), Clumping factor A, cMet, CRIPTO, FCSF1R (Colony stimulating factor 1 receptor), CSF2 (colony stimulating factor 2, Granulocyte-macrophage colony-stimulating factor (GM-CSF)), CSP4, CTLA4 (cytotoxic T-lymphocyte-associated protein 4), CTAA16.88 tumor antigen, CXCR4, C—X—C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxin type-1, E. coli shiga toxin type-2, ED-B, EGFL7 (EGF-like domain-containing protein 7), EGFR, EGFRII, EGFRvIII, Endoglin, Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), fibroblast surface antigen, FCGR1, alpha-Fetoprotein, Fibrin II, beta chain, Fibronectin extra domain-B, FOLR (folate receptor), Folate receptor alpha, Folate hydrolase, Fos-related antigen 1F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor, GP100, GPNMB (Trans-membrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (α4, α_{IIb}β₃, αvβ3, α₄β₇, α5β1, α₆β₄, α₇β₇, αIIβ3, α5β5, αvβ5), Interferon gamma-induced protein, ITGA2, ITGB2, KIR2D, Kappa Ig, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), Melan A/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page4, PAP, Paratope of anti-(N-glycolyl-neuraminic acid), PAX3, PAX5, PCSK9, PDCD1 (PD-1, Programmed cell death protein 1), PDGF-Rα (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI)), Rhesus factor, RANKL, RhoC, Ras mutant, RGS5, ROBO4, Respiratory syncytial virus, RON, ROR1, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF-13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-1 (Trop1), TRP-2 (Trop2), Tyrosinase, VCAM-1, VEGF, VEGF-A, VEGF-2, VEGFR-1, VEGFR2, or vimentin, WT1, XAGE 1, or cells expressing any insulin growth factor receptors, or any epidermal growth factor receptors.

In another specific embodiment, the cell-binding molecule can be a ligand or a receptor agonist selected from: folate derivatives (binding to the folate receptor, a protein over-expressed in ovarian cancer and in other malignancies) (Low, P. S. et al 2008, Acc. Chem. Res. 41, 120-9); glutamic acid urea derivatives (binding to the prostate specific membrane antigen, a surface marker of prostate cancer cells) (Hillier, S. M. et al, 2009, Cancer Res. 69, 6932-40); Somatostatin (also known as growth hormone-inhibiting hormone (GHIH) or somatotropin release-inhibiting factor (SRIF)) or somatotropin release-inhibiting hormone) and its analogues such as octreotide (Sandostatin) and lanreotide (Somatuline) (particularly for neuroendocrine tumors, GH-producing pituitary adenoma, paraganglioma, nonfunctioning pituitary adenoma, pheochromocytomas) (Ginj, M., et al, 2006, Proc. Natl. Acad. Sci. U.S.A. 103, 16436-41); Somatostatin receptor subtypes (sst1, sst2, sst3, sst4, and sst5) in GH-secreting pituitaryadenomas (Reubi J. C., Landolt, A. M. 1984 J. Clin. Endocrinol Metab 59: 1148-51; Reubi J. C., Landolt A. M. 1987 J Clin Endocrinol Metab 65: 65-73; Moyse E, et al, J Clin Endocrinol Metab 61: 98-103), gastroenteropancreatic tumors (Reubi J. C., et al, 1987 J Clin Endocrinol Metab 65: 1127-34; Reubi, J. C, et al, 1990 Cancer Res 50: 5969-77), pheochromocytomas (Epel-baum J, et al 1995 J Clin Endocrinol Metab 80:1837-44; Reubi J. C., et al, 1992 J Clin Endocrinol Metab 74: 1082-9), neuroblastomas (Prevost G, 1996 Neuroendocrinology 63:188-197; Moertel, C. L, et al 1994 Am J Clin Path 102:752-756), medullary thyroid cancers (Reubi, J. C, et al 1991 Lab Invest 64:567-573) small cell lung cancers (Sagman U, et al, 1990 Cancer 66:2129-2133), meningiomas, medulloblastomas, or gliomas (Reubi J. C., et al 1986 J Clin Endocrinol Metab 63: 433-8; Reubi J. C., et al 1987 Cancer Res 47: 5758-64; Fruhwald, M. C, et al 1999 Pediatr Res 45: 697-708), breast carcinomas (Reubi J. C., et al 1990 Int J Cancer 46: 416-20; Srkalovic G, et al 1990 J Clin Endocrinol Metab 70: 661-669), lymphomas (Reubi J. C., et al 1992, Int J Cancer 50: 895-900), renal cell cancers (Reubi J. C., et al 1992, Cancer Res 52: 6074-6078), mesenchymal tumors (Reubi J. C., et al 1996 Cancer Res 56: 1922-31), prostatic (Reubi J. C., et al 1995, J. Clin. Endocrinol Metab 80: 2806-14; et al 1989, Prostate 14:191-208; Halmos G, et al J. Clin. Endo-crinol Metab 85: 2564-71), ovarian (Halmos, G, et al, 2000 J Clin Endocrinol Metab 85: 3509-12; Reubi J. C., et al 1991 Am J Pathol 138:1267-72), gastric (Reubi J. C., et al 1999, Int J Cancer 81: 376-86; Miller, G. V, 1992 Br J Cancer 66: 391-95), hepatocellular (Kouroumalis E, et al 1998 Gut 42: 442-7; Reubi J. C., et al 1999 Gut 45: 66-774) and nasopharyngeal carcinomas (Loh K. S, et al, 2002 Virchows Arch 441: 444-8); Aromatic sulfonamides (specific to carbonic anhydrase IX) (a marker of hypoxia and of renal cell carcinoma) (Neri, D., et al, Nat. Rev. Drug Discov. 2011, 10, 767-7); Pituitary adenylate cyclase activating peptides (PACAP) (PAC1) for pheochromocytomas and paragangliomas; Vasoactive intestinal peptides (VIP) and their receptor subtypes (VPAC1, VPAC2); α-Melanocyte-stimulating hormone (α-MSH) receptors; Cholecystokinin (CCK)/gastrin receptors and their receptor subtypes (CCK1 (formerly CCK-A) and CCK2; Bombesin(Pyr-Gln-Arg-Leu-Gly-Asn-Gln-Trp-Ala-Val-Gly-His-Leu-Met-NH₂)/gastrin-releasing peptide (GRP) and their receptor subtypes (BB1, GRP receptor subtype (BB2), the BB3 and BB4) (Ohlsson, B., et al, 1999, Scand. J. Gastroenterology 34(12): 1224-9; Weber. H. C., 2009, Cur. Opin. Endocri. Diab. Obesity 16(1): 66-71, Gonzalez N, et al, 2008, Cur. Opin. Endocri. Diab. Obesity 15(1), 58-64); Neurotensin receptors and its receptor subtypes (NTR1, NTR2, NTR3); Substance P receptors and their receptor subtypes (such as NK1 receptor for Glial tumors, Hennig I. M., et al 1995 Int. J. Cancer 61, 786-792); Neuropeptide Y (NPY) receptors and its receptor subtypes (Y1-Y6); Homing Peptides include RGD (Arg-Gly-Asp), NGR (Asn-Gly-Arg), the dimeric and multimeric cyclic RGD peptides (e.g. cRGDfV) (Laakkonen P, Vuorinen K. 2010, Integr Biol (Camb). 2(7-8): 326-337; Chen K, Chen X. 2011, Theranostics. 1:189-200; Garanger E, et al, Anti-Cancer Agents Med Chem. 7 (5): 552-558; Kerr, J. S. et al, Anticancer Research, 19(2A), 959-968; Thumshirn, G, et al, 2003 Chem. Eur. J. 9, 2717-2725), and TAASGVRSMH or LTLRWVGLMS (chondroitin sulfate proteoglycan NG2 receptor) and F3 peptides (31 amino acid peptide that binds to cell surface-expressed nucleolin receptor) (Zitzmann, S., 2002 Cancer Res., 62, 18, pp. 5139-5143, Temminga, K., 2005, Drug Resistance Updates, 8, 381-402; P. Laakkonen and K. Vuorinen, 2010 Integrative Biol, 2(7-8), 326-337; M. A. Burg, 1999 Cancer Res., 59(12), 2869-2874; K. Porkka, et al 2002, Proc. Nat. Acad. Sci. USA 99(11), 7444-9); Cell Penetrating Peptides (CPPs) (Nakase I, et al, 2012, J. Control Release. 159(2), 181-188); Peptide Hormones, such as luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, e.g. buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloro-Phe-D-3-(3-pyridyl)Ala-Ser-Tyr-D-Cit-Leu-Arg-Pro-D-Ala-NH2), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH2) (Thundimadathil, J., J. Amino Acids, 2012, 967347, doi:10.1155/2012/967347; Boccon-Gibod, L.; et al, 2011, Therapeutic Advances in Urology 3(3): 127-140; Debruyne, F., 2006, Future Oncology, 2(6), 677-696; Schally A. V; Nagy, A. 1999 Eur J Endocrinol 141:1-14; Koppan M, et al 1999 Prostate 38:151-158); and Pattern Recognition Receptors (PRRs), such as Toll-like receptors (TLRs), C-type lectins and Nodlike Receptors (NLRs) (Fukata, M., et al, 2009, Semin. Immunol. 21, 242-253; Maisonneuve, C., et al, 2014, Proc. Natl. Acad. Sci. U.S.A 111, 1-6; Botos, I., et al, 2011, Structure 19, 447-459; Means, T. K., et al, 2000, Life Sci. 68, 241-258) that range in size from small molecules (imiquimod, guanisine and adenosine analogs) tolarge and complex biomacromolecules such as lipopolysaccharide (LPS), nucleic acids (CpG DNA, polyL:C) and lipopeptides (Pam3CSK4) (Kasturi, S. P., et al, 2011, Nature 470, 543-547; Lane, T., 2001, J. R. Soc. Med. 94, 316; Hotz, C., and Bourquin, C., 2012, Oncoimmunology 1, 227-228; Dudek, A. Z., et al, 2007, Clin. Cancer Res. 13, 7119-25); Calcitonin receptors which is a 32-amino-acid neuropeptide involved in the regulation of calcium levels largely through its effects on osteoclasts and on the kidney (Zaidi M, et al, 1990 Crit Rev Clin Lab Sci 28, 109-174; Gorn, A. H., et al 1995 J Clin Invest 95:2680-91); And integrin receptors and their receptor subtypes (such as αvβ₁, αvβ₃, αvβ₅, αvβ₆, α₆β₄, α₇β₁, α_{L}β₂, α_{IIb}β₃, etc.) which generally play important roles in angiogenesis are expressed on the surfaces of a variety of cells, in particular, of osteoclasts, endothelial cells and tumor cells (Ruoslahti, E. et al, 1994 Cell 77, 477-8; Albelda, S. M. et al, 1990 Cancer Res., 50, 6757-64). Short peptides, GRGDSPK and Cyclic RGD pentapeptides, such as cyclo(RGDfV) (L1) and its derives [cyclo(-N(Me)R-GDfV), cyclo(R-Sar-DfV), cyclo-(RG-N(Me)D-fV), cyclo(RGD-N(Me)f-V), cyclo(RGDf-N(Me)V-)(Cilengitide)] have shown high binding affinities of the intergrin receptors (Dechantsreiter, M. A. et al, 1999 J. Med. Chem. 42, 3033-40, Goodman, S. L., et al, 2002 J. Med. Chem. 45, 1045-51).

The cell-binding molecule/ligands or cell receptor agonists can be Ig-based and non-Ig-based protein scaffold molecules. The Ig-Based scaffolds can be selected, but not limited, from Nanobody (a derivative of VHH (camelid Ig)) (Muyldermans S., 2013 Annu Rev Biochem. 82, 775-97); Domain antibodies (dAb, a derivative of VH or VL domain) (Holt, L. J, et al, 2003, Trends Biotechnol. 21, 484-90); Bispecific T cell Engager (BiTE, a bispecific diabody) (Baeuerle, P. A, et al, 2009, Curr. Opin. Mol. Ther. 11, 22-30); Dual Affinity ReTargeting (DART, a bispecific diabody) (Moore P. A. P, et al. 2011, Blood 117(17), 4542-51); Tetravalent tandem antibodies (TandAb, a dimerized bispecific diabody) (Cochlovius, B, et al. 2000, Cancer Res. 60(16):4336-4341). The Non-Ig scaffolds can be selected, but not limited, from Anticalin (a derivative of Lipocalins) (Skerra A. 2008, FEBS J., 275(11): 2677-83; Beste G, et al, 1999 Proc. Nat. Acad. USA. 96(5):1898-903; Skerra, A. 2000 Biochim Biophys Acta, 1482(1-2): 337-50; Skerra, A. 2007, Curr Opin Biotechnol. 18(4): 295-304; Skerra, A. 2008, FEBS J. 275(11):2677-83); Adnectins (10th FN3 (Fibronectin)) (Koide, A, et al, 1998 J. Mol. Biol., 284(4):1141-51; Baton V, 2002, Protein Eng. 15(12): 1015-20; Tolcher, A. W, 2011, Clin. Cancer Res. 17(2): 363-71; Hackel, B. J, 2010, Protein Eng. Des. Sel. 23(4): 211-19); Designed Ankyrin Repeat Proteins (DARPins) (a derivative of ankrin repeat (AR) proteins) (Boersma, Y. L, et al, 2011 Curr Opin Biotechnol. 22(6): 849-57), e.g. DARPin C9, DARPin Ec4 and DARPin E69_LZ3_E01 (Winkler J, et al, 2009 Mol Cancer Ther. 8(9), 2674-83; Patricia M-K. M., et al, Clin Cancer Res. 2011; 17(1):100-10; Boersma Y. L, et al, 2011 J. Biol. Chem. 286(48), 41273-85); Avimers (a domain A/low-density lipoprotein (LDL) receptor) (Boersma Y. L, 2011 J. Biol. Chem. 286(48): 41273-41285; Silverman J, et al, 2005 Nat. Biotechnol., 23(12):1556-61).

Examples of the small molecule structures of the cell-binding molecules/ligands or cell receptor agonists of the patent application are the following: LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αᵥβ₃ integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor), LB23 (for an androgen receptor), LB24 (Cilengitide/cyclo(-RGDfV-) for an αᵥ intergrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a nucleoside analog), LB53 (Erlotinib analog) and LB54 (Lapatinib analog) which are shown in the following structures:

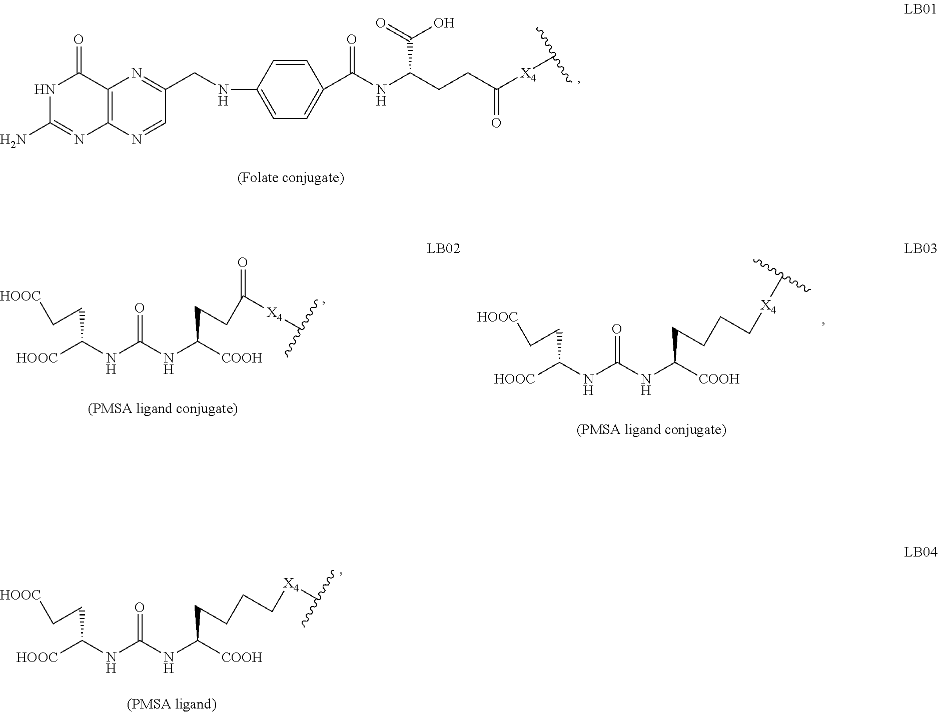

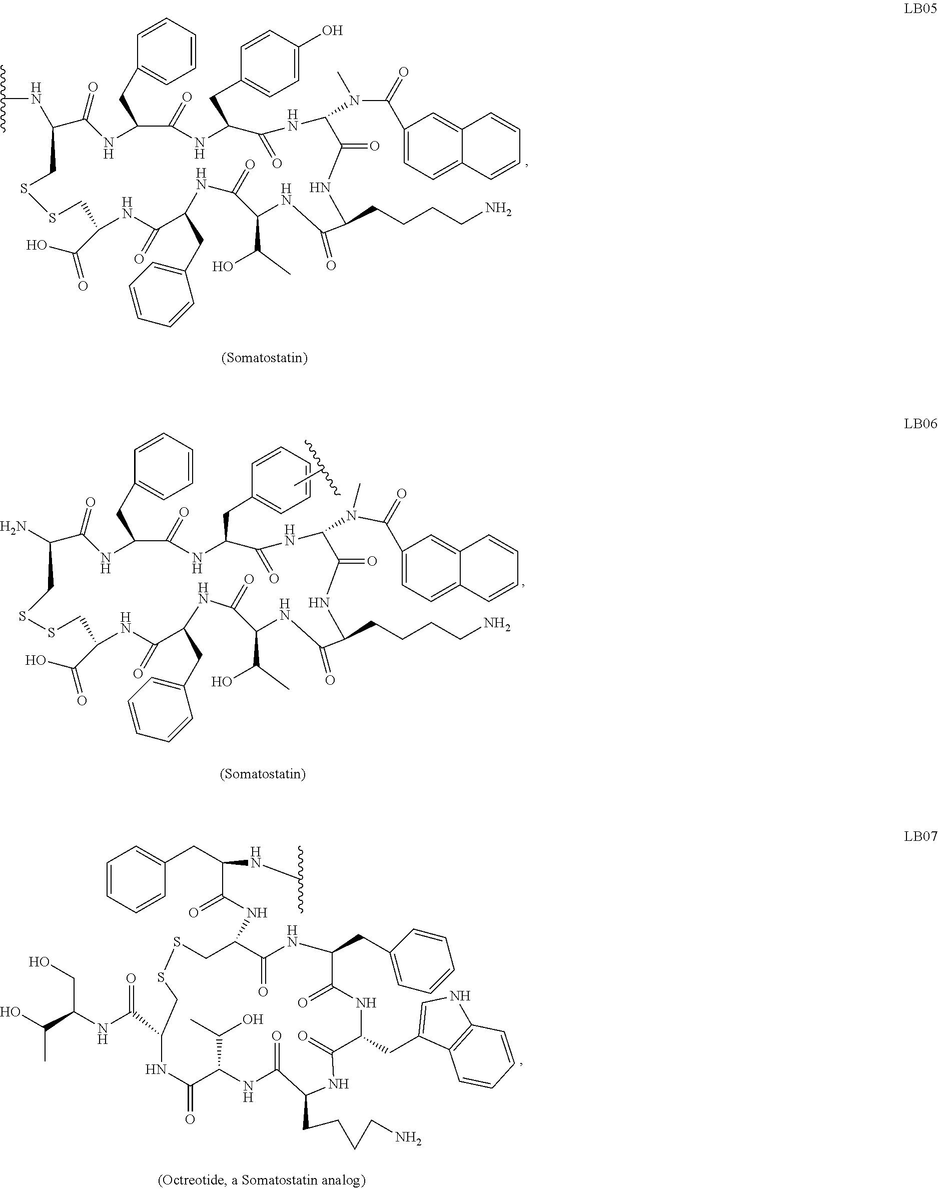

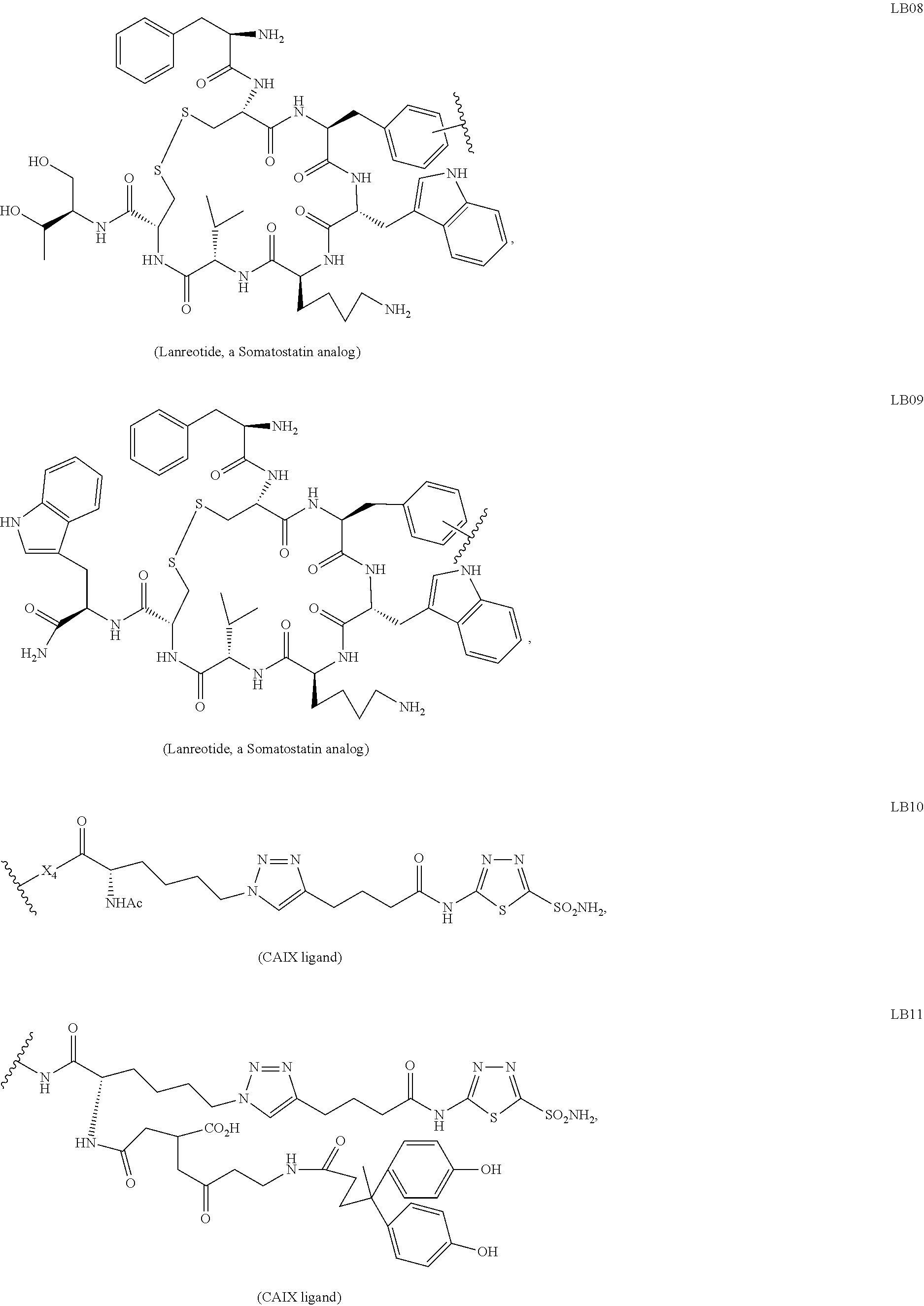

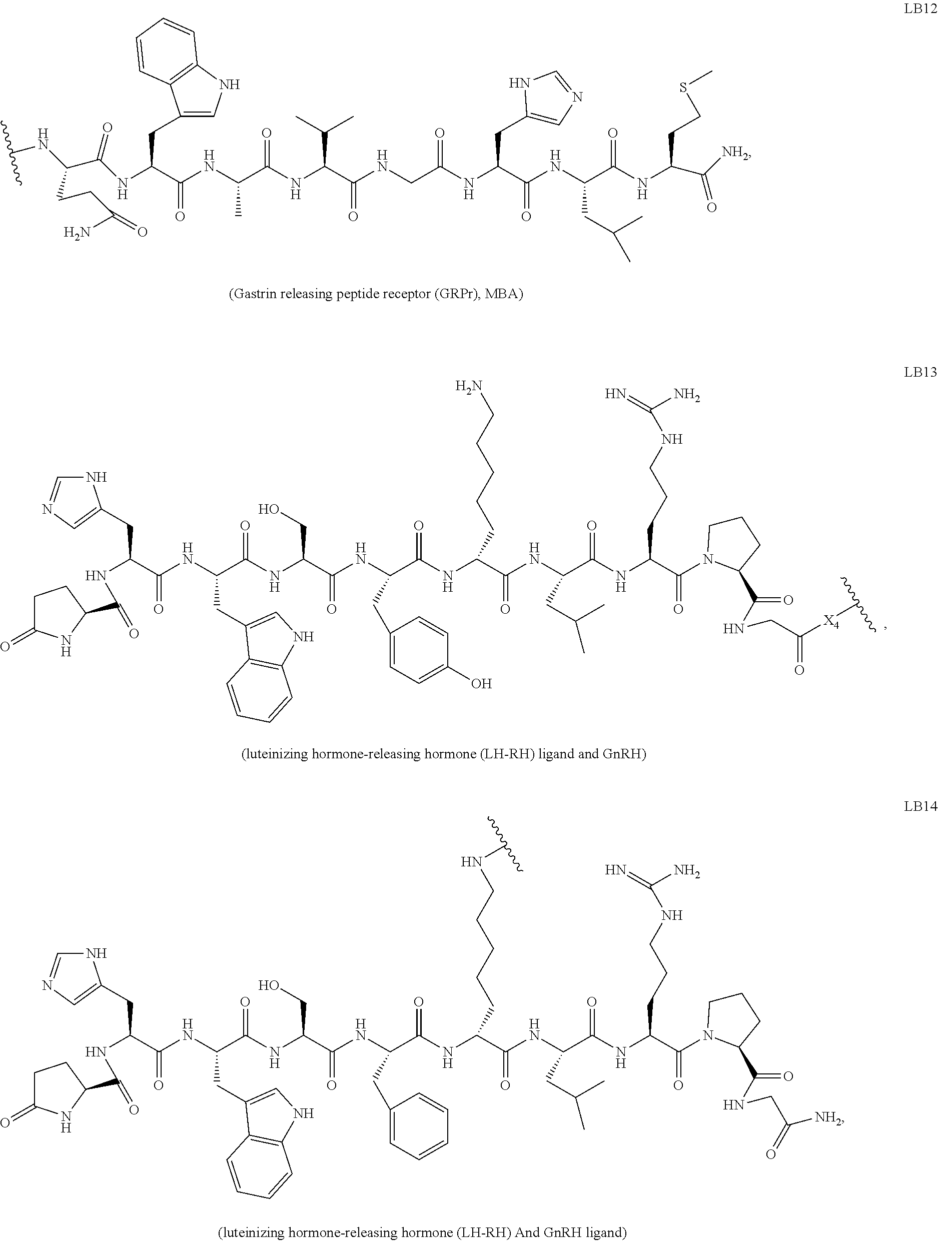

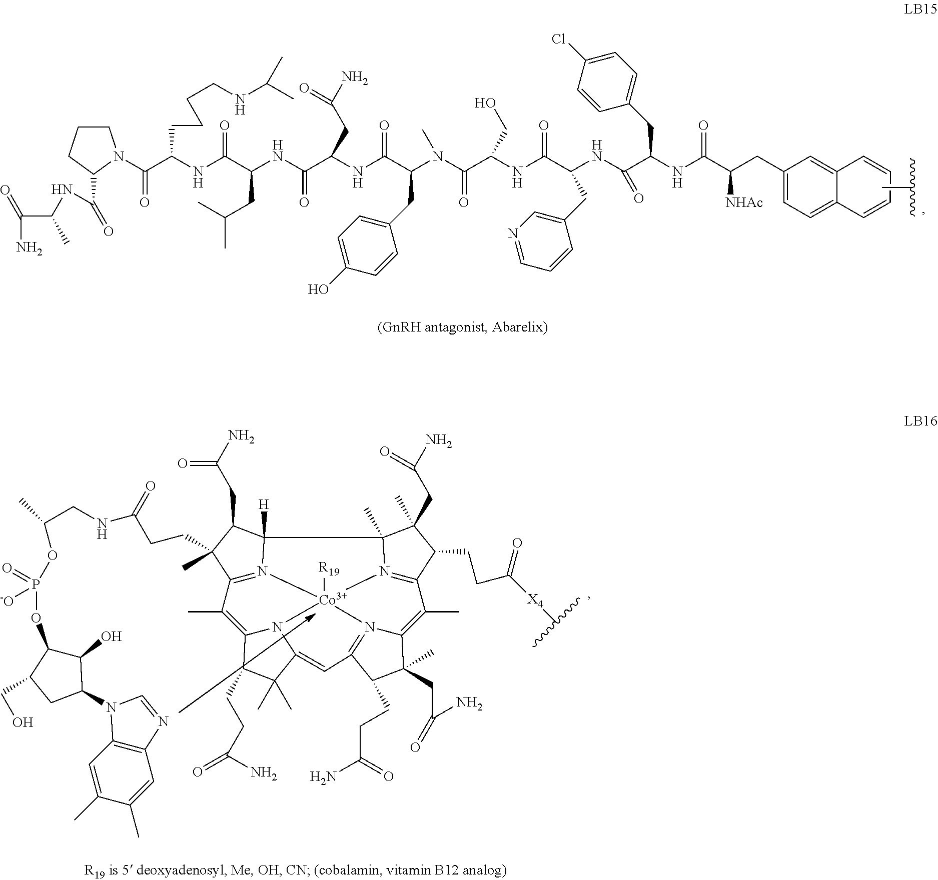

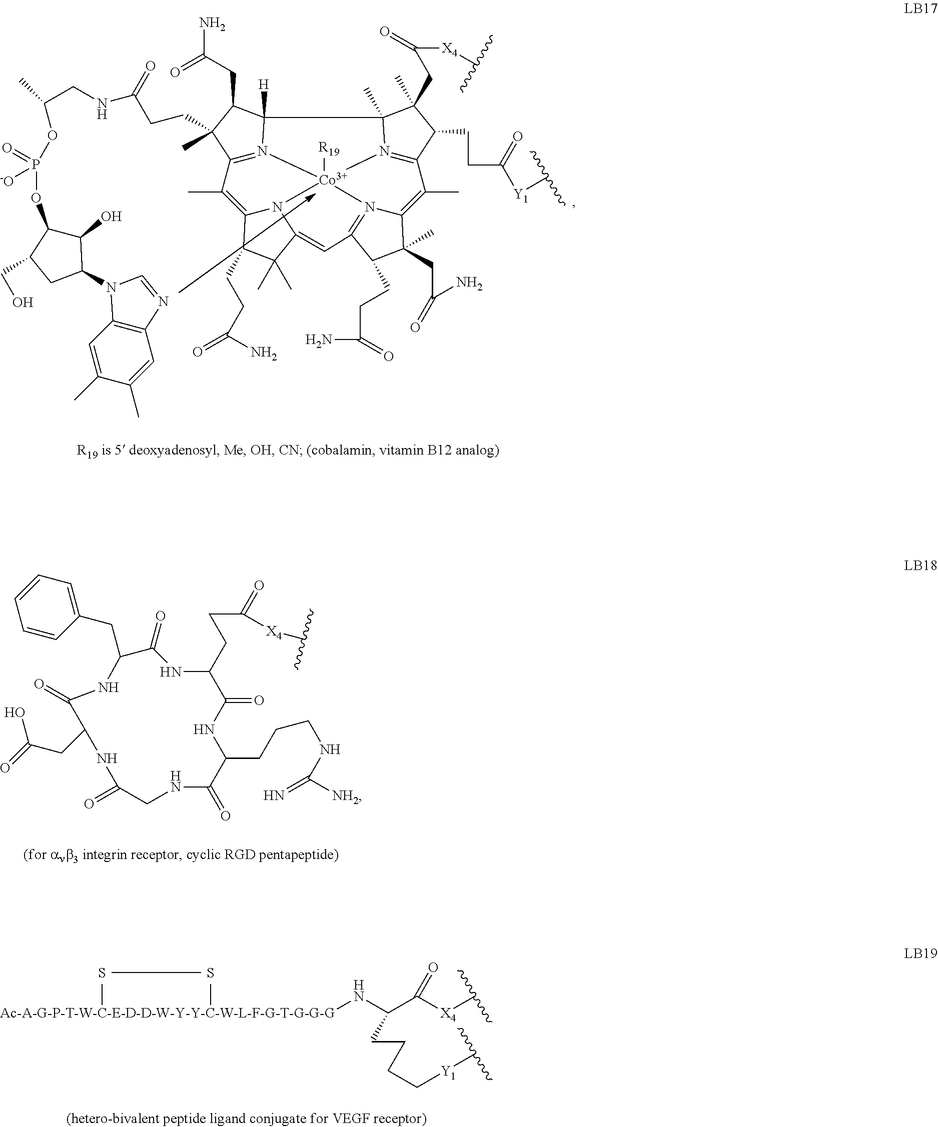

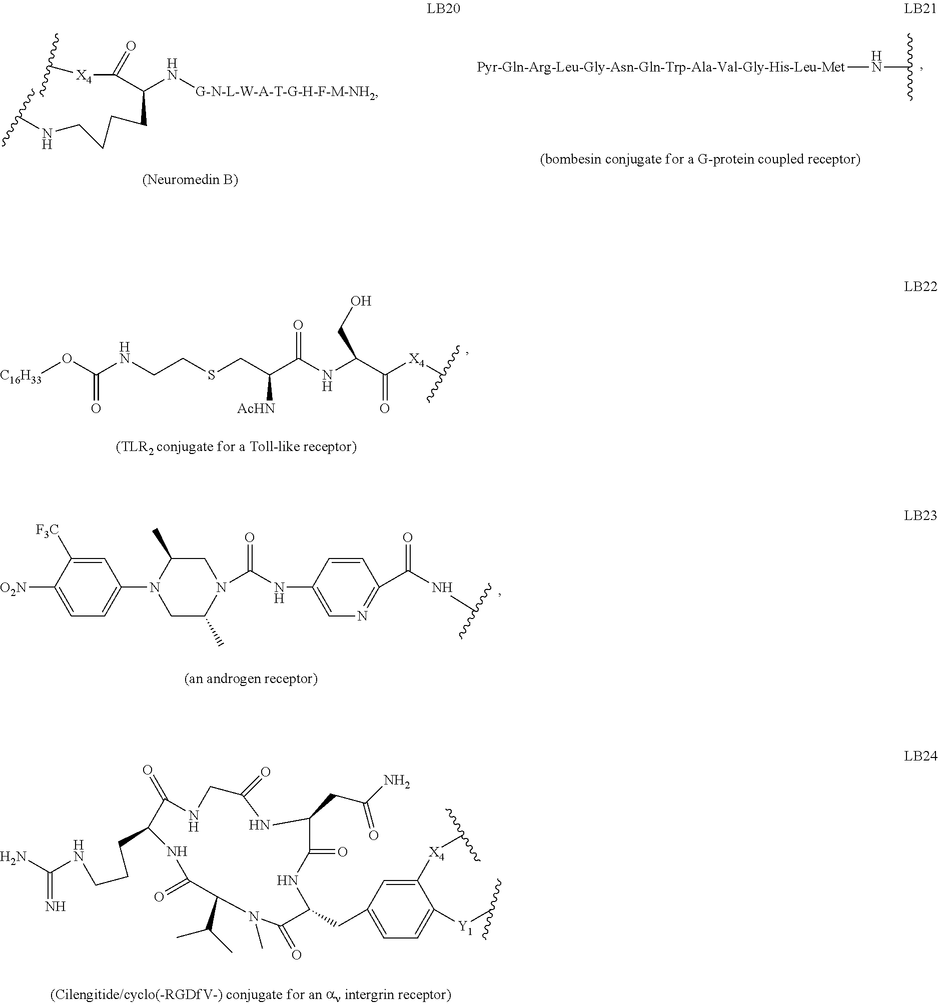

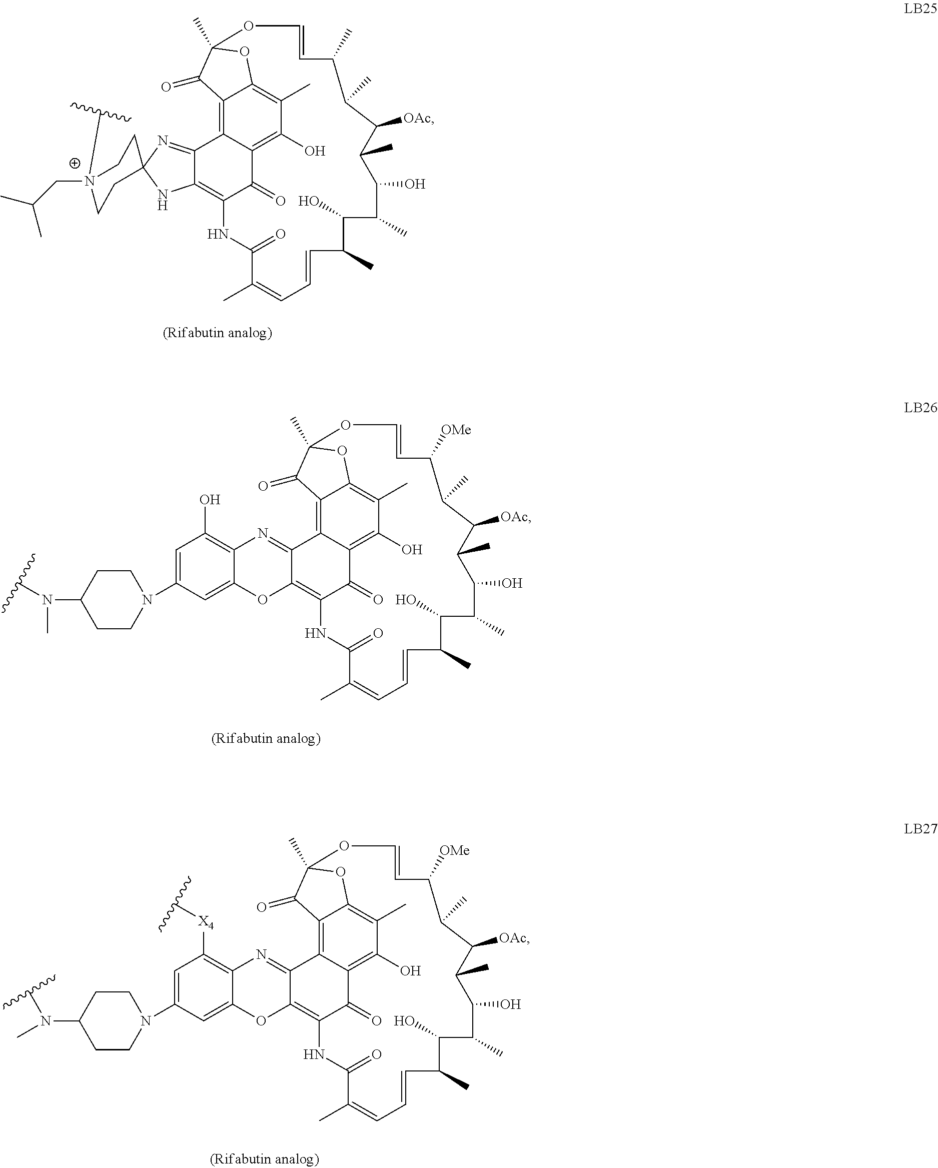

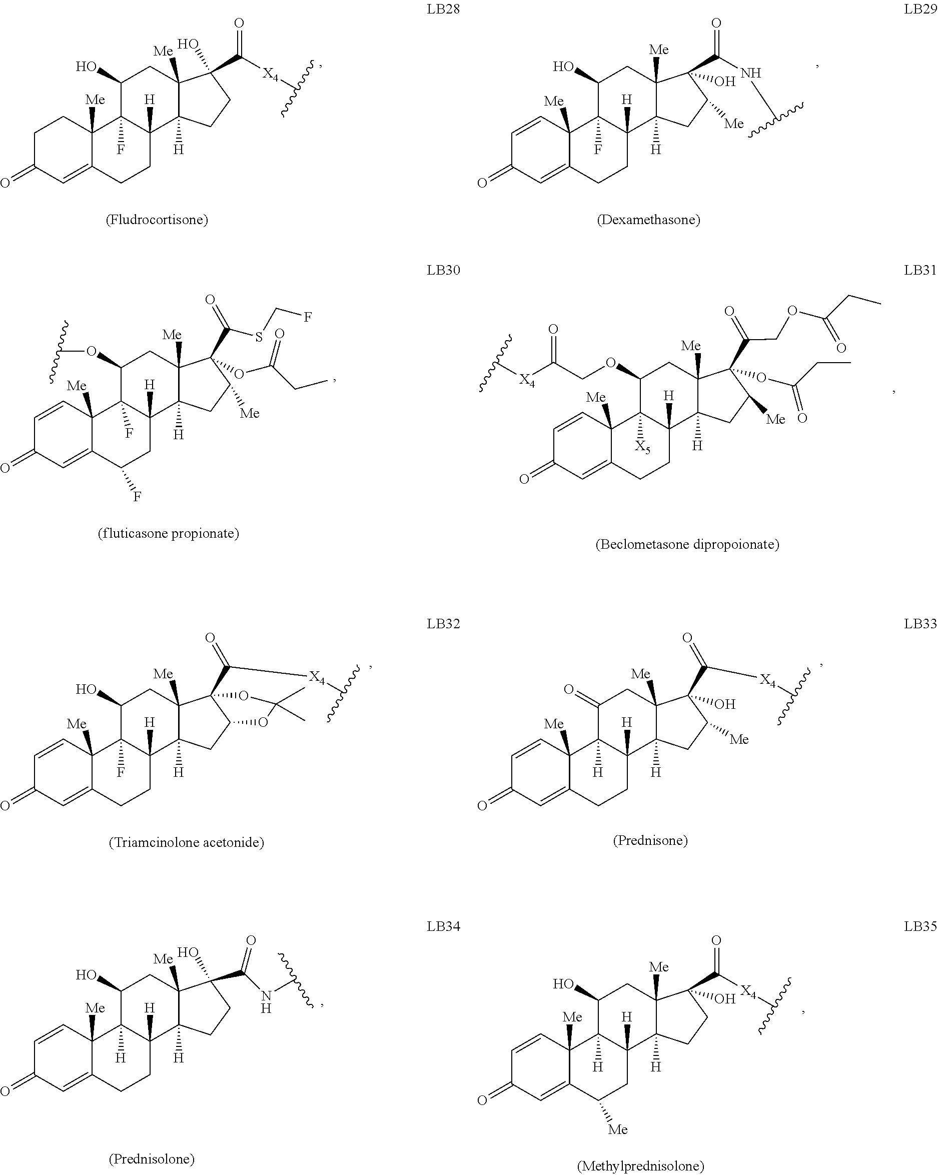

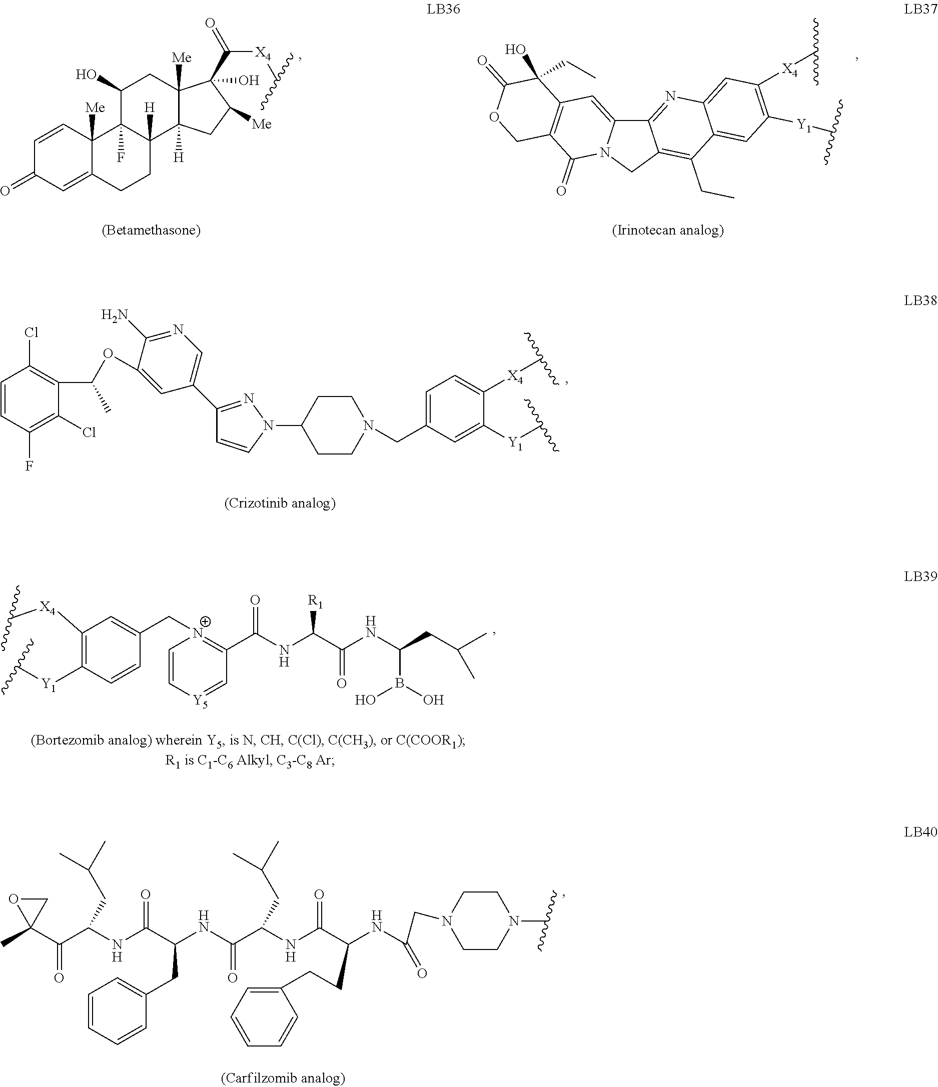

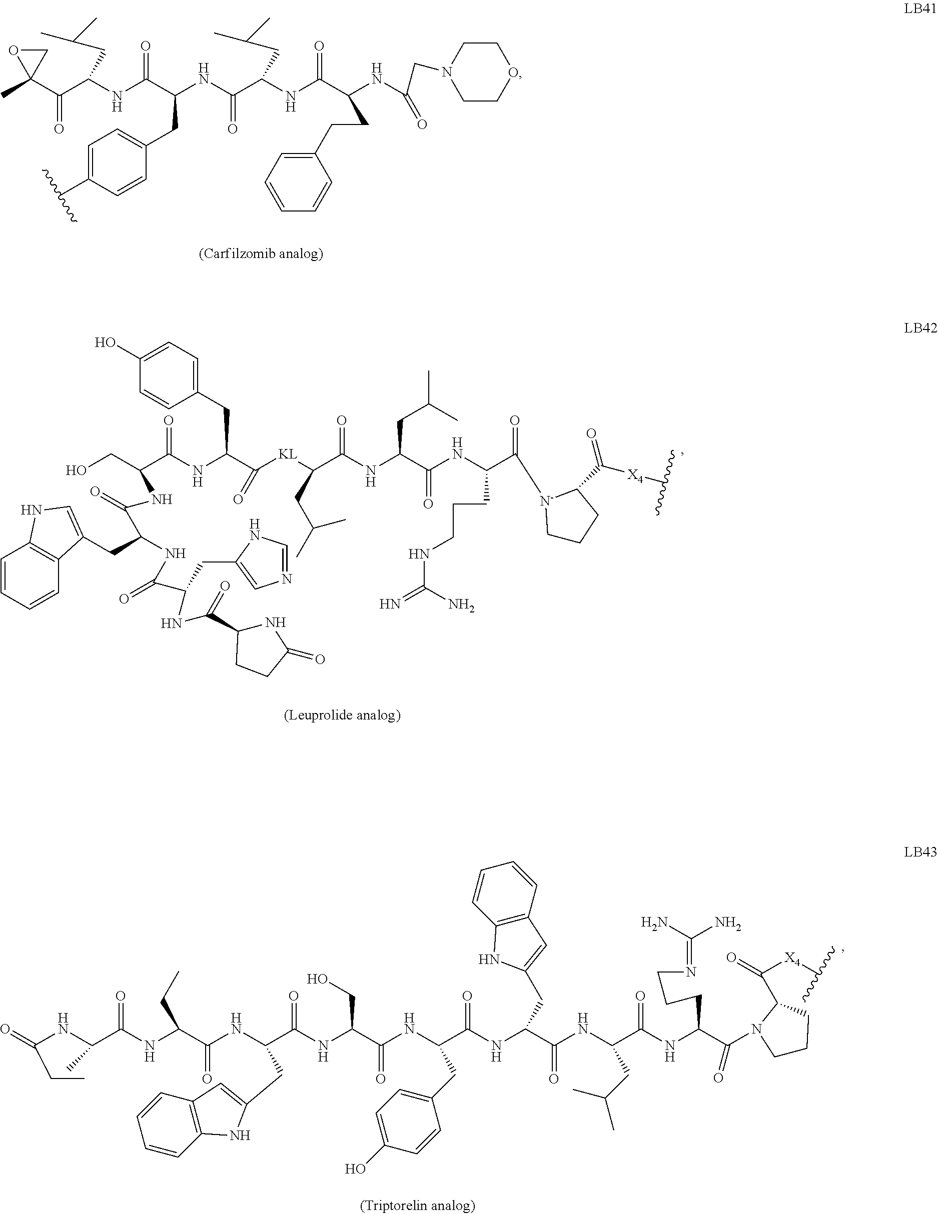

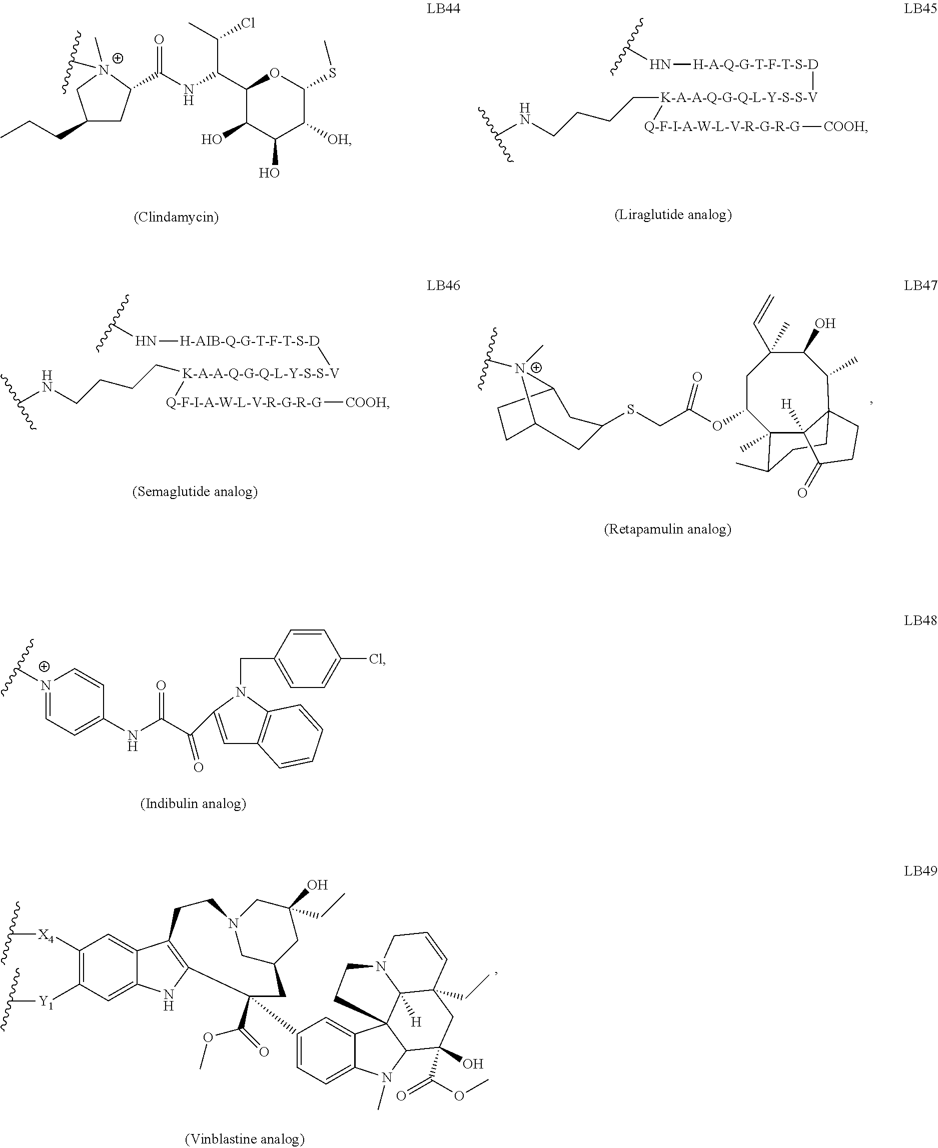

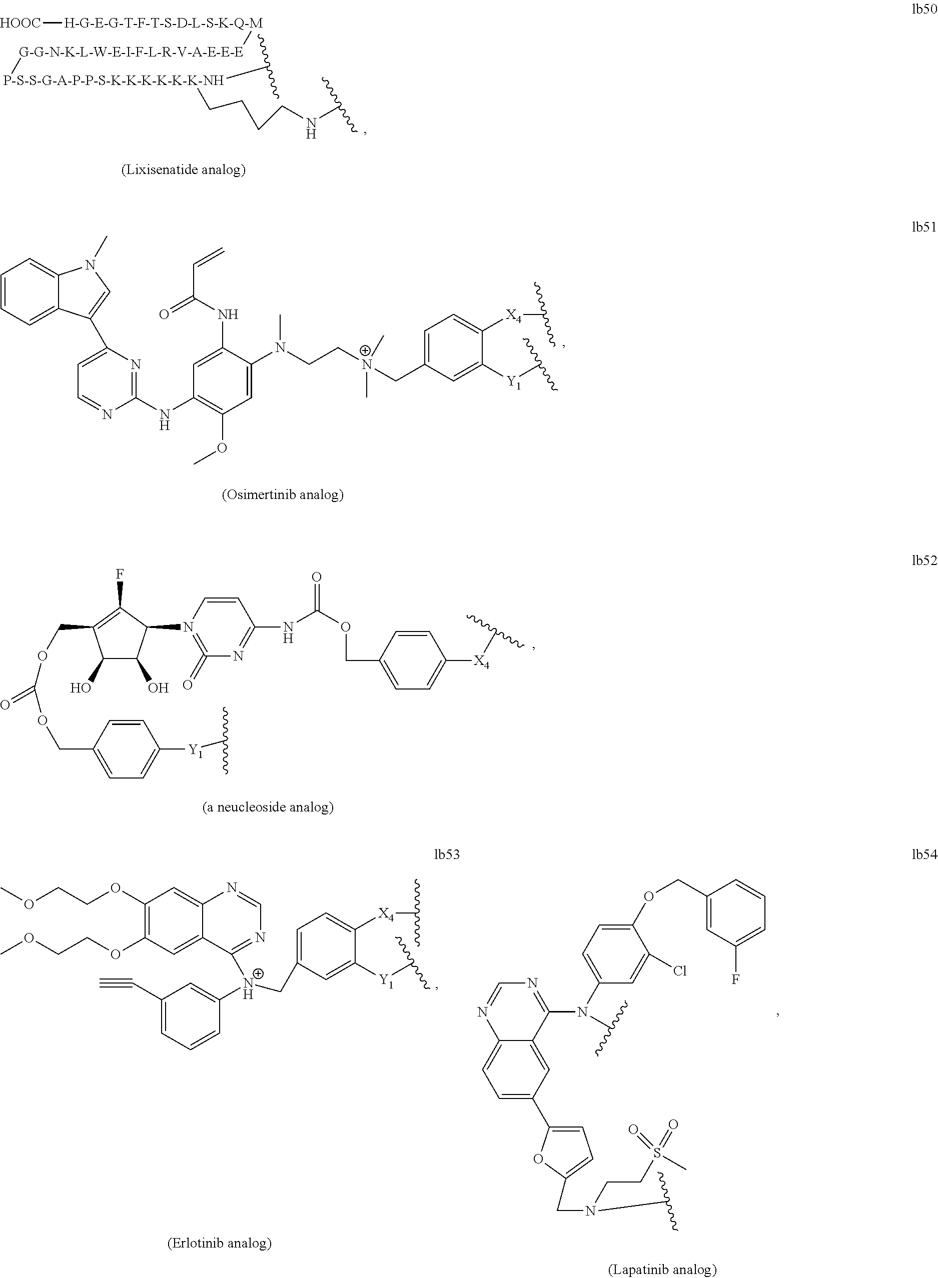

wherein “ [symbol] ” is the site to link the side chain linker of the present patent; X₄, and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁; X₁ is H, CH₂, OH, O, C(O), C(O)NH, C(O)N(R₁), R₁, NHR₁, NR₁, C(O)R₁ or C(O)O; X₅ is H, CH₃, F, or Cl; M₁ and M₂ are independently H, Na, K, Ca, Mg, NH₄, N(R¹R^{1′}R²R³); R¹, R^{1′}, R² and R³ are defined in Formula (I).

Preparation of the Conjugates

In another aspect of the present invention, the camptothecin analog is preferably synthesized containing a linker L and a reactive group Lv, represented by Formula (VI), (VII), (VIII), (IX) and (X) which can readily react to a cell-binding molecule T, or to a modified cell-binding molecule T to form a conjugate of Formula (I), (II), (III), (IV) and (V) respectively:

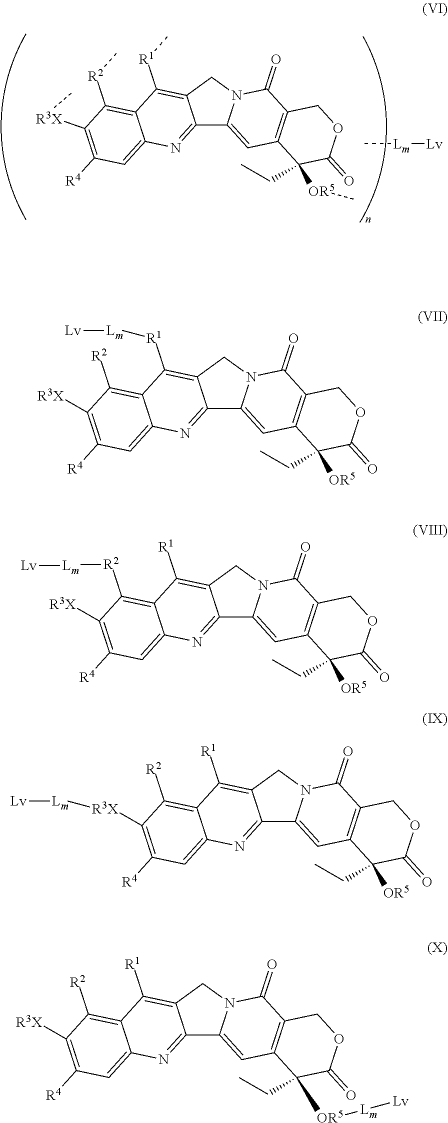

wherein R₁, R₂, R₃, R₄, R₅, L, X and m are defined the same as Formula (I) above;

Lv is a reacting group that can react with a thiol, amine, carboxylic acid, selenol, phenol or hydroxyl group on a cell-binding molecule. Such reacting groups are, but are not limited to, a halide (e.g., fluoride, chloride, bromide, and iodide), maleimide, methanesulfonyl (mesyl), toluenesulfonyl (tosyl), trifluoromethyl-sulfonyl (triflate), trifluoro-methylsulfonate, nitrophenoxyl, N-succinimidyloxyl (NHS), phenoxyl; dinitrophenoxyl; pentafluorophenoxyl, tetrafluorophenoxyl, trifluorophenoxyl, difluoro-phenoxyl, monofluoro-phenoxyl, pentachloro-phenoxyl, 1H-imidazole-1-yl, chlorophenoxyl, dichloro-phenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or carbon-oxygen), or an intermediate molecule generated with a condensation reagent for Mitsunobu reactions. The examples of condensation reagents are: EDC (N-(3-Dimethyl-aminopropyl)-N′-ethylcarbodiimide), DCC (Dicyclohexyl-carbodiimide), N,N′-Diisopropyl-carbodiimide (DIC), N-Cyclohexyl-N′-(2-morpholino-ethyl)carbodiimide metho-p-toluenesulfonate (CMC, or CME-CDI), 1,1′-Carbonyldiimi-dazole (CDI), TBTU (O-(Benzotriazol-1-yl)-N,N,N′,N′-tetra-methyluronium tetrafluoroborate), N,N,N′,N′-Tetramethyl-O-(1H-benzotriazol-1-yl)-uronium hexafluoro-phosphate (HBTU), (Benzotriazol-1-yloxy)tris-(dimethylamino)-phosphonium hexafluorophosphate (BOP), (Benzotriazol-1-yloxy)tripyrrolidinophosphonium hexafluorophosphate (PyBOP), Diethyl cyanophosphonate (DEPC), Chloro-N,N,N′,N′-tetramethylformamidiniumhexafluorophosphate, 1-[Bis(dimethylamino)methylene]-1H-1,2,3-triazolo[4,5-b]pyridinium 3-oxid hexafluorophos-phate (HATU), 1-[(Dimethylamino)(morpholino)methylene]-1H-[1,2,3]triazolo[4,5-b]pyridine-1-ium 3-oxide hexafluoro-phosphate (HDMA), 2-Chloro-1,3-dimethyl-imidazolidinium hexafluorophosphate (CIP), Chlorotripyrrolidinophosphonium hexafluorophosphate (PyCloP), Fluoro-N,N,N′,N′-bis(tetramethylene)-formamidinium hexafluorophosphate (BTFFH), N,N,N′,N′-Tetramethyl-S-(1-oxido-2-pyridyl)thiuronium hexafluorophosphate, O-(2-Oxo-1(2H)pyridyl)-N,N,N′,N′-tetramethyluronium tetrafluoroborate (TPTU), S-(1-Oxido-2-pyridyl)-N,N,N′,N′-tetramethylthiuronium tetrafluoroborate, O-[(Ethoxycarbonyl)-cyanomethylenamino]-N,N,N′,N′-tetramethyluronium hexafluorophosphate (HOTU), (1-Cyano-2-ethoxy-2-oxoethylidenamino-oxy)dimethylamino-morpholino-carbenium hexafluorophosphate (COMU), O-(Benzotriazol-1-yl)-N,N,N′,N′-bis(tetramethylene)uronium hexafluorophosphate (HBPyU), N-Benzyl-N′-cyclohexyl-carbodiimide (with, or without polymer-bound), Dipyrrolidino(N-succinimidyl-oxy)carbenium hexafluoro-phosphate (HSPyU), Chlorodipyrrolidinocarbenium hexafluoro-phosphate (PyClU), 2-Chloro-1,3-dimethylimidazolidinium tetrafluoroborate (CIB), (Benzotriazol-1-yloxy)-dipiperidino-carbenium hexafluorophosphate (HBPipU), O-(6-Chlorobenzotriazol-1-yl)-N,N,N′,N′-tetramethyluronium tetrafluoroborate (TCTU), Bromotris(dimethylamino)-phosphonium hexafluoro-phosphate (BroP), Propylphosphonic anhydride (PPACA, T3P), 2-Morpholinoethyl isocyanide (MEI), N,N,N′,N′-Tetramethyl-O—(N-succinimidyl)uronium hexafluorophosphate (HSTU), 2-Bromo-1-ethyl-pyridinium tetrafluoro-borate (BEP), O-[(Ethoxycarbonyl)cyano-methylenamino]-N,N,N′,N′-tetra-methyluronium tetrafluoroborate (TOTU), 4-(4,6-Dimethoxy-1,3,5-triazin-2-yl)-4-methylmorpholinium chloride (MMTM, DMTMM), N,N,N′,N′-Tetramethyl-O—(N-succinimidyl)uronium tetrafluoroborate (TSTU), O-(3,4-Dihydro-4-oxo-1,2,3-benzotriazin-3-yl)-N,N,N′,N′-tetramethyluronium tetrafluoro-borate (TDBTU), 1,1′-(Azodicarbonyl)-dipiperidine (ADD), Di-(4-chlorobenzyl)-azodicarboxylate (DCAD), Di-tert-butyl azodicarboxylate (DBAD), Diisopropyl azodicarboxylate (DIAD), Diethyl azodicarboxylate (DEAD). In addition, Lv can be an anhydride, formed by acid themselves or formed with other C₁˜C₈ acid anhydrides; More preferably Lv is selected from, a halide (e.g., fluoride, chloride, bromide, and iodide), maleimide, methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphrus, sulfur-nitrogen, phosphrus-nitrogen, oxygen-nitrogen, or carbon-oxygen), or one of the following structures:

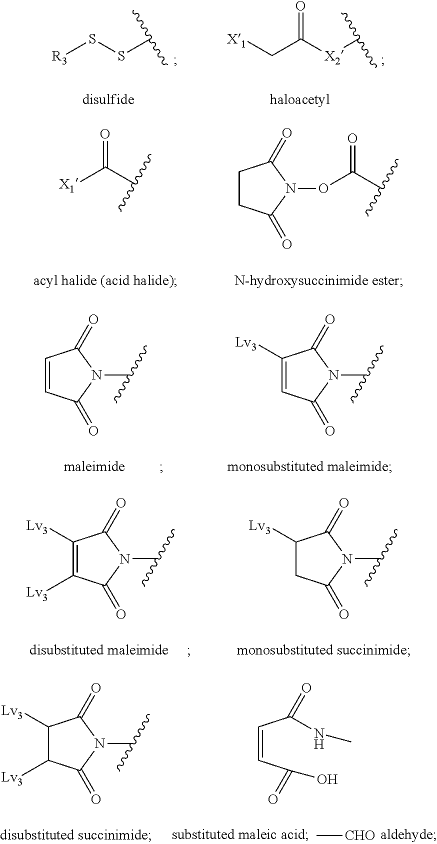

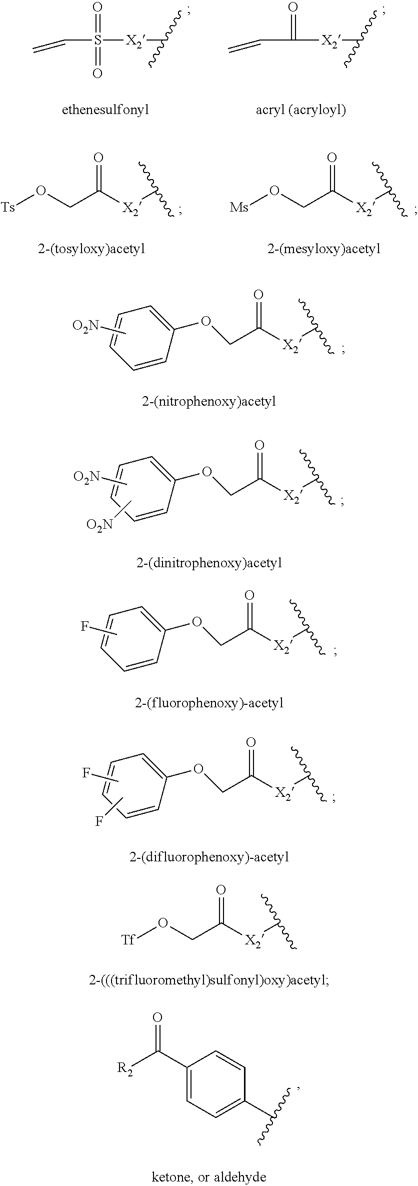

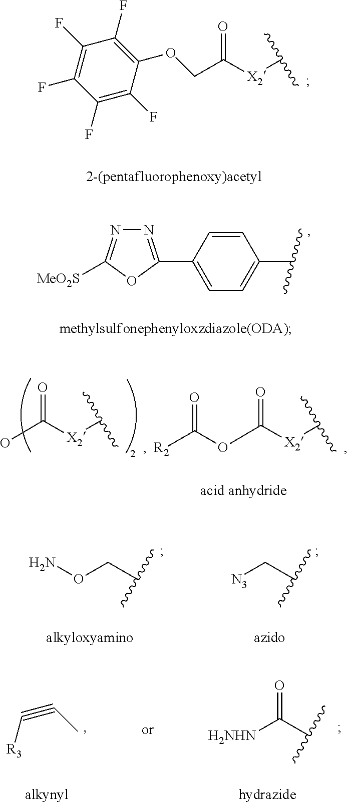

wherein X₁′ is F, Cl, Br, I or Lv₃; X₂′ is O, NH, N(R₁), or CH₂; R₃ is i H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R¹, or —COOR₁; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; trinflate; imidazole; dichlorophenol; tetrachlorophenol; 1-hydroxybenzotriazole; tosylate; mesylate; 2-ethyl-5-phenylisoxazolium-3′-sulfonate, anhydrides formed by themselves, or formed with the other anhydride, e.g. acetyl anhydride, formyl anhydride; or an intermediate molecule generated with a condensation reagent for peptide coupling reactions or for Mitsunobu reactions.

In the process of the conjugation, prior to conjugating with the Camptothecin analogs of this invention, the cell-binding molecules can be modified through attachment of a more specific peptide, a protein, or a drug, or the other functional molecules with a heterobifunctional cross linker such as with linkers of Amine-to-Nonselective (succinimidyl (NHS)-diazirine (SDA), NTS ester/Azide), Amine-to-Sulfhydryl (NHS ester/maleimide, NHS ester/pyridyldithiol, NHS esters/haloacetyl), Sulfhydryl-to-Carbohydrate (Maleimide/Hydrazide, Pyridyldithiol/Hydrazide), Hydroxyl-to-Sulfhydryl (Isocyanate/Maleimide), Amine-to-DNA (NHS ester/Psoralen), Amine-to-Carboxyl (Carbodiimide).

In the SDA linkage modification, the NIHS ester of SDA linker reacts with primary an amine group of a binding molecule backbone in pH 6˜9 buffer to form a stable amide bond upon release of NHS. Then photoactivation of the diazirine with long-wave UV light (330-370 nm) creates a reactive carbene intermediate that can react with an amine group of a more specific peptide or a protein or the other functional molecule. The order of these two steps can be different as this: an amine group of a functional molecule reacts with an SDA linker first, following by photoactive reaction of a binding molecule with long-wave UV light (330-370 nm). The SDA crosslinkers can be cleavable (with a disulfide bond inside such as SDAD linker).

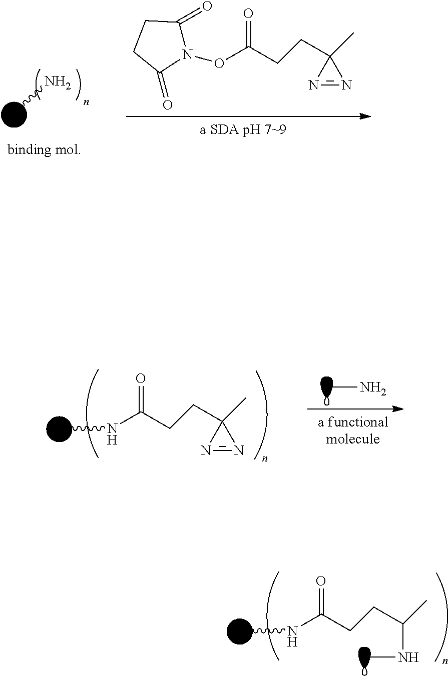

In the NHS ester/Azide linkage modification, the NIHS ester of the linker reacts with primary an amine group of a binding molecule backbone in pH 6˜9 buffer to form a stable amide. Then an alkynyl group on a more specific peptide or a protein or the other functional molecule reacts to the azido on the other side of the linker via Azide-Alkyne Huisgen Cycloaddition to form a 1,2,3-triazole linkage (click chemistry). Also, the NHS ester of the linker reacts with primary an amine group of a functional molecule in pH 6˜9 buffer to form a stable amide. Then an alkynyl group being linked on a binding molecule reacts to the azido on the other side of the linker via 5 Azide-Alkyne Huisgen Cycloaddition to form a 1,2,3-triazole linkage.

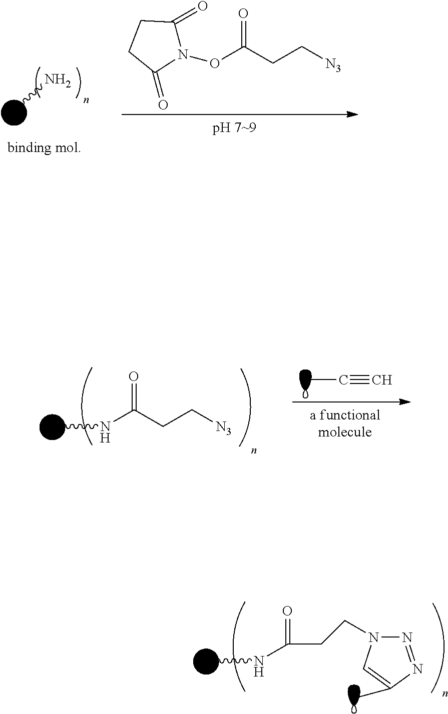

In the Anne-to-Sulfhydryl linkage modification, the NHS ester of the linker reacts with a primary amine group of a binding molecule backbone in pH 6˜9 buffer to form a stable amide bond. Then a sulfhydryl on a more specific peptide or a protein or the other functional molecule reacts to the maleimide, or pyridyldithiol, or haloacetyl on the other side of the Amine-to sulfhydryl linker at pH 4.5˜8.5 to form a thioether or a disulfide bond. The conjugation with the Amine-to-Sulfhydryl linker can be in different orders. For instance, an amine group of a functional molecule can be reacted with the linker to form an amide bond first, following by reaction with a sulfhydryl on a binding molecule. Also, a sulfhydryl group of a functional molecule can be reacted with the linker to form a thioether or a disulfide bond at pH 4.5˜7 first, following by reaction with an amine group on a binding molecule at pH 6˜9 to form an amide bond.

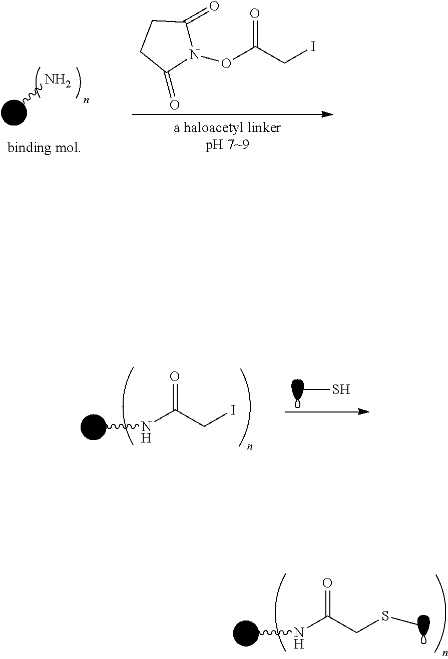

In the Sulfhydryl-to-Carbohydrate linkage modification, the sulfhydryl group of a binding molecule can be reacted with the maleimide or the pyridyldithiol on the linker to form a thioether or a disulfide bond at pH 4.5˜8 first, Then a carbonyl (aldehyde/ketone) group on a functional molecule reacts with the hydrazide to form an hydrazone bond. Also, the sulfhydryl group on a functional molecule can react with the linker to form a thioether or a disulfide bond at pH 4.5˜8 first, following by reaction with a carbohydrate, or an oxidized carbohydrate, or a carbonyl (aldehyde/ketone) group on a binding molecule form an hydrazone bond.

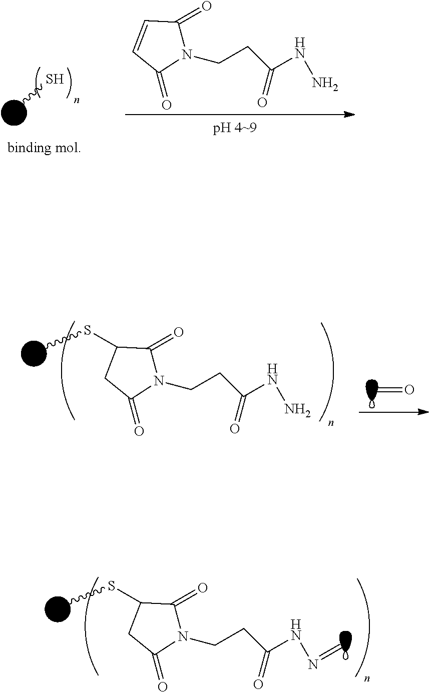

In the Hydroxyl-to-Sulfhydryl linkage modification, the sulfhydryl group of a binding molecule can be reacted with the maleimide or the pyridyldithiol on the linker to form a thioether or a disulfide bond at pH 6˜8 first, Then a hydroxy group on a functional molecule reacts with the isocyanate on the linker to form a carbamate bond at pH 8˜9. Also, the sulfhydryl group on a functional molecule can react with the linker to form a thioether or a disulfide bond at pH 6˜8 first, following by reaction with a hydroxy on a binding molecule form a carbamate bond at pH 8˜9.

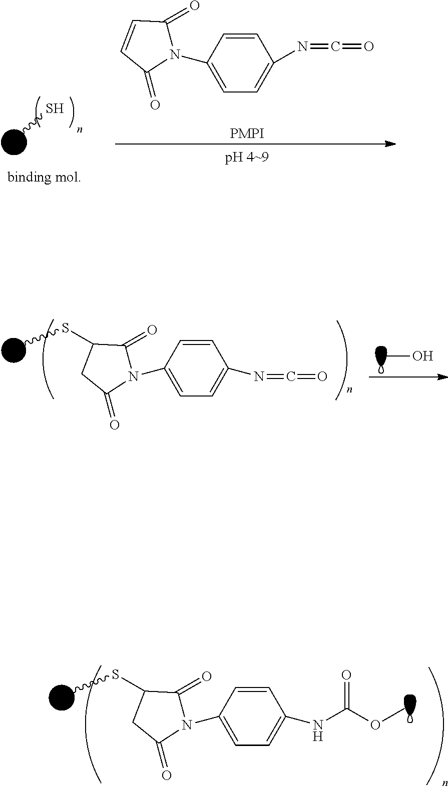

In yet another aspect of the invention, the production of antibodies used in the present invention involves in vivo or in vitro procedures or combinations thereof. Methods for producing polyclonal anti-receptor peptide antibodies are well-known in the art, such as in U.S. Pat. No. 4,493,795 (to Nestor et al). A monoclonal antibody is typically made by fusing myeloma cells with the spleen cells from a mouse that has been immunized with the desired antigen (Kohler, G.; Milstein, C. (1975). Nature 256: 495-497). The detailed procedures are described in “Antibodies—A Laboratory Manual”, Harlow and Lane, eds., Cold Spring Harbor Laboratory Press, New York (1988), which is incorporated herein by reference. Particularly monoclonal antibodies are produced by immunizing mice, rats, hamsters or any other mammal with the antigen of interest such as the intact target cell, antigens isolated from the target cell, whole virus, attenuated whole virus, and viral proteins. Splenocytes are typically fused with myeloma cells using polyethylene glycol (PEG) 6000. Fused hybrids are selected by their sensitivity to HAT (hypoxanthine-aminopterin-thymine). Hybridomas producing a monoclonal antibody useful in practicing this invention are identified by their ability to immunoreact specified receptors or inhibit receptor activity on target cells.

A monoclonal antibody used in the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques, such as using protein-A affinity chromatography; anion, cation, hydrophobic, or size exclusive chromatography (particularly by affinity for the specific antigen after Protein A, and sizing column chromatography); centrifugation, differential solubility, or by any other standard technique for the purification of proteins.

Media useful for the preparation of these compositions are both well-known in the art and commercially available and include synthetic culture media. An exemplary synthetic medium is Dulbecco's minimal essential medium (DMEM; Dulbecco et al., Virol. 8:396 (1959)) supplemented with 4.5 gm/l glucose, 20 mm glutamine, 20% fetal calf serum and with an anti-foaming agent, such as polyoxyethylene-polyoxypropylene block copolymer.

In addition, antibody-producing cell lines can also be created by techniques other than fusion, such as direct transformation of B lymphocytes with oncogenic DNA, or transfection with an oncovirus, such as Epstein-Barr virus (EBV, also called human herpesvirus 4 (HHV-4)) or Kaposi's sarcoma-associated herpesvirus (KSHV). See, U.S. Pat. Nos. 4,341,761; 4,399,121; 4,427,783; 4,444,887; 4,451,570; 4,466,917; 4,472,500; 4,491,632; 4,493,890. A monoclonal antibody may also be produced via an anti-receptor peptide or peptides containing the carboxyl terminal as described well-known in the art. See Niman et al., Proc. Natl. Acad. Sci. USA, 80: 4949-4953 (1983); Geysen et al., Proc. Natl. Acad. Sci. USA, 82: 178-182 (1985); Lei et al. Biochemistry 34(20): 6675-6688, (1995). Typically, the anti-receptor peptide or a peptide analog is used either alone or conjugated to an immunogenic carrier, as the immunogen for producing anti-receptor peptide monoclonal antibodies.

There are also a number of other well-known techniques for making monoclonal antibodies as binding molecules in this invention. Particularly useful are methods of making fully human antibodies. One method is phage display technology which can be used to select a range of human antibodies binding specifically to the antigen using methods of affinity enrichment. Phage display has been thoroughly described in the literature and the construction and screening of phage display libraries are well known in the art, see, e.g., Dente et al, Gene. 148(1):7-13 (1994); Little et al, Biotechnol Adv. 12(3):539-55 (1994); Clackson et al., Nature 352:264-628 (1991); Huse et al., Science 246:1275-1281 (1989).

Monoclonal antibodies derived by hybridoma technique from another species than human, such as mouse, can be humanized to avoid human anti-mouse antibodies when infused into humans. Among the more common methods of humanization of antibodies are complementarity-determining region grafting and resurfacing. These methods have been extensively described, see e.g. U.S. Pat. Nos. 5,859,205 and 6,797,492; Liu et al, Immunol Rev. 222:9-27 (2008); Almagro et al, Front Biosci. 1; 13: 1619-33 (2008); Lazar et al, Mol Immunol. 44(8):1986-98 (2007); Li et al, Proc. Natl. Acad. Sci. USA. 103(10):3557-62 (2006) each incorporated herein by reference. Fully human antibodies can also be prepared by immunizing transgenic mice, rabbits, monkeys, or other mammals, carrying large portions of the human immunoglobulin heavy and light chains, with an immunogen. Examples of such mice are: the Xenomouse. (Abgenix, Inc.), the HuMAb-Mouse (Medarex/BMS), the VelociMouse (Regeneron), see also U.S. Pat. Nos. 6,596,541, 6,207,418, 6,150,584, 6,111,166, 6,075,181, 5,922,545, 5,661,016, 5,545,806, 5,436,149 and 5,569,825. In human therapy, murine variable regions and human constant regions can also be fused to construct called “chimeric antibodies” that are considerably less immunogenic in man than murine mAbs (Kipriyanov et al, Mol Biotechnol. 26:39-60 (2004); Houdebine, Curr Opin Biotechnol. 13:625-9 (2002) each incorporated herein by reference). In addition, site-directed mutagenesis in the variable region of an antibody can result in an antibody with higher affinity and specificity for its antigen (Brannigan et al, Nat Rev Mol Cell Biol. 3:964-70, (2002)); Adams et al, J Immunol Methods. 231:249-60 (1999)) and exchanging constant regions of a mAb can improve its ability to mediate effector functions of binding and cytotoxicity.

Antibodies immunospecific for a malignant cell antigen can also be obtained commercially or produced by any method known to one of skill in the art such as, e.g., chemical synthesis or recombinant expression techniques. The nucleotide sequence encoding antibodies immunospecific for a malignant cell antigen can be obtained commercially, e.g., from the GenBank database or a database like it, the literature publications, or by routine cloning and sequencing.

Apart from an antibody, a peptide or protein that bind/block/target or in some other way interact with the epitopes or corresponding receptors on a targeted cell can be used as a binding molecule. These peptides or proteins could be any random peptide or proteins that have an affinity for the epitopes or corresponding receptors and they don't necessarily have to be of the immunoglobulin family. These peptides can be isolated by similar techniques as for phage display antibodies (Szardenings, J Recept Signal Transduct Res. 2003; 23(4):307-49). The use of peptides from such random peptide libraries can be similar to antibodies and antibody fragments. The binding molecules of peptides or proteins may be conjugated on or linked to a large molecules or materials, such as, but is not limited, an albumin, a polymer, a liposome, a nano particle, as long as such attachment permits the peptide or protein to retain its antigen binding specificity.

Examples of antibodies used for conjugation of Camptothecin analogs in this prevention for treating cancer, autoimmune disease, and infectious disease include, but are not limited to, 3F8 (anti-GD2), Abagovomab (anti CA-125), Abciximab (anti CD41 (integrin alpha-IIb), Adalimumab (anti-TNF-α), Adecatumumab (anti-EpCAM, CD326), Afelimomab (anti-TNF-α); Afutuzumab (anti-CD20), Alacizumab pegol (anti-VEGFR2), ALD518 (anti-IL-6), Alemtuzumab (Campath, MabCampath, anti-CD52), Altumomab (anti-CEA), Anatumomab (anti-TAG-72), Anrukinzumab (IMA-638, anti-IL-13), Apolizumab (anti-HLA-DR), Arcitumomab (anti-CEA), Aselizumab (anti-L-selectin (CD62L), Atlizumab (tocilizumab, Actemra, RoActemra, anti-IL-6 receptor), Atorolimumab (anti-Rhesus factor), Bapineuzumab (anti-beta amyloid), Basiliximab (Simulect, antiCD25 (a chain of IL-2 receptor), Bavituximab (anti-phosphatidylserine), Bectumomab (LymphoScan, anti-CD22), Belimumab (Benlysta, LymphoStat-B, anti-BAFF), Benralizumab (anti-CD125), Bertilimumab (anti-CCL11 (eotaxin-1)), Besilesomab (Scintimun, anti-CEA-related antigen), Bevacizumab (Avastin, anti-VEGF-A), Biciromab (FibriScint, anti-fibrin II beta chain), Bivatuzumab (anti-CD44 v6), Blinatumomab (BiTE, anti-CD19), Brentuximab (cAC10, anti-CD30 TNFRSF8), Briakinumab (anti-IL-12, IL-23) Canakinumab (Ilaris, anti-IL-1), Cantuzumab (C242, anti-CanAg), Capromab, Catumaxomab (Removab, anti-EpCAM, anti-CD3), CC49 (anti-TAG-72), Cedelizumab (anti-CD4), Certolizumab pegol (Cimzia anti-TNF-α), Cetuximab (Erbitux, IMC-C225, anti-EGFR), Citatuzumab bogatox (anti-EpCAM), Cixutumumab (anti-IGF-1), Clenoliximab (anti-CD4), Clivatuzumab (anti-MUC1), Conatumumab (anti-TRAIL-R2), CR6261 (anti-Influenza A hemagglutinin), Dacetuzumab (anti-CD40), Daclizumab (Zenapax, anti-CD25 (α chain of IL-2 receptor)), Daratumumab (anti-CD38 (cyclic ADP ribose hydrolase), Denosumab (Prolia, anti-RANKL), Detumomab (anti-B-lymphoma cell), Dorlimomab, Dorlixizumab, Ecromeximab (anti-GD3 ganglioside), Eculizumab (Soliris, anti-C5), Edobacomab (anti-endotoxin), Edrecolomab (Panorex, MAb17-1A, anti-EpCAM), Efalizumab (Raptiva, anti-LFA-1 (CD11a), Efungumab (Mycograb, anti-Hsp90), Elotuzumab (anti-SLAMF7), Elsilimomab (anti-IL-6), Enlimomab pegol (anti-ICAM-1 (CD54)), Epitumomab (anti-episialin), Epratuzumab (anti-CD22), Erlizumab (anti-ITGB2 (CD18)), Ertumaxomab (Rexomun, anti-HER2/neu, CD3), Etaracizumab (Abegrin, anti-integrin (aᵥβ₃), Exbivirumab (anti-hepatitis B surface antigen), Fanolesomab (NeutroSpec, anti-CD15), Faralimomab (anti-interferon receptor), Farletuzumab (anti-folate receptor 1), Felvizumab (anti-respiratory syncytial virus), Fezakinumab (anti-IL-22), Figitumumab (anti-IGF-1 receptor), Fontolizumab (anti-IFN-γ), Foravirumab (anti-rabies virus glycoprotein), Fresolimumab (anti-TGF-β), Galiximab (anti-CD80), Gantenerumab (anti-beta amyloid), Gavilimomab (anti-CD147 (basigin)), Gemtuzumab (anti-CD33), Girentuximab (anti-carbonic anhydrase 9), Glembatumumab (CR011, anti-GPNMB), Golimumab (Simponi, anti-TNF-α), Gomiliximab (anti-CD23 (IgE receptor)), anti-HLA-DR antibody, Ibalizumab (anti-CD4), Ibritumomab (anti-CD20), Igovomab (Indimacis-125, anti-CA-125), Imciromab (Myoscint, anti-cardiac myosin), Infliximab (Remicade, anti-TNF-α), Intetumumab (anti-CD51), Inolimomab (anti-CD25 (α chain of IL-2 receptor)), Inotuzumab (anti-CD22), Ipilimumab (anti-CD152), Iratumumab (anti-CD30 (TNFRSF8)), Keliximab (anti-CD4), Labetuzumab (CEA-Cide, anti-CEA), Lebrikizumab (anti-IL-13), Lemalesomab (anti-NCA-90 (granulocyte antigen)), Lerdelimumab (anti-TGF beta 2), Lexatumumab (anti-TRAIL-R2), Libivirumab (anti-hepatitis B surface antigen), Lintuzumab (anti-CD33), Lucatumumab (anti-CD40), Lumiliximab (anti-CD23 (IgE receptor), Mapatumumab (anti-TRAIL-R1), Maslimomab (anti-T-cell receptor), Matuzumab (anti-EGFR), Mepolizumab (Bosatria, anti-IL-5), Metelimumab (anti-TGF beta 1), Milatuzumab (anti-CD74), Minretumomab (anti-TAG-72), Mitumomab (BEC-2, anti-GD3 ganglioside), Morolimumab (anti-Rhesus factor), Motavizumab (Numax, anti-respiratory syncytial virus), Muromonab-CD3 (Orthoclone OKT3, anti-CD3), Nacolomab (anti-C242), Naptumomab (anti-5T4), Natalizumab (Tysabri, anti-integrin α₄), Nebacumab (anti-endotoxin), Necitumumab (anti-EGFR), Nerelimomab (anti-TNF-α), Nimotuzumab (Theracim, Theraloc, anti-EGFR), Nofetumomab, Ocrelizumab (anti-CD20), Odulimomab (Afolimomab, anti-LFA-1 (CD11a)), Ofatumumab (Arzerra, anti-CD20), Olaratumab (anti-PDGF-Rα), Omalizumab (Xolair, anti-IgE Fc region), Oportuzumab (anti-EpCAM), Oregovomab (OvaRex, anti-CA-125), Otelixizumab (anti-CD3), Pagibaximab (anti-lipoteichoic acid), Palivizumab (Synagis, Abbosynagis, anti-respiratory syncytial virus), Panitumumab (Vectibix, ABX-EGF, anti-EGFR), Panobacumab (anti-Pseudomonas aeruginosa), Pascolizumab (anti-IL-4), Pemtumomab (Theragyn, anti-MUC1), Pertuzumab (Omnitarg, 2C4, anti-HER2/neu), Pexelizumab (anti-C5), Pintumomab (anti-adenocarcinoma antigen), Priliximab (anti-CD4), Pritumumab (anti-vimentin), PRO 140 (anti-CCR5), Racotumomab (1E10, anti-(N-glycolylneuraminic acid (NeuGc, NGNA)-gangliosides GM3)), Rafivirumab (anti-rabies virus glycoprotein), Ramucirumab (anti-VEGFR2), Ranibizumab (Lucentis, anti-VEGF-A), Raxibacumab (anti-anthrax toxin, protective antigen), Regavirumab (anti-cytomegalovirus glycoprotein B), Reslizumab (anti-IL-5), Rilotumumab (anti-HGF), Rituximab (MabThera, Rituxanmab, anti-CD20), Robatumumab (anti-IGF-1 receptor), Rontalizumab (anti-IFN-α), Rovelizumab (LeukArrest, anti-CD11, CD18), Ruplizumab (Antova, anti-CD154 (CD40L)), Satumomab (anti-TAG-72), Sevirumab (anti-cytomegalovirus), Sibrotuzumab (anti-FAP), Sifalimumab (anti-IFN-α), Siltuximab (anti-IL-6), Siplizumab (anti-CD2), (Smart) MI95 (anti-CD33), Solanezumab (anti-beta amyloid), Sonepcizumab (anti-sphingosine-1-phosphate), Sontuzumab (anti-episialin), Stamulumab (anti-myostatin), Sulesomab (LeukoScan, (anti-NCA-90 (granulocyte antigen), Tacatuzumab (anti-alpha-fetoprotein), Tadocizumab (anti-integrin α_{IIb}β₃), Talizumab (anti-IgE), Tanezumab (anti-NGF), Taplitumomab (anti-CD19), Tefibazumab (Aurexis, (anti-clumping factor A), Telimomab, Tenatumomab (anti-tenascin C), Teneliximab (anti-CD40), Teplizumab (anti-CD3), TGN1412 (anti-CD28), Ticilimumab (Tremelimumab, (anti-CTLA-4), Tigatuzumab (anti-TRAIL-R2), TNX-650 (anti-IL-13), Tocilizumab (Atlizumab, Actemra, RoActemra, (anti-IL-6 receptor), Toralizumab (anti-CD154 (CD40L)), Tositumomab (anti-CD20), Trastuzumab (Herceptin, (anti-HER2/neu), Tremelimumab (anti-CTLA-4), Tucotuzumab celmoleukin (anti-EpCAM), Tuvirumab (anti-hepatitis B virus), Urtoxazumab (anti-Escherichia coli), Ustekinumab (Stelara, anti-IL-12, IL-23), Vapaliximab (anti-AOC3 (VAP-1)), Vedolizumab, (anti-integrin a407), Veltuzumab (anti-CD20), Vepalimomab (anti-AOC3 (VAP-1), Visilizumab (Nuvion, anti-CD3), Vitaxin (anti-vascular integrin avb3), Volociximab (anti-integrin α₅β₁), Votumumab (HumaSPECT, anti-tumor antigen CTAA16.88), Zalutumumab (HuMax-EGFr, (anti-EGFR), Zanolimumab (HuMax-CD4, anti-CD4), Ziralimumab (anti-CD147 (basigin)), Zolimomab (anti-CD5), Etanercept (Enbrel®), Alefacept (Amevive®), Abatacept (Orencia®), Rilonacept (Arcalyst), 14F7 [anti-IRP-2 (Iron Regulatory Protein 2)], 14G2a (anti-GD2 ganglioside, from Nat. Cancer Inst. for melanoma and solid tumors), J591 (anti-PSMA, Weill Cornell Medical School for prostate cancers), 225.28S [anti-HMW-MAA (High molecular weight-melanoma-associated antigen), Sorin Radiofarmaci S.R.L. (Milan, Italy) for melanoma], COL-1 (anti-CEACAM3, CGM1, from Nat. Cancer Inst. USA for colorectal and gastric cancers), CYT-356 (Oncoltad®, for prostate cancers), HNK20 (OraVax Inc. for respiratory syncytial virus), ImmuRAIT (from Immunomedics for NHL), Lym-1 (anti-HLA-DR10, Peregrine Pharm. for Cancers), MAK-195F [anti-TNF (tumor necrosis factor; TNFA, TNF-alpha; TNFSF2), from Abbott/Knoll for Sepsis toxic shock], MEDI-500 [T10B9, anti-CD3, TRαβ (T cell receptor alpha/beta), complex, from MedImmune Inc for Graft-versus-host disease], RING SCAN [anti-TAG 72 (tumour associated glycoprotein 72), from Neoprobe Corp. for Breast, Colon and Rectal cancers], Avicidin (anti-EPCAM (epithelial cell adhesion molecule), anti-TACSTD1 (Tumor-associated calcium signal transducer 1), anti-GA733-2 (gastrointestinal tumor-associated protein 2), anti-EGP-2 (epithelial glycoprotein 2); anti-KSA; KS1/4 antigen; M4S; tumor antigen 17-1A; CD326, from NeoRx Corp. for Colon, Ovarian, Prostate cancers and NHL]; anti-Trop-2-humanized antibody hRS7, LymphoCide (Immunomedics, NJ), Smart ID10 (Protein Design Labs), Oncolym (Techniclone Inc, CA), Allomune (BioTransplant, CA), anti-VEGF (Genentech, CA); CEAcide (Immunomedics, NJ), IMC-1C11 (ImClone Systems) and Cetuximab (ImClone).

Other antibodies as binding ligands include, but are not limited to, are antibodies against the following antigens: Aminopeptidase N (CD13), Annexin A1, B7-H3 (CD276, various cancers), CA125 (ovarian), CA15-3 (carcinomas), CA19-9 (carcinomas), L6 (carcinomas), Lewis Y (carcinomas), Lewis X (carcinomas), alpha fetoprotein (carcinomas), CA242 (colorectal), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (Metastatic melanoma, sarcoma), CD52 (leukemia), CD56 (small cell lung cancers, ovarian cancer, Merkel cell carcinoma, and the liquid tumor, multiple myeloma), CD66e (cancers), CD70 (metastatic renal cell carcinoma and non-Hodgkin lymphoma), CD74 (multiple myeloma), CD80 (lymphoma), CD98 (cancers), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (carcinoembryonic antigen; CEA, CD66e) (breast, colorectal and lung cancers), DLL4 (A-like-4), EGFR (Epidermal Growth Factor Receptor, various cancers), CTLA4 (melanoma), CXCR4 (CD184, Heme-oncology, solid tumors), Endoglin (CD105, solid tumors), EPCAM (epithelial cell adhesion molecule, bladder, head, neck, colon, NHL prostate, and ovarian cancers), ERBB2 (Epidermal Growth Factor Receptor 2; lung, breast, prostate cancers), FCGR1 (autoimmune diseases), FOLR (folate receptor, ovarian cancers), GD2 ganglioside (cancers), G-28 (a cell surface antigen glyvolipid, melanoma), GD3 idiotype (cancers), Heat shock proteins (cancers), HER1 (lung, stomach cancers), HER2 (breast, lung and ovarian cancers), HLA-DR10 (NHL), HLA-DRB (NHL, B cell leukemia), human chorionic gonadotropin (carcinoma), IGF1R (insulin-like growth factor 1 receptor, solid tumors, blood cancers), IL-2 receptor (interleukin 2 receptor, T-cell leukemia and lymphomas), IL-6R (interleukin 6 receptor, multiple myeloma, RA, Castleman's disease, IL6 dependent tumors), Integrins (αvβ3, α5β1, α6β4, α11β3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 or MUC1-KLH (breast, ovarian, cervix, bronchus and gastrointestinal cancer), MUC16 (CA125) (Ovarian cancers), CEA (colorectal), gp100 (melanoma), MART1 (melanoma), MPG (melanoma), MS4A1 (membrane-spanning 4-domains subfamily A, small cell lung cancers, NHL), Nucleolin, Neu oncogene product (carcinomas), P21 (carcinomas), Paratope of anti-(N-glycolylneuraminic acid, Breast, Melanoma cancers), PLAP-like testicular alkaline phosphatase (ovarian, testicular cancers), PSMA (prostate tumors), PSA (prostate), ROBO4, TAG 72 (tumour associated glycoprotein 72, AML, gastric, colorectal, ovarian cancers), T cell transmembrane protein (cancers), Tie (CD202b), TNFRSF10B (tumor necrosis factor receptor superfamily member 10B, cancers), TNFRSF13B (tumor necrosis factor receptor superfamily member 13B, multiple myeloma, NHL, other cancers, RA and SLE), TPBG (trophoblast glycoprotein, Renal cell carcinoma), TRAIL-R1 (Tumor necrosis apoprosis Inducing ligand Receptor 1, lymphoma, NHL, colorectal, lung cancers), VCAM-1 (CD106, Melanoma), VEGF, VEGF-A, VEGF-2 (CD309) (various cancers). Some other tumor associated antigens recognized by antibodies have been reviewed (Gerber, et al, mAbs 1:3, 247-253 (2009); Novellino et al, Cancer Immunol Immunother. 54(3), 187-207 (2005). Franke, et al, Cancer Biother Radiopharm. 2000, 15, 459-76). Examples of these antigens that antibodies against are: Many other Cluster of Differentiations (CD4, CD5, CD6, CD7, CD8, CD9, CD10, CD11a, CD11b, CD11c, CD12w, CD14, CD15, CD16, CDw17, CD18, CD21, CD23, CD24, CD25, CD26, CD27, CD28, CD29, CD31, CD32, CD34, CD35, CD36, CD37, CD41, CD42, CD43, CD44, CD45, CD46, CD47, CD48, CD49b, CD49c, CD53, CD54, CD55, CD58, CD59, CD61, CD62E, CD62L, CD62P, CD63, CD68, CD69, CD71, CD72, CD79, CD81, CD82, CD83, CD86, CD87, CD88, CD89, CD90, CD91, CD95, CD96, CD100, CD103, CD105, CD106, CD109, CD117, CD120, CD127, CD133, CD134, CD135, CD138, CD141, CD142, CD143, CD144, CD147, CD151, CD152, CD154, CD156, CD158, CD163, CD166, CD168, CD184, CDw186, CD195, CD202 (a, b), CD209, CD235a, CD271, CD303, CD304), Annexin A1, Nucleolin, Endoglin (CD105), ROBO4, Amino-peptidase N, Δ-like-4 (DLL4), VEGFR-2 (CD309), CXCR4 9CD184), Tie2, B7-H3, WT1, MUC1, LMP2, HPV E6 E7, EGFRvIII, HER-2/neu, Idiotype, MAGE A3, p₅₃ nonmutant, NY-ESO-1, GD2, CEA, MelanA/MART1, Ras mutant, gp100, p53 mutant, Proteinase3 (PR1), ber-abl, Tyrosinase, Survivin, hTERT, Sarcoma translocation breakpoints, EphA2, PAP, ML-IAP, AFP, EpCAM, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-0, MAD-CT-2, Fos-related antigen 1.

In another specific embodiment, the Camptothecin analog-binding molecule conjugates of the invention are used in accordance with the compositions and methods of the invention for the treatment of cancers. The cancers include, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metastatic Squamous Neck Cancer with Occult Primary, Multiple Myeloma, and Other Plasma Cell Neoplasms, Mycosis Fungoides, Myelodysplastic Syndrome, Myeloproliferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor

In another specific embodiment, the Camptothecin analog-binding molecule conjugates of the invention are used in accordance with the compositions and methods of the invention for the treatment or prevention of an autoimmune disease. The autoimmune diseases include, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaff's encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barre syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogammaglobulinemia, Idiopathic Inflammatory Demyelinating Diseases, Idiopathic pulmonary fibrosis, Idiopathic thrombocytopenic purpura (See Autoimmune thrombocytopenic purpura), IgA nephropathy (Also Berger's disease), Inclusion body myositis, Inflammatory demyelinating polyneuropathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, Meniere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjögren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome

In another specific embodiment, a binding molecule used for the conjugate for the treatment or prevention of an autoimmune disease includes, but are not limited to, anti-elastin antibody; Abys against epithelial cells antibody; Anti-Basement Membrane Collagen Type IV Protein antibody; Anti-Nuclear Antibody; Anti ds DNA; Anti ss DNA, Anti Cardiolipin Antibody IgM, IgG; anti-celiac antibody; Anti Phospholipid Antibody IgK, IgG; Anti SM Antibody; Anti Mitochondrial Antibody; Thyroid Antibody; Microsomal Antibody, T-cells antibody; Thyroglobulin Antibody, Anti SCL-70; Anti-Jo; Anti-U.sub.1RNP; Anti-La/SSB; Anti SSA; Anti SSB; Anti Perital Cells Antibody; Anti Histones; Anti RNP; C-ANCA; P-ANCA; Anti centromere; Anti-Fibrillarin, and Anti GBM Antibody, Anti-ganglioside antibody; Anti-Desmogein 3 antibody; Anti-p62 antibody; Anti-sp100 antibody; Anti-Mitochondrial (M2) antibody; Rheumatoid factor antibody; Anti-MCV antibody; Anti-topoisomerase antibody; Anti-neutrophil cytoplasmic (cANCA) antibody.

In certain preferred embodiments, the binding molecule for the conjugate in the present invention, can bind to both a receptor or a receptor complex expressed on an activated lymphocyte which is associated with an autoimmune disease. The receptor or receptor complex can comprise an immunoglobulin gene superfamily member (e.g. CD2, CD3, CD4, CD8, CD19, CD22, CD28, CD79, CD90, CD152/CTLA-4, PD-1, or ICOS), a TNF receptor superfamily member (e.g. CD27, CD40, CD95/Fas, CD134/OX40, CD137/4-1BB, INF-R1, TNFR-2, RANK, TACI, BCMA, osteoprotegerin, Apo2/TRAIL-R1, TRAIL-R2, TRAIL-R3, TRAIL-R4, and APO-3), an integrin, a cytokine receptor, a chemokine receptor, a major histocompatibility protein, a lectin (C-type, S-type, or I-type), or a complement control protein.

In another specific embodiment, useful binding ligands that are immunospecific for a viral or a microbial antigen are humanized or human monoclonal antibodies. As used herein, the term “viral antigen” includes, but is not limited to, any viral peptide, polypeptide protein (e.g. HIV gp120, HIV nef, RSV F glycoprotein, influenza virus neuramimidase, influenza virus hemagglutinin, HTLV tax, herpes simplex virus glycoprotein (e.g. gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., a bacterial, fungi, pathogenic protozoa, or yeast polypeptide including, e.g., LPS and capsular polysaccharide 5/8) that is capable of eliciting an immune response. Examples of antibodies available 1 for the viral or microbial infection include, but are not limited to, Palivizumab which is a humanized anti-respiratory syncytial virus monoclonal antibody for the treatment of RSV infection; PR0542 which is a CD4 fusion antibody for the treatment of HIV infection; Ostavir which is a human antibody for the treatment of hepatitis B virus; PROTVIR which is a humanized IgG.sub.1 antibody for the treatment of cytomegalovirus; and anti-LPS antibodies.

The binding molecules-Camptothecin analog conjugates of this invention can be used in the treatment of infectious diseases. These infectious diseases include, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcanobacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioidomycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome, Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylon infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsialpox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuberculosis infection, Yersiniosis, Yellow fever, Zygomycosis.

The binding molecules, proffered antibodies described in this patent that are against pathogenic strains include, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4)—Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli O157:H₇, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomyxoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthoramycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

Other antibodies as a binding ligand in this invention for treatment of viral disease include, but are not limited to, antibodies against antigens of pathogenic viruses, including as examples and not by limitation: Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytial virus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the conjugate of the invention together with a pharmaceutically acceptable carrier for treatment of cancer and autoimmune disorders. The method for treatment of cancer and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 15 minutes to about 48 hours at about 37° C. The exact conditions of concentration and time of incubation (=dose) are readily determined by the skilled clinicians. After incubation the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

A stable conjugate should also “retains its biological activity” in a pharmaceutical formulation, if the biological activity of the conjugate at a given time, e. g. 12 month, within about 20%, preferably about 10% (within the errors of the assay) of the biological activity exhibited at the time the pharmaceutical formulation was prepared as determined in an antigen binding assay, and/or in vitro, cytotoxic assay, for example.

For clinical in vivo use, the conjugate of the invention will be supplied as solutions or as a lyophilized solid that can be dissolved in sterile water for injection. Examples of suitable protocols of conjugate administration are as follows. Conjugates are given daily, weekly, biweekly, triweekly, once every four weeks or monthly for 8-54 weeks as an i.v. bolus. Bolus doses are given in 50 to 1000 ml of normal saline to which human serum albumin (e.g. 0.5 to 1 mL of a concentrated solution of human serum albumin, 100 mg/mL) can optionally be added. Dosages will be about 50 μg to 20 mg/kg of body weight per week, i.v. (range of 10 μg to 200 mg/kg per injection). 4˜54 weeks after treatment, the patient may receive a second course of treatment. Specific clinical protocols with regard to route of administration, excipients, diluents, dosages, times, etc., can be determined by the skilled clinicians.

Examples of medical conditions that can be treated according to the in vivo or ex vivo methods of killing selected cell populations include malignancy of any types of cancer, autoimmune diseases, graft rejections, and infections (viral, bacterial or parasite).

The amount of a conjugate which is required to achieve the desired biological effect, will vary depending upon a number of factors, including the chemical characteristics, the potency, and the bioavailability of the conjugates, the type of disease, the species to which the patient belongs, the diseased state of the patient, the route of administration, all factors which dictate the required dose amounts, delivery and regimen to be administered.

In general terms, the conjugates via the linkers of this invention may be provided in an aqueous physiological buffer solution containing 0.1 to 10% w/v conjugates for parenteral administration. Typical dose ranges are from 1 μg/kg to 0.1 g/kg of body weight daily; weekly, biweekly, triweekly, or monthly, a preferred dose range is from 0.01 mg/kg to 20 mg/kg of body weight weekly, biweekly, triweekly, or monthly, an equivalent dose in a human. The preferred dosage of drug to be administered is likely to depend on such variables as the type and extent of progression of the disease or disorder, the overall health status of the particular patient, the relative biological efficacy of the compound selected, the formulation of the compound, the route of administration (intravenous, intramuscular, or other), the pharmacokinetic properties of the conjugates by the chosen delivery route, and the speed (bolus or continuous infusion) and schedule of administrations (number of repetitions in a given period of time).

The conjugates of the present invention are also capable of being administered in unit dose forms, wherein the term “unit dose” means a single dose which is capable of being administered to a patient, and which can be readily handled and packaged, remaining as a physically and chemically stable unit dose comprising either the active conjugate itself, or as a pharmaceutically acceptable composition, as described hereinafter. As such, typical total daily/weekly/biweekly/monthly dose ranges are from 0.01 to 100 mg/kg of body weight. By way of general guidance, unit doses for humans range from 1 mg to 3000 mg per day, or per week, per two weeks (biweekly), triweekly, or per month. Preferably the unit dose range is from 1 to 500 mg administered one to four times a month and even more preferably from 1 mg to 100 mg, once a week, or once biweekly, or once a triweekly. Conjugates provided herein can be formulated into pharmaceutical compositions by admixture with one or more pharmaceutically acceptable excipients. Such unit dose compositions may be prepared for use by oral administration, particularly in the form of tablets, simple capsules or soft gel capsules; or intranasally, particularly in the form of powders, nasal drops, or aerosols; or dermally, for example, topically in ointments, creams, lotions, gels or sprays, or via trans-dermal patches. The compositions may conveniently be administered in unit dosage form and may be prepared by any of the methods well known in the pharmaceutical art, for example, as described in Remington: The Science and Practice of Pharmacy, 21 ed.; Lippincott Williams & Wilkins: Philadelphia, Pa., 2005.

Preferred formulations include pharmaceutical compositions in which a compound of the present invention is formulated for oral or parenteral administration. For oral administration, tablets, pills, powders, capsules, troches and the like can contain one or more of any of the following ingredients, or compounds of a similar nature: a binder such as microcrystalline cellulose, or gum tragacanthin; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal. Preferred tablets contain lactose, cornstarch, magnesium silicate, croscarmellose sodium, povidone, magnesium stearate, or talc in any combination.

Liquid preparations for parenteral administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, vegetable oils such as olive oil, and organic esters such as ethyl oleate. Aqueous carriers include mixtures of alcohols and water, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like. Other potentially useful parenteral delivery systems for these active compounds include ethylene-vinyl acetate copolymer particles, osmotic pumps, implantable infusion systems, and liposomes.

Alternative modes of administration include formulations for inhalation, which include such means as dry powder, aerosol, or drops. They may be aqueous solutions containing, for example, polyoxyethylene-9-lauryl ether, glycocholate and deoxycholate, or oily solutions for administration in the form of nasal drops, or as a gel to be applied intranasally. Formulations for buccal administration include, for example, lozenges or pastilles and may also include a flavored base, such as sucrose or acacia, and other excipients such as glycocholate. Formulations suitable for rectal administration are preferably presented as unit-dose suppositories, with a solid based carrier, such as cocoa butter, and may include a salicylate. Formulations for topical application to the skin preferably take the form of an ointment, cream, lotion, paste, gel, spray, aerosol, or oil. Carriers which can be used include petroleum jelly, lanolin, polyethylene glycols, alcohols, or their combinations. Formulations suitable for transdermal administration can be presented as discrete patches and can be lipophilic emulsions or buffered, aqueous solutions, dissolved and/or dispersed in a polymer or an adhesive.

In a specific embodiment, a conjugate of the invention is administered concurrently with the other known or will be known therapeutic agents such as the chemotherapeutic agent, the radiation therapy, immunotherapy agents, autoimmune disorder agents, anti-infectious agents or the other antibody-drug conjugates, resulting in a synergistic effect. In another specific embodiment, the synergistic drugs or radiation therapy are administered prior or subsequent to administration of a conjugate, in one aspect at least an hour, 12 hours, a day, a week, biweeks, triweeks, a month, in further aspects several months, prior or subsequent to administration of a conjugate of the invention.

In other embodiments, the synergistic drugs include, but not limited to:

1). Chemotherapeutic agents: a). Alkylating agents: such as Nitrogen mustards: chlorambucil, chlomaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 (including its adozelesin, carzelesin and bizelesin synthetic analogues); Duocarmycin (including the synthetic analogues, KW-2189 and CBI-TMI); Benzodiazepine dimers (e.g., dimmers of pyrrolobenzodiazepine (PBD) or tomaymycin, indolinobenzodiazepines, imidazobenzothiadiazepines, or oxazolidino-benzodiazepines); Nitrosoureas: (carmustine, lomustine, chlorozotocin, fotemustine, nimustine, ranimustine); Alkylsulphonates: (busulfan, treosulfan, improsulfan and piposulfan); Triazenes: (dacarbazine); Platinum containing compounds: (carboplatin, cisplatin, oxaliplatin); aziridines, such as benzodopa, carboquone, meturedopa, and uredopa; ethylenimines and methylamelamines including altretamine, triethylenemel-amine, trietylenephosphoramide, triethylenethio-phosphaoramide and trimethylolomel-amine]; b). Plant Alkaloids: such as Vinca alkaloids: (vincristine, vinblastine, vindesine, vinorelbine, navelbin); Taxoids: (paclitaxel, docetaxol) and their analogs, Maytansinoids (DM1, DM2, DM3, DM4, maytansine and ansamitocins) and their analogs, cryptophycins (particularly cryptophycin 1 and cryptophycin 8); epothilones, eleutherobin, discodermo-lide, bryostatins, dolostatins, auristatins, amatoxins, cephalostatins; pancratistatin; a sarcodictyin; spongistatin; c). DNA Topoisomerase Inhibitors: such as [Epipodophyllins: (9-aminocamptothecin, camptothecin, crisnatol, daunomycin, etoposide, etoposide phosphate, irinotecan, mitoxantrone, novantrone, retinoic acids (retinols), teniposide, topotecan, 9-nitrocamptothecin (RFS 2000)); mitomycins: (mitomycin C)]; d). Anti-metabolites: such as {[Anti-folate: DHFR inhibitors: (methotrexate, trimetrexate, denopterin, pteropterin, aminopterin (4-aminopteroic acid) or the other folic acid analogues); IMP dehydrogenase Inhibitors: (mycophenolic acid, tiazofurin, ribavirin, EICAR); Ribonucleotide reductase Inhibitors: (hydroxyurea, deferoxamine)]; [Pyrimidine analogs: Uracil analogs: (ancitabine, azacitidine, 6-azauridine, capecitabine (Xeloda), carmofur, cytarabine, dideoxyuridine, doxifluridine, enocitabine, 5-Fluorouracil, floxuridine, ratitrexed (Tomudex)); Cytosine analogs: (cytarabine, cytosine arabinoside, fludarabine); Purine analogs: (azathioprine, fludarabine, mercaptopurine, thiamiprine, thioguanine)]; folic acid replenisher, such as frolinic acid}; e). Hormonal therapies: such as {Receptor antagonists: [Anti-estrogen: (megestrol, raloxifene, tamoxifen); LHRH agonists: (goserelin, leuprolide acetate); Anti-androgens: (bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (CB 1093, EB 1089 KH 1060, cholecalciferol, ergocalciferol); Photodynamic therapies: (verteporfin, phthalocyanine, photosensitizer Pc4, demethoxy-hypocrellin A); Cytokines: (Interferon-alpha, Interferon-gamma, tumor necrosis factor (TNFs), human proteins containing a TNF domain)]}; f). Kinase inhibitors, such as BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib; g). A poly (ADP-ribose) polymerase (PARP) inhibitors, such as olaparib, niraparib, iniparib, talazoparib, veliparib, veliparib, CEP 9722 (Cephalon's), E7016 (Eisai's), BGB-290 (BeiGene's), 3-aminobenzamide.

h). antibiotics, such as the enediyne antibiotics (e.g. calicheamicins, especially calicheamicin γ1, δ1, α1 and β1, see, e.g., J. Med. Chem., 39 (11), 2103-2117 (1996), Angew Chem Intl. Ed. Engl. 33:183-186 (1994); dynemicin, including dynemicin A and deoxydynemicin; esperamicin, kedarcidin, C-1027, maduropeptin, as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; i). Others: such as Polyketides (acetogenins), especially bullatacin and bullatacinone; gemcitabine, epoxomicins (e. g. carfilzomib), bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors (such as Lovastatin), Dopaminergic neurotoxins (such as 1-methyl-4-phenylpyridinium ion), Cell cycle inhibitors (such as staurosporine), Actinomycins (such as Actinomycin D, dactinomycin), Bleomycins (such as bleomycin A2, bleomycin B2, peplomycin), Anthracyclines (such as daunorubicin, doxorubicin (adriamycin), idarubicin, epirubicin, pirarubicin, zorubicin, mtoxantrone, MDR inhibitors (such as verapamil), Ca²⁺ ATPase inhibitors (such as thapsigargin), Histone deacetylase inhibitors (Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, such as aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfornithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (especially T-2 toxin, verrucarin A, roridin A and anguidine); urethane, siRNA, antisense drugs, and a nucleolytic enzyme.

2). An anti-autoimmune disease agent includes, but is not limited to, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.

3). An anti-infectious disease agent includes, but is not limited to, a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin; b). Amphenicols: azidamfenicol, chloramphenicol, florfenicol, thiamphenicol; c). Ansamycins: geldanamycin, herbimycin; d). Carbapenems: biapenem, doripenem, ertapenem, imipenem/cilastatin, meropenem, panipenem; e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef); f). Glycopeptides: bleomycin, vancomycin (oritavancin, telavancin), teicoplanin (dalbavancin), ramoplanin; g). Glycylcyclines: e. g. tigecycline; g). β-Lactamase inhibitors: penam (sulbactam, tazobactam), clavam (clavulanic acid); i). Lincosamides: clindamycin, lincomycin; j). Lipopeptides: daptomycin, A54145, calcium-dependent antibiotics (CDA); k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin; 1). Monobactams: aztreonam, tigemonam; m). Oxazolidinones: linezolid; n). Penicillins: amoxicillin, ampicillin (pivampicillin, hetacillin, bacampicillin, metampicillin, talampicillin), azidocillin, azlocillin, benzylpenicillin, benzathine benzylpenicillin, benzathine phenoxymethyl-penicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin; o). Polypeptides: bacitracin, colistin, polymyxin B; p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfioxacin, temafloxacin, tosufloxacin, trovafloxacin; q). Streptogramins: pristinamycin, quinupristin/dalfopristin); r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole); s). Steroid antibacterials: e.g. fusidic acid; t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (e.g. tigecycline); u). Other types of antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (e. g. fosfomycin), nitrofurantoin, paclitaxel, platensimycin, pyrazinamide, quinupristin/dalfopristin, rifampicin (rifampin), tazobactam tinidazole, uvaricin;

4). Anti-viral drugs: a). Entry/fusion inhibitors: aplaviroc, maraviroc, vicriviroc, gp41 (enfuvirtide), PRO 140, CD4 (ibalizumab); b). Integrase inhibitors: raltegravir, elvitegravir, globoidnan A; c). Maturation inhibitors: bevirimat, vivecon; d). Neuraminidase inhibitors: oseltamivir, zanamivir, peramivir; e). Nucleosides &nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′, 3′-dideoxynucleoside analogues (e.g. 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (e.g. β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT); f). Non-nucleosides: amantadine, ateviridine, capravirine, diarylpyrimidines (etravirine, rilpivirine), delavirdine, docosanol, emivirine, efavirenz, foscamet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine; g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir; h). Other types of anti-virus drugs: abzyme, arbidol, calanolide a, ceragenin, cyanovirin-n, diarylpyrimidines, epigallocatechin gallate (EGCG), foscamet, griffithsin, taribavirin (viramidine), hydroxyurea, KP-1461, miltefosine, pleconaril, portmanteau inhibitors, ribavirin, seliciclib.

5). The radioisotopes for radiotherapy. Examples of radioisotopes (radionuclides) are ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. Radioisotope labeled antibodies are useful in receptor targeted imaging experiments or can be for targeted treatment such as with the antibody-radioisotope conjugates (Wu et al (2005) Nature Biotechnology 23(9): 1137-46). The cell binding molecules, e.g. an antibody can be labeled with ligand reagents that bind, chelate or otherwise complex a radioisotope metal, using the techniques described in Current Protocols in Immunology, Volumes 1 and 2, Coligen et al, Ed. Wiley-Interscience, New York, Pubs. (1991). Chelating ligands which may complex a metal ion include DOTA, DOTP, DOTMA, DTPA and TETA (Macrocyclics, Dallas, Tex. USA).

6). Another cell-binding molecule-drug conjugate as a synergy therapy. The preferred synergic conjugate can be a conjugate having a cytotoxic agent of a Camptothecin analog, maytansinoid analog, taxanoid (taxane) analog, CC-1065 analog, daunorubicin and doxorubicin compound, amatoxin analog, benzodiazepine dimer (e.g., dimers of pyrrolobenzodiazepine (PBD), tomaymycin, anthramycin, indolinobenzodiazepines, imidazobenzothiadiazepines, or oxazolidinobenzodiazepines), calicheamicins and the enediyne antibiotic compound, actinomycin, azaserine, bleomycins, epirubicin, tamoxifen, idarubicin, dolastatins, auristatins (e.g. monomethyl auristatin E, MMAE, MMAF, auristatin PYE, auristatin TP, Auristatins 2-AQ, 6-AQ, EB (AEB), and EFP (AEFP)), duocarmycins, geldanamycins, methotrexates, thiotepa, vindesines, vincristines, hemiasterlins, nazumamides, microginins, radiosumins, alterobactins, microsclerodermins, theonellamides, esperamicins, PNU-159682, and their analogues and derivatives above thereof.

7). Other immunotherapy drugs: e.g. imiquimod, interferons (e.g. a, P), granulocyte colony-stimulating factors, cytokines, Interleukins (IL-1 IL-35), antibodies (e. g. trastuzumab, pertuzumab, bevacizumab, cetuximab, panitumumab, infliximab, adalimumab, basiliximab, daclizumab, omalizumab, PD-1 or PD-L1), Protein-bound drugs (e.g., Abraxane), an antibody conjugated with drugs selected from calicheamicin derivative, of maytansine derivatives (DM1 and DM4), CC-1065, SN-38, exatecan, topotecan, topoisomerase I inhibitors, duocarmycin, PBD or IGN minor groove binders, potent taxol derivatives, doxorubicin, auristatin antimitotic drugs (e. g. Trastuzumab-DM1, Trastuzumab deruxtecan (DS-8201a), Inotuzumab ozogamicin, Brentuximab vedotin, Sacituzumab govitecan, Glembatumumab vedotin, lorvotuzumab mertansine, AN-152 LMB2, TP-38, VB4-845, Cantuzumab mertansine, AVE9633, SAR3419, CAT-8015 (anti-CD22), IMGN388, Mirvetuximab soravtansine (IMGN853), Enfortumab vedotin, milatuzumab-doxorubicin, SGN-75 (anti-CD70), anti-Her3-exetecan, anti-Trop2-exetecan, nnti-CD79b-MMAE, anti-Her2-MMAE, anti-trop2-MMAE, anti-Her2-MMAF, anti-trop2-MMAF, anti-CD22-calicheamicin derivative, anti-CD22-MMAE, anti-Her2-auristatin derivatives, anti-Muc1-auristatin derivatives, anti-cMet-auristatin derivatives, or anti-Claudin18.2-auristatin derivatives).

8). The pharmaceutically acceptable salts, acids or derivatives of any of the above drugs.

In another synergistic immunotherapy, an antibody of a checkpoint inhibitor, TCR (T cell receptors) T cells, or CARs (chimeric antigen receptors) T cells, or of B cell receptor (BCR), Natural killer (NK) cells, or the cytotoxic cells, or an antibody of anti-CD3, CD4, CD8, CD16 (FcγRIII), CD19, CD20, CD22, CD25, CD27, CD30, CD33, CD37, CD38, CD40, CD40L, CD45RA, CD45RO, CD56, CD57, CD57^{bright}, CD70, CD79, CD79b, CD123, CD125, CD138, TNFβ, Fas ligand, MHC class I molecules (HLA-A, B, C), VEGF, or NKR-P1 antigen is preferred to use along with the conjugates of the present patent for synergistic therapy.

In yet another embodiment, a pharmaceutical composition comprising a therapeutically effective amount of the conjugate of Formula (I)˜(VII) or any conjugates described through the present patent can be administered concurrently with the other therapeutic agents such as the chemotherapeutic agent, the radiation therapy, immunotherapy agents, autoimmune disorder agents, anti-infectious agents or the other conjugates for synergistically effective treatment or prevention of a cancer, or an autoimmune disease, or an infectious disease. The synergistic agents are more preferably selected from one or several of the following drugs: Abatacept, Abiraterone acetate, Abraxane, Acetaminophen/hydrocodone, Acalabrutinib, aducanumab, Adalimumab, ADXS31-142, ADXS-HER2, afatinib dimaleate, aldesleukin, alectinib, alemtuzumab, Alitretinoin, ado-trastuzumab emtansine, Amphetamine/dextroamphetamine, anastrozole, Aripiprazole, anthracyclines, Aripiprazole, Atazanavir, Atezolizumab, Atorvastatin, Avelumab, Axicabtagene ciloleucel, axitinib, belinostat, BCG Live, Bevacizumab, bexarotene, blinatumomab, Bortezomib, bosutinib, brentuximab vedotin, brigatinib, Budesonide, Budesonide/formoterol, Buprenorphine, Cabazitaxel, Cabozantinib, capmatinib, Capecitabine, carfilzomib, chimeric antigen receptor-engineered T (CAR-T) cells, Celecoxib, ceritinib, Cetuximab, Chidamide, Ciclosporin, Cinacalcet, crizotinib, Cobimetinib, Cosentyx, crizotinib, CTL019, Dabigatran, dabrafenib, dacarbazine, daclizumab, dacomotinib, daptomycin, Daratumumab, Darbepoetin alfa, Darunavir, dasatinib, denileukin diftitox, Denosumab, Depakote, Dexlansoprazole, Dexmethylphenidate, Dexamethasone, DigniCap Cooling System, Dinutuximab, Doxycycline, Duloxetine, Duvelisib, durvalumab, elotuzumab, Emtricitabine/Rilpivirine/Tenofovir, disoproxil fumarate, Emtricitbine/tenofovir/efavirenz, Enoxaparin, ensartinib, Enzalutamide, Epoetin alfa, erlotinib, Esomeprazole, Eszopiclone, Etanercept, Everolimus, exemestane, everolimus, exenatide ER, Ezetimibe, Ezetimibe/simvastatin, Fenofibrate, Filgrastim, fingolimod, Fluticasone propionate, Fluticasone/salmeterol, fulvestrant, gazyva, gefitinib, Glatiramer, Goserelin acetate, Icotinib, Imatinib, Ibritumomab tiuxetan, ibrutinib, idelalisib, ifosfamide, Infliximab, imiquimod, ImmuCyst, Immuno BCG, iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, lapatinib, Ipilimumab, Ipratropium bromide/salbutamol, Ixazomib, Kanuma, Lanreotide acetate, lenalidomide, lenaliomide, lenvatinib mesylate, letrozole, Levothyroxine, Levothyroxine, Lidocaine, Linezolid, Liraglutide, Lisdexamfetamine, LN-144, lorlatinib, Memantine, Methylphenidate, Metoprolol, Mekinist, mericitabine/Rilpivirine/Tenofovir, Modafinil, Mometasone, Mycidac-C, Necitumumab, neratinib, Nilotinib, niraparib, Nivolumab, ofatumumab, obinutuzumab, olaparib, Olmesartan, Olmesartan/hydrochlorothiazide, Omalizumab, Omega-3 fatty acid ethyl esters, Oncorine, Oseltamivir, Osimertinib, Oxycodone, palbociclib, Palivizumab, panitumumab, panobinostat, pazopanib, pembrolizumab, PD-1 antibody, PD-L1 antibody, Pemetrexed, pertuzumab, Pneumococcal conjugate vaccine, pomalidomide, Pregabalin, ProscaVax, Propranolol, Quetiapine, Rabeprazole, radium 223 chloride, Raloxifene, Raltegravir, ramucirumab, Ranibizumab, regorafenib, Rituximab, Rivaroxaban, romidepsin, Rosuvastatin, ruxolitinib phosphate, Salbutamol, savolitinib, semaglutide, Sevelamer, Sildenafil, siltuximab, Sipuleucel-T, Sitagliptin, Sitagliptin/metformin, Solifenacin, solanezumab, Sonidegib, Sorafenib, Sunitinib, tacrolimus, tacrimus, Tadalafil, tamoxifen, Tafinlar, Talimogene laherparepvec, talazoparib, Telaprevir, talazoparib, Temozolomide, temsirolimus, Tenofovir/emtricitabine, tenofovir disoproxil fumarate, Testosterone gel, Thalidomide, TICE BCG, Tiotropium bromide, Tisagenlecleucel, toremifene, trametinib, Trastuzumab, Trabectedin (ecteinascidin 743), trametinib, tremelimumab, Trifluridine/tipiracil, Tretinoin, Uro-BCG, Ustekinumab, Valsartan, veliparib, vandetanib, vemurafenib, venetoclax, vorinostat, ziv-aflibercept, Zostavax, and their analogs, derivatives, pharmaceutically acceptable salts, carriers, diluents, or excipients thereof, or a combination above thereof.

According to a still further object, the present invention is also concerned with the process of preparation of the conjugate of the invention. The conjugate and process of the present invention may be prepared in a number of ways well known to those skilled in the art. The Camptothecin analogs used in the conjugate can be synthesized, for example, by application or adaptation of the methods described below, or variations thereon as appreciated by the skilled artisan. The appropriate modifications and substitutions will be readily apparent and well known or readily obtainable from the scientific literature to those skilled in the art. In particular, such methods can be found in R. C. Larock, Comprehensive Organic Transformations, 2^{nd} Edition, Wiley-VCH Publishers, 1999.

In the reactions described hereinafter, it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see P. G. Wuts and T. W. Greene, Greene's Protective Groups in Organic Synthesis, Wiley-Interscience; 4th edition (2006). Some reactions may be carried out in the presence of a base, or an acid or in a suitable solvent. There is no particular restriction on the nature of the base, acid and solvent to be used in this reaction, and any base, acid or solvent conventionally used in reactions of this type may equally be used here, provided that it has no adverse effect on other parts of the molecule. The reactions can take place over a wide range of temperatures. In general, we find it convenient to carry out the reaction at a temperature of from −80° C. to 150° C. (more preferably from about room temperature to 100° C.). The time required for the reaction may also vary widely, depending on many factors, notably the reaction temperature and the nature of the reagents. However, provided that the reaction is effective under the preferred conditions outlined above, a period of from 3 hours to 20 hours will usually suffice.

The work-up of the reaction can be carried out by conventional means. For example, the reaction products may be recovered by distilling off the solvent from the reaction mixture or, if necessary, after distilling off the solvent from the reaction mixture, pouring the residue into water followed by extraction with a water-immiscible organic solvent and distilling off the solvent from the extract. Additionally, the product can, if desired, be further purified by various well-known techniques, such as recrystallization, reprecipitation or the various chromatography techniques, notably column chromatography or preparative thin layer chromatography. The synthesis of the Camptothecin analogs and their conjugates of this invention are illustrated in the FIGS. 1˜32.

The conjugates of binding molecules with potent Camptothecin analogs are further illustrated but not restricted by the description in the following examples.

### EXPERIMENTAL

The invention is further described in the following examples, which are not intended to limit the scope of the invention. Cell lines described in the following examples were maintained in culture according to the conditions specified by the American Type Culture Collection (ATCC) or Deutsche Sammlung von Mikroorganismen und Zellkulturen GmbH, Braunschweig, Germany (DMSZ), or The Shanghai Cell Culture Institute of Chinese Academy of Science, unless otherwise specified. Cell culture reagents were obtained from Invitrogen Corp., unless otherwise specified. Aminal acids and their derivatives as well as preloaded resins were either from Merck Chemicals International Co, or Synthetech Co., or Peptides International Inc or Chembridge International Co. or Sigma-Aldrich (Merck Co). Some of the linkers, Linkers of NHS ester/Maleimide (AMAS, BMPS, GMBS, MBS, SMCC, EMCS or Sulfo-EMCS, SMPB, SMPH, LC-SMCC, Sulfo-KMUS, SM(PEG)4, SM(PEG)6. SM(PEG)8, SM(PEG)12, SM(PEG)24); NHS ester/Pyridyldithiol (SPDP, LC-SPDP or Sulfo-LC-SPDP, SMPT, Sulfo-LC-SMPT); NHS esters/Haloacetyl (SIA, SBAP, SIAB or Sulfo-SIAB); NHS ester/Diazirine (SDA or Sulfo-SDA, LC-SDA or Sulfo-LC-SDA, SDAD or Sulfo-SDAD); Maleimide/Hydrazide (BMPH, EMCH, MPBH, KMUH); Pyridyldithiol/Hydrazide (PDPH); Isocyanate/Maleimide (PMPI) were purchased from Thermo Fisher Scientific Co. SPDB, SPP linkers were made according to the references (Cumber, A. et al, Bioconjugate Chem., 1992, 3, 397-401) and Trastuzumab of Roche was purchased via a pharmacy in China. Trop-2 antibody is a biosimilar of Sacituzumab, generated in house, and EGFR antibody here is Nimotuzumab, bought from a pharmacy in China. PEG and PEG derivative compounds were purchased from Biomatrik Inc, Jiaxing City, Zhejiang Province, China. Topotecans and their derivatives or major components were bought from several commercial sources, such as from Chengdu Tianyuan Natural Product Co., Ltd, Chengdu, China; Brightgene Biomedical Co., Suzhou, China; etc. Experimental animals were purchased from National Resource Center of Model Mice via GemPharmatech. Co. Ltd, Najing, China and Shanghai SLAC Laboratory Animal Co., Ltd., Shanghai, China; T-DM1 from Roche was purchased from a pharmacy in Hong Kong, China. All other reagents and solvents were purchased as the highest grade available and used without further purification. EDC (EDCI), PFP, HATU, TATU, PyBrOP, DIPEA, TEA, PPTS, DMAP, BrOP, p-TSA, DTT, EDTA, TCEP, NHS, TFA, Ellman reagent, Traut reagent (2-iminothiolane), γ-thiobutyrolactone and all other chemicals as well as anhydrous solvents were from Sigma-Aldrich International (Merck) or Aladdin Chemical (Shanghai) Ltd. All anhydrous solvents were commercially obtained and stored in Sure-seal bottles under nitrogen. The preparative HPLC (acetonitrile/water containing formic acid or TFA) separations were performed with Varain PreStar HPLC. NMR spectra were recorded on Bruker 500 MHz Instrument. Chemical shifts (delta) are reported in parts per million (ppm) referenced to tetramethylsilane at 0.00 and coupling constants (J) are reported in Hz. The mass spectral data were acquired on a Waters Xevo QTOF mass spectrum equipped with Waters Acquity UPLC separations module and Acquity TUV detector.

### Example 1. Synthesis of (S)-tert-butyl (1-((4-ethyl-4,9-dihydroxy-3,14-dioxo-3,4,12,14-tetrahydro-1H-pyrano[3′,4′:6,7]indolizino[1,2-b]quinolin-10-yl)methyl)piperidin-4-yl)carbamate (2)

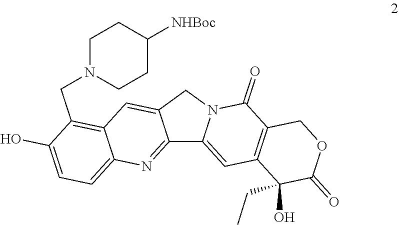 [SMILES: CC[C@@]1(O)C(=O)OCc2c1cc1n(c2=O)Cc2cc3c(CN4CCC(NC(=O)OC(C)(C)C)CC4)c(O)ccc3nc2-1]

To a solution of 10-hydroxycamptothecin (2 g, 5.49 mmol) in acetic acid (10 mL) was added a solution of 4-tert-butoxycarbonylaminopiperidine (4.4 g, 21.9 mmol) and 37% formaldehyde (1.8 g, 21.9 mmol) in acetic acid (15 mL). The reaction mixture was heated to about 60° C. and stirred for 2 hours, then acetic acid was removed. Recrystallization in 10 mL of MeOH gave compound 2 (2.1 g, 68% yield) as a yellow powdery solid. ESI-MS m/z: [M+H]⁺ calcd for C₃₁H₃₆N₄O₇: 577.26. found 577.26.

### Example 2. Synthesis of (S)-10-((4-aminopiperidin-1-yl)methyl)-4-ethyl-4,9-dihydroxy-1H-pyrano[3′,4′:6,7]indolizino[1,2-b]quinoline-3,14(4H,12H)-dione (3)

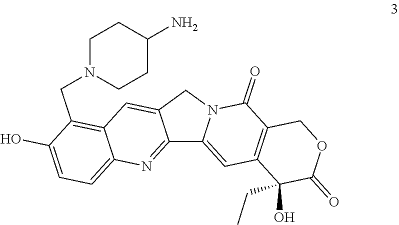 [SMILES: CC[C@@]1(O)C(=O)OCc2c1cc1n(c2=O)Cc2cc3c(CN4CCC(N)CC4)c(O)ccc3nc2-1]

Compound 2 (150 mg, 0.26 mmol) was dissolved in a mixture of dichloromethane and trifluoroacetic acid (2 mL/6 mL), and stirred at r.t. for 1 hour. The mixture was then concentrated and dried on a vacuum pump to give compound 3 (120 mg, 100% yield) as a yellow solid. ESI-MS m/z: [M+H]⁺ calcd for C₂₆H₂₈N₄O₅: 477.21. found 477.21.

### Example 3. Synthesis of Compound (S)-4-(2,5-dioxo-2,5-dihydro-1H-pyrrol-1-yl)-N-(1-((4-ethyl-4,9-dihydroxy-3,14-dioxo-3,4,12,14-tetrahydro-1H-pyrano[3′,4′:6,7]indolizino[1,2-b]quinolin-10-yl)methyl)piperidin-4-yl)butanamide (5)

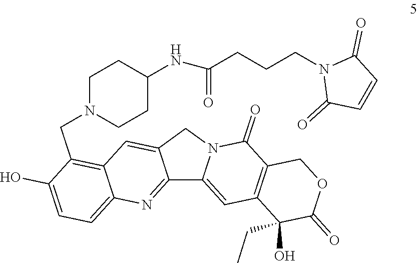 [SMILES: CC[C@@]1(O)C(=O)OCc2c1cc1n(c2=O)Cc2cc3c(CN4CCC(NC(=O)CCCN5C(=O)C=CC5=O)CC4)c(O)ccc3nc2-1]

Compound 3 (62 mg, 0.13 mmol) and perfluorophenyl 4-(2,5-dioxo-2,5-dihydro-1H-pyrrol-1-yl)butanoate (compound 4, 56 mg, 0.16 mmol) were dissolved in DMF (5 mL), cooled to about 0° C., and then N, N-diisopropylethylamine (45 μL, 0.16 mmol) was added. The reaction was warmed to r.t. and stirred for 2 hours, concentrated, and purified by preparative HPLC (acetonitrile/water containing formic acid) to give compound 5 (44 mg, 54% yield). ESI-MS m/z: [M+H]⁺ calcd for C₃₄H₃₅N₅O₈: 642.25. found 642.25.

### Example 4. Synthesis of (S)-perfluorophenyl 30-(4-(2,5-dioxo-2,5-dihydro-1H-pyrrol-1-yl)butanamido)-27,31-dioxo-2,5,8,11,14,17,20,23-octaoxa-26,32-diazahexatriacontan-36-oate (7)

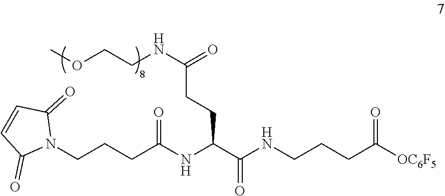

To a solution of compound 6 (100 mg, 0.13 mmol) dissolved in dichloromethane (5 mL), were added pentafluorophenol (48 mg, 0.26 mmol) and 1-(3-dimethylaminopropyl)-3-ethylcarbodiimide hydrochloride (50 mg, 0.26 mmol). The reaction was stirred at r.t. for 2 hours and diluted with dichloromethane (50 mL), washed with water (2×10 mL), dried over sodium sulfate, filtered, and concentrated to give compound 7 (120 mg, 100% yield). ESI-MS m/z: [M+H]⁺ calcd for C₄₀H₅₇F₅N₄O₁₅: 929.37. found 929.37.

### Example 5. Synthesis of(S)-2-(4-(2,5-dioxo-2,5-dihydro-1H-pyrrol-1-yl)butanamido)-N1-(4-((1-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)piperidin-4-yl)amino)-4-oxobutyl)-N5-(2,5,8,11,14,17,20,23-octaoxapentacosan-25-yl)pentanediamide (8)

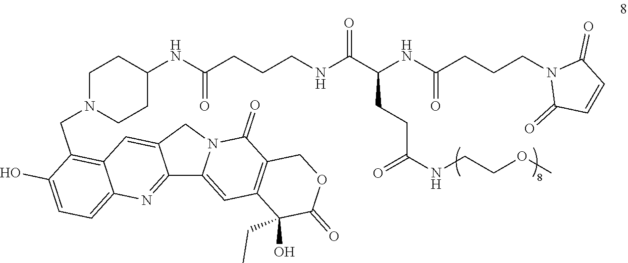

Compound 3 (60 mg, 0.126 mmol) and compound 7 (97 mg, 0.105 mmol) were dissolved in DMF (5 mL), cooled to about 0° C., and then N, N-diisopropylethylamine (37 μL, 0.21 mmol) was added. The reaction was warmed to r.t. and stirred for 2 hours, concentrated, and purified by preparative HPLC (acetonitrile/water containing formic acid) to give compound 8 (50 mg, 39% yield). ESI-MS m/z: [M+H]⁺ calcd for C₆₀H₈₄N₈O₁₉: 1221.59. found 1221.59.

### Example 6. Synthesis of tert-butyl 3-(2-(2-(4-(2,5-dioxo-2,5-dihydro-1H-pyrrol-1-yl)butanamido)ethoxy)ethoxy)propanoate (10)

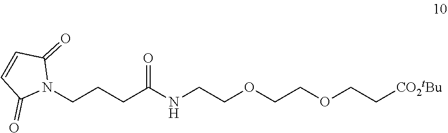 [SMILES: CC(C)(C)OC(=O)CCOCCOCCNC(=O)CCCN1C(=O)C=CC1=O]

Compound 9 (1.32 g, 5.7 mmol) was dissolved in DMF (10 mL), to which 4-(2,5-dioxo-2,5-dihydro-1H-pyrrol-1-yl)butanoic acid (1.04 g, 5.7 mmol) was added, followed by HATU (2.6 g, 6.8 mmol) and triethylamine (0.96 mL, 6.8 mmol) in sequence, and the reaction was stirred at r.t. for 1 h, diluted with dichloromethane (100 mL), washed with water (2×10 mL), dried over anhydrous sodium sulfate, filtered and concentrated. The residue was purified by column chromatography (ethyl acetate/petroleum ether) to give compound 10 (1.8 g, 80% yield). ESI-MS m/z: [M+H]⁺ calcd for C₁₉H₃₀N₂O₇: 399.21. found 399.21.

### Example 7. Synthesis of 3-(2-(2-(4-(2,5-dioxo-2,5-dihydro-1H-pyrrol-1-yl)butanamido)ethoxy)ethoxy)propanoic acid (11)

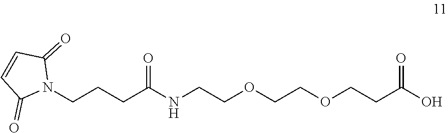 [SMILES: O=C(O)CCOCCOCCNC(=O)CCCN1C(=O)C=CC1=O]

Compound 10 (0.40 g, 1.0 mmol) was dissolved in a mixture of dichloromethane and trifluoroacetic acid (12 mL/4 mL), and stirred at r.t. for 1 hour. The mixture was then concentrated, co-evaporated with dichloromethane twice and dried on a vacuum pump to give compound 11 (0.34 g, 100% yield) as a yellow solid. ESI-MS m/z: [M+H]⁺ calcd for C₁₅H₂₂N₂O₇: 343.14. found 343.14.

### Example 8. Synthesis of perfluorophenyl 3-(2-(2-(4-(2,5-dioxo-2,5-dihydro-1H-pyrrol-1-yl)butanamido)ethoxy)ethoxy)propanoate (12)

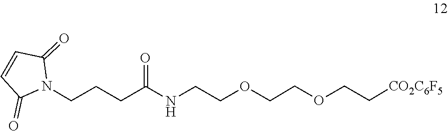

To a solution of compound 11 (0.34 g, 1.0 mmol) dissolved in dichloromethane (30 mL), were added pentafluorophenol (0.46 g, 2.5 mmol) and 1-(3-dimethylaminopropyl)-3-ethylcarbodiimide hydrochloride (0.57 g, 3.0 mmol). The reaction was stirred at r.t. for 2 hours and diluted with dichloromethane (50 mL), washed with water (200 mL), dried over sodium sulfate, filtered, and concentrated to give compound 12 (0.51 g, 100% yield). ESI-MS m/z: [M+H]⁺ calcd for C₂₁H₂₂F₅N₂O₇: 509.13. found 509.13.

### Example 9. Synthesis of N,N-dimethylpiperidin-4-amine (13)

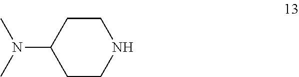

N-Boc piperidone (10 g, 0.05 mol) was dissolve in MeOH (100 mL), to which dimethylamine aqueous solution (25 mL, 0.22 mol) and 10% palladium on carbon (1 g) were added, and the reaction flask was evacuated and re-filled with hydrogen, then stirred at r.t. overnight. After filtration, the filtrate was concentrated and co-evaporated with dichloromethane for three times (3×80 mL), and dried on a vacuum pump to remove all dimethylamine. HCl/MeOH (4 M, 50 mL) was added to the residue and stirred at r.t. for 30 minutes. A large amount of white solid precipitated out and the mixture was filtered to yield a white solid 13 (9 g, 90% yield). ESI-MS m/z: [M+H]⁺ calcd for C₇H₁₆N₂, 129.13. found 129.13.

### Example 10. Synthesis of (9H-fluoren-9-yl)methyl 4-(dimethylamino)piperidine-1-carboxylate (14)

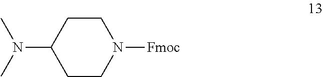

Compound 13 (2.0 g, 9.9 mmol) was dissolved in a mixed solution of 1,4-dioxane and water (30 mL/50 mL), and sodium bicarbonate (2.5 g, 29.8 mmol) was added, and the mixture was cooled to 0° C. A solution of 9-fluorenylmethoxycarbonyl chloride (3.1 g, 11.9 mmol) in 1,4-dioxane (10 mL) was added dropwise. After the addition, the temperature was gradually raised to r.t. and the reaction was stirred for 1 hour. 100 mL of 1M HCl was added, and the mixture was washed with ethyl acetate (3×50 mL), the aqueous phase was adjusted to pH˜10 with sodium carbonate, then extracted with dichloromethane (3×50 mL). The combined organic phases were washed with water (50 mL), dried over sodium sulfate, filtered, concentrated, and purified by column chromatography (MeOH/dichloromethane) to yield compound 14 (2.75 g, 79% yield). ESI-MS m/z: [M+H]⁺ calcd for C₂₂H₂₆N₂O₂, 351.20. found 351.20.

### Example 11. Synthesis of (S)-tert-butyl (1-((4-(hydroxymethyl)phenyl)amino)-1-oxopropan-2-yl)carbamate (15)

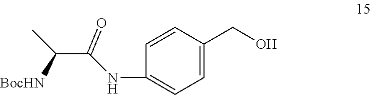 [SMILES: C[C@H](NC(=O)OC(C)(C)C)C(=O)Nc1ccc(CO)cc1]

p-aminobenzyl alcohol (5.0 g, 0.04 mol) and Boc-L-alanine (8.0 g, 0.042 mol) were dissolved in anhydrous THF (100 mL), and 2-ethoxy-1-ethoxycarbonyl-1,2-dihydroquinoline (11 g, 0.044 mol) was added and stirred at r.t. overnight. The reaction mixture was poured into water (300 mL), extracted with ethyl acetate (3×100 mL), the combined organic phases were washed with water (100 mL), dried over sodium sulfate, filtered, and concentrated. The crude product was triturated with ethyl acetate/petroleum ether (1:3) and filtered to yield compound 15 (9.8 g, 84% yield) as a white solid. ESI-MS m/z: [M+H]⁺ calcd for C₁₅H₂₂N₂O₄: 295.16. found 295.16.

### Example 12. Synthesis of (S)-tert-butyl (1-((4-(bromomethyl)phenyl)amino)-1-oxopropan-2-yl)carbamate (16)

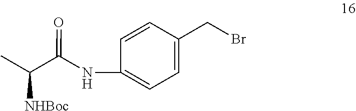 [SMILES: C[C@H](NC(=O)OC(C)(C)C)C(=O)Nc1ccc(CBr)cc1]

Compound 3 (3.5 g, 11.9 mmol) and carbon tetrabromide (5.9 g, 17.8 mmol) were dissolved in dichloromethane (80 mL), cooled to about 0° C., and triphenylphosphine (4.7 g, 17.8 mmol) was added. The reaction was warmed to r.t. and stirred for 30 minutes, and then 20 g of silica gel was added, mixed, and dried on a rotavap, loaded on a silica gel column (100 g of silica gel) and eluted with petroleum ether/ethyl acetate to yield compound 16 (2.6 g, 62% yield). ESI-MS m/z: [M+H]⁺ calcd for C₁₅H₂₁BrN₂O₃: 357.07. found 357.07.

### Example 13. Synthesis of(S)-1-(((9H-fluoren-9-yl)methoxy)carbonyl)-N-(4-(2-((tert-butoxycarbonyl)amino)propanamido)benzyl)-N,N-dimethylpiperidin-4-aminium bromide (17)

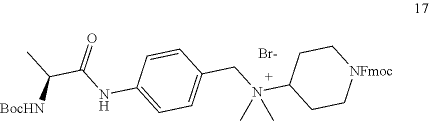

Compound 16 (2.3 g, 6.4 mmol) and compound 14 (2.7 g, 7.7 mmol) were dissolved in anhydrous THF (50 mL) and stirred at r.t. overnight. After removal of most THF on a rotavap, ethyl acetate (50 mL) was added to the residue. The resulting slurry was filtered to give a white solid (4.5 g, 100% yield). ESI-MS m/z: M+ calcd for C₃₇H₄₇N₄O₅: 627.35. found 627.35.

### Example 14. Synthesis of (S)-N-(4-(2-((tert-butoxycarbonyl)amino)propanamido)benzyl)-N,N-dimethylpiperidin-4-aminium bromide (18)

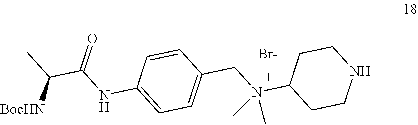

Compound 17 (1.0 g, 1.41 mmol) was dissolved in DMF (5 mL), and piperidine (1 mL) was added. After stirring at r.t. for 30 minutes, 30 mL of ethyl acetate was added and stirred for 10 minutes. The mixture was filtered to give a white powdery solid (550 mg, 80% yield). ESI-MS m/z: M⁺ calcd for C₂₂H₃₇N₄O₃: 405.29. found 405.29.

### Example 15. Synthesis of N-(4-((S)-2-((tert-butoxycarbonyl)amino)propanamido)benzyl)-1-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)-N,N-dimethylpiperidin-4-aminium bromide (19)

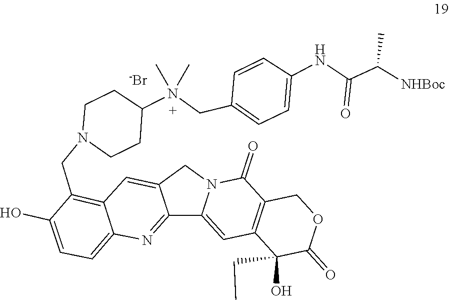

To a solution of 10-hydroxycamptothecin (375 mg, 1.03 mmol) in acetic acid (5 mL) was added a solution of compound 18 (550 mg, 1.13 mmol) and 37% formaldehyde (92 mg, 1.13 mmol) in acetic acid (5 mL). The mixture was heated to about 65° C. and stirred for 1 hour, then concentrated, co-evaporated with dry MeOH. Recrystallization in dichloromethane and a small amount of MeOH gave compound 19 (0.5 g, 63% yield) as a yellow powder. ESI-MS m/z: M⁺ calcd for C₄₃H₅₃N₆O₈: 781.39. found 781.39.

### Example 16. Synthesis of N-(4-((S)-2-aminopropanamido)benzyl)-1-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)-N,N-dimethylpiperidin-4-aminium bromide (20)

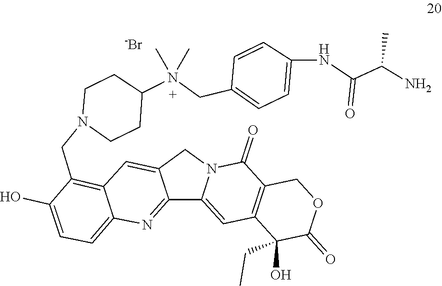

Compound 19 (50 mg, 0.058 mmol) was dissolved in a mixture of dichloromethane and trifluoroacetic acid (2 mL/6 mL), and stirred at r.t. for 30 minutes. The mixture was then concentrated and dried on a vacuum pump to give compound 20 (44 mg, 100% yield) as a yellow solid. ESI-MS m/z: M⁺ calcd for C₃₈H₄₅N₆O₆: 681.34. found 681.34.

### Example 17. Synthesis of N-(4-((S)-2-(4-(2,5-dioxo-2,5-dihydro-1H-pyrrol-1-yl)butanamido)propanamido)benzyl)-1-(((S)-4-ethyl-4,9-dihydroxy-3,14-dioxo-3,4,12,14-tetrahydro-TH-pyrano[3′,4′:6,7]indolizino[1,2-b]quinolin-10-yl)methyl)-N,N-dimethylpiperidin-4-aminium formate (21)

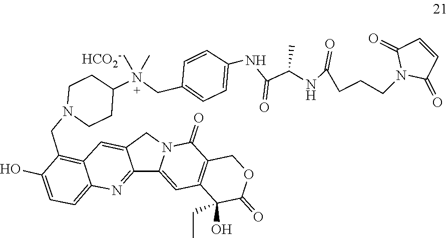

Compound 20 (88 mg, 0.116 mmol) and compound 4 (49 mg, 0.140 mmol) were dissolved in DMF (5 mL), cooled to about 0° C., and then N, N-diisopropylethylamine (40 μL, 0.232 mmol) was added. The reaction was warmed to r.t. and stirred for 2 hours, concentrated, and purified by preparative HPLC (acetonitrile/water containing formic acid) to give compound 21 (66 mg, 68% yield). ESI-MS m/z: M⁺ calcd for C₄₆H₅₂N₇O₉: 846.38. found 846.38.

### Example 18. Synthesis of N-(4-((9S,17S)-9-(4-(2,5-dioxo-2,5-dihydro-TH-pyrrol-1-yl)butanamido)-17-methyl-6,10,15-trioxo-2-oxa-5,11,16-triazaoctadecanamido)benzyl)-1-(((S)-4-ethyl-4,9-dihydroxy-3,14-dioxo-3,4,12,14-tetrahydro-1H-pyrano[3′,4′:6,7]indolizino[1,2-b]quinolin-10-yl)methyl)-N,N-dimethylpiperidin-4-aminium formate (22)

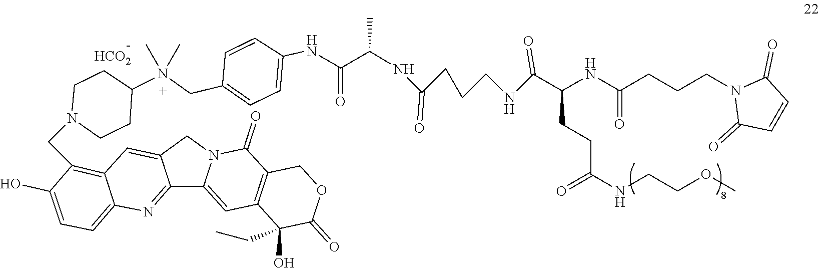

Compound 20 (44 mg, 0.058 mmol) and compound 7 (60 mg, 0.065 mmol) were dissolved in DMF (5 mL), cooled to about 0° C., and then N, N-diisopropylethylamine (20 μL, 0.116 mmol) was added. The reaction was warmed to r.t. and stirred for 2 hours, concentrated, and purified by preparative HPLC (acetonitrile/water containing formic acid) to give compound 22 (51 mg, 58% yield). ESI-MS m/z: M+ calcd for C₇₂H₁₀₁N₁₀O₂₀: 1425.72. found 1425.72.

### Example 19. Synthesis of 1-(2-amino-4-fluoro-5-methoxyphenyl)-2-chloroethanone (24d)

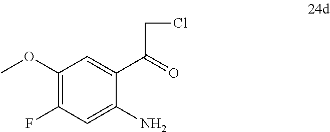

A solution of 3-fluoro-4-methoxyaniline (5 g, 35.4 mmol) dissolved in dichloromethane (20 mL) was added dropwise to an ice water cooled boron trichloride (1 M in dichloromethane, 38.9 mL) solution. The reaction was stirred for 10 minutes and then chloroacetonitrile (3.2 g, 42.5 mmol) and aluminum trichloride (5.2 g, 38.9 mmol) were added. After the addition was completed, the reaction was warmed to r.t. and then refluxed overnight. The reaction mixture was then cooled to about 0° C., quenched with 2 M HCl (80 mL) and stirred at r.t. for 2 hours. Layers were separated and the aqueous phase was extracted with dichloromethane (3×80 mL). Combined organic phases were washed with water (100 mL), dried over sodium sulfate, filtered, concentrated, purified on a silica gel column, eluted with petroleum ether/ethyl acetate to give compound 24d (2 g, 26% yield) as a yellow solid. ESI-MS m/z: [M+H]⁺ calcd for C₉H₉ClFNO₂: 218.03. found 218.03.

### Example 20. Synthesis of (S)-11-(chloromethyl)-4-ethyl-8-fluoro-4-hydroxy-9-methoxy-1H-pyrano[3′,4′:6,7]indolizino[1,2-b]quinoline-3,14(4H,12H)-dione (26d)

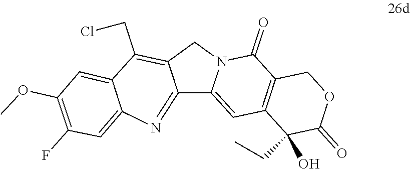 [SMILES: CC[C@@]1(O)C(=O)OCc2c1cc1n(c2=O)Cc2c-1nc1cc(F)c(OC)cc1c2CCl]

Compound 24d (0.50 g, 2.29 mmol) and compound 25 (0.57 g, 2.19 mmol) were dissolved in anhydrous toluene (40 mL), and p-toluenesulfonic acid (42 mg, 0.219 mmol) was added. The suspension was heated at reflux for 2 days and allowed to cool to r.t. After removal of about two-thirds of toluene, the residue was filtered and the filter cake was washed with dichloromethane, air-dried to give compound 26d (0.7 g, 72% yield) as a gray powdery solid. ESI-MS m/z: [M+H]⁺ calcd for C₂₂H₁₈ClFN₂O₅: 445.09. found 445.09.

### Example 21. Synthesis of N-(4-((S)-2-((tert-butoxycarbonyl)amino)propanamido)benzyl)-1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium chloride (27)

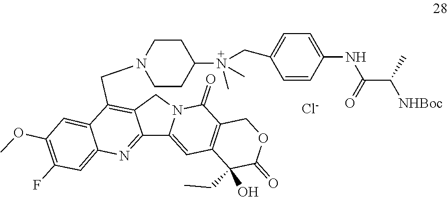

A mixture of compound 26d (218 mg, 0.49 mmol), compound 18 (200 mg, 0.49 mmol) in DMF (5 mL) was stirred at 0° C. for 30 minutes, then triethylamine (63 μL, 0.45 mmol) was added and the stirring was continued for 1 hour. The reaction was concentrated and purification by preparative HPLC (acetonitrile/water containing formic acid) gave compound 10 (240 mg, 59% yield) as a yellow solid. ESI-MS m/z: M+ calcd for C₄₄H₅₄FN₆O₈: 813.40. found 813.40.

### Example 22. Synthesis of N-(4-((S)-2-aminopropanamido)benzyl)-1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium (28)

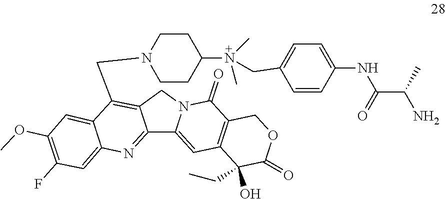

Compound 27 (50 mg, 0.06 mmol) was dissolved in a mixture of dichloromethane and trifluoroacetic acid (2 mL/6 mL), and stirred at r.t. for 30 minutes. The mixture was then concentrated and dried on a vacuum pump to give compound 28 (42 mg, 100% yield) as a yellow solid. ESI-MS m/z: M+ calcd for C₃₉H₄₆FN₆O₆: 713.35. found 713.35.

### Example 23. Synthesis of N-(4-((30S,38S)-30-(4-(2,5-dioxo-2,5-dihydro-1H-pyrrol-1-yl)butanamido)-38-methyl-27,31,36-trioxo-2,5,8,11,14,17,20,23-octaoxa-26,32,37-triazanonatriacontanamido)benzyl)-1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium formate (29)

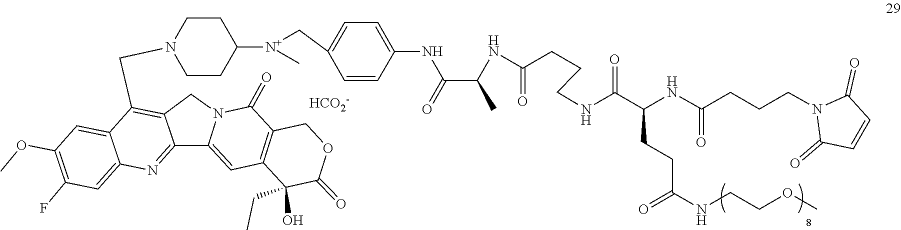

Compound 28 (47 mg, 0.060 mmol) and compound 7 (60 mg, 0.066 mmol) were dissolved in DMF (5 mL), cooled to about 0° C., and then N, N-diisopropylethylamine (21 μL, 0.12 mmol) was added. The reaction was warmed to r.t. and stirred for 2 hours, concentrated, and purified by preparative HPLC (acetonitrile/water containing formic acid) (acetonitrile/water containing formic acid) to give compound 29 (23 mg, 25% yield). ESI-MS m/z: M+ calcd for C₇₃H₁₀₂FN₁₀O₂₀: 1457.73. found 1457.73.

### Example 24. Synthesis of (S)-11-(aminomethyl)-4-ethyl-8-fluoro-4-hydroxy-9-methoxy-1H-pyrano[3′,4′:6,7]indolizino[1,2-b]quinoline-3,14(4H,12H)-dione (30)

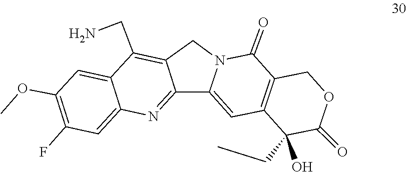 [SMILES: CC[C@@]1(O)C(=O)OCc2c1cc1n(c2=O)Cc2c-1nc1cc(F)c(OC)cc1c2CN]

Compound 26d (80 mg, 0.18 mmol) was dissolved in ethanol (5 mL), hexamethylenetetramine (76 mg, 0.54 mmol) was added and the mixture was refluxed for 90 minutes and then cooled to r.t. Concentrated hydrochloric acid (100 μL) was added, and stirred for 30 minutes. After concentration, an off-white solid was obtained, which was purified by preparative HPLC (acetonitrile/water containing formic acid) to give compound 30 (40 mg, 52% yield). ESI-MS m/z: [M+H]⁺ calcd for C₂₂H₂₀FN₃O: 426.14. found 426.14.

### Example 25. Synthesis of (S)-2-(4-(2,5-dioxo-2,5-dihydro-1H-pyrrol-1-yl)butanamido)-N1-(4-((((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-10-yl)methyl)amino)-4-oxobutyl)-N5-(2,5,8,11,14,17,20,23-octaoxapentacosan-25-yl)pentanediamide (31)

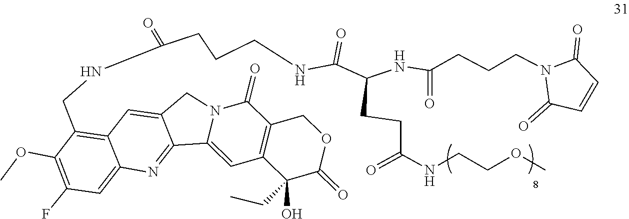

Compound 30 (40 mg, 0.094 mmol) and compound 7 (120 mg, 0.13 mmol) were dissolved in DMF (5 mL), cooled to about 0° C., and then N, N-diisopropylethylamine (33 μL, 0.188 mmol) was added. The reaction was warmed to r.t. and stirred for 2 hours, concentrated, and purified by preparative HPLC (acetonitrile/water containing formic acid) to give compound 31 (55 mg, 50% yield). ESI-MS m/z: [M+H]⁺ calcd for C₅₆H₇₆FN₇O₁₉: 1170.52. found 1170.52.

### Example 26. Synthesis of tert-butyl (1-methylpiperidin-4-yl)carbamate (32)

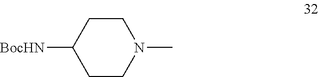

4-(tert-butoxycarbonylamino)piperidine (2 g, 10 mmol) was dissolved in MeOH (30 mL), and then 37% formaldehyde (1.6 g, 20 mmol) and 10% palladium on carbon (0.2 g) were added. The reaction was stirred under 1 atm hydrogen overnight and filtered. The filtrate was concentrated to give compound 32 (2.1 g, 100% yield). ESI-MS m/z: [M+H]⁺ calcd for C₁₁H₂₂N₂O₂: 215.17. found 215.17.

### Example 27. Synthesis of (S)-4-((tert-butoxycarbonyl)amino)-1-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperidin-1-ium chloride (33)

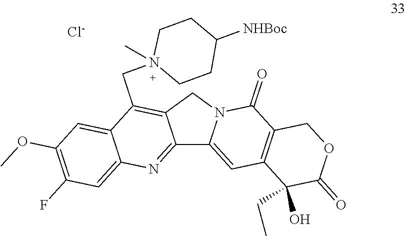

Compound 26d (50 mg, 0.112 mmol) and compound 32 (26 mg, 0.123 mmol) in DMF (3 mL) was stirred at r.t. for 2 hours. The reaction solution was purified by preparative HPLC (acetonitrile/water containing formic acid) to give compound 6 (33 mg, 47% yield). ESI-MS m/z: [M]⁺ calcd for C₃₃H₄₀FN₄O₇: 623.29. found 623.29.

### Example 28. Synthesis of (S)-4-amino-1-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperidin-1-ium (34)

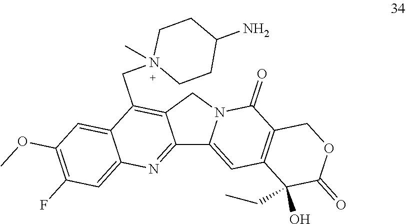

Compound 33 (30 mg, 0.053 mmol) was dissolved in a mixture of dichloromethane and trifluoroacetic acid (3 mL/1 mL), and stirred at r.t. for 30 minutes. The mixture was then concentrated and dried on a vacuum pump to give compound 34 (33 mg, 100% yield). ESI-MS m/z: [M]⁺ calcd for C₂₈H₃₂N₄O₅: 477.21. found 477.21.

### Example 29. Synthesis of 4-((S)-30-(4-(2,5-dioxo-2,5-dihydro-1H-pyrrol-1-yl)butanamido)-27,31-dioxo-2,5,8,11,14,17,20,23-octaoxa-26,32-diazahexatriacontanamido)-1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperidin-1-ium formate (35)

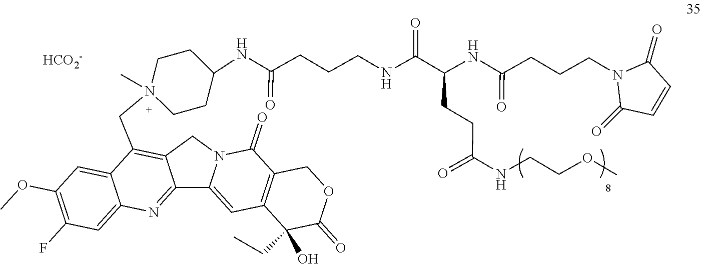

Compound 34 (30 mg, 0.053 mmol) and compound 7 (60 mg, 0.079 mmol) were dissolved in DMF (5 mL), cooled to about 0° C., and then N, N-diisopropylethylamine (18 μL, 0.106 mmol) was added. The reaction was warmed to r.t. and stirred for 2 hours, concentrated, and purified by preparative HPLC (acetonitrile/water containing formic acid) to give compound 35 (15 mg, 21% yield). ESI-MS m/z: [M]⁺ calcd for C₆₂H₈₈FN₈O₁₉: 1267.61. found 1267.61.

### Example 30. Synthesis of (9H-fluoren-9-yl)methyl 4-methylpiperazine-1-carboxylate (36)

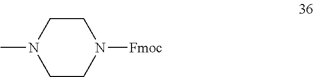

1-methylpiperazine (5.0 g, 50.0 mmol) was dissolved in a mixed solution of 1,4-dioxane and water (60 mL/100 mL), and sodium bicarbonate (12.6 g, 150 mmol) was added, and the mixture was cooled to 0° C. A solution of 9-fluorenylmethoxycarbonyl chloride (15.5 g, 60.0 mmol) in 1,4-dioxane (20 mL) was added dropwise. After the addition, the temperature was gradually raised to r.t. and the reaction was stirred for 3 hours. 300 mL of 1M HCl was added, and the mixture was washed with ethyl acetate (2×100 mL), the aqueous phase was adjusted to pH˜10 with sodium carbonate, then extracted with ethyl acetate (2×100 mL). The combined organic phases were washed with water (250 mL), dried over sodium sulfate, filtered, concentrated, and purified by column chromatography (MeOH/dichloromethane) to yield compound 2 (6.5 g, 40% yield). ESI-MS m/z: [M+H]⁺ calcd for C₂₀H₂₂N₂O₂, 323.17. found 323.19.

### Example 31. Synthesis of(S)-4-(((9H-fluoren-9-yl)methoxy)carbonyl)-1-(4-(2-((tert-butoxycarbonyl)amino)propanamido)benzyl)-1-methylpiperazin-1-ium bromide (37)

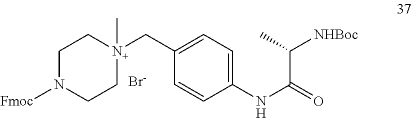

Compound 16 (2.3 g, 6.4 mmol) and compound 36 (2.1 g, 6.4 mmol) were dissolved in anhydrous THF (100 mL) and stirred at r.t. overnight. After removal of most THF on a rotavap, ethyl acetate (200 mL) was added to the residue. The resulting slurry was filtered to give a white solid (3.8 g, 87% yield). ESI-MS m/z: M+ calcd for C₃₅H₄₃N₄O₅: 599.32. found 599.32.

### Example 32. Synthesis of (S)-1-(4-(2-((tert-butoxycarbonyl)amino)propanamido)benzyl)-1-methylpiperazin-1-ium bromide (38)

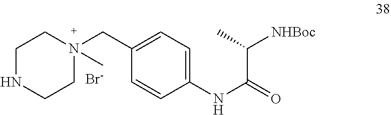

Compound 37 (3.12 g, 4.6 mmol) was dissolved in DMF (25 mL), and piperidine (3 mL) was added. After stirring at r.t. for 2 hours, 200 mL of ethyl acetate was added and stirred for 10 minutes. The mixture was filtered to give a white solid (1.54 g, 77% yield). ESI-MS m/z: M⁺ calcd for C₂₀H₃₃N₄O₃: 377.26. found 377.26.

### Example 33. Synthesis of 1-(4-((S)-2-((tert-butoxycarbonyl)amino)propanamido)benzyl)-4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium (39)

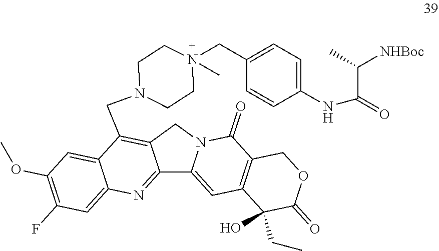

A mixture of compound 38 (0.30 g, 0.66 mmol), compound 26d (0.25 g, 0.56 mmol) in DMF (10 mL) was stirred at 0° C. for 30 minutes, then N, N-diisopropylethylamine (49 μL, 0.28 mmol) was added and the reaction was warmed to r.t. and stirred overnight, concentrated and purification by preparative HPLC (acetonitrile/water containing formic acid) to give compound 39 (0.40 g, 80% yield). ESI-MS m/z: M⁺ calcd for C₄₂H₅₀FN₆O₈: 785.37. found 785.37.

### Example 34. Synthesis of 1-(4-((S)-2-aminopropanamido)benzyl)-4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium (40)

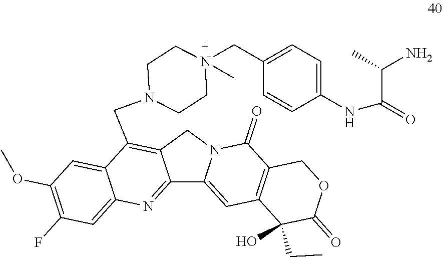

Compound 39 (0.30 g, 0.35 mmol) was dissolved in a mixture of dichloromethane and trifluoroacetic acid (3 mL/3 mL), and stirred at r.t. for 30 minutes. The mixture was then concentrated and dried on a vacuum pump to give compound 40 (0.27 g, 100% yield) as a yellow solid. ESI-MS m/z: M+ calcd for C₃₇H₄₂FN₆O₆: 477.21. found 477.21.

### Example 35. Synthesis of 1-(4-((S)-2-(4-(2,5-dioxo-2,5-dihydro-1H-pyrrol-1-yl)butanamido)propanamido)benzyl)-4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium formate (41)

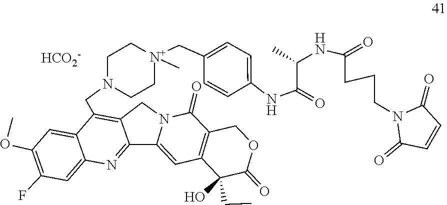 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)CCCN3C(=O)C=CC3=O)cc2)CC1 ; "HCO2-"]

Compound 40 (50 mg, 0.065 mmol) and compound 4 (30 mg, 0.098 mmol) were dissolved in DMF (3 mL), and then N, N-diisopropylethylamine (45 μL, 0.26 mmol) was added. The reaction was stirred at r.t for 30 minutes, concentrated, and purified by preparative C-18 HPLC (acetonitrile/water containing formic acid) to give compound 41 (37 mg, 61% yield). ESI-MS m/z: M⁺ calcd for C₄₅H₄₉FN₇O₉: 850.36. found 850.36.

### Example 36. Synthesis of 1-(4-((30S,38S)-30-(4-(2,5-dioxo-2,5-dihydro-1H-pyrrol-1-yl)butanamido)-38-methyl-27,31,36-trioxo-2,5,8,11,14,17,20,23-octaoxa-26,32,37-triazanonatriacontanamido)benzyl)-4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium formate (42)

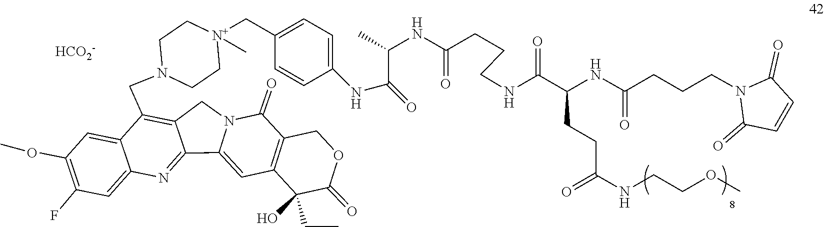

Compound 40 (70 mg, 0.092 mmol) was dissolved in DMF (2 mL), to which compound 7 (70 mg, 0.092 mmol) in DMF (2 mL) was added, followed by HATU (52 mg, 0.138 mmol) and triethylamine (52 μL, 0.368 mmol) in sequence, and the reaction was stirred at r.t. for 30 minutes. After concentration, the residue was purified by preparative HPLC (acetonitrile/water containing formic acid) to give compound 43 (50.9 mg, 37% yield). ESI-MS m/z: [M+]⁺ calcd for C₇₁H₉₈FN₁₀O₂₀: 1429.69. found 1429.69.

### Example 37. Synthesis of 1-(4-((S)-17-(2,5-dioxo-2,5-dihydro-1H-pyrrol-1-yl)-2-methyl-4,14-dioxo-7,10-dioxa-3,13-diazaheptadecanamido)benzyl)-4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium formate (43)

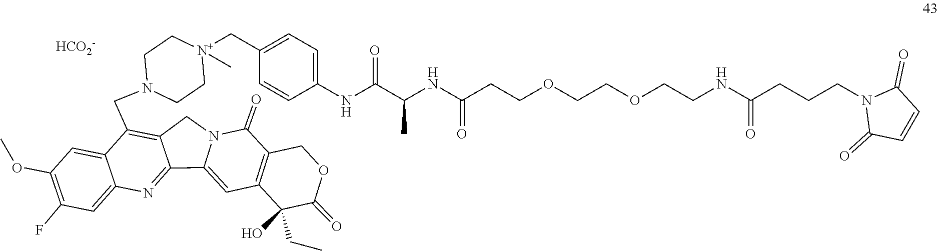 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)CCOCCOCCNC(=O)CCCN3C(=O)C=CC3=O)cc2)CC1 ; "HCO2-"]

Compound 40 (0.10 g, 0.13 mmol) in DMF (1 mL) and compound 12 (66 mg, 0.13 mmol) in DMF (2 mL) were mixed, and then N, N-diisopropylethylamine (90 μL, 0.52 mmol) was added. The reaction mixture was stirred at r.t. for 1 hour, concentrated, and purified by preparative HPLC (acetonitrile/water containing formic acid) to give compound 43 (50.9 mg, 39% yield). ESI-MS m/z: M+ calcd for C₅₂H₆₂N₈O₁₂: 1009.45. found 1009.45.

### Example 38. Synthesis of (S)-3-((tert-butoxycarbonyl)amino)-2-(2,5-dioxo-2,5-dihydro-1H-pyrrol-1-yl)propanoic acid (44)

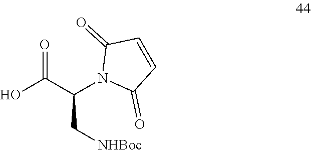 [SMILES: CC(C)(C)OC(=O)NC[C@@H](C(=O)O)N1C(=O)C=CC1=O]

To the solution of 2-amino-3-((tert-butoxycarbonyl)amino)propanoic acid (1 g, 4.90 mmol) in a saturated solution of NaHCO₃ (20 mL) was added methyl 2,5-dioxo-2,5-dihydro-1H-pyrrole-1-carboxylate (1.52 g, 9.80 mmol) in ice-water bath. The reaction was stirred for 30 min and then poured into a separatory funnel containing 100 mL of ethyl acetate and the organic phase was separated, washed with 50 mL of water and 50 mL of brine, dried over anhydrous Na₂SO₄, filtered and concentrated to give compound 4 (1.39 g, yield 72%).

### Example 39. Synthesis of (S)-perfluorophenyl 3-((tert-butoxycarbonyl)amino)-2-(2,5-dioxo-2,5-dihydro-1H-pyrrol-1-yl)propanoate (45)

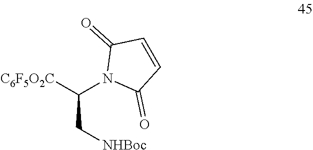

To a solution of compound 44 (0.10 g, 0.35 mmol) dissolved in dichloromethane (30 mL), were added pentafluorophenol (97 mg, 0.52 mmol) and 1-(3-dimethylaminopropyl)-3-ethylcarbodiimide hydrochloride (0.13 g, 0.7 mmol). The reaction was stirred at r.t. for 2 hours and diluted with dichloromethane (50 mL), washed with water (200 mL), dried over sodium sulfate, filtered, and concentrated to give compound 45 (0.16 g, 100% yield). ESI-MS m/z: [M+H]⁺ calcd for C₁₈H₁₅F₅N₂O₆: 451.09. found 451.09.

### Example 40. Synthesis of Compound 46

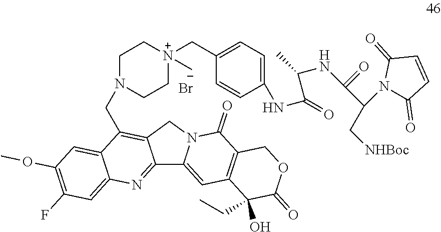

Compound 40 (0.05 g, 0.065 mmol) and compound 45 (45 mg, 0.10 mmol) were dissolved in DMF (3 mL), and then N, N-diisopropylethylamine (45 μL, 0.26 mmol) was added. The reaction was stirred at r.t. for 1 hour, concentrated and purified by preparative HPLC (acetonitrile/water containing formic acid) to yield compound 46 (35 mg, 52% yield). ESI-MS m/z: M+ calcd for C₄₉H₅₆FN₈O₁₁: 951.41. found 951.41.

### Example 41. Synthesis of 1-(4-((S)-2-((S)-3-amino-2-(2,5-dioxo-2,5-dihydro-1H-pyrrol-1-yl)propanamido)propanamido)benzyl)-4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium (47)

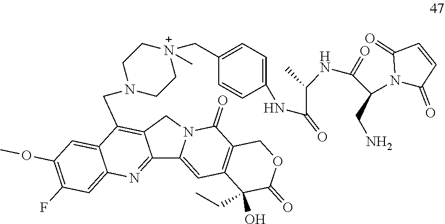 [SMILES: CC[C@@]1(O)C(=O)OCc2c1cc1n(c2=O)Cc2c-1nc1cc(F)c(OC)cc1c2CN1CC[N+](C)(Cc2ccc(NC(=O)[C@H](C)NC(=O)[C@H](CN)N3C(=O)C=CC3=O)cc2)CC1]

Compound 46 (35 mg, 0.03 mmol) was dissolved in dichloromethane (2 mL) and treated with trifluoroacetic acid (1 mL). After stirring at r.t. for 1 hour, the reaction mixture was concentrated, co-evaporated with dichloromethane twice and dried on a vacuum pump to give compound 47 (30.4 mg, 96% yield). ESI-MS m/z: M+ calcd for C₄₄H₄₈FN₈O₉: 851.35. found 851.35.

### Example 42. Synthesis of (S)-tert-butyl (1-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)piperidin-4-yl)carbamate (48)

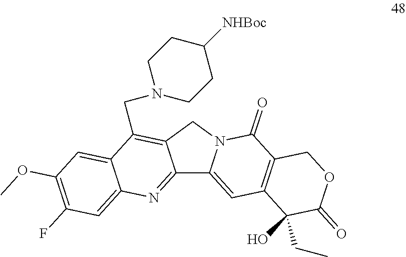 [SMILES: CC[C@@]1(O)C(=O)OCc2c1cc1n(c2=O)Cc2c-1nc1cc(F)c(OC)cc1c2CN1CCC(NC(=O)OC(C)(C)C)CC1]

Compound 26d (50 mg, 0.11 mmol) was dissolved in DMF (3 mL), and then tert-butyl piperidin-4-ylcarbamate (25 mg, 0.12 mmol) was added and stirred at r.t. for 5 hours. The mixture was concentrated and purified by preparative HPLC (acetonitrile/water containing formic acid) to yield compound 48 (30 mg, 45% yield). ESI-MS m/z: [M+H]⁺ calcd for C₃₂H₃₇FN₄O₇: 609.26; found 609.26.

### Example 43. Synthesis of(S)-11-((4-aminopiperidin-1-yl)methyl)-4-ethyl-8-fluoro-4-hydroxy-9-methoxy-1H-pyrano[3′,4′:6,7]indolizino[1,2-b]quinoline-3,14(4H,12H)-dione (49)

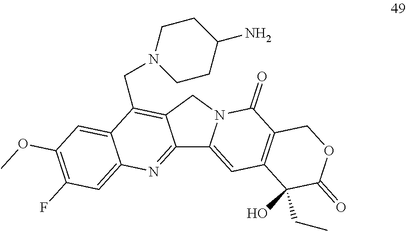 [SMILES: CC[C@@]1(O)C(=O)OCc2c1cc1n(c2=O)Cc2c-1nc1cc(F)c(OC)cc1c2CN1CCC(N)CC1]

Compound 48 (30 mg, 0.03 mmol) was dissolved in dichloromethane (2 mL) and treated with trifluoroacetic acid (2 mL). After stirring at r.t. for 1 hour, the mixture was concentrated, co-evaporated with dichloromethane twice and dried on a vacuum pump to give compound 49 (25.4 mg, 100% yield). ESI-MS m/z: [M+H]⁺ calcd for C₂H₃₀FN₄₅: 509.21. found 509.21.

### Example 44. Synthesis of (S)-2-(4-(2,5-dioxo-2,5-dihydro-1H-pyrrol-1-yl)butanamido)-N1-(4-((1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)piperidin-4-yl)amino)-4-oxobutyl)-N5-(2,5,8,11,14,17,20,23-octaoxapentacosan-25-yl)pentanediamide (50)

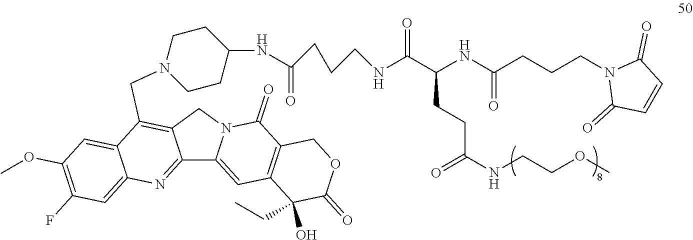

Compound 49 (25.4 mg, 0.05 mmol) was dissolved in DMF (2 mL), to which compound 7 (38.1 mg, 0.05 mmol) was added, followed by HATU (28.5 mg, 0.08 mmol) and triethylamine (14 μL, 0.1 mmol) in sequence, and the reaction was stirred at r.t. for 1 h, concentrated and purified by preparative HPLC (acetonitrile/water containing formic acid) to give compound 50 (14.4 mg, 23% yield). ESI-MS m/z: [M+H]⁺ calcd for C₆₁H₈₅FN₈O₁₉: 1253.59. found 1253.59.

### Example 45. Synthesis of tert-butyl bis(2-(2,2,2-trifluoroacetamido)ethyl)carbamate (51)

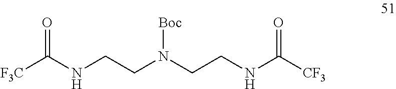 [SMILES: CC(C)(C)OC(=O)N(CCNC(=O)C(F)(F)F)CCNC(=O)C(F)(F)F]

To a solution of diethylenetriamine (6.18 g, 60 mmol) in dichloromethane (120 mL), was added dropwise a solution of ethyl trifluoroacetate (18.75 g, 132 mmol) in dichloromethane (60 mL) at 0° C. The solution was stirred for 30 minutes, and then warmed to r.t. and stirred for 1 hour. A solution of di-tert-butyl dicarbonate (28.78 g, 132 mmol) and triethylamine (13.33 g, 132 mmol) in dichloromethane (60 mL) was added dropwise at r.t. and stirred overnight. The reaction mixture was washed with saturated sodium carbonate (2×200 mL), water (2×200 mL), brine (200 mL), dried over sodium sulfate, filtered and concentrated. The residue was purified by silica gel column (petroleum ether/ethyl acetate) to give a white solid (17.4 g, 73.3% yield). ESI-MS m/z: [M+H]⁺ calcd for C₁₃H₁₉F₆N₃O₄: 396.30. found 396.28.

### Example 46. Synthesis of tert-butyl bis(2-aminoethyl)carbamate (52)

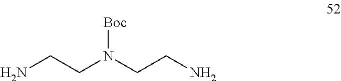 [SMILES: CC(C)(C)OC(=O)N(CCN)CCN]

Compound 51 (4.28 g, 10.8 mmol) was dissolved in MeOH (50 mL) and stirred with a solution of sodium hydroxide (5.42 g, 135 mmol) in water (50 mL) at r.t. for 3 hours. The reaction was concentrated, extracted with dichloromethane (3×100 mL), the organic phase was washed with brine (100 mL), dried with sodium sulfate, filtered and concentrated to give compound 3 (1.8 g, 82% yield). ESI-MS m/z: [M+H]⁺ calcd for C₉H₂₁N₃O₂ 204.28. found 204.12.

### Example 47. Synthesis of 4,4′-((((tert-butoxycarbonyl)azanediyl)bis(ethane-2,1-diyl))bis(azanediyl))bis(4-oxobutanoic acid) (53)

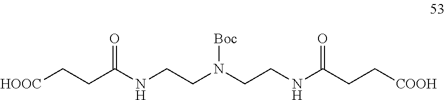 [SMILES: CC(C)(C)OC(=O)N(CCNC(=O)CCC(=O)O)CCNC(=O)CCC(=O)O]

Compound 52 (1.8 g, 8.8 mmol) was dissolved in dichloromethane (150 mL), to which succinic anhydride (2.2 g, 22.1 mmol) was added. After stirring at r.t. overnight, the reaction was concentrated and purified on silica gel column, eluting with dichloromethane/MeOH to yield compound 53 (2.99 g, 84% yield). ESI-MS m/z: [M+H]⁺ calcd for C₁₇H₂₉N₃O₈: 404.43. found 404.11.

### Example 48. Synthesis of bis((S)-4-ethyl-4-hydroxy-3,14-dioxo-3,4,12,14-tetrahydro-1H-pyrano[3′,4′:6,7]indolizino[1,2-b]quinolin-9-yl) 4,4′-((((tert-butoxycarbonyl)azanediyl)bis(ethane-2,1-diyl))bis(azanediyl))bis(4-oxobutanoate) (54)

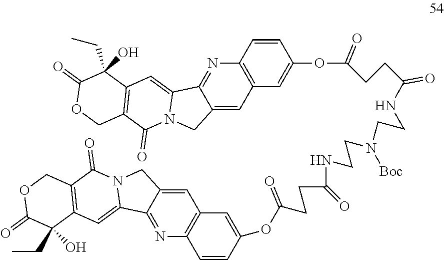 [SMILES: CC[C@@]1(O)C(=O)OCc2c1cc1n(c2=O)Cc2cc3cc(OC(=O)CCC(=O)NCCN(CCNC(=O)CCC(=O)Oc4ccc5nc6c(cc5c4)Cn4c-6cc5c(c4=O)COC(=O)[C@]5(O)CC)C(=O)OC(C)(C)C)ccc3nc2-1]

To a solution of compound 53 (853 mg, 2.1 mmol) and compound 5 (1.71 g, 4.7 mmol) in DMF (100 mL), triethylamine (948 mg, 9.4 mmol) and HATU (1.79 g, 4.7 mmol) were added in sequence. The resulting mixture was stirred overnight, and then concentrated, purified by silica gel column (dichloromethane/MeOH) to give compound 54 (2.84 g, 100% yield). ESI-MS m/z: [M+H]⁺ calcd for C₈₇H₅₇N₇O₁₆: 1097.10. found 1097.65.

### Example 49. Synthesis of bis((S)-4-ethyl-4-hydroxy-3,14-dioxo-3,4,12,14-tetrahydro-1H-pyrano[3′,4′:6,7]indolizino[1,2-b]quinolin-9-yl) 4,4′-((azanediylbis(ethane-2,1-diyl))bis(azanediyl))bis(4-oxobutanoate) (55)

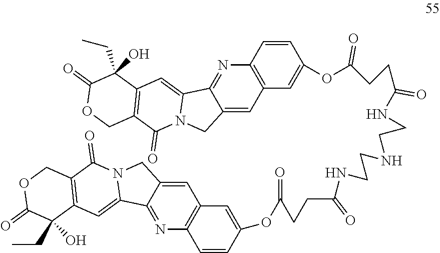 [SMILES: CC[C@@]1(O)C(=O)OCc2c1cc1n(c2=O)Cc2cc3cc(OC(=O)CCC(=O)NCCNCCNC(=O)CCC(=O)Oc4ccc5nc6c(cc5c4)Cn4c-6cc5c(c4=O)COC(=O)[C@]5(O)CC)ccc3nc2-1]

Compound 54 (2.84 g, 2.1 mmol) was dissolved in dichloromethane (40 mL), and trifluoroacetic acid (20 mL) was added. The reaction was stirred at r.t. for 1 hour and then concentrated to give compound 55 (3.3 g, 100% yield). ESI-MS m/z: [M+H]⁺ calcd for C₈₂H₄₉N₇O₁₄: 996.98. found 996.60.

### Example 50. Synthesis of (S)-(S)-4-ethyl-4-hydroxy-3,14-dioxo-3,4,12,14-tetrahydro-1H-pyrano[3′,4′:6,7]indolizino[1,2-b]quinolin-9-yl 30-(4-(2,5-dioxo-2,5-dihydro-1H-pyrrol-1-yl)butanamido)-37-(2-(4-(((S)-4-ethyl-4-hydroxy-3,14-dioxo-3,4,12,14-tetrahydro-1H-pyrano[3′,4′:6,7]indolizino[1,2-b]quinolin-9-yl)oxy)-4-oxobutanamido)ethyl)-27,31,36,41-tetraoxo-2,5,8,11,14,17,20,23-octaoxa-26,32,37,40-tetraazatetratetracontan-44-oate (56)

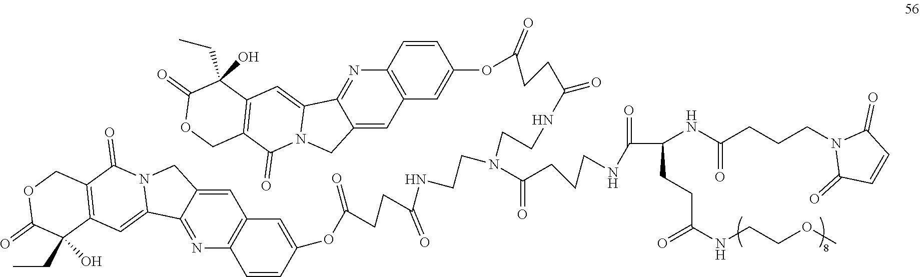

To a solution of compound 55 (614 mg, 0.60 mmol) and compound 53 (470 mg, 0.60 mmol) in DMF (20 mL), triethylamine (249 mg, 2.5 mmol) and HATU (234 mg, 0.60 mmol) were added in sequence. The resulting mixture was stirred for 40 minutes, and then concentrated, purified by silica gel column (MeOH/dichloromethane) to give compound 56 (46 mg, 5% yield). ESI-MS m/z: [M+H]⁺ calcd for C₈₆H₁₀₅N₁₁O₂₈: 17410.81. found 1742.01.

### Example 51. Synthesis of (S)-4-ethyl-4-hydroxy-9-methoxy-1H-pyrano[3′,4′:6,7]indolizino[1,2-b]quinoline-3,14(4H,12H)-dione (57)

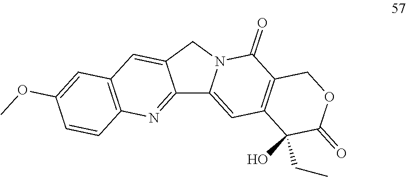 [SMILES: CC[C@@]1(O)C(=O)OCc2c1cc1n(c2=O)Cc2cc3cc(OC)ccc3nc2-1]

10-hydroxycamptothecin (2.5 g, 6.86 mmol) was dissolved in DMF (150 mL), to which potassium carbonate (1.90 g, 13.72 mmol) and methyl iodide (1.17 g, 8.23 mmol) were added, and the reaction was stirred at r.t. overnight. A mixed solvent of petroleum ether (150 mL) and ethyl acetate (150 mL) was added to the reaction mixture and stirred. A yellow solid was precipitated out and collected by filtration, then dispersed in water (20 mL). 1N hydrochloric acid was added dropwise until pH 7, and the mixture was filtered again to give compound 57 (1.0 g, 38% yield). ESI-MS m/z: [M+H]⁺ calcd for C₂₁H₁₈N₂O₅ 379.38. found 379.05.

### Example 52. Synthesis of bis((S)-4-ethyl-9-methoxy-3,14-dioxo-3,4,12,14-tetrahydro-1H-pyrano[3′,4′:6,7]indolizino[1,2-b]quinolin-4-yl) (((tert-butoxycarbonyl)azanediyl)bis(ethane-2,1-diyl))dicarbamate (59)

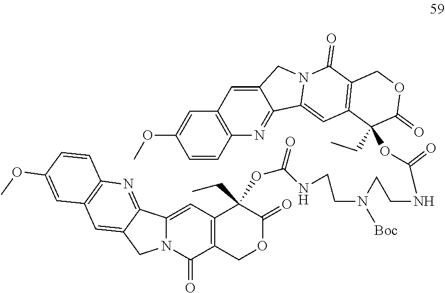 [SMILES: CC[C@@]1(OC(=O)NCCN(CCNC(=O)O[C@]2(CC)C(=O)OCc3c2cc2n(c3=O)Cc3cc4cc(OC)ccc4nc3-2)C(=O)OC(C)(C)C)C(=O)OCc2c1cc1n(c2=O)Cc2cc3cc(OC)ccc3nc2-1]

Compound 57 (350 mg, 0.9 mmol), 4-dimethylaminopyridine (339 mg, 2.8 mmol) and triphosgene (93 mg, 0.34 mmol) were crushed and mixed evenly under N₂, anhydrous dichloromethane (8 mL) was then added dropwise and stirred for 10 minutes. A solution of compound 52 (64 mg, 0.34 mmol) dissolved in anhydrous dichloromethane (4 mL) was added to the mixture, followed by triethylamine (93 mg, 0.9 mmol). After stirring for 15 minutes, the solution was concentrated, and purified by silica gel column (MeOH/dichloromethane) to give compound 59 (200 mg, 22% yield). ESI-MS m/z: [M+H]⁺ calcd for C₈₃H₅₃N₇O₁₄: 1013.03; found 1013.26.

### Example 53. Synthesis of bis((S)-4-ethyl-9-methoxy-3,14-dioxo-3,4,12,14-tetrahydro-1H-pyrano[3′,4′:6,7]indolizino[1,2-b]quinolin-4-yl) (azanediylbis(ethane-2,1-diyl))dicarbamate (60)

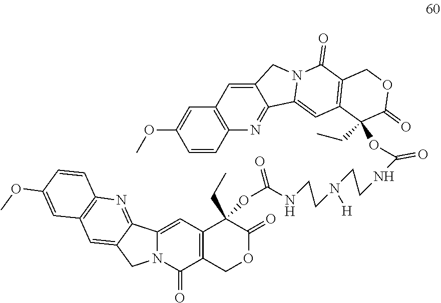 [SMILES: CC[C@@]1(OC(=O)NCCNCCNC(=O)O[C@]2(CC)C(=O)OCc3c2cc2n(c3=O)Cc3cc4cc(OC)ccc4nc3-2)C(=O)OCc2c1cc1n(c2=O)Cc2cc3cc(OC)ccc3nc2-1]

Compound 59 (200 mg, 0.2 mmol) was dissolved in dichloromethane (10 mL), and treated with trifluoroacetic acid (5 mL) for 4 hours. Concentration of the reaction mixture gave compound 60 (0.43 g, 100% yield). ESI-MS m/z: [M+H]⁺ calcd for C₄₈H₄₅N₇O₁₂: 912.91. found 912.62.

### Example 54. Synthesis of bis((S)-4-ethyl-9-methoxy-3,14-dioxo-3,4,12,14-tetrahydro-1H-pyrano[3′,4′:6,7]indolizino[1,2-b]quinolin-4-yl) (((4-(2,5-dioxo-2,5-dihydro-1H-pyrrol-1-yl)butanoyl)azanediyl)bis(ethane-2,1-diyl))dicarbamate (61)

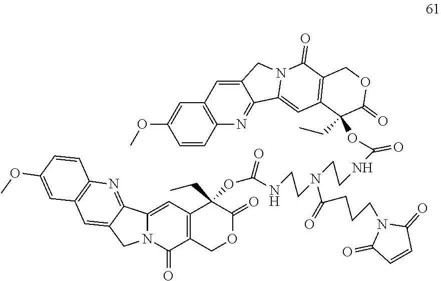 [SMILES: CC[C@@]1(OC(=O)NCCN(CCNC(=O)O[C@]2(CC)C(=O)OCc3c2cc2n(c3=O)Cc3cc4cc(OC)ccc4nc3-2)C(=O)CCCN2C(=O)C=CC2=O)C(=O)OCc2c1cc1n(c2=O)Cc2cc3cc(OC)ccc3nc2-1]

To a solution of compound 59 (249 mg, 0.27 mmol) and compound 7 (60 mg, 0.32 mmol) in dichloromethane (10 mL), were added triethylamine (112 μL, 0.81 mmol) and HATU (104 mg, 0.27 mmol). The reaction was stirred for 40 minutes, and then washed with water (20 mL). The organic phase was concentrated and purified by preparative HPLC (acetonitrile/water containing formic acid) to give compound 61 (50 mg). ESI-MS m/z: [M+H]⁺ calcd for C₈₆H₅₂N₈O₁₅ 1078.06. found 1078.77.

### Example 55. Synthesis of (S)-N,N′-(((((2S,20S)-11-(tert-butoxycarbonyl)-2,20-dimethyl-4,7,15,18-tetraoxo-3,8,11,14,19-pentaazahenicosane-1,21-dioyl)bis(azanediyl))bis(4,1-phenylene))bis(methylene))bis(1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium) (62)

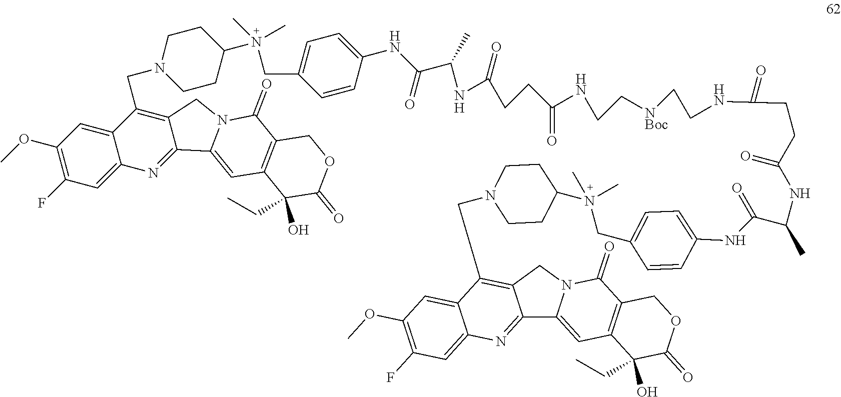

Compound 40 (96 mg, 0.132 mmol) and compound 53 (26 mg, 0.066 mmol) were dissolved in DMF (3 mL), and cooled to 0° C. HATU (50 mg, 0.132 mmol)) and N, N-diisopropylethylamine (46 μL, 0.264 mmol) were added, and the reaction was stirred at 0° C. for 30 minutes after addition was completed. The crude reaction mixture was purified directly on preparative HPLC (acetonitrile/water containing formic acid) (acetonitrile/water with 0.1% formic acid) to yield compound 62 (80 mg, 67% yield). ESI-MS m/z: [M]²⁺ calcd for C₉₁H₁₀₉F₂N₁₅O₁₈: 868.90. found 868.90.

### Example 56. Synthesis of (S)-N,N′-(((((2S,20S)-2,20-dimethyl-4,7,15,18-tetraoxo-3,8,11,14,19-pentaazahenicosane-1,21-dioyl)bis(azanediyl))bis(4,1-phenylene))bis(methylene))bis(1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium) (63)

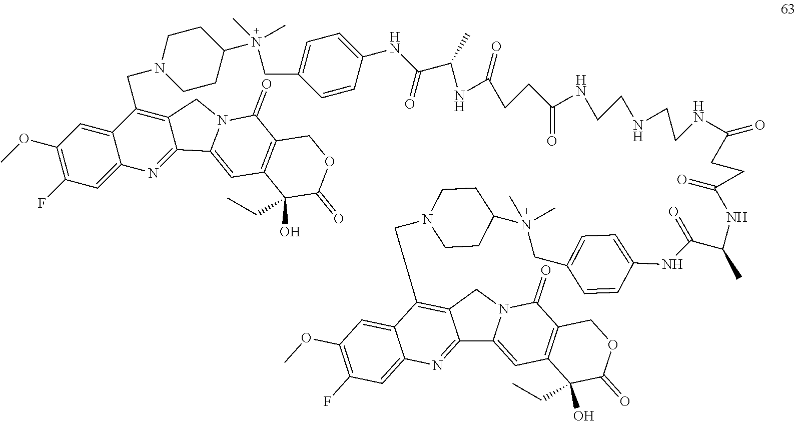

Compound 62 (80 mg, 0.043 mmol) was dissolved in a mixture of dichloromethane and trifluoroacetic acid (3 mL/1 mL), and stirred at r.t. for 30 minutes. Concentration of the reaction mixture afforded compound 63 (100% yield). ESI-MS m/z: [M]²⁺ calcd for C₈₆H₁₀₁F₂N₁₅O₁₆: 818.87. found 818.87.

### Example 57. Synthesis of (S)-N,N′-(((((2S,20S)-11-((S)-30-(4-(2,5-dioxo-2,5-dihydro-1H-pyrrol-1-yl)butanamido)-27,31-dioxo-2,5,8,11,14,17,20,23-octaoxa-26,32-diazahexatriacontan-36-oyl)-2,20-dimethyl-4,7,15,18-tetraoxo-3,8,11,14,19-pentaazahenicosane-1,21-dioyl)bis(azanediyl))bis(4,1-phenylene))bis(methylene))bis(1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium) (64)

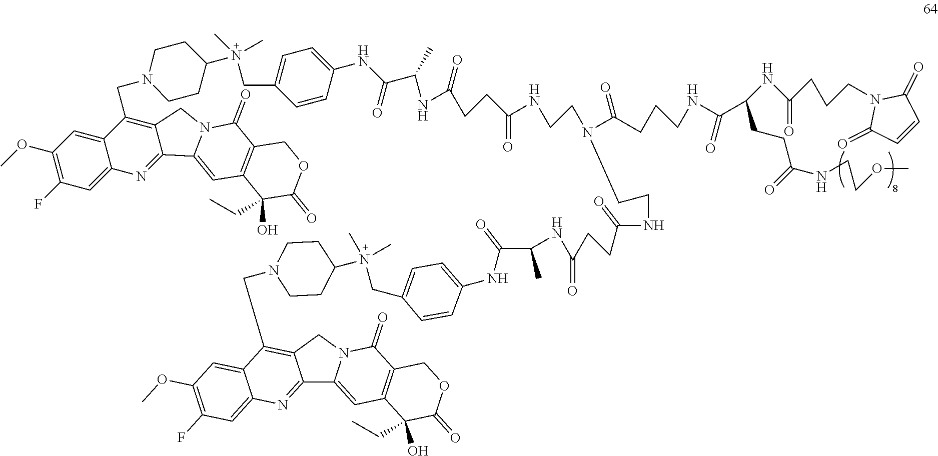

To a solution of compound 63 (74 mg, 0.043 mmol) and compound 7 (39 mg, 0.0516 mmol) in DMF (3 mL), N, N-diisopropylethylamine (15 μL, 0.086 mmol) was added at 0° C. The reaction was warmed to r.t. and stirred for 2 hours. After concentration, the residue was purified by preparative HPLC (acetonitrile/water containing formic acid) to yield compound 64 (12 mg). ESI-MS m/z: [M+]²⁺ calcd for C₁₂₀H₁₅₇F₂N₁₉O₃₀: 1191.06. found 1191.06.

### Example 58. Synthesis of (S)-4-ethyl-8-fluoro-4,9-dihydroxy-11-methyl-1H-pyrano[3′,4′:6,7]indolizino[1,2-b]quinoline-3,14(4H,12H)-dione (103)

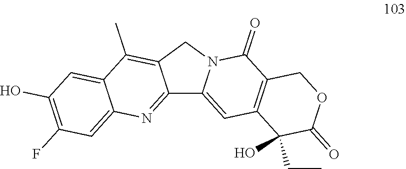 [SMILES: CC[C@@]1(O)C(=O)OCc2c1cc1n(c2=O)Cc2c-1nc1cc(F)c(O)cc1c2C]

1-(2-amino-4-fluoro-5-hydroxyphenyl)ethanone (0.41 g, 2.5 mmol) and compound 25 (0.62 g, 2.5 mmol) were dissolved in anhydrous toluene (40 mL), and p-toluenesulfonic acid (46 mg, 0.25 mmol) was added. The suspension was heated at reflux for 3 days and allowed to cool to r.t. After removal of the solvent, the residue was purified by column chromatography to give compound 103 (0.69 g, 73% yield) as a gray powdery solid. ESI-MS m/z: [M+H]⁺ calcd for C₂₁H₁₇FN₂O₅: 397.11. found 397.16.

### Example 59. Synthesis of (S)-9-(2-bromoethoxy)-4-ethyl-8-fluoro-4-hydroxy-11-methyl-1H-pyrano[3′,4′:6,7]indolizino[1,2-b]quinoline-3,14(4H,12H)-dione (104)

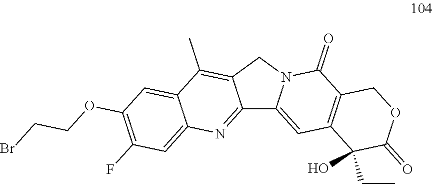 [SMILES: CC[C@@]1(O)C(=O)OCc2c1cc1n(c2=O)Cc2c-1nc1cc(F)c(OCCBr)cc1c2C]

A mixture of compound 103 (0.69 g, 1.74 mmol), anhydrous 1,2-dibromoethane (6.4 g, 34.8 mmol), and anhydrous K₂CO₃ (1.2 g, 8.7 mmol) in anhydrous DMF (10 mL) was mechanically stirred at 80° C. for 16 h. The reaction mixture was filtered through a pad of Celite, and the filtered residue was washed well with DMF. The combined filtrate and washings were evaporated to dryness in vacuo to afford a dark residue. The residue was purified by column chromatography (0-5% MeOH/dichloromethane) to afford compound 104 (0.74 g, 85%). ESI-MS m/z: [M+H]⁺ calcd for C₂₃H₂₀BrFN₂O₅: 503.05. found 503.05.

### Example 60. Synthesis of (S)-9-(2-bromoethoxy)-4-ethyl-8-fluoro-4-hydroxy-11-methyl-10-nitro-1H-pyrano[3′,4′:6,7]indolizino[1,2-b]quinoline-3,14(4H,12H)-dione (105)

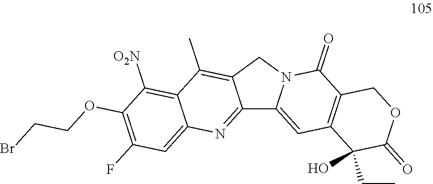 [SMILES: CC[C@@]1(O)C(=O)OCc2c1cc1n(c2=O)Cc2c-1nc1cc(F)c(OCCBr)c([N+](=O)[O-])c1c2C]

To a stirred concentrated H₂SO₄ (1 mL) at 0° C. was added compound 104 (0.74 g, 1.47 mmol) slowly, and the resulting clear solution was cooled to −10° C. A mixture of concentrated H₂SO₄ (0.5 mL) and fuming HNO₃ (0.5 mL), pre-cooled to −10° C., was added dropwise to the cooled reaction mixture at −10° C. The reaction mixture was allowed to warm to 0° C., stirred for an additional 1 h, and then poured slowly onto the ice chips. The yellow precipitate was filtered and washed with H₂O, cold EtOH, and Et₂O. The aqueous filtrate was filtered again through a pad of Celite, and the Celite filter cake was extracted with 30% MeOH/CH₂Cl₂ (50 mL). Evaporation of the organic solvent afforded an additional yellow solid. Trituration of the combined yellow solid with EtOH afforded compound 105 (0.74 g, 92%). ESI-MS m/z [M+H]⁺: calcd for C₂₃H₁₉BrFN₃O₇: 548.04. found 548.14.

### Example 61. Synthesis of (S)-10-amino-9-(2-bromoethoxy)-4-ethyl-8-fluoro-4-hydroxy-11-methyl-1H-pyrano[3′,4′:6,7]indolizino[1,2-b]quinoline-3,14(4H,12H)-dione (106)

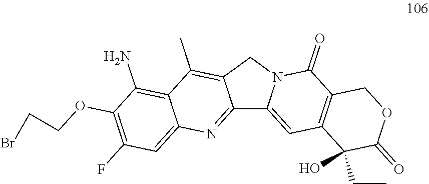 [SMILES: CC[C@@]1(O)C(=O)OCc2c1cc1n(c2=O)Cc2c-1nc1cc(F)c(OCCBr)c(N)c1c2C]

To a stirred concentrated HCl solution (18 mL) at 0° C. was added compound 105 (0.50 g, 0.91 mmol) in small portions, and the resulting clear solution was cooled to −10° C. after 15 min. To the reaction mixture was added SnCl₂ (0.86 g, 4.55 mmol) in small portions and the reaction mixture was allowed to warm to r. t., stirred for 1.5 h, and then poured slowly onto the ice chips. The precipitate was filtered and washed with EtOH and Et₂O, and the aqueous filtrate was extracted with 10% MeOH/CH₂Cl₂. The organic solution was combined with the filtered precipitate dissolved in 30% MeOH/CH₂Cl₂, and then passed through a short SiO₂ pad and eluted with 30% MeOH/CH₂Cl₂. The organic solvent was removed to afford compound 106 (0.44 g, 94%), which was used in the next step without further purification.

### Example 62. Synthesis of (S)-9-ethyl-5-fluoro-9-hydroxy-16-methyl-2,3,12,15-tetrahydro-[1,4]oxazino[3,2-f]pyrano[3′,4′:6,7]indolizino[1,2-b]quinoline-10,13(1H,9H)-dione (107)

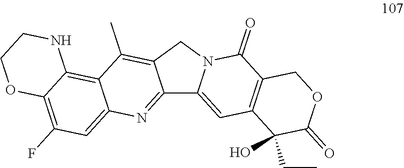 [SMILES: CC[C@@]1(O)C(=O)OCc2c1cc1n(c2=O)Cc2c-1nc1cc(F)c3c(c1c2C)NCCO3]

A solution of compound 106 (0.44 g, 0.85 mmol) in DMSO (4 mL) and NaHCO₃ (0.10 g, 1.19 mmol) was stirred at 70° C. for 4 h, and diluted with HCl (0.1 M, 8 ml) and H₂O (40 mL). The precipitated solid was filtered, dissolved in a small volume of 10% MeOH/CH₂Cl₂, and purified by column chromatography using (1:20-1:6) MeOH/CH₂Cl₂ as eluent to afford compound 107 (0.24 g, 66%). ESI-MS m/z: [M+H]⁺ calcd for C₂₃H₂₀FN₃O₅: 438.14. found 438.14.

### Example 63. Synthesis of (S)-tert-butyl (2-(9-ethyl-5-fluoro-9-hydroxy-16-methyl-10,13-dioxo-2,3,9,10-tetrahydro-[1,4]oxazino[3,2-f]pyrano[3′,4′:6,7]indolizino[1,2-b]quinolin-1(12H,13H,15H)-yl)ethyl)carbamate (108)

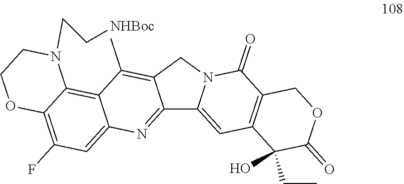 [SMILES: CC[C@@]1(O)C(=O)OCc2c1cc1n(c2=O)Cc2c-1nc1cc(F)c3c(c1c2C)N(CCNC(=O)OC(C)(C)C)CCO3]

To a stirred solution of compound 107 (0.20 g, 0.456 mmol) in anhydrous DMF (2 mL) were added NaI (0.68 g, 4.56 mmol) and tert-butyl (2-chloroethyl)carbamate (0.82 g, 4.56 mmol), and the mixture was heated at 120° C. for 18 h. The reaction mixture was cooled to r. t., evaporated to dryness in vacuo, and purified by column chromatography (0-5% MeOH/CH₂Cl₂) to afford compound 108 (0.19 g, 75%). ESI-MS m/z: [M+H]⁺ calcd for C₃₀H₃₃FN₄O₇: 581.23; found 581.40.

### Example 64. Synthesis of (S)-1-(2-aminoethyl)-9-ethyl-5-fluoro-9-hydroxy-16-methyl-2,3,12,15-tetrahydro-[1,4]oxazino[3,2-f]pyrano[3′,4′:6,7]indolizino[1,2-b]quinoline-10,13(1H,9H)-dione (109)

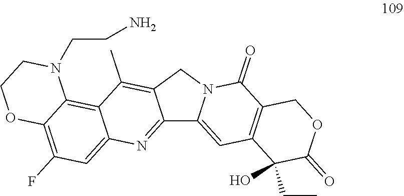 [SMILES: CC[C@@]1(O)C(=O)OCc2c1cc1n(c2=O)Cc2c-1nc1cc(F)c3c(c1c2C)N(CCN)CCO3]

To a solution of compound 108 (0.19 g, 0.327 mmol) in dichloromethane (5 mL) was added TFA (2.5 mL) and the reaction was stirred at r.t. for 30 min. The reaction mixture was concentrated, co-evaporated with dichloromethane for three times to afford compound 109, which was used in the next step without further purification.

### Example 65. Synthesis of Compound 110

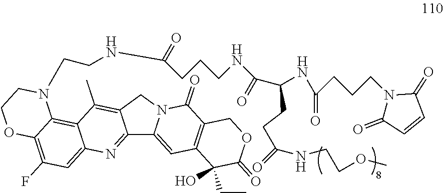

Compound 109 from the previous step and compound 7 (0.45 g, 0.49 mmol) were dissolved in DMF (5 mL), cooled to about 0° C., and then N, N-diisopropylethylamine (172 μL, 0.98 mmol,) was added. The reaction was warmed to r.t. and stirred for 2 hours, concentrated, and purified by preparative HPLC (acetonitrile/water containing formic acid) to give compound 110 (359 mg, 60% yield). ESI-MS m/z: [M+H]⁺ calcd for C₅₉H₈₁FN₈O₁₉: 1224.56. found 1224.78.

### Example 66. Synthesis of di-tert-butyl 4,4′-(((2R,3S)-2,3-bis(((benzyloxy)carbonyl)-amino)succinyl)bis(azanediyl))dibutanoate (172)

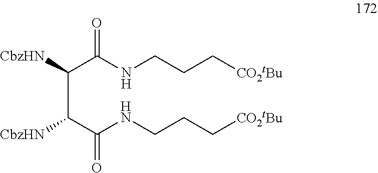 [SMILES: CC(C)(C)OC(=O)CCCNC(=O)[C@H](NC(=O)OCc1ccccc1)[C@@H](NC(=O)OCc1ccccc1)C(=O)NCCCC(=O)OC(C)(C)C]

To a solution of compound 171 (4.25 g, 10.21 mmol) in DMA (70 mL) were added tert-butyl 4-aminobutanoate (3.25 g, 20.42 mmol), DMAP (4.47 g, 36.65 mmol) and EDC HCl (7.00 g, 36.65 mmol). The mixture was stirred overnight, concentrated and purified on SiO₂ column, eluted with EtOAc/CH₂Cl₂ (1:10) to afford compound 172 (6.50 g, 91% yield). ESI-MS m/z: [M+H]⁺ calcd for C₃₆H₅₀N₄O₁₀ 699.35. found 699.55.

### Example 67. Synthesis of di-tert-butyl 4,4′-(((2R,3S)-2,3-diaminosuccinyl)-bis(azanediyl))dibutanoate (173)

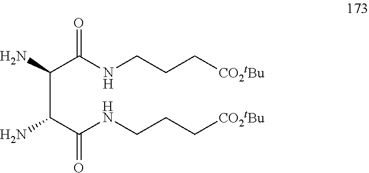 [SMILES: CC(C)(C)OC(=O)CCCNC(=O)[C@H](N)[C@@H](N)C(=O)NCCCC(=O)OC(C)(C)C]

To a solution of compound 172 (2.50 g, 3.58 mmol) in MeOH (100 mL) was added 10% Pd/C (0.30 g, 50% wet), and the mixture was stirred under hydrogen atmosphere at r.t. for 18 h. Then the Pd/C catalyst was removed by filtration over Celite and the filter cake was washed with MeOH (˜70 mL). The filtrate was concentrated to afford compound 173 as yellow foam which was used in the next step without further purification (1.54 g, 100% yield). ESI-MS m/z: [M+H]⁺ calcd for C₂₀H₃₈N₂O₆: 431.28. found 431.50.

### Example 68. Synthesis of di-tert-butyl 4,4′-(((2R,3R)-2,3-bis(4-(2,5-dioxo-2,5-dihydro-1H-pyrrol-1-yl)butanamido)succinyl)bis(azanediyl))dibutanoate (174)

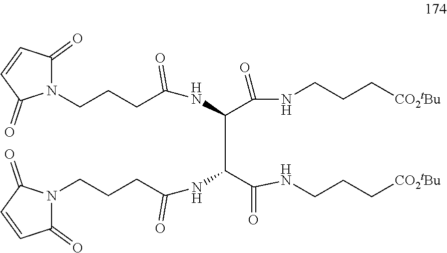 [SMILES: CC(C)(C)OC(=O)CCCNC(=O)[C@H](NC(=O)CCCN1C(=O)C=CC1=O)[C@@H](NC(=O)CCCN1C(=O)C=CC1=O)C(=O)NCCCC(=O)OC(C)(C)C]

To a solution of 4-(2,5-dioxo-2,5-dihydro-1H-pyrrol-1-yl)butanoic acid (1.35 g, 7.39 mmol) and compound 173 (1.54 g, ˜3.58 mmol) in DMF (60 mL) were added N, N-diisopropylethylamine (2.2 mL, 12.56 mmol) and HATU (4.77 g, 12.56 mmol). The mixture was stirred overnight, concentrated and purified on SiO₂ column, eluted with EtOAc/CH₂Cl₂ (1:10) to afford the title compound (2.35 g, 86% yield). ESI-MS m/z calcd for [M+H]⁺: C₃₄H₅₂N₆O₁₂ 761.36, found 761.36.

### Example 69. Synthesis of 4,4′-(((2R,3R)-2,3-bis(4-(2,5-dioxo-2,5-dihydro-1H-pyrrol-1-yl)butanamido)succinyl)bis(azanediyl))dibutanoic acid (175)

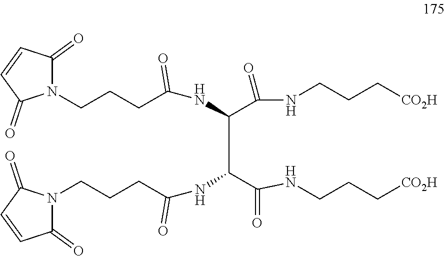 [SMILES: O=C(O)CCCNC(=O)[C@H](NC(=O)CCCN1C(=O)C=CC1=O)[C@@H](NC(=O)CCCN1C(=O)C=CC1=O)C(=O)NCCCC(=O)O]

To a stirred solution of compound 174 (2.30 g, 3.02 mmol) in dichloromethane (20 mL) was added TFA (10 mL). The mixture was stirred for 30 min, diluted with toluene (20 mL), concentrated to afford the title compound (1.69 g, 86% yield). ESI-MS m/z [M+H]⁺: calcd for C₂₈H₃₆N₆O₁₂ 649.24, found 649.30.

### Example 70. Synthesis of bis(2,5-dioxopyrrolidin-1-yl) 4,4′-(((2R,3R)-2,3-bis(4-(2,5-dioxo-2,5-dihydro-1H-pyrrol-1-yl)butanamido)succinyl)bis(azanediyl))dibutanoate (176)

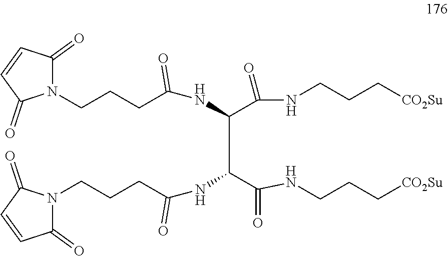

To a solution of compound 175 (1.10 g, 1.69 mmol) in DMA (30 mL) were added N-hydroxysuccinimide (1-hydroxypyrrolidine-2,5-dione) (0.58 g, 5.08 mmol) and EDC HCl (1.25 g, 6.54 mmol). The mixture was stirred overnight, concentrated and purified on SiO₂ column, eluted with EtOAc/CH₂Cl₂ (1:10) to afford the title compound (1.30 g, 91% yield). ESI-MS m/z [M+H]⁺: calcd for C₃₆H₄₂N₈O₁₆ 843.27, found 843.50.

### Example 71. Synthesis of (S)-N,N′-(((((2S,10S,11S,19S)-10,11-bis(4-(2,5-dioxo-2,5-dihydro-1H-pyrrol-1-yl)butanamido)-2,19-dimethyl-4,9,12,17-tetraoxo-3,8,13,18-tetraazaicosane-1,20-dioyl)bis(azanediyl))bis(4,1-phenylene))bis(methylene))bis(1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium) (177)

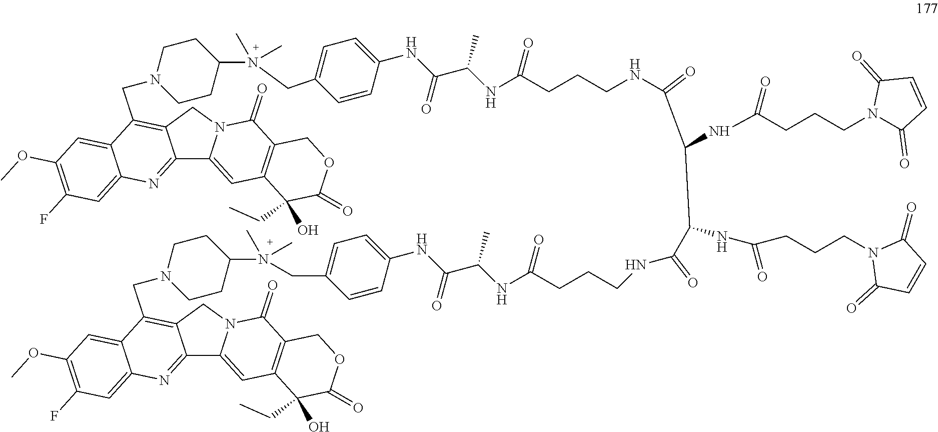

Compound 28 (94 mg, 0.12 mmol) and compound 176 (55 mg, 0.066 mmol) were dissolved in DMA (5 mL), cooled to about 0° C., and then N, N-diisopropylethylamine (84 μL, 0.48 mmol) was added. The reaction was warmed to r.t. and stirred for 2 hours, concentrated, and purified by preparative HPLC (acetonitrile/water containing formic acid) to give compound 177 (23 mg, 19% yield). ESI-MS m/z: M²⁺ calcd for C₁₀₆H₁₂₄F₂N₁₈O₂₂: 1019.46. found 1019.50.

### Example 72. Synthesis of 3-oxo-1-phenyl-2,7,10,13,16-pentaoxa-4-azanonadecan-19-oic acid (179)

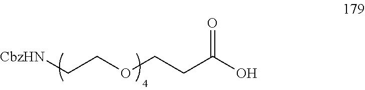

In a 500 mL flask, H₂N-PEG₄-CH₂CH₂CO₂H (3.0 g, 11.3 mmol, 1.0 eq.) and K₂CO₃ (4.7 g, 33.93 mmol, 3.0 eq.) were dissolved in 50 mL of water, and cooled over an ice water bath. CbzCl (2.50 g, 14.7 mmol, 1.3 eq.) in 50 mL of THF was added dropwise. The reaction was warmed to r.t. and stirred overnight. The reaction mixture was adjusted to pH 4-5 with 1N KHSO₄ and extracted with dichloromethane (200 mL×1, 100 mL×3), washed with water (500 mL), and brine (500 mL), dried over anhydrous sodium sulfate, and concentrated. The residue was dissolved in a small amount of dichloromethane and then loaded on a silica gel column, eluted with 2-4% MeOH/dichloromethane, and the fractions were combined and concentrated to give 3.8 g of colorless oil (yield 84%). ESI-MS m/z [M+H]⁺: calcd for C₁₉H₂₉NO₈ 400.2, found: 400.2.

### Example 73. Synthesis of 2,5-dioxopyrrolidin-1-yl 3-oxo-1-phenyl-2,7,10,13,16-pentaoxa-4-azanonadecan-19-oate (180)

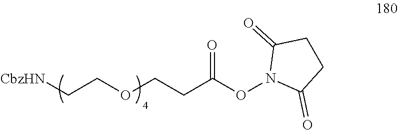

To a solution of CbzHN-PEG₄-CH₂CH₂CO₂H (3.8 g, 9.5 mmol, 1.0 eq.) in 50 mL of dry dichloromethane, N-hydroxysuccinimide (1.3 g, 11.4 mmol, 1.2 eq.) and EDC·HCl (9.1 g, 47.5 mmol, 5.0 eq.) were added. The reaction was stirred at r.t. overnight and then washed with water (50 mL×2), brine (100 mL×1), dried over anhydrous sodium sulfate, and concentrated. The crude product was used directly in the next step. ESI-MS m/z [M+H]⁺: calcd for C₂₃H₃₂N₂O₁₀ 497.2, found: 497.2.

### Example 74. Synthesis of 3,19-dioxo-1-phenyl-2,7,10,13,16,23,26,29,32-nonaoxa-4,20-diazapentatriacontan-35-oic acid (181)

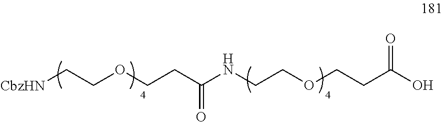

In a 300 mL flask, H₂N-PEG₄-CH₂CH₂CO₂H (2.6 g, 9.5 mmol, 1.0 eq.) and K₂CO₃ (3.9 g, 28.5 mmol, 3.0 eq.) were dissolved in 40 mL of water, cooled over an ice water bath, and the above crude N-hydroxysuccinimide ester solution (3.8 g, 9.5 mmol) in 40 mL of THF was added dropwise, and the mixture was warmed to r.t. and stirred overnight. The reaction mixture was adjusted to pH 4-5 using 1N KHSO₄, extracted with dichloromethane (150 mL×1, 100 mL×2), washed with water (200 mL), and brine (200 mL), dried over anhydrous sodium sulfate, and concentrated. The residue was dissolved in small amount of dichloromethane, and the loaded on a silica gel column, eluted with 4-6% MeOH/dichloromethane to give a colorless oil (4.91 g, 80% yield). ESI-MS m/z [M+H]⁺: calcd for C₃₀H₅₀N₂O₁₃ 646.3, found: 646.3.

### Example 75. Synthesis of tert-butyl 3,19,35-trioxo-1-phenyl-2,7,10,13,16,23,26,29,32,39,42,45,48-tridecaoxa-4,20,36-triazahenpentacontan-51-oate (182)

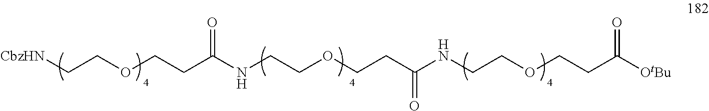

H₂N-PEG₄-CH₂CH₂CO₂^{t}Bu (0.48 g, 1.5 mmol, 1.0 eq.) was dissolved in 3 mL of DMF, cooled over ice/water bath, N, N-diisopropylethylamine (DIPEA) (0.78 g, 6.0 mmol, 4.0 eq.) was added dropwise, and followed by a solution of compound 181 (0.97 g, 1.5 mmol, 1.0 eq.) in 7 mL of DMF and HATU (1.72 g, 4.5 mmol, 3.0 eq.). The reaction was stirred over the ice bath for 2 hours, and diluted with 100 mL of water, extracted with dichloromethane (100 mL×3), washed with 1N KHSO₄ (200 mL), saturated sodium bicarbonate (200 mL), and brine (200 mL), dried over anhydrous sodium sulfate, and concentrated. The residue was dissolved in a small amount of dichloromethane, loaded on a silica gel column, and eluted 0-5% MeOH/dichloromethane.

Fractions were combined and concentrated to give 1.22 g of light yellow oil (86% yield). ESI-MS m/z [M+H]⁺: calcd for C₄₅H₇₉N₃O₁₈ 950.5, found: 950.5.

### Example 76. Synthesis of tert-butyl 1-amino-15,31-dioxo-3,6,9,12,19,22,25,28,35,38,41,44-dodecaoxa-16,32-diazaheptatetracontan-47-oate (183)

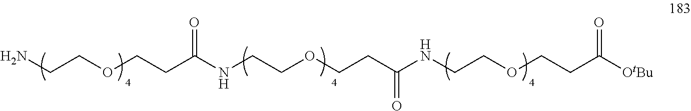

A solution of compound 182 (1.22 g, 1.28 mmol) in dichloromethane (5 mL) was stirred with Pd/C (5% wet, 500 mg) under 1 atm H₂ for 2 h. The reaction was then filtered over Celite and the filter cake was washed with MeOH. The filtrate and washings were combined and concentrated to give a light yellow oil (1.04 g, 100% yield). ESI-MS m/z [M+H]⁺: calcd for C₃₇H₇₃N₃O₁₆ 816.5, found: 816.5.

### Example 77. Synthesis of (50R,51R)-di-tert-butyl 50,51-bis(((benzyloxy)carbonyl)amino)-17,33,49,52,68,84-hexaoxo-4,7,10,13,20,23,26,29,36,39,42,45,56,59,62,65,72,75,78,81,88,91,94, 97-tetracosaoxa-16,32,48,53,69,85-hexaazahectane-1,100-dioate (184)

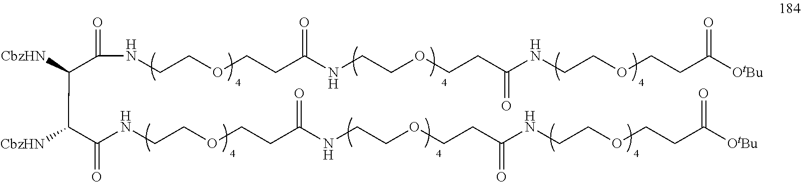

To a solution of compound 171 (0.26 g, 0.64 mmol) in DMA (10 mL) was added a solution of compound 183 (1.04 g, 1.28 mmol) in dichloromethane (5 mL), followed by DMAP (0.23 g, 1.92 mmol) and EDC HCl (0.36 g, 1.92 mmol). The mixture was stirred overnight, concentrated and purified on SiO₂ column, eluted with MeOH/CH₂Cl₂ (1:10) to afford compound 184 (0.81 g, 63% yield). ESI-MS m/z: [M+2H]²⁺ calcd for C₉₄H₁₆₂N₈O₃₈ 1006.55. found 1006.70.

### Example 78. Synthesis of (50R,51R)-di-tert-butyl 50,51-diamino-17,33,49,52,68,84-hexaoxo-4,7,10,13,20,23,26,29,36,39,42,45,56,59,62,65,72,75,78,81,88,91,94,97-tetracosaoxa-16,32,48,53,69,85-hexaazahectane-1,100-dioate (185)

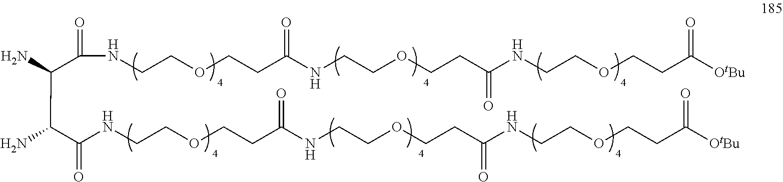

To a solution of compound 184 (0.81 g, 0.40 mmol) in MeOH (5 mL) was added 10% Pd/C (100 mg, 5 wt %), and the mixture was stirred under hydrogen atmosphere at r. t. for 18 h. Then the Pd/C catalyst was removed by filtration over Celite and the filter cake was washed with MeOH. The filtrate and washings were combined and concentrated to afford compound 185 (0.70 g, 100% yield). ESI-MS m/z: [M+2H]²⁺ calcd for C₇₈H₁₅₀N₈O₃₄: 872.52. found 872.55.

### Example 79. Synthesis of (50R,51R)-di-tert-butyl 50,51-bis(4-(2,5-dioxo-2,5-dihydro-1H-pyrrol-1-yl)butanamido)-17,33,49,52,68,84-hexaoxo-4,7,10,13,20,23,26,29,36,39,42,45,56, 59,62,65,72,75,78,81,88,91,94,97-tetracosaoxa-16,32,48,53,69,85-hexaazahectane-1,100-dioate (186), and (50S,51S)-50,51-bis(4-(2,5-dioxo-2,5-dihydro-1H-pyrrol-1-yl)butanamido)-17,33,49,52,68,84-hexaoxo-4,7,10,13,20,23,26,29,36,39,42,45,56,59,62,65,72,75,78,81,88,91, 94,97-tetracosaoxa-16,32,48,53,69,85-hexaazahectane-1,100-dioic acid (187)

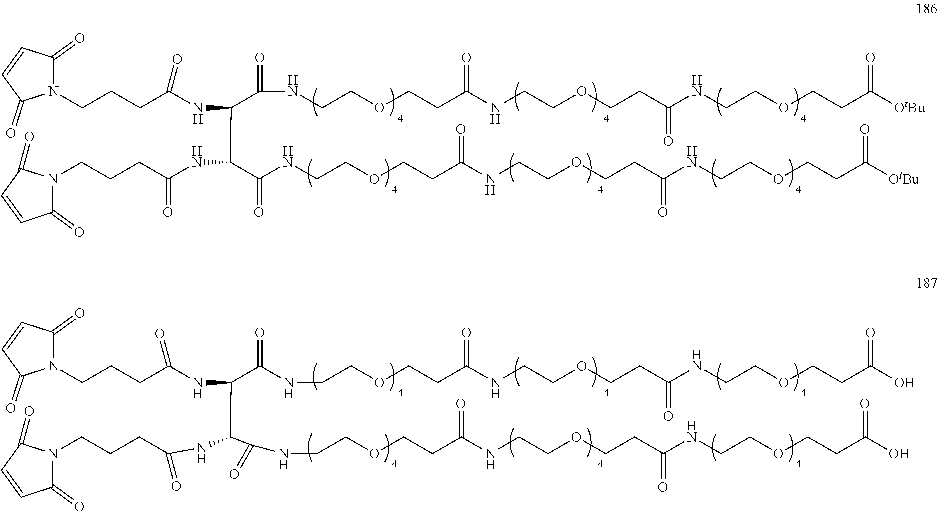

To a solution of 4-(2,5-dioxo-2,5-dihydro-1H-pyrrol-1-yl)butanoic acid (0.17 g, 1.00 mmol) and compound 185 (0.70 g, 0.40 mmol) in DMF (5 mL) were added N, N-diisopropylethylamine (0.88 mL, 5 mmol) and HATU (1.90 g, 12.56 mmol). The mixture was stirred overnight, concentrated and purified on SiO₂ column, eluted with 1-10% MeOH/CH₂Cl₂ to afford compound 186 as an oil, (0.548 g, 66% yield). ESI-MS m/z [M+2H]²⁺: calcd for C₉₄H₁₆₆N₁₀O₄₀ 2075.1264; found 2075.1350.

Compound 186 (0.54 g, 0.26 mmol) was dissolved in dichloromethane (5 mL) and treated with TFA (2.5 mL). The mixture was stirred at r.t. for 30 min, diluted with toluene (20 mL), concentrated to afford the title compound 187 (0.488, 96% yield) which was used for next step without further purification. ESI-MS m/z [M+H]⁺: calcd for C₈₆H₁₄₉N₁₀O₄₀ 1961.9933. found 1961.9987.

### Example 80. Synthesis of (S)-N,N′-(((((2S,53S,54S,105S)-53,54-bis(4-(2,5-dioxo-2,5-dihydro-1H-pyrrol-1-yl)butanamido)-2,105-dimethyl-4,20,36,52,55,71,87,103-octaoxo-7,10,13,16,23,26,29,32,39,42,45,48,59,62,65,68,75,78,81,84,91,94,97,100-tetracosaoxa-3,19,35,51,56,72,88,104-octaazahexahectane-1,106-dioyl)bis(azanediyl))bis(4,1-phenylene))bis(methylene))bis(1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium) (188)

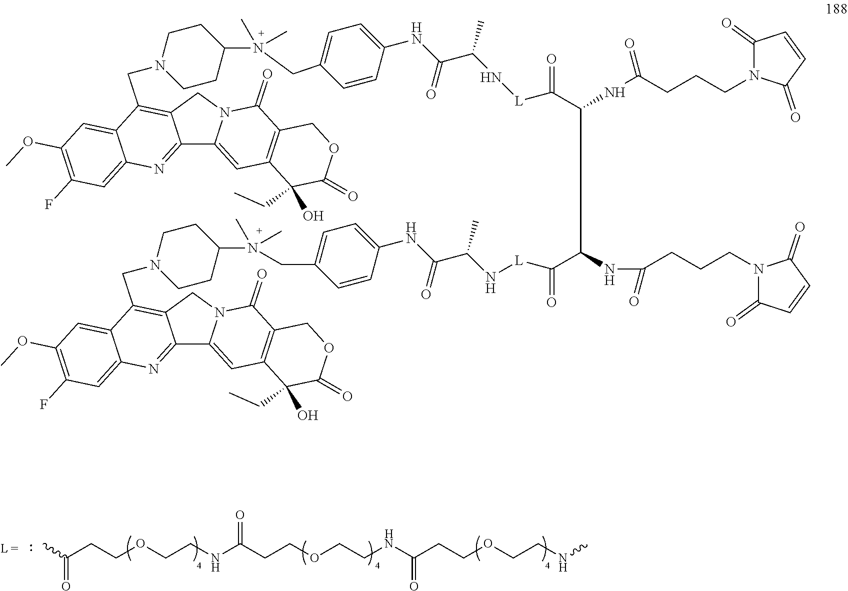

Compound 28 (47 mg, 0.060 mmol) and compound 187 (59 mg, 0.030 mmol) were dissolved in DMA (5 mL), cooled to about 0° C., and then N, N-diisopropylethylamine (21 μL, 0.12 mmol) was added. The reaction was warmed to r.t. and stirred for 2 hours, concentrated, and purified by preparative HPLC (acetonitrile/water containing formic acid) to give compound 188 (36 mg, 36% yield). ESI-MS m/z: M²⁺ calcd for C₁₆₄H₂₃₈F₂N₂₂O₅₀: 1675.8279. found 1675.8392.

### Example 81. Synthesis of tert-butyl 2,5,8,11,14,17,20,23,26-nonaoxaoctacosan-28-oate

(191)

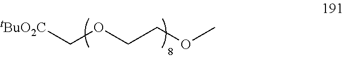

NaH (60%, 24 g, 600 mmol) was added to a solution of octaethylene glycol monomethyl ether (115 g, 300 mmol) in THF (3.0 L). After stirring at r.t. for 1 h, tert-butyl 2-bromoacetate (146 g, 750 mmol) was added to the mixture, and stirred at r.t. for 1 h. The mixture was then diluted with dichloromethane (4 L) and poured onto ice water (2 kg). The organic phase was separated and aqueous phase was extracted with dichloromethane (1 L). The combined organic phases were washed with water, dried over anhydrous Na₂SO₄. Purification by column chromatography (20% EtOAc/PE, then pure dichloromethane to 5% MeOH/dichloromethane) yielded the title compound as a yellow oil (108 g, 72% yield).

### Example 82. Synthesis of 2,5,8,11,14,17,20,23,26-nonaoxaoctacosan-28-oic acid (192)

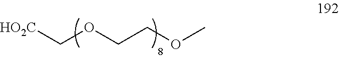

Tert-butyl 2,5,8,11,14,17,20,23,26-nonaoxaoctacosan-28-oate (210 g, 422 mmol) was dissolved in dichloromethane (400 mL) and anhydrous formic acid (1 L). The resulting solution was stirred at r.t. overnight. All volatiles were removed under vacuum, which afforded the title compound as a yellow oil (200 g, >100% yield).

### Example 83. Synthesis of 2,5,8,11,14,17,20,23,26-nonaoxaoctacosan-28-oyl chloride (193)

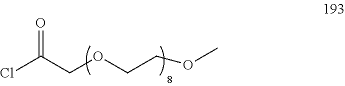

To the solution of 2,5,8,11,14,17,20,23,26-nonaoxaoctacosan-28-oic acid (198 g, 422 mmol) dissolved in dichloromethane (2.6 L), (COCl)₂ (275 mL) and DMF (0.5 mL) were added at r.t. The resulting solution was stirred at r.t. for 3 h. All volatiles were removed under vacuum to yield the title compound as a yellow oil (210 g, >100% yield).

### Example 84. Synthesis of (S)-34-(((benzyloxy)carbonyl)amino)-28-oxo-2,5,8,11,14,17,20,23,26-nonaoxa-29-azapentatriacontan-35-oic acid (194)

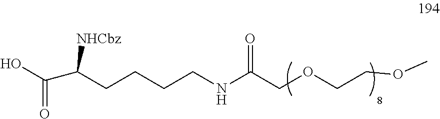

Z-L-Lys-OH (236 g, 844 mmol), Na₂CO₃ (89.5 g, 844 mmol) and NaOH (33.8 g, 844 mmol) were dissolved in water (1.6 L). The mixture was cooled under 0° C. using ice salt bath, to which a solution of 2,5,8,11,14,17,20,23,26-nonaoxaoctacosan-28-oyl chloride (210 g, 422 mmol) in THF (160 mL) was added. The resulting mixture was stirred at r.t. for 1 h, and then diluted with EtOAc (1 L). The aqueous layer was separated, to which concentrated HCl was added under ice cooling until pH 3 was reached. After extraction with dichloromethane, the organic layer was washed with brine, dried over Na₂SO₄ and concentrated to give the title compound as a yellow oil (290 g, 97% yield).

### Example 85. Synthesis of (S)-perfluorophenyl 34-(((benzyloxy)carbonyl)amino)-28-oxo-2,5,8,11,14,17,20,23,26-nonaoxa-29-azapentatriacontan-35-oate (195)

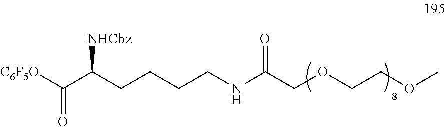

To a solution of compound 194 (183 g, 260 mmol) in dichloromethane (2 L) was added pentafluorophenol (95.4 g, 520 mmol) and DIC (131 g, 1.04 mol). The reaction was stirred at r.t. for 1 h, and then concentrated to give crude the title product (430 g).

### Example 86. Synthesis of (S)-tert-butyl 34-(((benzyloxy)carbonyl)amino)-28,35-dioxo-2,5,8,11,14,17,20,23,26-nonaoxa-29,36-diazatetracontan-40-oate (196)

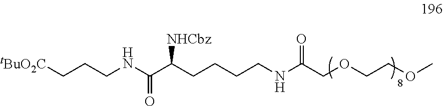

To a solution of tert-butyl 4-aminobutanoate (62.0 g, 390 mmol) in DMF (1.5 L) was added N, N-diisopropylethylamine (134 g, 1.04 mol) at 0° C. Compound 195 (430 g, crude) was then added at 10-20° C. and the resulting mixture was stirred at r.t. for 1 h. DMF was removed under vacuum and the residue was diluted with dichloromethane, washed with water. The aqueous phase was back-extracted with dichloromethane. The combined organic phase was washed with 0.2 N HCl and brine, dried over anhydrous Na₂SO₄, filtered and concentrated. Column chromatography (25% EtOAc/PE to pure EtOAc, then 0 to 5% MeOH/dichloromethane) gave the title compound as a yellow oil (180 g, 82% yield).

### Example 87. Synthesis of (S)-tert-butyl 34-amino-28,35-dioxo-2,5,8,11,14,17,20,23,26-nonaoxa-29,36-diazatetracontan-40-oate (197)

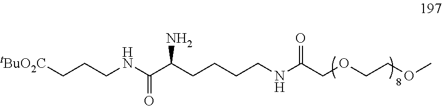

To a solution of compound 196 (78.0 g, 92.3 mmol, 1.0 eq.) in MeOH (500 mL) was added Pd/C (13 g, 10% Pd/C, 50% wet). The mixture was hydrogenated under 1 atm H₂ at r.t. overnight, then filtered and concentrated. The residue was purified by column chromatography (0 to 20% MeOH/dichloromethane) to give the title compound as a greenish yellow oil (70.2 g, 92% yield).

### Example 88. Synthesis of (7S,10R,11R,14S)-di-tert-butyl 10,11-bis(((benzyloxy)carbonyl)amino)-6,9,12,15-tetraoxo-7,14-bis(28-oxo-2,5,8,11,14,17,20,23,26-nonaoxa-29-azatritriacontan-33-yl)-5,8,13,16-tetraazaicosane-1,20-dioate (198)

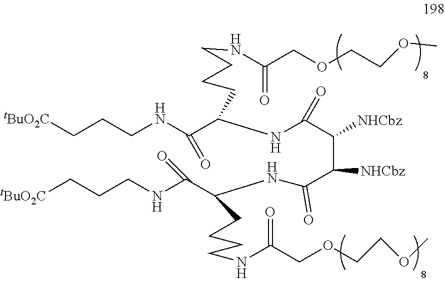

To a solution of compound 171 (0.85 g, 2.00 mmol) in DMA (10 mL) were added a solution of compound 197 (3.20 g, 4.50 mmol) in dichloromethane (10 mL), DMAP (1.50 g, 12 mmol) and EDC HCl (2.3 g, 12 mmol). The mixture was stirred overnight, concentrated and purified on SiO₂ column, eluted with EtOAc/CH₂Cl₂ (1:10) to afford compound 198 (3.33 g, 88% yield). ESI-MS m/z: [M+2H]² calcd for C₈₆H₁₄₆N₈O₃₂ 902.50. found 902.55.

### Example 89. Synthesis of (7S,10R,11R,14S)-di-tert-butyl 10,11-bis(4-(2,5-dioxo-2,5-dihydro-1H-pyrrol-1-yl)butanamido)-6,9,12,15-tetraoxo-7,14-bis(28-oxo-2,5,8,11,14,17,20,23,26-nonaoxa-29-azatritriacontan-33-yl)-5,8,13,16-tetraazaicosane-1,20-dioate (199)

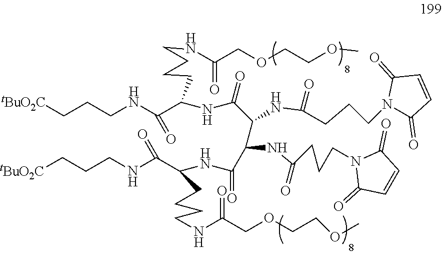

A mixture of compound 198 (3.33 g, 1.76 mmol) and Pd/C (5 wt %, 0.10 g) in dichloromethane (50 mL) was hydrogenated under 1 atm H₂ pressure overnight and then filtered over Celite (filter aid). The filtrate was concentrated and then dissolved in DMF (10 mL), to which EDC HCl (1.00 g, 5.28 mmol) and compound 4 (1.84 g, 5.28 mmol) were added. The mixture was stirred at r. t. for 16 h, concentrated and purified by SiO₂ column chromatography (1:4 MeOH/dichloromethane) to give an oil (2.56 g, 78% yield). ESI-MS m/z: [M+2H]²⁺ calcd for C₈₆H₁₄₈N₁₀O₃₄ 933.51. found 933.55.

### Example 90. Synthesis of (S)-N,N′-(((((2S,10S,13R,14R,17S,25S)-13,14-bis(4-(2,5-dioxo-2,5-dihydro-1H-pyrrol-1-yl)butanamido)-2,25-dimethyl-4,9,12,15,18,23-hexaoxo-10,17-bis(28-oxo-2,5,8,11,14,17,20,23,26-nonaoxa-29-azatritriacontan-33-yl)-3,8,11,16,19,24-hexaazahexa-cosane-1,26-dioyl)bis(azanediyl))bis(4,1-phenylene))bis(methylene))bis(1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium) formic acid salt (200)

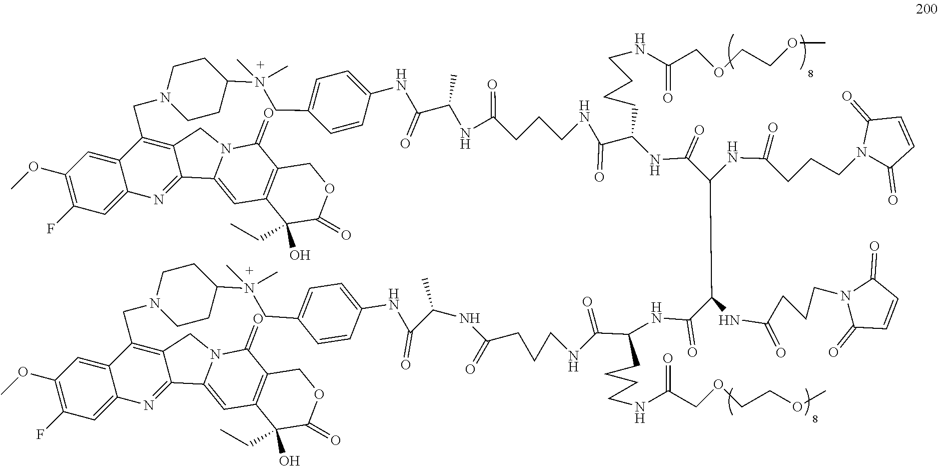

A mixture of compound 199 (1.00 g, 0.536 mmol) in dichloromethane (5 mL) and formic acid (5 mL) was stirred at r.t. for 24 h, and then concentrated. The residue was dissolved in DMA (5 mL), to which compound 28 (0.64 g, 0.89 mmol), triethylamine (0.15 mL, 1.07 mmol) and HATU (0.41 g, 1.07 mmol) were added and stirred at r.t. for 16 h. After the solvent was removed under high vacuum, the residue was purified by preparative HPLC (acetonitrile/water containing formic acid) (acetonitrile/water) to afford the compound 200 (1.06 g, 63% yield). ESI-MS m/z: M²⁺ calcd for C₁₅₆H₂₂₀F₂N₂₂O₄₄ 1571.78. found 1571.78.

### Example 91. Synthesis of methyl 4-(bis(2-hydroxyethyl)amino)-4-oxobutanoate (202)

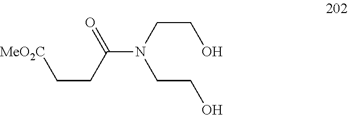 [SMILES: COC(=O)CCC(=O)N(CCO)CCO]

Dimethyl succinate (20.0 g, 136.9 mmol) and dihydroxyethylamine (7.20 g, 68.7 mmol) in a mixture of anhydrous toluene (500 mL) and pyridine (50 mL) were heated at 150° C. for 28 h. The mixture was concentrated and purified on silica gel column eluted with 5-25% ethyl acetate/dichloromethane to afford the title compound (12.5 g, 83% yield). ESI-MS m/z 242.42 ([M+Na]⁺).

### Example 92. Synthesis of methyl 4-(bis(2-((methylsulfonyl)oxy)ethyl) amino)-4-oxobutanoate (203)

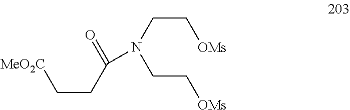 [SMILES: COC(=O)CCC(=O)N(CCOS(C)(=O)=O)CCOS(C)(=O)=O]

To a solution of methyl 4-(bis(2-hydroxyethyl)amino)-4-oxobutanoate (12.0 g, 49.56 mmol) in anhydrous pyridine (350 mL), methanesulfonyl chloride (20.0 g, 175.4 mmol) was added. After stirring overnight, the mixture was concentrated, diluted with ethyl acetate (350 mL), washed with cold 1 M NaH₂PO₄ (2×300 mL), dried over Na₂SO₄, filtered and evaporated to afford crude product (˜18.8 g, >100% yield). The crude product was used in the next step without further purification. ESI-MS m/z 376.06 ([M+H]⁺).

### Example 93. Synthesis of 3,6-endoxo-Δ-tetrahydrophthalimide (204)

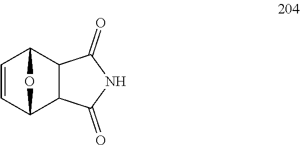 [SMILES: O=C1NC(=O)C2C1[C@@H]1C=C[C@H]2O1]

To a solution of maleimide (10.0 g, 103.0 mmol) in toluene (200 mL) was added furan (10.0 mL, 137.4 mmol). The mixture was heated in a 1 L auto Clave bomb at 100° C. for 8 h. The bomb was cooled to r. t., and the solid was rinsed out with MeOH, concentrated and crystallized in ethyl acetate/hexane to afford 16.7 g (99%) of the title compound. 1H NMR (CDCl₃): 11.12 (s, 1H), 6.68˜6.64 (m, 2H), 5.18˜5.13 (m, 2H), 2.97˜2.92 (m, 2H); ESI-MS m/z 188.04 ([M+Na]⁺).

### Example 94. Synthesis of Methyl 4-((2-((3aR,4R,7S,7aS)-1,3-dioxo-3a,4,7,7a-tetrahydro-1H-4,7-epoxyisoindol-2(3H)-yl)ethyl)(2-((4R,7S,7aS)-1,3-dioxo-3a,4,7,7a-tetrahydro-TH-4,7-epoxyisoindol-2(3H)-yl)ethyl)amino)-4-oxobutanoate (205)

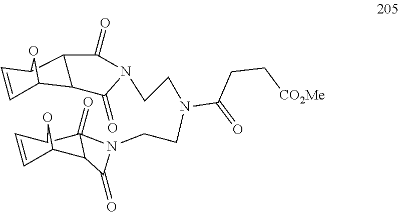

To a solution of methyl 4-(bis(2-((methylsulfonyl)oxy)ethyl)amino)-4-oxobutanoate (203, fresh made, 90% pure, 8.5 g, ˜20 mmol) in DMA (350 mL), 3,6-endoxo-Δ-tetrahydrophthalimide (204, 10.2 g, 61.8 mmol), sodium carbonate (8.0 g, 75.5 mmol) and sodium iodide (0.3 g, 2.0 mmol) were added. The mixture was stirred at r. t. overnight, concentrated, diluted with ethyl acetate (350 mL), washed with sat'ed NaHCO₃ solution (300 mL), brine (300 mL) and 1 M NaH₂PO₄ (300 mL). The organic layer was dried over sodium sulfate, filtered, evaporated, loaded on silica gel column and eluted with 10-30% ethyl acetate/hexane to afford the title compound (7.9 g, 77% yield). ESI-MS m/z 536.4 ([M+Na]⁺).

### Example 95. Synthesis of 4-(bis(2-(2,5-dioxo-2,5-dihydro-1H-pyrrol-1-yl)ethyl) amino)-4-oxobutanoic acid (206)

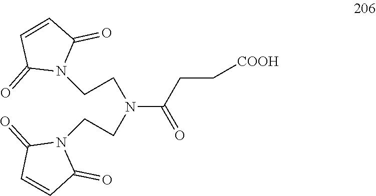 [SMILES: O=C(O)CCC(=O)N(CCN1C(=O)C=CC1=O)CCN1C(=O)C=CC1=O]

Compound 205 (3.0 g, 5.8 mmol) and trimethylstannanol (4.8 g, 26.4 mmol) in 1,2-dichloroethane (150 mL) were refluxed at 80° C. for 8 h, then cooled to r. t. and the residue was passed through a short silica gel column and eluted with dichloromethane/MeOH to remove excess trimethyltin hydroxide. Then the pooled fractions were combined, concentrated and diluted with DMA and toluene, heated to 120° C. and stirred overnight. The reaction mixture was loaded on silica gel column and eluted with 5-10% MeOH/dichloromethane to afford the title compound (1.62 g, 76% yield). ESI-MS m/z 386.2 ([M+Na]⁺).

### Example 96. Synthesis of 2,5-dioxopyrrolidin-1-yl 4-(bis(2-(2,5-dioxo-2,5-dihydro-1H-pyrrol-1-yl)ethyl)amino)-4-oxobutanoate (207)

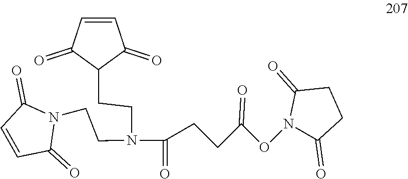 [SMILES: O=C(CCC(=O)N(CCC1C(=O)C=CC1=O)CCN1C(=O)C=CC1=O)ON1C(=O)CCC1=O]

To a solution of compound 206 (1.62 g, 4.46 mmol, 1.0 eq.) in 10 mL of DMA, N-hydroxysuccinimide (0.61 g, 5.35 mmol, 1.2 eq.) and EDC·HCl (1.71 g, 8.92 mmol, 2.0 eq.) were added. The reaction was stirred at r.t. overnight and then washed with water (50 mL×2), brine (100 mL), dried over anhydrous sodium sulfate, and concentrated to give an oil (2.00 g).

The crude product was used directly in the next step. ESI-MS m/z [M+H]⁺: calcd for C₂₀H₂₀N₄O₉ 461.12, found: 461.24.

### Example 97. Synthesis of N-(4-((S)-2-(4-(bis(2-(2,5-dioxo-2,5-dihydro-1H-pyrrol-1-yl)ethyl)amino)-4-oxobutanamido)propanamido)benzyl)-1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium (208)

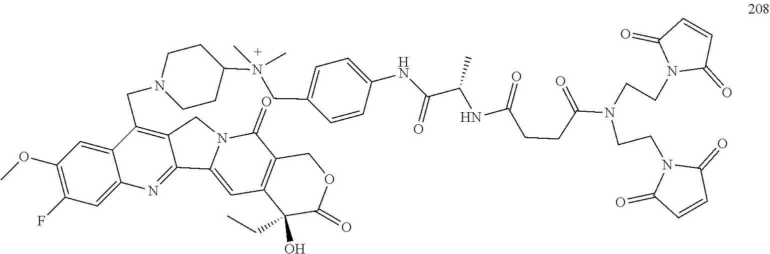

The crude product from the previous step (0.20 g) was dissolved in DMA (5 mL), to which compound 28 (0.71 g, 1.00 mmol) and N, N-diisopropylethylamine (0.20 mL, 1.20 mmol) were added at 0° C. The reaction was warmed to r.t. and stirred for 2 hours, concentrated, and purified by preparative HPLC (acetonitrile/water containing formic acid) to give compound 208 (0.85 g, 80% yield). ESI-MS m/z: M+ calcd for C₅₅H₆₁FN₉O₁₂: 1058.44. found 1058.60.

### Example 98. Synthesis of (S)-tert-butyl 34-(4-(bis(2-(2,5-dioxo-2,5-dihydro-1H-pyrrol-1-yl)ethyl)amino)-4-oxobutanamido)-28,35-dioxo-2,5,8,11,14,17,20,23,26-nonaoxa-29,36-diazatetracontan-40-oate (210)

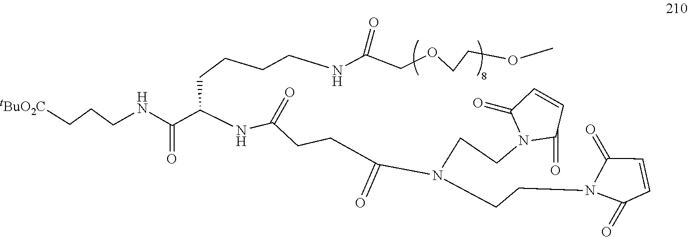

To a solution of compound 197 (2.98 g, 4.20 mmol) and compound 206 (1.39 g, 3.82 mmol) in DMA (20 mL), EDC HCl (0.80 g, 4.20 mmol) was added. The reaction was stirred at r.t. overnight, then poured onto water (50 mL) and extracted with ethyl acetate (3×40 mL). The combined organic phase was washed with brine (40 mL), dried over anhydrous sodium sulfate, filtered and concentrated. The residue was purified by column chromatography (10-50% ethyl acetate/petroleum ether) to give a colorless oil (3.23 g, 80% yield). ESI-MS m/z 1057.85 ([M+H]⁺).

### Example 99. Synthesis of (S)-34-(4-(bis(2-(2,5-dioxo-2,5-dihydro-1H-pyrrol-1-yl)ethyl)amino)-4-oxobutanamido)-28,35-dioxo-2,5,8,11,14,17,20,23,26-nonaoxa-29,36-diazatetracontan-40-oic acid (211)

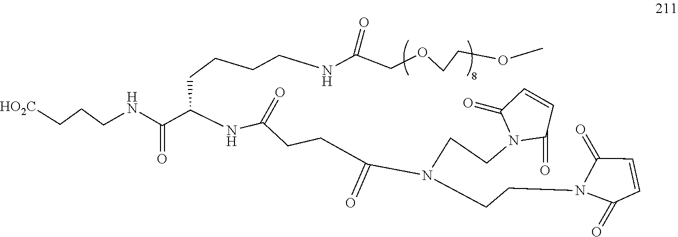

A solution of compound 210 (3.20 g, 3.03 mmol) in formic acid (10 mL) and dichloromethane (5 mL) was stirred at r.t. overnight. The solution was then concentrated and co-evaporated with toluene three times to give a colorless oil (3.00 g, crude), which was used without further purification. ESI-MS m/z 1001.50 ([M+H]⁺).

### Example 100. Synthesis of (S)-2,5-dioxopyrrolidin-1-yl 34-(4-(bis(2-(2,5-dioxo-2,5-dihydro-1H-pyrrol-1-yl)ethyl)amino)-4-oxobutanamido)-28,35-dioxo-2,5,8,11,14,17,20,23,26-nonaoxa-29,36-diazatetracontan-40-oate (212)

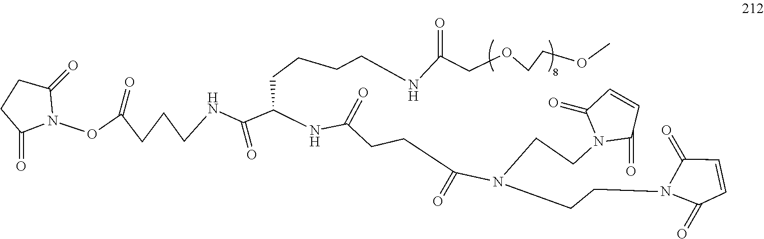

To a solution of compound 211 (3.00 g, crude, 3.03 mmol) in DMA (15.0 mL), N-hydroxysuccinimide (0.38 g, 3.33 mmol) and EDC HCl (0.87 g, 4.55 mmol) were added, and the reaction was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified by silica gel column (10-50% ethyl acetate/petroleum ether) to give a colorless oil (2.90 g, 90% yield). ESI-MS m/z 1098.50 ([M+H]⁺).

### Example 101. Synthesis of N-(4-((34S,42S)-34-(4-(bis(2-(2,5-dioxo-2,5-dihydro-1H-pyrrol-1-yl)ethyl)amino)-4-oxobutanamido)-42-methyl-28,35,40-trioxo-2,5,8,11,14,17,20,23,26-nonaoxa-29,36,41-triazatritetracontanamido)benzyl)-1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium (213)

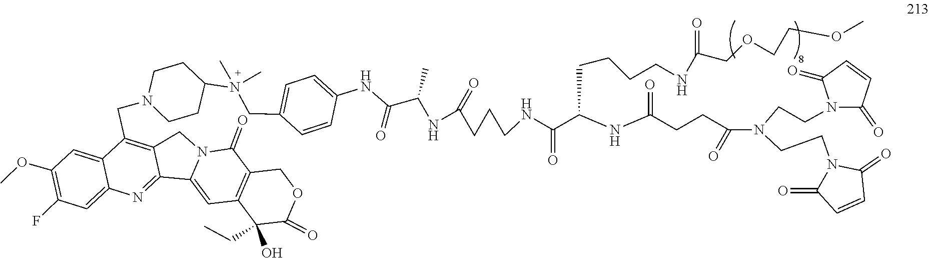

Compound 212 (0.10 g, 0.091 mmol) was dissolved in DMA (5 mL), to which compound 28 (56.8 mg, 0.08 mmol) and N, N-diisopropylethylamine (0.020 mL, 0.12 mmol) were added at 0° C. The reaction was warmed to r.t. and stirred for 2 hours, concentrated, and purified by preparative HPLC (acetonitrile/water containing formic acid) to give compound 213 (84 mg, 62% yield). ESI-MS m/z: M+ calcd for C₈₄H₁₁₆FN₁₂O₂₄: 1695.82. found 1695.82.

### Example 102. Synthesis of tert-butyl 2-(2-(1,3-dioxoisoindolin-2-yl)acetyl)hydrazinecarboxylate (216)

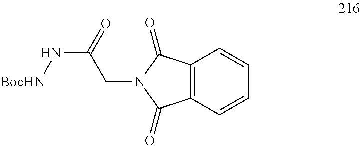 [SMILES: CC(C)(C)OC(=O)NNC(=O)CN1C(=O)c2ccccc2C1=O]

To a solution of Boc-hydrazine (7.08. g, 53.5 mmol) in dichloromethane (200 mL) at 0° C., triethylamine (13.5 mL, 97.4 mmol) and compound 215 (10.8 g, 48.7 mmol) was added in sequence. After stirred at r.t. for 30 min, the mixture was poured into ice-water (100 mL) and extracted with dichloromethane (3×100 mL). The combined organic phases were washed with water (100 mL) and brine (100 mL), dried over anhydrous sodium sulfate, filtered and concentrated to give a white solid (15.5 g, 100% yield). ESI-MS m/z 320.12 ([M+H]⁺).

### Example 103. Synthesis of 2-(1,3-dioxoisoindolin-2-yl)acetohydrazide (217)

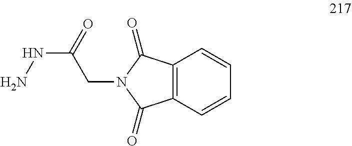 [SMILES: NNC(=O)CN1C(=O)c2ccccc2C1=O]

Compound 216 (15.5 g, 48.7 mmol) was dissolved in 1,4-dioxane (150 mL) and treated with 25% HCl (50 mL) at r.t. for 1 h. The reaction mixture was concentrated and then co-evaporated with toluene to give a white solid (10.6 g, 100% yield). ESI-MS m/z 220.06 ([M+H]⁺).

### Example 104. Synthesis of 2-(1,3-dioxoisoindolin-2-yl)-N′-(2-(1,3-dioxoisoindolin-2-yl)acetyl)acetohydrazide (218)

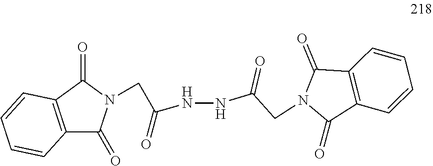 [SMILES: O=C(CN1C(=O)c2ccccc2C1=O)NNC(=O)CN1C(=O)c2ccccc2C1=O]

To a solution of compound 217 (10.6 g, 48.7 mmol) in THF (200 mL) at 0° C., triethylamine (13.5 mL, 97.4 mmol) and compound 215 (10.8 g, 48.7 mmol) were added. The reaction was warmed to r.t. and stirred overnight. The precipitate was collected by filtration and suspended in water (100 mL) and stirred for 20 min. The mixture was filtered again and a white solid (15.7 g, 80% yield) was collected as compound 218. ESI-MS m/z 407.09 ([M+H]⁺).

### Example 105. Synthesis of di-tert-butyl 2,2′-(1,2-bis(2-(1,3-dioxoisoindolin-2-yl)acetyl)hydrazine-1,2-diyl)diacetate (219)

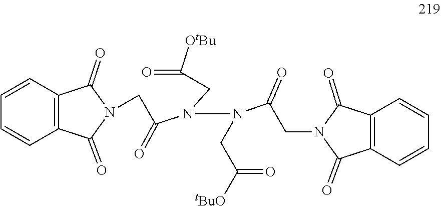 [SMILES: CC(C)(C)OC(=O)CN(C(=O)CN1C(=O)c2ccccc2C1=O)N(CC(=O)OC(C)(C)C)C(=O)CN1C(=O)c2ccccc2C1=O]

NaH (0.5 g, 12.3 mmol) was added to a solution of compound 218 (2.0 g, 4.92 mmol) in DMF (40 mL) at 0° C. in portions. The mixture was warmed to r.t. and stirred for 3 h. After that tert-butyl bromoacetate (2.0 g, 10.3 mmol) was added and the reaction was stirred overnight before pouring into ice-water (100 mL) and extracting with dichloromethane (3×50 mL). The combined organic phase was washed with water (50 mL), brine (50 mL), dried over anhydrous sodium sulfate, filtered and concentrated, purified by silica gel chromatography to give a white solid (1.5 g, 50% yield). ESI-MS m/z 635.23 ([M+H]⁺).

### Example 106. Synthesis of di-tert-butyl 2,2′-(1,2-bis(2-aminoacetyl)hydrazine-1,2-diyl)diacetate (220)

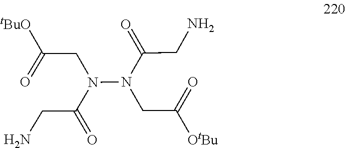 [SMILES: CC(C)(C)OC(=O)CN(C(=O)CN)N(CC(=O)OC(C)(C)C)C(=O)CN]

A mixture of compound 219 (1.5 g, 2.36 mmol) and hydrazine (442 mg, 7.08 mmol) in ethanol (30 mL) was refluxed for 1 h, then cooled to r.t. and filtered. The filtrate was concentrated and taken up in ethyl acetate (20 mL), filtered again. The filtrate was concentrated to give a white solid 220 (750 mg, 85% yield). ESI-MS m/z 375.22 ([M+H]⁺).

### Example 107. Synthesis of di-tert-butyl 2,2′-(1,2-bis(2-(2,5-dioxo-2,5-dihydro-1H-pyrrol-1-yl)acetyl)hydrazine-1,2-diyl)diacetate (221)

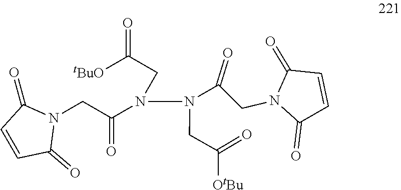 [SMILES: CC(C)(C)OC(=O)CN(C(=O)CN1C(=O)C=CC1=O)N(CC(=O)OC(C)(C)C)C(=O)CN1C(=O)C=CC1=O]

A solution of compound 220 (750 mg, 2 mmol) in THF (20 mL) and saturated NaHCO₃ aqueous solution (30 mL) at 0° C., N-methoxycarbonyl maleimide (622 mg, 4 mmol) was added. The reaction mixture was stirred at 0° C. for 1 h. A white solid was collected by filtration as compound 221 (854 mg, 80% yield). ESI-MS m/z 535.20 ([M+H]⁺).

### Example 108. Synthesis of 2,2′-(1,2-bis(2-(2,5-dioxo-2,5-dihydro-1H-pyrrol-1-yl)acetyl)hydrazine-1,2-diyl)diacetic acid (222)

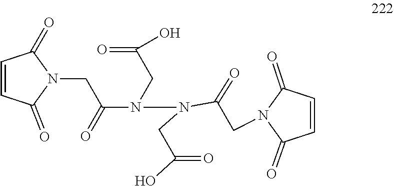 [SMILES: O=C(O)CN(C(=O)CN1C(=O)C=CC1=O)N(CC(=O)O)C(=O)CN1C(=O)C=CC1=O]

Compound 221 (854 mg, 1.6 mmol) was dissolved in dioxane (3 mL) and treated with 25% HCl (3 mL) at r.t. for 2 h. The reaction was then evaporated to give compound 222 (675 mg, 100% yield). ESI-MS m/z 423.07 ([M+H]⁺).

### Example 109. Synthesis of di-tert-butyl 4,4′-((2,2′-(1,2-bis(2-(2,5-dioxo-2,5-dihydro-1H-pyrrol-1-yl)acetyl)hydrazine-1,2-diyl)bis(acetyl))bis(azanediyl))dibutanoate (223)

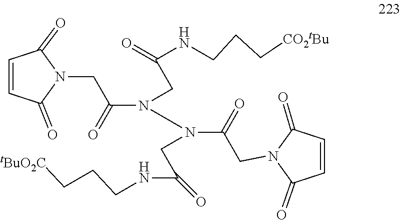 [SMILES: CC(C)(C)OC(=O)CCCNC(=O)CN(C(=O)CN1C(=O)C=CC1=O)N(CC(=O)NCCCC(=O)OC(C)(C)C)C(=O)CN1C(=O)C=CC1=O]

To a solution of compound 222 (200 mg, 0.47 mmol) in DMF (5 mL) at 0° C., tert-butyl 4-aminobutanoate (158 mg, 0.99 mmol) and EDC HCl (189.7 mg, 0.99 mmol) were added. The reaction mixture was warmed to r.t. and stirred overnight, poured into ice-water, and extraction with dichloromethane (3×10 mL). The combined organic phase was washed with 0.2 N HCl (5 mL), water (5 mL), brine (5 mL), dried over anhydrous sodium sulfate, filtered and concentrated to give a white solid (330 mg, 100% yield).

### Example 110. Synthesis of bis(2,5-dioxopyrrolidin-1-yl) 4,4′-((2,2′-(1,2-bis(2-(2,5-dioxo-2,5-dihydro-1H-pyrrol-1-yl)acetyl)hydrazine-1,2-diyl bis acetyl))bis(azanediyl))dibutanoate (225)

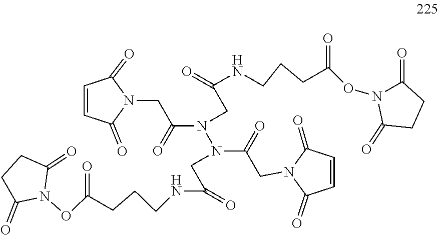 [SMILES: O=C(CN(C(=O)CN1C(=O)C=CC1=O)N(CC(=O)NCCCC(=O)ON1C(=O)CCC1=O)C(=O)CN1C(=O)C=CC1=O)NCCCC(=O)ON1C(=O)CCC1=O]

Compound 223 (330 mg, 0.47 mmol) was dissolved in dioxane (3 mL) and treated with 25% HCl (3 mL) at r.t. for 2 h. The reaction was concentrated and re-dissolved in DMF (5 mL) and cooled to 0° C., N-hydroxysuccinimide (113 mg, 0.98 mmol) and EDC HCl (189 mg, 0.98 mmol) were added in sequence. The reaction was warmed to r.t. and stirred overnight, poured into ice-water, and extraction with dichloromethane (3×20 mL). The combined organic phase was washed with water (5 mL), brine (5 mL), dried over anhydrous sodium sulfate, filtered and concentrated to give a white solid 225 (369 mg, 100% yield). ESI-MS m/z 787.21 ([M+H]⁺).

### Example 111. Synthesis of (S)-N,N′-(((((2S,21S)-11,12-bis(2-(2,5-dioxo-2,5-dihydro-1H-pyrrol-1-yl)acetyl)-2,21-dimethyl-4,9,14,19-tetraoxo-3,8,11,12,15,20-hexaazadocosane-1,22-dioyl)bis(azanediyl))bis(4,1-phenylene))bis(methylene))bis(1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium) 226

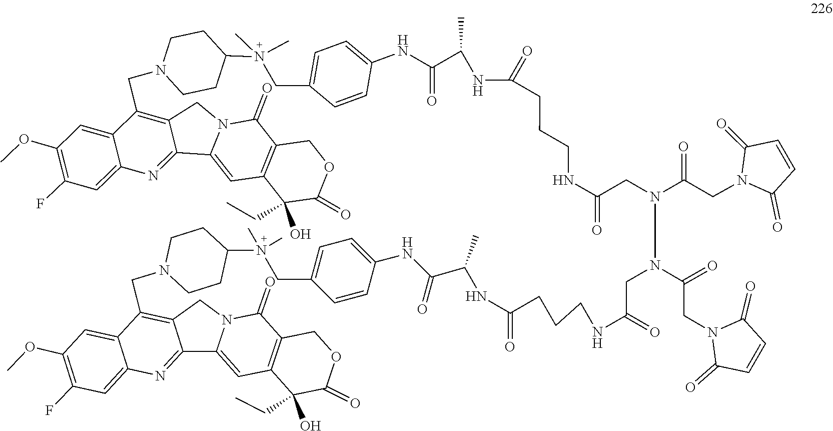

Compound 225 (31.5 mg, 0.04 mmol) was dissolved in DMA (5 mL), to which compound 28 (56.8 mg, 0.08 mmol) and N, N-diisopropylethylamine (0.020 mL, 0.12 mmol) were added at 0° C. The reaction was warmed to r.t. and stirred for 2 hours, concentrated, and purified by preparative HPLC (acetonitrile/water containing formic acid) to give compound 226 (57 mg, 72% yield). ESI-MS m/z: M²⁺ calcd for C₁₀₂H₁₁₆F₂N₁₈O₂: 991.42. found 991.86.

### Example 112. Synthesis of tert-butyl 3-((2-aminoethyl)amino)propanoate (228)

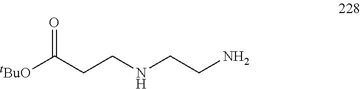 [SMILES: CC(C)(C)OC(=O)CCNCCN]

Tert-butyl acrylate (12.81 g, 0.10 mmol) and ethane-1,2-diamine (24.3 g, 0.40 mol) in THF (150 mL) was stirred at 45° C. for 24 h. The mixture was concentrated and purified on Al₂O₃ gel column eluted with triethylamine/MeOH/CH₂Cl₂ (5%:15%:80%) to afford the title compound (17.50 g, 92% yield). ESI-MS m/z 189.20 ([M+H]⁺).

### Example 113. Synthesis of 3-((2-aminoethyl)amino)propanoic acid, HCl salt (229)

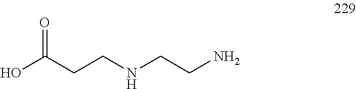

Tert-butyl 3-((2-aminoethyl)amino)propanoate (17.00 g, 90.33 mmol) in 1,4-dioxane (50 mL) was treated with conc. HCl (15 mL). The mixture was stirred at r. t. for 30 min, concentrated and diluted with pure water (150 mL) and EtOAc/PE (40 mL, 1:5). The mixture was separated, and the organic layer was extracted with water (2×10 mL). The aqueous layer was concentrated and dried on a vacuum pump to afford the title compound (18.70 g, 100% yield, and 96% pure by LC-MS). ESI-MS m/z 133.20 ([M+H]⁺).

### Example 114. Synthesis of 3-((2-(2,5-dioxo-2,5-dihydro-1H-pyrrol-1-yl)ethyl)amino)-propanoic acid (230)

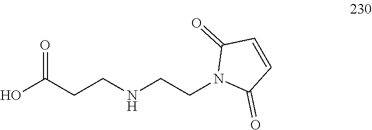 [SMILES: O=C(O)CCNCCN1C(=O)C=CC1=O]

To a solution of 3-((2-aminoethyl)amino)propanoic acid (18.70 g, 90.33 mmol) in THF (150 mL) at 0° C., maleic anhydride (8.85 g, 90.33 mmol) was added. The mixture was stirred at 0-4° C. for 4 h, concentrated to afford (Z)-4-((2-((2-carboxyethyl)amino)ethyl)amino)-4-oxobut-2-enoic acid in quantitative yield, confirmed by LC-MS. Then toluene (150 mL) and DMA (50 mL) were added to the mixture, refluxed at 90° C. with Dean-Stark trap. After collected 30 mL solvent in the trap, hexamethyldisilane (9.0 mL, 43.15 mmol) and ZnCl₂ (16 mL, 1.0 M in diethyl ether) were added. The mixture was heated to 115-125° C., and toluene was collected through a Dean-Stark trap. The reaction mixture was fluxed at 120° C. for 6 h. During this period, 2×40 mL of dry toluene was added to keep the mixture volume around 50 mL. Then the mixture was cooled and 1 mL of 1:10 HCl (conc)/CH₃OH was added in. The mixture was evaporated, and purified on SiO₂ column eluted with water/CH₃CN (1:15), and dried on a vacuum pump to afford the title compound 14.75 g (77.0% yield). ESI-MS m/z 213.10 ([M+H]⁺).

### Example 115. Synthesis of 2,5,8,11,14,17,20,23-octaoxapentacosan-25-yl 4-methylbenzenesulfonate (231)

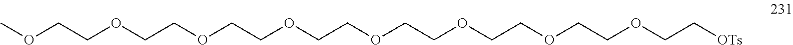 [SMILES: COCCOCCOCCOCCOCCOCCOCCOCCOS(=O)(=O)c1ccc(C)cc1]

To a solution of 2,5,8,11,14,17,20,23-Octaoxapentacosan-25-ol (50.0 g, 0.130 mol) in dichloromethane (200 mL) and pyridine (100 mL), TsCl (30.2 g, 0.159 mol) was added. The mixture was stirred overnight, evaporated and purified on SiO₂ column eluted with acetone/dichloromethane (1:1 to 4:1), and dried on a vacuum pump to afford the title compound 57.34 g (82.0% yield). ESI-MS m/z 539.40 ([M+H]⁺).

### Example 116. Synthesis of S-2,5,8,11,14,17,20,23-octaoxapentacosan-25-yl ethanethioate (232)

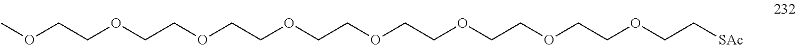

To a solution of 2,5,8,11,14,17,20,23-octaoxapentacosan-25-yl 4-methylbenzenesulfonate (57.30 g, 0.106 mol) in the mixture of THF (300 mL) and N, N-diisopropylethylamine (50 mL), HSAc (10.0 g, 0.131 mol) was added. The mixture was stirred overnight, evaporated and purified on SiO₂ column eluted with EtOAc/dichloromethane (1:2 to 4:1), and dried on a vacuum pump to afford the title compound 40.51 g (86% yield). ESI-MS m/z 443.35 ([M+H]⁺).

### Example 117. Synthesis of 2,5,8,11,14,17,20,23-octaoxapentacosane-25-sulfonic acid (233)

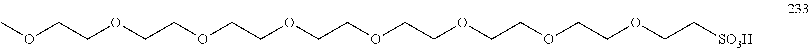 [SMILES: COCCOCCOCCOCCOCCOCCOCCOCCS(=O)(=O)O]

S-2,5,8,11,14,17,20,23-octaoxapentacosan-25-yl ethanethioate (40.40 g, 0.091 mol) in the mixture of acetic acid (200 mL) and 30% H₂O₂ (100 mL) was stirred at 35° C. overnight. The mixture was concentrated, diluted with pure water (200 mL) and toluene (150 mL), separated and the organic layer was extracted with water (2×25 mL). The aqueous solutions were combined, evaporated and dried on a vacuum pump to afford the title compound 40.50 g (99% yield, 95% pure by LC-MS). ESI-MS m/z 449.30 ([M+H]⁺).

### Example 118. Synthesis of 3,3-N,N-(2″-maleimidoethyl)(2′,5′,8′,11′,14′,17′,20′,23′,26′-nonaoxaoctacosane-28′-sulfin)aminopropanoic acid (234)

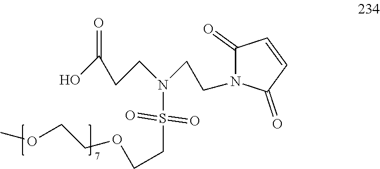

To a solution of 2,5,8,11,14,17,20,23-octaoxapentacosane-25-sulfonic acid (20.0 g, 44.62 mmol) in the mixture of THF (100 mL) and dichloromethane (100 mL), (COCl)₂ (25.21 g, 200.19 mmol) and DMF (0.015 mL) was added in sequence. The mixture was stirred at RT for 2 h, concentrated, co-evaporated with dichloromethane/toluene (1:1, 2×50 mL) and then re-dissolved in THF (50 mL). To the compound of 3-((2-(2,5-dioxo-2,5-dihydro-1H-pyrrol-1-yl)ethyl)amino)-propanoic acid (7.50 g, 35.36 mmol) in THF (100 mL) was added above sulfonyl chloride solution. The mixture was stirred overnight, evaporated in vacuo and purified on SiO₂ column eluted with MeOH/dichloromethane (1:6 to 1:5), and dried on a vacuum pump to afford the title compound 14.76 g (65% yield). ESI-MS m/z 643.35 ([M+H]⁺).

### Example 119. Synthesis of N— N-succinimido 3,3-N,N-(2″-maleimidoethyl) (2′,5′,8′,11′,14′,17′,20′,23′,26′-nonaoxaoctacosane-28′-sulfin)aminopropanoate (235)

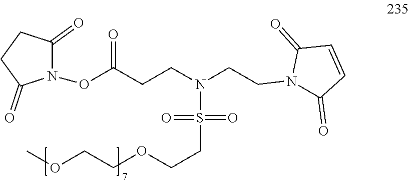

A mixture of 3,3-N,N-(2″-maleimidoethyl)(2′,5′,8′,11′,14′,17′,20′,23′,26′-nonaoxaoctacosane-28′-sulfin)aminopropanoic acid (234) (7.50 g, 11.67 mmol), N-hydroxysuccinimide (1.50 g, 13.04 mmol) and EDC·HCl (10.10 g, 52.60 mmol) in THF (100 mL) was stirred overnight, evaporated under vacuum and purified on SiO₂ column eluted with EtOAc/dichloromethane (1:4 to 2:1), and dried on a vacuum pump to afford the title compound 6.30 g (73% yield). ESI-MS m/z 740.40 ([M+H]⁺).

### Example 120. Synthesis of Compound 236

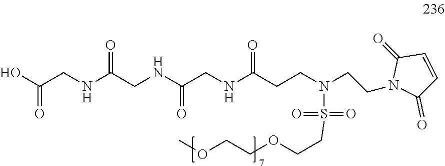

A solution of H-Gly-Gly-Gly-OH (0.50 g, 2.03 mmol) and compound 235 (1.65 g, 2.22 mmol) in DMF (15 mL) at 0° C., N, N-diisopropylethylamine (3 mL) was added. The reaction mixture was stirred at 0° C. for 0.5 h, at r. t. for 4 h. Then the reaction mixture was concentrated, and purified by SiO₂ chromatography (acetonitrile/water 95:5 with 0.1% formic acid) to afford the title compound (1.04 g, 63% yield). ESI-MS m/z [M+H]⁺: calcd for C₃₂H₅₆N₅O₁₇S 814.33; found, 814.46.

### Example 121. Synthesis of Compound 237

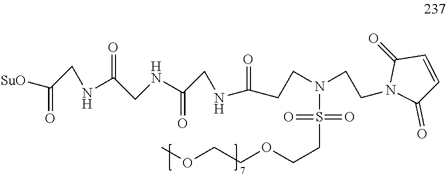

A mixture of compound 236 (0.70 g, 0.86 mmol), N-hydroxysuccinimide (0.20 g, 1.73 mmol) and EDC·HCl (1.21 g, 6.36 mmol) in THF (20 mL) was stirred overnight, evaporated in vacuo and purified on SiO₂ column, eluted with EtOAc/dichloromethane (1:4 to 2:1), and dried on a vacuum pump to afford the title compound (0.540 g, 69% yield). ESI-MS m/z [M+H]⁺: calcd for C₃₆H₅₉N₆O₁₉S, 911.34. found 911.42.

### Example 122. Synthesis of Compound 238

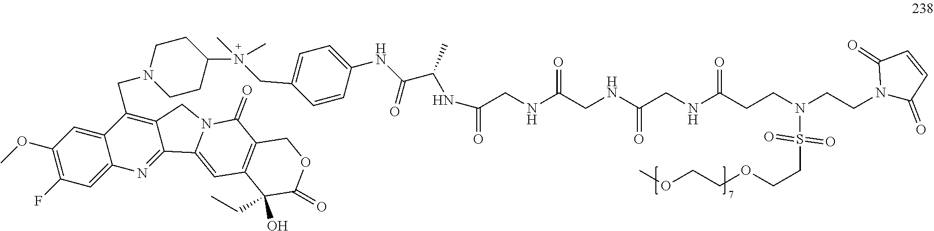

Compound 237 (36 mg, 0.04 mmol) was dissolved in DMF (5 mL), to which compound 28 (56.8 mg, 0.08 mmol) and N, N-diisopropylethylamine (0.020 mL, 0.12 mmol) were added at 0° C. The reaction was warmed to r.t. and stirred for 2 hours, concentrated, and purified by preparative HPLC (acetonitrile/water containing formic acid) to give compound 238 (48 mg, 80% yield). ESI-MS m/z: M⁺ calcd for C₇₁H₉₉FN₁₁O₂₂S 1508.67. found 1508.86.

### Example 123. Synthesis of Methyl 4-(bis(2-(acetylthio)ethyl)amino)-4-oxobutanoate (240)

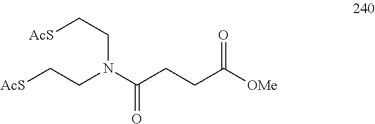

Methyl 4-(bis(2-((methylsulfonyl)oxy)ethyl)amino)-4-oxobutanoate (fresh made, 90% pure, 8.5 g, ˜20 mmol) in DMA (350 mL) at 0° C. was added thioacetic acid (10 mL, 134 mmol), followed by triethylamine (30 mL, 215 mmol). The mixture was then stirred at r. t. overnight, concentrated, diluted with EtOAc (350 mL), washed with sat'ed NaHCO₃ (300 mL), brine (300 mL) and 1 M NaH₂PO₄ (300 mL). The organic layer was dried over Na₂SO₄, filtered, evaporated and purified on SiO₂ column eluted with EtOAc/hexane (10%˜25% EtOAc) to afford the title compound (5.1 g, 76% yield). ESI-MS m/z [M+Na]⁺: calcd for C₁₃H₂₁NO₅S₂ 358.1. found 358.2.

### Example 124. Synthesis of 4-(Bis(2-(pyridin-2-yldisulfanyl)ethyl)amino)-4-oxobutanoic acid (241)

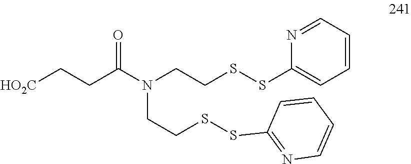 [SMILES: O=C(O)CCC(=O)N(CCSSc1ccccn1)CCSSc1ccccn1]

Methyl 4-(bis(2-(acetylthio)ethyl)amino)-4-oxobutanoate (5.0 g, 14.9 mmol) in THF (150 mL) was added NaOH (5.0 g, 125 mmol) in water (100 mL). The mixture was stirred at r.t. for 35 min, neutralized with H₃PO₄ to pH 7. Then PySSPy (26.0 g, 118 mmol) in THF (100 mL) was added and the mixture was stirred for 4 h, concentrated and purified on SiO₂ column, eluted with MeOH/dichloromethane/HOAc (1:20/0.2) to afford the title product (5.8 g, 85.6% yield). ESI-MS m/z [M+Na]⁺: calcd for C₁₈H₂₁N₃O₃S₄ 478.0. found 478.2.

### Example 125. Synthesis of 2,5-dioxopyrrolidin-1-yl 4-(bis(2-(pyridin-2-yldisulfanyl)ethyl)amino)-4-oxobutanoate (242)

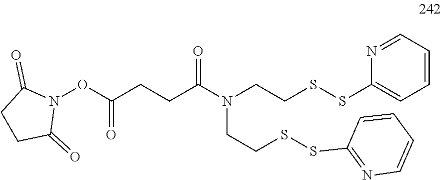 [SMILES: O=C(CCC(=O)N(CCSSc1ccccn1)CCSSc1ccccn1)ON1C(=O)CCC1=O]

To a solution of 4-(Bis(2-(pyridin-2-yldisulfanyl)ethyl)amino)-4-oxobutanoic acid (5.2 g, 11.5 mmol) in DMA (100 mL) were added N-hydroxysuccinimide (1.6 g, 13.9 mmol) and EDC HCl (5.0 g, 26.1 mmol). The mixture was stirred overnight, evaporated and purified on SiO₂ column, eluted with EtOAc/dichloromethane (5% to 15% EtOAc) to afford the title product (5.8 g, 85.6% yield). ESI-MS m/z [M+Na]⁺: calcd for C₂₂H₂₄N₄O₅S₄ 575.1. found 575.2.

### Example 126. Synthesis of N-(4-((S)-2-(4-(bis(2-(pyridin-2-yldisulfanyl)ethyl)amino)-4-oxobutanamido)propanamido)benzyl)-1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium (243)

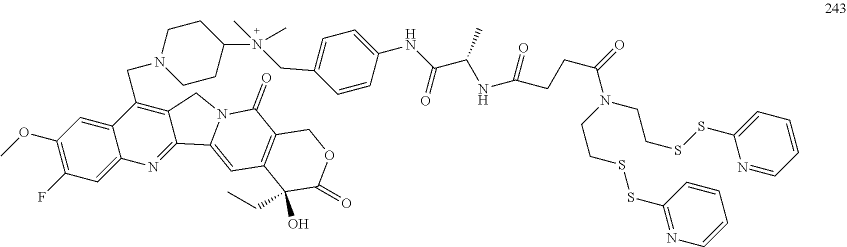

Compound 242 (23 mg, 0.04 mmol) was dissolved in DMA (5 mL), to which compound 28 (56.8 mg, 0.08 mmol) and N, N-diisopropylethylamine (0.020 mL, 0.12 mmol) were added at 0° C. The reaction was warmed to r.t. and stirred for 2 hours, concentrated, and purified by preparative HPLC (acetonitrile/water containing formic acid) to give compound 273 (39 mg, 85% yield). ESI-MS m/z: M⁺ calcd for C₅₇H₆₅FN₉O₈S₄: 1150.38. found 1150.45.

### Example 127. Synthesis of 4-(2-Pyridyldithio)-4-methylpentanoic acid (245)

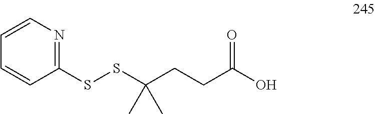 [SMILES: CC(C)(CCC(=O)O)SSc1ccccn1]

4-Mercapto-4-methylpentanoic Acid (Goff, D. et al, BioConjugate Chem. 1990, 1, 381-386) (4.67 g, 31.5 mmol) in MeOH (15 mL) was added the solution of 2,2′-dithiodipyridine (30.0 g, 136.2 mmol) in the mixture of MeOH (80 mL) and 100 mM sodium phosphate buffer solution (pH 7.5, 70 mL). After stirred for 6 h, the mixture was concentrated, extracted with EtOAc/Hexane (1:1). The aqueous solution was adjusted to pH 3 and extracted with EtOAc (3×100 mL). The organic layers were combined, dried over Na₂SO₄, filtered, evaporated and purified on SiO₂ column (MeOH/dichloromethane/HOAc, 1:15:0.01) to afford the title compound (7.05 g, 87%). ESI-MS m/z: [M+H]⁺ calcd for C₁₁H₁₅NO₂S₂ 258.05. found 258.05.

### Example 128. Synthesis of N-Succinimidyl 4-(2-pyridyldithio)-4-methylpentanoate (246)

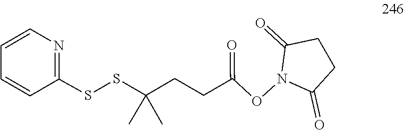 [SMILES: CC(C)(CCC(=O)ON1C(=O)CCC1=O)SSc1ccccn1]

4-(2-pyridyldithio)-4-methylpentanoic acid (2.0 g, 7.78 mmol) in dichloromethane (20 mL) was added N-hydroxysuccimide (1.10 g, 9.56 mmol) and EDC HCl (4.0 g, 20.8 mmol) and the mixture was stirred overnight, evaporated and purified on SiO₂ column (EtOAc/dichloromethane, 1:10) to afford the title compound (2.48 g, 90%). ESI-MS m/z: [M+Na]⁺ calcd for C₁₅H₁₈N₂O₄S₂ 377.07. found 377.08.

### Example 129. Synthesis of 1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethyl-N-(4-((S)-2-(4-methyl-4-(phenyldisulfanyl)pentanamido)propanamido)benzyl)piperidin-4-aminium (247)

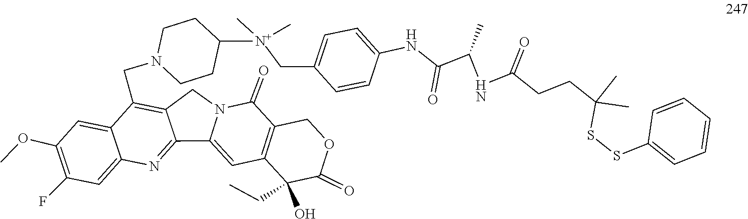 [SMILES: CC[C@@]1(O)C(=O)OCc2c1cc1n(c2=O)Cc2c-1nc1cc(F)c(OC)cc1c2CN1CCC([N+](C)(C)Cc2ccc(NC(=O)[C@H](C)NC(=O)CCC(C)(C)SSc3ccccc3)cc2)CC1]

Compound 246 (15 mg, 0.04 mmol) was dissolved in DMA (2 mL), to which compound 28 (56.8 mg, 0.08 mmol) and N, N-diisopropylethylamine (0.020 mL, 0.12 mmol) were added at 0° C. The reaction was warmed to r.t. and stirred for 2 hours, concentrated, and purified by preparative HPLC (acetonitrile/water containing formic acid) to give compound 247 (32 mg, 86% yield). ESI-MS m/z: M⁺ calcd for C₅₁H₆₀FN₆O₇S₂: 951.39. found 951.39.

### Example 130. Synthesis of (S)-4-ethyl-8-fluoro-4,9-dihydroxy-11-methyl-10-nitro-1H-pyrano[3′,4′:6,7]indolizino[1,2-b]quinoline-3,14(4H,12H)-dione (124)

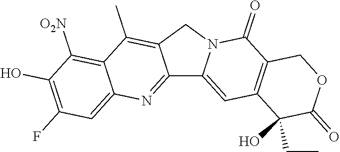 [SMILES: CC[C@@]1(O)C(=O)OCc2c1cc1n(c2=O)Cc2c-1nc1cc(F)c(O)c([N+](=O)[O-])c1c2C]

Compound 103 (451.1 mg, 1.139 mmol) in DCM (10 ml) were added HOAc (1 ml), Ac₂O (0.2 ml) and HNO₃ (conc., 0.3 ml, 4.665 mmol). The mixture was stirred for 3 h, diluted with water (10 ml), separated and the aqueous solution was extracted with DCM (3×25 ml). The organic layers were combined, dried over Na₂SO₄, filtered, and purified on short SiO₂ column eluted with MeOH/DCM (1:10) to afford the title compound (361.6 mg, 72% yield). ESI-MS m/z: (M+H)⁺ calcd for C₂₁H₁₇FN₃O₇: 442.3739. found 442.3810.

### Example 131. Synthesis of (S)-9-(bromomethoxy)-4-ethyl-8-fluoro-4-hydroxy-11-methyl-10-nitro-1H-pyrano[3′,4′:6,7]indolizino[1,2-b]quinoline-3,14(4H,12H)-dione (301)

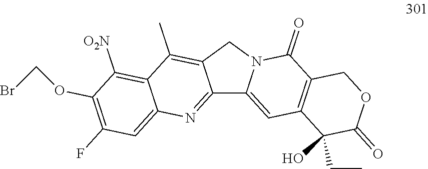 [SMILES: CC[C@@]1(O)C(=O)OCc2c1cc1n(c2=O)Cc2c-1nc1cc(F)c(OCBr)c([N+](=O)[O-])c1c2C]

Compound 124 (350.3 mg, 0.793 mmol), CH₂Br₂ (1 ml, 14.41 mmol) and NaHCO₃ (0.25 g, 2.97 mmol) in THF were stirred at 70° C. for 8 h. The mixture was concentrated and diluted with HCl (0.1 M, 8 ml) and H₂O (40 mL). The precipitated solid was filtered, dissolved in a small volume of (1:10) EtOAc/CH₂Cl₂, and purified by column chromatography using MeOH/CH₂Cl₂ (1:10-1:6) as eluent to afford the title compound (0.366 g, 86% yield). ESI-MS m/z: [M+H]⁺ calcd for C₂₂H₁₈BrFN₃O₇: 534.0313. found 534.0385.

### Example 132. Synthesis of (S)-8-ethyl-4-fluoro-8-hydroxy-15-methyl-11,14-dihydro-TH-oxazolo[4,5-f]pyrano[3′,4′:6,7]indolizino[1,2-b]quinoline-9,12(2H,8H)-dione (302)

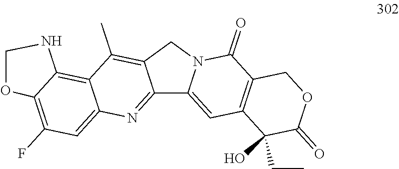 [SMILES: CC[C@@]1(O)C(=O)OCc2c1cc1n(c2=O)Cc2c-1nc1cc(F)c3c(c1c2C)NCO3]

To a stirred mixture of THF (10 ml) and a concentrated HCl solution (5 mL) at 0° C. was added (S)-9-(bromomethoxy)-4-ethyl-8-fluoro-4-hydroxy-11-methyl-10-nitro-H-pyrano[3′,4′:6,7]indolizino[1,2-b]quinoline-3,14(4H,12H)-dione (0.360 g, 0.675 mmol) in small portions, and the resulting clear solution was cooled to −10° C. after 15 min. To the reaction mixture was added SnCl₂ (0.384 g, 2.022 mmol) in small portions and the reaction mixture was allowed to warm to r. t., stirred for 1.5 h, and then cooled onto ice. The mixture was neutralized with slowly addition of NaHCO₃ to pH 5.5-6.0 on ice water, followed by refluxing at 70° C. for 6 h and concentrated in vacuo. The precipitate was filtered and washed with EtOH and Et₂O, and the aqueous filtrate was extracted with 10% MeOH/CH₂Cl₂. The organic solution was combined with the filtered precipitate dissolved in 30% MeOH/CH₂Cl₂, and then passed through a short SiO₂ pad eluted with 20% MeOH/CH₂Cl₂. The organic solvent was removed to afford the title compound (0.120 g, 42% yield in two steps), which was used in the next step without further purification. ESI-MS m/z: [M+H]⁺ calcd for C₂₂H₁₈FN₃O₅: 424.1309. found 424.1375.

### Example 133. Synthesis of (S)-tert-butyl (2-((2-(8-ethyl-4-fluoro-8-hydroxy-15-methyl-9,12-dioxo-2,8,9,11,12,14-hexahydro-1H-oxazolo[4,5-f]pyrano[3′,4′:6,7]indolizino[1,2-b]quinolin-1-yl)-2-oxoethyl)amino)-2-oxoethyl)carbamate (303)

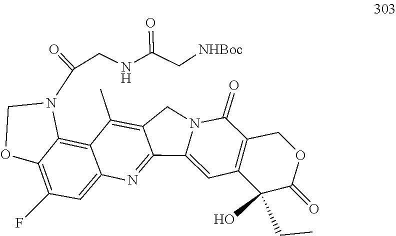 [SMILES: CC[C@@]1(O)C(=O)OCc2c1cc1n(c2=O)Cc2c-1nc1cc(F)c3c(c1c2C)N(C(=O)CNC(=O)CNC(=O)OC(C)(C)C)CO3]

(S)-8-ethyl-4-fluoro-8-hydroxy-15-methyl-11,14-dihydro-1H-oxazolo[4,5-f]pyrano[3′,4′:6,7]indolizino[1,2-b]quinoline-9,12(2H,8H)-dione, HCl salt (158.3 mg, 0.344 mmol), 2-(2-((tert-butoxycarbonyl)amino)acetamido)acetic acid (gly-gly-NHBoc) (103.9 mg, 0.447 mmol) and EDC (153.5 mg, 0.799 mmol) were stirred in DMA (10 ml) for 8 h. The mixture was concentrated and purified on SiO₂ column eluted with EtOAc/DCM (1:10-1:3) to afford the title compound (182.6 mg, 82% yield). ESI-MS m/z: (M+H)⁺ calcd for C₃₁H₃₃FN₅O₉: 638.2263; found 638.2295.

### Example 134. Synthesis of (S)-2-amino-N-(2-(8-ethyl-4-fluoro-8-hydroxy-15-methyl-9,12-dioxo-2,8,9,11,12,14-hexahydro-1H-oxazolo[4,5-f]pyrano[3′,4′:6,7]indolizino[1,2-b]quinolin-1-yl)-2-oxoethyl)acetamide, HCl salt (304)

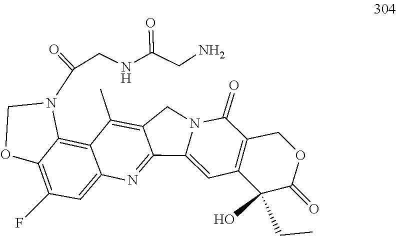 [SMILES: CC[C@@]1(O)C(=O)OCc2c1cc1n(c2=O)Cc2c-1nc1cc(F)c3c(c1c2C)N(C(=O)CNC(=O)CN)CO3]

(S)-tert-butyl (2-((2-(8-ethyl-4-fluoro-8-hydroxy-15-methyl-9,12-dioxo-2,8,9,11,12,14-hexahydro-1H-oxazolo[4,5-f]pyrano[3′,4′:6,7]indolizino[1,2-b]quinolin-1-yl)-2-oxoethyl)amino)-2-oxoethyl)carbamate (175.6 mg, 0.275 mmol) in the mixture of HCl concentrated solution (1 ml) and dioxane (4 ml) was stirred for 30 min. The mixture was diluted with toluene (5 ml), concentrated and co-evaporated with DCM/toluene (5:5 ml, 2 times) to afford the title compound for the next step without further purification (154.6 mg, 98% yield). ESI-MS m/z: (M+H)⁺ calcd for C₂₆H₂₅FN₅O₇: 538.1739. found 538.1780.

### Example 135. Synthesis of Compound 305

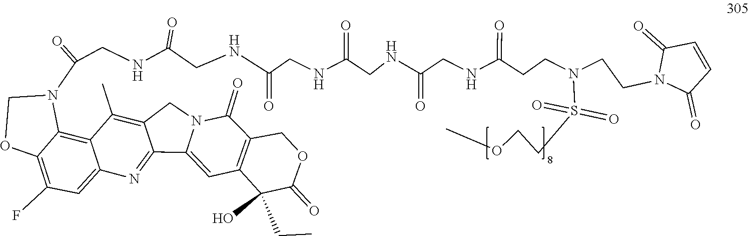

In a solution of compound 236 (83.2 mg, 0.102 mmol) and compound 274 (55.1 mg, 0.0960 mmol) in DMA (8 ml) was added EDC (95.5 mg, 0.497 mmol). The mixture was stirred overnight, concentrated and purified on SiO₂ column eluted with MeOH/DCM (1:6-1:3) to afford the compound 305 (103.3 mg, 81% yield). ESI-MS m/z: (M+H)⁺ calcd for C₅₈H₇₈FN₁₀O₂₃S: 1333.4947. found 1333.5015.

### Example 136. Synthesis of (R)-2-(4-(2,5-dioxo-2,5-dihydro-1H-pyrrol-1-yl)butanamido)-N1-(4-((2-((2-((S)-8-ethyl-4-fluoro-8-hydroxy-15-methyl-9,12-dioxo-2,8,9,11,12,14-hexahydro-1H-oxazolo[4,5-f]pyrano[3′,4′:6,7]indolizino[1,2-b]quinolin-1-yl)-2-oxoethyl)amino)-2-oxoethyl)amino)-4-oxobutyl)-N5-(2,5,8,11,14,17,20,23-octaoxapentacosan-25-yl)pentanediamide (306)

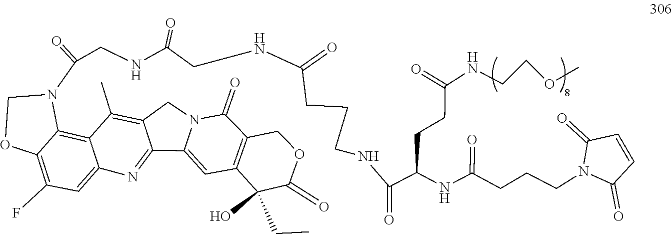

To a solution of compound 304 (47.3 mg, 0.088 mmol) and (S)-30-(4-(2,5-dioxo-2,5-dihydro-1H-pyrrol-1-yl)butanamido)-27,31-dioxo-2,5,8,11,14,17,20,23-octaoxa-26,32-diazahexatriacontan-36-oic acid (compound 6) (70.1 mg, 0.092 mmol) in DMF (5 mL), EDC (55 mg, 0.286 mmol) was added. The reaction was stirred for 8 hours. After concentration, the residue was purified by purified on SiO₂ column eluted with MeOH/DCM (1:6-1:3) to afford the compound 306 (89.3 mg, 79% yield). ESI-MS m/z: (M+H)⁺ calcd for C₆₀H₈₁FN₉O₂₁: 1282.5532. found 1282.5590.

### Example 137. Synthesis of 5-amino-4-(2-chloroacetyl)-2-methoxy-N-methylbenzamide (307)

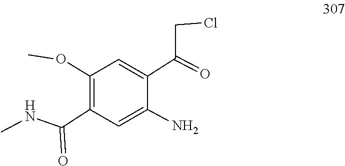 [SMILES: CNC(=O)c1cc(N)c(C(=O)CCl)cc1OC]

A solution of 5-amino-2-methoxy-N-methylbenzamide (5.00 g, 27.76 mmol) dissolved in dichloromethane (20 mL) was added dropwise to an ice water cooled boron trichloride (1 M in dichloromethane, 38.9 mL) solution. The reaction was stirred for 10 minutes and then chloroacetonitrile (3.2 g, 42.5 mmol) and aluminum trichloride (5.2 g, 38.9 mmol) were added. After the addition was completed, the reaction was warmed to r.t. and then refluxed overnight. The reaction mixture was then cooled to about 0° C., quenched with 2 M HCl (80 mL) and stirred at r.t. for 2 hours. Layers were separated and the aqueous phase was extracted with dichloromethane (3×80 mL). Combined organic phases were washed with water (100 mL), dried over sodium sulfate, filtered, concentrated, purified on a C-18 column, eluted with EtOH/H₂O (1:6 to 1:1) to give compound 307 (3.05 g, 43% yield) as a yellow solid. ESI-MS m/z: [M+H]⁺ calcd for C₁₁H₁₄ClN₂O₃: 257.0693. found 257.0725.

### Example 138. Synthesis of (S)-11-(chloromethyl)-4-ethyl-4-hydroxy-9-methoxy-N-methyl-3,14-dioxo-3,4,12,14-tetrahydro-1H-pyrano[3′,4′:6,7]indolizino[1,2-b]quinoline-8-carboxamide (308)

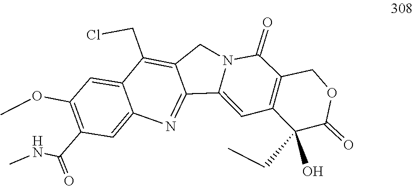 [SMILES: CC[C@@]1(O)C(=O)OCc2c1cc1n(c2=O)Cc2c-1nc1cc(C(=O)NC)c(OC)cc1c2CCl]

Compound 307 (0.59 g, 2.30 mmol) and compound 25 (0.57 g, 2.19 mmol) were dissolved in anhydrous toluene (40 mL), and p-toluenesulfonic acid (42 mg, 0.219 mmol) was added. The suspension was heated at reflux for 2 days and allowed to cool to r.t. After removal of about two-thirds of toluene, the residue was filtered and the filter cake was washed with dichloromethane, air-dried to give compound 308 (0.74 g, 70% yield) as a gray powdery solid. ESI-MS m/z: [M+H]⁺ calcd for C₂₄H₂₃ClN₃O₆: 484.1276. found 484.1220.

### Example 139. Synthesis of N-(4-((S)-2-((tert-butoxycarbonyl)amino)propanamido)benzyl)-1-(((S)-4-ethyl-4-hydroxy-9-methoxy-8-(methylcarbamoyl)-3,14-dioxo-3,4,12,14-tetrahydro-1H-pyrano[3′,4′:6,7]indolizino[1,2-b]quinolin-11-yl)methyl)-N,N-dimethylpiperidin-4-aminium, formic acid salt (309)

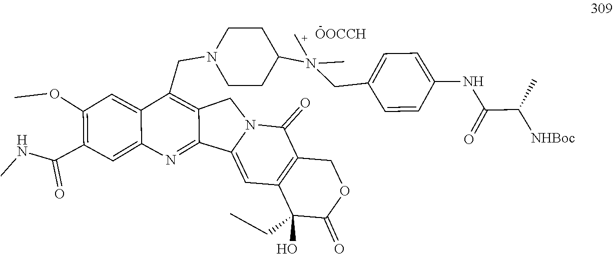

A mixture of compound 308 (238 mg, 0.49 mmol), compound 18 (200 mg, 0.49 mmol) in DMF (5 mL) was stirred at 0° C. for 30 minutes, then triethylamine (63 μL, 0.45 mmol) was added and the stirring was continued for 1 hour. The reaction was concentrated and purification by preparative HPLC (acetonitrile/water containing formic acid, Φ=5 cm, v=30 ml/min, 100% water to 50% water in 45 min) gave compound 309 (242 mg, 55% yield) as a yellow solid. ESI-MS m/z: M⁺ calcd for C₄₆H₅₈N₇O₉: 852.4291. found 852.4355.

### Example 140. Synthesis of N-(4-((S)-2-aminopropanamido)benzyl)-1-(((S)-4-ethyl-4-hydroxy-9-methoxy-8-(methylcarbamoyl)-3,14-dioxo-3,4,12,14-tetrahydro-1H-pyrano[3′,4′:6,7]-indolizino[1,2-b]quinolin-11-yl)methyl)-N,N-dimethylpiperidin-4-aminium, trifluoroacetic acid salt (310)

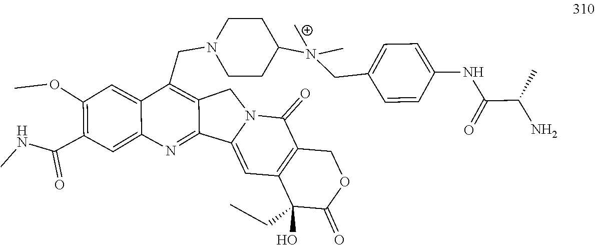

Compound 309 (95 mg, 0.111 mmol) was dissolved in a mixture of dichloromethane and trifluoroacetic acid (2 mL/6 mL), and stirred at r.t. for 30 minutes. The mixture was diluted with toluene (10 ml), then concentrated and dried on a vacuum pump to give compound 310 (108 mg, 100% yield) as a yellow solid. ESI-MS m/z: M⁺ calcd for C₄₁H₅₀N₇O₇: 752.3766. found 752.3710.

### Example 141. Synthesis of N-(4-((30S,38S)-30-(4-(2,5-dioxo-2,5-dihydro-1H-pyrrol-1-yl)butanamido)-38-methyl-27,31,36-trioxo-2,5,8,11,14,17,20,23-octaoxa-26,32,37-triazanonatriacontanamido)benzyl)-1-(((S)-4-ethyl-4-hydroxy-9-methoxy-8-(methylcarbamoyl)-3,14-dioxo-3,4,12,14-tetrahydro-1H-pyrano[3′,4′:6,7]indolizino[1,2-b]quinolin-11-yl)methyl)-N,N-dimethylpiperidin-4-aminium formate (311)

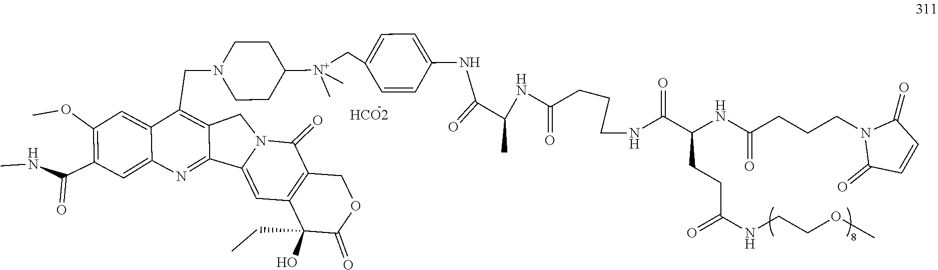

Compound 310 (60 mg, 0.061 mmol) and compound 7 (60 mg, 0.064 mmol) were dissolved in DMF (5 mL), cooled to about 0° C., and then N, N-diisopropylethylamine (21 μL, 0.12 mmol) was added. The reaction was warmed to r.t. and stirred for 2 hours, concentrated, and purified by preparative HPLC (acetonitrile/water containing formic acid, Φ=3 cm, v=20 ml/min, 100% water to 50% water in 45 min) to give compound 281 (38.5 mg, 41% yield). ESI-MS m/z: M⁺ calcd for C₇₅H₁₀₆N₁₁O₂₁: 1496.7559. found 1496.7595.

### Example 142. Synthesis of (2R,3S)-2,3-bis(((benzyloxy)carbonyl)amino)succinic acid (312)

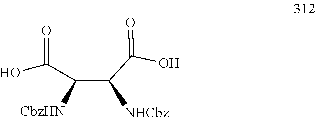 [SMILES: O=C(N[C@H](C(=O)O)[C@@H](NC(=O)OCc1ccccc1)C(=O)O)OCc1ccccc1]

To a solution of (2R,3S)-2,3-diaminosuccinic acid (4.03 g, 27.30 mmol) in the mixture of THF (250 ml) and NaH₂PO₄ (0.1 M, 250 ml, pH 8.0) was added benzyl carbonochloridate (15.0 g, 88.23 mmol) in 4 portions in 2 h. The mixture was stirred for another 6 h, concentrated and purified on SiO₂ column eluted with H₂O/CH₃CN (1:9) containing 1% formic acid to afford the title compound (8.63 g, 75% yield). MS ESI m/z calcd for C₂₀H₂₁N₂O₈ [M+H]⁺ 417.12, found 417.50.

### Example 143. Synthesis of (2R,3S)-bis(2,5-dioxopyrrolidin-1-yl) 2,3-bis(((benzyloxy)-carbonyl)amino)succinate

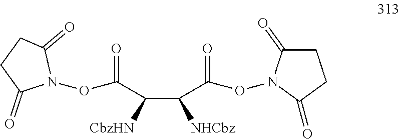 [SMILES: O=C(N[C@H](C(=O)ON1C(=O)CCC1=O)[C@@H](NC(=O)OCc1ccccc1)C(=O)ON1C(=O)CCC1=O)OCc1ccccc1]

To a solution of (2R,3S)-2,3-bis(((benzyloxy)carbonyl)amino)succinic acid (4.25 g, 10.21 mmol) in the mixture of DMA (70 ml) was added NHS (3.60 g, 31.30 mmol) and EDC (7.00 g, 36.65 mmol). The mixture was stirred for overnight, concentrated and purified on SiO₂ column eluted with EtOAc/CH₂Cl₂ (1:6) to afford the title compound (5.48 g, 88% yield). MS ESI m/z calcd for C₂₈H₂₇N₄O₁₂ [M+H]⁺ 611.15, found 611.45.

### Example 144. Synthesis of di-tert-butyl 4,4′-(((2R,3S)-2,3-bis(((benzyloxy)carbonyl)-amino)succinyl)bis(azanediyl))dibutanoate

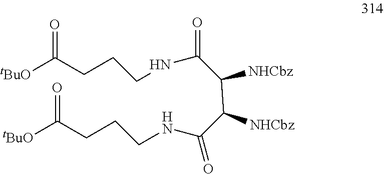 [SMILES: CC(C)(C)OC(=O)CCCNC(=O)[C@@H](NC(=O)OCc1ccccc1)[C@@H](NC(=O)OCc1ccccc1)C(=O)NCCCC(=O)OC(C)(C)C]

To a solution of (2R,3S)-2,3-bis(((benzyloxy)carbonyl)amino)succinic acid (4.25 g, 10.21 mmol) in the mixture of DMA (70 ml) was added tert-butyl 4-aminobutanoate (3.25 g, 20.42 mmol) and EDC (7.00 g, 36.65 mmol). The mixture was stirred for overnight, concentrated and purified on SiO₂ column eluted with EtOAc/CH₂Cl₂ (1:10) to afford the title compound (6.50 g, 91% yield). MS ESI m/z calcd for C₃₆H₅₁N₄O₁₀ [M+H]⁺ 699.35, found 699.55.

### Example 145. Synthesis of di-tert-butyl 4,4′-(((2R,3S)-2,3-diaminosuccinyl)-bis(azanediyl))dibutanoate

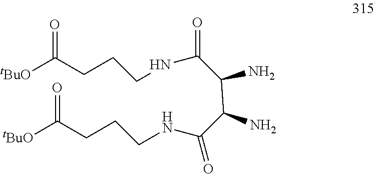 [SMILES: CC(C)(C)OC(=O)CCCNC(=O)[C@@H](N)[C@@H](N)C(=O)NCCCC(=O)OC(C)(C)C]

To a solution of di-tert-butyl 4,4′-(((2R,3S)-2,3-bis(((benzyloxy)carbonyl)amino)-succinyl)bis(azanediyl))dibutanoate (2.50 g, 3.58 mmol) in MeOH (100 mL) was added 10% Pd/C (0.30 g, 50% wet), the mixture was stirred under hydrogen atmosphere at room temperature for 18 h. Then the Pd/C was removed by filtration through celite and the filter bed was washed with MeOH (˜70 ml). The filtrate was concentrated to afford the product as yellow foam which was used in the next step without further purification (1.54 g, 100% yield). ESI: m/z: calcd for C₂₀H₃₉N₂O₆ [M+H]⁺: 431.28, found 431.50.

### Example 146. Synthesis of di-tert-butyl 4,4′-(((2R,3S)-2,3-bis(3-(2,5-dioxo-2,5-dihydro-1H-pyrrol-1-yl)propanamido)succinyl)bis(azanediyl))dibutanoate

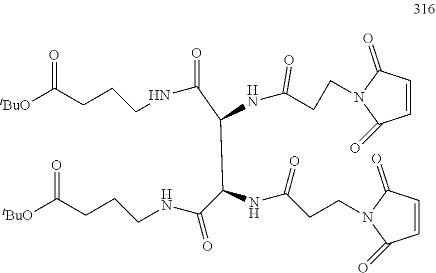 [SMILES: CC(C)(C)OC(=O)CCCNC(=O)[C@@H](NC(=O)CCN1C(=O)C=CC1=O)[C@@H](NC(=O)CCN1C(=O)C=CC1=O)C(=O)NCCCC(=O)OC(C)(C)C]

To a solution of 3-(2,5-dioxo-2,5-dihydro-1H-pyrrol-1-yl)propanoic acid (1.25 g, 7.39 mmol) in the mixture of DMA (60 ml) was added di-tert-butyl 4,4′-(((2R,3S)-2,3-diaminosuccinyl)-bis(azanediyl))dibutanoate (1.54 g, ˜3.57 mmol) and EDC (2.40 g, 12.56 mmol). The mixture was stirred for overnight, concentrated and purified on SiO₂ column eluted with EtOAc/CH₂Cl₂ (1:10) to afford the title compound (2.35 g, 90% yield). MS ESI m/z calcd for C₃₄H₄₉N₆O₁₂ [M+H]⁺ 733.33, found 733.60.

### Example 147. Synthesis of 4,4′-(((2R,3S)-2,3-bis(3-(2,5-dioxo-2,5-dihydro-1H-pyrrol-1-yl)propanamido)succinyl)bis(azanediyl))dibutanoic acid

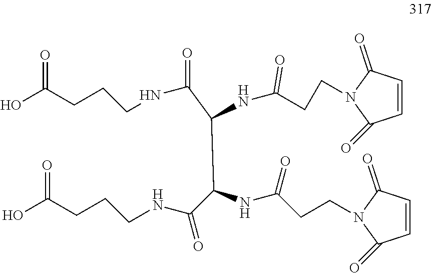 [SMILES: O=C(O)CCCNC(=O)[C@@H](NC(=O)CCN1C(=O)C=CC1=O)[C@@H](NC(=O)CCN1C(=O)C=CC1=O)C(=O)NCCCC(=O)O]

To a stirred solution of di-tert-butyl 4,4′-(((2R,3S)-2,3-bis(3-(2,5-dioxo-2,5-dihydro-1H-pyrrol-1-yl)propanamido)succinyl)bis(azanediyl))dibutanoate (2.30 g, 3.14 mmol) in 1,4-dioxane (20 ml) was added HCl (36%, 7.0 ml). The mixture was stirred for 30 min, diluted with toluene (20 ml), concentrated and purified on SiO₂ column eluted with MeOH/CH₂Cl₂ (1:10 to 1:4) to afford the title compound (1.69 g, 86% yield). MS ESI m/z calcd for C₂₆H₃₃N₆O₁₂ [M+H]⁺ 621.21, found 621.70.

### Example 148. Synthesis of di-tert-butyl 4,4′-(((2R,3S)-2,3-bis(2-(2,5-dioxo-2,5-dihydro-1H-pyrrol-1-yl)acetamido)succinyl)bis(azanediyl))dibutanoate

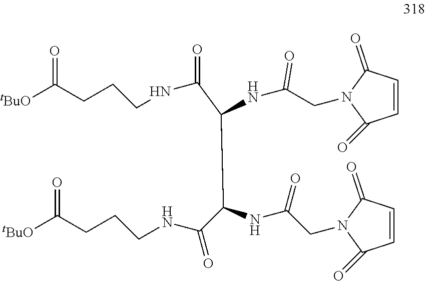 [SMILES: CC(C)(C)OC(=O)CCCNC(=O)[C@@H](NC(=O)CN1C(=O)C=CC1=O)[C@@H](NC(=O)CN1C(=O)C=CC1=O)C(=O)NCCCC(=O)OC(C)(C)C]

To a solution of 2-(2,5-dioxo-2,5-dihydro-1H-pyrrol-1-yl)acetic acid (1.12 g, 7.22 mmol) in the mixture of DMA (60 ml) was added di-tert-butyl 4,4′-(((2R,3S)-2,3-diaminosuccinyl)-bis(azanediyl))dibutanoate (1.54 g, ˜3.58 mmol) and EDC (2.40 g, 12.56 mmol). The mixture was stirred for overnight, concentrated and purified on SiO₂ column eluted with EtOAc/CH₂Cl₂ (1:10) to afford the title compound (2.29 g, 91% yield). MS ESI m/z calcd for C₃₂H₄₅N₆O₁₂ [M+H]⁺ 704.30, found 704.60.

### Example 149. Synthesis of 4,4′-(((2R,3S)-2,3-bis(2-(2,5-dioxo-2,5-dihydro-1H-pyrrol-1-yl)acetamido)succinyl)bis(azanediyl))dibutanoic acid

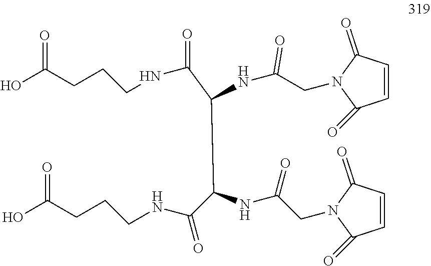 [SMILES: O=C(O)CCCNC(=O)[C@@H](NC(=O)CN1C(=O)C=CC1=O)[C@@H](NC(=O)CN1C(=O)C=CC1=O)C(=O)NCCCC(=O)O]

To a stirred solution of di-tert-butyl 4,4′-(((2R,3S)-2,3-bis(2-(2,5-dioxo-2,5-dihydro-1H-pyrrol-1-yl)acetamido)succinyl)bis(azanediyl))dibutanoate (2.20 g, 3.12 mmol) in 1,4-dioxane (20 ml) was added HCl (36%, 7.0 ml). The mixture was stirred for 30 min, diluted with toluene (20 ml), concentrated and purified on SiO₂ column eluted with MeOH/CH₂Cl₂ (1:10 to 1:4) to afford the title compound (1.69 g, 86% yield). MS ESI m/z calcd for C₂₄H₂₉N₆O₁₂ [M+H]⁺ 593.18, found 593.40.

### Example 150. Synthesis of bis(2,5-dioxopyrrolidin-1-yl) 4,4′-(((2R,3S)-2,3-bis(2-(2,5-dioxo-2,5-dihydro-1H-pyrrol-1-yl)acetamido)succinyl)bis(azanediyl))dibutanoate

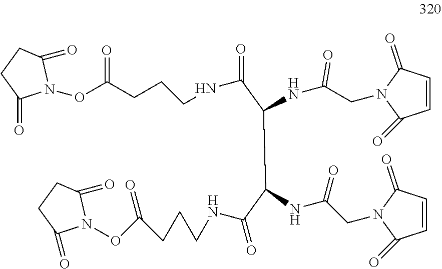 [SMILES: O=C(CN1C(=O)C=CC1=O)N[C@H](C(=O)NCCCC(=O)ON1C(=O)CCC1=O)[C@@H](NC(=O)CN1C(=O)C=CC1=O)C(=O)NCCCC(=O)ON1C(=O)CCC1=O]

To a solution of 4,4′-(((2R,3S)-2,3-bis(2-(2,5-dioxo-2,5-dihydro-1H-pyrrol-1-yl)acetamido)succinyl)bis(azanediyl))dibutanoic acid (1.10 g, 1.85 mmol) in the mixture of DMA (30 ml) was added NHS (1-hydroxypyrrolidine-2,5-dione) (0.55 g, 4.78 mmol) and EDC (1.25 g, 6.54 mmol). The mixture was stirred for overnight, concentrated and purified on SiO₂ column eluted with EtOAc/CH₂Cl₂ (1:10) to afford the title compound (1.30 g, 90% yield). MS ESI m/z calcd for C₃₂H₃₅N₈O₁₆ [M+H]⁺ 787.21, found 787.60.

### Example 151. Synthesis of (2S,3S)-2,3-bis(2,5-dioxo-2,5-dihydro-1H-pyrrol-1-yl)succinic acid

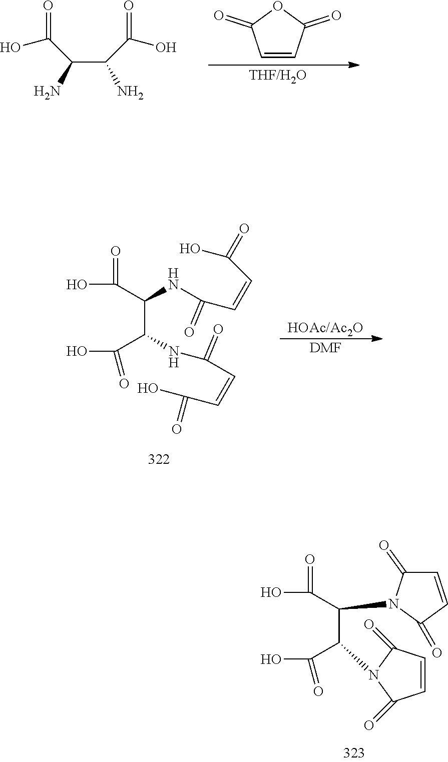

(2R,3R)-2,3-diaminosuccinic acid (5.00 g, 33.77 mmol) in the mixture of THF/H₂O/DIPEA (125 ml/125 ml/2 ml) was added maleic anhydride (6.68 g, 68.21 mmol).

The mixture was stirred for overnight, evaporated to afforded (2S,3S)-2,3-bis((Z)-3-carboxyacrylamido)succinic acid (11.05 g, 99% yield) as a white solid. MS ESI m/z calcd for C₁₂H₁₃N₂O₁₀ [M+H]⁺ 345.05, found 345.35.

(2S,3S)-2,3-bis((Z)-3-carboxyacrylamido)succinic acid (11.05 g, 33.43 mmol) in a mixture solution of HOAc (70 ml), DMF (10 ml) and toluene (50 ml) was added acetic anhydride (30 ml). The mixture was stirred for 2 h, reflux with Dean-Stark Trap at 100° C. for 6 h, concentrated, co-evaporated with EtOH (2×40 ml) and toluene (2×40 ml), and purified on SiO₂ column eluted with H₂O/CH₃CN (1:10) to afford the title compound (8.10 g, 78% yield). MS ESI m/z calcd for C₁₂H₉N₂O₈[M+H]⁺ 309.03, found 309.50.

### Example 152. Synthesis of (2S,3S)-bis(2,5-dioxopyrrolidin-1-yl) 2,3-bis(2,5-dioxo-2,5-dihydro-1H-pyrrol-1-yl)succinate

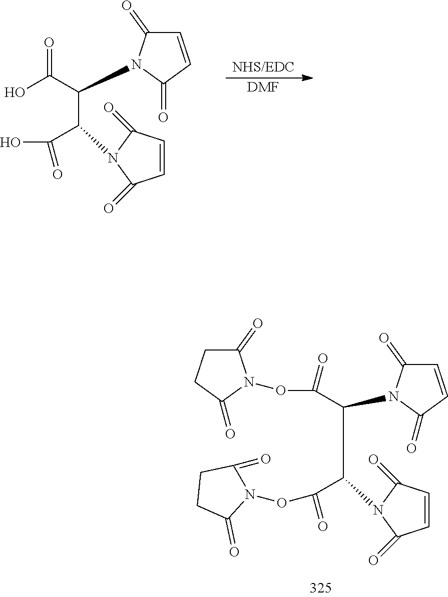

To a solution of (2S,3S)-2,3-bis(2,5-dioxo-2,5-dihydro-1H-pyrrol-1-yl)succinic acid (4.00 g, 12.98 mmol) in the mixture of DMF (70 ml) was added NHS (3.60 g, 31.30 mmol) and EDC (7.00 g, 36.65 mmol). The mixture was stirred for overnight, concentrated and purified on SiO₂ column eluted with EtOAc/CH₂Cl₂ (1:6) to afford the title compound (5.79 g, 89% yield, ˜96% pure by HPLC). MS ESI m/z calcd for C₂₀H₁₅N₄O₁₂ [M+H]⁺ 503.06, found 503.60.

### Example 153. Synthesis of 4-(((benzyloxy)carbonyl)amino)butanoic acid

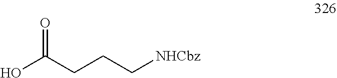 [SMILES: O=C(O)CCCNC(=O)OCc1ccccc1]

To a solution of NaOH (23.3 g, 0.58 mol, 2.0 eq) in water (140 mL) was added 4-aminobutanoic acid (30.0 g, 0.29 mol, 1.0 eq) and THF (60 mL) at −20° C., then CbzCl (54 mL, 0.38 mol, 1.3 eq) in THF (57 mL) was added dropwise. The reaction mixture was stirred at room temperature for 4 h, then concentrated and washed with EtOAc (4×100 mL). Concentrated hydrochloric acid was added to the aqueous solution until pH 3 was reached. The solution was extracted with EA (4×150 mL, 2×100 mL), and the combined organic phase was washed with brine, dried over anhydrous sodium sulfate, filtered, and concentrated to give the title compound as a white solid (48.3 g, 70.3%). ESI m/z: calcd for C₁₂H₁₆NO₄ [M+H]⁺ 238.1, found 238.1.

### Example 154. Synthesis of tert-butyl 4-(((benzyloxy)carbonyl)amino)butanoate

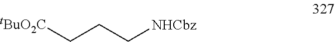 [SMILES: CC(C)(C)OC(=O)CCCNC(=O)OCc1ccccc1]

To a solution of 4-(((benzyloxy)carbonyl)amino)butanoic acid (48.0 g, 0.2 mol, 1.0 eq.) and t-BuOH (58.0 mL, 0.6 mol, 3.0 eq.) in anhydrous dichloromethane (480 mL) were added DCC (50.0 g, 0.24 mol, 1.2 eq.) and DMAP (2.5 g, 0.02 mol, 0.1 eq.) at 0° C., and the mixture then was warmed to room temperature and stirred overnight. The solid was filtered off and the filtrate was concentrated, then diluted with EtOAc (400 mL) and washed with 5% NaHCO₃ solution and brine, dried over anhydrous sodium sulfate, filtered, then concentrated. The residue was purified by SiO₂ column chromatography (PE/EtOAc=5:1) to give the title compound as a colorless oil (32.8 g, 55.1%). ESI m/z: calcd for C₁₆H₂₄NO₄ [M+H]⁺ 294.2, found 294.2.

### Example 155. Synthesis of tert-butyl 4-aminobutanoate

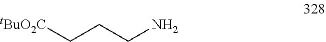 [SMILES: CC(C)(C)OC(=O)CCCN]

To a solution of tert-butyl 4-(((benzyloxy)carbonyl)amino)butanoate (29.0 g, 0.099 mol, 1.0 eq.) in MeOH (100 mL) was added Pd/C (2.9 g, 10% Pd/C, 50% wet) in a hydrogenation bottle. The mixture was shaken under 1 atm H₂ overnight. The reaction mixture was filtered, and the filtrate was concentrated to give the title compound as a colorless oil (13.8 g, 83.7% yield). ESI m/z: calcd for C₈H₁₈NO₂ [M+H]⁺ 160.1, found 160.1.

### Example 156. Synthesis of 11-(benzyloxy)-11-oxoundecanoic acid

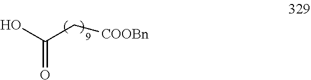

To a solution of undecanedioic acid (1.73 g, 8 mmol) in DMF (30 mL) were added K₂CO₃ (1.1 g, 8 mmol) and BnBr (1.36 g, 8 mmol). The mixture was stirred at r.t. overnight, then concentrated and purified by column chromatography (PE/EtOAc) to afford the title compound (1.1 g, 45% yield). ESI m/z: calcd for C₁₈H₂₇O₄ [M+H]⁺: 307.18, found 307.15.

### Example 157. Synthesis of 3-(2-(2-(dibenzylamino)ethoxy)ethoxy)propanoic acid

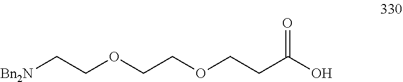

To a solution of tert-butyl 3-(2-(2-(dibenzylamino)ethoxy)ethoxy)propanoate (2.00 g, 4.84 mmol) in DCM (5 mL) was added HCO₂H (5 mL). The reaction was stirred at room temperature overnight, then concentrated to dryness and co-evaporated twice with DCM, and the residue was placed on a pump to give the title compound (1.72 g, ˜100% yield). ESI m/z calcd for C₂₁H₂₇NO₄ [M+H]⁺: 358.19, found 358.19.

### Example 158. Synthesis of tert-butyl 2-benzyl-11-oxo-1-phenyl-5,8,15,18-tetraoxa-2,12-diazahenicosan-21-oate

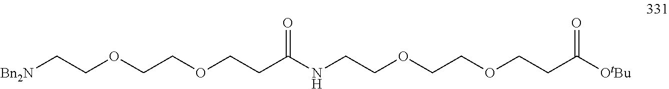

To a solution of 3-(2-(2-(dibenzylamino)ethoxy)ethoxy)propanoic acid (1.12 g, 4.83 mmol) and tert-butyl 3-(2-(2-aminoethoxy)ethoxy)propanoate (1.72 g, 4.83 mmol) in DCM (30 mL) were added HATU (1.83 g, 4.83 mmol) and TEA (0.68 mL, 4.83 mmol) at 0° C. The reaction was warmed to r.t. and stirred for 1 h, then diluted with 50 mL DCM and poured into a separatory funnel containing 50 mL of water. The organic phase was separated, and washed with brine (50 mL), dried over anhydrous Na₂SO₄, filtered and concentrated. The residue was purified by column chromatography (MeOH/DCM) to afford the title compound (2.21 g, 80% yield). ESI m/z calcd for C₃₂H₄₈N₂O₇ [M+H]⁺: 573.35, found 573.35.

### Example 159. Synthesis of tert-butyl 1-amino-9-oxo-3,6,13,16-tetraoxa-10-azanonadecan-19-oate

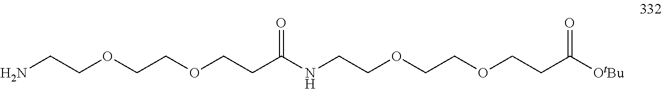 [SMILES: CC(C)(C)OC(=O)CCOCCOCCNC(=O)CCOCCOCCN]

To a solution of tert-butyl 2-benzyl-11-oxo-1-phenyl-5,8,15,18-tetraoxa-2,12-diazahenicosan-21-oate (2.21 g, 3.86 mmol) in MeOH (20 mL) was added Pd/C (10 wt %, 0.2 g) in a hydrogenation bottle. The mixture was stirred under 1 atm H₂ overnight, filtered through Celite (filter aid), and the filtrate was concentrated to afford the title compound (1.5 g, ˜100% yield). ESI m/z calcd for C₁₈H₃₆N₂O₇ [M+H]⁺: 393.25, found 393.25

### Example 160. Synthesis of 31-benzyl 1-tert-butyl 11,21-dioxo-4,7,14,17-tetraoxa-10,20-diazahentriacontane-1,31-dioate

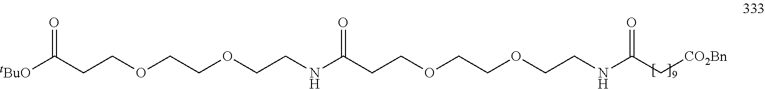

To a solution of tert-butyl 1-amino-9-oxo-3,6,13,16-tetraoxa-10-azanonadecan-19-oate (1.50 g, 3.86 mmol) and 11-(benzyloxy)-11-oxoundecanoic acid (1.10 g, 3.6 mmol) in DCM (50 mL) were added HATU (1.48 g, 3.9 mmol) and TEA (0.55 mL, 3.9 mmol) at 0° C. The reaction mixture was stirred at r.t. for 1 h, then diluted with 50 mL DCM and poured into a separatory funnel containing 50 mL of water. The organic phase was separated, washed with brine (50 mL), dried over anhydrous Na₂SO₄, filtered and concentrated. The residue was purified by column chromatography (MeOH/DCM) to afford the title compound (1.50 g, 61% yield). ESI m/z calcd for C₃₆H₆₁N₂O₁₀ [M+H]⁺: 681.42, found 681.42.

### Example 161. Synthesis of 3,13,23-trioxo-1-phenyl-2,17,20,27,30-pentaoxa-14,24-diazatritriacontan-33-oic acid

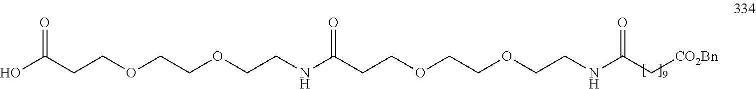

To a solution of 31-benzyl 1-tert-butyl 11,21-dioxo-4,7,14,17-tetraoxa-10,20-diazahentriacontane-1,31-dioate (1.50 g, 2.2 mmol) in DCM (1 mL) was added TFA (3 mL). The reaction was stirred at room temperature for 1 h, then concentrated to dryness and co-evaporated twice with DCM, and the residue was placed on a pump to give the title compound (0.09 g, 2.2 mmol, crude product). ESI m/z: calcd for C₃₂H₅₃N₂O₁₀ [M+H]⁺: 625.36, found 625.35.

### Example 162. Synthesis of(S)-39-(((benzyloxy)carbonyl)amino)-3,13,23,33-tetraoxo-1-phenyl-2,17,20,27,30-pentaoxa-14,24,34-triazatetracontan-40-oic acid

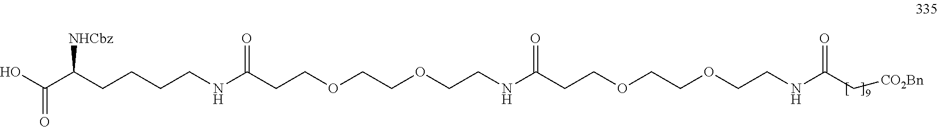

To a solution of 3,13,23-trioxo-1-phenyl-2,17,20,27,30-pentaoxa-14,24-diazatritriacontan-33-oic acid (1.50 g, 2.20 mmol) and Z-Lys-OH (0.62 g, 2.20 mmol) in DCM (50 mL) were added HATU (0.84 g, 2.20 mmol) and TEA (0.31 mL, 2.20 mmol) at 0° C. The reaction mixture was stirred at r.t. for 1 h, then diluted with 50 mL DCM and poured into a separatory funnel containing 100 mL of water. The organic phase was separated, and washed with brine (100 mL), dried over anhydrous Na₂SO₄, filtered and concentrated. The residue was purified by column chromatography (MeOH/DCM) to afford the title compound (1.00 g, 53% yield). ESI m/z calcd for C₄₆H₇₁N₄O₁₃ [M+H]⁺: 887.49, found 887.50.

### Example 163. Synthesis of di-tert-butyl 3,3′-((oxybis(ethane-2,1-diyl))bis(oxy)) dipropanoate

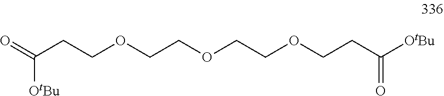 [SMILES: CC(C)(C)OC(=O)CCOCCOCCOCCC(=O)OC(C)(C)C]

To a solution of diethylene glycol (20 g, 0.188 mol) in THF (200 mL) was added Na (0.43 g, 0.018 mol). After stirring at r.t. for 1 h, tert-butyl acrylate (48 g, 0.376 mol) was added and the reaction mixture was stirred at r.t. for 2 days. The reaction was concentrated under vacuum and purified by column chromatography to afford the title compound (34 g, 50% yield). ESI m/z calcd for C₁₈H₃₅O₇[M+H]⁺: 363.23, found 363.23.

### Example 164. Synthesis of 3,3′-((oxybis(ethane-2,1-diyl))bis(oxy))dipropanoic acid

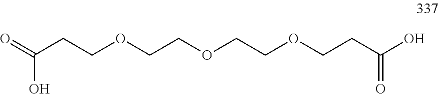 [SMILES: O=C(O)CCOCCOCCOCCC(=O)O]

Di-tert-butyl 3,3′-((oxybis(ethane-2,1-diyl))bis(oxy))dipropanoate (34 g, 0.093 mol) was dissolved in formic acid (100 mL) at room temperature and stirred overnight. The reaction was concentrated under vacuum to afford the title compound. ESI m/z calcd for C₁₀H₁₉O₇[M+H]⁺: 251.11, found 251.11.

### Example 165. Synthesis of 2,2-dimethyl-4,14,24-trioxo-3,7,10,17,20,27,30,33-octaoxa-13,23-diazahexatriacontan-36-oic acid

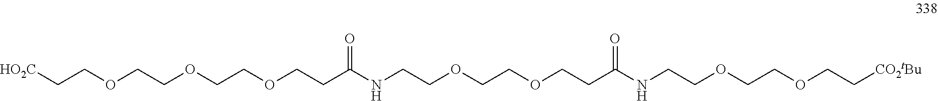 [SMILES: CC(C)(C)OC(=O)CCOCCOCCNC(=O)CCOCCOCCNC(=O)CCOCCOCCOCCC(=O)O]

To a solution of tert-butyl 1-amino-9-oxo-3,6,13,16-tetraoxa-10-azanonadecan-19-oate (1.50 g, 3.82 mmol) and 3,3′-((oxybis(ethane-2,1-diyl))bis(oxy))dipropanoic acid (1.90 g, 7.64 mmol) in DMF (10 mL) were added HATU (1.45 g, 3.82 mmol) and DIPEA (0.66 mL, 3.82 mmol) at 0° C. The reaction mixture was warmed to r.t. and stirred for 1 h, then diluted with DCM (80 mL), washed with water (10 mL), dried over sodium sulfate, filtered, concentrated and purified by silica gel column chromatography to afford the title compound as a colorless liquid (1.75 g, 75% yield). ESI m/z calcd for C₂₈H₅₃N₂O₁₃ [M+H]⁺: 625.35, found 625.35.

### Example 166 Synthesis of 1-tert-butyl 33-(2,5-dioxopyrrolidin-1-yl) 11,21-dioxo-4,7,14,17,24,27,30-heptaoxa-10,20-diazatritriacontane-1,33-dioate

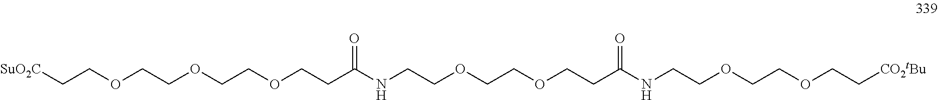

To a solution of 2,2-dimethyl-4,14,24-trioxo-3,7,10,17,20,27,30,33-octaoxa-13,23-diazahexatriacontan-36-oic acid (1.75 g, 2.8 mmol) in DCM (20 mL) were added EDCI (1.07 g, 5.6 mmol) and NHS (0.64 g, 5.6 mmol) at 0° C. The reaction was warmed to room temperature and stirred overnight, then diluted with DCM (80 mL), washed with water (10 mL), dried over sodium sulfate, filtered and concentrated under vacuum to afford the title compound (2.00 g, ˜100% yield). ESI m/z calcd for C₃₂H₅₆N₃O₁₅ [M+H]⁺: 722.36, found 722.36.

### Example 167. Synthesis of (S)-42-(((benzyloxy)carbonyl)amino)-2,2-dimethyl-4,14,24,36-tetraoxo-3,7,10,17,20,27,30,33-octaoxa-13,23,37-triazatritetracontan-43-oic acid

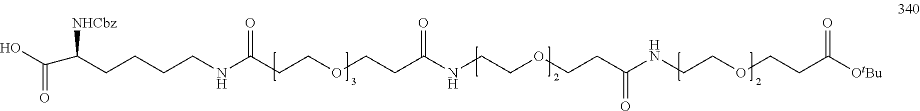

To a solution of N-α-Cbz-L-lysine (1.17 g, 4.2 mmol) in water (10 mL) was added sodium bicarbonate (0.47 g, 5.6 mmol), and the reaction mixture was cooled to 5° C., and 1-tert-butyl 33-(2,5-dioxopyrrolidin-1-yl) 11,21-dioxo-4,7,14,17,24,27,30-heptaoxa-10,20-diazatritriacontane-1,33-dioate (2.00 g, 2.8 mmol) dissolved in 1,4-Dioxane (10 mL) was added. The reaction was warmed to r.t. and stirred for 1 h, then acidified to pH 3 by addition of 1 N HCl, extracted with DCM (50 mL×3). The organic extracts were washed with water (20 mL), dried over sodium sulfate, filtered and concentrated to afford the title product (2.3 g, 92% yield). ESI m/z calcd for C₄₂H₇₁N₄O₁₆ [M+H]⁺: 887.48, found 887.48.

### Example 168. Synthesis of (S)-43-benzyl 1-tert-butyl 7-(((benzyloxy)carbonyl)amino)-6,13,23,33-tetraoxo-16,19,26,29-tetraoxa-5,12,22,32-tetraazatritetracontane-1,43-dioate

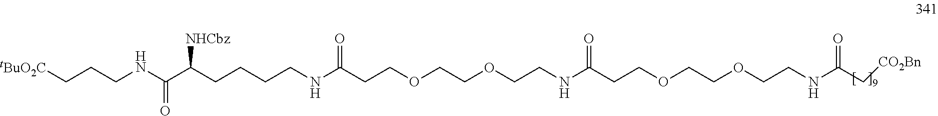

(S)-39-(((benzyloxy)carbonyl)amino)-3,13,23,33-tetraoxo-1-phenyl-2,17,20,27,30-pentaoxa-14,24,34-triazatetracontan-40-oic acid (200 mg, 0.225 mmol) was dissolved in DMF (5 mL) and cooled to 0° C., tert-butyl 4-Aminobutanoate (71.8 mg, 0.45 mmol) and EDC (86.2 mg, 0.45 mmol) were added in sequence. The reaction was warmed to r.t. and stirred overnight, poured into ice-water, and extraction with DCM (3×10 mL). The combined organic phase was washed with water (5 mL), brine (5 mL), dried over anhydrous Na₂SO₄, filtered and concentrated to give the title compound (231 mg, 100% yield). ESI m/z calcd for C₈₄H₈₆N₅O₁₄ [M+H]⁺:1028.61, found: 1028.61.

### Example 169. Synthesis of (7S,10R,11S,14S)-di-tert-butyl 10,11-bis(((benzyloxy)-carbonyl)amino)-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioate (342)

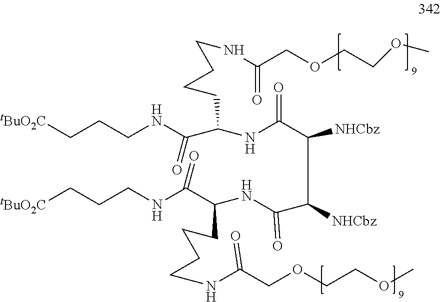

A mixture of (S)-tert-butyl 37-(((benzyloxy)carbonyl)amino)-31,38-dioxo-2,5,8,11,14,17, 20,23,26,29-decaoxa-32,39-diazatritetracontan-43-oate (5.98 g, 6.73 mmol) and Pd/C (10 wt %, 0.6 g) in methanol (30 mL) was hydrogenated under 1 atm H₂ pressure overnight and then filtered through Celite (filter aid). The filtrate was concentrated and re-dissolved in THF (60 mL), (2R,3S)-2,3-bis(((benzyloxy)carbonyl)amino)succinic acid (1.01 g, 2.42 mmol) and HOBt (817 mg, 6.05 mmol) were added at 0° C. DCC (1.25 g, 6.05 mmol) and DIPEA (2.1 mL, 12.10 mmol) were added in sequence. The reaction was stirred at r.t. overnight, then diluted with EtOAc (400 mL), and washed with 0.1N HCl, saturated sodium bicarbonate and brine, dried over anhydrous Na₂SO₄, filtered, concentrated and purified by SiO₂ column chromatography (24:1 DCM/MeOH) to give the title compound (5.65 g, 49% yield). MS ESI m/z calcd for C₉₀H₁₅₄N₈O₃₄ [M+H]⁺ 1892.06. found 1892.60.

### Example 170. Synthesis of (7S,10R,11S,14S)-di-tert-butyl 10,11-diamino-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioate (343)

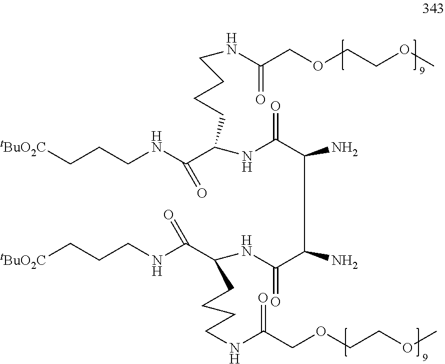

A mixture of (7S,10R,11S,14S)-di-tert-butyl 10,11-bis(((benzyloxy)-carbonyl)amino)-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioate (3.71 g, 1.96 mmol) and Pd/C (10 wt %, 0.40 g) in methanol (50 mL) was hydrogenated under 1 atm H₂ pressure overnight and then filtered through Celite (filter aid). The filtrate was concentrated to afford the title compound (3.18 g, 100% yield). MS ESI m/z calcd for C₇₄H₁₄₂N₈O₃₀ [M+H]⁺ 1623.98. found 1624.50.

### Example 171. Synthesis of (7S,10R,11S,14S)-10,11-bis(4-(2,5-dioxo-2,5-dihydro-1H-pyrrol-1-yl)butanamido)-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioic acid (344)

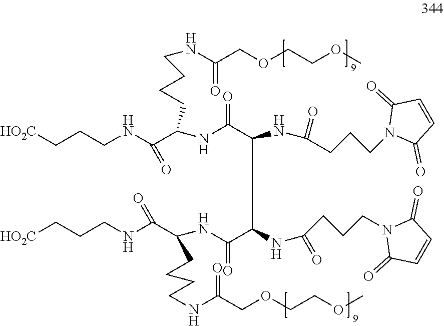

To a solution of (7S,10R,11S,14S)-di-tert-butyl 10,11-diamino-6,9,12,15-tetraoxo-7,14-bis(31-oxo-2,5,8,11,14,17,20,23,26,29-decaoxa-32-azahexatriacontan-36-yl)-5,8,13,16-tetraazaicosane-1,20-dioate (315 mg, 0.194 mmol) in DMA (10 mL) were added EDC (150 mg, 0.785 mmol) and 4-maleido-butanoic acid (72 mg, 0.57 mmol). The mixture was stirred at room temperature for 12 h, concentrated and purified by SiO₂ column chromatography (1:4 MeOH/DCM) to give an oil (329 mg, 87% yield), which was dissolved in dichloromethane (25 mL) and treated with TFA (5 mL) at r.t. for 1 h, and then concentrated to afford the title compound (309 mg, 99% yield). MS ESI m/z calcd for C₈₂H₁₄₀N₁₀O₃₆ [M+H]⁺ 1841.94, found 1842.50.

### Example 172. Synthesis of (S)-11-(5-(tert-butoxy)-2-((tert-butoxycarbonyl)amino)-5-oxopentanamido)undecanoic acid (345)

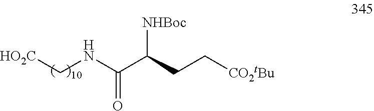

To a solution of Boc-Glu(OtBu)-OH (0.50 g, 1.65 mmol) in DMF (10 mL) were added HATU (0.69 g, 1.82 mmol) and TEA (0.26 mL, 1.82 mmol). After stirring for 30 min, a solution of 11-aminoundecanoic acid (0.33 g, 1.65 mmol) in DMF (10 mL) was added and the reaction was stirred at r.t. for 1 h, then poured into a separatory funnel containing 200 mL of 1N HCl and extracted with DCM (3×50 mL). The organic phase was washed once with 100 mL of brine, then dried over anhydrous Na₂SO₄, filtered and concentrated. The residue was purified by column chromatography (MeOH/DCM) to afford the title compound (1.0 g, >100% yield). ESI: m/z: calcd for C₂₅H₄₇N₂O₇ [M+H]⁺: 487.33, found 487.34.

### Example 173. Synthesis of (S)-11-(2-amino-4-carboxybutanamido)undecanoic acid

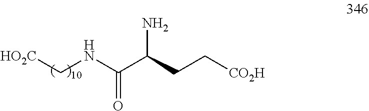

To a solution of (S)-11-(5-(tert-butoxy)-2-((tert-butoxycarbonyl)amino)-5-oxopentanamido)undecanoic acid (1.0 g, ˜2.05 mmol) in DCM (20 mL) was added TFA (5 mL). The reaction was stirred at room temperature for 30 min, then concentrated to dryness and dried twice with DCM. Finally, placed on a vacuum pump give the title compound (0.68 g, ˜2.06 mmol, ˜100% yield). ESI: m/z: calcd for C₁₆H₃₁N₂O₅ [M+H]⁺: 331.22, found 331.22.

### Example 174. Synthesis of Compound 347

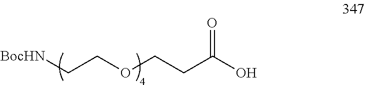

In a 500 mL flask, H₂N-PEG₄-CH₂CH₂CO₂H (3.0 g, 11.3 mmol, 1.0 eq) and K₂CO₃ (4.7 g, 33.93 mmol, 3.0 eq) were dissolved in 50 mL of water, and cooled over an ice water bath.

Boc₂O (3.2 g, 14.7 mmol, 1.3) in 50 mL of THF was added dropwise. The reaction was allowed to warm to r.t. and stirred overnight. The reaction mixture was adjusted to pH 4-5 with 1N KHSO₄ and extracted with DCM (200 mL×1, 100 mL×3), washed with water (500 mL×1), and brine (500 mL×1), dried over anhydrous sodium sulfate, and concentrated. The residue was dissolved in a small amount of DCM and then loaded on a silica gel column, eluted with 2-4% MeOH/DCM, and the fractions were combined and concentrated to give 3.8 g of colorless oil compound 347 (yield 93%). ESI m/z calcd. for C₁₆H₃₂NO₈ [M+H]⁺: 366.2, found: 366.2.

### Example 175. Synthesis of Compound 348

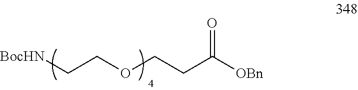

In a 50 mL single-necked flask, BocHN-PEG₄-CH₂CH₂CO₂H (0.81 g, 2.22 mmol, 1.0 eq), K₂CO₃ (0.92 g, 6.66 mmol, 3.0 eq) and NaI (0.033 g, 0.222 mmol, 0.1 eq) were mixed in 10 mL of DMF, cooled over an ice water bath, and BnBr (0.57 g, 3.33 mmol, 1.5 eq) was added dropwise, and the mixture was warmed to r.t. and stirred overnight. The reaction mixture was diluted with 100 mL of water, extracted with DCM (100 mL×2), washed with water (200 mL×1), and brine (200 mL×1), dry over anhydrous sodium sulfate, and concentrated. The residue was dissolved in a small amount of DCM, loaded on silica gel column, eluted with is 70-90% EA/PE to give 0.69 g of colorless oil compound 348 (69% yield). ESI m/z calcd. for C₂₃H₃₈NO₈ [M+H]⁺: 446.3, found: 446.3.

### Example 176. Synthesis of Compound 349

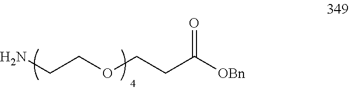

A solution of BocHN-PEG₄-CH₂CH₂CO₂Bn (0.69 g, 1.5 mmol, 1.0 eq) in 6 mL of DCM and 3 mL of TFA was stirred at r.t. for 30 min. The solvents were removed and the residue was co-evaporated with DCM for three times, placed on high vacuum pump. The crude product was used directly in the next reaction. ESI m/z calcd. for C₁₈H₃₀NO₆ [M+H]⁺: 356.2, found: 356.2.

### Example 177. Synthesis of Compound 350

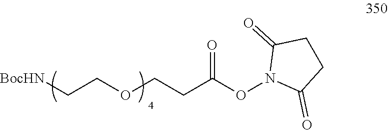

To a solution of BocHN-PEG₄-CH₂CH₂CO₂H (3.8 g, 10.4 mmol, 1.0 eq) in 50 mL of dry DCM, NHS (1.4 g, 12.5 mmol, 1.2 eq) and EDC (10.0g, 52.0 mmol, 5.0 eq) were added. The reaction was stirred at r.t. overnight and then washed with water (50 mL×2), brine (100 mL×1), dried over anhydrous sodium sulfate, and concentrated. The crude product was used directly in the next step. ESI m/z calcd. for C₂₀H₃₅N₂O₁₀ [M+H]⁺: 463.2, found: 463.2.

### Example 178. Synthesis of Compound 351

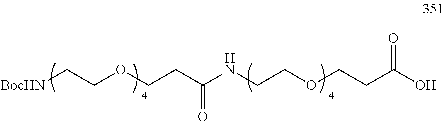

In a 300 mL flask, H₂N-PEG₄-CH₂CH₂CO₂H (2.8 g, 10.4 mmol, 1.0 eq) and K₂CO₃ (4.3 g, 31.2 mmol, 3.0 eq) were dissolved in 40 mL of water, cooled over an ice water bath, and the above crude NHS ester solution (3.8 g, 10.4 mmol, 1.0 eq) in 40 mL of THF was added dropwise, and the mixture was warmed to r.t. and stirred overnight. The reaction mixture was adjusted to pH 4-5 using 1N KHSO₄, extracted with DCM (150 mL×1, 100 mL×2), washed with water (200 mL×1), and brine (200 mL×1), dried over anhydrous sodium sulfate, and concentrated. The residue was dissolved in small amount of DCM, and the loaded on a silica gel column, eluted with 4-6% MeOH/DCM to give a colorless oil (5.18 g, 81% yield). ESI m/z calcd. for C₂₇H₅₃N₂O₁₃ [M+H]⁺: 613.3, found: 613.3.

### Example 179. Synthesis of Compound 352

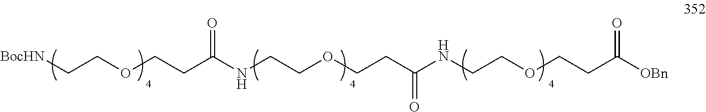

H₂N-PEG₄-CH₂CH₂CO₂Bn (crude product from the previous step) dissolved in 3 mL of DMF, cooled over ice/water bath, DIPEA (0.78 g, 6.0 mmol, 4.0 eq) was added dropwise, and followed by addition of a solution of compound 22 (0.93 g, 1.5 mmol, 1.0 eq) in 7 mL of DMF and HATU (1.72 g, 4.5 mmol, 3.0 eq). The reaction was stirred over the ice bath for 2 hours, and diluted with 100 mL of water, extracted with DCM (100 mL×3), washed with 1N KHSO₄ (200 mL×1), saturated sodium bicarbonate (200 mL×1), and brine (200 mL×1), dried over anhydrous sodium sulfate, and concentrated. The residue was dissolved in a small amount of DCM, loaded on a silica gel column, and eluted 0-5% MeOH/DCM. Fractions were combined and concentrated to give 1.0 g of light-yellow oil (71% yield). ESI m/z calcd. for C₄₅H₈₀N₃O₁₈ [M+H]⁺: 950.5, found: 950.5.

### Example 180. Synthesis of (S)-tert-butyl 34-(((benzyloxy)carbonyl)amino)-28,35-dioxo-2,5,8,11,14,17,20,23,26-nonaoxa-29,36-diazatetracontan-40-oate (196)

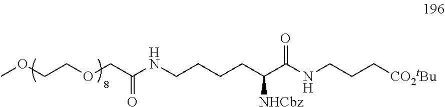

A mixture of tert-butyl 4-aminobutanoate (1.03 g, 6.12 mmol) and (S)-34-(((benzyl-oxy)carbonyl)amino)-28-oxo-2,5,8,11,14,17,20,23,26-nonaoxa-29-azapentatriacontan-35-oic acid (3.91 g, 5.56 mmol) in DMF (18 mL) at 0° C., HATU (2.32 g, 6.12 mmol) and TEA (1.2 mL, 8.34 mmol) were added in sequence. The reaction was stirred for 1 h, then diluted with water (300 mL), and extracted with ethyl acetate (3×250 mL). The organic solution was washed with brine, dried over anhydrous sodium sulfate, filtered, concentrated and purified by silica gel column chromatography (32:1 dichloromethane/methanol) to give the title compound (5.10 g, 99% yield). ESI MS m/z 846.50 ([M+H]⁺).

### Example 181. Synthesis of (S)-tert-butyl 34-amino-28,35-dioxo-2,5,8,11,14,17,20,23,26-nonaoxa-29,36-diazatetracontan-40-oate (197)

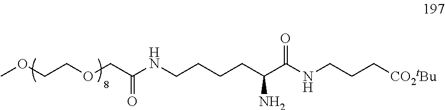

Compound 210 (1.0 g, 1.18 mmol) and Pd/C (10 wt %, 0.10 g) were added in a hydrogenation bottle having methanol (50 mL). The mixture was shaken for 2 h, filtered through Celite (filter aid), and the filtrate was concentrated to afford compound 197 (0.93 g, yield>100%). ESI MS m/z 712.50 ([M+H]⁺).

### Example 182. Synthesis of (S)-tert-butyl 34-(4-(2,5-dioxo-2,5-dihydro-1H-pyrrol-1-yl)butanamido)-28,35-dioxo-2,5,8,11,14,17,20,23,26-nonaoxa-29,36-diazatetracontan-40-oate (353)

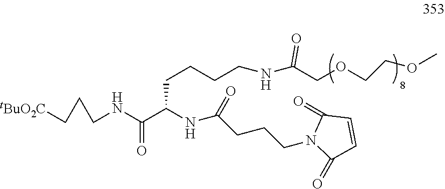

To a solution of compound 197 (0.93 g, 1.18 mmol) in 95% EtOH (50 mL) and NaH₂PO₄ solution (0.1 M, pH 5.0, 10 mL), N-succinimidyl 4-maleimido-butyrate (0.50 g, 1.77 mmol, 1.5 eq) was added. The mixture was stirred overnight, then concentrated and diluted with water (50 mL) and extracted with dichloromethane (80 mL×3), dried over anhydrous sodium sulfate, filtered, concentrated and purified by silica gel column chromatography (25:1 dichloromethane/methanol) to give the title compound as a light yellow oil (0.82 g, 80%). ESI MS m/z 877.52 ([M+H]⁺).

### Example 183. Synthesis of (S)-34-(4-(2,5-dioxo-2,5-dihydro-1H-pyrrol-1-yl)butanamido)-28,35-dioxo-2,5,8,11,14,17,20,23,26-nonaoxa-29,36-diazatetracontan-40-oic acid (354)

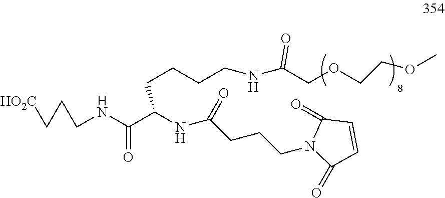

Compound 353 (0.82 g, 0.94 mmol) was dissolved in HCOOH (50 mL) and stirred at room temperature for 1 hour. The reaction mixture was concentrated and co-evaporated with toluene twice, and the residue was placed on a vacuum pump to give compound 354 (0.80 g, crude product). ESI MS m/z 820.45 ([M+H]⁺).

### Example 184. Synthesis of (S)-2,5-dioxopyrrolidin-1-yl 34-(4-(2,5-dioxo-2,5-dihydro-1H-pyrrol-1-yl)butanamido)-28,35-dioxo-2,5,8,11,14,17,20,23,26-nonaoxa-29,36-diazatetracontan-40-oate (355)

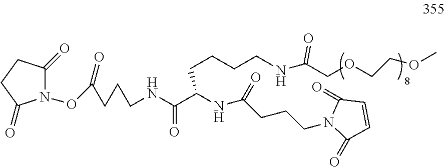

To a solution of compound 213 (0.80 g, crude, 0.94 mmol) in DMA (5.0 mL), NHS (0.12 g, 1.03 mmol) and EDC·HCl (0.27 g, 1.41 mmol) were added, and the reaction was stirred at r.t. for 2 h, then diluted with water (15 mL) and extracted with ethyl acetate (3×10 mL). The combined organic phase was washed with brine (10 mL), dried over anhydrous sodium sulfate, filtered and concentrated. The residue was purified by silica gel column (10-50% ethyl acetate/petroleum ether) to give a colorless oil compound (0.67 g, 78% yield). ESI MS m/z 918.55 ([M+H]⁺).

### Example 185. Synthesis of tert-butyl (2-(2,5-dioxo-2,5-dihydro-1H-pyrrol-1-yl)ethyl)carbamate (356)

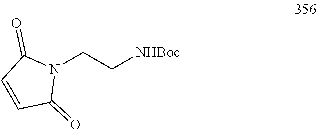 [SMILES: CC(C)(C)OC(=O)NCCN1C(=O)C=CC1=O]

A mixture of N-Boc-ethylenediamine (5.6 mL, 35.4 mmol, 1.1 eq.) and saturated NaHCO₃ (60 mL) was cooled to 0° C., to which N-methoxycarbonyl maleimide (5.00 g, 32.2 mmol, 1.0 eq.) was added in portions. After stirring at 0° C. for 30 min, the reaction was warmed to r.t. and stirred for 1 h. The precipitate was collected by filtration and washed with cold water, then dissolved in ethyl acetate and washed with brine, dried over anhydrous sodium sulfate and concentrated to give a white solid (6.69 g, 87% yield). ESI MS m/z 241.12 ([M+H]⁺).

### Example 186. Synthesis of tert-butyl (2-(1,3-dioxo-3a,4,7,7a-tetrahydro-1H-4,7-epoxyisoindol-2(3H)-yl)ethyl)carbamate (357)

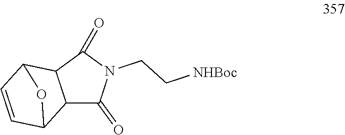 [SMILES: CC(C)(C)OC(=O)NCCN1C(=O)C2C3C=CC(O3)C2C1=O]

In a high pressure tube, a solution of compound 356 (6.00 g, 25.0 mmol), furan (18.0 mL) in toluene (120 mL) was heated to reflux and stirred for 16 h. The colorless solution turned yellow during reaction. The mixture was then cooled to r.t. and concentrated. The resulting white solid was triturated with ethyl ether to give compound 357 (6.5 g, 84% yield). ESI MS m/z 309.13 ([M+H]⁺).

### Example 187. Synthesis of 2-(2-aminoethyl)-3a,4,7,7a-tetrahydro-1H-4,7-epoxyisoindole-1,3(2H)-dione hydrochloride (358)

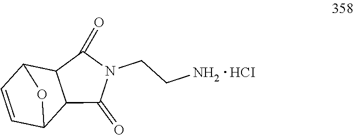

A solution of compound 357 (9.93 g, 32.2 mmol) in dioxane (15 mL) was treated with concentrated HCl (15 mL) at r.t. for 3 h. The reaction was concentrated and the resulting solid was collected by filtration, with washing of the filter cake with ethyl acetate. The solid was dried in an oven (50° C.) overnight to give compound 217 (6.94 g, 88% yield). ESI MS m/z 206.05 ([M+H]⁺).

### Example 188. Synthesis of Compound 359

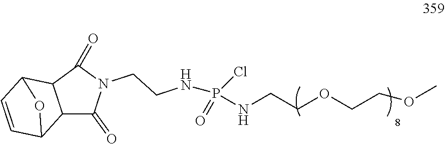

To a solution of compound 358 (1.22 g, 5 mmol) in THF (10 mL) and CH₃CN (10 ml) at −10° C., POCl₃ (0.47 mL, 5 mmol) was added. After stirring for 10 min., 2,5,8,11,14,17,20,23,26-nonaoxaoctacosan-28-amine (2.14 g, 5 mmol) was added, followed by DIPEA (0.87 mL, 5 mmol).

The reaction was warmed to 0° C. and stirred for 3 h, and then concentrated. The residue was diluted with dichloromethane (10 mL) and filtered over Celite, the filtrate was concentrated in vacuo to afford crude compound (˜3.7 g, ˜50% pure) which was used in the next step directly. ESI MS m/z 716.29 ([M+H]⁺).

### Example 189. Synthesis of Compound 360

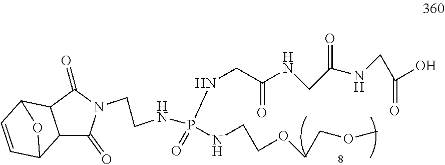

To a solution of 2-(2-(2-aminoacetamido)acetamido)acetic acid (gly-gly-gly, 0.501 g, 2.644 mmol) in CH₃CN (20 ml) and DIPEA (0.87 ml, 5 mmol), compound 359 (1.00 g, 50% pure, ˜0.699 mmol) was added. The mixture was stirred at 40° C. for 6 h, concentrated and purified by preparative HPLC (acetonitrile/water containing formic acid, Φ=5 cm, v=30 ml/min, 70% water to 25% water in 45 min) to give compound 360 (321.5 mg, ˜53% yield). ESI-MS m/z: (M+H)⁺ calcd for C₃₅H₆₂N₆O₁₇P: 869.3910. found 869.3995.

### Example 190. Synthesis of Compound 361

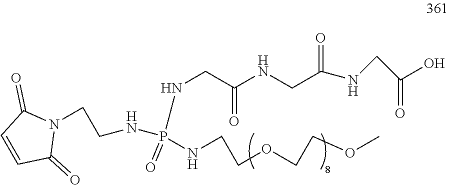

A solution of compound 360 (160.1 mg, 0.184 mmol) in DMA (10 ml) and toluene (10 ml) was refluxed for 8 h, concentrated and purified by preparative C-18 HPLC (acetonitrile/water containing 1% formic acid, Φ=3 cm, v=20 ml/min, 70% water to 25% water in 45 min) to give compound 361 (125.5 mg, 85% yield) after lyophilization. ESI-MS m/z: (M+H)⁺ calcd for C₃₅H₆₂N₆O₁₇P: 801.3648. found 801.3725.

### Example 190. Synthesis of Compound 362

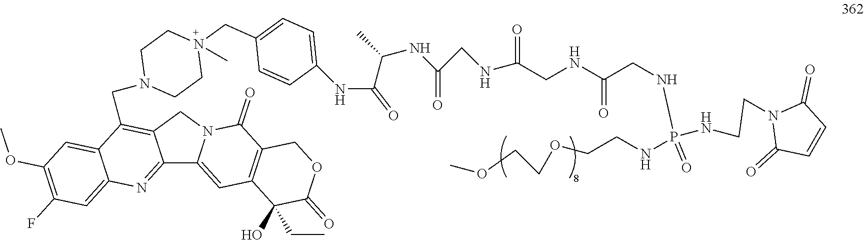

To a solution of compound 40 (50 mg, 0.064 mmol) and compound 361 (51.5 mg, 0.064 mmol) in DMF (5 mL), EDC (99.5 mg, 0.517 mmol) and N, N-diisopropylethylamine (45 μL, 0.26 mmol) were added. The reaction was stirred at r.t for 6 h, concentrated, and purified by preparative C-18 HPLC (acetonitrile/water containing 0.5% formic acid, Φ=3 cm, v=20 ml/min, 70% water to 25% water in 45 min) to give compound 41 (66.7 mg, 71% yield). ESI-MS m/z: M⁺ calcd for C₄₅H₄₉FN₇O₉: 1467.6607. found 1467.6675.

### Example 191. Synthesis of 14-(benzyloxy)-14-oxotetradecanoic acid (363)

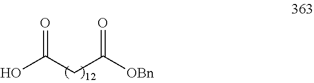

To a solution of tetradecanedioic acid (2.06 g, 8 mmol) in DMF (30 mL), K₂CO₃ (1.1 g, 8 mmol) and BnBr (1.36 g, 8 mmol) were added. The mixture was stirred at r.t. overnight, then concentrated and purified by column chromatography (ethyl acetate/petroleum ether) to afford the title compound 363 (1.2 g, 45% yield). ESI MS m/z 349.23 ([M+H]⁺).

### Example 192. Synthesis of tert-butyl 3-(2-(2-(2-hydroxyethoxy)ethoxy)ethoxy)propanoate (364)

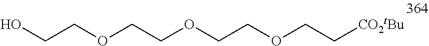 [SMILES: CC(C)(C)OC(=O)CCOCCOCCOCCO]

To a solution of 2,2′-(ethane-1,2-diylbis(oxy))diethanol (55.0 mL, 410.75 mmol, 3.0 eq.) in anhydrous THF (200 mL), sodium (0.1 g) was added. The mixture was stirred until Na disappeared and then tert-butyl acrylate (20.0 mL, 137.79 mmol, 1.0 eq.) was added dropwise. The mixture was stirred overnight and then quenched by HCl solution (20.0 mL, 1N) at 0° C. THF was removed by rotary evaporation, brine (300 mL) was added and the resulting mixture was extracted with ethyl acetate (3×100 mL). The organic layers were washed with brine (3×300 mL), dried over anhydrous sodium sulfate, filtered and concentrated to afford a colorless oil of the title compound (30.20 g, 79.0% yield), which was used without further purification. MS ESI m/z 278.17 ([M+H]⁺).

### Example 193. Synthesis of tert-butyl 3-(2-(2-(2-(tosyloxy)ethoxy)ethoxy)ethoxy) propanoate (365)

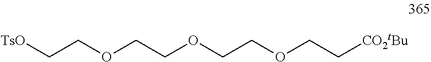 [SMILES: Cc1ccc(S(=O)(=O)OCCOCCOCCOCCC(=O)OC(C)(C)C)cc1]

To a solution of tert-butyl 3-(2-(2-(2-hydroxyethoxy)ethoxy)ethoxy) propanoate (30.20 g, 108.5 mmol, 1.0 eq.) and TsCl (41.37 g, 217.0 mmol, 2.0 eq.) in anhydrous DCM (220 mL) at 0° C., TEA (30.0 mL, 217.0 mmol, 2.0 eq.) was added. The mixture was stirred at room temperature overnight, and then washed with water (3×300 mL) and brine (300 mL), dried over anhydrous sodium sulfate, filtered, concentrated and purified by silica gel column chromatography (3:1 hexanes/ethyl acetate) to give a colorless oil (39.4 g, 84.0% yield). MS ESI m/z 433.28 ([M+H]⁺).

### Example 194. Synthesis of tert-butyl 3-(2-(2-(2-azidoethoxy)ethoxy)ethoxy) propanoate (366)

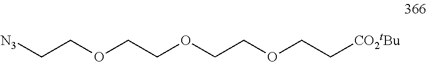 [SMILES: CC(C)(C)OC(=O)CCOCCOCCOCCN=[N+]=[N-]]

To a solution of tert-butyl 3-(2-(2-(2-(tosyloxy)ethoxy)ethoxy)ethoxy) propanoate (39.4 g, 91.1 mmol, 1.0 eq.) in anhydrous DMF (100 mL), NaN₃ (20.67 g, 316.6 mmol, 3.5 eq.) was added.

The mixture was stirred at room temperature overnight. Water (500 mL) was added and extracted with ethyl acetate (3×300 mL). The combined organic layers were washed with water (3×900 mL) and brine (900 mL), dried over anhydrous sodium sulfate, filtered, concentrated and purified by silica gel column chromatography (5:1 hexanes/ethyl acetate) to give a light-yellow oil (23.8 g, 85.53% yield). MS ESI m/z 326.2 ([M+Na]⁺).

### Example 195. Synthesis of tert-butyl 3-(2-(2-(2-aminoethoxy)ethoxy)ethoxy)propanoate (367)

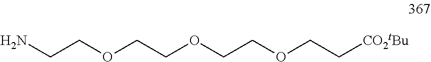 [SMILES: CC(C)(C)OC(=O)CCOCCOCCOCCN]

Raney-Ni (7.5 g, suspended in water) was washed with water (three times) and isopropyl alcohol (three times) and mixed with compound 366 (5.0 g, 16.5 mmol) in isopropyl alcohol. The mixture was stirred under a H₂ balloon at r.t. for 16 h and then filtered over a Celite pad, with washing of the pad with isopropyl alcohol. The filtrate was concentrated and purified by column chromatography (5-25% methanol/dichloromethane) to give a light-yellow oil (2.60 g, 57% yield). MS ESI m/z 279.19 ([M+H]⁺).

### Example 196. Synthesis of 27-benzyl 1-tert-butyl 14-oxo-4,7,10-trioxa-13-azaheptacosane-1,27-dioate (368)

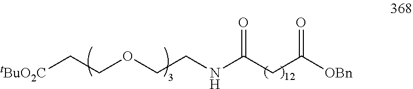

To a solution of compound 363 (2.60 g, 9.35 mmol) and compound 367 (3.91 g, 11.2 mmol) in dichloromethane (50 mL), EDC·HCl (2.15 g, 11.2 mmol) and DIPEA (3.6 mL, 20.6 mmol) were added. The reaction mixture was stirred at r.t. for 1 h, then diluted with 50 mL dichloromethane and poured into a separatory funnel containing 50 mL of water. The organic phase was separated, washed with brine (50 mL), dried over anhydrous sodium sulfate, filtered and concentrated. The residue was purified by column chromatography (0-10% methanol/dichloromethane) to afford the title compound 368 (4.94 g, 87% yield). ESI m/z 608.40 ([M+H]⁺).

### Example 197. Synthesis of 3,16-dioxo-1-phenyl-2,20,23,26-tetraoxa-17-azanonacosan-29-oic acid (369)

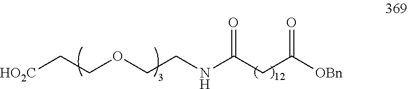

To a solution of compound 368 (4.94 g, 8.14 mmol) in dichloromethane (20 mL), TFA (20 mL) was added. The reaction was stirred at room temperature for 1 h, then concentrated to dryness and co-evaporated twice with dichloromethane, and the residue was placed on a pump to give compound 369 (4.50 g, crude product). ESI MS m/z 552.35 ([M+H]⁺).

### Example 198. Synthesis of 40-benzyl 1-tert-butyl 14,27-dioxo-4,7,10,17,20,23-hexaoxa-13,26-diazatetracontane-1,40-dioate (370)

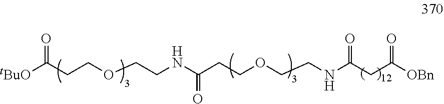

To a solution of compound 369 (4.50 g, crude, 8.14 mmol) and compound 367 (1.95 g, 7.00 mmol) in dichloromethane (50 mL), EDC·HCl (1.56 g, 8.14 mmol) and DIPEA (2.7 mL, 15.4 mmol) were added. The reaction mixture was stirred at r.t. for 1 h, then diluted with 50 mL dichloromethane and poured into a separatory funnel containing 50 mL of water. The organic phase was separated, washed with brine (50 mL), dried over anhydrous sodium sulfate, filtered and concentrated. The residue was purified by column chromatography (0-10% methanol/dichloromethane) to afford the title compound 370 (5.22 g, 92% yield). ESI m/z 811.52 ([M+H]⁺).

### Example 199. Synthesis of 3,16,29-trioxo-1-phenyl-2,20,23,26,33,36,39-heptaoxa-17,30-diazadotetracontan-42-oic acid (371)

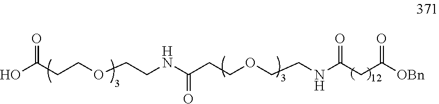

To a solution of compound 370 (5.22 g, 6.44 mmol) in dichloromethane (20 mL), TFA (5 mL) was added. The reaction was stirred at room temperature for 1 h, then concentrated to dryness and co-evaporated twice with dichloromethane, and the residue was placed on a pump to give compound 370 (4.90 g, crude product). ESI MS m/z 755.46 ([M+H]⁺).

### Example 200. Synthesis of 40-benzyl 1-(2,5-dioxopyrrolidin-1-yl) 14,27-dioxo-4,7,10,17,20,23-hexaoxa-13,26-diazatetracontane-1,40-dioate (372)

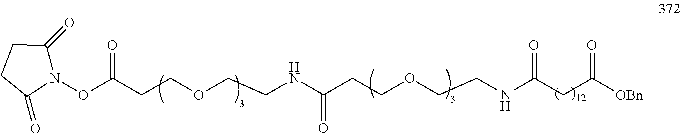

To a solution of compound 371 (4.90 g, crude, 6.44 mmol) in dichloromethane (30 mL), NHS (0.81 g, 7.08 mmol), EDC·HCl (1.85 g, 9.66 mmol), and DIPEA (2.8 mL, 16.1 mmol) were added. The reaction mixture was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified by silica gel column (10-50% ethyl acetate/petroleum ether) to give a colorless oil 372 (4.90 g, 90% yield). ESI MS m/z 852.48 ([M+H]⁺).

### Example 201. Synthesis of 1-((2,5-dioxopyrrolidin-1-yl)oxy)-1,14,27-trioxo-4,7,10,17,20,23-hexaoxa-13,26-diazatetracontan-40-oic acid (373)

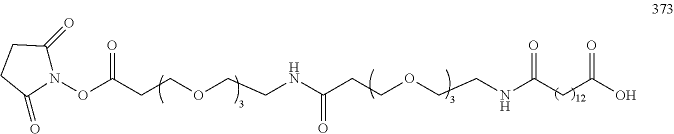

To a solution of compound 372 (4.90 g, 5.75 mmol) in THF (20 mL) in a hydrogenation bottle, Pd/C (10 wt %, 0.20 g) was added. The mixture was stirred under 1 atm H₂ overnight, filtered through Celite (filter aid), and the filtrated solution was concentrated to afford compound 373 (4.50 g, >100% yield). ESI MS m/z 762.44 ([M+H]⁺).

### Example 202. Synthesis of (S)-perfluorophenyl 2-((S)-2-(4-(bis(2-(2,5-dioxo-2,5-dihydro-1H-pyrrol-1-yl)ethyl)amino)-4-oxobutanamido)propanamido)propanoate (374)

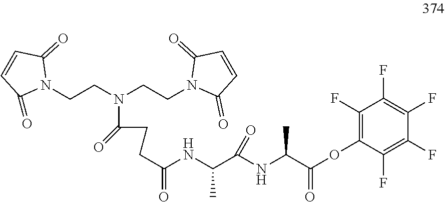 [SMILES: C[C@H](NC(=O)CCC(=O)N(CCN1C(=O)C=CC1=O)CCN1C(=O)C=CC1=O)C(=O)N[C@@H](C)C(=O)Oc1c(F)c(F)c(F)c(F)c1F]

To a solution of (S)-2-((S)-2-(4-(bis(2-(2,5-dioxo-2,5-dihydro-1H-pyrrol-1-yl)ethyl)amino)-4-oxobutanamido)propanamido)propanoic acid (47 mg, 0.084 mmol) in dichloromethane (5 mL), EDC (210 mg, 1.10 mmol) and pentafluorophenol (50.0 mg, 0.27 mmol) were added. The mixture was stirred at room temperature for 3 hours, concentrated and purified on a silica gel column (dichloromethane/EtOAc=20:1 to 5:1) to give the title compound 374 (44.6 mg, 79% yield). MS-ESI m/z: [M+H]⁺ calcd for C₂₈H₂₇F₅N₅O₉, 672.17; found, 672.17.

### Example 203. Synthesis of di-tert-butyl 1,2-bis(2-(tert-butoxy)-2-oxoethyl)hydrazine-1,2-dicarboxylate (375)

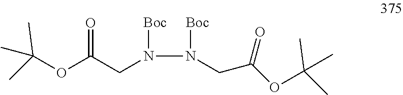 [SMILES: CC(C)(C)OC(=O)CN(C(=O)OC(C)(C)C)N(CC(=O)OC(C)(C)C)C(=O)OC(C)(C)C]

To a solution of di-tert-butyl hydrazine-1,2-dicarboxylate (8.01 g, 34.4 mmol) in DMF (150 ml), NaH (60% in oil, 2.76 g, 68.8 mmol) was added. After stirred at RT for 30 min, tert-butyl 2-bromoacetate (14.01 g, 72.1 mmol) was added. The mixture was stirred overnight, quenched with addition of methanol (3 ml), concentrated, diluted with EtOAc (100 ml) and water (100 ml), separated, and the aqueous layer was extracted with EtOAc (2×50 ml). The organic layers were combined, dried over MgSO₄, filtered, evaporated, and purified by SiO₂ column chromatography (EtOAc/Hexane 1:5 to 1:3) to afforded the title compound (12.98 g, 82% yield) as a colorless oil. MS ESI m/z calcd for C₂₂H₄₁N₂O₈ [M+H]⁺ 461.28, found 461.40.

### Example 204. Synthesis of 2,2′-(hydrazine-1,2-diyl)diacetic acid (376)

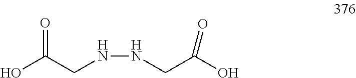

To a solution of di-tert-butyl 1,2-bis(2-(tert-butoxy)-2-oxoethyl)hydrazine-1,2-dicarboxylate (6.51 g, 14.14 mmol) in 1,4-dioxane (40 ml), HCl (12 M, 10 ml) was added. The mixture was stirred for 30 min, diluted with dioxane (20 ml) and toluene (40 ml), evaporated and co-evaporated with dioxane (20 ml) and toluene (40 ml) to dryness to afford the crude title product for the next step without further production (2.15 g, 103% yield, ˜93% pure). MS ESI m/z calcd for C₄H₉N₂O₄[M+H]⁺ 149.05, found 149.40.

### Example 205. Synthesis of 2,2′-(1,2-bis((E)-3-bromoacryloyl)hydrazine-1,2-diyl)diacetic acid (377)

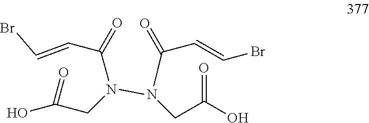 [SMILES: O=C(O)CN(C(=O)/C=C/Br)N(CC(=O)O)C(=O)/C=C/Br]

To a solution of 2,2′-(hydrazine-1,2-diyl)diacetic acid (1.10 g, 7.43 mmol) in the mixture of THF (50 ml) and NaH₂PO₄ (0.1 M, 80 ml, pH 6.0), (E)-3-bromoacryloyl bromide (5.01 g, 23.60 mmol) was added. The mixture was stirred for 6 h, concentrated and purified on SiO₂ column eluted with H₂O/CH₃CN (1:9) containing 3% formic acid to afford the title compound (2.35 g, 77% yield, ˜93% pure). MS ESI m/z calcd for C₁₀H₁₁Br₂N₂O₆ [M+H]⁺ 412.89, found 413.50.

### Example 206. Synthesis of 2,2′-(1,2-bis((E)-3-bromoacryloyl)hydrazine-1,2-diyl)diacetyl chloride (378)

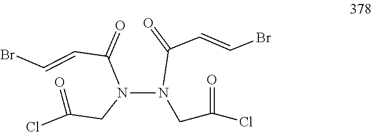 [SMILES: O=C(Cl)CN(C(=O)/C=C/Br)N(CC(=O)Cl)C(=O)/C=C/Br]

To a solution of 2,2′-(1,2-Bis((E)-3-bromoacryloyl)hydrazine-1,2-diyl)diacetic acid (210 mg, 0.509 mmol) in dichloroethane (15 ml), (COCl)₂ (505 mg, 4.01 mmol) was added, followed by addition of 0.040 ml of DMF. After stirred at RT for 2 h, the mixture was concentrated and co-evaporated with dichloroethane (2×20 ml) and toluene (2×15 ml) to dryness to afford the title crude product (which is not stable) for the next step without further purification (245 mg, 107% yield). MS ESI m/z calcd for C₁₀H₉Br₂Cl₂N₂O₄ [M+H]⁺ 448.82, 450.82, 452.82, 454.82, found 448.60, 450.60, 452.60, 454.60.

### Example 207. Synthesis of tert-butyl 2,8-dioxo-1,5-oxazocane-5-carboxylate (380)

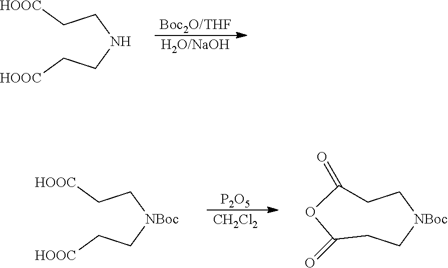

To a solution of 3,3′-azanediyldipropanoic acid (10.00 g, 62.08 mmol) in 1.0 M NaOH (300 ml) at 4° C., di-tert-butyl dicarbonate (22.10 g, 101.3 mmol) in 200 ml THF was added in 1 h. After addition, the mixture was kept to stirring for 2 h at 4° C. The mixture was carefully acidified to pH˜4 with 0.2 M H₃PO₄, concentrated in vacuo, extracted with CH₂Cl₂, dried over Na₂SO₄, evaporated and purified with flash SiO₂ chromatography eluted with AcOH/MeOH/CH₂Cl₂ (0.01:1:5) to afford 3,3′-((tert-butoxycarbonyl)azanediyl)dipropanoic acid 379 (13.62 g, 84% yield). ESI MS m/z C₁₁H₁₉NO₆ [M+H]⁺, calcd. 262.27. found 262.40.

To a solution of 3,3′-((tert-butoxycarbonyl)azanediyl)dipropanoic acid (8.0 g, 30.6 mmol) in CH₂Cl₂ (500 ml) at 0° C., phosphorus pentoxide (8.70 g, 61.30 mmol) was added. The mixture was stirred at 0° C. for 2 h and then r.t. for 1 h, filtered through short SiO₂ column, and rinsed the column with EtOAc/CH₂Cl₂ (1:6). The filtrate was concentrated and triturated with EtOAc/hexane to afford the title compound 380 (5.64 g, 74% yield). ESI MS m/z C₁₁H₁₇NO₅ [M+H]⁺, calcd. 244.11. found 244.30.

### Example 208. Synthesis of 4-(2,5-dioxo-2,5-dihydro-1H-pyrrol-1-yl)butanoic acid (381)

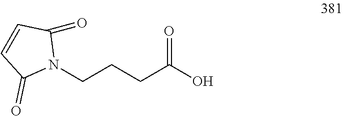 [SMILES: O=C(O)CCCN1C(=O)C=CC1=O]

To a solution of maleic anhydride (268 g, 2.73 mol) in acetic acid (1 L), 4-aminobutanoic acid (285 g, 2.76 mol) was added. After stirring at r.t. for 30 min, the reaction was refluxed for 1.5 h, cooled to r.t. and evaporated under vacuum to give a residue, which was taken up in EA, washed with water and brine, and dried over anhydrous Na₂SO₄, filtered and concentrated. The crude product was crystallized from EtOAc and PE to give a white solid (400 g, 80% yield). 1H NMR (500 MHz, CDCl3) δ 6.71 (s, 2H), 3.60 (t, J=6.7 Hz, 2H), 2.38 (t, J=7.3 Hz, 2H), 2.00-1.84 (m, 2H).

### Example 209. Synthesis of 2,5-dioxopyrrolidin-1-yl 4-(2,5-dioxo-2,5-dihydro-1H-pyrrol-1-yl)butanoate (382)

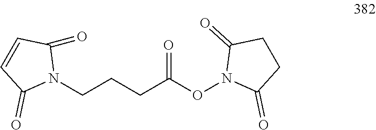 [SMILES: O=C(CCCN1C(=O)C=CC1=O)ON1C(=O)CCC1=O]

4-(2,5-dioxo-2,5-dihydro-1H-pyrrol-1-yl)butanoic acid (400 g, 2.18 mol, 1.0 eq.) was dissolved in CH₂Cl₂ (1.5 L), to which N-hydroxysuccinimide (276 g, 2.40 mmol, 1.1 eq.) and DIC (303 g, 2.40 mol, 1.1 eq.) were added at r.t. and stirred overnight. The reaction was concentrated and purified by column chromatography (1:2 petroleum ether/EtOAc) to give NHS ester as a white solid (382 g, 63% yield). ¹H NMR (500 MHz, CDCl₃) δ 6.74 (s, 2H), 3.67 (t, J=6.8 Hz, 2H), 2.85 (s, 4H), 2.68 (t, J=7.5 Hz, 2H), 2.13-2.03 (m, 2H).

### Example 210. Synthesis of (S)-2-(4-(2,5-dioxo-2,5-dihydro-1H-pyrrol-1-yl)butanamido)propanoic acid (383)

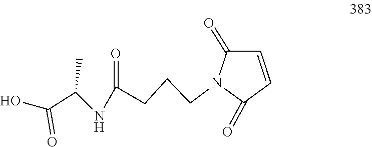 [SMILES: C[C@H](NC(=O)CCCN1C(=O)C=CC1=O)C(=O)O]

To a solution of compound 382 (7.10 g, 25.35 mmol) and alanine (3.01 g, 33.80 mmol) in DMF (50 mL) at 0° C., DIPEA (10 mL) was added. The reaction mixture was stirred at 0° C. for 0.5 h, followed by at room temperature for 1 h. Then the reaction mixture was concentrated and purified on SiO₂ column (mobile phase: DCM/MeOH=10:1 with 0.1% formic acid) to afford compound 383 (5.21 g, 81% yield). MS-ESI m/z: [M+H]⁺ calcd for C₁₁H₁₄N₂O₅, 255.09. found, 255.15.

### Example 211. Synthesis of (S)-2,5-dioxopyrrolidin-1-yl 2-(4-(2,5-dioxo-2,5-dihydro-1H-pyrrol-1-yl)butanamido)propanoate (384)

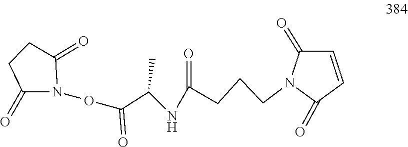 [SMILES: C[C@H](NC(=O)CCCN1C(=O)C=CC1=O)C(=O)ON1C(=O)CCC1=O]

A solution of compound 383 (5.15 g, 20.26 mmol), N-hydroxysuccinimide (2.80 g, 24.34 mmol), EDC (10.28 g, 54.10 mmol) and DIPEA (5.50 ml, 31.63 mmol) in DCM (70 ml) was stirred for 6 h, evaporated in vacuo and purified on SiO₂ column (mobile phase: DCM/EtOAc=10:1) to afford compound 384 (5.83 g, 82% yield). MS-ESI m/z: [M+H]⁺ calcd for C₁₅H₁₇N₃O₇, 351.11. found, 351.20.

### Example 212. Synthesis of (S)-1-benzyl 5-tert-butyl 2-(14-(benzyloxy)-14-oxotetradecanamido)pentanedioate (385)

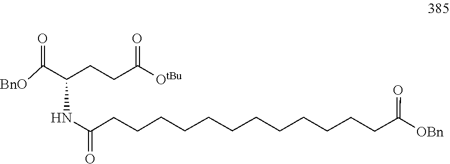 [SMILES: CC(C)(C)OC(=O)CC[C@H](NC(=O)CCCCCCCCCCCCC(=O)OCc1ccccc1)C(=O)OCc1ccccc1]

A solution of (S)-1-benzyl 5-tert-butyl 2-aminopentanedioate, HCl salt (8.70 g, 26.39 mmol), 14-(benzyloxy)-14-oxotetradecanoic acid (9.19 mmol), DIPEA (8.0 ml, 46.0 mmol) and EDC (15.3 g, 80.50 mmol) in CH₂Cl₂ (200 ml) was stirred at room temperature for 6 hour. The mixture was diluted with water (100 ml) and separated. The aqueous phase was extracted with CH₂Cl₂ (100 ml). The organic phases were combined, washed with brine, dried over Na₂SO₄, filtered, concentrated and purified on a silica gel column (dichloromethane/EtOAc=20:1 to 5:1) to give the title compound 385 (13.65 g, 83% yield). MS-ESI m/z: [M+H]⁺ calcd for C₃₇H₅₄NO₇, 624.38. found, 624.38.

### Example 213. Synthesis of (S)-5-(benzyloxy)-4-(14-(benzyloxy)-14-oxotetradecanamido)-5-oxopentanoic acid (386)

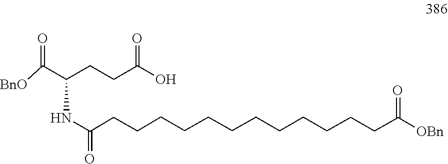 [SMILES: O=C(O)CC[C@H](NC(=O)CCCCCCCCCCCCC(=O)OCc1ccccc1)C(=O)OCc1ccccc1]

Compound 385 (12.50 g, 20.05 mmol) was dissolved in dioxane (30 mL) at 4° C., and treated with hydrochloric acid (10 mL, 36% conc) for 0.5 hours. The reaction mixture was diluted with toluene (20 ml) and DMF (20 ml), evaporated at 15° C. to give the title compound 386 (11.26 g, 99% yield). MS-ESI m/z: [M+H]⁺ calcd for C₃₃H₄₆NO₇, 568.32. found, 568.34.

### Example 214. Synthesis of (S)-35,49-dibenzyl 1-tert-butyl 16,32,37-trioxo-3,6,9,12,19,22,25,28-octaoxa-15,31,36-triazanonatetracontane-1,35,49-tricarboxylate (387)

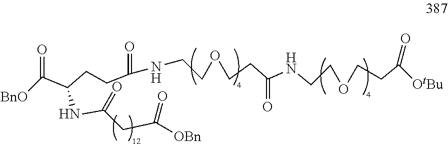

A mixture of compound 386 (10.70 g, 18.86 mmol), tert-butyl 1-amino-15-oxo-3,6,9,12,19,22,25,28-octaoxa-16-azahentriacontan-31-oate HCl salt (11.45 g, 18.93 mmol), EDC (9.51 g, 50.01 mmol) and DIPEA (4.00 ml, 23.00 mol) in CH₂Cl₂ (200 ml) was stirred overnight, diluted with brine (100 ml) and separated. The aqueous phase was extracted with CH₂Cl₂ (100 ml). The organic phases were combined, washed with brine, dried over Na₂SO₄, filtered, concentrated and purified on a silica gel column (dichloromethane/EtOAc=10:1 to 4:1) to give the title compound 387 (18.15 g, 86% yield). MS-ESI m/z: [M+H]⁺ calcd for C₅₉H₉₆N₃O₁₇, 1118.67. found, 1118.80.

### Example 215. Synthesis of(S)-18-((benzyloxy)carbonyl)-3,16,21,37-tetraoxo-1-phenyl-2,25,28,31,34,41,44,47,50-nonaoxa-17,22,38-triazatripentacontan-53-oic acid (388)

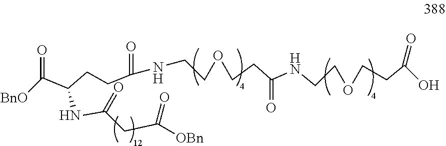

Compound 387 (10.50 g, 9.39 mmol) was dissolved in dioxane (45 mL) at 4° C., and treated with hydrochloric acid (15 mL, 36% conc) for 0.5 hours. The reaction mixture was diluted with toluene (20 ml) and DMF (20 ml), evaporated at 15° C. and purified on a silica gel column (dichloromethane/MeOH=10:1 to 6:1) to give the title compound 388 (8.67 g, 87% yield). MS-ESI m/z: [M+H]⁺ calcd for C₅₅H₈₈N₃O₁₇, 1062.60. found, 1062.68.

### Example 216. Synthesis of (18S,59S)-18-((benzyloxy)carbonyl)-59-((tert-butoxycarbonyl)amino)-3,16,21,37,53-pentaoxo-1-phenyl-2,25,28,31,34,41,44,47,50-nonaoxa-17,22,38,54-tetraazahexacontan-60-oic acid (389)

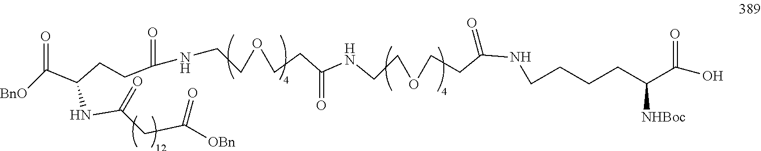

A solution of compound 388 (8.50 g, 8.01 mmol), N-hydroxysuccinimide (3.20 g, 27.82 mmol), EDC (10.28 g, 54.10 mmol) and DIPEA (6.00 ml, 34.51 mmol) in THF (150 ml) was stirred for 6 h and evaporated in vacuo to get a crude N-succinimidyl ester of (S)-18-((benzyloxy)carbonyl)-3,16,21,37-tetraoxo-1-phenyl-2,25,28,31,34,41,44,47,50-nonaoxa-17,22,38-triazatripentacontan-53-oic acid for use in next step without purification.

To a solution of (S)-6-amino-2-((tert-butoxycarbonyl)amino)hexanoic acid, HCl salt (2.75 g, 9.73 mmol) in DMF (100 mL) and 1.0 M Na₂PO₄ (pH 7.5, 55 mL), the above prepared N-succinimidyl ester was added in four portion in 1 h. The reaction mixture was stirred at room temperature for another 3 hours. After concentration, the residue was purified on a silica gel column (dichloromethane/MeOH=10:1 to 4:1) to give the title compound 389 (8.16 g, 79% yield). MS-ESI m/z: [M+H]⁺ calcd for C₆₆H₁₀₈N₅O₂₀, 1289.75. found, 1289.90.

### Example 217. Synthesis of (18S,59S)-59-amino-18-((benzyloxy)carbonyl)-3,16,21,37,53-pentaoxo-1-phenyl-2,25,28,31,34,41,44,47,50-nonaoxa-17,22,38,54-tetraazahexacontan-60-oic acid, HCl salt (390)

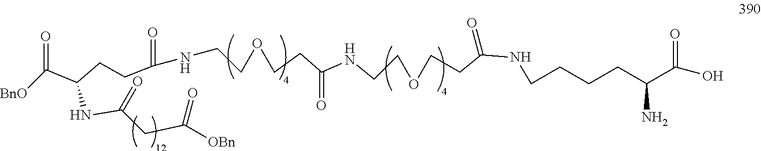

Compound 389 (8.10 g, 6.28 mmol) was dissolved in dioxane (40 mL) at 4° C., and treated with hydrochloric acid (15 mL, 36% conc) for 0.5 hours. The reaction mixture was diluted with toluene (20 ml) and DMF (20 ml), evaporated at 15° C. to give the crude title compound 390 (7.71 g, 100% yield) for next step without further purification. MS-ESI m/z: [M+H]⁺ calcd for C₆₁H₈₈N₃O₁₇, 1190.70. found, 1190.78.

### Example 218. Synthesis of (S)-2-(4-(2,5-dioxo-2,5-dihydro-1H-pyrrol-1-yl)butanamido)-propanoic acid (391)

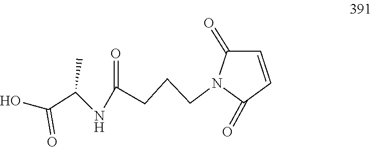 [SMILES: C[C@H](NC(=O)CCCN1C(=O)C=CC1=O)C(=O)O]

To a solution of compound 301 (7.10 g, 25.35 mmol) and alanine (3.01 g, 33.80 mmol) in DMF (50 mL) at 0° C., DIPEA (10 mL) was added. The reaction mixture was stirred at 0° C. for 0.5 h, followed by at room temperature for 1 h. Then the reaction mixture was concentrated and purified on SiO₂ column (mobile phase: DCM/MeOH=10:1 with 0.1% formic acid) to afford compound 391 (5.21 g, 81% yield). MS-ESI m/z: [M+H]⁺ calcd for C₁₁H₁₄N₂O₅, 255.09. found, 255.15.

### Example 219. Synthesis of (S)-2,5-dioxopyrrolidin-1-yl 2-(4-(2,5-dioxo-2,5-dihydro-1H-pyrrol-1-yl)butanamido)propanoate (392)

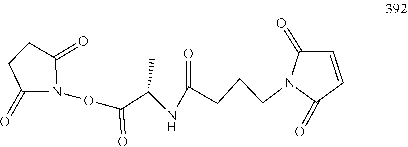 [SMILES: C[C@H](NC(=O)CCCN1C(=O)C=CC1=O)C(=O)ON1C(=O)CCC1=O]

A solution of compound 391 (5.15 g, 20.26 mmol), N-hydroxysuccinimide (2.80 g, 24.34 mmol), EDC (10.28 g, 54.10 mmol) and DIPEA (5.50 ml, 31.63 mmol) in DCM (70 ml) was stirred for 6 h, evaporated in vacuo and purified on SiO₂ column (mobile phase: DCM/EtOAc=10:1) to afford compound 392 (5.83 g, 82% yield). MS-ESI m/z: [M+H]⁺ calcd for C₁₅H₁₇N₃O₇, 351.11. found, 351.20.

### Example 220. Synthesis of (18S,59S)-18-((benzyloxy)carbonyl)-59-((S)-2-(4-(2,5-dioxo-2,5-dihydro-1H-pyrrol-1-yl)butanamido)propanamido)-3,16,21,37,53-pentaoxo-1-phenyl-2,25,28,31,34,41,44,47,50-nonaoxa-17,22,38,54-tetraazahexacontan-60-oic acid (393)

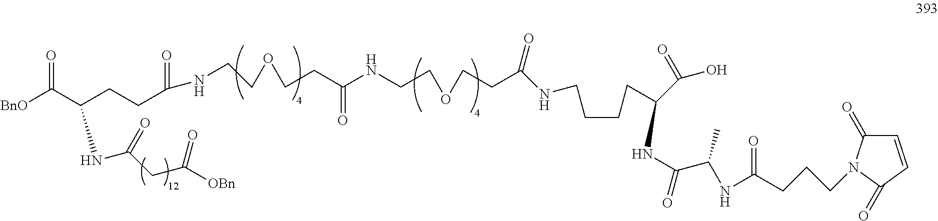

To a solution of compound 390 (7.61 g, 6.39 mmol) and compound 392 (2.90 g, 8.280 mmol) in DMF (40 mL) at 0° C., DIPEA (7 mL) was added. The reaction mixture was stirred at 0° C. for 0.5 h, followed by at room temperature for 1 h. Then the reaction mixture was concentrated and purified on SiO₂ column (mobile phase: DCM/MeOH=10:1 with 0.1% formic acid) to afford compound 393 (7.10 g, 78% yield). MS-ESI m/z: [M+H]⁺ calcd for C₇₂H₁₁₂N₇O₂₂, 1426.7782. found, 1426.7820.

### Example 221. Synthesis of (18S,59S)-18-((benzyloxy)carbonyl)-59-((S)-2-(4-(2,5-dioxo-2,5-dihydro-1H-pyrrol-1-yl)butanamido)propanamido)-3,16,21,37,53,60,63,66,69-nonaoxo-1-phenyl-2,25,28,31,34,41,44,47,50-nonaoxa-17,22,38,54,61,64,67,70-octaazadoheptacontan-72-oic acid (395)

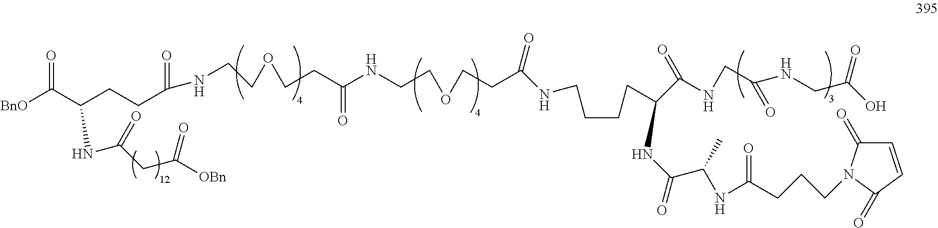

A solution of compound 393 (7.05 g, 4.94 mmol), N-hydroxysuccinimide (0.92 g, 8.00 mmol), EDC (3.01 g, 15.84 mmol) and DIPEA (1.00 ml, 5.75 mmol) in THF (50 ml) was stirred for 6 h and evaporated in vacuo to get a crude compound 394 (N-succinimidyl ester) of compound 393 for use in next step without purification.

To a solution of 2-(2-(2-aminoacetamido)acetamido)acetic acid (gly-gly-gly) HCl salt (1.67 g, 7.40 mmol) in DMF (40 mL) and 1.0 M Na₂PO₄ (pH 7.5, 15 mL), the above compound 394 was added in four portions in 1 h. The reaction mixture was stirred at room temperature for another 3 hours. After concentration, the residue was purified on a silica gel column (dichloromethane/MeOH=10:1 to 7:1) to give the title compound 395 (8.16 g, 79% yield). MS-ESI m/z: [M+H]⁺ calcd for C₇₈H₁₂₁N₁₀O₂₅, 1597.8426. found, 1597.8495.

### Example 222. Synthesis of N-(4-((18S,61S,76S)-18-((benzyloxy)carbonyl)-61-((S)-2-(4-(2,5-dioxo-2,5-dihydro-1H-pyrrol-1-yl)butanamido)propanamido)-76-methyl-3,16,21,38,55,62,65,68,71,74-decaoxo-1-phenyl-2,25,29,32,35,42,46,49,52-nonaoxa-17,22,39,56,63,66,69,72,75-nonaazaheptaheptacontanamido)benzyl)-1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium formate (396)

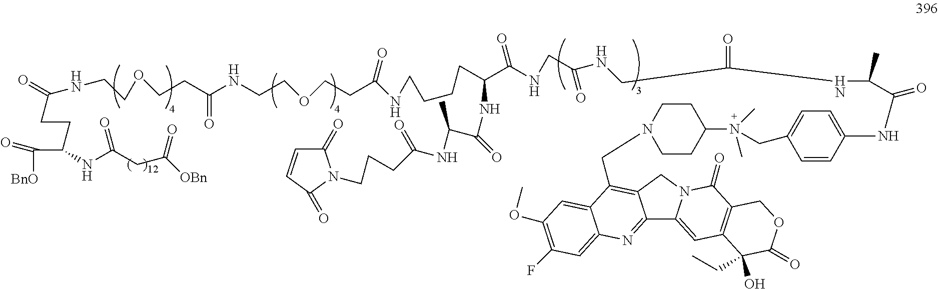

A solution of compound 395 (251 mg, 0.157 mmol), Compound 28 TFA salt (147.8 mg, 0.157 mmol), EDC (101 mg, 0.526 mmol) and DIPEA (0.10 ml, 0.575 mmol) in DMA (10 ml), was stirred at room temperature for 6 h. The mixture was evaporated in vacuo and purified by preparative C-18 HPLC (acetonitrile/water containing 0.5% formic acid, Φ=3 cm, v=20 ml/min, 90% water to 30% water in 45 min) to give compound 396 (235.8 mg, 62% yield). ESI-MS m/z: M⁺ calcd for C₁₂₁H₁₇₁FN₁₇O₃₁: 2377.2305. found 2377.2415.

### Example 223. Synthesis of N-(4-((2S,17S,60S)-60,74-dicarboxy-17-((S)-2-(4-(2,5-dioxo-2,5-dihydro-1H-pyrrol-1-yl)butanamido)propanamido)-2-methyl-4,7,10,13,16,23,40,57,62-nonaoxo-26,29,32,36,43,46,49,53-octaoxa-3,6,9,12,15,22,39,56,61-nonaazatetraheptacontan-amido)benzyl)-1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium (397)

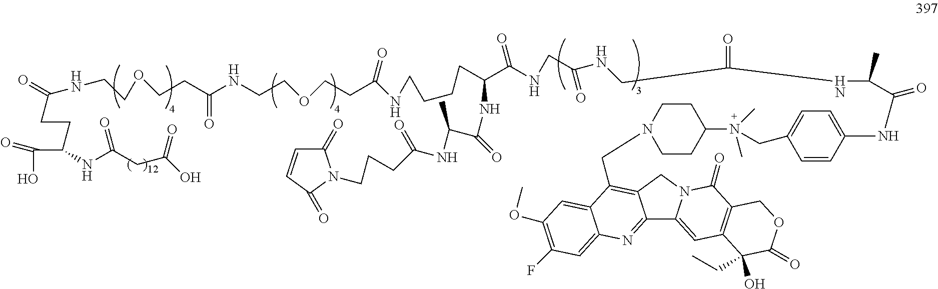

Compound 396 (110 mg, 0.0454 mmol) in DCM (2 mL) was treated with TFA (4 mL) for 1 hours. The reaction mixture was diluted with toluene (5 ml) and DMF (5 ml), evaporated, and by preparative C-18 HPLC (acetonitrile/water containing 0.5% formic acid, Φ=3 cm, v=20 ml/min, 95% water to 30% water in 45 min) to give compound 397 (70.2 mg, 69% yield). ESI-MS m/z: M⁺ calcd for C₁₀₇H₁₅₉FN₁₇O₃₁: 2197.1366. found 2197.1410.

### Example 224. Synthesis of (S)-tert-butyl (2-((2-((2-((1-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)piperidin-4-yl)amino)-2-oxoethyl)amino)-2-oxoethyl)amino)-2-oxoethyl)carbamate (398)

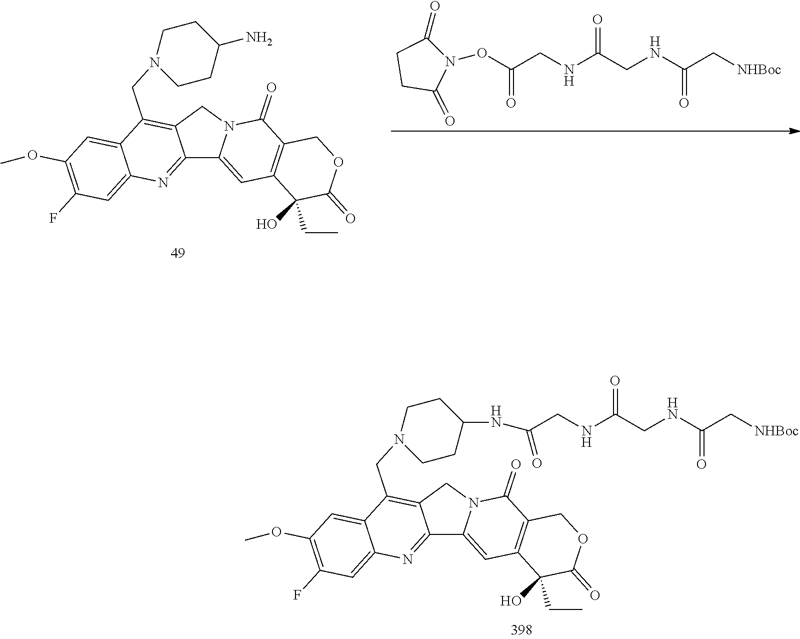 [SMILES: CC[C@@]1(O)C(=O)OCc2c1cc1n(c2=O)Cc2c-1nc1cc(F)c(OC)cc1c2CN1CCC(N)CC1 ; CC(C)(C)OC(=O)NCC(=O)NCC(=O)NCC(=O)ON1C(=O)CCC1=O ; CC[C@@]1(O)C(=O)OCc2c1cc1n(c2=O)Cc2c-1nc1cc(F)c(OC)cc1c2CN1CCC(NC(=O)CNC(=O)CNC(=O)CNC(=O)OC(C)(C)C)CC1]

In a solution of (S)-11-((4-aminopiperidin-1-yl)methyl)-4-ethyl-8-fluoro-4-hydroxy-9-methoxy-1H-pyrano[3′,4′:6,7]indolizino[1,2-b]quinoline-3,14(4H,12H)-dione, HCl salt (49) (0.805 g, 1.478 mmol) in DMF (25 ml) and 0.1 M NaH₂PO₄ pH 7.5 (50 ml), 2,5-dioxopyrrolidin-1-yl 2,2-dimethyl-4,7,10-trioxo-3-oxa-5,8,11-triazatridecan-13-oate (0.855 g, 2.214 mmol) was added in 4 portions in 3 h. After addition, the mixture was stirred for another 2 h, concentrated, extracted with EtOAc/n-butanol (1:1, 15 ml×3). The organic layers were combined, concentrated and purified on a silica gel column (dichloromethane/MeOH=12:1 to 7:1) to give the title compound 398 (0.841 g, 73% yield). MS-ESI m/z: [M+H]⁺ calcd for C₃₈H₄₇FN₇O₁₀, 780.3369; found, 780.3415.

### Example 225. Synthesis of (S)-2-amino-N-(2-((2-((1-((4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)piperidin-4-yl)amino)-2-oxoethyl)amino)-2-oxoethyl)acetamide, HCl salt

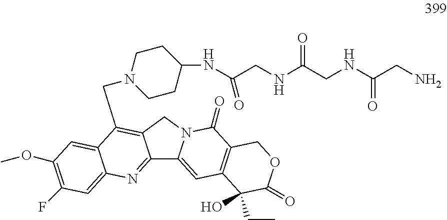 [SMILES: CC[C@@]1(O)C(=O)OCc2c1cc1n(c2=O)Cc2c-1nc1cc(F)c(OC)cc1c2CN1CCC(NC(=O)CNC(=O)CNC(=O)CN)CC1]

Compound 398 (0.810 g, 1.039 mmol) was dissolved in dioxane (25 mL) at 4° C., and treated with hydrochloric acid (10 mL, 36% conc) for 0.5 hours. The reaction mixture was diluted with toluene (15 ml) and DMF (15 ml), evaporated at 15° C. to give the crude title compound 399 (0.744 g, 100% yield) for next step without further purification. MS-ESI m/z: [M+H]⁺ calcd for C₃₃H₃₉FN₇O₈, 680.2845. found, 680.2895.

### Example 226. Synthesis of (25,10S,11S,19S)-2,19-bis((S)-18-((benzyloxy)carbonyl)-3,16,21,37,53-pentaoxo-1-phenyl-2,25,28,31,34,41,44,47,50-nonaoxa-17,22,38,54-tetraazaoctapentacontan-58-yl)-10,11-bis(4-(2,5-dioxo-2,5-dihydro-1H-pyrrol-1-yl)butanamido)-4,9,12,17-tetraoxo-3,8,13,18-tetraazaicosane-1,20-dioic acid (400)

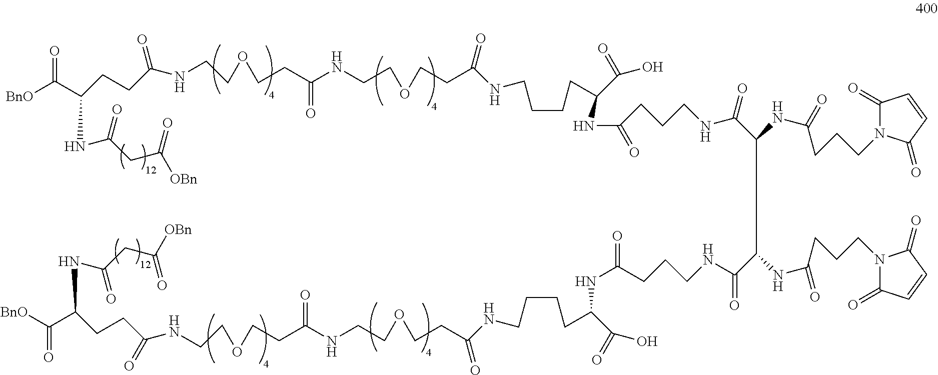

To a solution of compound 390 (2.78 g, 2.267 mmol) and compound 176 (0.951 g, 1.129 mmol) in DMF (40 mL) at 0° C., DIPEA (6 mL) was added. The reaction mixture was stirred at 0° C. for 0.5 h, followed by at room temperature for 1 h. Then the reaction mixture was concentrated and purified on SiO₂ column (mobile phase: DCM/MeOH=10:1 to 3:1 with 0.1% formic acid) to afford compound 400 (2.432 g, 72% yield). MS-ESI m/z: [M+H]⁺ calcd for C₁₅₀H₂₃₁N₁₆O₄₆, 2992.6229. found, 2992.6295.

### Example 227. Synthesis of (15S,56S,64S,65S,73S,114S)-tetrabenzyl 64,65-bis(4-(2,5-dioxo-2,5-dihydro-1H-pyrrol-1-yl)butanamido)-56,73-bis((2-((2-((2-((1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-piperidin-4-yl)amino)-2-oxoethyl)amino)-2-oxoethyl)amino)-2-oxoethyl)carbamoyl)-13,18,34,50,58,63, 66,71,79,95,111,116-dodecaoxo-22,25,28,31,38,41,44,47,82,85,88,91,98,101,104,107-hexadecaoxa-14,19,35,51,57,62,67,72,78,94,110,115-dodecaazaoctacosahectane-1,15,114,128-tetracarboxylate (401)

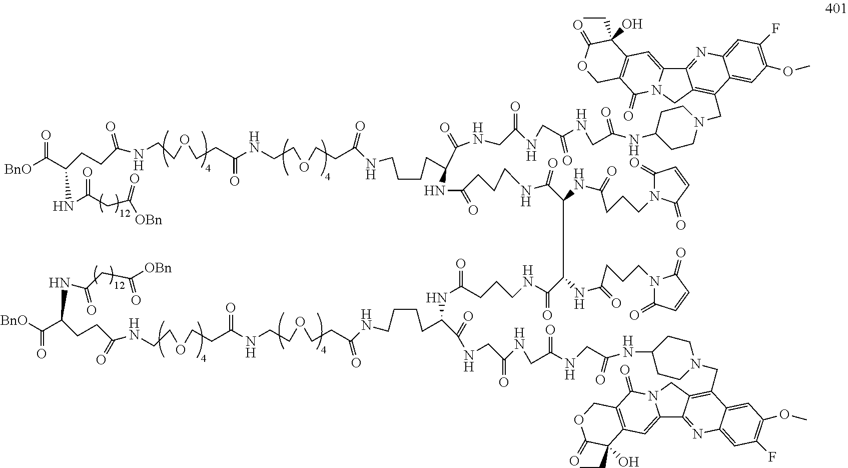

A solution of compound 399 (0.150 g, 0.209 mmol), compound 400 (0.312 g, 0.104 mmol), EDC (0.252 g, 1.311 mmol) in DMF (8 ml) was stirred for 8 h, evaporated in vacuo and purified on a silica gel column (dichloromethane/MeOH=10:1 to 7:1) to give the title compound 401 (0.301 g, 67% yield). MS-ESI m/z: [M+H]⁺ calcd for C₂₁₆H₃₀₃F₂N₃₀O₆₀, 4315.1550. found, 4315.1685.

### Example 228. Synthesis of (15S,56S,64S,65S,73S,114S)-64,65-bis(4-(2,5-dioxo-2,5-dihydro-1H-pyrrol-1-yl)butanamido)-56,73-bis((2-((2-((2-((1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)piperidin-4-yl)amino)-2-oxoethyl)amino)-2-oxoethyl)amino)-2-oxoethyl)carbamoyl)-13,18,34,50,58,63,66,71,79,95,111,116-dodecaoxo-22,25,28,31,38,41,44,47,82,85,88,91,98,101, 104,107-hexadecaoxa-14,19,35,51,57,62,67,72,78,94,110,115-dodecaazaoctacosahectane-1,15,114,128-tetracarboxylic acid (402)

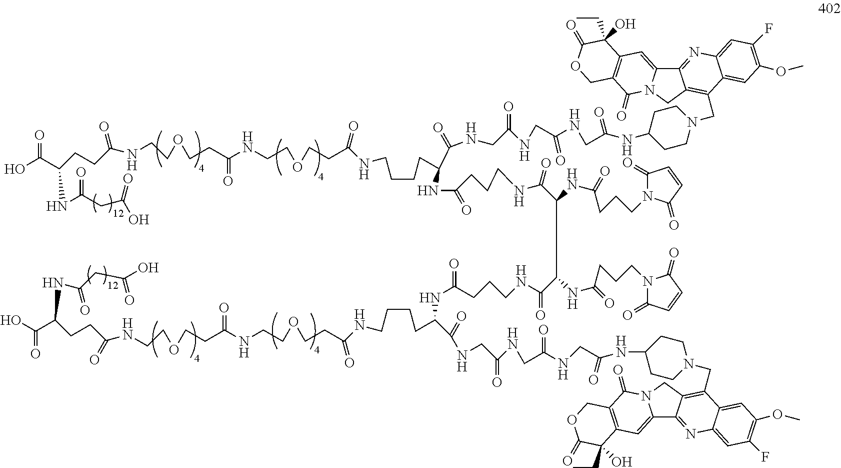

Compound 401 (105 mg, 0.0243 mmol) in DCM (2 mL) was treated with TFA (4 mL) for 1 hours. The reaction mixture was diluted with toluene (5 ml) and DMF (5 ml), evaporated, and purified by preparative C-18 HPLC (acetonitrile/water containing 0.5% formic acid, Φ=3 cm, v=20 ml/min, 95% water to 30% water in 45 min) to give compound 402 (65.3 mg, 68% yield). ESI-MS m/z: [M+H]⁺ calcd for C₁₈₈H₂₇₉F₂N₃₀O₆₀: 3954.9672. found 3954.9785.

### Example 229. Synthesis of (S)-1,1′-(((((2S,20S)-11-(tert-butoxycarbonyl)-2,20-dimethyl-4,7,15,18-tetraoxo-3,8,11,14,19-pentaazahenicosane-1,21-dioyl)bis(azanediyl))bis(4,1-phenylene))bis(methylene))bis(4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium) formate (405)

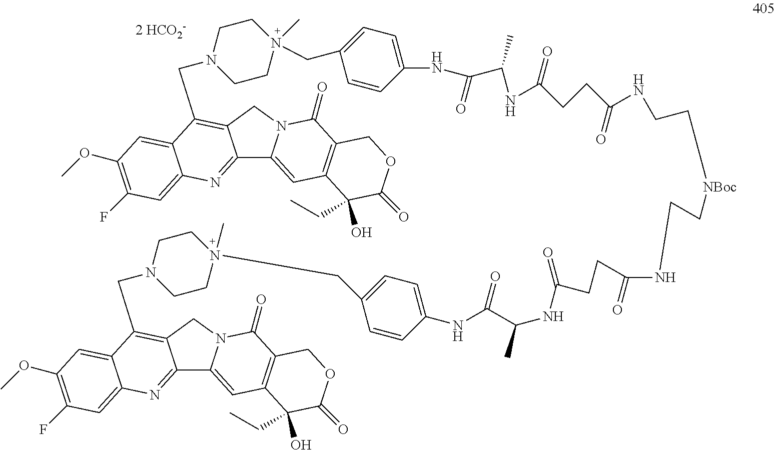

A solution of compound 40 (96 mg, 0.132 mmol) and compound 53 (26 mg, 0.066 mmol) in DMF (3 mL) was cooled to 0° C., to which HATU (50 mg, 0.132 mmol) and N, N-diisopropylethylamine (46 μL, 0.264 mmol) were added. The mixture was stirred at 0° C. for 30 min and purified by preparative C-18 HPLC (acetonitrile/water containing formic acid) to yield compound 405 (80 mg, 67%). ESI-MS m/z: [M]²⁺ calcd for C₉₁H₁₀₉F₂N₁₅O₁₈: 868.90. found 868.92.

### Example 230. Synthesis of (S)-1,1′-(((((2S,20S)-2,20-dimethyl-4,7,15,18-tetraoxo-3,8,11,14,19-pentaazahenicosane-1,21-dioyl)bis(azanediyl))bis(4,1-phenylene))bis(methylene))-bis(4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium) formate (406)

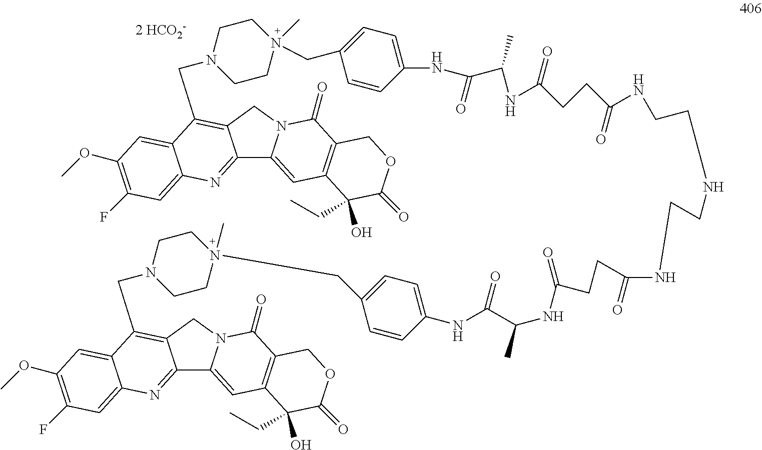

Compound 405 (80.1 mg, 0.043 mmol) was dissolved in TFA/DCM (1 mL/3 mL) and stirred at r.t. for 30 min. The reaction mixture was concentrated to dryness, yielding compound 406 (74.55 mg, 101% yield). ESI-MS m/z: [M]²⁺ calcd for C₈₆H₁₀₁F₂N₁₅O₁₆: 818.8754. found 818.8810.

### Example 231. Synthesis of (S)-1,1′-(((((2S,20S)-11-((S)-30-(4-(2,5-dioxo-2,5-dihydro-1H-pyrrol-1-yl)butanamido)-27,31-dioxo-2,5,8,11,14,17,20,23-octaoxa-26,32-diazahexatriacontan-36-oyl)-2,20-dimethyl-4,7,15,18-tetraoxo-3,8,11,14,19-pentaazahenicosane-1,21-dioyl)bis(azane-diyl))bis(4,1-phenylene))bis(methylene))bis(4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium) formate (407)

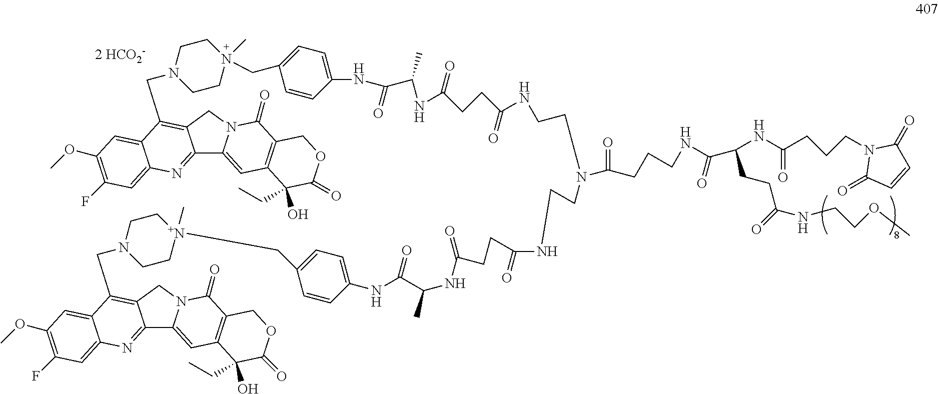

Compound 406 (74.0 mg, 0.043 mmol) and compound 7 (39 mg, 0.0516 mmol) were dissolved in DMF (3 mL), cooled to about 0° C., and then N, N-diisopropylethylamine (42 μL, 0.24 mmol) was added. The reaction was warmed to r.t. and stirred for 2 hours, concentrated, and purified by preparative C-18 HPLC (acetonitrile/water containing 2% formic acid) to give compound 407 (42 mg, 45% yield). ESI-MS m/z: M²⁺ calcd for C₁₂₀H₁₅₇F₂N₁₉O₃₀: 1191.06. found 1191.07.

### Example 232. Synthesis of 2,2′-((tert-butoxycarbonyl)azanediyl)diacetic acid (408)

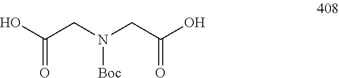 [SMILES: CC(C)(C)OC(=O)N(CC(=O)O)CC(=O)O]

Iminodiacetic acid (5.0 g, 37.6 mmol) was dissolved in THF (50 mL) and water (50 mL), mixed with NaHCO₃ (12.6 g, 150 mmol). Boc₂O (9.8 g, 45.1 mmol) was added slowly at about 5° C., then the reaction was warmed to r.t. and stirred for 2 days. The reaction mixture was diluted with water (100 mL), washed with ethyl acetate (2×30 mL), and then adjusted to pH 1.0 using concentrated HCl. The solution was extracted with ethyl acetate (3×50 mL) and the combined organic phase was washed with water (50 mL), dried over anhydrous Na₂SO₄, filtered and concentrated, triturated with ethyl acetate/petroleum ether to give a white solid (5.5 g, 63% yield). ESI-MS m/z: [M+H]⁺ calcd for C₉H₁₅NO₆: 234.09. found 234.09.

### Example 233. Synthesis of (S)-1,1′-(((((2S,2'S)-2,2′-((2,2′-((tert-butoxycarbonyl)azanediyl)bis(acetyl))bis(azanediyl))bis(propanoyl))bis(azanediyl))bis(4,1-phenylene))bis(methylene))bis(4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium) formate (409)

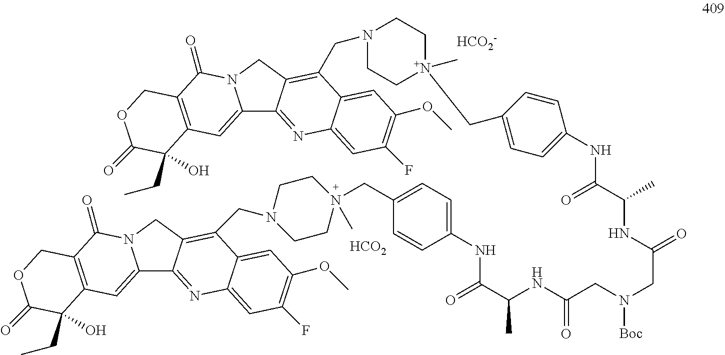

To a solution of compound 40 (109 mg, 0.12 mmol) and compound 408 (14 mg, 0.06 mmol) in DMF (3 mL), cooled to 0° C., were added HATU (50 mg, 0.132 mmol) and N, N-diisopropylethylamine (84 μL, 0.48 mmol). The reaction was stirred at 0° C. for 30 min, and then purified by preparative C-18 HPLC (acetonitrile/water containing formic acid) to give compound 409 (61 mg, 62% yield). ESI-MS m/z: [M]²⁺ calcd for C₈₃H₉₅F₂N₁₃O₁₆: 783.85. found 783.85.

### Example 234. Synthesis of (S)-1,1′-(((((2S,2'S)-2,2′-((2,2′-azanediylbis(acetyl))bis(azanediyl))bis(propanoyl))bis(azanediyl))bis(4,1-phenylene))bis(methylene))bis(4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium) formate (410)

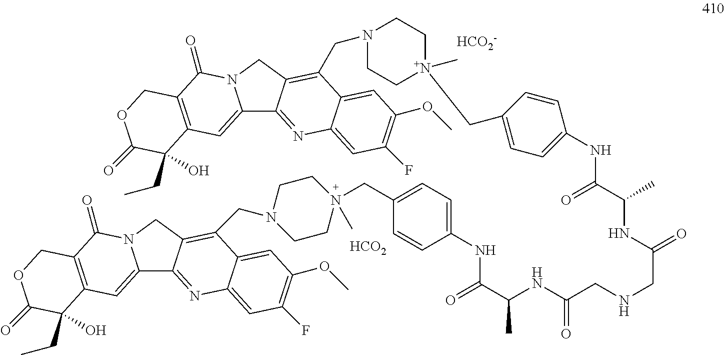

Compound 409 (61 mg, 0.036 mmol) was dissolved in TFA/DCM (1 mL/3 mL) and stirred at r.t. for 30 min. The reaction mixture was diluted with toluene (4 ml) and concentrated to dryness, yielding compound 410 (59.3 mg, >100% yield). ESI-MS m/z: [M]²⁺ calcd for C₇₈H₈₇F₂N₃O₁₄: 733.82. found 733.82.

### Example 235. Synthesis of 1-(4-((30S,41S)-30-(4-(2,5-dioxo-2,5-dihydro-1H-pyrrol-1-yl)butanamido)-37-(2-(((S)-1-((4-((4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methyl-piperazin-1-ium-1-yl)methyl)phenyl)amino)-1-oxopropan-2-yl)amino)-2-oxoethyl)-41-methyl-27,31,36,39-tetraoxo-2,5,8,11,14,17,20,23-octaoxa-26,32,37,40-tetraazadotetracontanamido)-benzyl)-4-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-1-methylpiperazin-1-ium formate (411)

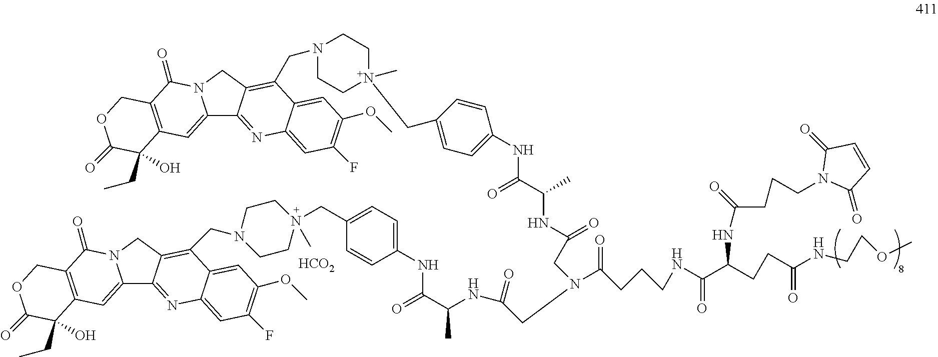

To a solution of compound 410 (65 mg, 0.036 mmol) and compound 6 (27 mg, 0.036 mmol) in DMF (3 mL), cooled to 0° C., were added HATU (17.5 mg, 0.046 mmol) and N, N-diisopropylethylamine (26 μL, 0.144 mmol). The reaction was stirred at 0° C. for 30 min, and then purified by preparative C-18 HPLC (acetonitrile/water containing 2% formic acid) to give compound 411 (39 mg, 62% yield). ESI-MS m/z: [M]²⁺ calcd for C₁₁₂H₁₄₃F₂N₁₇O₂₈: 1106.01; found 1106.01.

### Example 236. Synthesis of (S)-N,N′-(((((2S,2'S)-2,2′-((2,2′-((tert-butoxycarbonyl)azanediyl)bis(acetyl))bis(azanediyl))bis(propanoyl))bis(azanediyl))bis(4,1-phenylene))bis(methylene))bis(1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium) formate (412)

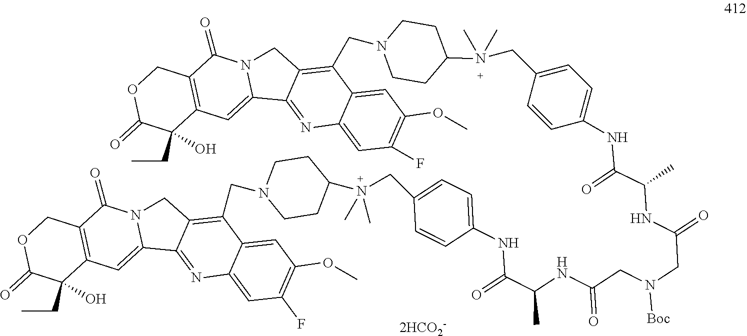

To a solution of compound 28 (106 mg, 0.113 mmol) and compound 408 (13 mg, 0.056 mmol) in DMF (3 mL), cooled to 0° C., were added HATU (43 mg, 0.113 mmol) and N, N-diisopropylethylamine (39 μL, 0.226 mmol). The reaction was stirred for 4 h, and then purified by preparative C-18 HPLC (acetonitrile/water containing formic acid) to give compound 412 (71 mg, 74% yield). ESI-MS m/z: [M]²⁺ calcd for C₈₇H₁₀₃F₂N₁₃O₁₆: 811.8801. found 811.8875.

### Example 237. Synthesis of (S)-N,N′-(((((2S,2'S)-2,2′-((2,2′-azanediylbis(acetyl))bis-(azanediyl))bis(propanoyl))bis(azanediyl))bis(4,1-phenylene))bis(methylene))bis(1-(((S)-4-ethyl-8-fluoro-4-hydroxy-9-methoxy-3,14-dioxo-3,4,12,14-tetrahydro-1H-pyrano[3′,4′:6,7]indolizino-[1,2-b]quinolin-11-yl)methyl)-N,N-dimethylpiperidin-4-aminium) formate (413)

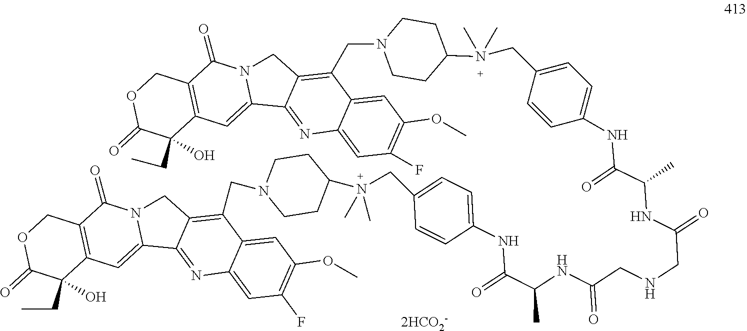

Compound 412 (71 mg, 0.041 mmol) was dissolved in TFA/DCM (1 mL/3 mL) and stirred at r.t. for 30 min. The reaction mixture was diluted with toluene (5 ml) and concentrated to dryness, yielding compound 413 (70 mg, >100 yield). ESI-MS m/z: [M]²⁺ calcd for C₈₂H₉₅F₂N₁₃O₄: 761.8539. found 761.8595.

### Example 238. Synthesis of N-(4-((30S,41S)-30-(4-(2,5-dioxo-2,5-dihydro-1H-pyrrol-1-yl)butanamido)-37-(2-(((S)-1-((4-(((1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)piperidin-4-yl)dimethylammonio)methyl)phenyl)amino)-1-oxopropan-2-yl)amino)-2-oxoethyl)-41-methyl-27,31,36,39-tetraoxo-2,5,8,11,14,17,20,23-octaoxa-26,32,37,40-tetraazadotetracontanamido)-benzyl)-1-(((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)-N,N-dimethylpiperidin-4-aminium formate (414)

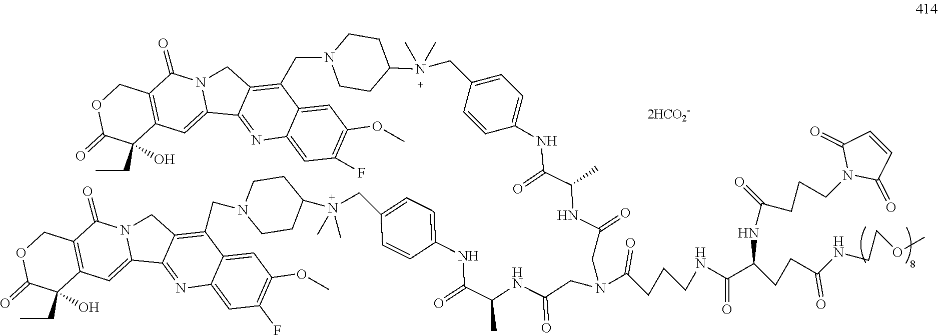

To a solution of compound 413 (70 mg, ˜0.041 mmol) and compound 6 (32 mg, 0.041 mmol) in DMF (4 mL), cooled to 0° C., were added HATU (19 mg, 0.049 mmol) and N, N-diisopropylethylamine (28 μL, 0.164 mmol). The reaction was stirred for 4 h, and then purified by preparative C-18 HPLC (acetonitrile/water containing formic acid) to give compound 414 (43 mg, 45% yield). ESI-MS m/z: [M]²⁺ calcd for C₁₁₆H₁₅₁F₂N₁₇O₂₈: 1134.04. found 1134.04.

### Example 239. Synthesis of 4-((S)-2-((tert-butoxycarbonyl)amino)propanamido) benzyl (((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)carbamate (415)

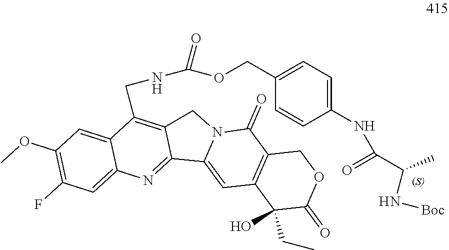

To a solution of compound 15 (83 mg, 0.282 mmol) in DCM (2 mL) were added triphosgene (30 mg, 0.094 mmol) and triethylamine (37 μL, 0.282 mmol). The reaction was then warmed to r.t. and stirred for 1 h, concentrated to dryness. Compound 30 (100 mg, 0.235 mmol) was dissolved in DMF (2 mL) and cooled to 0° C., to which triethylamine (37 μL, 0.282 mmol) and the above chloroformate were added. After the addition was completed, the resulting mixture was stirred at 0° C. for 1 h and then purified by preparative C-18 HPLC (acetonitrile/water containing formic acid) to give compound 415 (122 mg, 70% yield). ESI-MS m/z: [M+H]⁺ calcd for C₃₈H₄₀FN₅O₁₀: 746.2838. found 746.2898.

### Example 240. Synthesis of 4-((S)-2-aminopropanamido)benzyl (((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)carbamate (416)

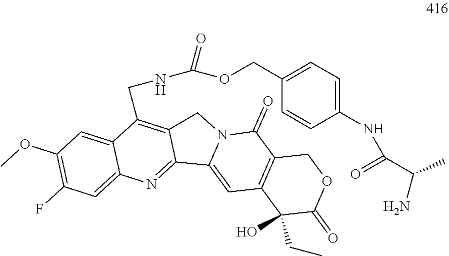 [SMILES: CC[C@@]1(O)C(=O)OCc2c1cc1n(c2=O)Cc2c-1nc1cc(F)c(OC)cc1c2CNC(=O)OCc1ccc(NC(=O)[C@H](C)N)cc1]

Compound 415 (122.5 mg, 0.164 mmol) was dissolved in TFA/DCM (1 mL/3 mL) and stirred at r.t. for 30 min. The reaction mixture was diluted with toluene (4 ml) and concentrated to dryness, yielding compound 416 (120.2 mg, 0.100% yield). ESI-MS m/z: [M+H]⁺ calcd for C₃₃H₃₂FN₅O₈: 646.22. found 646.22.

### Example 241. Synthesis of tert-butyl bis(2-(((S)-1-((4-((((((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)carbamoyl)oxy)methyl)phenyl)amino)-1-oxopropan-2-yl)amino)-2-oxoethyl)carbamate (417)

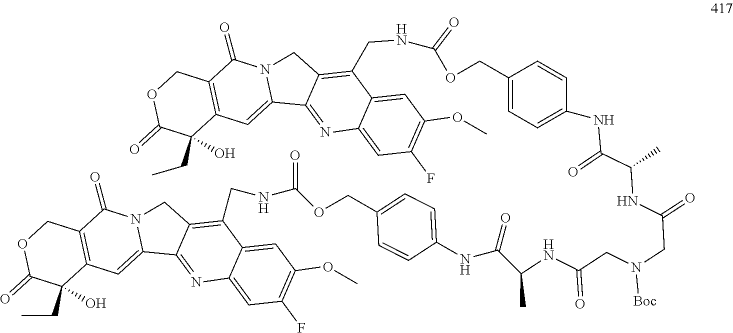 [SMILES: CC[C@@]1(O)C(=O)OCc2c1cc1n(c2=O)Cc2c-1nc1cc(F)c(OC)cc1c2CNC(=O)OCc1ccc(NC(=O)[C@H](C)NC(=O)CN(CC(=O)N[C@@H](C)C(=O)Nc2ccc(COC(=O)NCc3c4c(nc5cc(F)c(OC)cc35)-c3cc5c(c(=O)n3C4)COC(=O)[C@]5(O)CC)cc2)C(=O)OC(C)(C)C)cc1]

To a solution of compound 416 (120 mg, 0.164 mmol) and compound 408 (19 mg, 0.082 mmol) in DMF (3 mL), cooled to 0° C., were added HATU (62 mg, 0.164 mmol) and N, N-diisopropylethylamine (57 μL, 0.328 mmol). The reaction was stirred for 8 h, concentrated and then purified by preparative C-18 HPLC (acetonitrile/water containing formic acid) to give compound 417 (171 mg, 70% yield). ESI-MS m/z: [M+H]⁺ calcd for C₇₅H₇₆F₂N₁₁O₂₀: 1488.5237. found 1488.5295.

### Example 242. Synthesis of ((((2S,2'S)-2,2′-((2,2′-azanediylbis(acetyl))bis(azanediyl))-bis(propanoyl))bis(azanediyl))bis(4,1-phenylene))bis(methylene) bis((((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)carbamate) (418)

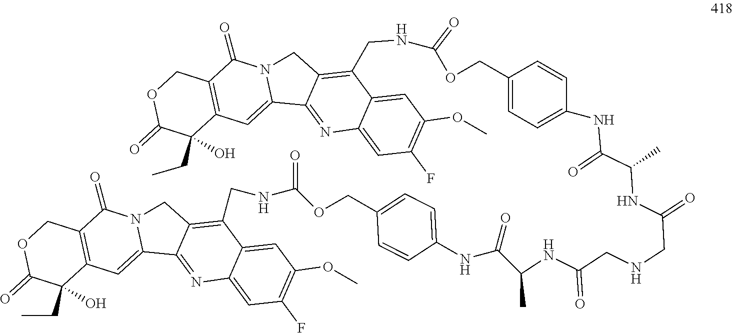 [SMILES: CC[C@@]1(O)C(=O)OCc2c1cc1n(c2=O)Cc2c-1nc1cc(F)c(OC)cc1c2CNC(=O)OCc1ccc(NC(=O)[C@H](C)NC(=O)CNCC(=O)N[C@@H](C)C(=O)Nc2ccc(COC(=O)NCc3c4c(nc5cc(F)c(OC)cc35)-c3cc5c(c(=O)n3C4)COC(=O)[C@]5(O)CC)cc2)cc1]

Compound 417 (171 mg, 0.115 mmol) was dissolved in TFA/DCM (1 mL/3 mL) and stirred at r.t. for 30 min. The reaction mixture was concentrated to dryness, yielding compound 418 (172 mg, >100% yield). ESI-MS m/z: [M+H]⁺ calcd for C₇₀H₆₈F₂N₁₁O₁₈: 1388.46. found 1388.46.

### Example 243. Synthesis of ((((2S,2'S)-2,2′-(((S)-30-(4-(2,5-dioxo-2,5-dihydro-1H-pyrrol-1-yl)butanamido)-27,31,36-trioxo-37-(2-oxoethyl)-2,5,8,11,14,17,20,23-octaoxa-26,32,37-triazanonatriacontan-39-oyl)bis(azanediyl))bis(propanoyl))bis(azanediyl)) bis(4,1-phenylene))bis(methylene) bis((((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)carbamate) (2017)

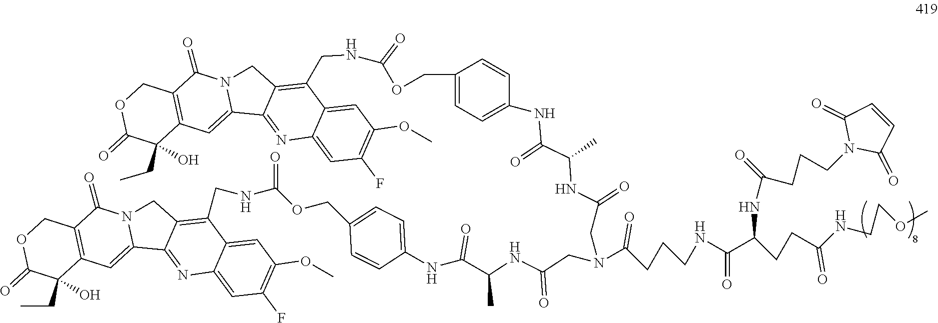

To a solution of compound 418 (172 mg, 0.115 mmol) and compound 6 (87 mg, 0.115 mmol) in DMF (3 mL), cooled to 0° C., were added HATU (52 mg, 0.138 mmol) and N, N-diisopropylethylamine (40 μL, 0.23 mmol). The reaction was stirred for 4 h, and then purified by preparative C-18 HPLC (acetonitrile/water containing formic acid) to give compound 419 (122 mg, 50% yield). ESI-MS m/z: [M+H]⁺ calcd for C₁₀₄H₁₂₃F₂N₁₅O₃₂: 2132.84. found 2132.84.

### Example 244. Synthesis of 2-amino-4-fluoro-5-hydroxybenzaldehyde (420)

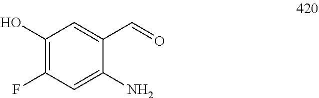 [SMILES: Nc1cc(F)c(O)cc1C=O]

To a solution of 4-fluoro-3-methxybenzaldehyde (770 mg, 5.0 mmol) in concentrated sulfuric acid (10 mL) at 0° C. was added fuming nitric acid (95%, 315 mg, 4.8 mmol) dropwise. The mixture was stirred at r.t for 1 h, then poured into ice water, and filtered. The filter cake was washed with water and then dried. The resulting residue was dissolved in DMF (20 mL), lithium chloride (1.6 g, 25 mmol) was added and the mixture was refluxed for 4h then poured into water, and concentrated hydrochloric acid was added dropwise to reach pH 4. The solution was extracted with ethyl acetate and the organic layer was washed with brine, dried and concentrated in vacuo. To the resulting residue were added ethanol/water (25 mL, 4:1), iron powder (1.21 g, 22 mmol) and ammonium chloride (433 mg, 8.1 mmol). The mixture was stirred at 80° C. for 2h, and solid was then filtered off. Water was added to the filtrate, and the resulting mixture was extracted with ethyl acetate. The organic layer was washed with brine, dried, and concentrated, purified by column chromatography to give the title compound (125 mg, 16 yield). ESI-MS m/z: [M+H]⁺ calcd for C₇H₆FNO₂ 156.04. found 156.04.

### Example 245. Synthesis of (S)-4-ethyl-8-fluoro-4,9-dihydroxy-TH-pyrano[3′,4′:6,7]

indolizino[1,2-b]quinoline-3,14(4H,12H)-dione (421)

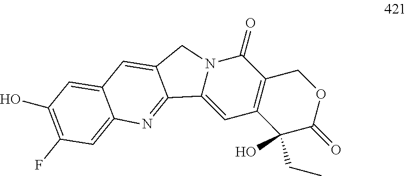 [SMILES: CC[C@@]1(O)C(=O)OCc2c1cc1n(c2=O)Cc2cc3cc(O)c(F)cc3nc2-1]

Compound 420 (0.125 g, 0.805 mmol) and compound 25 (0.202 g, 0.76 mmol) were dissolved in anhydrous toluene (40 mL), and p-toluenesulfonic acid (13 mg, 0.076 mmol) was added. The suspension was heated at reflux for 2 days and allowed to cool to r.t. After removal of about two-thirds of toluene, the residue was filtered and the filter cake was washed with dichloromethane, air-dried to give compound 421 (0.26 g, 90% yield) as a gray powdery solid. ESI-MS m/z: [M+H]⁺ calcd for C₂₀H₁₆FN₂O₅: 383.10. found 383.10.

### Example 246. Synthesis of (S)-tert-butyl (2-(9-ethyl-5-fluoro-9-hydroxy-10,13-dioxo-9,10-dihydro-[1,3]oxazino[5,6-f]pyrano[3′,4′:6,7]indolizino[1,2-b]quinolin-2(1H,3H,12H,13H,15H)-yl)ethyl)carbamate (422)

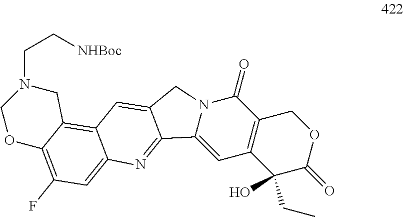 [SMILES: CC[C@@]1(O)C(=O)OCc2c1cc1n(c2=O)Cc2cc3c4c(c(F)cc3nc2-1)OCN(CCNC(=O)OC(C)(C)C)C4]

A solution of N-Boc-ethylenediamine (50 mg, 0.31 mmol) and paraformaldehyde (70 mg, 0.78 mmol) in 1,4-dioxane (5 mL) was heated at about 100° C. for 2 h, then cooled to r.t. and compound 421 (100 mg, 0.26 mmol) was added. The reaction was heated to 100° C. again and stirred for 2 days, cooled to r.t. and purified by preparative C-18 HPLC (acetonitrile/water containing formic acid) to give compound 422 (117 mg, 80% yield). ESI-MS m/z: [M+H]⁺ calcd for C₂₉H₃₁FN407: 567.22. found 567.22.

### Example 247. Synthesis of (S)-2-(2-aminoethyl)-9-ethyl-5-fluoro-9-hydroxy-2,3,12,15-tetrahydro-[1,3]oxazino[5,6-f]pyrano[3′,4′:6,7]indolizino[1,2-b]quinoline-10,13(1H,9H)-dione (2023)

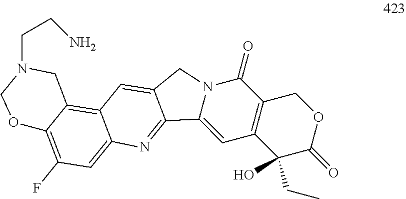 [SMILES: CC[C@@]1(O)C(=O)OCc2c1cc1n(c2=O)Cc2cc3c4c(c(F)cc3nc2-1)OCN(CCN)C4]

Compound 422 (117 mg, 0.208 mmol) was dissolved in TFA/DCM (2 mL/6 mL) and stirred at r.t. for 1 h. The reaction mixture was concentrated to dryness, yielding a yellow solid 423 (117 g, >100 yield). ESI-MS m/z: [M+H]⁺ calcd for C₂₄H₂₃FN₄O₅: 467.17. found 467.17.

### Example 248. Synthesis of (S)-2-(4-(2,5-dioxo-2,5-dihydro-1H-pyrrol-1-yl)butanamido)-N1-(4-((2-((S)-9-ethyl-5-fluoro-9-hydroxy-10,13-dioxo-9,10-dihydro-[1,3]oxazino[5,6-f]pyrano[3′,4′:6,7]indolizino[1,2-b]quinolin-2(1H,3H,12H,13H,15H)-yl)ethyl)amino)-4-oxobutyl)-N5-(2,5,8,11,14,17,20,23-octaoxapentacosan-25-yl)pentanediamide (424)

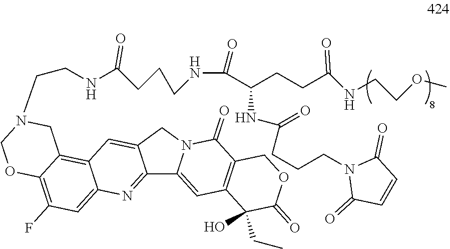

To a solution of compound 423 (120 mg, 0.208 mmol) and compound 7 (193 mg, 0.208 mmol) in DMF (5 mL), cooled to 0° C., was added N, N-diisopropylethylamine (72 μL, 0.416 mmol). The reaction was warmed to r.t. and stirred for 2 h, concentrated and purified by preparative HPLC (acetonitrile/water containing formic acid) to give compound 424 (100 mg, 40% yield). ESI-MS m/z: [M+H]⁺ calcd for C₅₈H₇₉FN₈O₁₉: 1211.54. found 1211.54.

### Example 249. Synthesis of (S)-9-ethyl-5-fluoro-9-hydroxy-2-(2-hydroxyethyl)-2,3,12,15-tetrahydro-[1,3]oxazino[5,6-f]pyrano[3′,4′:6,7]indolizino[1,2-b]quinoline-10,13(1H,9H)-dione (425)

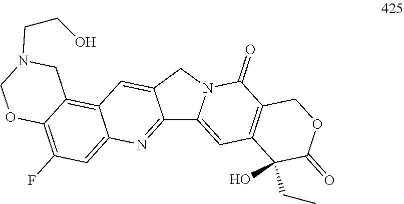

A solution of ethanolamine (19 mg, 0.31 mmol) and paraformaldehyde (70 mg, 0.78 mmol) in 1,4-dioxane (5 mL) was heated at about 100° C. for 2 h, then cooled to r.t. and compound 421 (100 mg, 0.26 mmol) was added. The reaction was heated to 100° C. again and stirred for 2 days, cooled to r.t. and purified by preparative HPLC (acetonitrile/water containing formic acid) to give compound 425 (91 mg, 75% yield). ESI-MS m/z: [M+H]⁺ calcd for C₂₄H₂₂FN₃O₆: 468.15. found 468.15.

### Example 250. Synthesis of (S)-N1-(4-((2-aminoethyl)amino)-4-oxobutyl)-2-(4-(2,5-dioxo-2,5-dihydro-1H-pyrrol-1-yl)butanamido)-N5-(2,5,8,11,14,17,20,23-octaoxapentacosan-25-yl)pentanediamide (426)

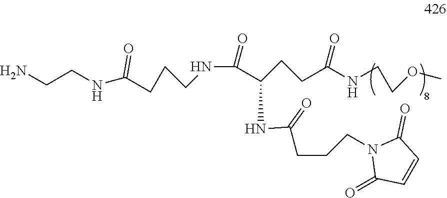

A solution of 1,2-diethyl-diamine (300 mg, 4.99 mmol) in THF (15 mL) and 1.0 M NaH₂PO₄ (15 ml) was adjusted to pH 7.5 with 0.1 M H₃PO₄. The mixture was cooled to 4˜10° C., and the compound 7 (700 mg, 0.75 mmol) was added in four portions in 1 h. After additionally stirred for 2 h, the mixture was concentrated and purified by preparative HPLC (acetonitrile/water containing 1% formic acid) to give compound 426 (528 mg, 82% yield). ESI-MS m/z: [M+H]⁺ calcd for C₃₆H₆₅N₆O₁₄: 805.4560. found 805.4595.

### Example 251. Synthesis of 2-((S)-9-ethyl-5-fluoro-9-hydroxy-10,13-dioxo-9,10-dihydro-[1,3]oxazino[5,6-f]pyrano[3′,4′:6,7]indolizino[1,2-b]quinolin-2(1H,3H,12H,13H,15H)-yl)ethyl ((S)-30-(4-(2,5-dioxo-2,5-dihydro-1H-pyrrol-1-yl)butanamido)-27,31,36-trioxo-2,5,8,11,14,17,20,23-octaoxa-26,32,37-triazanonatriacontan-39-yl)carbamate (428)

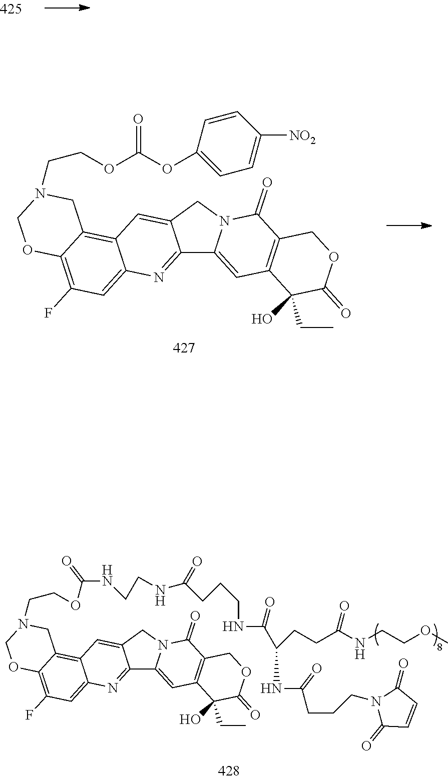

To a solution of compound 425 (30 mg, 0.0642 mmol) in dry THF (5 mL) and DIPEA (15 μl, 0.091 mmol) at 0° C., 4-nitrophenyl carbonochloridate (13 mg, 0.0646 mmol) was added. The mixture was stirred for 4 h at 0° C. to form (S)-2-(9-ethyl-5-fluoro-9-hydroxy-10,13-dioxo-9,10-dihydro-[1,3]oxazino[5,6-f]pyrano[3′,4′:6,7]indolizino[1,2-b]quinolin-2(1H,3H,12H,13H,15H)-yl)ethyl (4-nitrophenyl) carbonate (427), which was used directly for the next step without isolation. Then to the mixture, compound 426 (55 mg, 0.0643 mmol) and DIPEA (10 l, 61.2 mmol) were added. The mixture was stirred for 4 h, concentrated and purified by preparative C-18 HPLC (acetonitrile/water containing 1% formic acid) to give compound 428 (39 mg, 47% yield). ESI-MS m/z: [M+H]⁺ calcd for C₆₁H₈₅FN₉O₂₁: 1298.5845. found 1298.5935.

### Example 252. Synthesis of bis(2,5-dioxopyrrolidin-1-yl) 4,4′-((((tert-butoxycarbonyl)azanediyl)bis(ethane-2,1-diyl))bis(azanediyl))bis(4-oxobutanoate) (431)

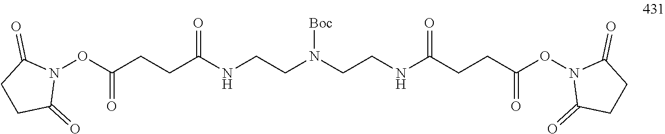 [SMILES: CC(C)(C)OC(=O)N(CCNC(=O)CCC(=O)ON1C(=O)CCC1=O)CCNC(=O)CCC(=O)ON1C(=O)CCC1=O]

To a solution of compound 53 (201 mg, 0.5 mmol) in DCM (10 mL), were added EDC HCl (287 mg, 1.5 mmol) and NHS (173 mg, 1.5 mmol). The reaction was stirred at r.t. for 1 h and then diluted with DCM (50 mL), washed with water (2×10 mL), dried over anhydrous Na₂SO₄, filtered and concentrated to give compound 431 (297 mg, 100% yield). ESI-MS m/z: [M+H]⁺ calcd for C₂₅H₃₅N₅O₁₂: 598.22. found 598.22.

### Example 253. Synthesis of 11-(tert-butoxycarbonyl)-4,7,15,18-tetraoxo-3,8,11,14,19-pentaazahenicosane-1,21-dioic acid (432)

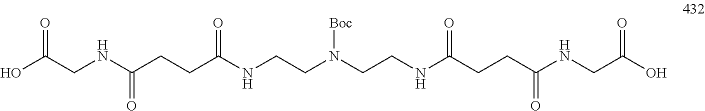 [SMILES: CC(C)(C)OC(=O)N(CCNC(=O)CCC(=O)NCC(=O)O)CCNC(=O)CCC(=O)NCC(=O)O]

H-Gly-OH (94 mg, 1.25 mmol) was dissolved in water (10 mL) and NaHCO₃ (168 mg, 2.00 mmol) was added, followed by compound 431 (297 mg, 0.5 mmol). The reaction was then stirred at r.t. for 1 h and concentrated, purified by preparative HPLC (acetonitrile/water containing formic acid) to give compound 432 (155 mg, 60% yield). ESI-MS m/z: [M+H]⁺ calcd for C₂₁H₃₅N₅O₁₀: 518.23. found 518.23.

### Example 254. Synthesis of bis(perfluorophenyl) 11-(tert-butoxycarbonyl)-4,7,15,18-tetraoxo-3,8,11,14,19-pentaazahenicosane-1,21-dioate (433)

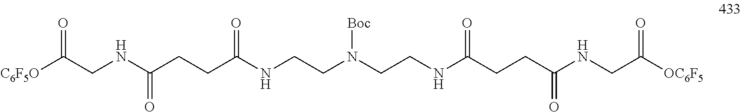

To a solution of compound 432 (110 mg, 0.12 mmol) in DCM (5 mL) were added pentafluorophenol (48 mg, 0.26 mmol) and EDC HCl (50 mg, 0.26 mmol). The reaction was stirred at r.t. for 2 h and then diluted with DCM (50 mL), washed with water (2×10 mL), dried over anhydrous Na₂SO₄, filtered and concentrated to give compound 433 (180 mg, 100% yield). ESI-MS m/z: [M+H]⁺ calcd for C₃₃H₃₃F₁₀N₅O₁₀: 850.20. found 850.20.

### Example 255. Synthesis of tert-butyl bis(2-(4-((2-((((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)amino)-2-oxoethyl)amino)-4-oxobutanamido)ethyl)carbamate (434)

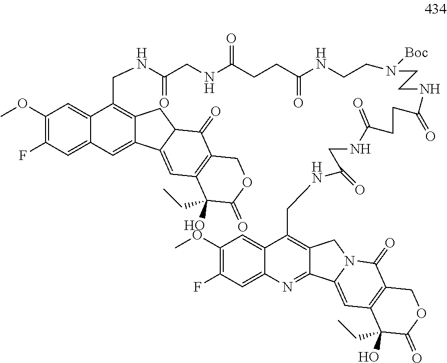 [SMILES: CC[C@@]1(O)C(=O)OCC2=C1C=C1c3cc4cc(F)c(OC)cc4c(CNC(=O)CNC(=O)CCC(=O)NCCN(CCNC(=O)CCC(=O)NCC(=O)NCc4c5c(nc6cc(F)c(OC)cc46)-c4cc6c(c(=O)n4C5)COC(=O)[C@]6(O)CC)C(=O)OC(C)(C)C)c3CC1C2=O]

To a solution of compound 30 (55 mg, 0.13 mmol) in DMF (1 mL) were added DIPEA (27 mg, 0.21 mmol) and compound 433 (50 mg, 0.06 mmol) over an ice-water bath. The reaction was warmed to r.t. and stirred for 1 h, then concentrated, purified by preparative HPLC (acetonitrile/water containing formic acid) to give compound 434 (20 mg, 25% yield). ESI-MS m/z: [M+H]⁺ calcd for C₆₅H₇₂F₂N₁₁O₁₈: 1332.49. found 1332.49.

### Example 256. Synthesis of N1,N1′-(azanediylbis(ethane-2,1-diyl))bis(N4-(2-((((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)amino)-2-oxoethyl)succinamide) (435)

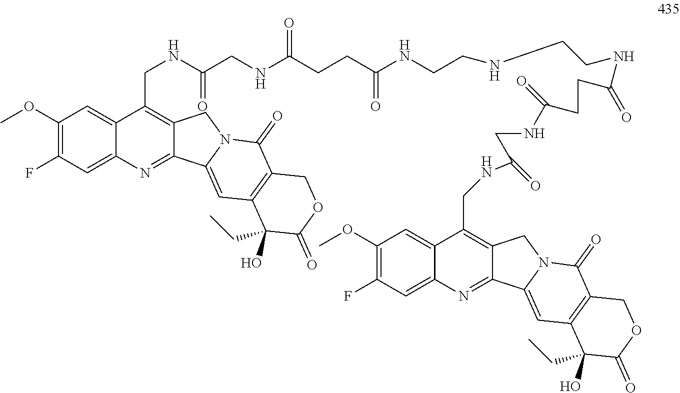 [SMILES: CC[C@@]1(O)C(=O)OCc2c1cc1n(c2=O)Cc2c-1nc1cc(F)c(OC)cc1c2CNC(=O)CNC(=O)CCC(=O)NCCNCCNC(=O)CCC(=O)NCC(=O)NCc1c2c(nc3cc(F)c(OC)cc13)-c1cc3c(c(=O)n1C2)COC(=O)[C@]3(O)CC]

Compound 434 (20 mg, 0.015 mmol) was dissolved in TFA/DCM (0.5 mL/1 mL) and stirred at r.t. for 2 h. The reaction mixture was concentrated to dryness, yielding a yellow solid (18.5 mg, 100% yield). ESI-MS m/z: [M+H]⁺ calcd for C₆₀H₆₃F₂N₁₁O₁₆: 1232.44. found 1232.44.

### Example 257. Synthesis of (S)-2-(4-(2,5-dioxo-2,5-dihydro-1H-pyrrol-1-yl)butanamido)-N1-(1-((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)-13-(2-(4-((2-((((S)-4-ethyl-8-fluoro-4-hydroxy-9-methoxy-3,14-dioxo-3,4,12,14-tetrahydro-1H-pyrano[3′,4′:6,7]indolizino[1,2-b]quinolin-11-yl)methyl)amino)-2-oxoethyl)amino)-4-oxobutanamido)ethyl)-3,6,9,14-tetraoxo-2,5,10,13-tetraazaheptadecan-17-yl)-N5-(2,5,8,11,14,17,20,23-octaoxapentacosan-25-yl)pentanediamide (436)

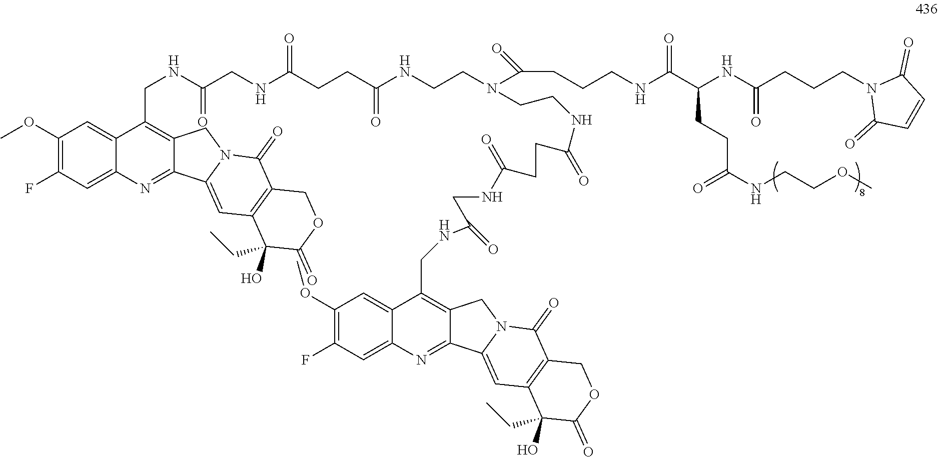

To an ice cold solution of compound 6 (11 mg, 0.015 mmol) in DMF (1 mL), were added HATU (11.4 mg, 0.03 mmol) and N, N-diisopropylethylamine (10 μL, 0.06 mmol), followed by compound 435 (18.5 mg, 0.015 mmol). The reaction was stirred at 0° C. for 1 h, and then purified by preparative HPLC (acetonitrile/water containing formic acid) to give compound 436 (10 mg, 34% yield). ESI-MS m/z: [M+H]⁺ calcd for C₉₄H₁₁₉F₂N₁₅O₃₀: 1976.82. found 1976.82. Example 258. General method of Preparation of Conjugate C1-005, C1-008, C1-021, C1-022, C1-029, C1-031, C1-035, C1-041, C1-042, C1-043, C1-047, C1-050, C1-056, C1-061, C1-064, C1-110, C1-177, C1-188, C1-200, C1-208, C1-213, C1-226, C1-238, C1-243, C1-247, C1-305, C1-306, C1-311, C1-362, C1-397, C1-402, C1-407, C1-411, C1-414, C1-419, C1-424, and C1-428.

To a shaken solution containing 2.0 mL of 10 mg/ml Her2 antibody (Herceptin) or Trop-2 antibody or EGFR antibody in pH 6.0-8.0 PBS buffer, 0.70˜2.0 mL of 100 mM NaH₂PO₄, pH 6.5-8.5 buffers and TCEP (35-70 μL, 20 mM in water), the compound 5, 8, 21, 22, 29, 31, 35, 41, 42, 43, 47, 50, 56, 61, 64, 110, 177, 188, 200, 213, 226, 238, 243, 247, 305, 306, 311, 362, 397, 402, 407, 411, 414, 419, 424, and 428 (35-90 μL, 20 mM in DMA) were added independently, followed by addition of 4-(azidomethyl)benzoic acid (30-100 μL, 20 mM in pH 7.5, PBS buffer). The mixture was incubated at RT for 4-18 h, then DHAA (100˜150 μL, 50 mM) was added in. After continuous incubation at RT overnight, the mixture was purified on G-25 column eluted with 100 mM NaH₂PO₄, 50 mM NaCl pH 6.0-7.5 buffer to afford 11.2-18.5 mg of the conjugate compounds C1-005, C1-008, C1-021, C1-022, C1-029, C1-031, C1-035, C1-041, C1-042, C1-043, C1-047, C1-050, C1-056, C1-061, C1-064, C1-110, C1-177, C1-188, C1-200, C1-208, C1-213, C1-226, C1-238, C1-243, C1-247, C1-305, C1-306, C1-311, C1-362, C1-397, C1-402, C1-407, C1-411, C1-414, C1-419, C1-424, and C1-428 (82%-95% yield) accordingly in 8.3-15.2 ml of the NaH₂PO₄ buffer. The drug/antibody ratio (DAR) was 4.1-8.0, wherein DAR was determined via UPLC-QTOF mass spectrum and by UV (the CPT compounds here used the Extinction coefficient: E_{280 nm}=4992 M⁻¹ cm⁻¹; E_{377 nm}=16730 M⁻¹ cm⁻¹). It was 95˜99% monomer analyzed by SEC HPLC (Tosoh Bioscience, Tskgel G3000SW, 7.8 mm ID×30 cm, 0.5 ml/min, 100 min).

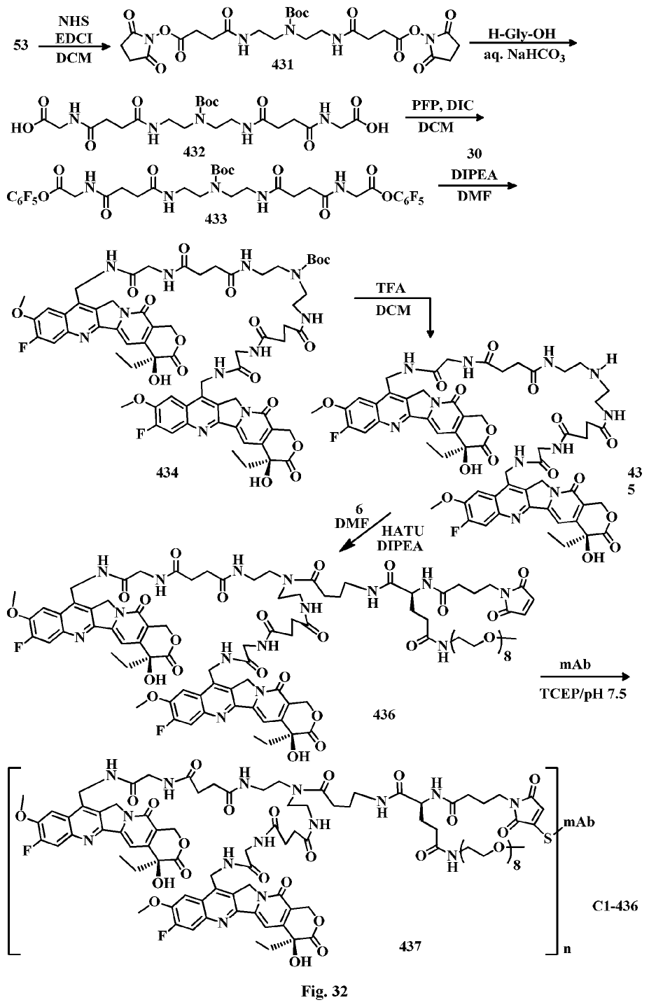

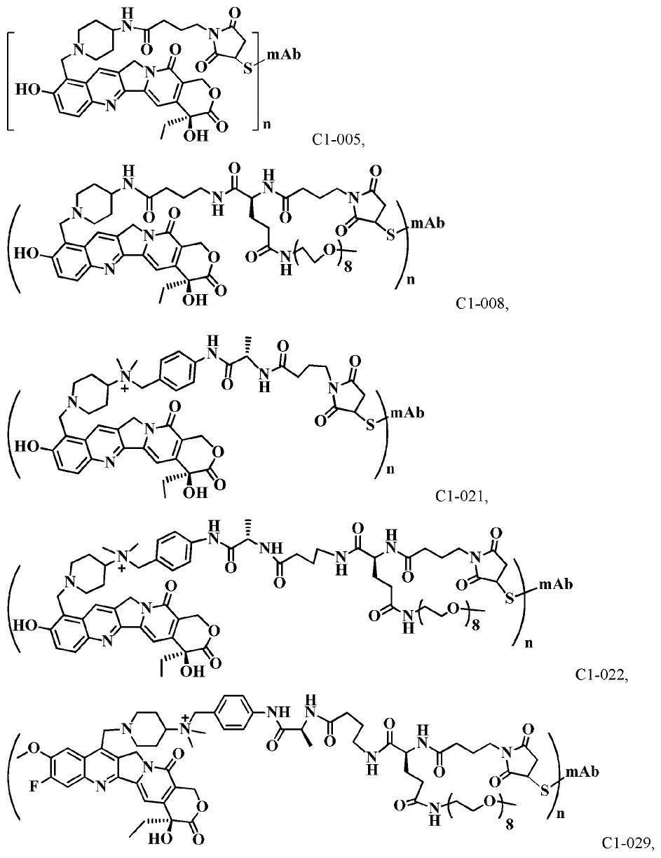

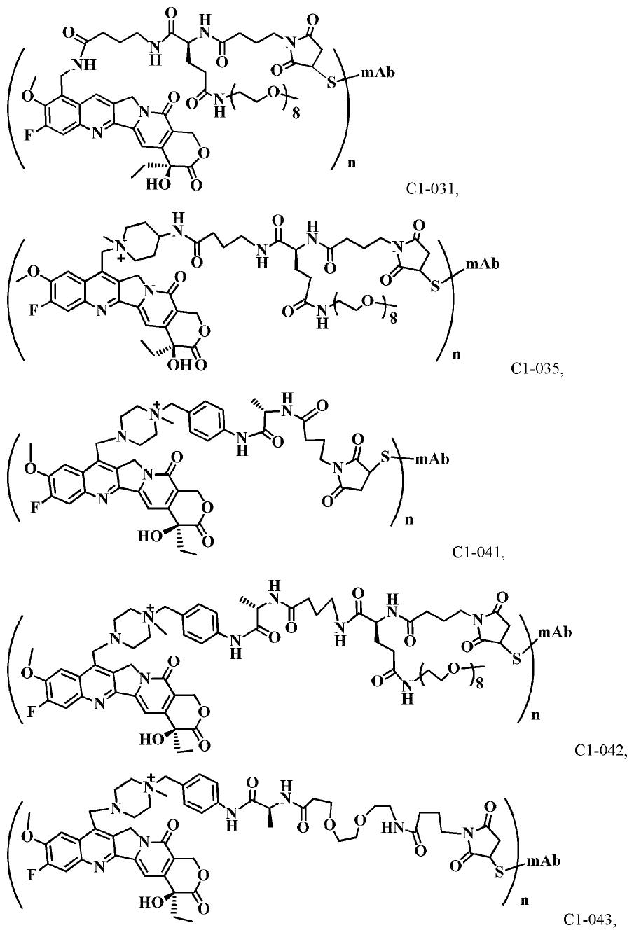

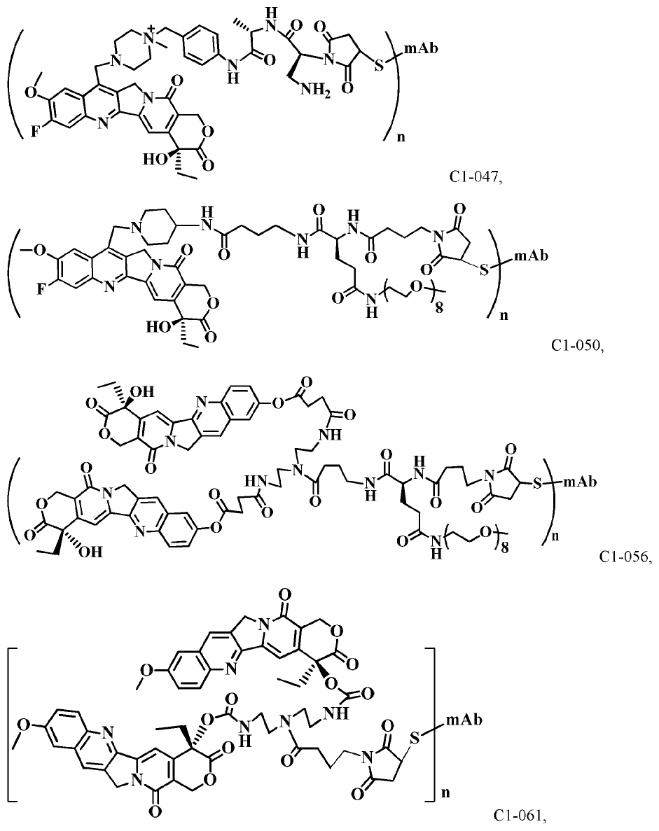

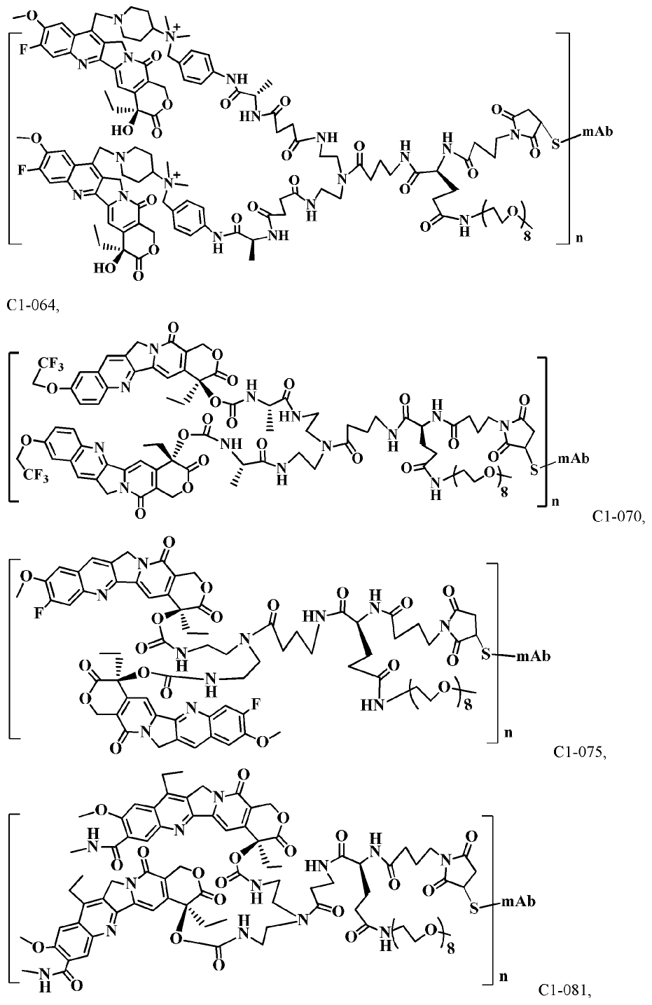

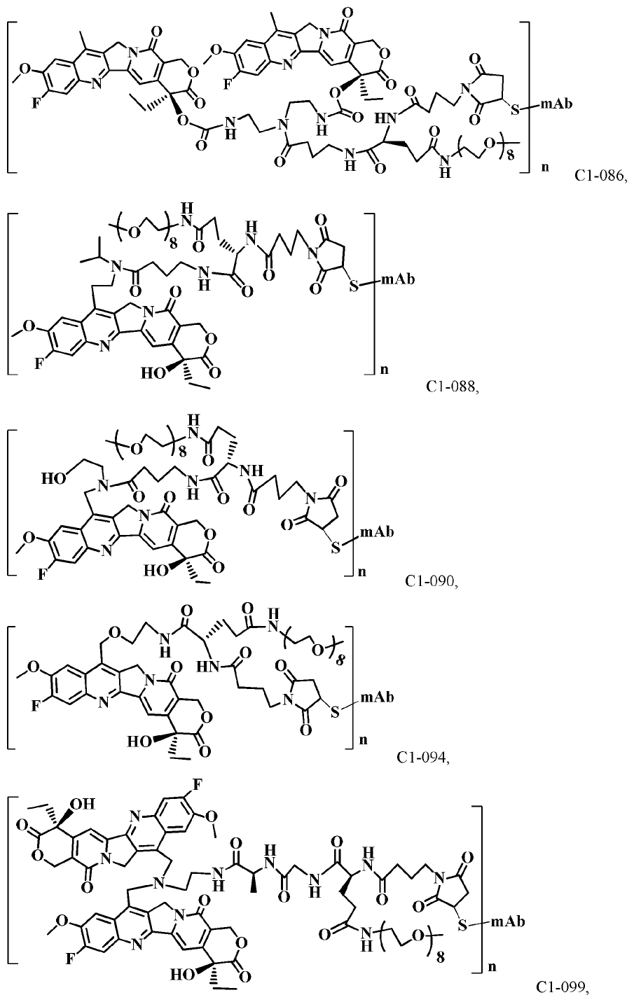

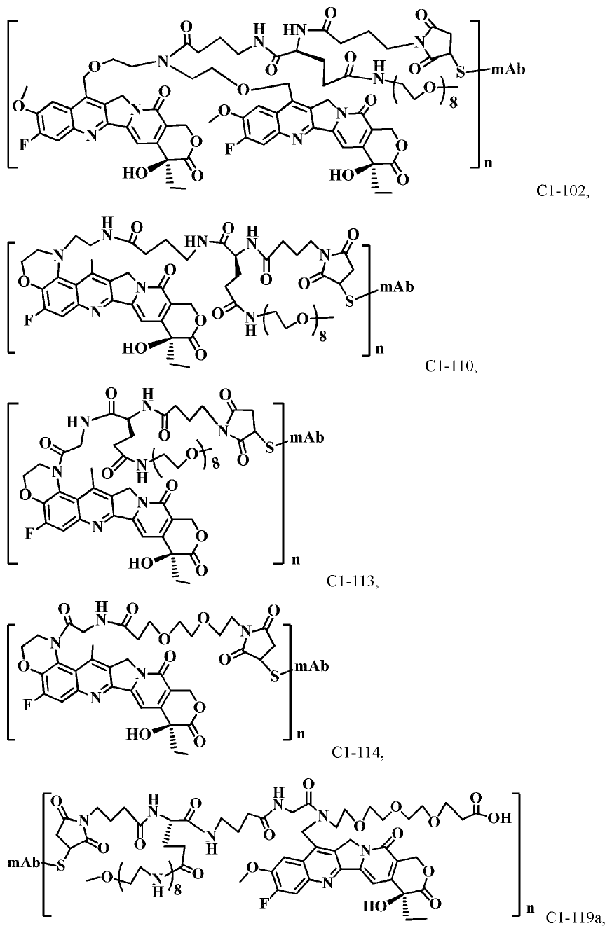

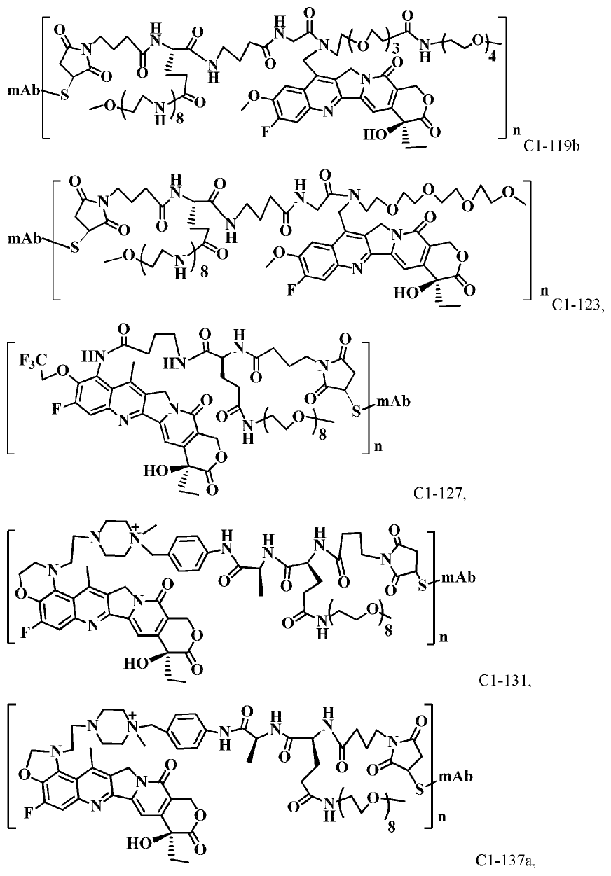

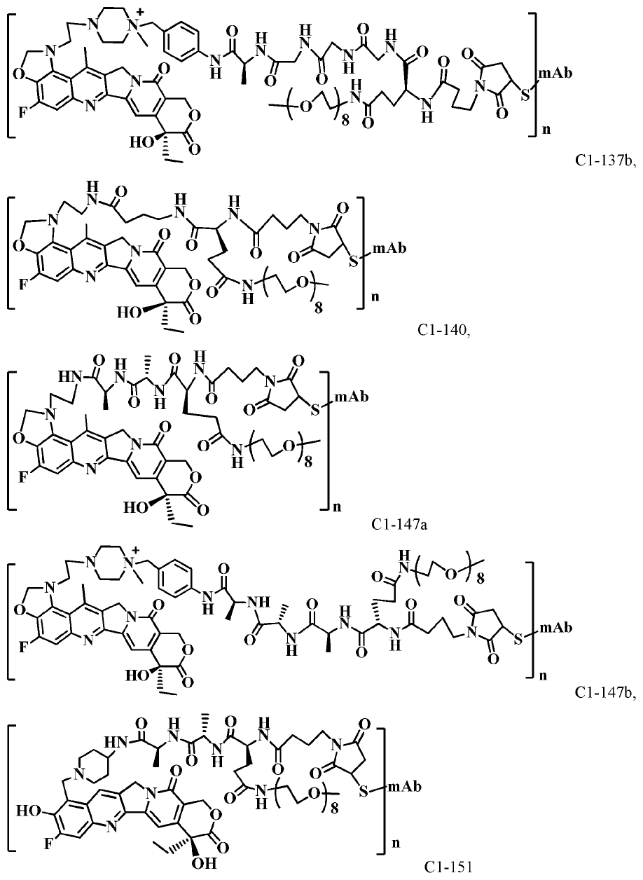

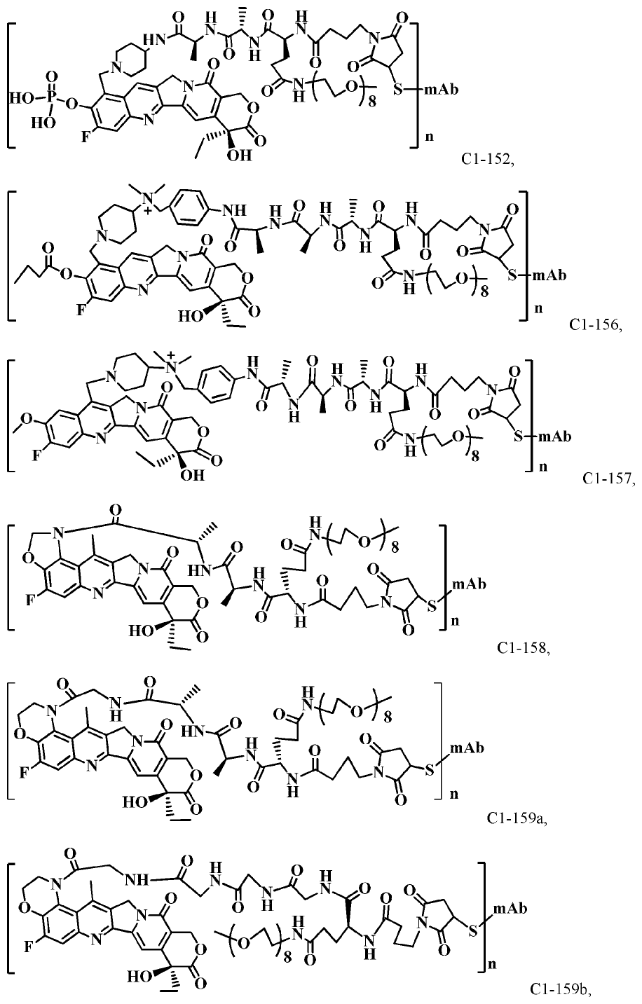

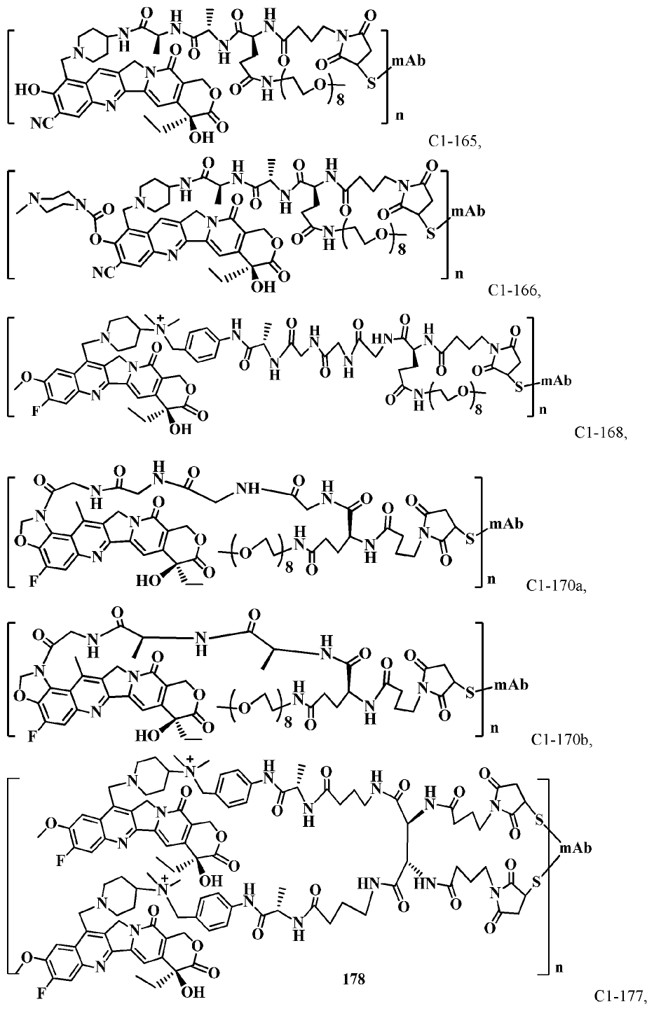

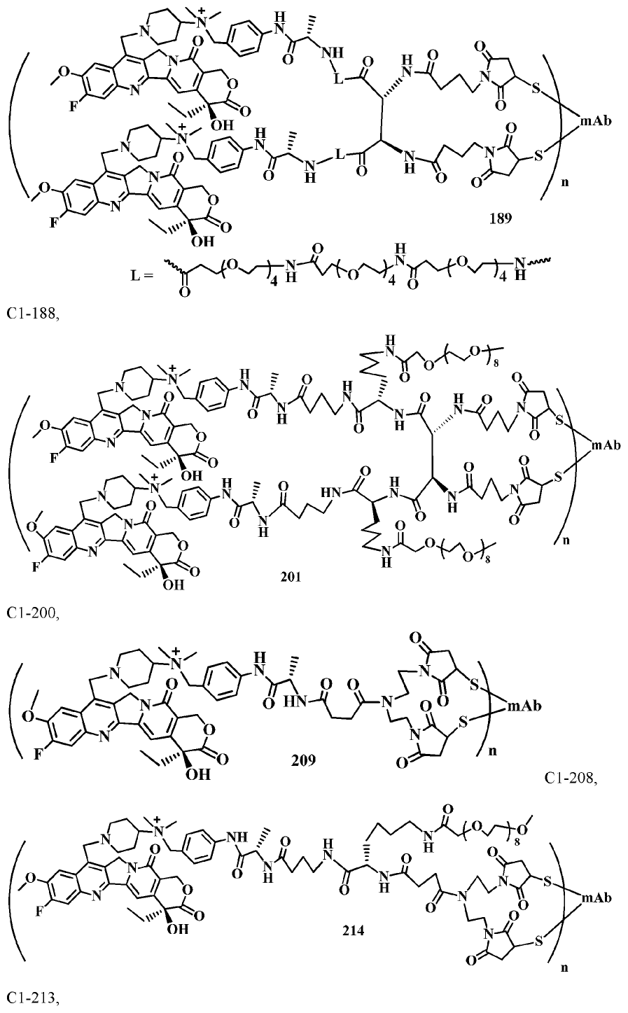

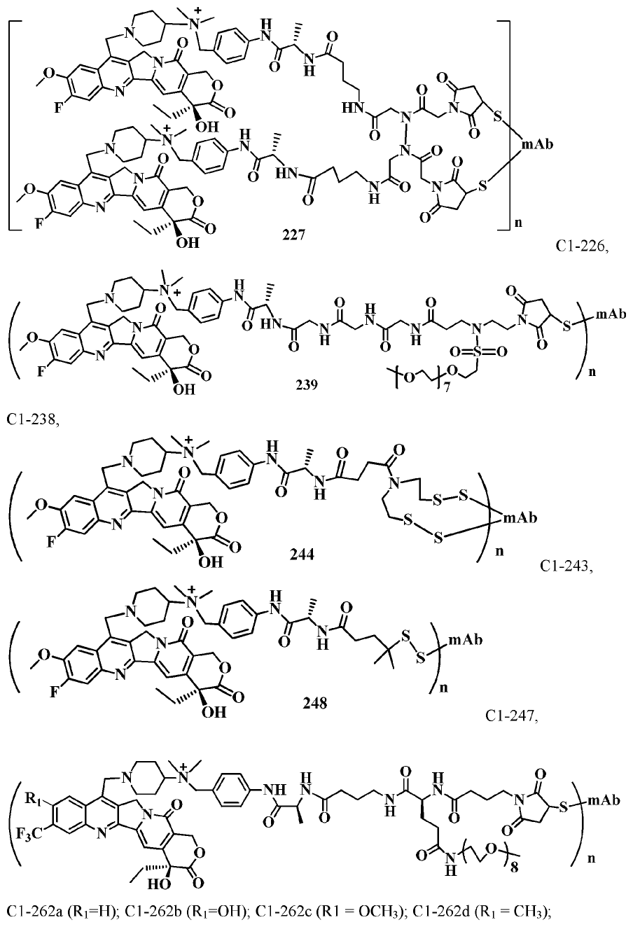

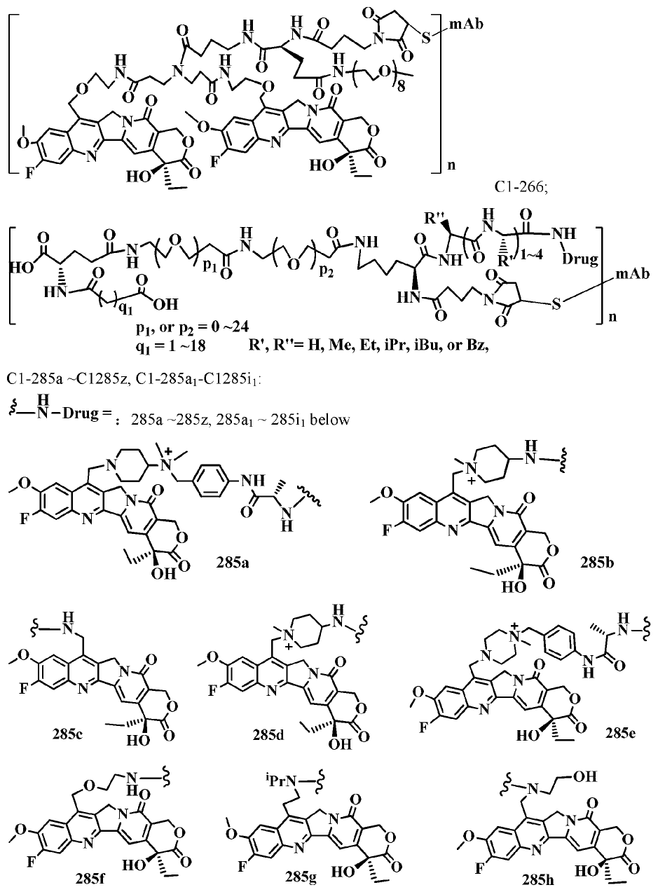

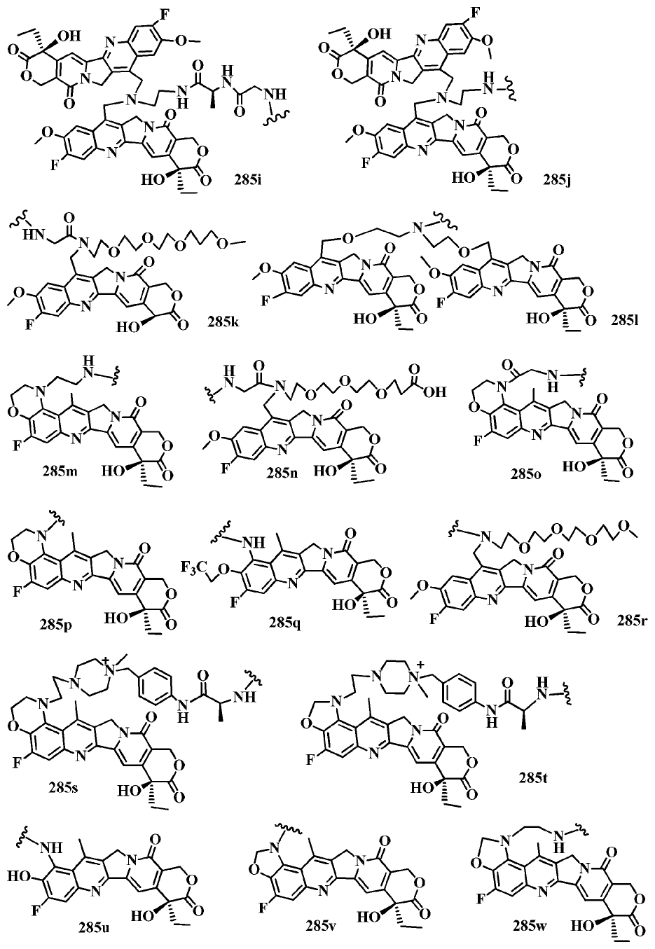

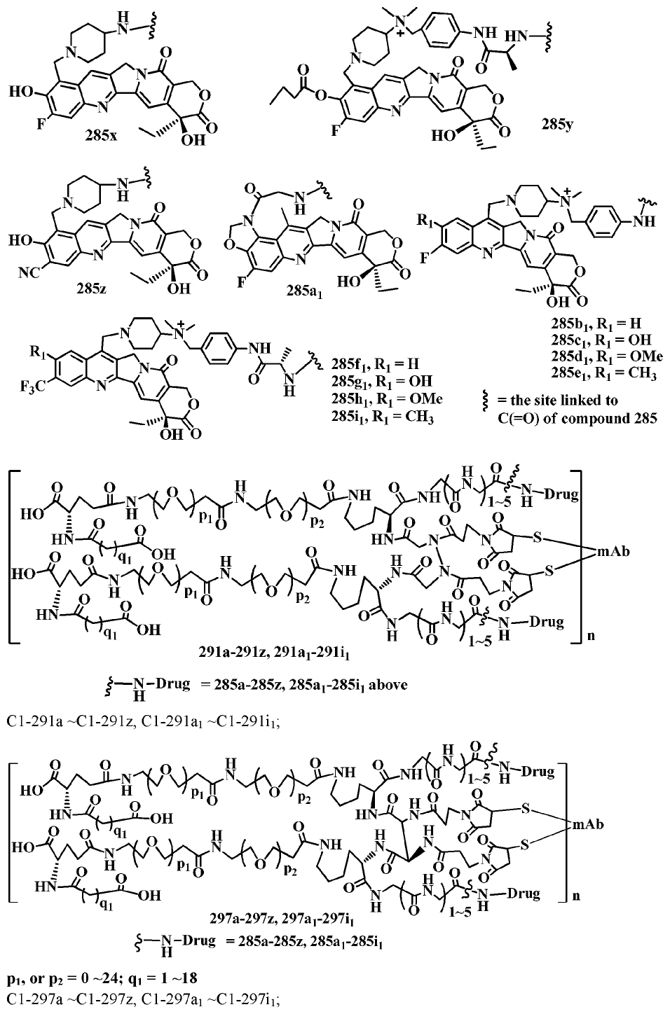

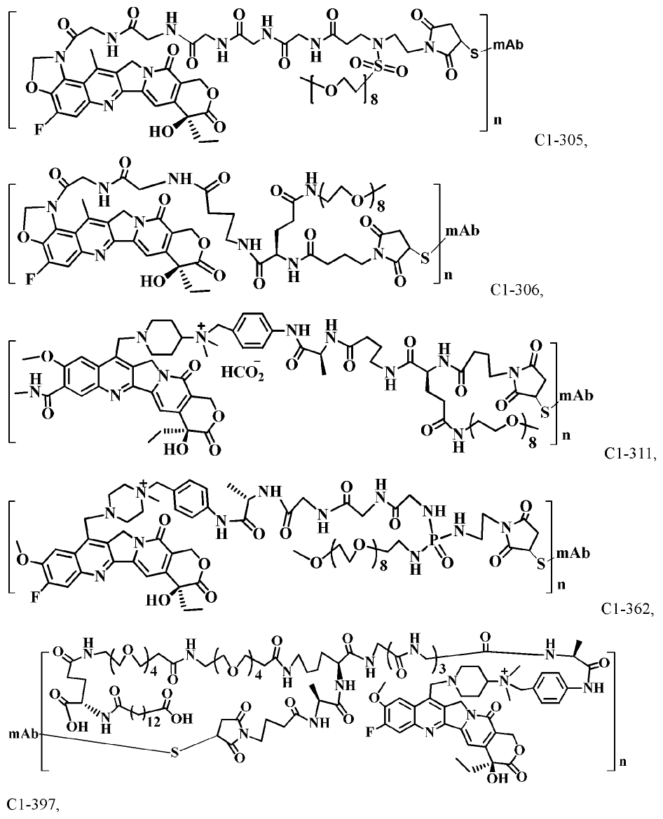

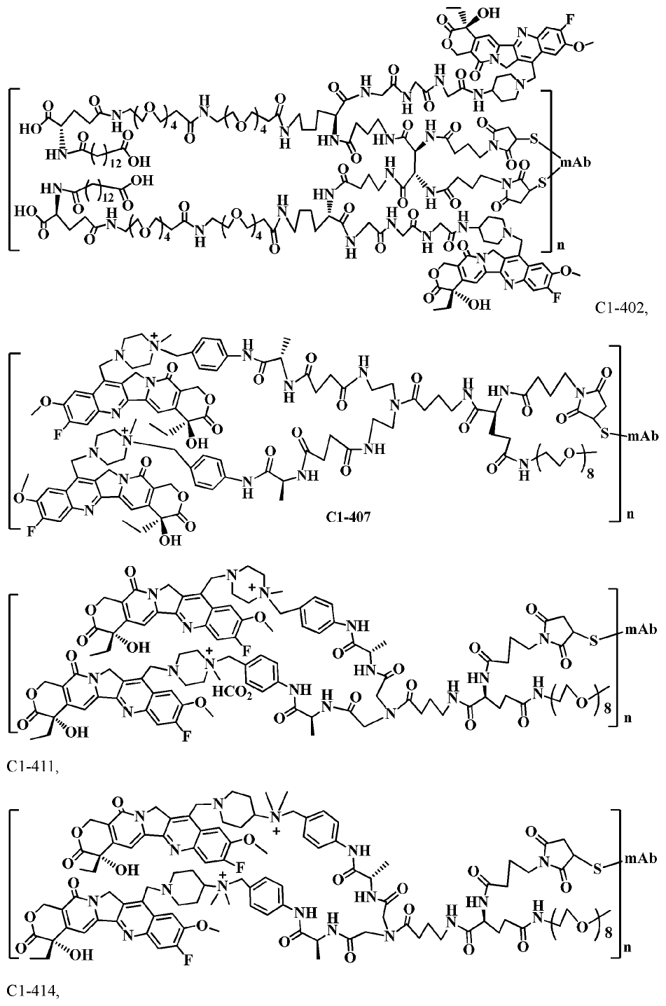

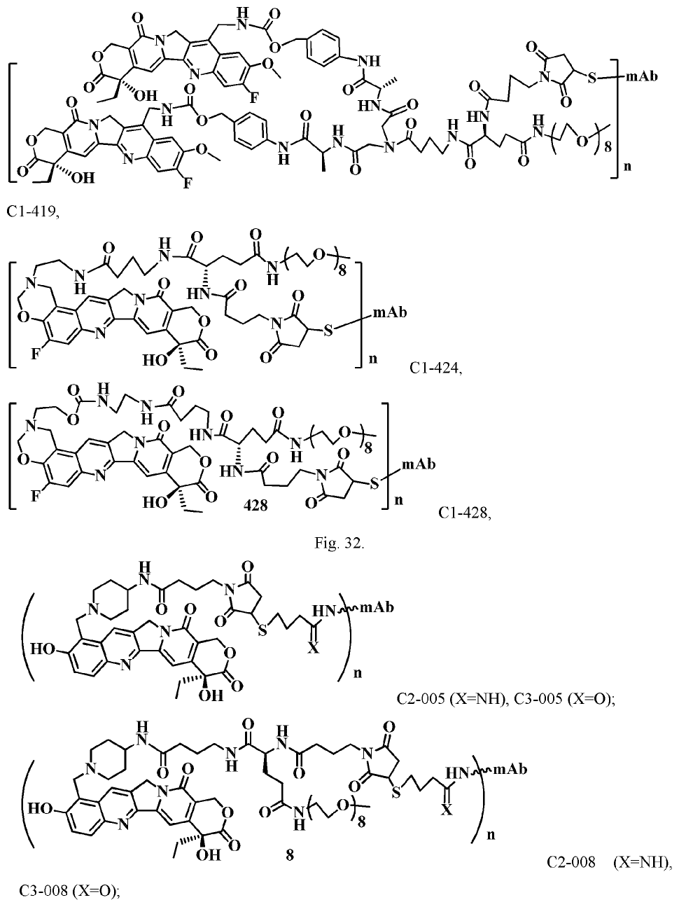

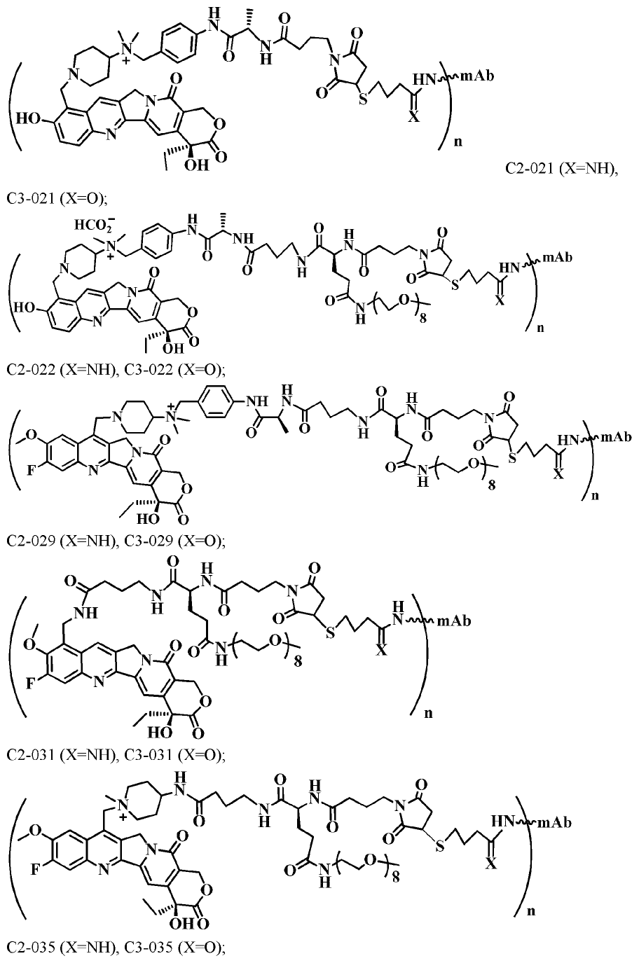

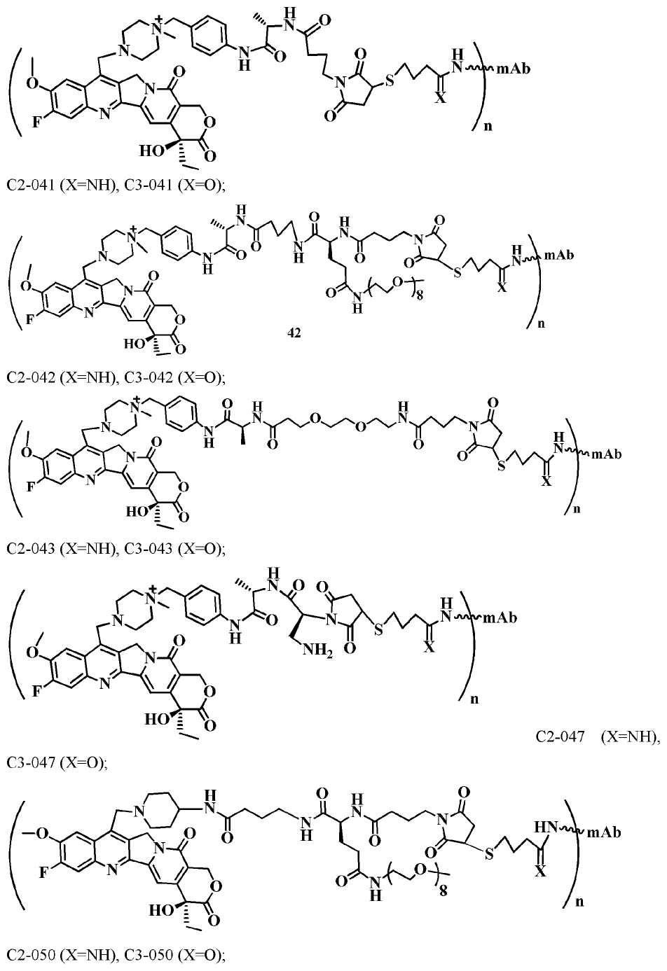

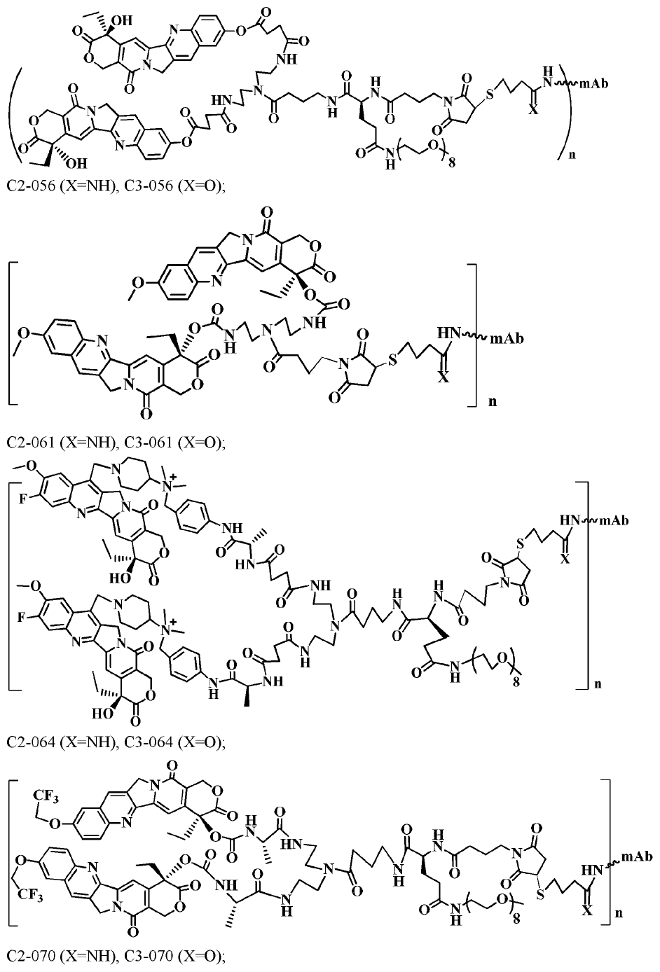

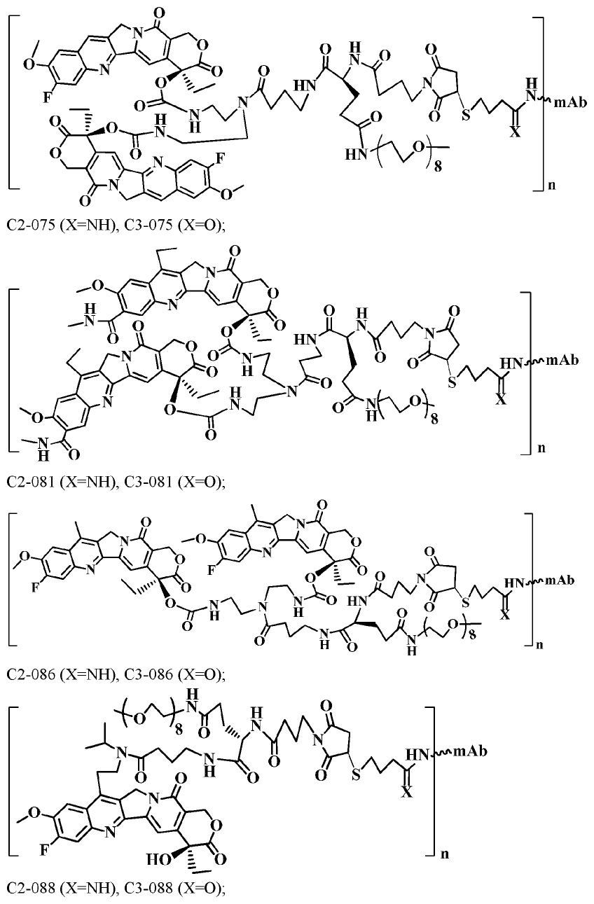

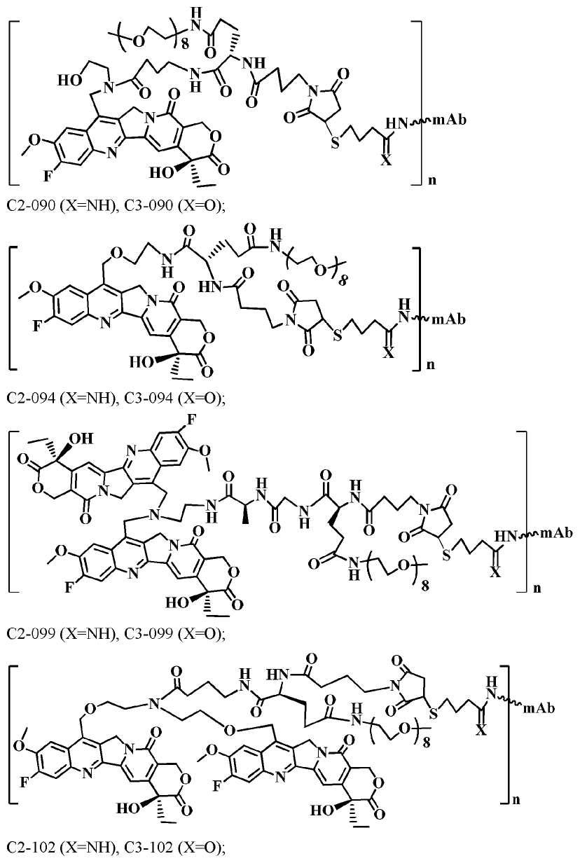

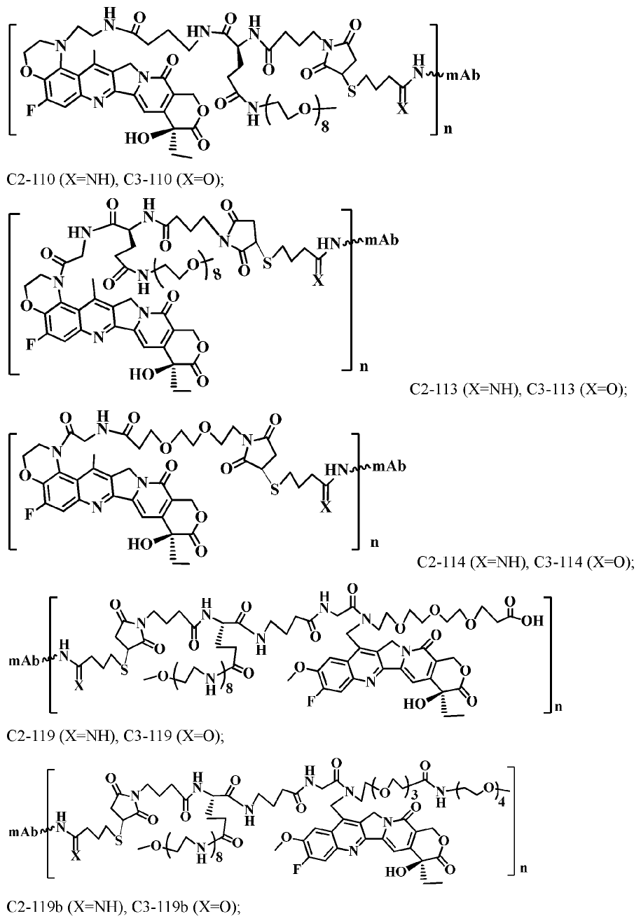

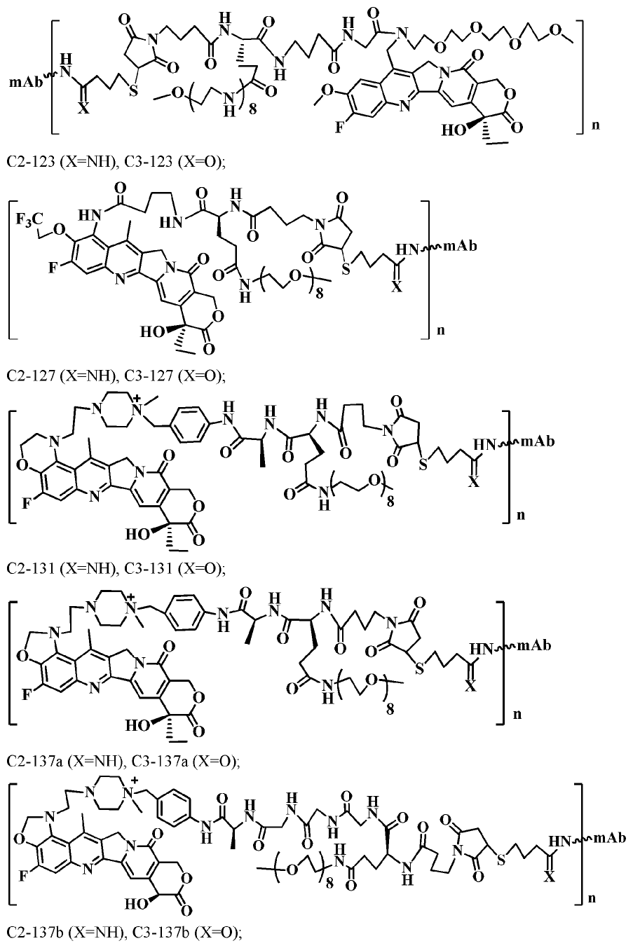

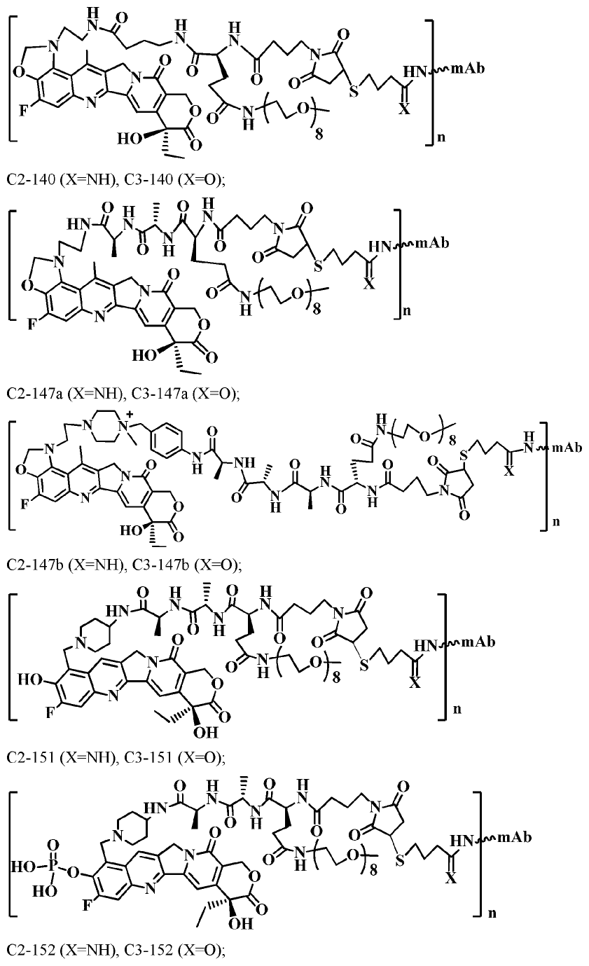

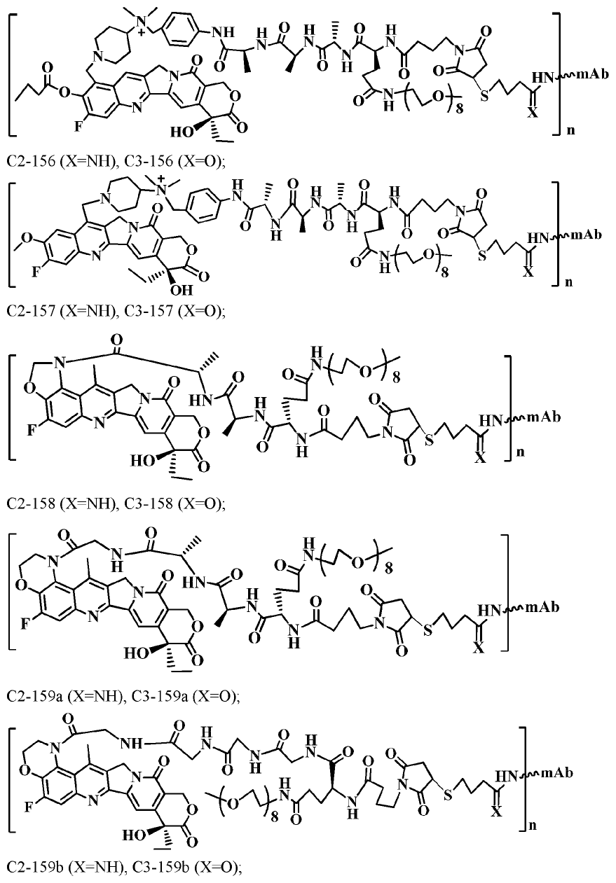

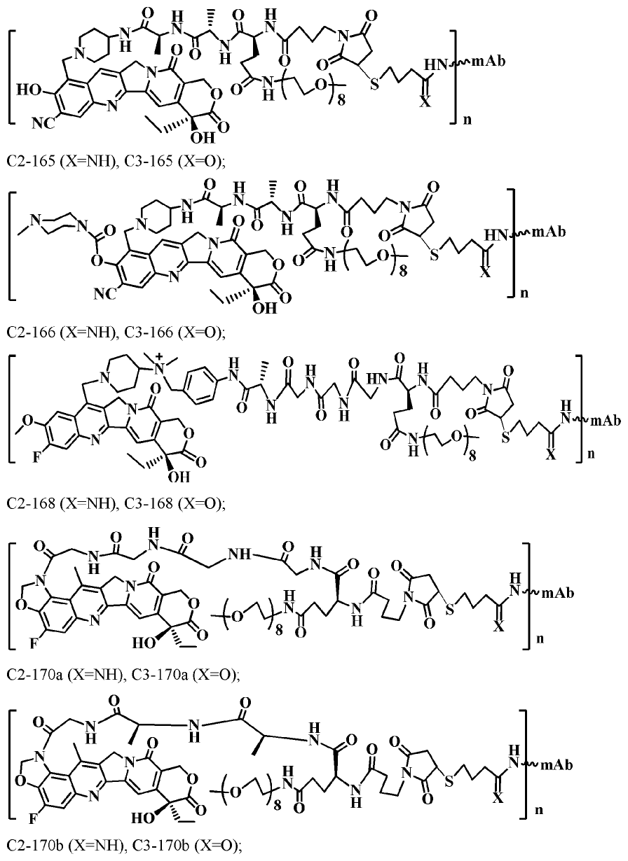

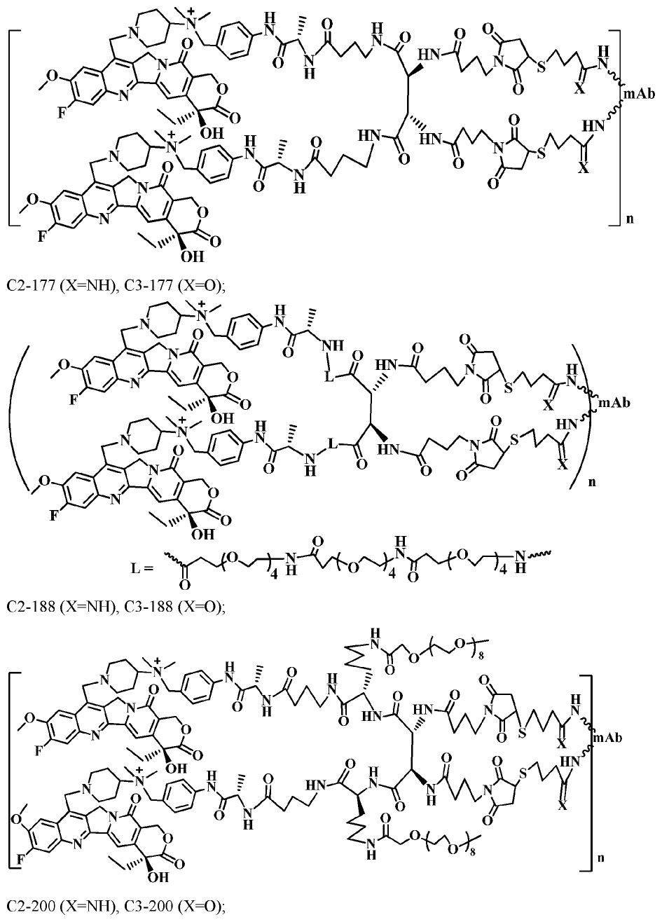

The structures of these conjugates are illustrated in the FIG. 32.

### Example 259. General method of Preparation of Conjugate C2-005, C3-005, C2-008, C3-008, C2-021, C3-021, C2-022, C3-022, C2-029, C3-029, C2-031, C3-031, C2-035, C3-035, C2-041, C3-041, C2-042, C3-042, C2-043, C3-043, C2-047, C3-047, C2-050, C3-050, C2-056, C3-056, C2-061, C3-061, C2-064, C3-064, C2-110, C3-110, C2-177, C3-177, C2-188, C3-188, C2-200, C3-200, C2-208, C3-208, C2-213, C3-213, C2-226, C3-226, C2-238, C3-238, C2-243, C3-243, C2-247, C3-247, C2-305, C3-305, C2-306, C3-306, C2-311, C3-311, C2-362, C3-362, C2-397, C3-397, C2-402, C3-402, C2-407, C3-407, C2-411, C3-411, C2-414, C3-414, C2-419, C3-419, C2-424, C3-424, C2-428, and C3-428

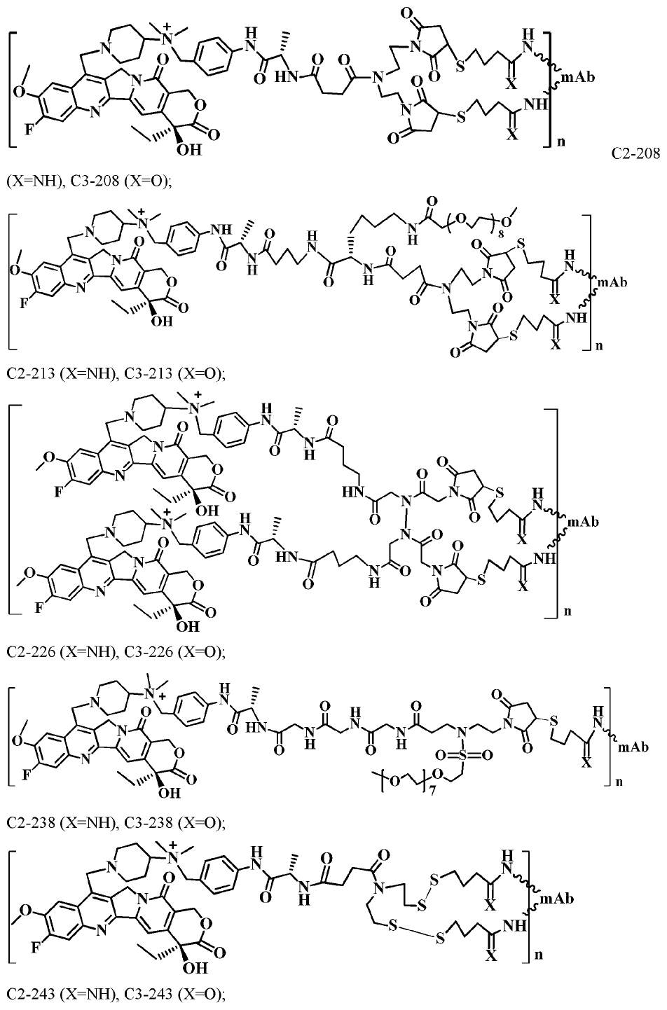

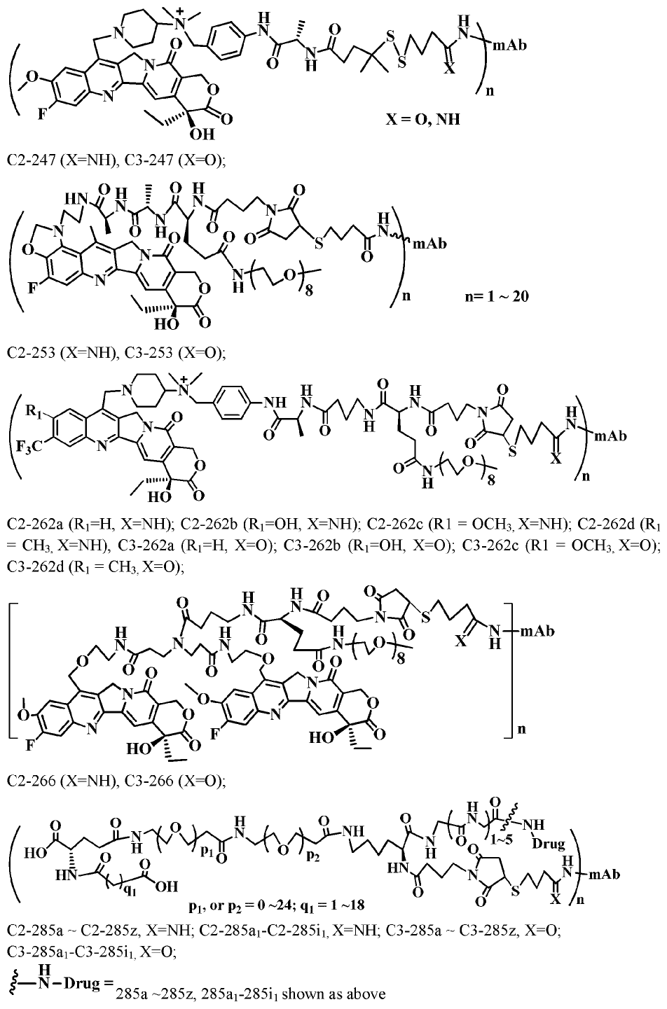

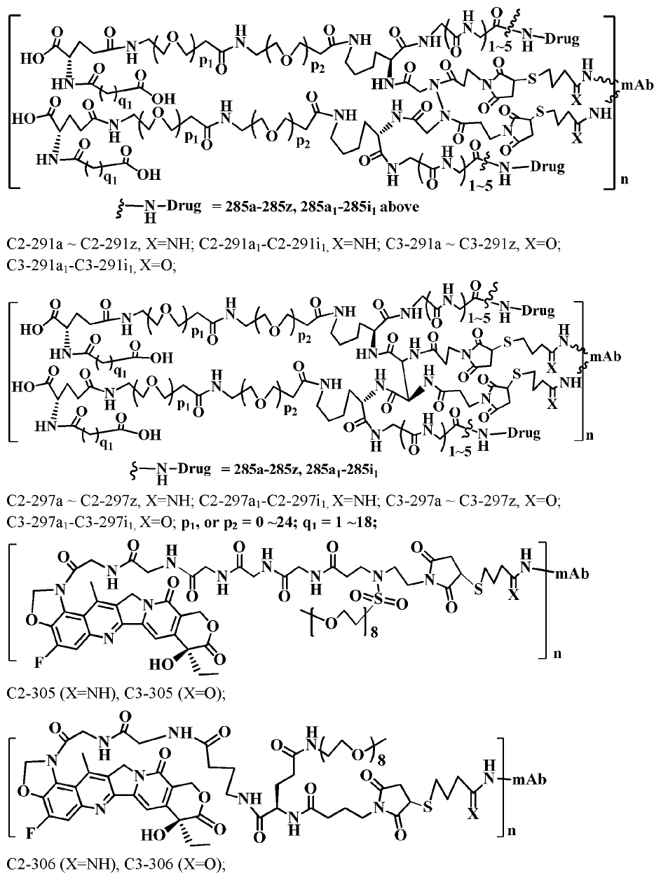

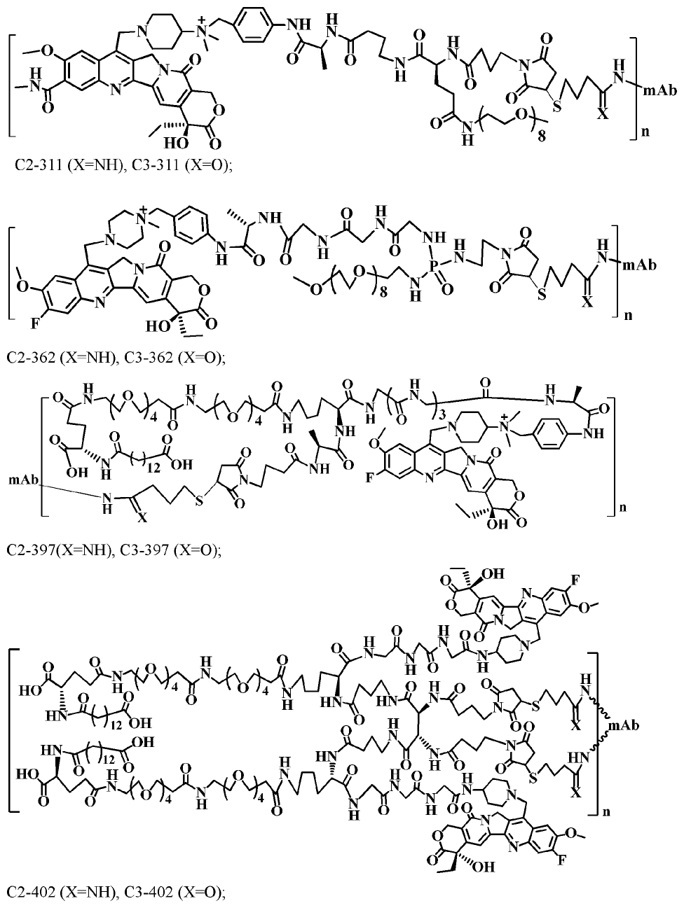

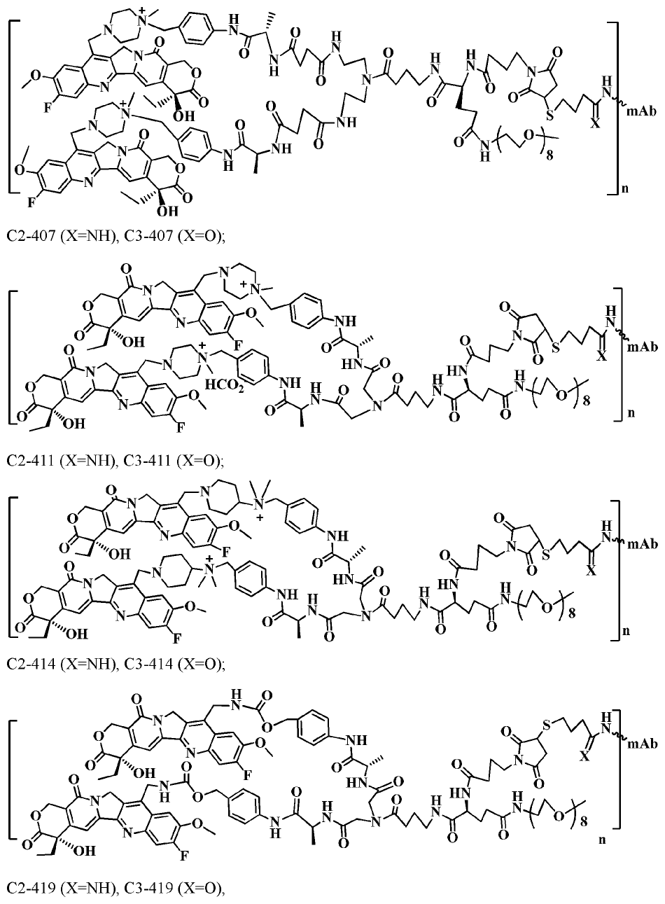

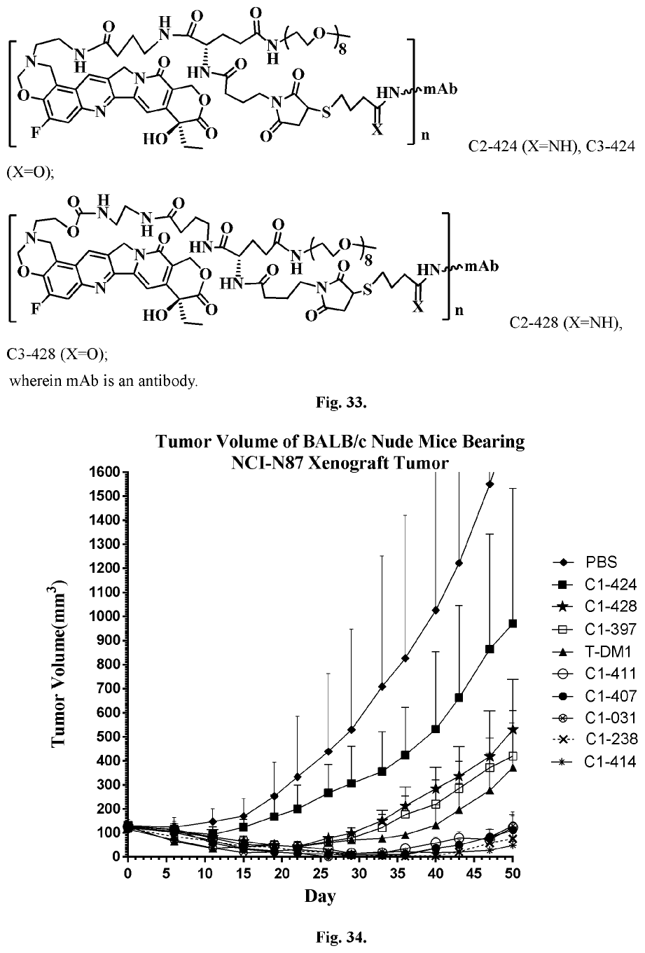

To a solution containing 2.0 mL of 10 mg/ml Her2 antibody (Herceptin) or Trop-2 antibody or EGFR antibody in pH 6.0-8.0 PBS buffer, 0.70˜2.0 mL of 100 mM NaH₂PO₄, pH 6.5˜8.5 buffers and Traut's reagent (2-iminothiolane HCl) (35-70 μL, 20 mM in water) or gamma-thiobutyrolactone (35-70 μL, 20 mM in DMA) respectively, the compound 5, 8, 21, 22, 29, 31, 35, 41, 42, 43, 47, 50, 56, 61, 64, 110, 177, 188, 200, 213, 226, 238, 243, 247, 305, 306, 311, 362, 397, 402, 407, 411, 414, 419, 424, and 428 (14-60 μL, 20 mM in DMA) were added independently, The mixture was incubated at RT for 4˜18 h, then purified on G-25 column eluted with 100 mM NaH₂PO₄, 50 mM NaCl pH 6.0˜7.5 buffer to afford 11.2˜18.5 mg of the conjugate compounds C2-005, C2-008, C2-021, C2-022, C2-029, C2-031, C2-035, C2-041, C2-042, C2-043, C2-047, C2-050, C2-056, C2-061, C2-064, C2-110, C2-177, C2-188, C2-200, C2-208, C2-213, C2-226, C2-238, C2-243, C2-247, C2-305, C2-306, C2-311, C2-362, C2-397, C2-402, C2-407, C2-411, C2-414, C2-419, C2-424, and C2-428 (85%˜98% yield with Traut's reagent) in 9.6-15.1 ml of the NaH₂PO₄, buffer, or C3-005, C3-008, C3-021, C3-022, C3-029, C3-031, C3-035, C3-041, C3-042, C3-043, C3-047, C3-050, C3-056, C3-061, C3-064, C3-110, C3-177, C3-188, C3-200, C3-208, C3-213, C3-226, C3-238, C3-243, C3-247, C3-305, C3-306, C3-311, C3-362, C3-397, C3-402, C3-407, C3-411, C3-414, C3-419, C3-424, and C3-428 (77%-94% yield with gamma-thiobutyrolactone) in 9.8-14.2 ml of the NaH₂PO₄, buffer. The drug/antibody ratio (DAR) was 4.5˜8.9, wherein DAR was determined via UPLC-QTOF mass spectrum and by UV (the CPT compounds here used the Extinction coefficient: E_{280 nm}=4992 M⁻¹ cm⁻¹; E_{377 nm}=16730 M⁻¹ cm⁻¹). It was 93˜99% monomer analyzed by SEC HPLC (SEC column was from Tosoh Bioscience, Tskgel G3000SW, 7.8 mm ID×30 cm, 0.5 ml/min, 100 min). The structures of these conjugates are illustrated in the FIG. 33.

### Example 260. In vitro cytotoxicity evaluation of C1-005, C1-008, C1-021, C1-022, C1-029, C1-031, C1-035, C1-041, C1-042, C1-043, C1-047, C1-050, C1-056, C1-061, C1-064, C1-110, C1-177, C1-188, C1-200, C1-208, C1-213, C1-226, C1-238, C1-243, C1-247, C1-305, C1-306, C1-311, C1-362, C1-397, C1-402, C1-407, C1-411, C1-414, C1-419, C1-424, C1-428, C2-005, C2-008, C2-021, C2-022, C2-029, C2-031, C2-035, C2-041, C2-042, C2-043, C2-047, C2-050, C2-056, C2-061, C2-064, C2-110, C2-177, C2-188, C2-200, C2-208, C2-213, C2-226, C2-238, C2-243, C2-247, C2-305, C2-306, C2-311, C2-362, C2-397, C2-402, C2-407, C2-411, C2-414, C2-419, C2-424, C2-428, C3-005, C3-008, C3-021, C3-022, C3-029, C3-031, C3-035, C3-041, C3-042, C3-043, C3-047, C3-050, C3-056, C3-061, C3-064, C3-110, C3-177, C3-188, C3-200, C3-208, C3-213, C3-226, C3-238, C3-243, C3-247, C3-305, C3-306, C3-311, C3-362, C3-397, C3-402, C3-407, C3-411, C3-414, C3-419, C3-424, and C3-428 (in comparison with T-DM1 when used Her2 antibody for the conjugation)

The cell line used in the cytotoxicity assays was NCI-N87, a human gastric carcinoma cell line and HCC827, non-small cell lung cancer cell line; The cells were grown in RPMI-1640 with 10% FBS. To run the assay, the cells (180 μl, 6000 cells) were added to each well in a 96-well plate and incubated for 24 hours at 37° C. with 5% CO₂. Next, the cells were treated with test compounds (20 μl) at various concentrations in appropriate cell culture medium (total volume, 0.2 mL). The control wells contain cells and the medium but lack the test compounds. The plates were incubated for 120 hours at 37° C. with 5% CO₂. MTT (5 mg/ml) was then added to the wells (20 μl) and the plates were incubated for 1.5 hr at 37° C. The medium was carefully removed and DMSO (180 μl) was added afterward. After it was shaken for 15 min, the absorbance was measured at 490 nm and 570 nm with a reference filter of 620 nm. The inhibition % was calculated according to the following equation: inhibition %=[1−(assay-blank)/(control-blank)]×100. The results are listed in Table 1.

TABLE 1
The Her2 antibody-CPT analog conjugates of
the patent application along with their cytotoxicity
IC₅₀ results against NCI-N87 cells:

| Conjugate | DAR (drug/ | Aggregation | IC50 (nM) against |
|---|---|---|---|
| compound | mAb ratio) | % | NCI-N87 cells |
| C1-005 | 7.3 | 5.5 | 42.8 |
| C1-008 | 7.5 | 3.1 | 33.6 |
| C1-021 | 7.6 | 1.2 | 31.2 |
| C1-022 | 7.4 | 0.2 | 23.6 |
| C1-029 | 6.9 | 0.4 | 0.81 |
| C1-031 | 7.3 | 0.8 | 0.14 |
| C1-035 | 7.4 | 0.5 | 1.91 |
| C1-041 | 7.4 | 0.9 | 2.83 |
| C1-042 | 7.5 | 0.4 | 1.74 |
| C1-043 | 7.6 | 0.6 | 3.46 |
| C1-047 | 7.7 | 0.7 | 4.82 |
| C1-050 | 7.6 | 1.0 | 1.23 |
| C1-056 | 8.5 | 4.2 | 22.2 |
| C1-061 | 7.2 | 6.1 | 93.9 |
| C1-064 | 8.3 | 0.4 | 1.18 |
| C1-110 | 7.8 | 1.6 | 0.86 |
| C1-177 | 7.2 | 2.1 | 1.11 |
| C1-188 | 7.3 | 1.3 | 1.18 |
| C1-200 | 7.4 | 0.4 | 0.82 |
| C1-208 | 7.2 | 0.8 | 0.96 |
| C1-213 | 7.2 | 0.5 | 1.01 |
| C1-226 | 7.9 | 0.7 | 0.69 |
| C1-238 | 8.0 | 0.1 | 0.16 |
| C1-243 | 7.2 | 0.6 | 0.86 |
| C1-247 | 8.0 | 0.7 | 0.51 |
| C1-305 | 7.9 | 0.7 | 0.58 |
| C1-306 | 7.9 | 0.8 | 0.42 |
| C1-311 | 8.0 | 0.2 | 0.57 |
| C1-362 | 8.0 | 0.1 | 0.44 |
| C1-397 | 7.9 | 0.1 | 0.57 |
| C1-402 | 8.2 | 0.2 | 0.31 |
| C1-407 | 10.6 | 0.2 | 0.14 |
| C1-411 | 10.7 | 0.2 | 0.53 |
| C1-414 | 10.8 | 0.2 | 0.15 |
| C1-419 | 10.2 | 1.6 | 0.55 |
| C1-424 | 7.9 | 0.9 | 0.69 |
| C1-428 | 7.9 | 0.7 | 0.61 |
| C2-005 | 7.8 | 5.7 | 39.1 |
| C2-008 | 8.0 | 3.1 | 35.3 |
| C2-021 | 8.0 | 1.3 | 32.7 |
| C2-022 | 7.9 | 0.2 | 21.1 |
| C2-029 | 7.8 | 0.3 | 0.70 |
| C2-031 | 7.9 | 0.7 | 0.21 |
| C2-035 | 8.0 | 0.6 | 0.73 |
| C2-041 | 7.9 | 1.0 | 1.13 |
| C2-042 | 8.0 | 0.4 | 0.91 |
| C2-043 | 7.9 | 0.6 | 1.28 |
| C2-047 | 7.9 | 0.7 | 1.77 |
| C2-050 | 7.8 | 1.1 | 1.09 |
| C2-056 | 8.8 | 4.1 | 32.3 |
| C2-061 | 7.9 | 5.9 | 87.1 |
| C2-064 | 8.0 | 0.3 | 0.42 |
| C2-110 | 7.9 | 1.7 | 0.93 |
| C2-177 | 8.4 | 2.2 | 1.27 |
| C2-188 | 8.1 | 1.4 | 0.83 |
| C2-200 | 8.5 | 0.5 | 0.61 |
| C2-208 | 7.5 | 0.7 | 0.47 |
| C2-213 | 4.2 | 0.5 | 1.18 |
| C2-226 | 7.3 | 0.6 | 0.93 |
| C2-238 | 7.6 | 0.2 | 0.15 |
| C2-243 | 3.8 | 0.6 | 0.71 |
| C2-247 | 6.8 | 0.6 | 0.48 |
| C2-305 | 7.2 | 0.6 | 0.43 |
| C2-306 | 7.6 | 0.7 | 0.37 |
| C2-311 | 7.5 | 0.1 | 0.63 |
| C2-362 | 7.8 | 0.1 | 0.51 |
| C2-397 | 7.6 | 0.1 | 0.59 |
| C2-402 | 7.2 | 0.2 | 0.38 |
| C2-407 | 10.4 | 0.3 | 0.15 |
| C2-411 | 10.6 | 0.2 | 0.55 |
| C2-414 | 10.8 | 0.2 | 0.14 |
| C2-419 | 10.5 | 0.7 | 0.48 |
| C2-424 | 7.2 | 0.8 | 0.62 |
| C2-428 | 7.2 | 0.6 | 0.58 |
| C3-005 | 7.9 | 6.0 | 42.9 |
| C3-008 | 8.0 | 3.3 | 37.3 |
| C3-021 | 8.0 | 1.2 | 33.9 |
| C3-022 | 8.0 | 0.3 | 23.7 |
| C3-029 | 7.9 | 0.4 | 0.91 |
| C3-031 | 8.0 | 0.7 | 0.20 |
| C3-035 | 8.0 | 0.5 | 0.69 |
| C3-041 | 7.9 | 0.8 | 1.35 |
| C3-042 | 8.0 | 0.5 | 0.97 |
| C3-043 | 7.9 | 0.6 | 1.11 |
| C3-047 | 8.0 | 0.7 | 1.61 |
| C3-050 | 7.8 | 1.1 | 0.92 |
| C3-056 | 8.2 | 4.3 | 30.1 |
| C3-061 | 8.0 | 6.2 | 73.7 |
| C3-064 | 8.8 | 0.4 | 0.31 |
| C3-110 | 8.0 | 1.7 | 0.81 |
| C3-177 | 8.2 | 2.3 | 1.05 |
| C3-188 | 8.0 | 1.4 | 0.91 |
| C3-200 | 8.2 | 0.4 | 0.82 |
| C3-208 | 7.2 | 0.8 | 0.51 |
| C3-213 | 4.8 | 0.5 | 1.33 |
| C3-226 | 7.8 | 0.8 | 0.89 |
| C3-238 | 8.0 | 0.1 | 0.14 |
| C3-243 | 4.0 | 0.7 | 1.27 |
| C3-247 | 7.2 | 0.6 | 0.73 |
| C3-305 | 7.9 | 0.7 | 0.55 |
| C3-306 | 7.8 | 0.9 | 0.41 |
| C3-311 | 7.8 | 0.2 | 0.71 |
| C3-362 | 8.0 | 0.1 | 0.48 |
| C3-397 | 7.9 | 0.1 | 0.50 |
| C3-402 | 8.2 | 0.2 | 0.33 |
| C3-407 | 10.5 | 0.2 | 0.18 |
| C2-411 | 10.3 | 0.2 | 0.48 |
| C2-414 | 10.0 | 0.2 | 0.29 |
| C2-419 | 10.2 | 1.6 | 0.60 |
| C2-424 | 7.8 | 1.0 | 0.63 |
| C2-428 | 7.9 | 0.6 | 0.53 |
| T-DM1 | 3.5 | 0.4 | 0.61 |

TABLE 2
The EGFR antibody-CPT analog conjugates of the
patent application along with their
cytotoxicity IC₅₀ results against HCC827 cells:

| Conjugate | DAR (drug/ | Aggregation | IC50 (nM) against |
|---|---|---|---|
| compound | mAb ratio) | % | HCC827 cells |
| C1-029 | 7.2 | 0.3 | 0.72 |
| C1-031 | 7.6 | 0.6 | 0.15 |
| C1-035 | 7.6 | 0.4 | 1.74 |
| C1-041 | 7.8 | 0.8 | 1.95 |
| C1-042 | 7.6 | 0.3 | 1.62 |
| C1-043 | 7.6 | 0.7 | 3.12 |
| C1-047 | 7.8 | 0.7 | 3.71 |
| C1-050 | 7.6 | 1.1 | 1.05 |
| C1-064 | 8.1 | 0.3 | 1.02 |
| C1-110 | 7.9 | 1.7 | 0.70 |
| C1-177 | 7.8 | 2.0 | 1.01 |
| C1-188 | 7.6 | 1.2 | 0.88 |
| C1-200 | 7.6 | 0.4 | 0.68 |
| C1-208 | 7.6 | 0.7 | 0.78 |
| C1-213 | 7.6 | 0.6 | 0.86 |
| C1-226 | 7.8 | 0.8 | 0.60 |
| C1-238 | 8.0 | 0.2 | 0.14 |
| C1-243 | 7.6 | 0.5 | 0.75 |
| C1-247 | 7.8 | 0.7 | 0.42 |
| C1-305 | 7.8 | 0.7 | 0.50 |
| C1-306 | 7.8 | 0.9 | 0.35 |
| C1-311 | 7.9 | 0.3 | 0.48 |
| C1-362 | 7.9 | 0.1 | 0.40 |
| C1-397 | 8.0 | 0.2 | 0.53 |
| C1-402 | 8.1 | 0.2 | 0.26 |
| C1-407 | 10.2 | 0.2 | 0.13 |
| C1-411 | 10.4 | 0.2 | 0.50 |
| C1-414 | 10.5 | 0.2 | 0.16 |
| C1-419 | 10.2 | 1.3 | 0.46 |
| C1-424 | 8.0 | 0.8 | 0.68 |
| C1-428 | 8.0 | 0.6 | 0.60 |

### Example 261. Antitumor Activity In Vivo (BALB/c Nude Mice Bearing N-87 Cell Xenograft Tumor) of Her2 Antibody-CPT Analog Conjugates

The in vivo efficacy of Her2 antibody conjugates of C1-031, C1-238, C1-397, C1-407, C1-411, C1-414, C1-424, C1-428, and T-DM1, were evaluated in a gastric carcinoma N-87 cell line tumor xenograft models. Five-week-old female BALB/c Nude mice (60 animals) were inoculated subcutaneously in the area under the right shoulder with N-87 carcinoma cells (5×10⁶ cells/mouse) in 0.1 mL of serum-free medium. The tumors were grown for 8 days to an average size of 130 mm³. The animals were then randomly divided into 11 groups (6 animals per group). The first group of mice served as the control group and was treated with the phosphate-buffered saline (PBS) vehicle. 9 groups were treated with conjugates C1-031, C1-238, C1-397, C1-407, C1-411, C1-414, C1-424, C1-428, and T-DM1, respectively at dose of 6 mg/Kg administered intravenously. Three dimensions of the tumor were measured every 3 or 4 days (twice a week) and the tumor volumes were calculated using the formula tumor volume=½(length×width height). The weight of the animals was also measured at the same time. A mouse was sacrificed when any one of the following criteria was met: (1) loss of body weight of more than 20% from pretreatment weight, (2) tumor volume larger than 1500 mm³, (3) too sick to reach food and water, or (4) skin necrosis. A mouse was considered to be tumor-free if no tumor was palpable.

The antitumor activity results were plotted in FIG. 34. All the 10 conjugates did not cause the animal body weight loss at dose of 6.0 mg/Kg. All conjugates demonstrated antitumor activity as comparison with PBS buffer.

TABLE 3
The inhibition of the tumor growth at dose of 6 mg/Kg is

| Conjugate | Tumor growth delay |
|---|---|
| C1-424 | 10 days |
| C1-428 | 25 days |
| C1-397 | 26 days |
| T-DM1 | 32 days |
| C1-414 | 43 days |
| C1-407 | 43 days |
| C1-031 | 43 days |
| C1-238 | >43 days |
| C1-414 | >43 days |

The order of in vivo antitumor ability is C1-424<C1-428<C1-397<T-DM1<C1-411<C1-407<C1-031<C1-238<C1-414.

### Example 262. Toxicity Study of the Her2 Antibody-CPT Analog Conjugates in Comparison with T-DM1

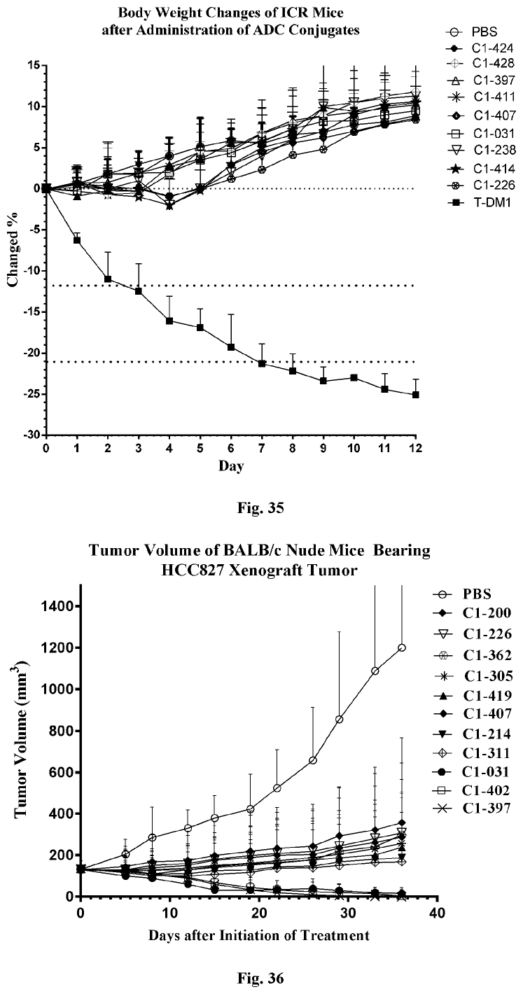

Change (typically reduction) in body weight (BW) is animal's general response to drug toxicities. 66 female ICR mice, 6-7 weeks old, were separated into 11 groups. Each group included 6 mice and each mouse was given conjugates C1-031, C1-226, C1-238, C1-397, C1-407, C1-411, C1-414, C1-424, C1-428, and T-DM1, respectively at dose of 150 mg/Kg per mouse, i.v. bolus. A control group (n=8) was set by I.V. dosing vehicle solution, phosphate buffered saline (PBS). The toxicity results were plotted in FIG. 35. BW of the control group, and the group of conjugates C1-031, C1-397, C1-407, C1-411, C1-424, and C1-428, at doses of 150 mg/Kg was not reduced in 12-days experiment. BW of the rest conjugates C1-226, C1-238, C1-414 and T-DM1 at doses of 150 mg/Kg, was reduced during 12-days experiment and the highest degrees of about 2% BW loss was seen for C1-226, C1-238, and C1-414 on day 5. The BW reduction in all tested CPT conjugates was much less than that of T-DM1. In contrast, BW in T-DM1 group continued decreasing with a maximal reduction of 25% from pre-dosing value, and no recovery tendency was seen at the end of the study. The BW change experiments demonstrated greater tolerability for these CPT conjugates than that of T-DM1 in these mice.

### Example 263. Antitumor Activity In Vivo (BALB/c Nude Mice Bearing HCC827 Cell Xenograft Tumor) of EGFR Antibody-CPT Analog Conjugates

The in vivo efficacy of EGFR antibody conjugates of C1-031, C1-200, C1-214, C1-226, C1-305, C1-306, C1-311, C1-362, C1-402, C-407 and C1-419, were evaluated in a non-small cell lung carcinoma HCC827 cell line tumor xenograft model. Five-week-old female BALB/c Nude mice (72 animals) were inoculated subcutaneously in the area under the right shoulder with N-87 carcinoma cells (5×10⁶ cells/mouse) in 0.1 mL of serum-free medium. The tumors were grown for 8 days to an average size of 130 mm³. The animals were then randomly divided into 12 groups (6 animals per group). The first group of mice served as the control group and was treated with the phosphate-buffered saline (PBS). 11 groups were treated with conjugates C1-031, C1-200, C1-226, C1-214, C1-305, C1-311, C1-362, C1-397, C1-402, C1-407, and C1-419 in PBS respectively at dose of 6 mg/Kg at concentration 3.2˜8.0 mg/ml administered intravenously.

Three dimensions of the tumor were measured every 3 or 4 days (twice a week) and the tumor volumes were calculated using the formula tumor volume=½(length×width×height). The weight of the animals was also measured at the same time. A mouse was sacrificed when any one of the following criteria was met: (1) loss of body weight of more than 20% from pretreatment weight, (2) tumor volume larger than 1500 mm³, (3) too sick to reach food and water, or (4) skin necrosis. A mouse was considered to be tumor-free if no tumor was palpable.

The antitumor activity results were plotted in FIG. 36. All the 11 conjugates did not cause the animal body weight loss at dose of 6.0 mg/Kg. All conjugates demonstrated antitumor activity as comparison with PBS buffer.

TABLE 4
The inhibition of the tumor growth at dose of 6 mg/Kg is

| Conjugate | Tumor growth delay |
|---|---|
| C1-200 | 20 days |
| C1-226 | 22 days |
| C1-362 | 23 days |
| C1-305 | 24 days |
| C1-419 | 25 days |
| C1-407 | 27 days |
| C1-214 | 28 days |
| C1-311 | 30 days |
| C1-031 | >36 days |
| C1-402 | >36 days |
| C1-397 | >36 days |

The order of in vivo antitumor ability is C1-200<C1-226<C1-362<C1-305<C1-419<C1-407<C1-214<C1-311<C1-031<C1-402<C1-397.

1-21. (canceled)

22. A cell surface-binding molecule-camptothecin analog conjugate having Formula (I) below: 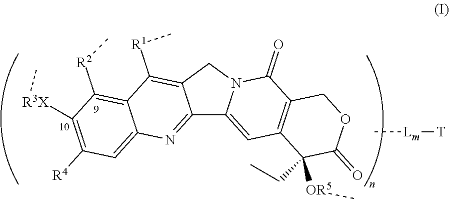
or a pharmaceutically acceptable salt, hydrate, or hydrated salt thereof; or a polymorphic crystalline structure thereof; or an isotope, optical isomer, racemate, diastereomer or enantiomer thereof;
wherein T is a cell-binding molecule; L is a releasable linker; ----- is a linkage bond that L connects to an atom of R¹, R², R³ or R⁵ independently inside the bracket independently; n is 1-30; and m is 1-10;
inside the bracket is an amptothecin analog wherein:
R¹ and R² are independently H; linear or branched C₁-C₆ alkyl, alkyl alcohol, alkyl amine (including primary, secondary, tertiary amine, or quaternary ammonium), aminoalkyl, oxylalkyl, aminoalkylamino, oxylalkylamino, aminoalkyloxyl, oxylalkyloxyl, alkyl carboxylic acid, or carbonyl; C₂-C₆ heteroalkyl, alkylcycloalkyl, heterocycloalkyl, heterocyclic, aminocycloalkyl, heteroalkylcycloalkyl, alkylcarbonyl, aminoalkylcarbonyl, oxylalkylcarbonyl, alkyl ether, alkyl ester, alkyl amide, oxylalkylamide, aminoalkylamide, oxime; NH₂, or OH;
R³ is independently H, C(O)NH, C(O)O, SO₂R⁶, SO₃R⁶, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR)₂, C(O)R⁶, C(O)NHR⁶; linear or branched C₁-C₆ alkyl, alkyl alcohol, alkyl amine (including primary, secondary, tertiary amine, or quaternary ammonium), or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide, or oxime; C₅-C₁₂ glycoside, NH₂, or OH;
R⁴ is F, Cl, Br, I, CN, NO₂, SO₃H, OR⁶, SR⁶, S(O₂)R⁶, NHR⁶, N(R⁶)(R^{6′}), C(O)XR⁶, or N⁺(R⁶)(R^{6′})(R^{6″});
X is NH or O;
R⁵ is H, C(O)O, C(O)NH, R⁶C(O), linear or branched C₁-C₆ alkyl, alkyl alcohol, alkyl amine (including primary, secondary, tertiary amine, or quaternary ammonium), or alkyl carboxylic acid; C₂-C₆ carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;
R⁶, R^{6′}, and R^{6″} are independently H, C₁-C₆ alkyl, alkyl alcohol, alkyl amine (including primary, secondary, tertiary amine, or quaternary ammonium) or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or a pharmaceutical salt;
or R¹, R², R³ and R⁶ can be independently absent, and R², R³, X, C-10 and C-9 can join together to form a 5-, 6- or 7-member heterocyclic ring;
T is selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and/or a cell-binding peptide, protein, or small affinity molecule attached or coated on an albumin, a polymer, a dendrimer, a liposome, a nanoparticle, a vesicle, or on a (viral) capsid;
L has the formula of: -W_{w}-(Aa)ᵣ-Vᵥ-, wherein: -W- is a Stretcher unit; w is 0 or 1; each -Aa- is independently an amino acid unit; r is independently an integer ranging from 0 to 12; -V- is a Spacer unit; and v is 0, 1 or 2;
-W-, when present, links T to -Aa-, or to V when Aa is not present; W linked to T has one of structures below: 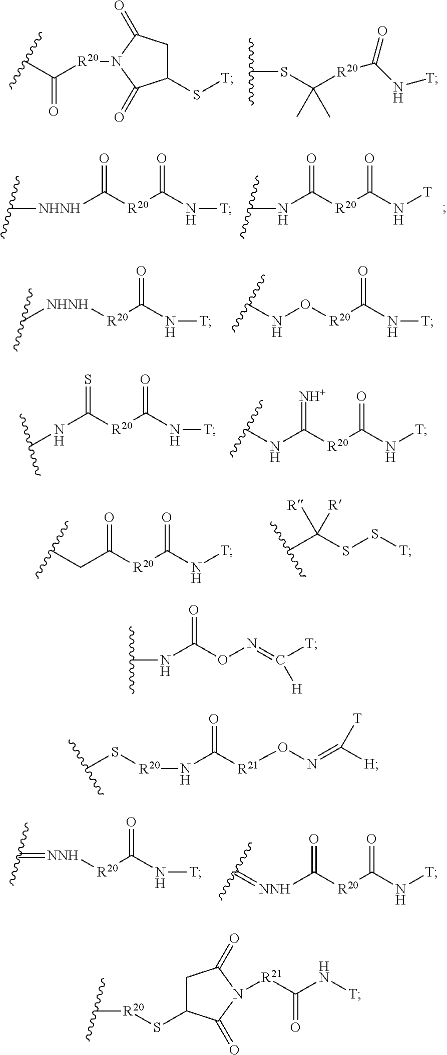 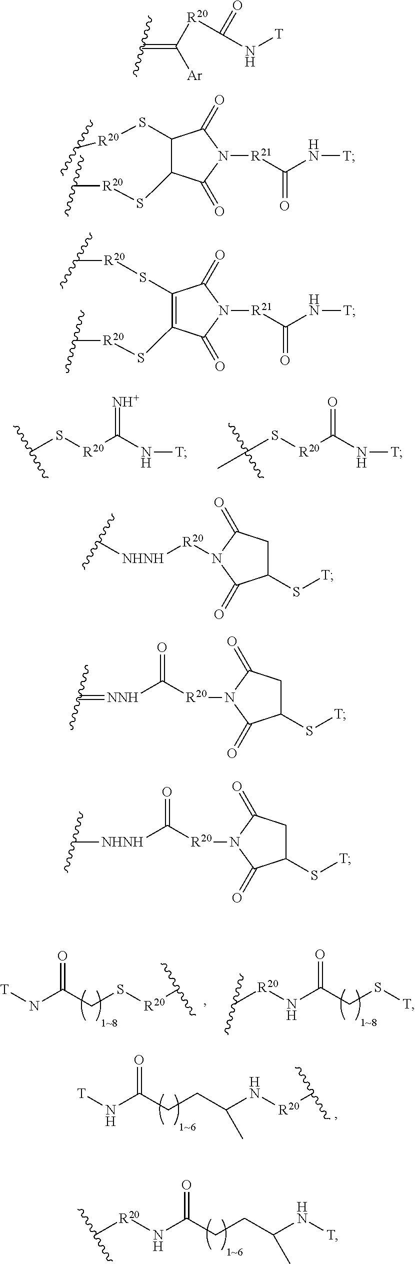 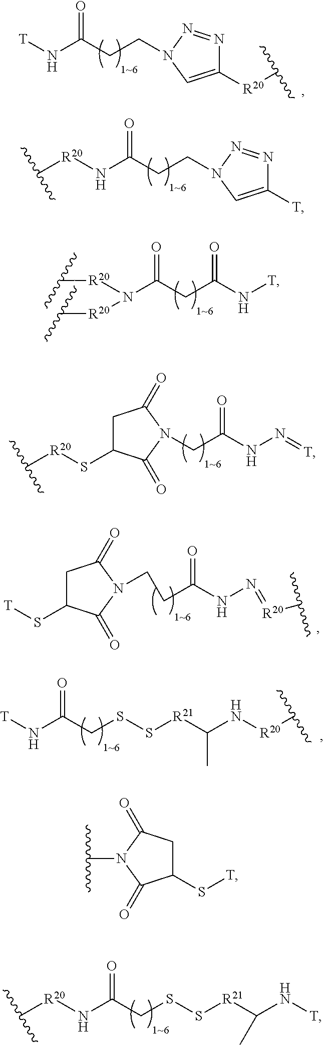 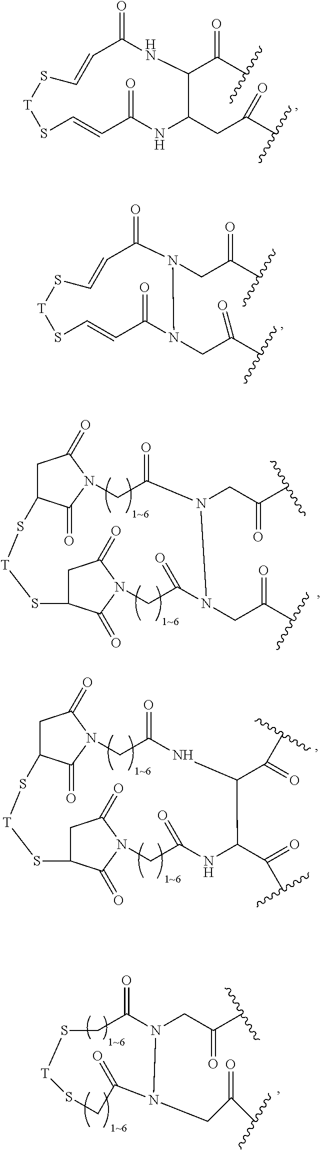 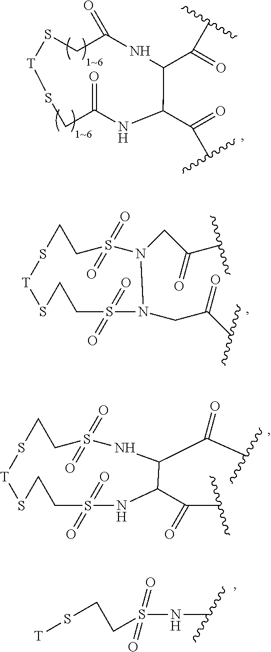
wherein R²⁰ and R²¹ are selected from C₁-C₈ alkyl, —C₁-C₇ carbocyclo, —O—(C₁-C₈ alkyl)-, -arylene, —C₁-C₈ alkylene-arylene, -arylene, —C₁-C₈ alkylene, —C₁-C₈ alkylene-(C₁-C₃ carbocyclo)-, —(C₃-C₇ carbocyclo)-C₁-C₉ alkylene-, —C₃-C₈ heterocyclo-, —C₁-C₈ alkylene-(C₃-C₃ heterocyclo)-, —(C₃-C₈ heterocyclo)-C₁-C₉ alkylene-, —(CH₂CH₂O)ₖ—, —(CH(CH₃)CH₂O)ₖ—, and —(CH₂CH₂O)ₖ—CH₂—; k is an integer ranging from 1-20; R′ and R″ are independently H or CH₃;
-(Aa)r- is a natural or unnatural amino acid, the same or different amino acid sequences of dipeptide, tripeptide, tetrapeptide, pentapeptide, hexapeptide, heptapeptide, octapeptide, nonapeptide, decapeptide, undecapeptide or dodecapeptide unit, and r is an integer ranging from 0 to 12;
-V- is either a self-immolative or a non self-immolative unit, the self-immolative unit includes para-aminobenzyl-carbamoyl (PAB) group, 2-aminoimidazol-5-methanol derivatives, heterocyclic PAB analogs, beta-glucuronide, and ortho or para-aminobenzylacetals; or one of following structures: 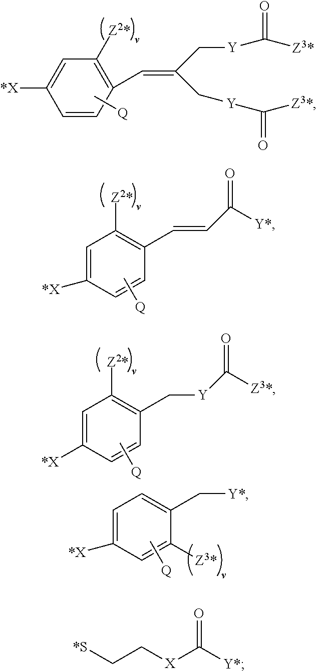
wherein the (*) atom is a point of attachment of additional spacer or releasable linker unit, amino acid (Aa)ᵣ, camptothecin analog, and/or the binding molecule (T); X, Y and Z³ are independently NH, O, or S; Z² is H, NH, O or S independently; v is 0 or 1; Q is independently H, OH, C₁-C₆ alkyl, (OCH₂CH₂)ₙ, F, Cl, Br, I, OR¹⁷, or SR¹⁷, NR¹⁷R¹⁸, N═NR¹⁷, N═R¹⁷, NR¹⁷R¹⁸, NO₂, SOR¹⁷R¹⁸, SO₂R¹⁷, SO₃R¹⁷, OSO₃R¹⁷, PR¹⁷R¹⁸, POR¹⁷R¹⁸, PO₂R¹⁷R¹⁸, OPO(OR¹⁷)(OR¹⁸), or OCH₂PO(OR¹⁷(OR¹⁸), wherein R¹⁷, R¹⁸ are independently H, C₁-C₈ alkyl; C₂-C₈ alkenyl, alkynyl, or heteroalkyl; C₃-C₈ aryl, heterocyclic, carbocyclic, cycloalkyl, heterocycloalkyl, heteroaralkyl, alkylcarbonyl; or pharmaceutical cation salt; v is an integer ranging from 1-20;
the non-self-immolative spacer linker units (-V-) include: 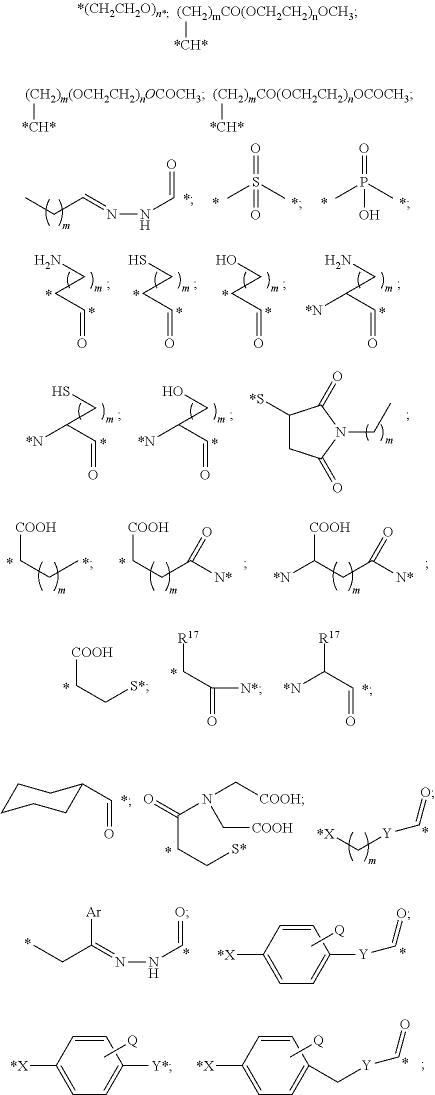 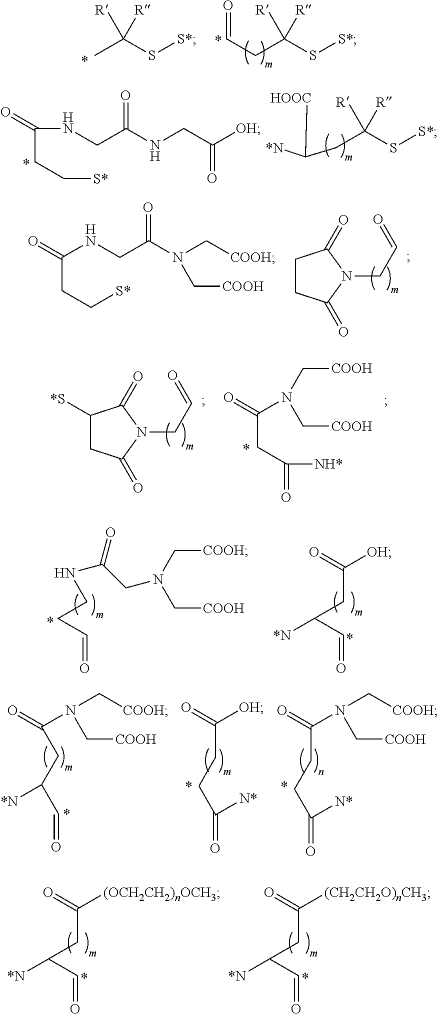 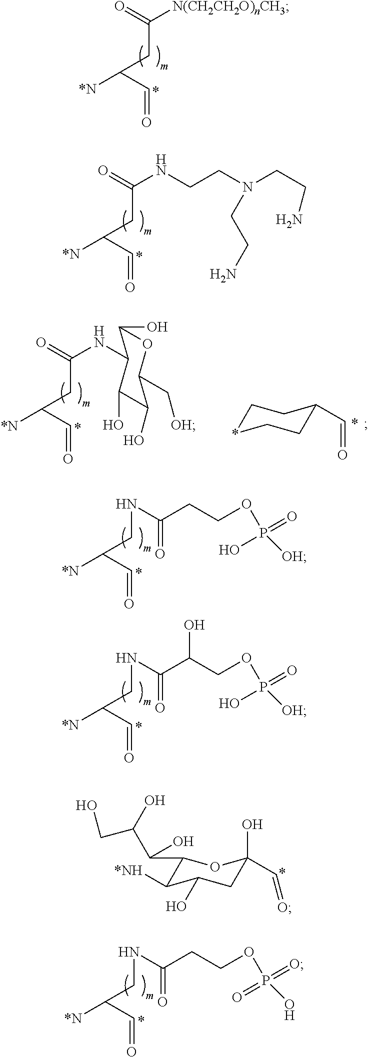 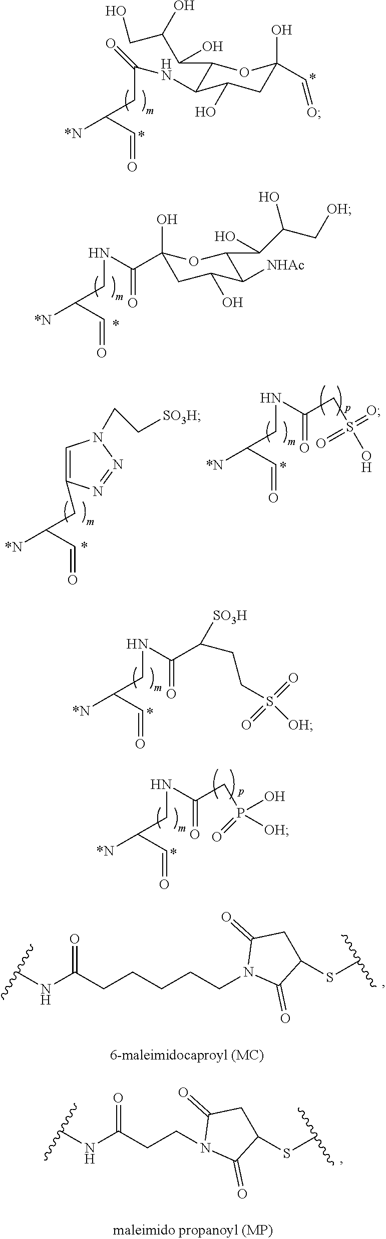 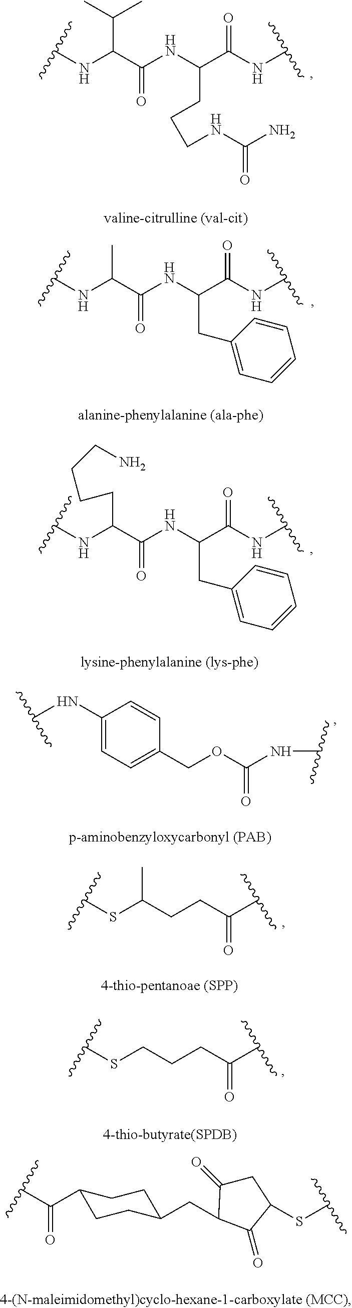 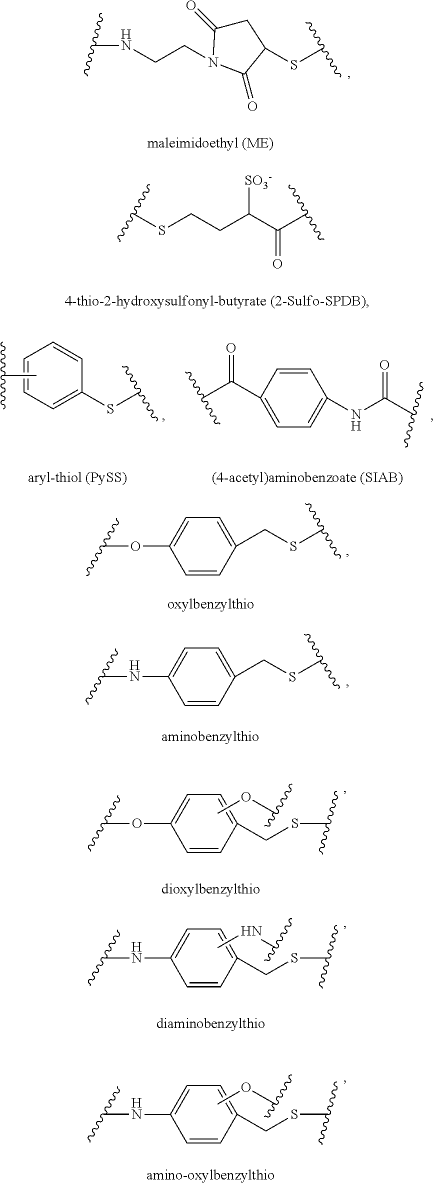 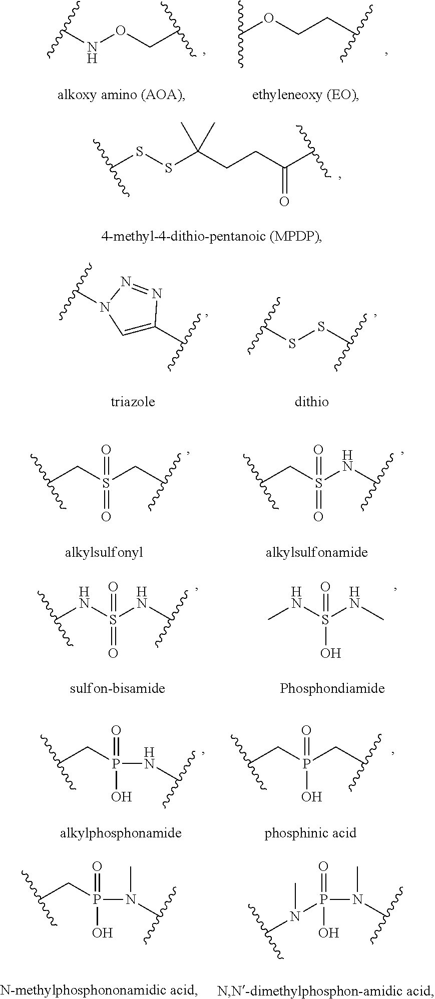 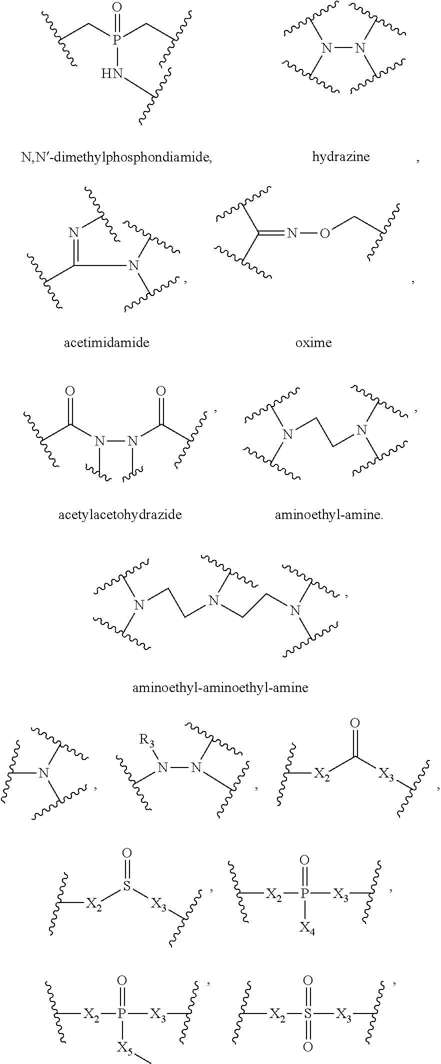 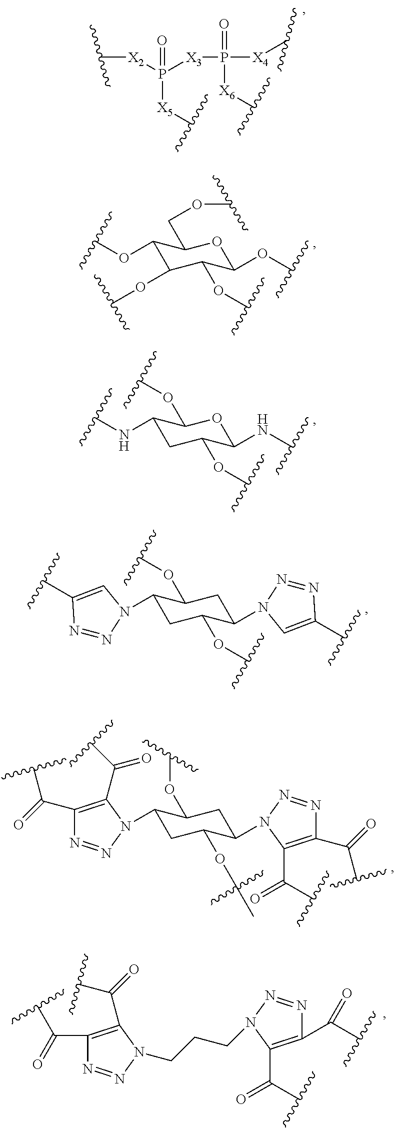 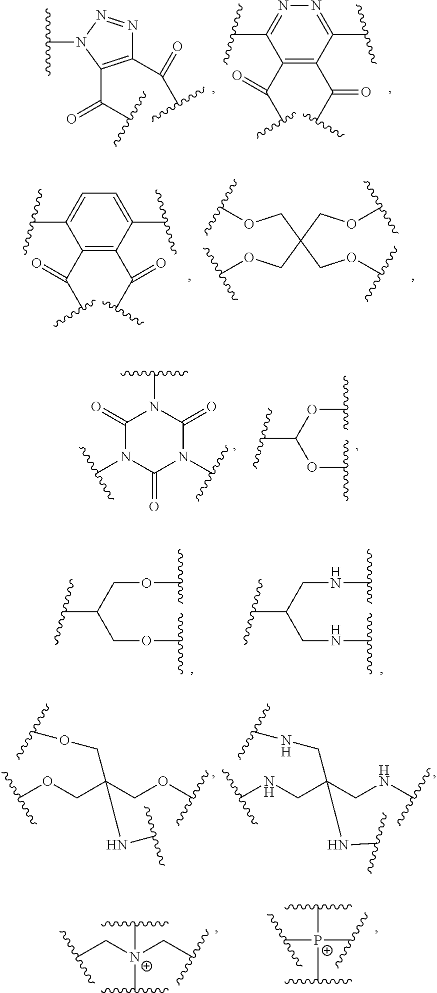 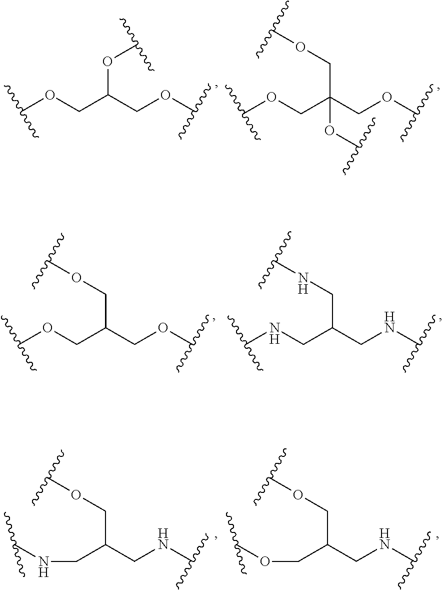 [SMILES: *OCC(CO*)O* ; *OCC(CO*)(CO*)O* ; *OCC(CO*)CO* ; *NCC(CN*)CN* ; *NCC(CN*)CO* ; *NCC(CO*)CO*]
or L- or D-, natural or unnatural peptides containing 1-20 the same or different amino acids;
wherein “*” and “ [symbol] ” are points of attachment of additional spacer or releaseable linkers, the camptothecin analogs, and/or the binding molecules; m is 1-10; n is 1-20; X₂, X₃, X₄, X₅, or X₆ are independently NH; NHNH; N(R₁₂); N(R₁₂)N(R_{12′}); O; S; C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; CH₂OR₁₂, CH₂SR₁₂, CH₂NHR₁₂, or 1-8 amino acids; wherein R₁₂ and R_{12′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₁-C₈ ester, ether, or amide; or polyethyleneoxy unit of formula (OCH₂CH₂)ₚ or (OCH₂CH(CH₃))ₚ, wherein p is an integer from 0 to about 1000.

23. The conjugate according to claim 22, having Formula (II): 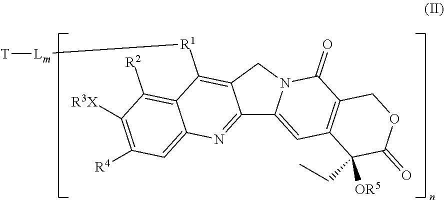
or a pharmaceutically acceptable salt, hydrate, or hydrated salt thereof; or a polymorphic crystalline structure thereof; or an isotope, optical isomer, racemate, diastereomer or enantiomer thereof;
wherein T is a targeting or binding ligand; L is a releasable linker; n is 1-30 and m is 1-10;
inside the bracket is a camptothecin analog wherein:
R¹ is linear or branched C₁-C₆ alkyl, alkyloxyl, alkyl amino (including primary, secondary, tertiary amino, or quaternary ammonium), oxylcarbonyl, aminocarbonyl, aminoalkyl, oxylalkyl, aminoalkylamino, oxylalkylamino, aminoalkyloxyl, oxylalkyloxyl, or alkyl carboxylic; C₂-C₆ heteroalkyl, alkylcycloalkyl, heterocycloalkyl, heterocyclic, oxylcycloalkyl, aminocycloalkyl, heteroalkylcycloalkyl, alkylcarbonyl, aminoalkylcarbonyl, oxylalkylcarbonyl, alkyl ether, alkyl ester, alkyl amide, oxylalkylamide, aminoalkylamide, oxime; NH, or O;
R² is H, linear or branched C₁-C₆ alkyl, alkyl alcohol, alkyl amine (including primary, secondary, tertiary amine, or quaternary ammonium), aminoalkyl alcohol, aminoalkyl amine, oxylalkyl alcohol, oxylalkyl amine, aminoalkyl, oxylalkyl, or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide, or oxime; NH₂, or OH;
R³ is independently H, R⁶NHC(O), R⁶OC(O), SO₂R⁶, SO₃R, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR^{6′})₂, R⁶C(O), C(O)NR⁶R^{6′}; linear or branched C₁-C₆ alkyl, alkyl alcohol, alkyl amine (including primary, secondary, tertiary amine, or quaternary ammonium), or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide, or oxime; or C—C₁₂ glycoside;
R⁴ is F, Cl, Br, I, CN, NO₂, SO₃H, OR⁶, SR⁶, S(O₂)R⁶, NHR⁶, N(R⁶)(R^{6′}), C(O)XR⁶, or N⁺(R⁶)(R^{6′})(R^{6″});
X is NH or O;
R⁵ is H, C(O)OR⁶, C(O)NHR⁶, R⁶C(O), linear or branched C₁-C₆ alkyl, alkyl alcohol, alkyl amine (including primary, secondary, tertiary amine, or quaternary ammonium), or alkyl carboxylic acid; C₂-C₆ carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;
R⁶, R^{6′}, and R^{6″} are independently H, C₁-C₆ alkyl, alkyl alcohol, alkyl amine (including primary, secondary, tertiary amine, or quaternary ammonium) or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or a pharmaceutical salt;
or R¹ can be absent and C-7 directly links to L, and R², R³, X, C-10 and C-9 can join together to form a 5-, 6- or 7-member heterocyclic ring.

24. The conjugate according to claim 22, having Formula (III): 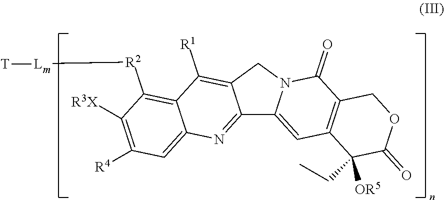
or a pharmaceutically acceptable salt, hydrate, or hydrated salt thereof; or a polymorphic crystalline structure thereof; or an isotope, optical isomer, racemate, diastereomer or enantiomer thereof;
wherein T is a targeting or binding ligand; L is a releasable linker; n is 1-30; and m is 1-10;
inside the bracket is a camptothecin analog wherein:
R¹ is linear or branched C₁-C₆ alkyl, alkyloxyl, alkyl amino (including primary, secondary, tertiary amino, or quaternary ammonium), oxylcarbonyl, aminocarbonyl, aminoalkyl, oxylalkyl, aminoalkylamino, oxylalkylamino, aminoalkyloxyl, oxylalkyloxyl, or alkyl carboxylic; C₂-C₆ heteroalkyl, alkylcycloalkyl, heterocycloalkyl, heterocyclic, oxylcycloalkyl, aminocycloalkyl, heteroalkylcycloalkyl, alkylcarbonyl, aminoalkylcarbonyl, oxylalkylcarbonyl, alkyl ether, alkyl ester, alkyl amide, oxylalkylether, aminoalkylether, oxylalkylester, aminoalkylester, oxylalkylamide, aminoalkylamide, or oxime; NH, or O;
R² is NH, NR⁶, N⁺R⁶R^{6′}, O, S, linear or branched C₁-C₆ alkyl, alkyl alcohol, alkyl amine (including primary, secondary, tertiary amine, or quaternary ammonium), aminoalkyl alcohol, aminoalkyl amine, oxylalkyl alcohol, oxylalkyl amine, aminoalkyl, oxylalkyl, or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide, oxime; oxylalkylether, aminoalkylether, oxylalkylester, aminoalkylester, oxylalkylamide, or aminoalkylamide;
R³ is independently H, R⁶NHC(O), R⁶OC(O), SO₂R⁶, SO₃R, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR^{6′})₂, R⁶C(O), C(O)NR⁶R^{6′}; linear or branched C₁-C₆ alkyl, alkyl alcohol, alkyl amine (including primary, secondary, tertiary amine, or quaternary ammonium), or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide, or oxime; or C—C₁₂ glycoside;
R⁴ is F, Cl, Br, I, CN, NO₂, SO₃H, OR⁶, SR⁶, S(O₂)R⁶, NHR⁶, N(R⁶)(R^{6′}), C(O)XR⁶, or N⁺(R⁶)(R^{6′})(R^{6″});
X is NH or O;
R⁵ is H, C(O)OR⁶, C(O)NHR⁶, R⁶C(O), linear or branched C₁-C₆ alkyl, alkyl alcohol, alkyl amine (including primary, secondary, tertiary amine, or quaternary ammonium), or alkyl carboxylic acid; C₂-C₆ carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;
R⁶, R^{6′}, and R^{6″} are independently H, C₁-C₆ alkyl, alkyl alcohol, alkyl amine (including primary, secondary, tertiary amine, or quaternary ammonium) or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or a pharmaceutical salt;
or R² can be absent and C-9 directly links to L, and R², R³, X, C-10 and C-9 can join together to form a 5-, 6- or 7-member heterocyclic ring.

25. The conjugate according to claim 22, having Formula (IV): 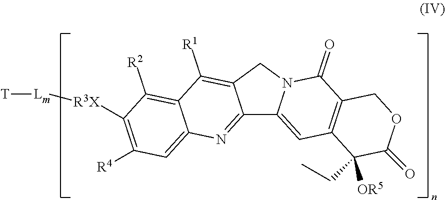
or a pharmaceutically acceptable salt, hydrate, or hydrated salt thereof; or a polymorphic crystalline structure thereof; or an isotope, optical isomer, racemate, diastereomes or enantiomer thereof;
wherein T is a targeting or binding ligand; L is a releasable linker; n is 1-30; and m is 1-10;
inside the bracket is a camptothecin analog wherein:
R¹ and R² are independently H, NR⁶R^{6′}, —N⁺R⁶R^{6′}R^{6″}, OH, SH, linear or branched C₁-C₆ alkyl, alkyloxyl, alkyl amino (including primary, secondary, tertiary amino, or quaternary ammonium), oxylcarbonyl, aminocarbonyl, aminoalkyl, oxylalkyl, aminoalkylamino, oxylalkylamino, aminoalkyloxyl, oxylalkyloxyl, or alkyl carboxylic; C₂-C₆ heteroalkyl, alkylcycloalkyl, heterocycloalkyl, heterocyclic, oxylcycloalkyl, aminocycloalkyl, heteroalkylcycloalkyl, alkylcarbonyl, aminoalkylcarbonyl, oxylalkylcarbonyl, alkyl ether, alkyl ester, alkyl amide, oxylalkylether, aminoalkylether, oxylalkylester, aminoalkylester, oxylalkylamide, aminoalkylamide, or oxime; NH₂, or OH;
R³ is independently —NHC(O)—, —C(O)—, SO₂—, —SO₂NH—, —NR⁶SO₂—, R⁶NHC(O), R⁶OC(O), SO₂R⁶, SO₃R⁶, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR^{6′})₂, R⁶C(O), C(O)N R⁶R^{6′}; linear or branched C₁-C₆ alkyl, alkyl alcohol, alkyl amine (including primary, secondary, tertiary amine), or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide, or oxime;
R⁴ is F, Cl, Br, I, CN, NO₂, SO₃H, OR⁶, SR⁶, S(O₂)R⁶, NH(R⁶)S(O₂)R^{6′}, N(R⁶)(R^{6′}), C(O)XR⁶, or N⁺(R⁶)(R^{6′})(R^{6″});
X is NH or O;
R⁵ is H, C(O)OR⁶, C(O)NHR⁶, R⁶C(O), linear or branched C₁-C₆ alkyl, alkyl alcohol, alkyl amine (including primary, secondary, tertiary amine, or quaternary ammonium), or alkyl carboxylic acid; C₂-C₆ carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;
R⁶, R^{6′}, and R^{6″} are independently H, C₁-C₆ alkyl, alkyl alcohol, alkyl amine (including primary, secondary, tertiary amine, or quaternary ammonium) or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or a pharmaceutical salt;
or R³ can be absent and X of C-10 directly links to L, and R², R³, X, C-10 and C-9 can join together to form a 5-, 6- or 7-member heterocyclic ring.

26. The conjugate according to claim 22, having Formula (V): 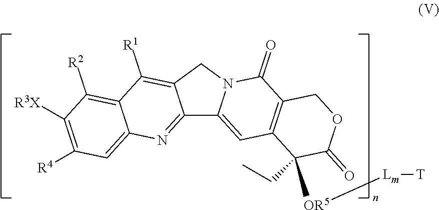
or a pharmaceutically acceptable salt, hydrate, or hydrated salt thereof; or a polymorphic crystalline structure thereof; or an isotope, optical isomer, racemate, diastereomer or enantiomer thereof;
wherein T is a targeting or binding ligand; L is a releasable linker; n is 1-30; and m is 1-10;
inside the bracket is a camptothecin analog, wherein:
R¹ and R² are independently H, NR⁶R^{6′}, —N⁺R⁶R^{6′}R^{6″}, OH, SH, linear or branched C₁-C₆ alkyl, alkyloxyl, alkyl amino (including primary, secondary, tertiary amino, or quaternary ammonium), oxylcarbonyl, aminocarbonyl, aminoalkyl, oxylalkyl, aminoalkylamino, oxylalkylamino, aminoalkyloxyl, oxylalkyloxyl, or alkyl carboxylic; C₂-C₆ heteroalkyl, alkylcycloalkyl, heterocycloalkyl, heterocyclic, oxylcycloalkyl, aminocycloalkyl, heteroalkylcycloalkyl, alkylcarbonyl, aminoalkylcarbonyl, oxylalkylcarbonyl, alkyl ether, alkyl ester, alkyl amide, oxylalkylether, aminoalkylether, oxylalkylester, aminoalkylester, oxylalkylamide, aminoalkylamide, or oxime; NH₂, or OH;
R³ is independently R⁶NHC(O)—, R⁶C(O)—, R⁶SO₂, —SO₂NHR⁶, R⁶OC(O), R^{6′}SO₂R⁶—, SO₃R⁶, PR⁶R^{6′}, POR⁶R^{6′}, CH₂OP(O)(OR⁶)₂, C(O)OP(O)(OR⁶)₂, PO(OR⁶)(OR^{6′}), P(O)(OR⁶)OP(O)(OR^{6′})₂, R⁶C(O), C(O)N R⁶R^{6′}; linear or branched C₁-C₆ alkyl, alkyl alcohol, alkyl amine (including primary, secondary, tertiary amine), or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide, or oxime;
R⁴ is F, Cl, Br, I, CN, NO₂, SO₃H, OR⁶, SR⁶, S(O₂)R⁶, NH(R⁶)S(O₂)R^{6′}, N(R⁶)(R^{6′}), C(O)XR⁶, or N⁺(R⁶)(R^{6′})(R^{6″});
X is NH or O;
R⁵ is C(O)O, C(O)NH, R⁶C(O), linear or branched C₁-C₆ alkyl, alkyl alcohol, alkyl amine (including primary, secondary, tertiary amine, or quaternary ammonium), or alkyl carboxylic acid; C₂-C₆ carbonate, carbamide, heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid;
R⁶, R^{6′}, and R^{6″} are independently H, C₁-C₆ alkyl, alkyl alcohol, alkyl amine (including primary, secondary, tertiary amine, or quaternary ammonium) or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or a pharmaceutical salt;
or R⁵ can be absent and O of C-20 directly links to L, and R², R³, X, C-10 and C-9 can join together to form a 5-, 6- or 7-member heterocyclic ring.

27. The conjugate according to claim 22, wherein camptothecin analog linked to linker L has one of structures of II-1 to II-61, III-1 to III-52, IV-1 to IV-47, and V-1 to V-61 below: 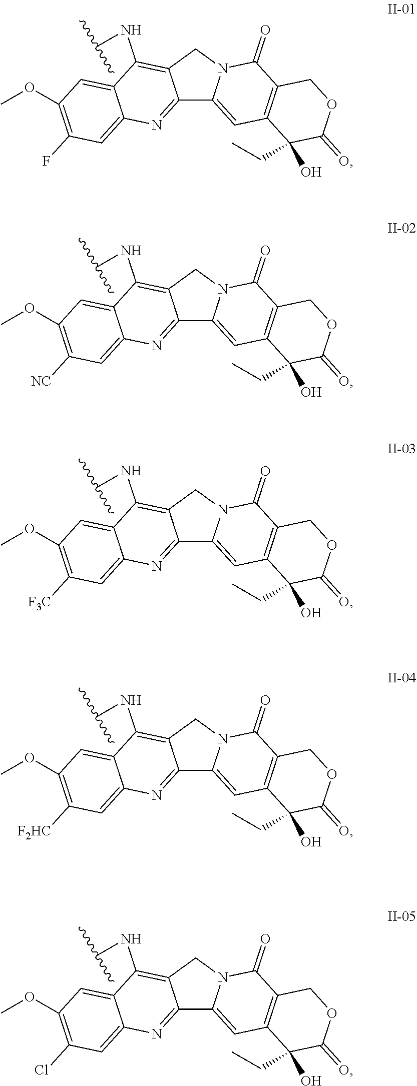 [SMILES: *Nc1c2c(nc3cc(F)c(OC)cc13)-c1cc3c(c(=O)n1C2)COC(=O)[C@]3(O)CC ; *Nc1c2c(nc3cc(C#N)c(OC)cc13)-c1cc3c(c(=O)n1C2)COC(=O)[C@]3(O)CC ; *Nc1c2c(nc3cc(C(F)(F)F)c(OC)cc13)-c1cc3c(c(=O)n1C2)COC(=O)[C@]3(O)CC ; *Nc1c2c(nc3cc(C(F)F)c(OC)cc13)-c1cc3c(c(=O)n1C2)COC(=O)[C@]3(O)CC ; *Nc1c2c(nc3cc(Cl)c(OC)cc13)-c1cc3c(c(=O)n1C2)COC(=O)[C@]3(O)CC] 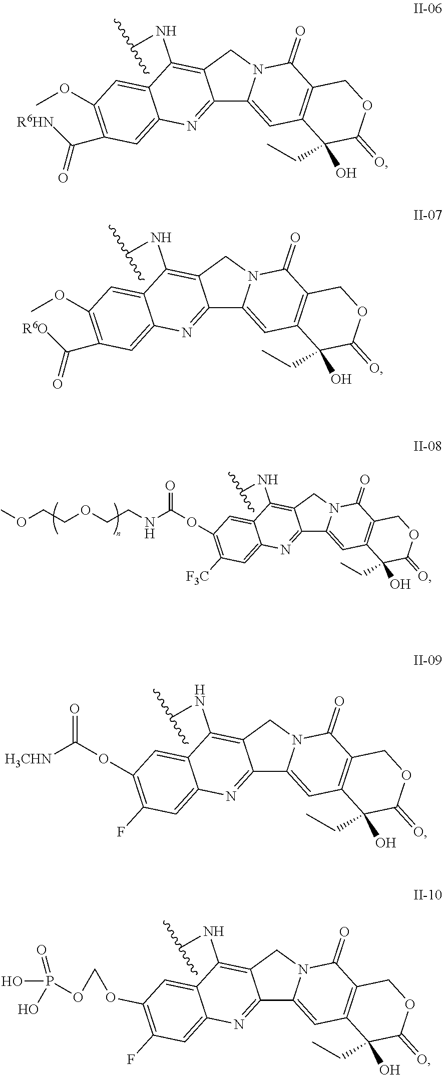 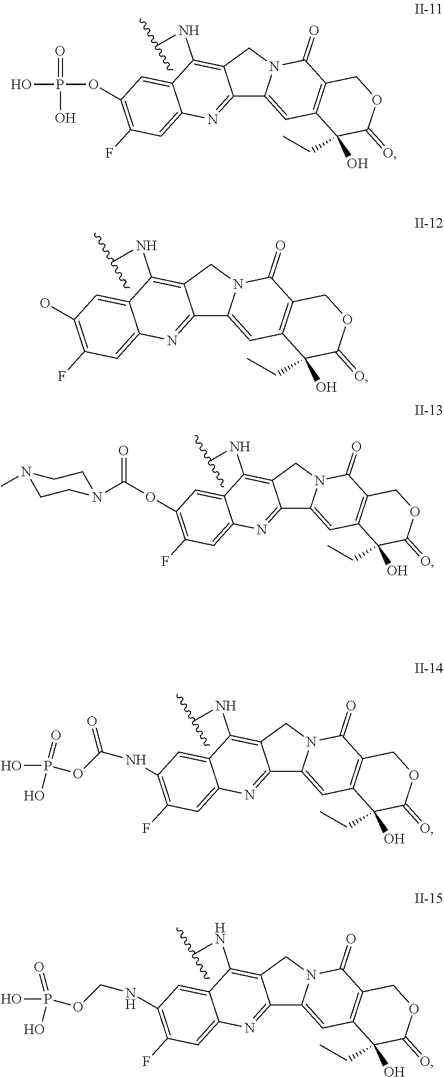 [SMILES: *Nc1c2c(nc3cc(F)c(OP(=O)(O)O)cc13)-c1cc3c(c(=O)n1C2)COC(=O)[C@]3(O)CC ; *Nc1c2c(nc3cc(F)c(O)cc13)-c1cc3c(c(=O)n1C2)COC(=O)[C@]3(O)CC ; *Nc1c2c(nc3cc(F)c(OC(=O)N4CCN(C)CC4)cc13)-c1cc3c(c(=O)n1C2)COC(=O)[C@]3(O)CC ; *Nc1c2c(nc3cc(F)c(NC(=O)OP(=O)(O)O)cc13)-c1cc3c(c(=O)n1C2)COC(=O)[C@]3(O)CC ; *Nc1c2c(nc3cc(F)c(NCOP(=O)(O)O)cc13)-c1cc3c(c(=O)n1C2)COC(=O)[C@]3(O)CC] 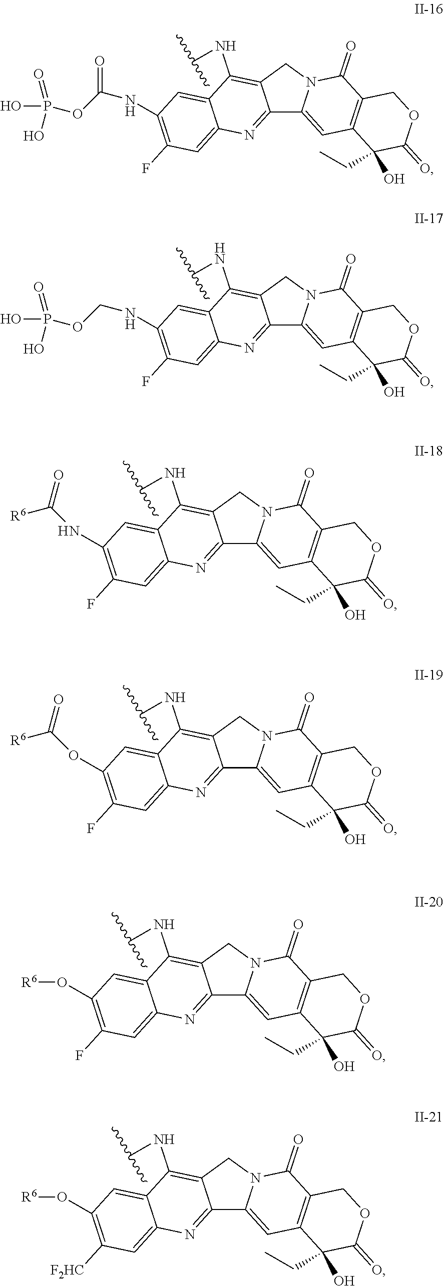 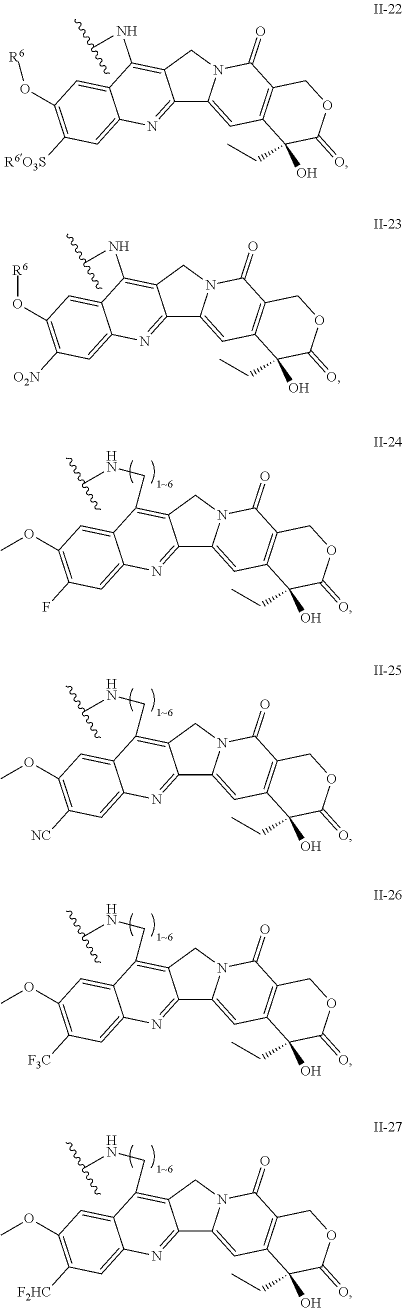 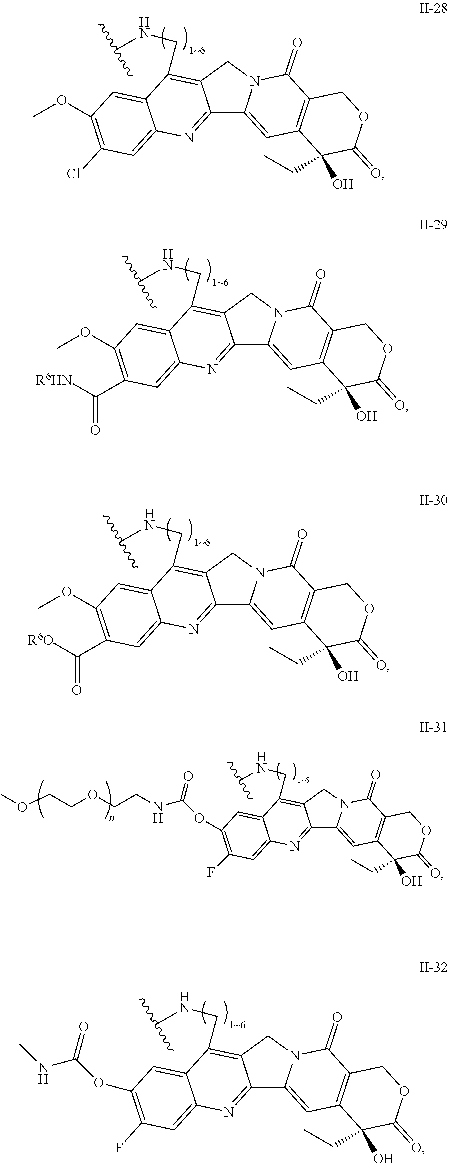 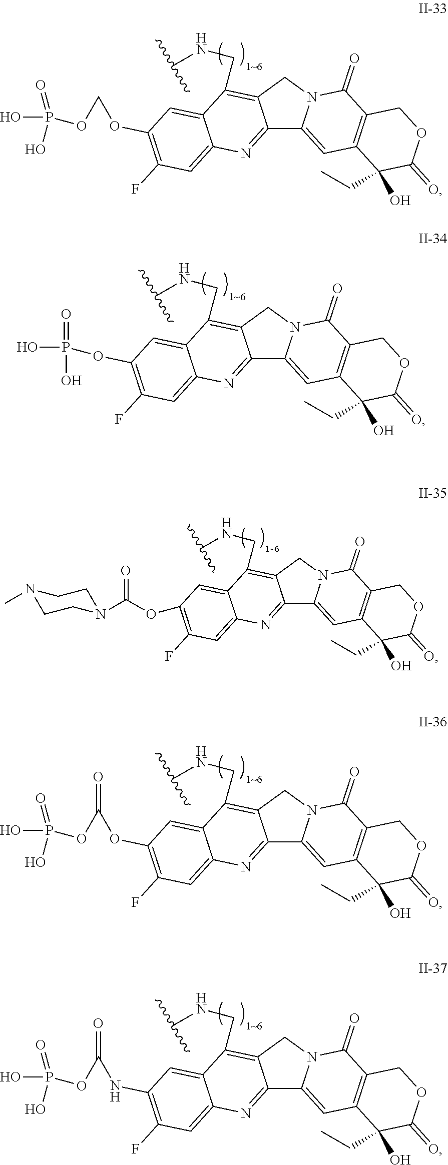 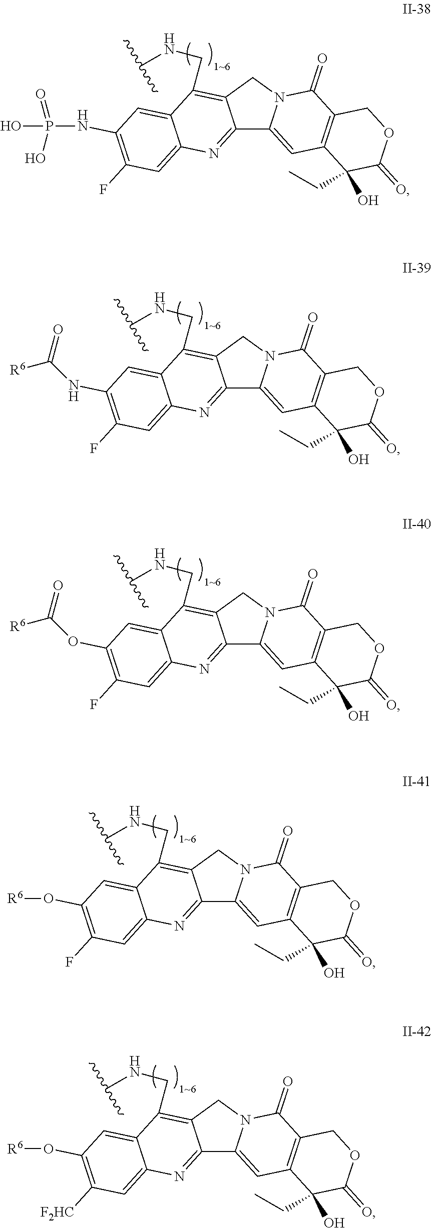 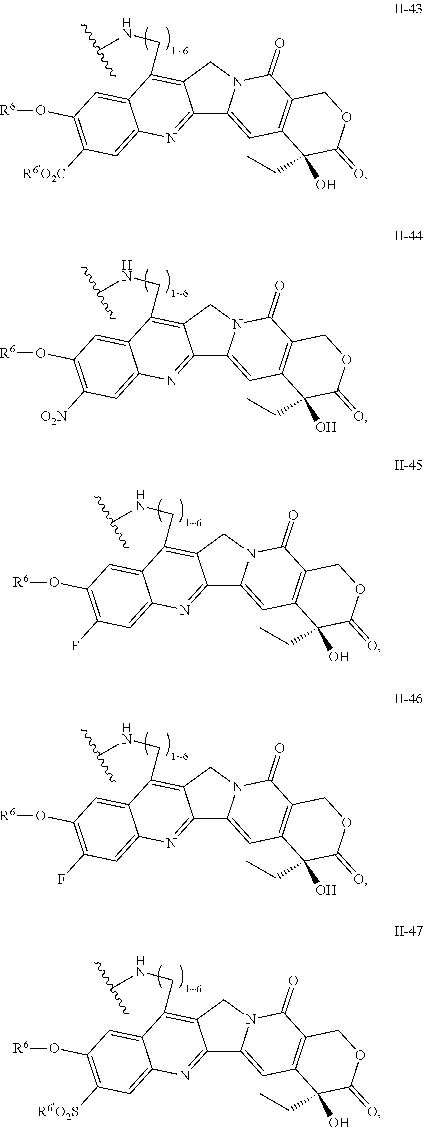 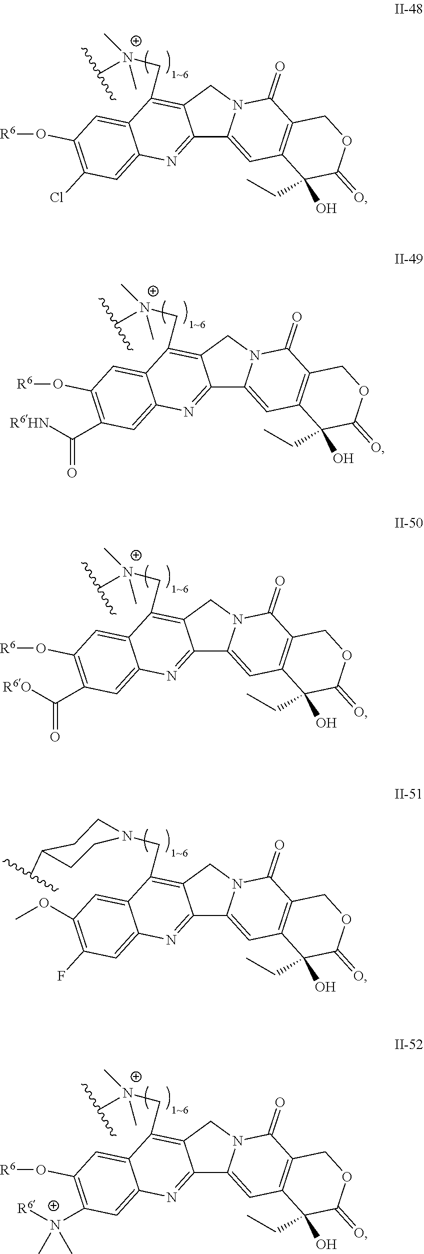 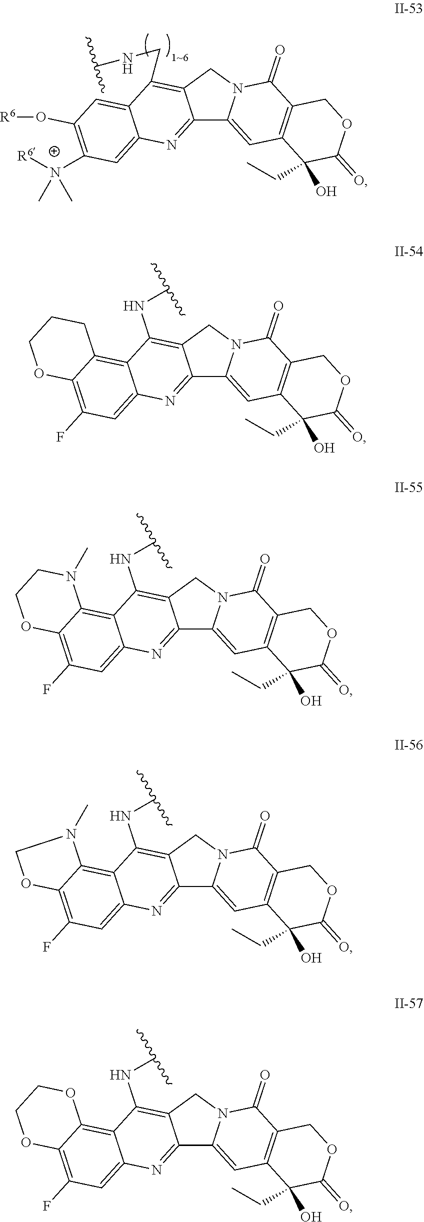 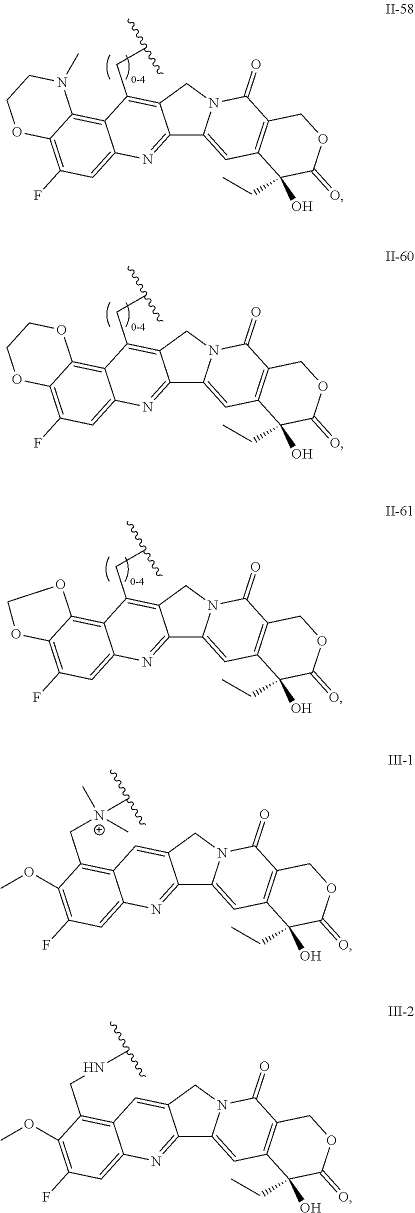 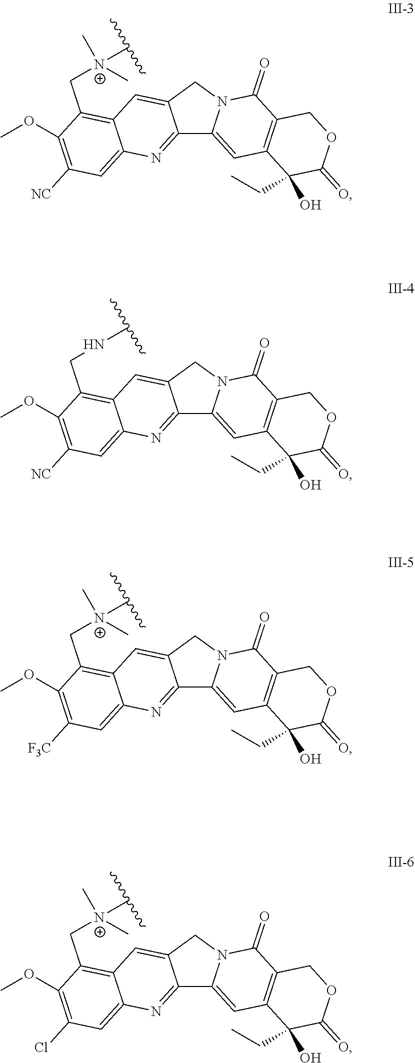 [SMILES: *[N+](C)(C)Cc1c(OC)c(C#N)cc2nc3c(cc12)Cn1c-3cc2c(c1=O)COC(=O)[C@]2(O)CC ; *NCc1c(OC)c(C#N)cc2nc3c(cc12)Cn1c-3cc2c(c1=O)COC(=O)[C@]2(O)CC ; *[N+](C)(C)Cc1c(OC)c(C(F)(F)F)cc2nc3c(cc12)Cn1c-3cc2c(c1=O)COC(=O)[C@]2(O)CC ; *[N+](C)(C)Cc1c(OC)c(Cl)cc2nc3c(cc12)Cn1c-3cc2c(c1=O)COC(=O)[C@]2(O)CC] 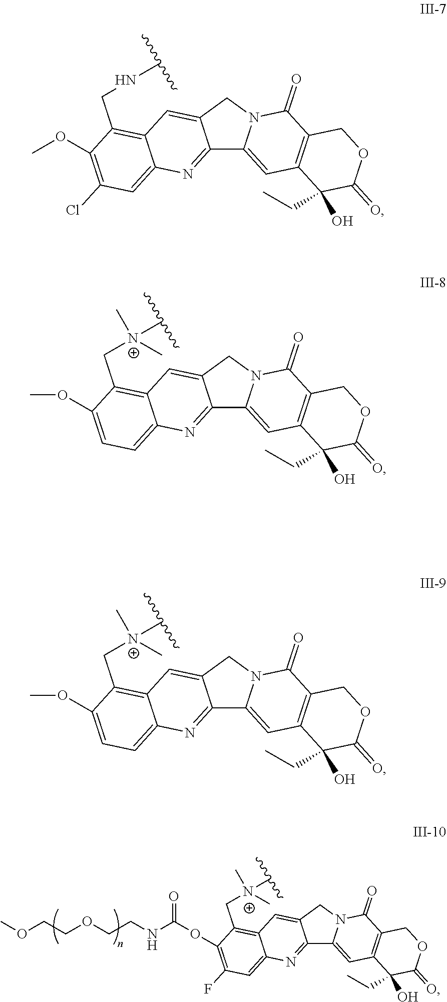 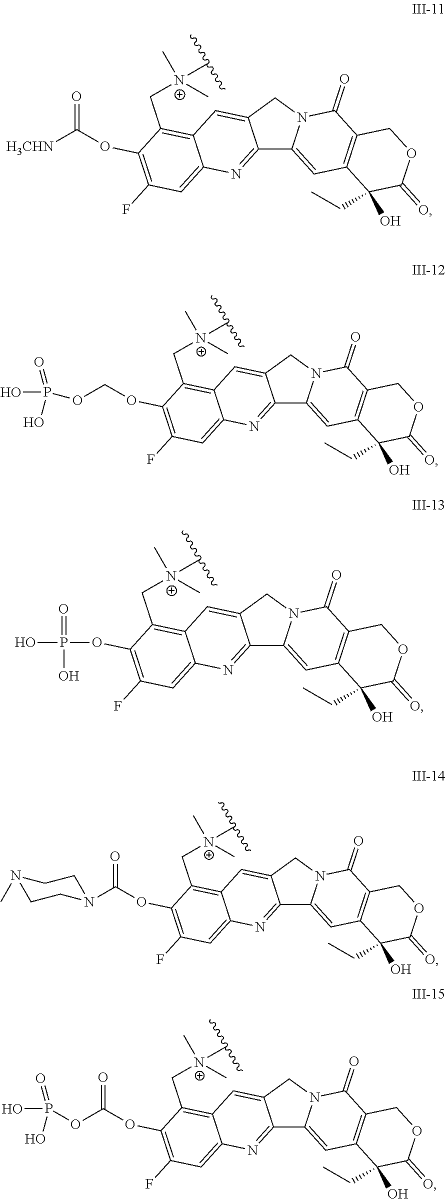 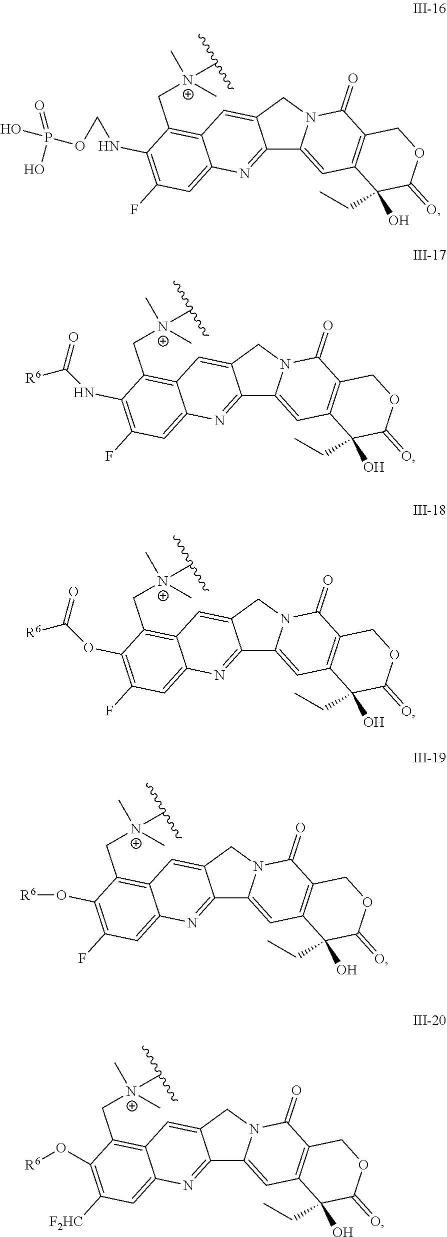 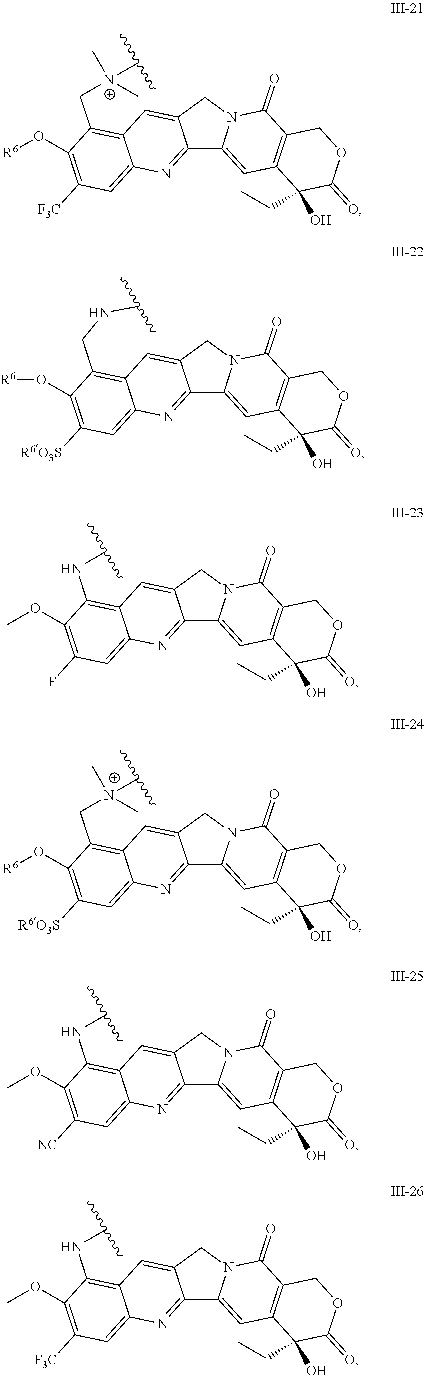 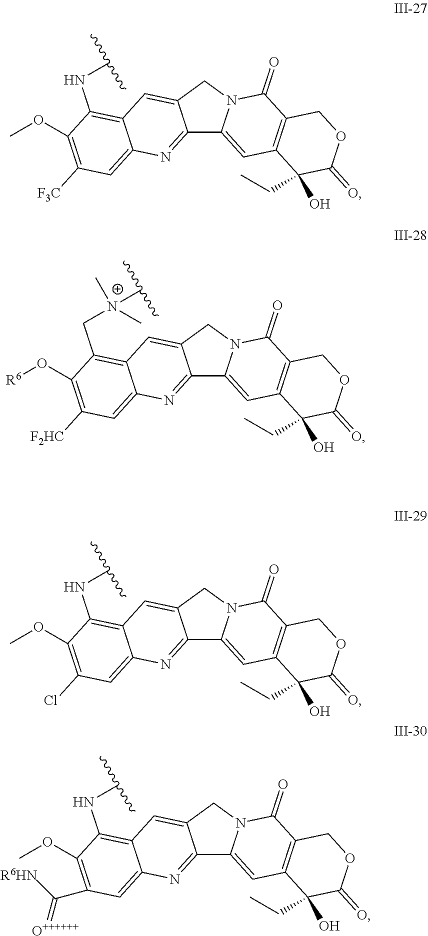 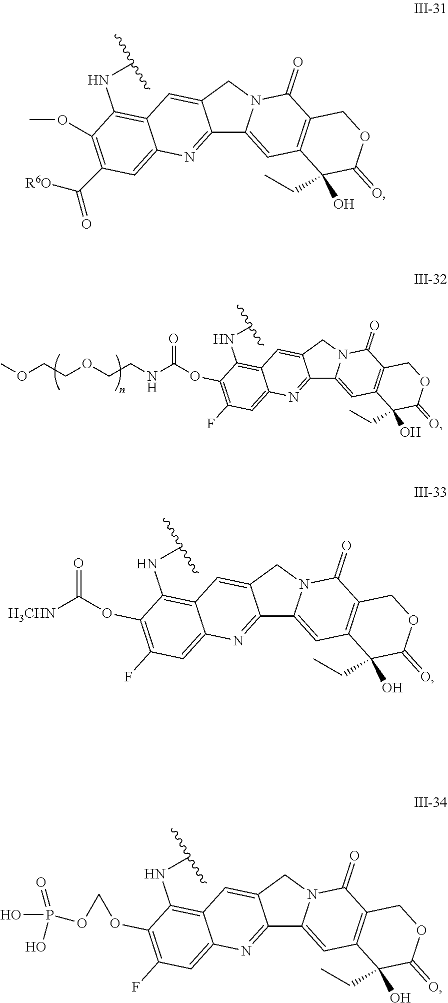 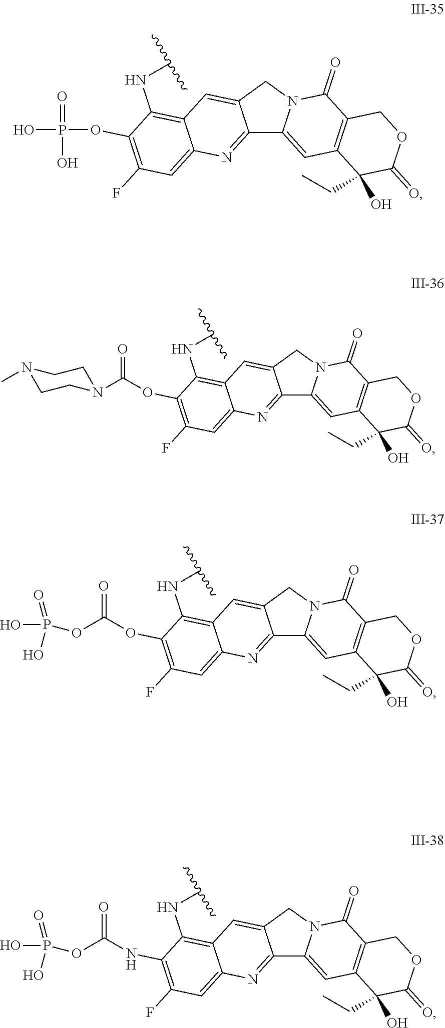 [SMILES: *Nc1c(OP(=O)(O)O)c(F)cc2nc3c(cc12)Cn1c-3cc2c(c1=O)COC(=O)[C@]2(O)CC ; *Nc1c(OC(=O)N2CCN(C)CC2)c(F)cc2nc3c(cc12)Cn1c-3cc2c(c1=O)COC(=O)[C@]2(O)CC ; *Nc1c(OC(=O)OP(=O)(O)O)c(F)cc2nc3c(cc12)Cn1c-3cc2c(c1=O)COC(=O)[C@]2(O)CC ; *Nc1c(NC(=O)OP(=O)(O)O)c(F)cc2nc3c(cc12)Cn1c-3cc2c(c1=O)COC(=O)[C@]2(O)CC] 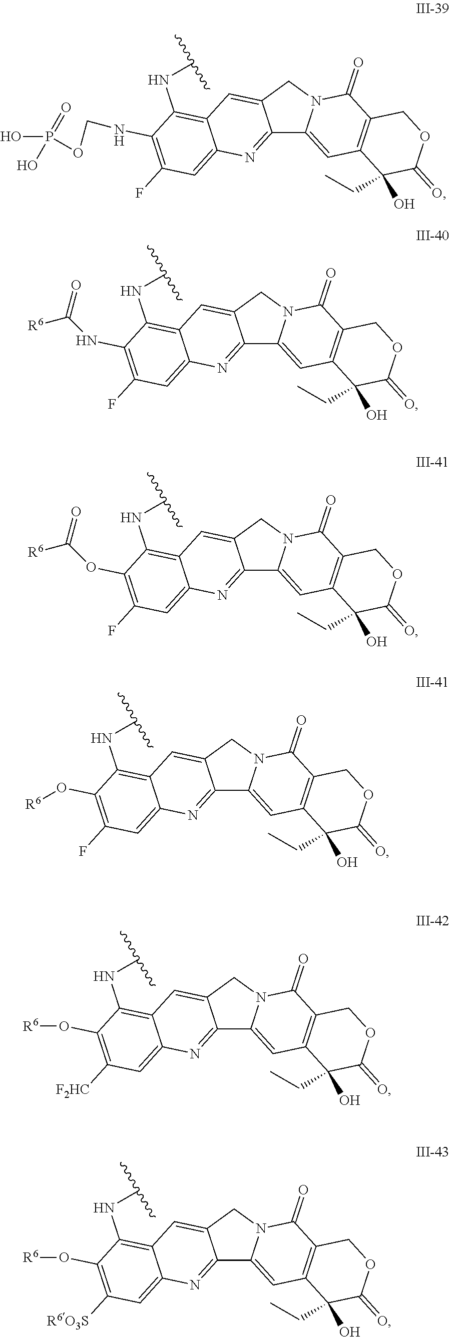 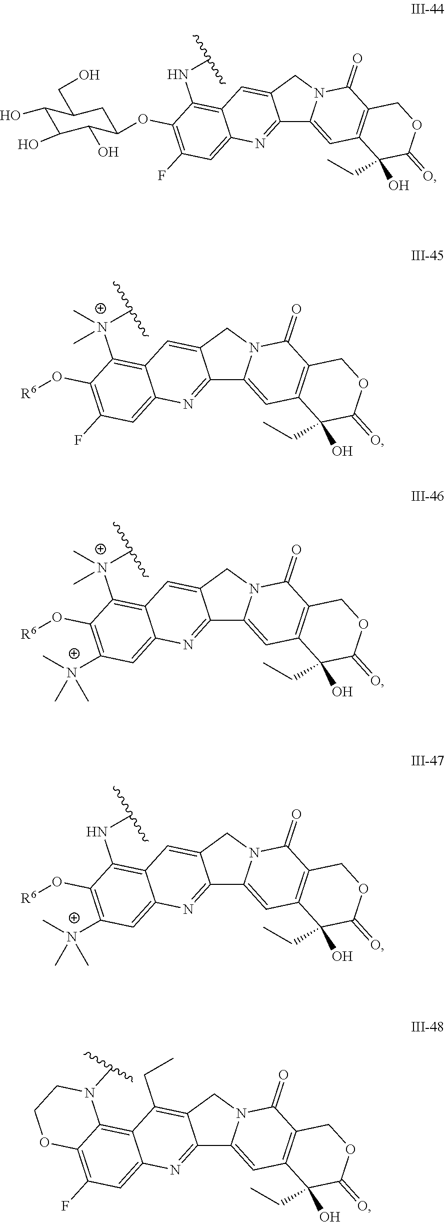 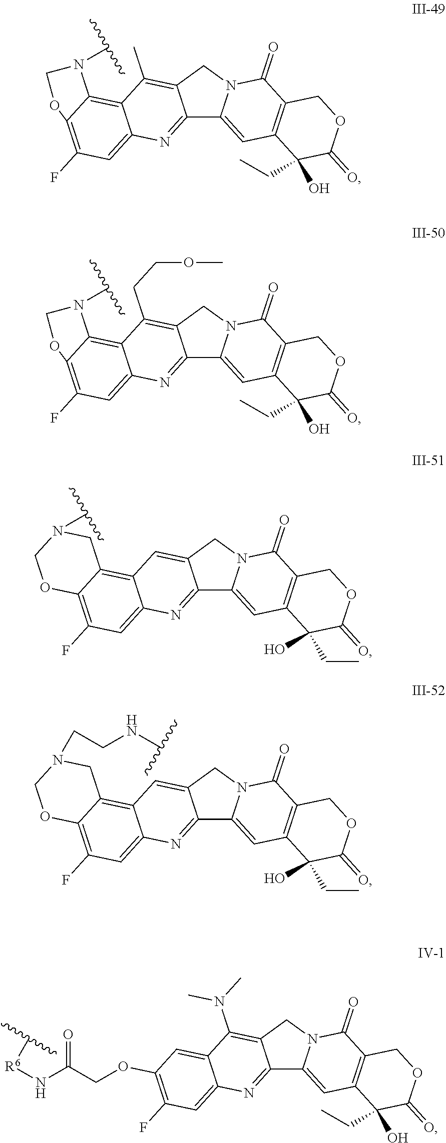 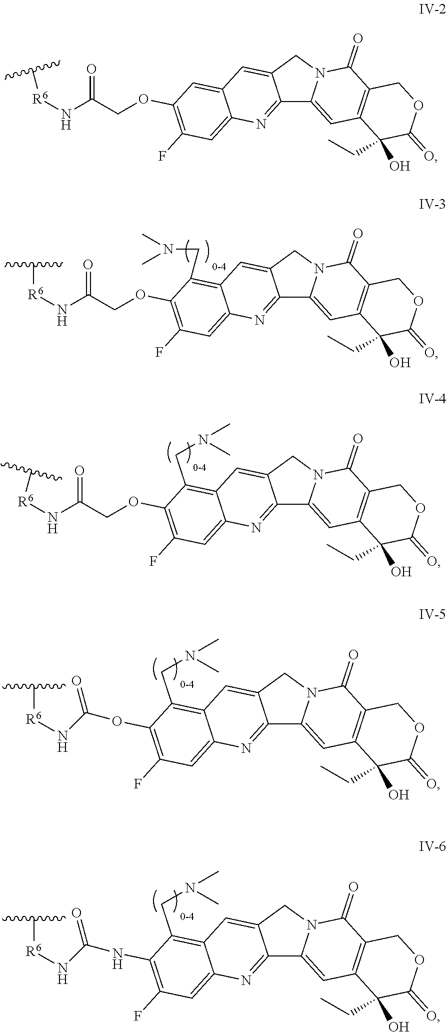 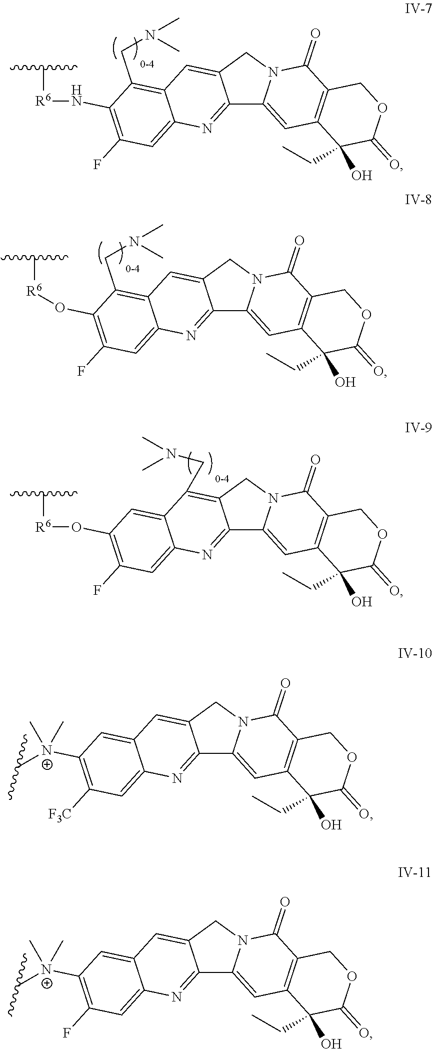 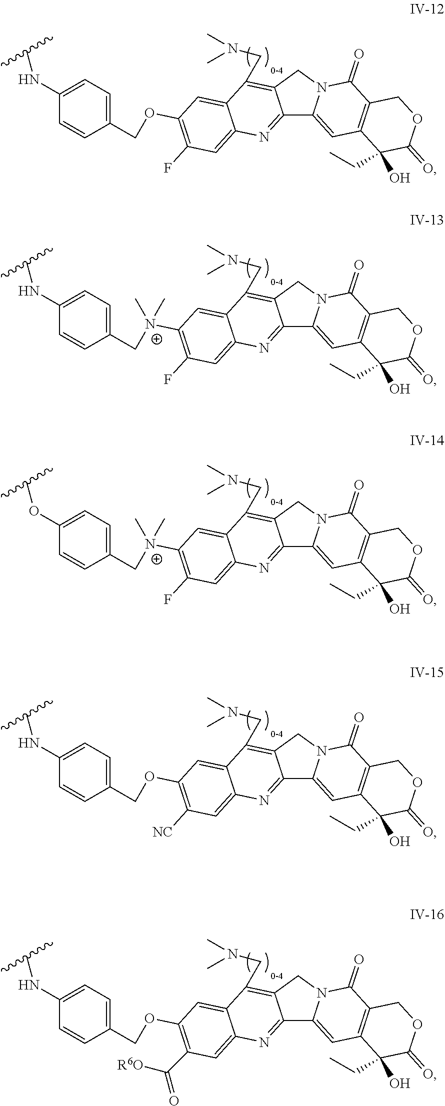 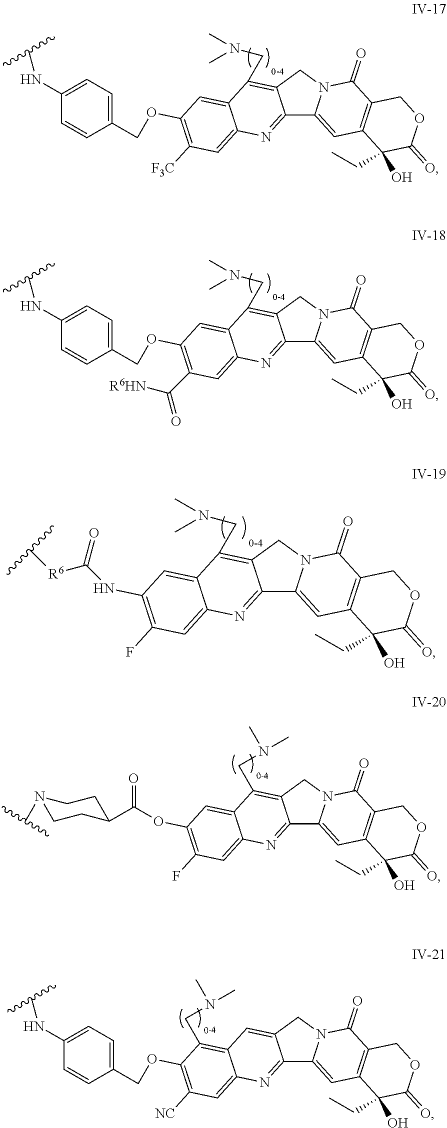 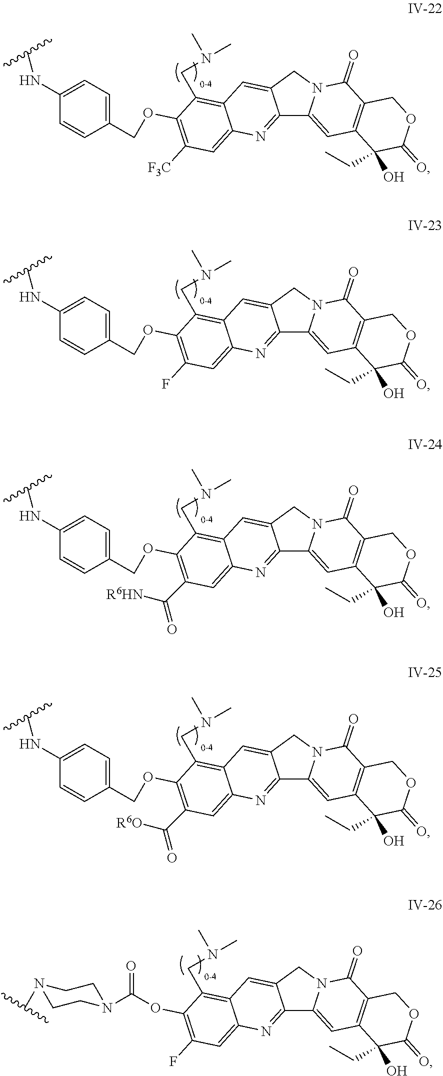 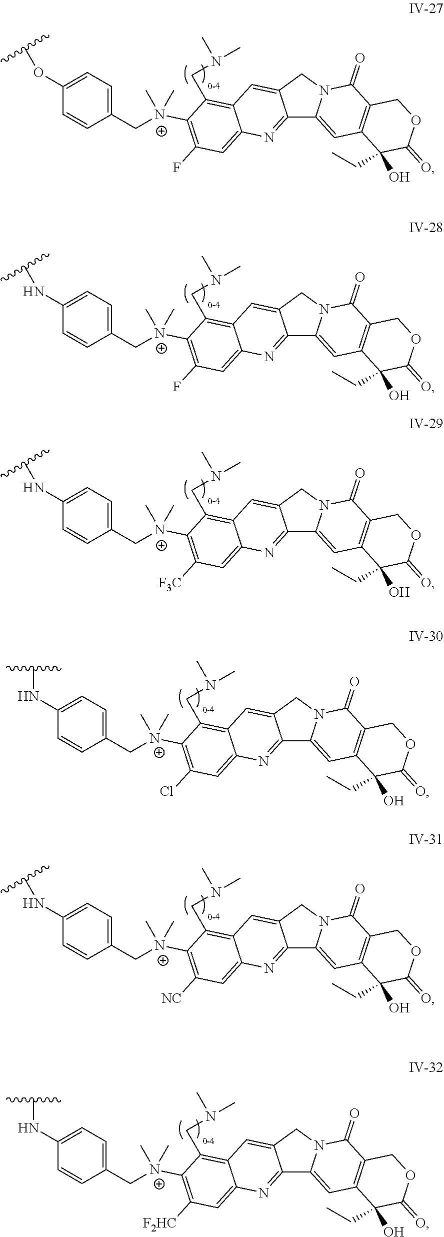 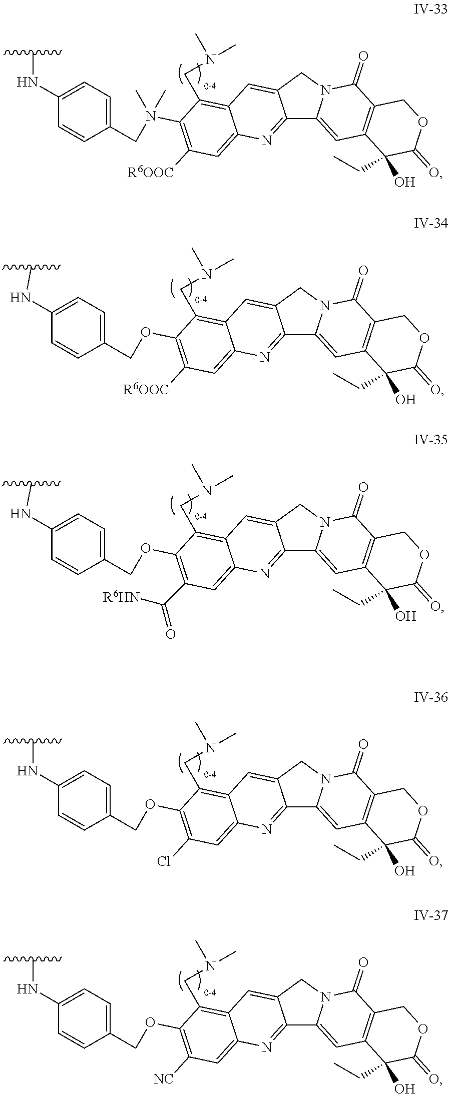 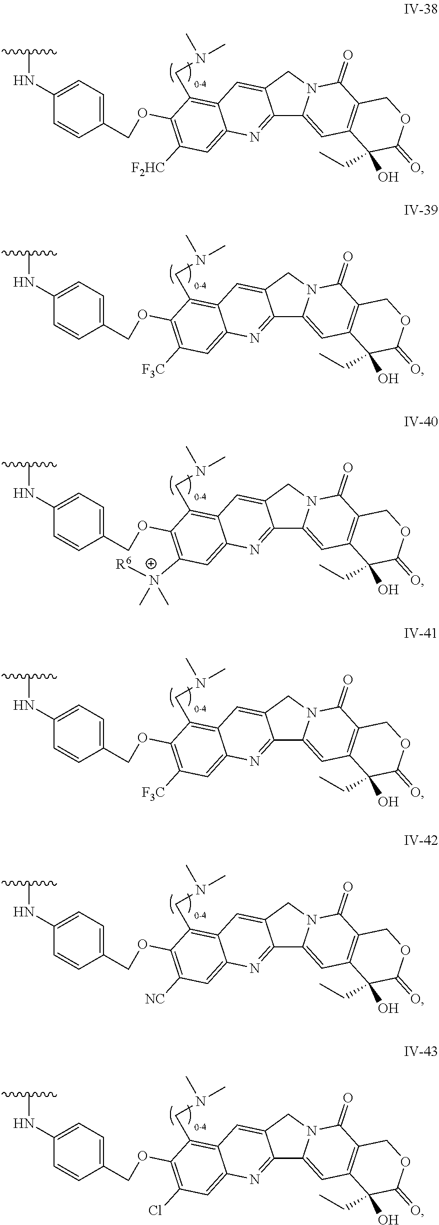 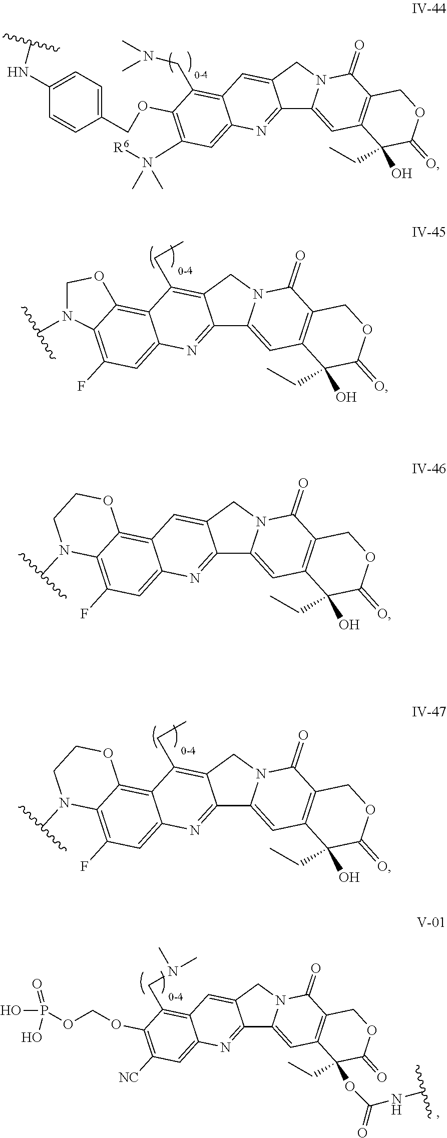 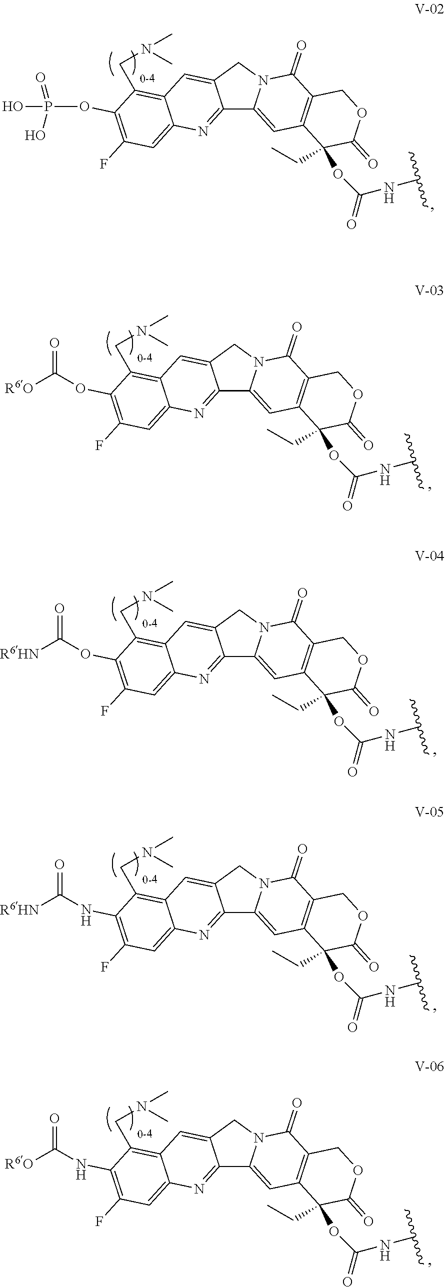 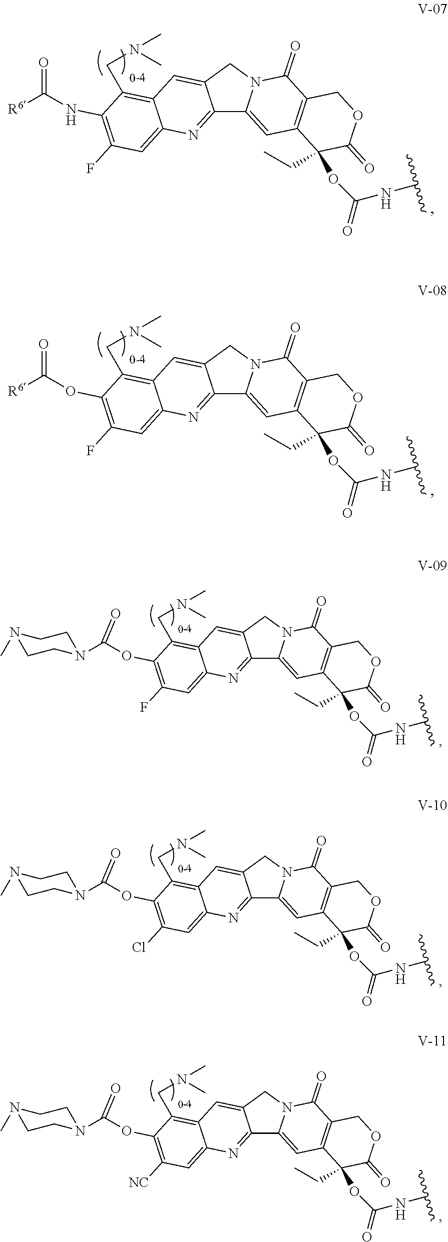 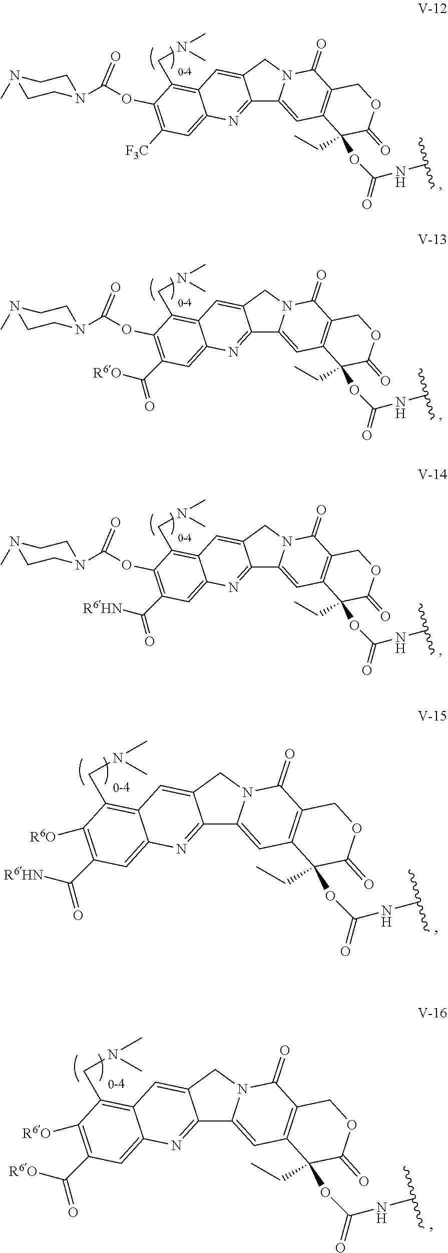 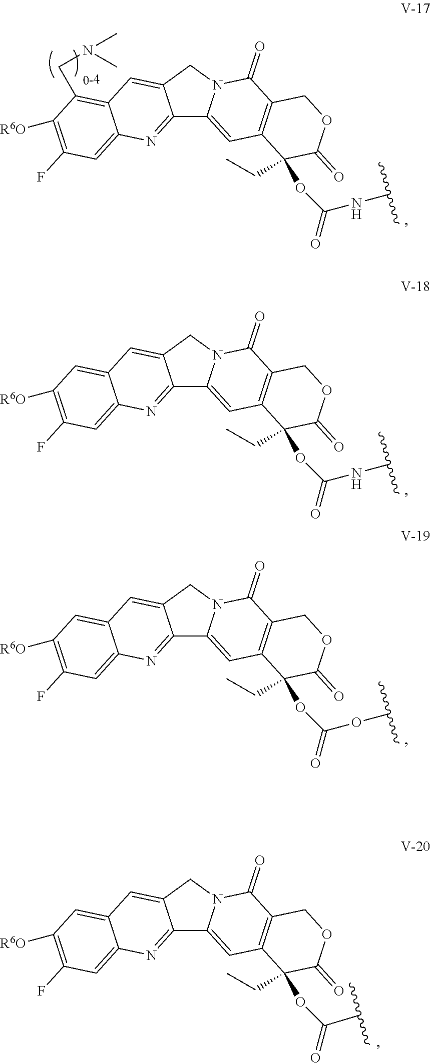 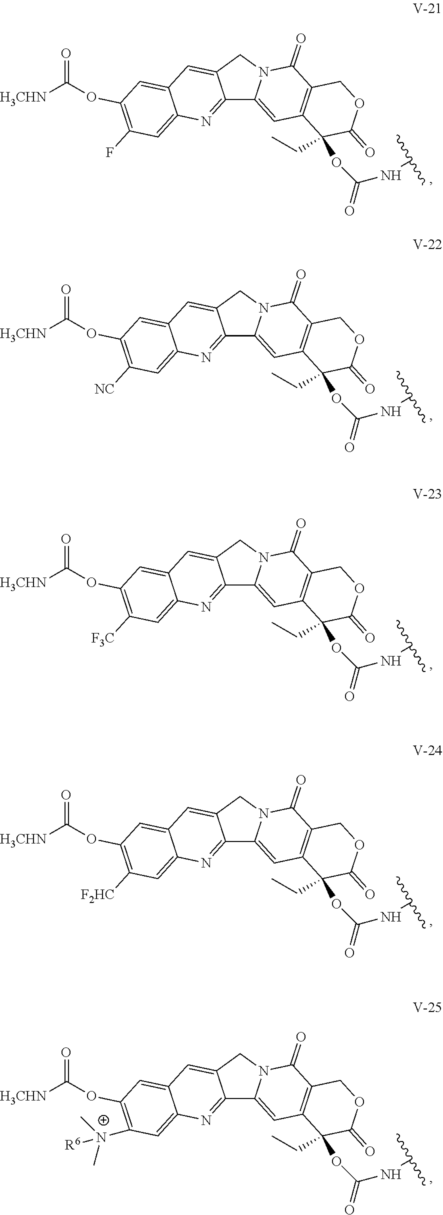 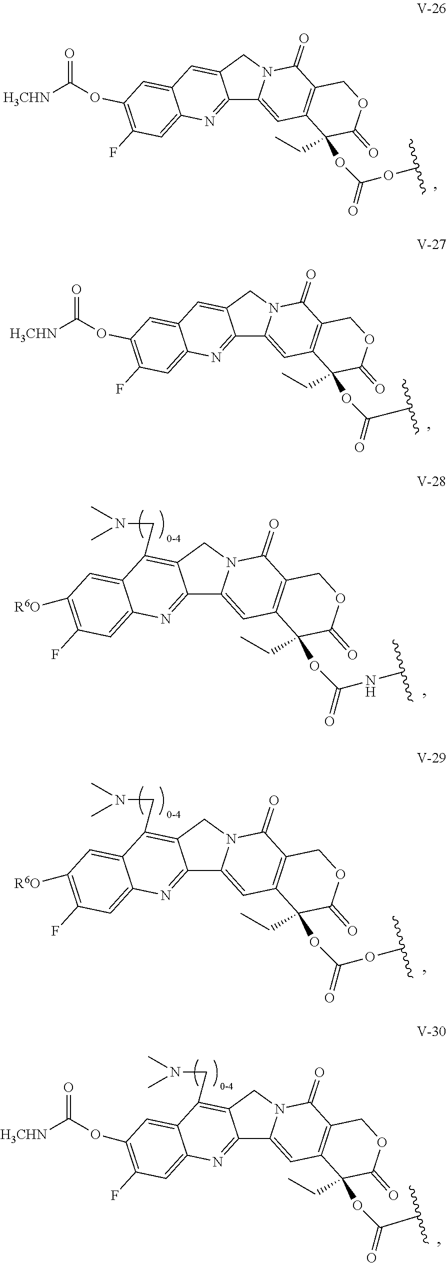 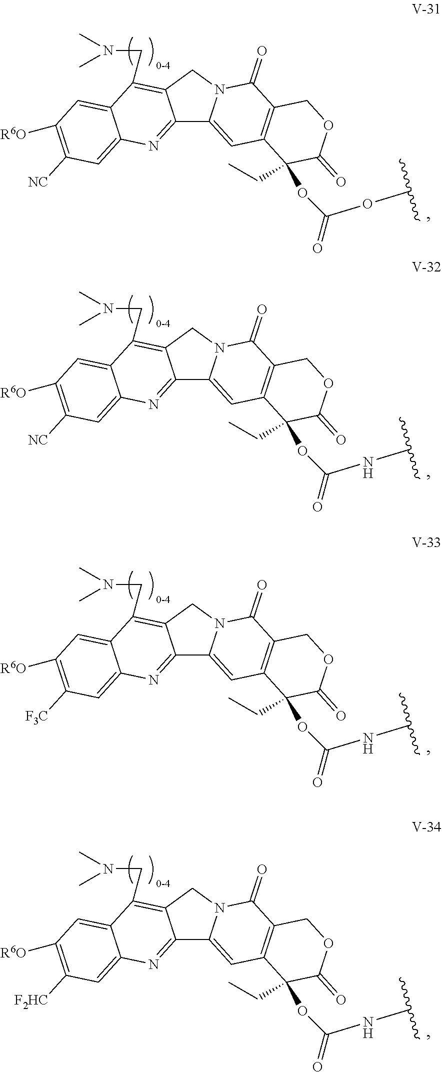 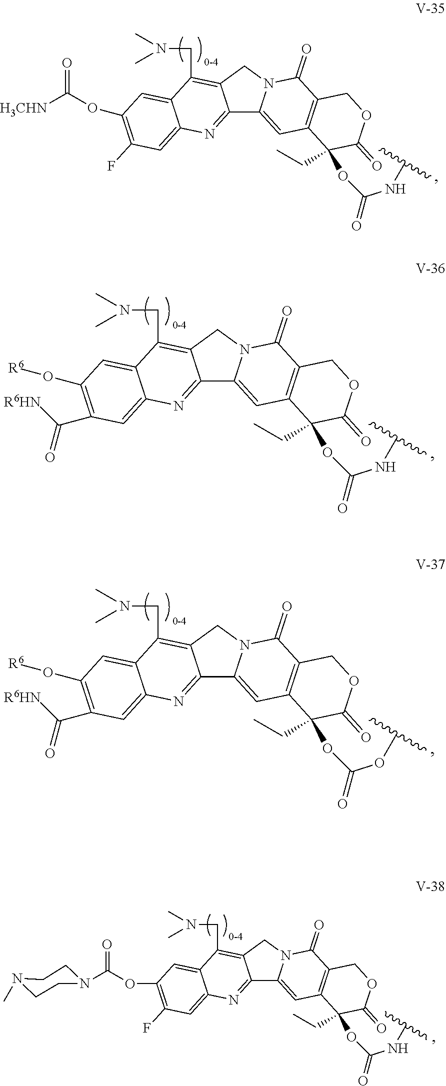 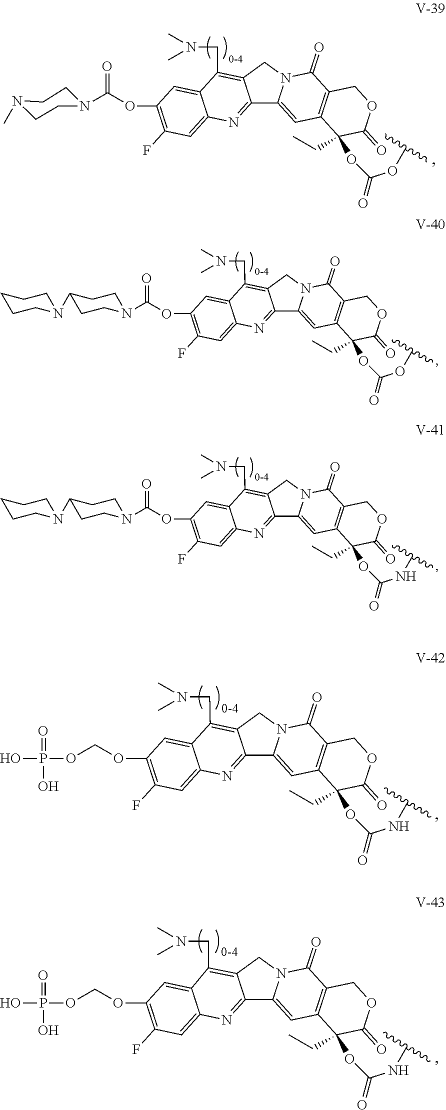 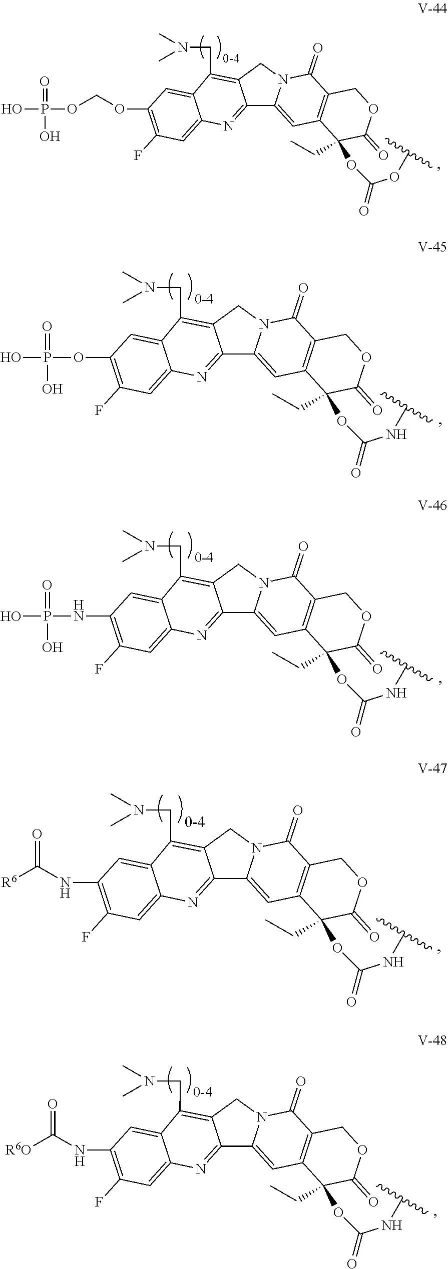 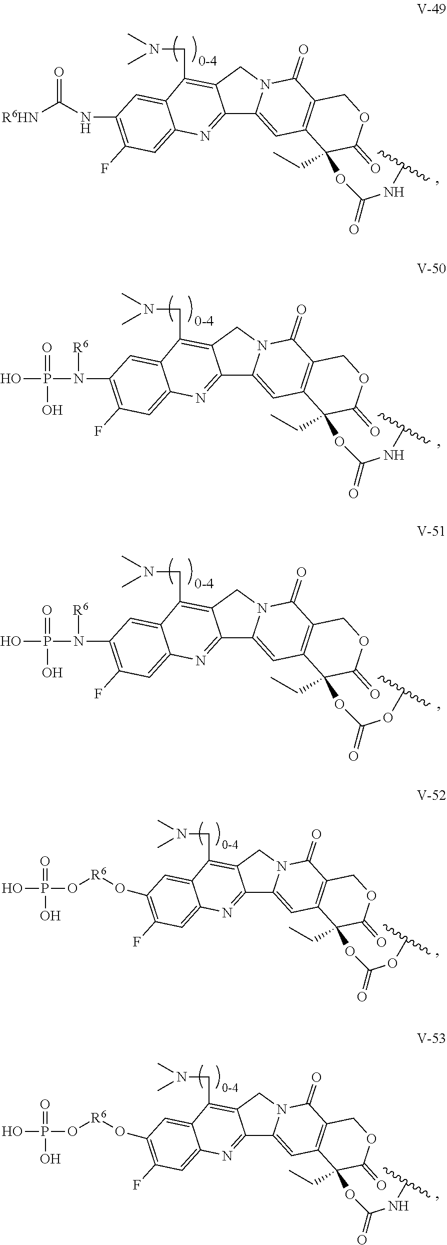 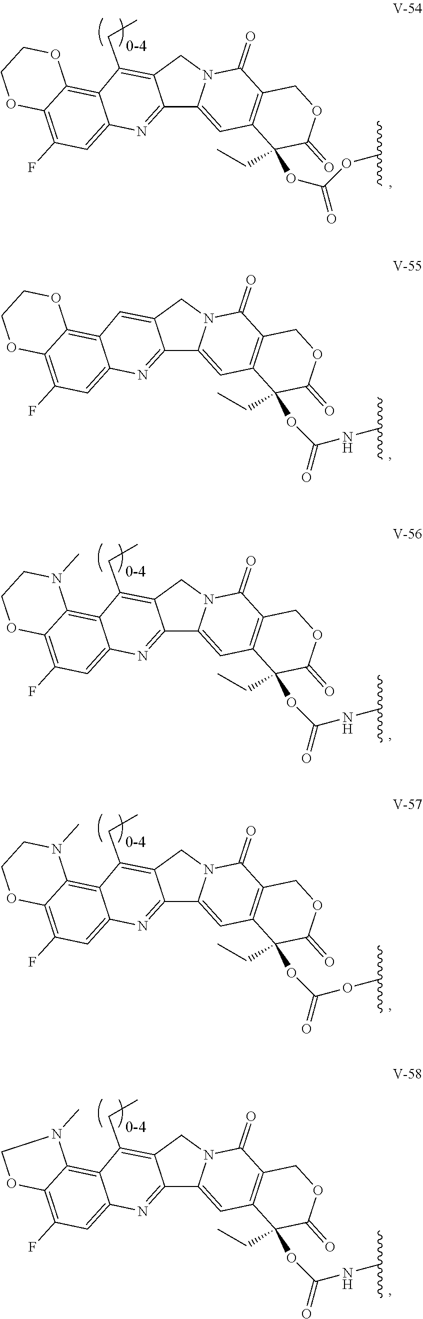 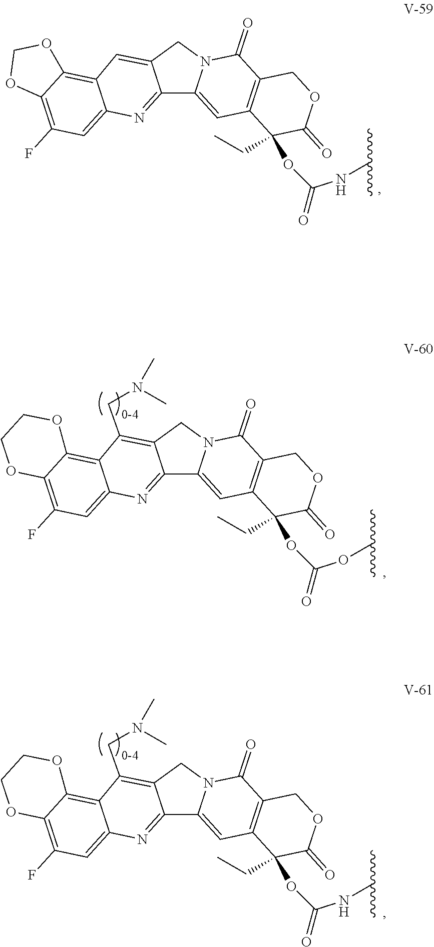
or a pharmaceutically acceptable salt, hydrate, or hydrated salt thereof; or a polymorphic crystalline structure thereof; or an isotope, optical isomer, racemate, diastereomer or enantiomer thereof;
wherein “ [symbol] ” is the site linked to linker L;
wherein R⁶, and R^{6′} are independently H, C₁-C₆ alkyl, alkyl alcohol, alkyl amine (including primary, secondary, tertiary amine, or quaternary ammonium) or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or a pharmaceutical salt.

28. The conjugate according to claim 22, wherein the linker L has an amino, sulfonamide, phosphamide or amino acid group which is linked to a side chain of formula (I-q): 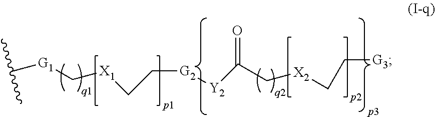
wherein [symbol] is a site linked to the sulfonyl, phosphate, amino, or carbonyl group of the linker; G, is NH, NHNH, C(═O), NHNHC(O), C(═O)NH, C(═NH)NH, CH₂, CH₂C(O), C(O)O, NHC(O)NH, or (Aa)ᵣ, (r=1-12); G₂ is NH, NHNH, C(═O), NHNHC(O), C(═O)NH, C(═NH)NH, CH₂, C(O)O, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)ᵣ, (r=1-12); X, and X₂ are independently O, CH₂, S, NH, N(R₁₂), *NH(R₁₂), *N(R₁₂)(R₁₃), C(O), OC(O), OC(O)O, NHSO₂NH, NHP(O)(NH)₂, SO₂NH, P(O)(NH)₂, NHS(O)NH, NHP(O)(OH)(NH), OC(O)NH, or NHC(O)NH; Y₂ is O, NH, NR₁, CH₂, S, or Ar; G₃ is OH, SH, OR₁, SR₁, OC(O)R₁, NHC(O)R₁₂, C(O)R₁₂, CH₃, NH₂, NR₁₂, +NH(R₁₂), *N(R₁₂)(R₁₃), C(O)OH, C(O)NH₂, NHC(O)NH₂, BH₂, BR₁₂R₁₃, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}—C(O)OH, OC(O)NH—(CH₂)_{q1}C(O)OH, NHCO(CH₂)_{q1}P(O)(OH)₂, NHC(O)(NH)(CH₂)_{q1}C(O)OH, CONH(CH₂)_{q1}—P(O)(OH)₂, NHS(O)₂(CH₂)_{q}, C(O)OH, CO(CH₂)_{q1}S(O)₂(OH), NHS(O)₂NH—(CH₂)_{q1}C(O)OH, OS(O)₂NH(CH₂)_{q1}C(O)OH, NHCO(CH₂)_{q1}S(O)₂(OH), NHP(O)(OH)(NH)—(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁, Ar, ArR₁₂, ArOH, ArNH₂, ArSH, ArNHR₁₂, or (Aa)_{q1}; p₁, p₂ and p₃ are independently 0-30 but are not 0 at the same time; q₁ and q₂ are independently 0-24.

29. The conjugate according to claim 28, wherein the side chain linker of the formula (I-q) is selected from: 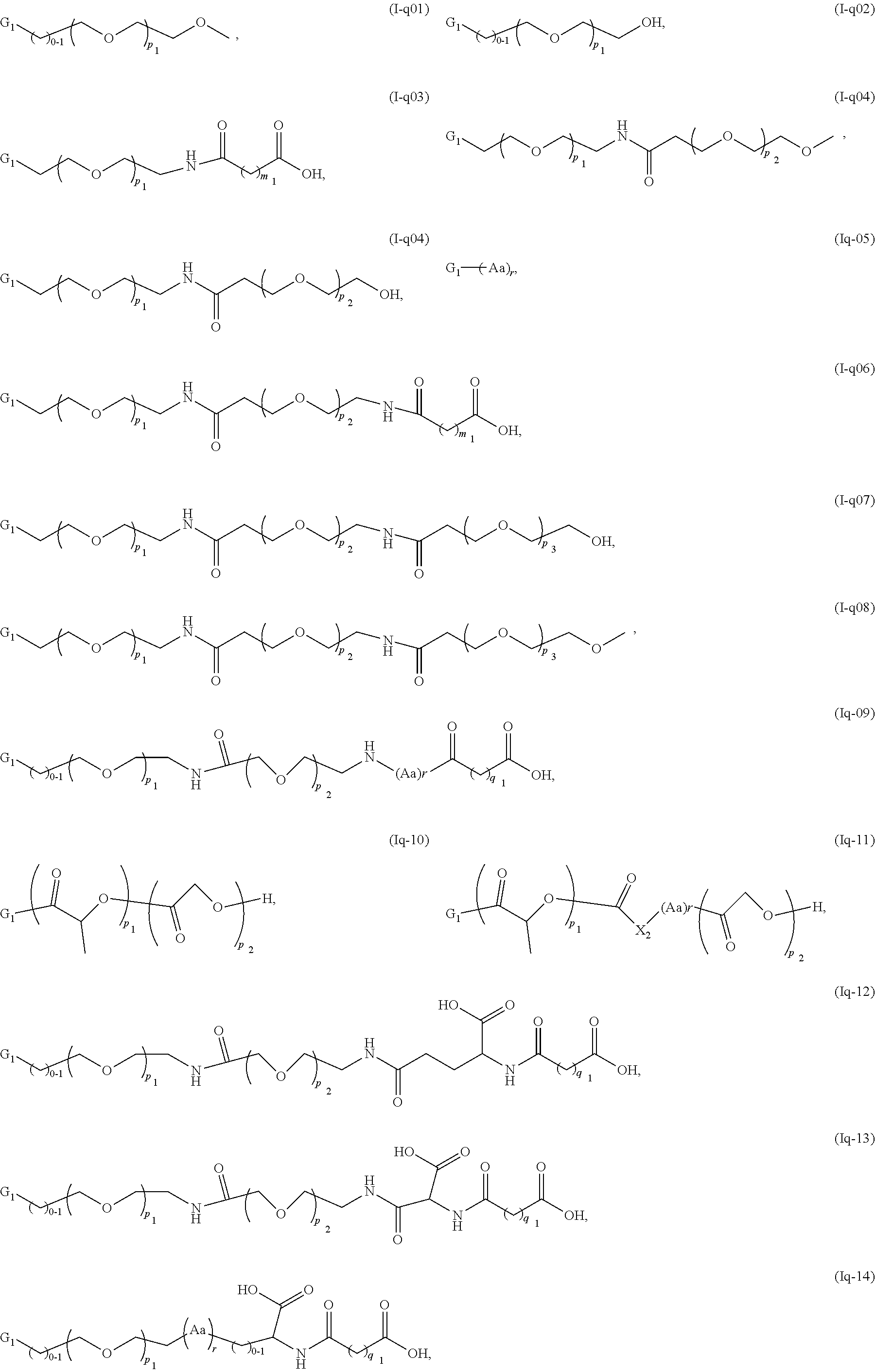
wherein G₁, p₁, p₂, p₃, Aa, r, X₂, q₁, m₁ are defined the same as in claim 28.

30. A process for preparing the conjugate of Formula (I), according to claim 22, comprising coupling a cell-binding molecule T with a conjugatable compound of Formula (VI): 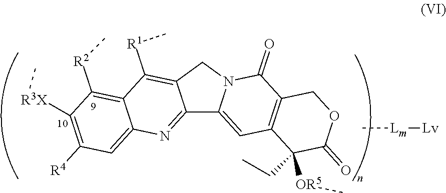
wherein R₁, R₂, R₃, R₄, R₅, L, X and m are defined the same as in claim 22;
Lv is a reacting group that can react with a thiol, amine, carboxylic acid, selenol, phenol or hydroxyl group on the cell-binding molecule.

31. The conjugate according to claim 22, having formula (IIq-1), (IIq-2), (IIq-3), (IIq-4), (IIq-5), (IIq-6), (IIq-7), or (IIq-8) below: 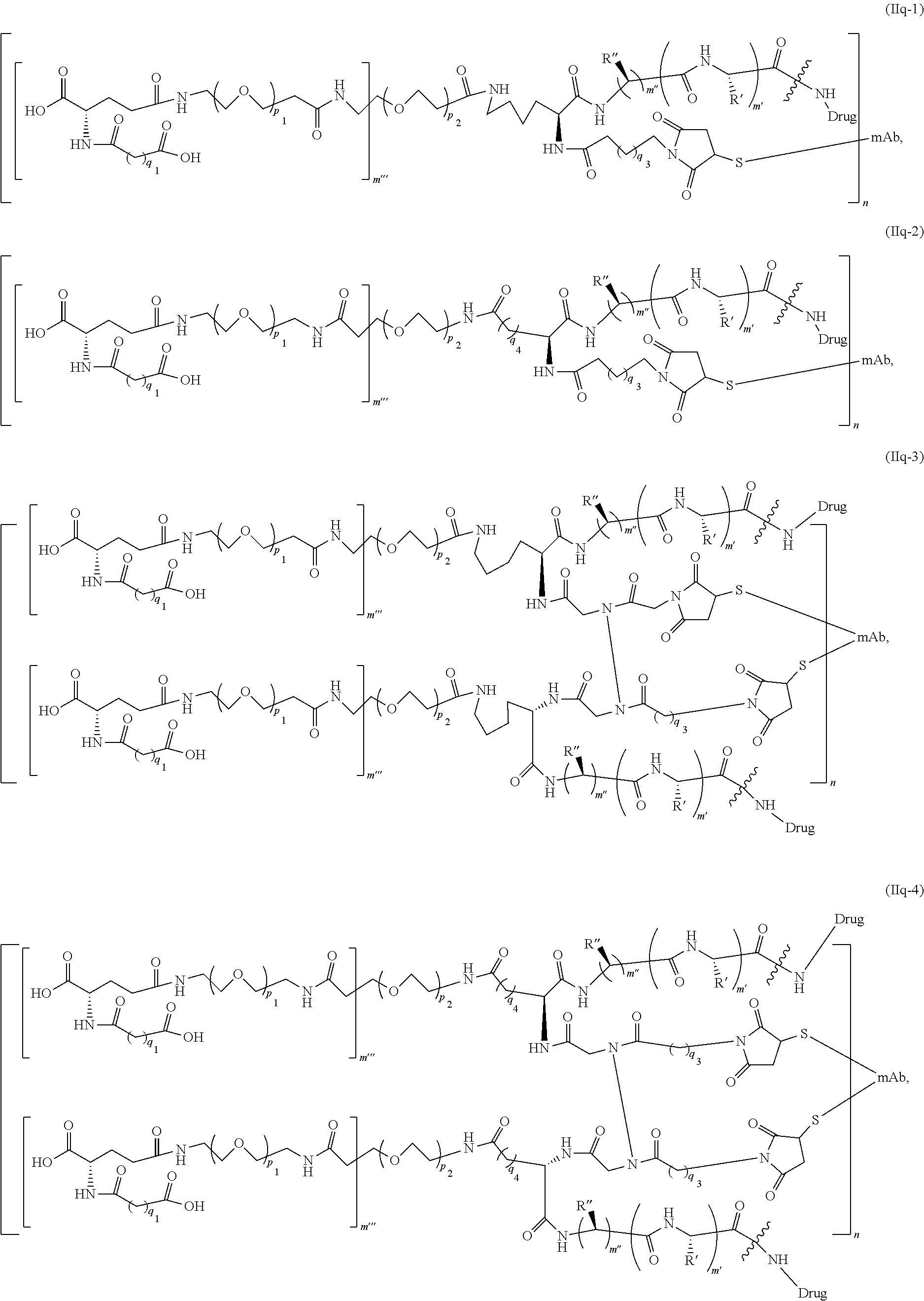 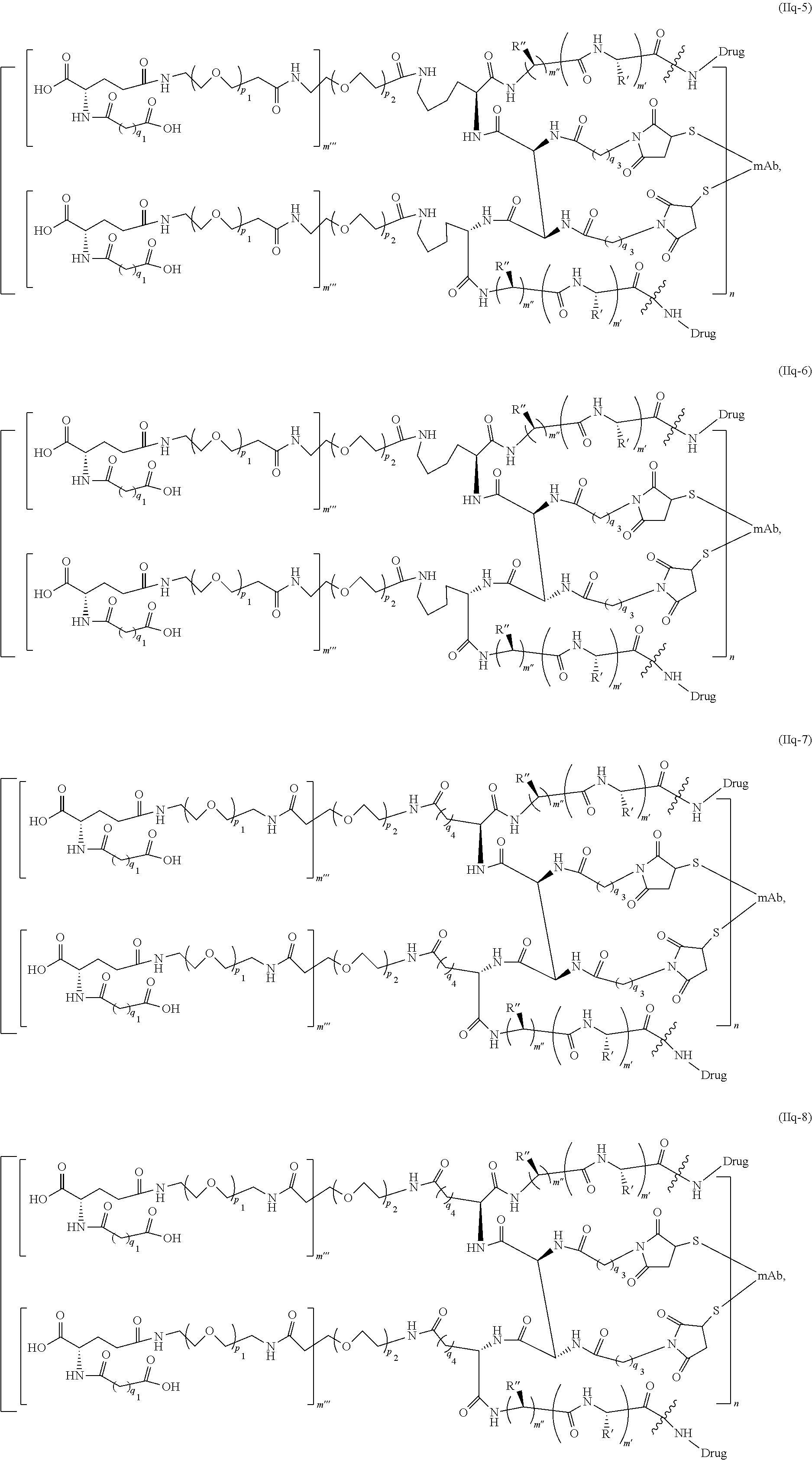
wherein R′ and R″ are independently H, Me, Et, ⁱPr, ⁱBu, Bz (CH₂C₆H₅), CH₂COOH, CH₂CH₂COOH, CH₂CONH₂, CH₂CH₂CONH₂, CH₂CH₂CH₂CH₂NH₂, CH₂CH₂SCH₃, CH₂OH, CH₂CH₂CH₂NHC(═NH)NH₂, CH(OH)CH₃, CH₂C₆H₄OH, or CH₂C₃N₂H₃; p₁ and p₂ are independently 0-24; q₁ is 1-18; q₃ is 0-6; q₄ is 0-4; m′ and m″ are independently 0-6; m″′ is 0 or 1; and mAb is a cell-binding molecule; NH-Drug has one of formulae II-1 to II-61, III-1 to III-52, IV-1 to IV-47, and V-1 to V-61 below; and [symbol] is a site linked to NH-Drug: 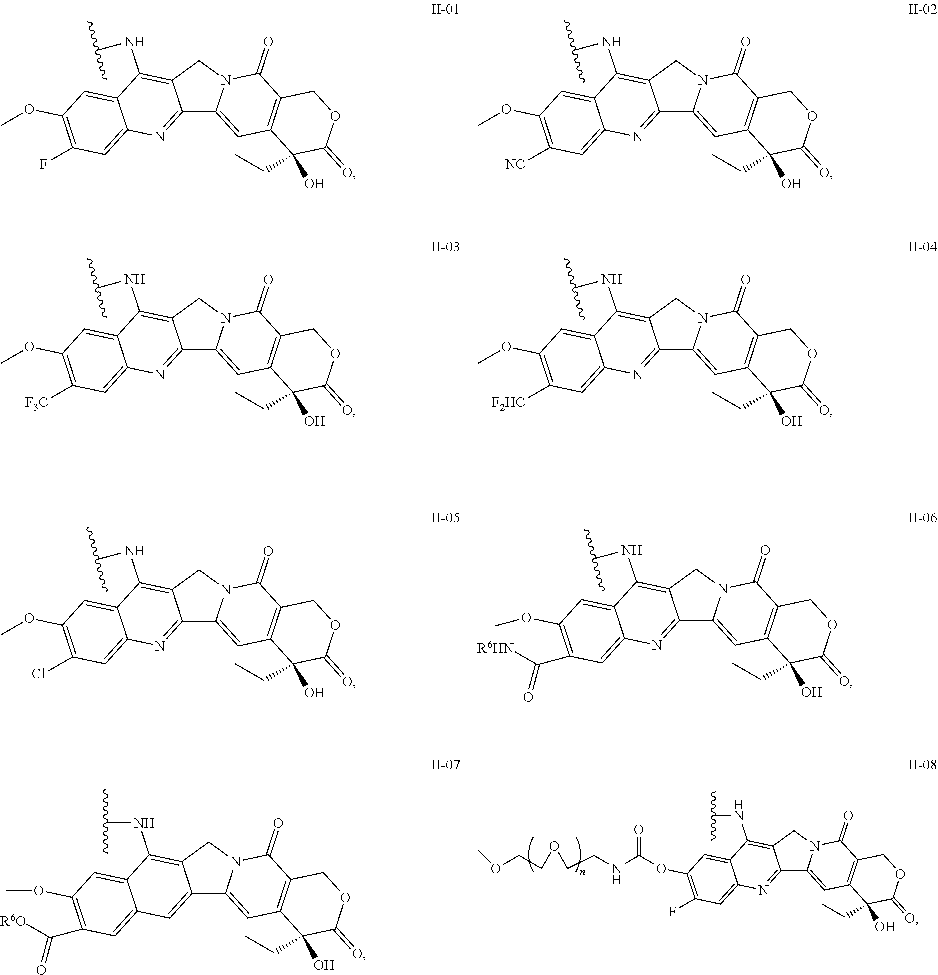 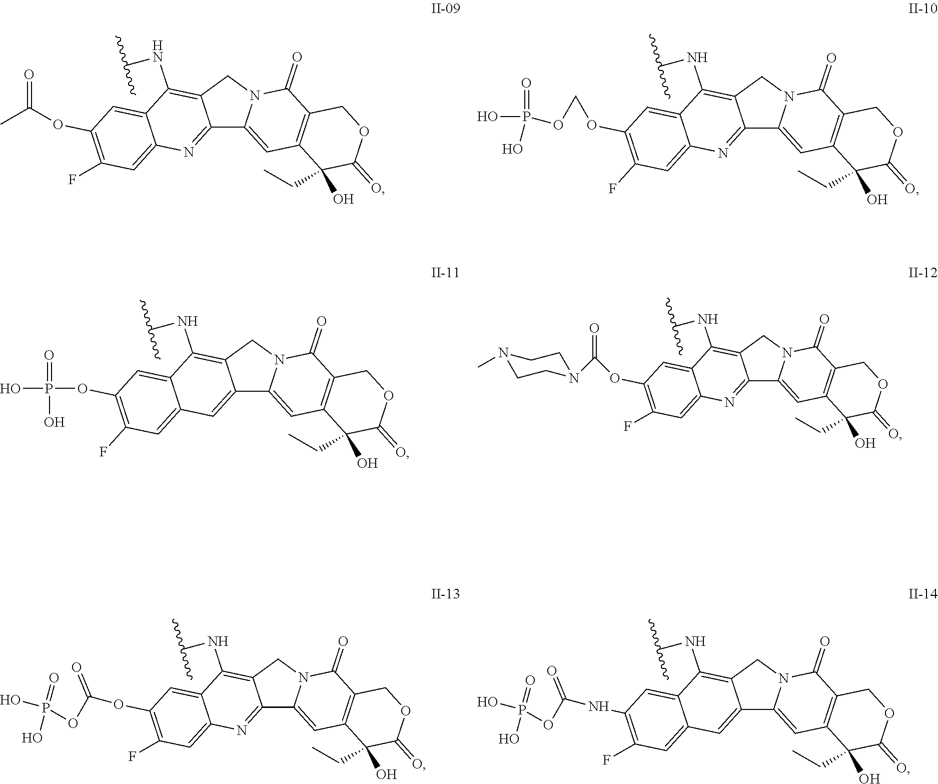 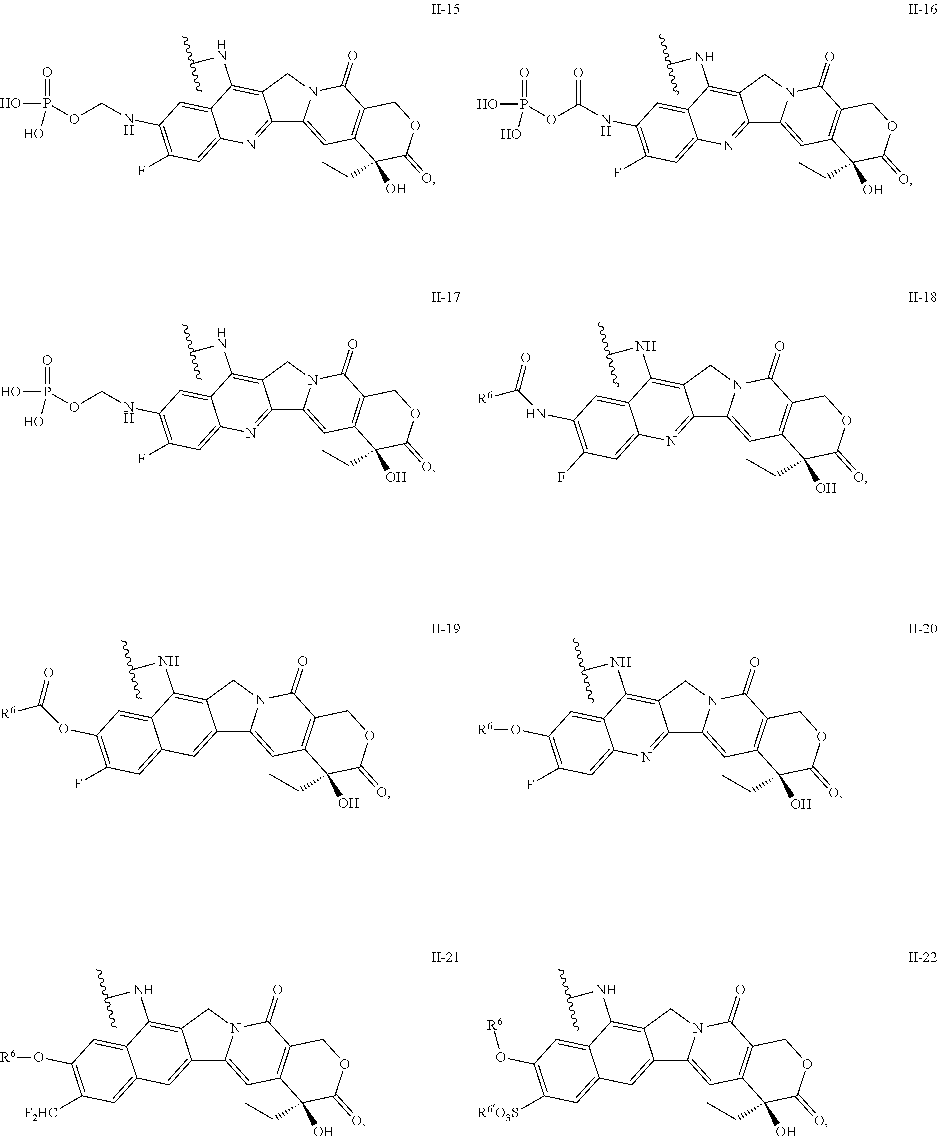 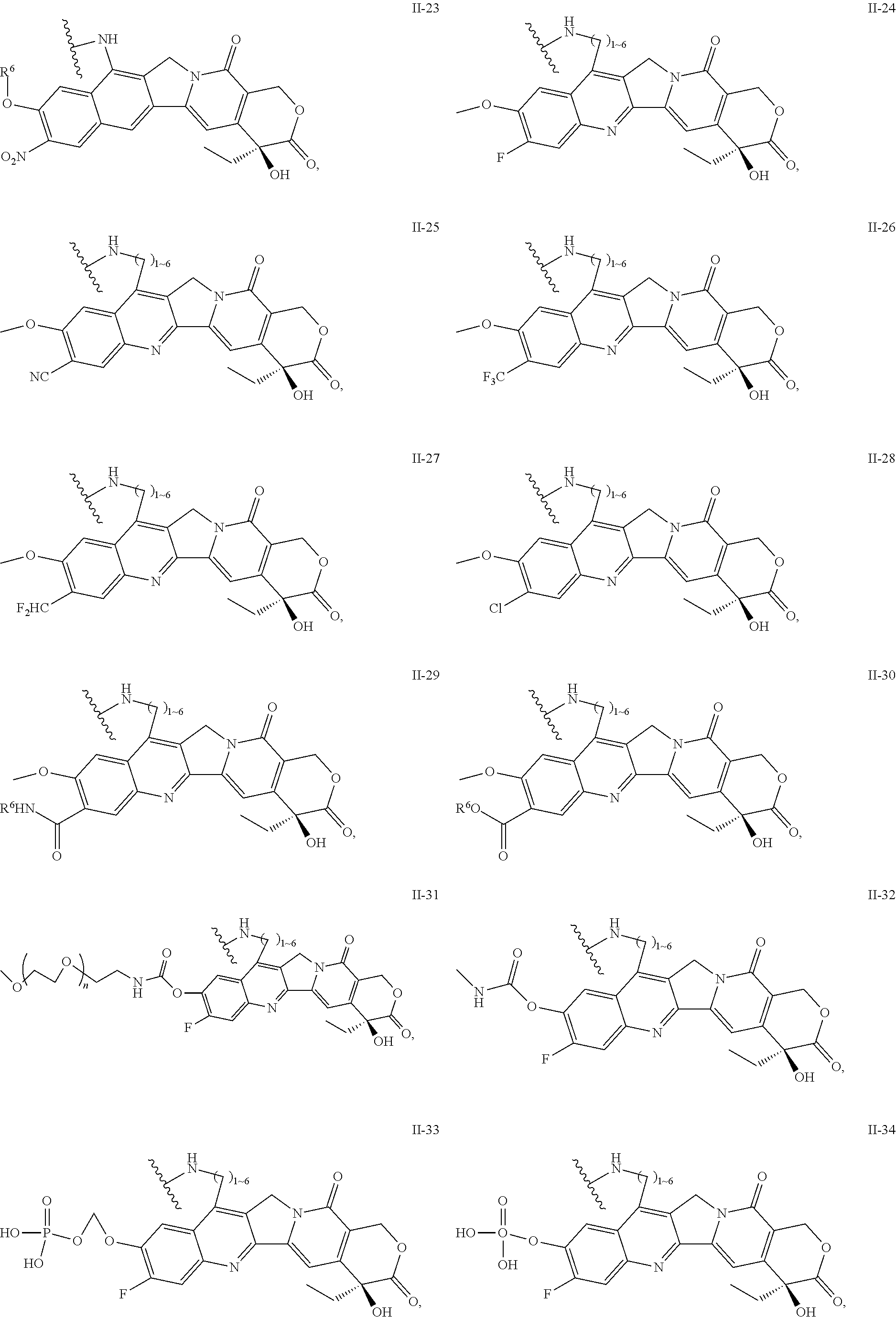 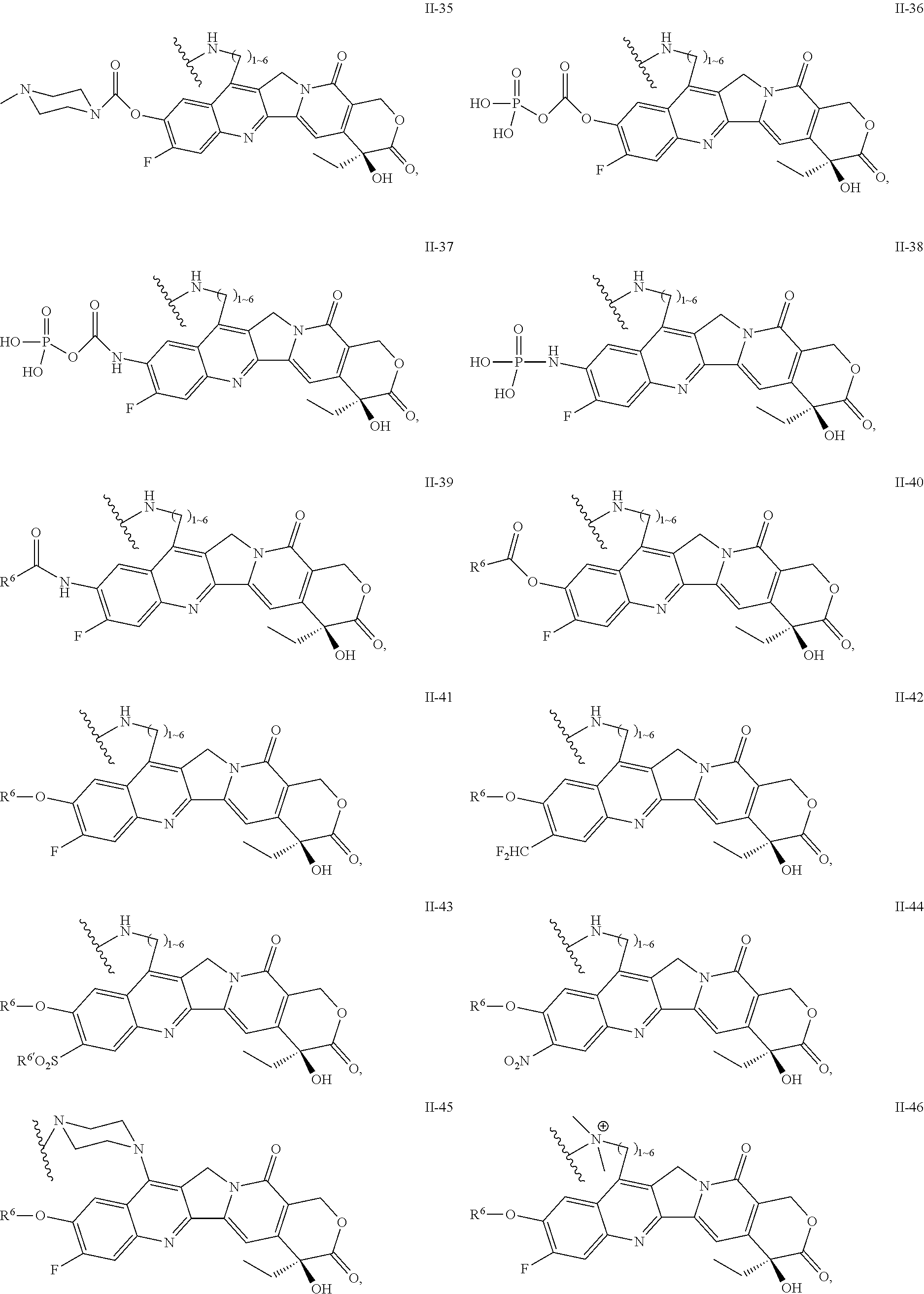 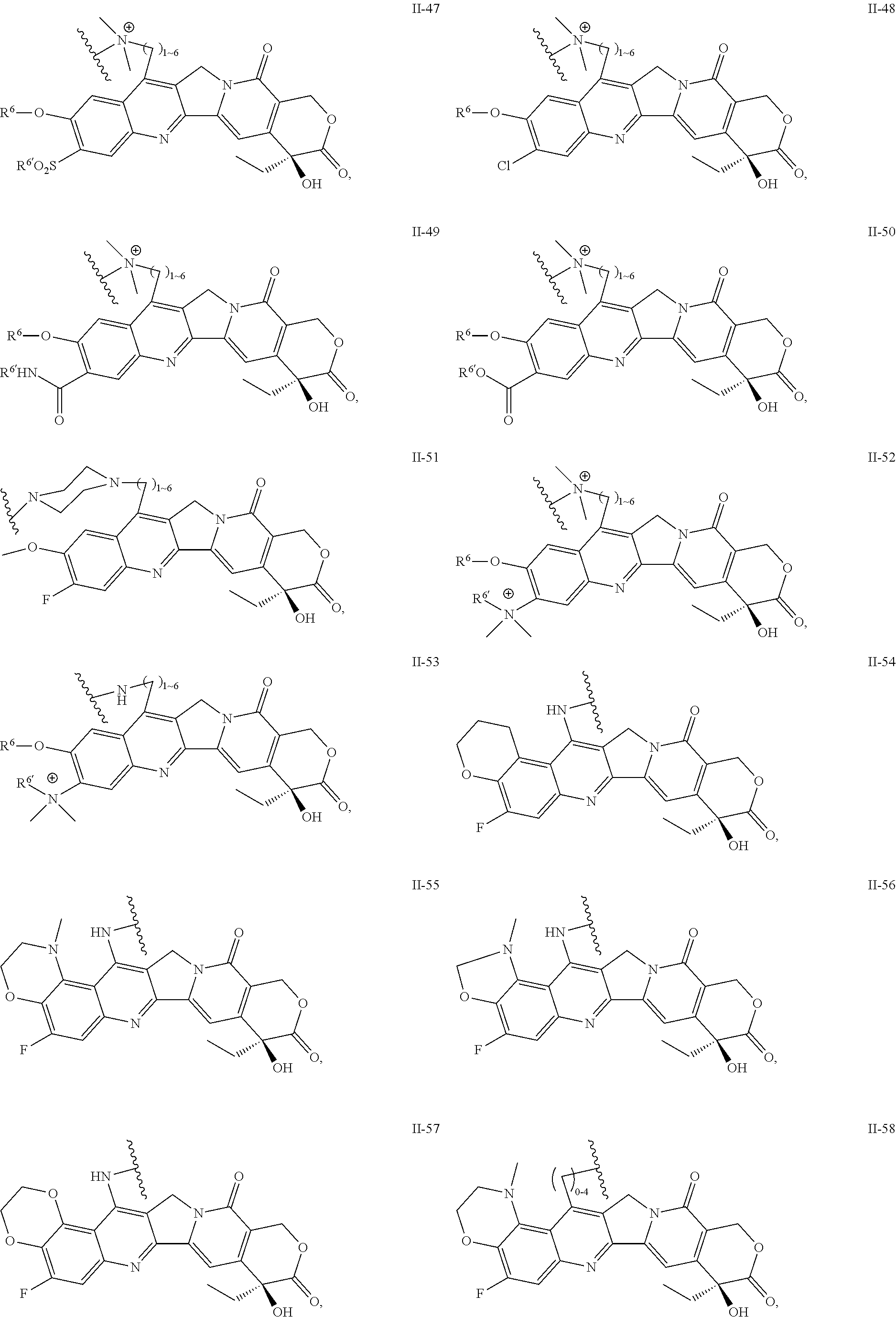 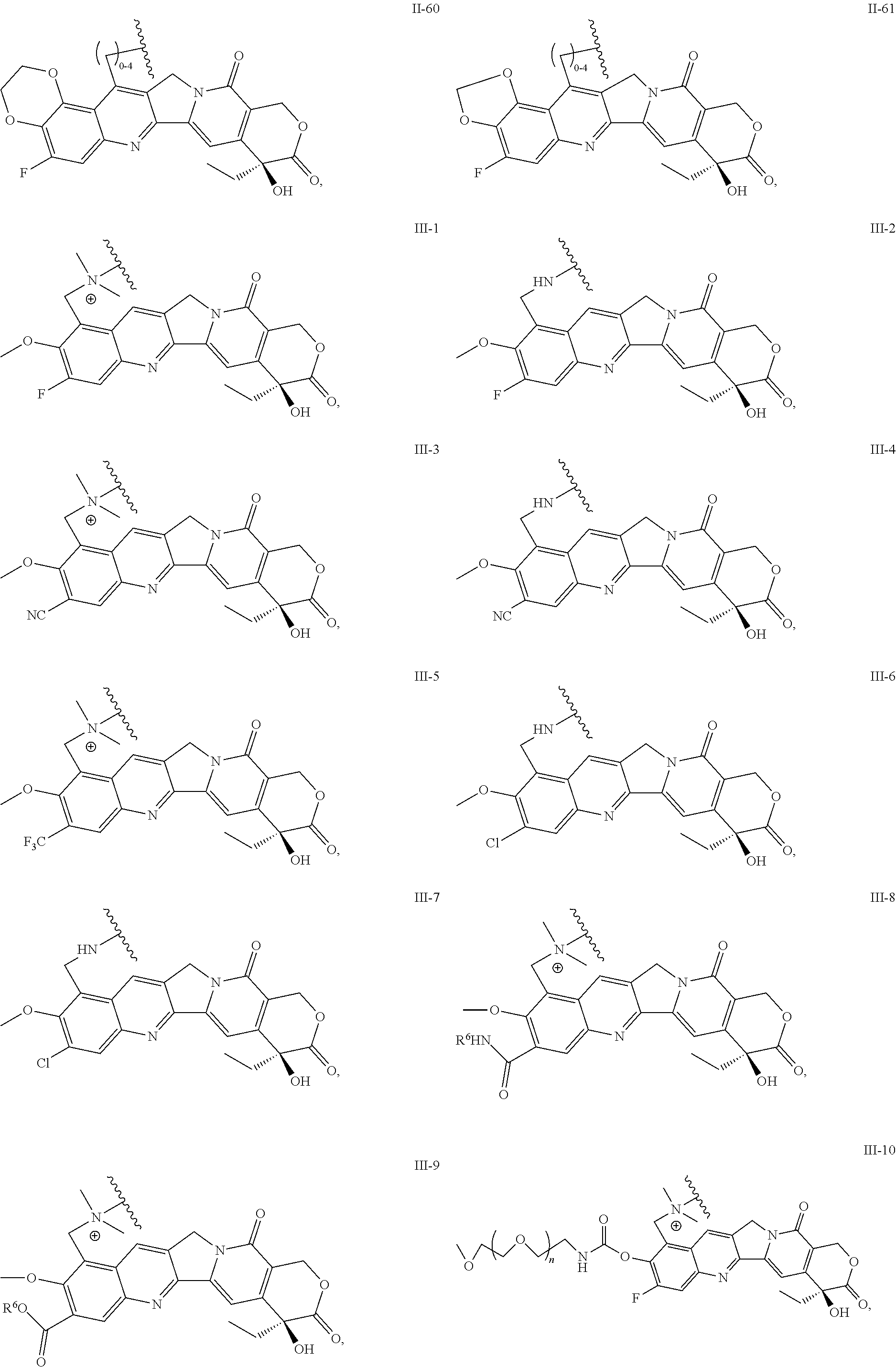 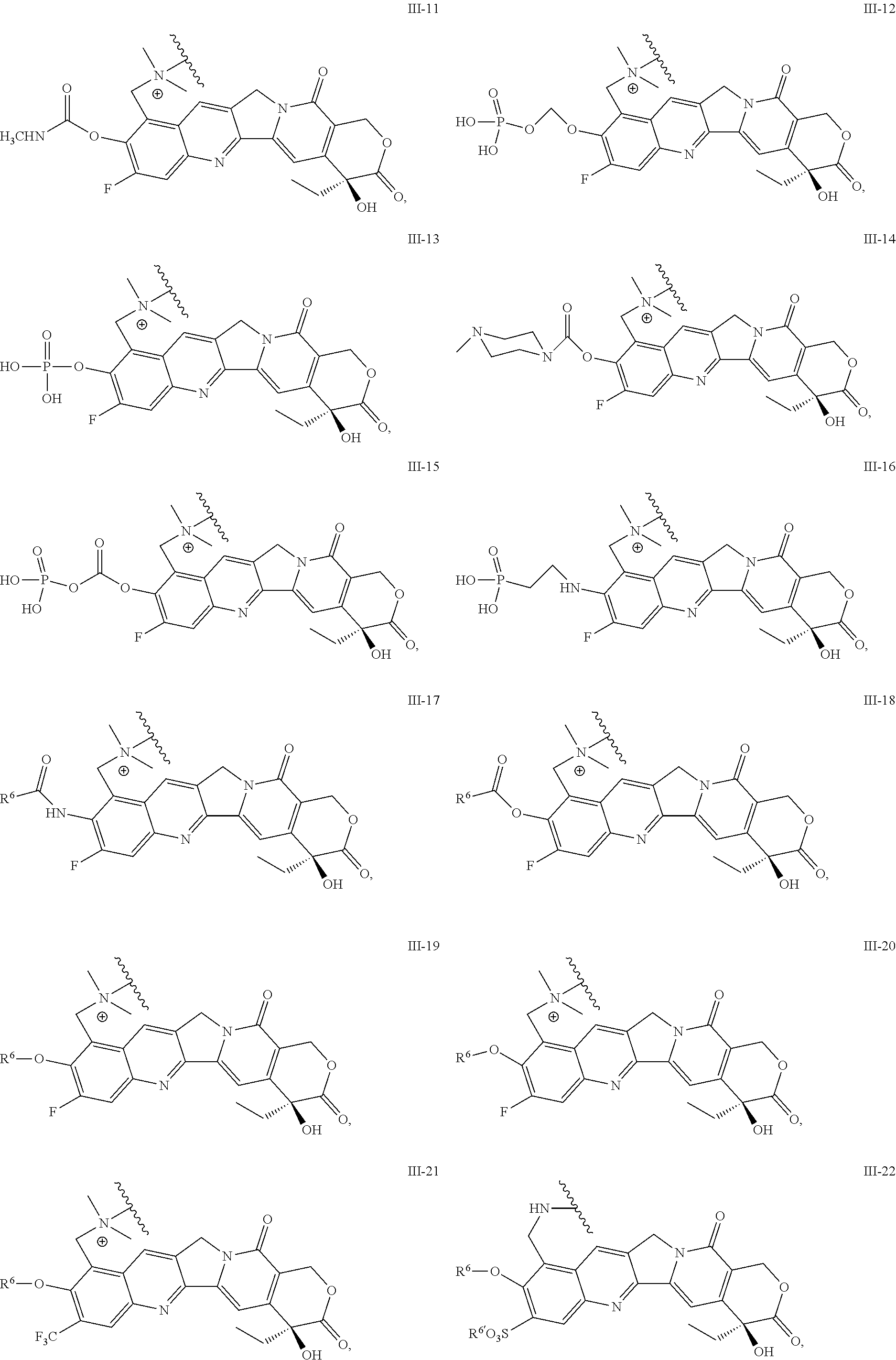 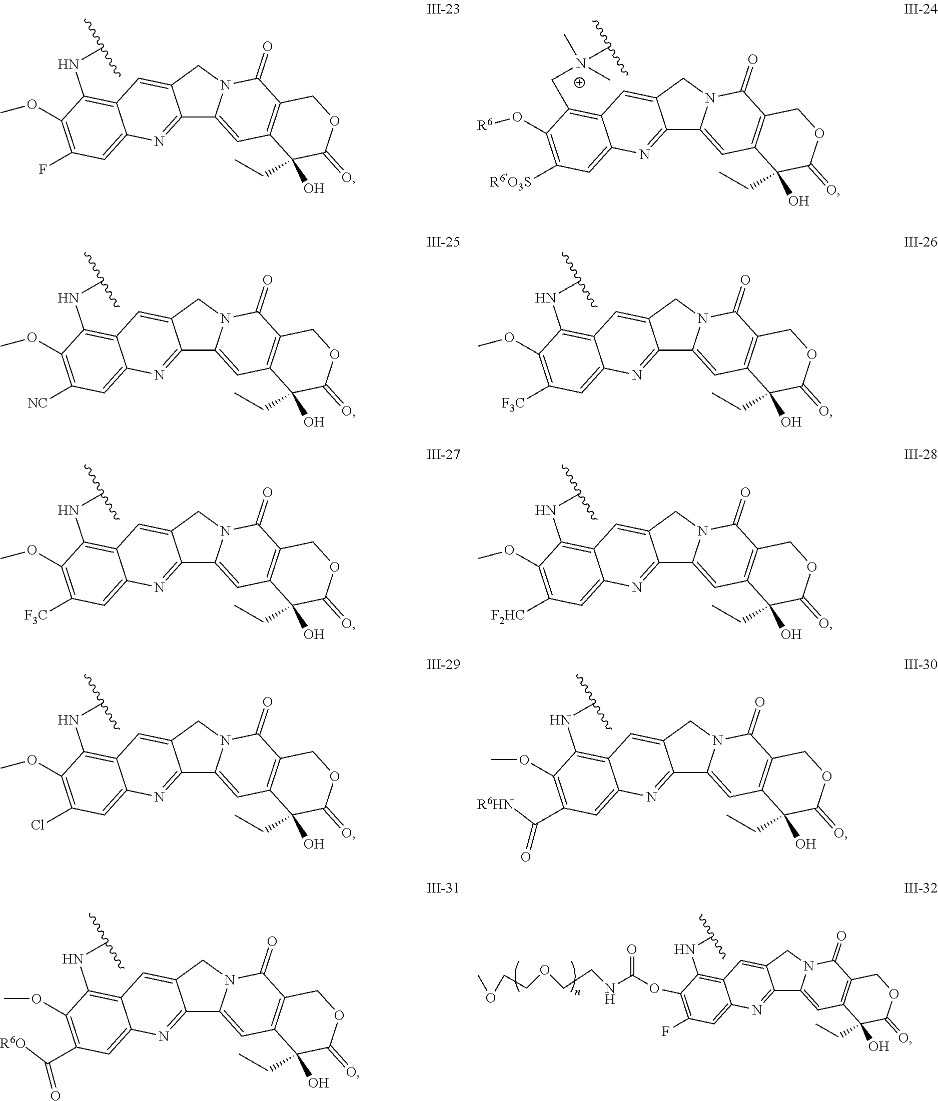 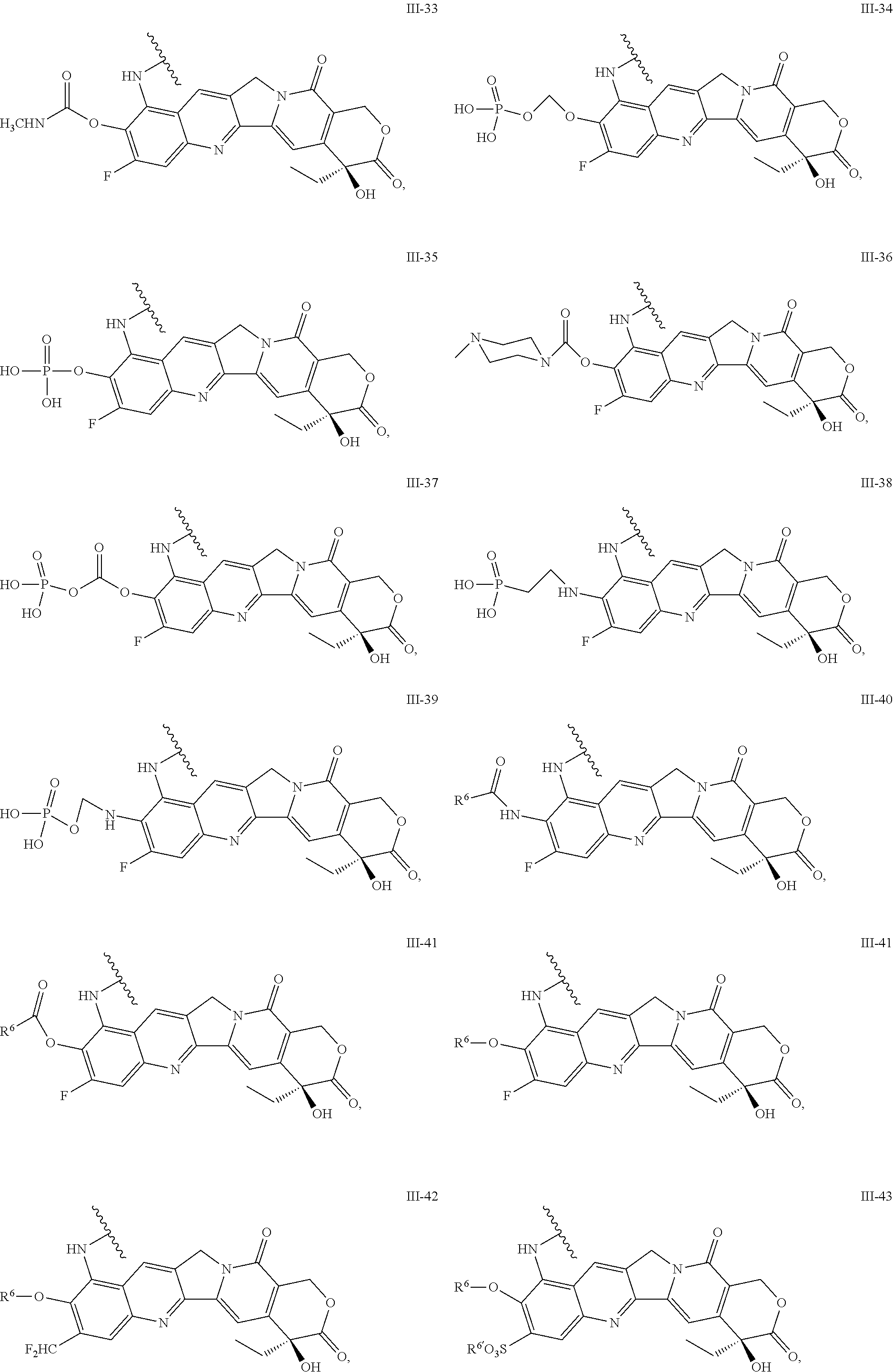 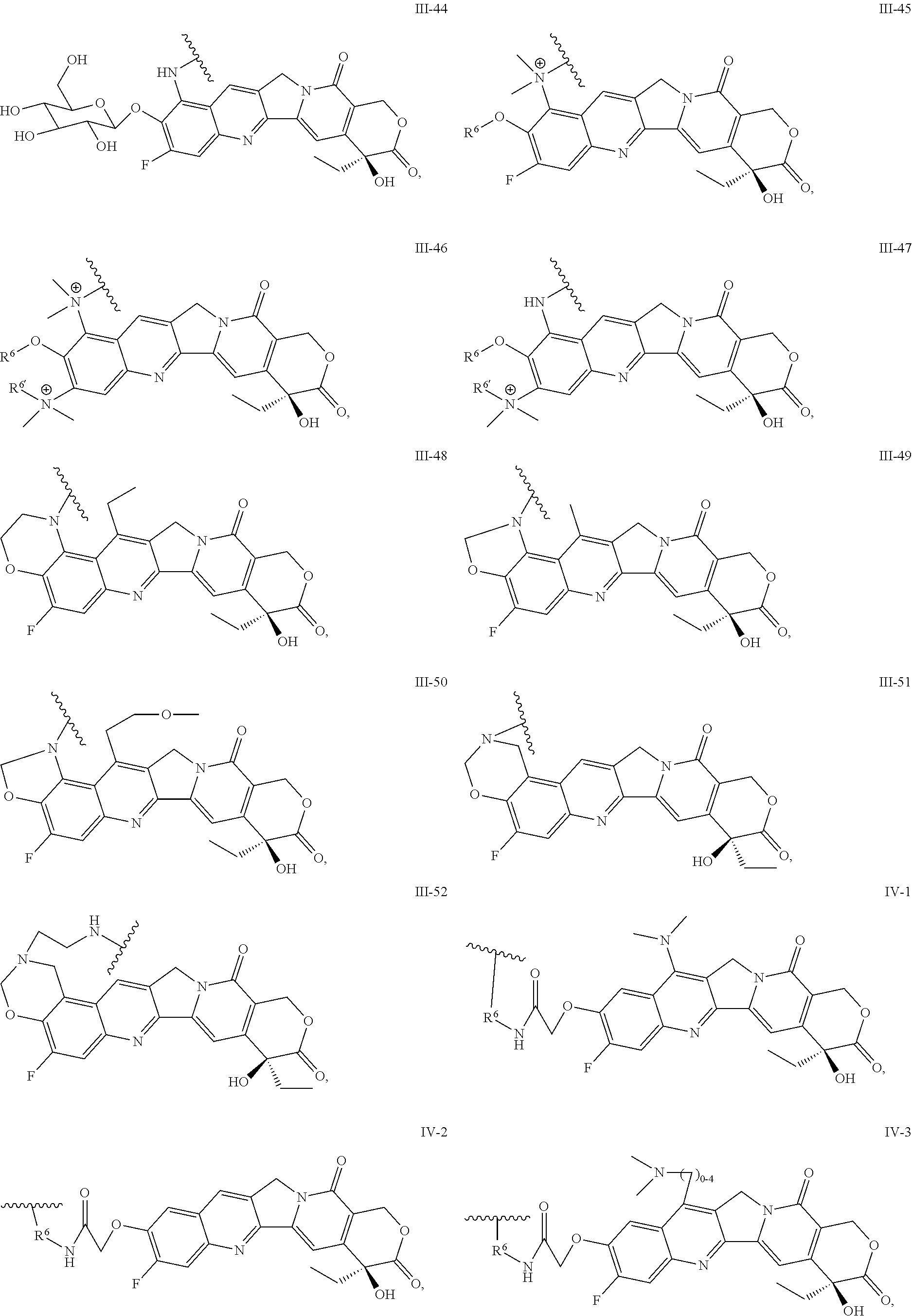 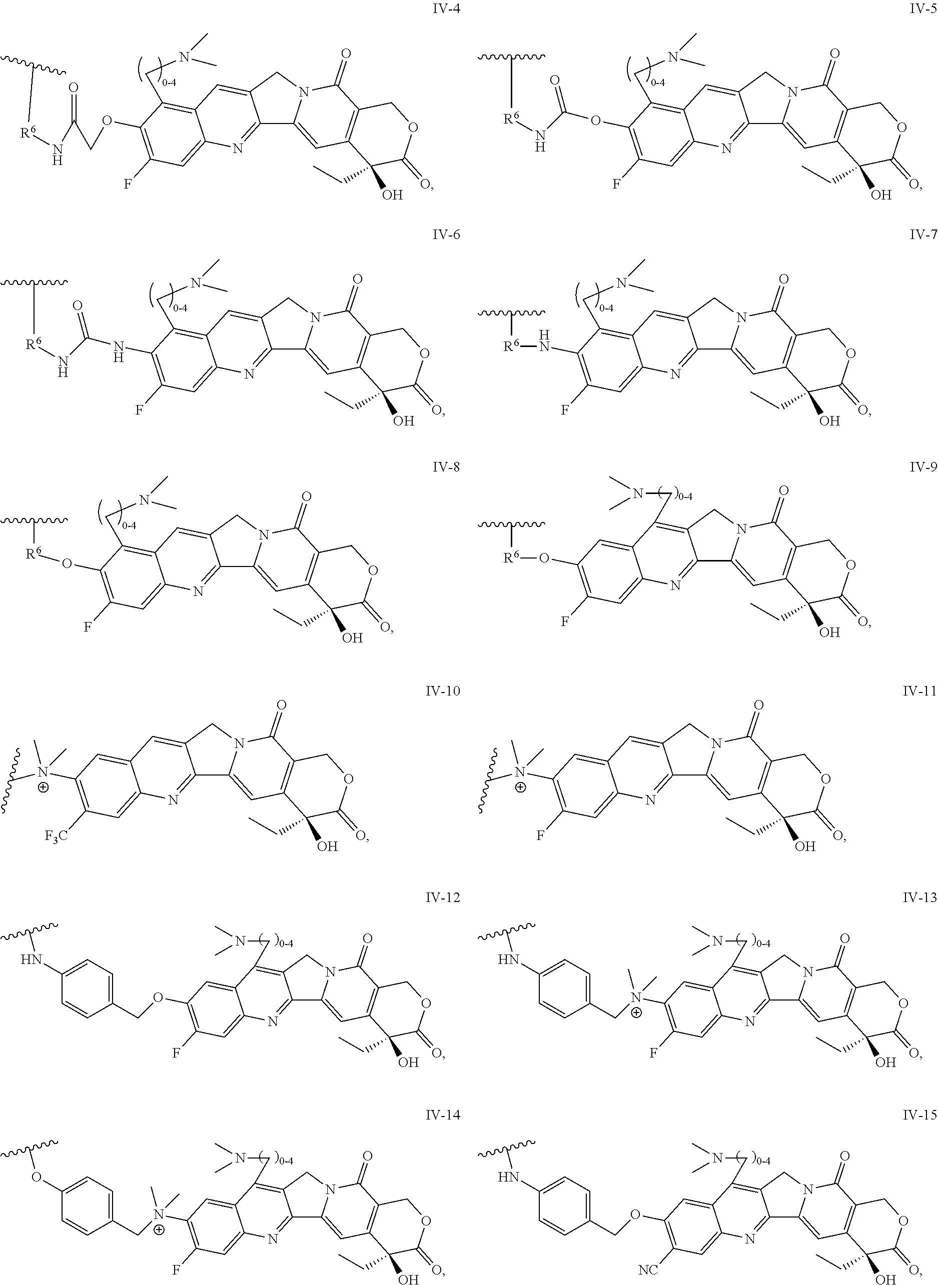 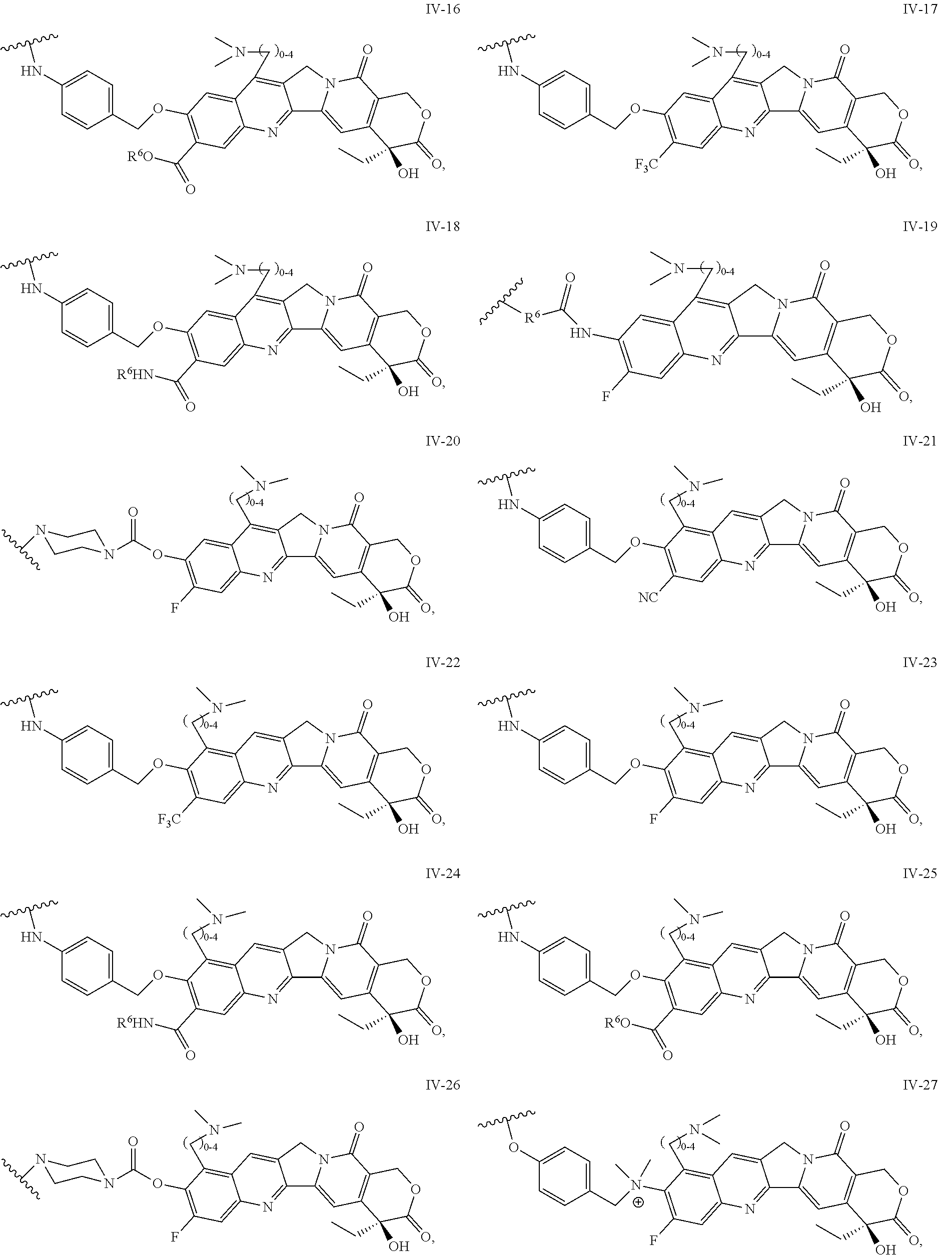 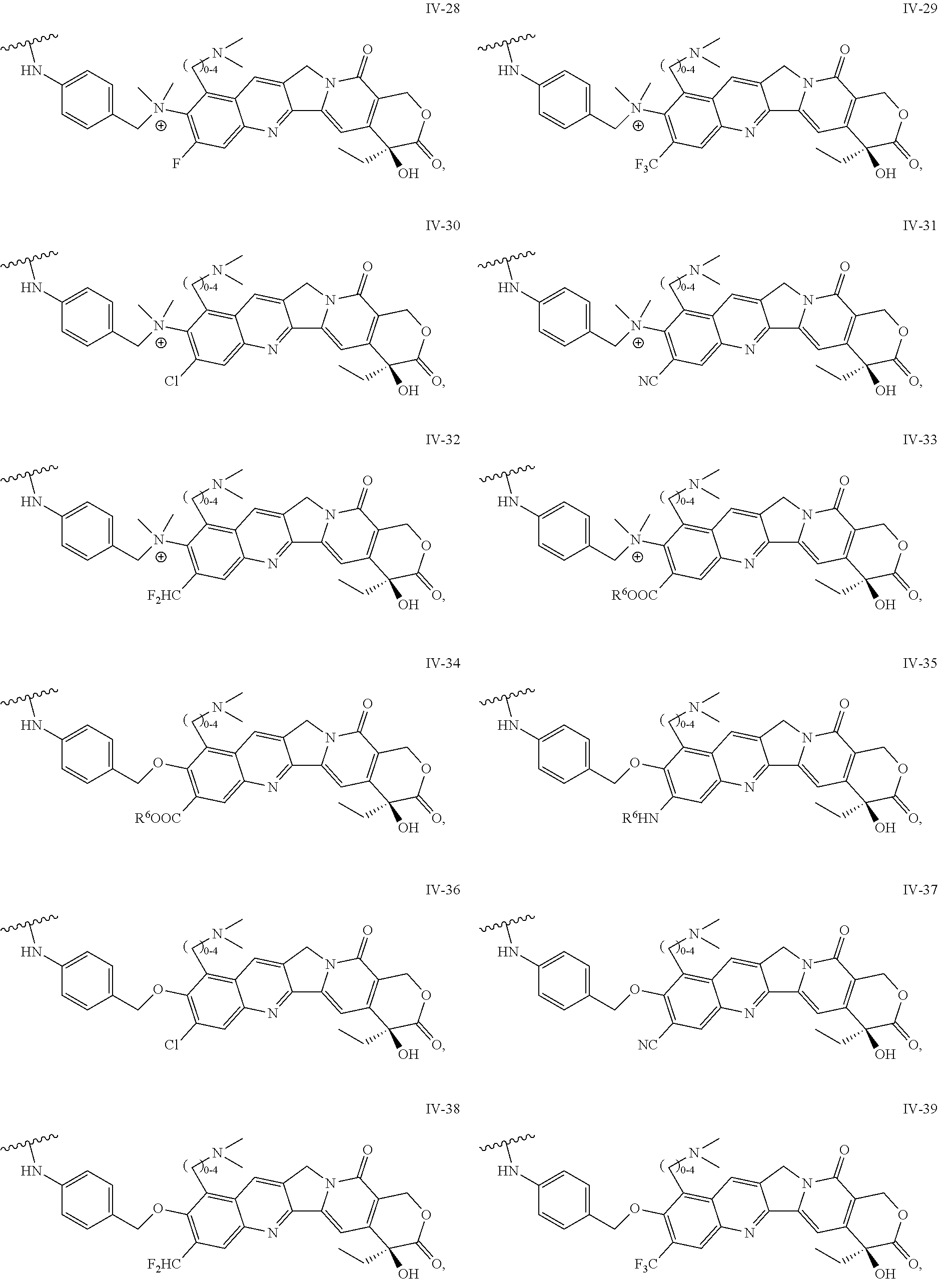 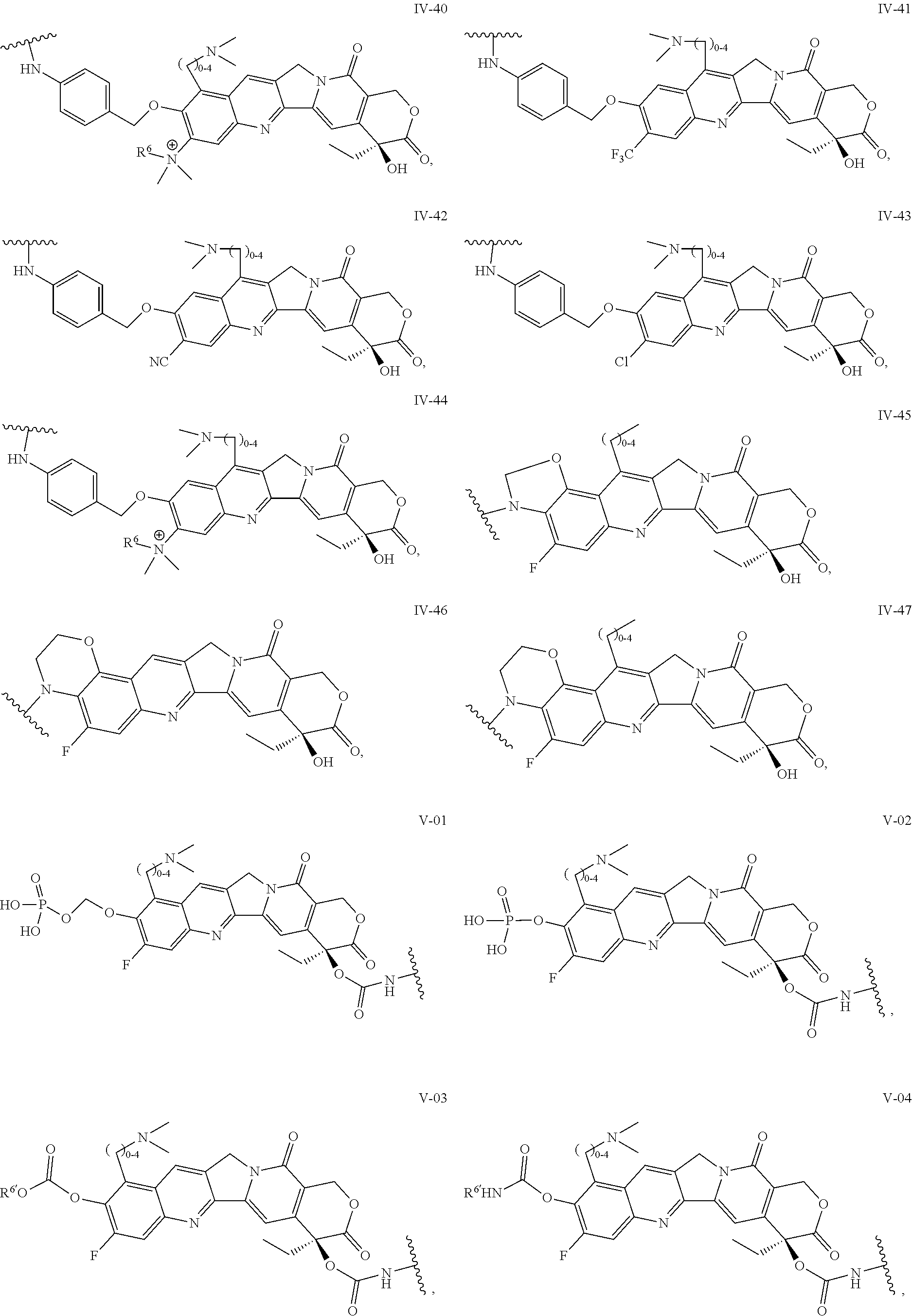 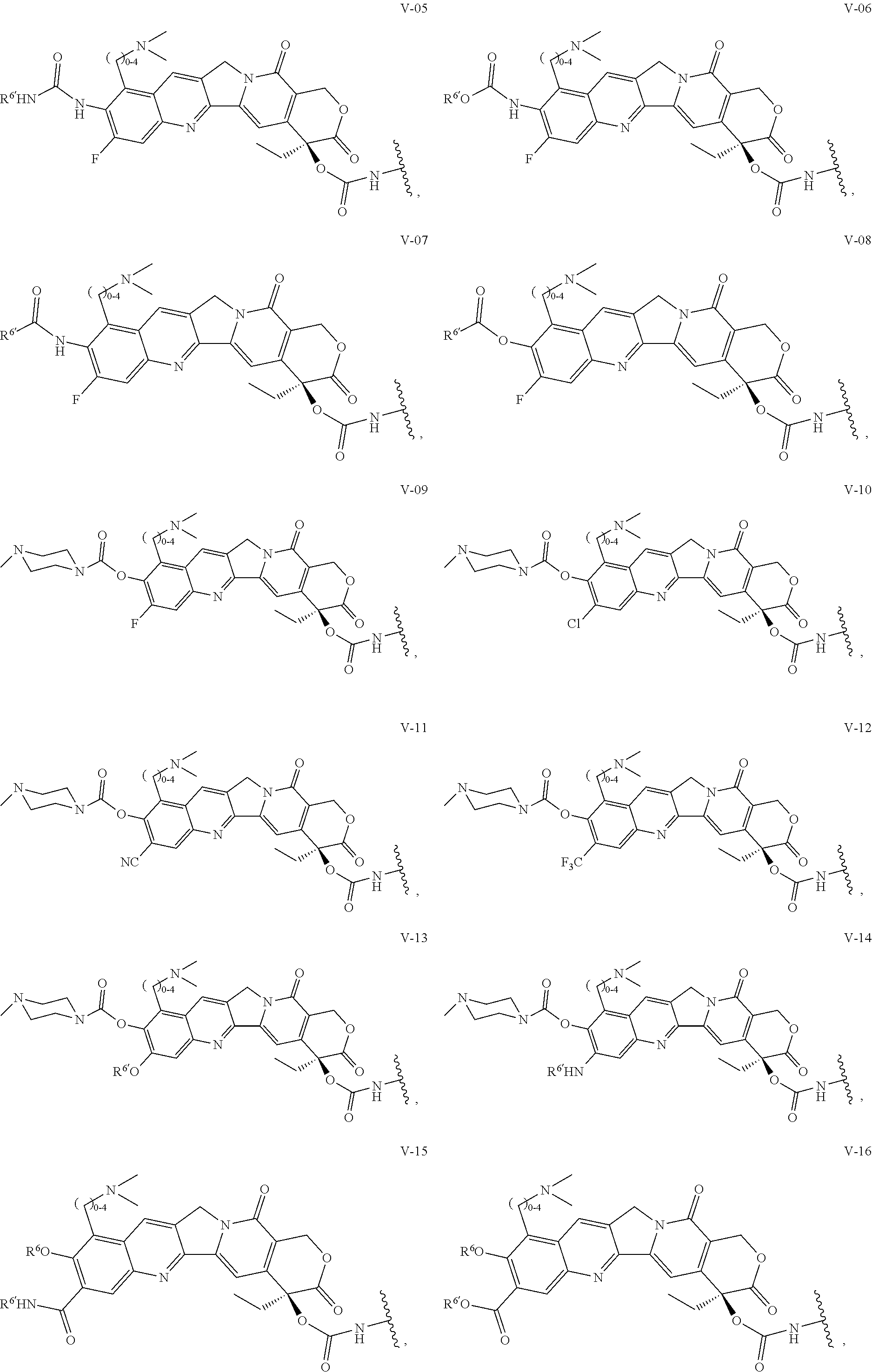 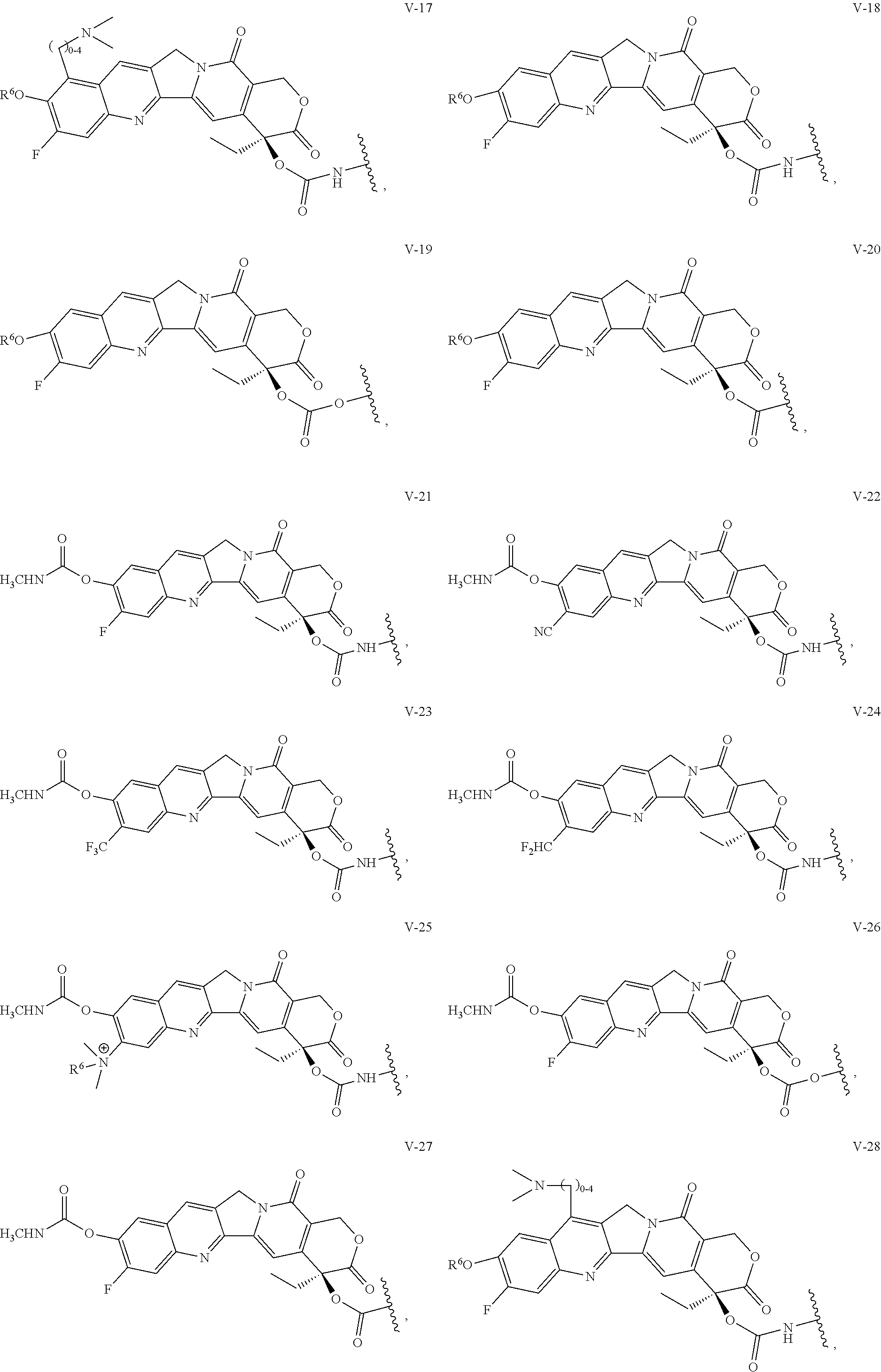 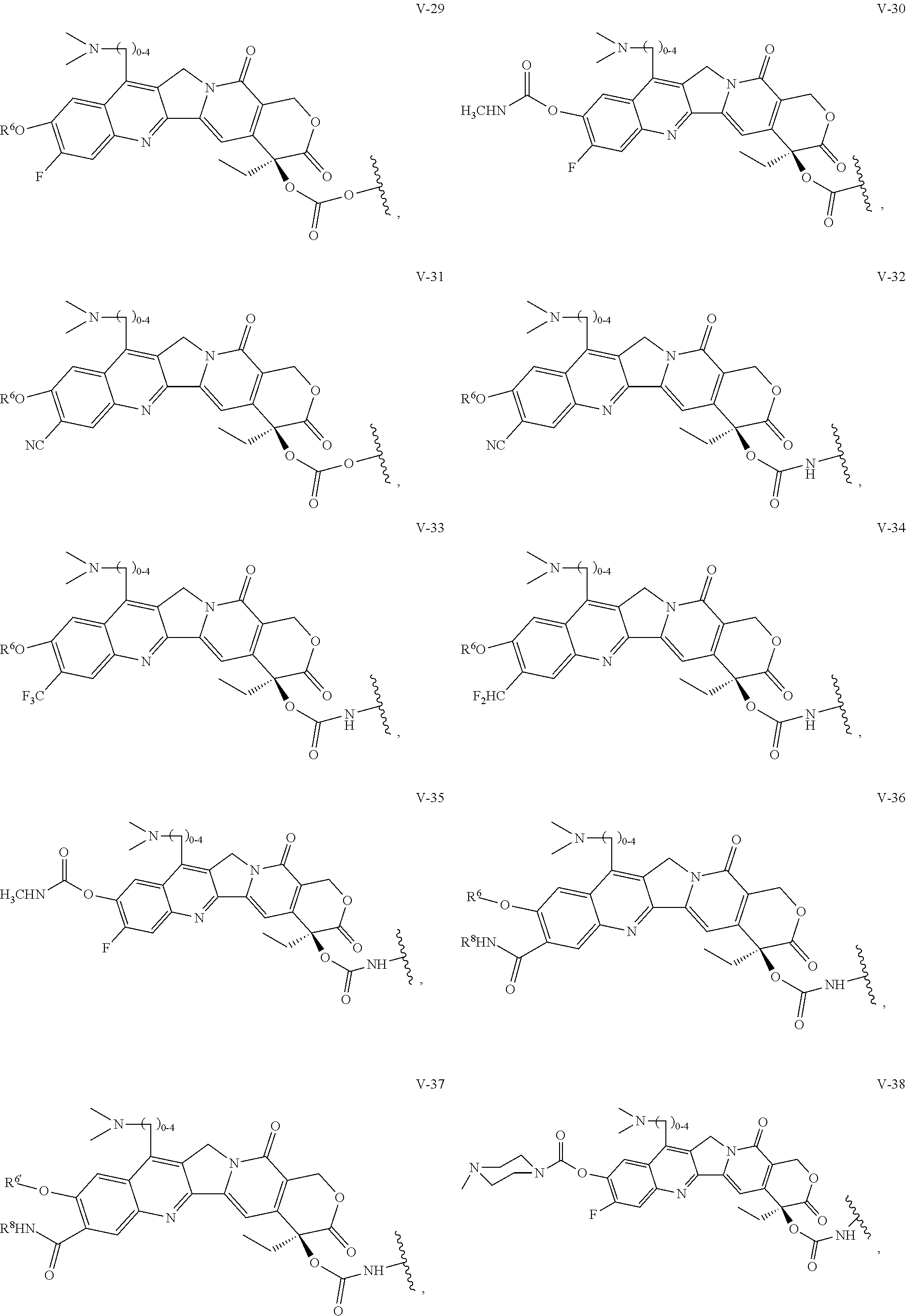 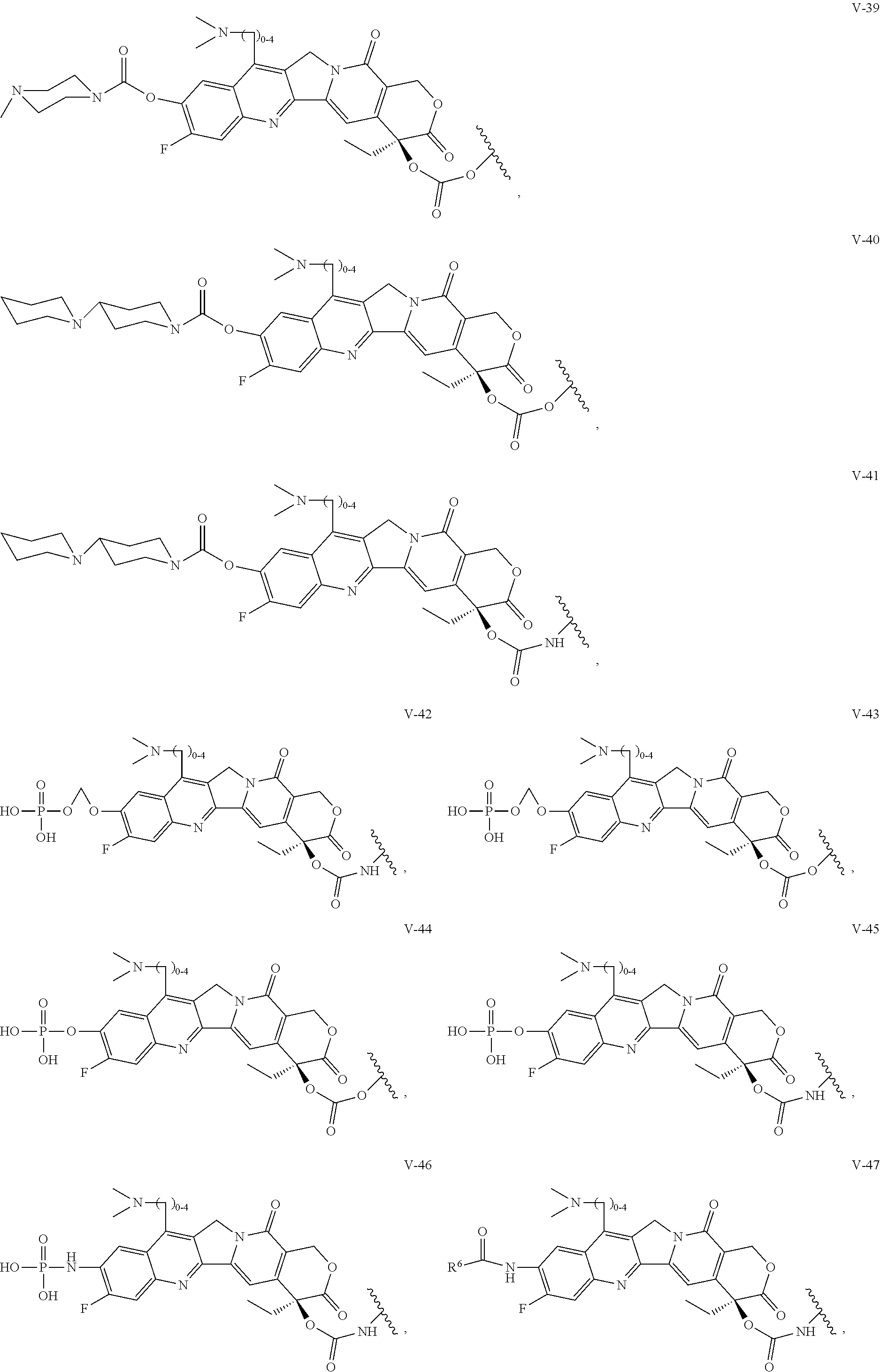 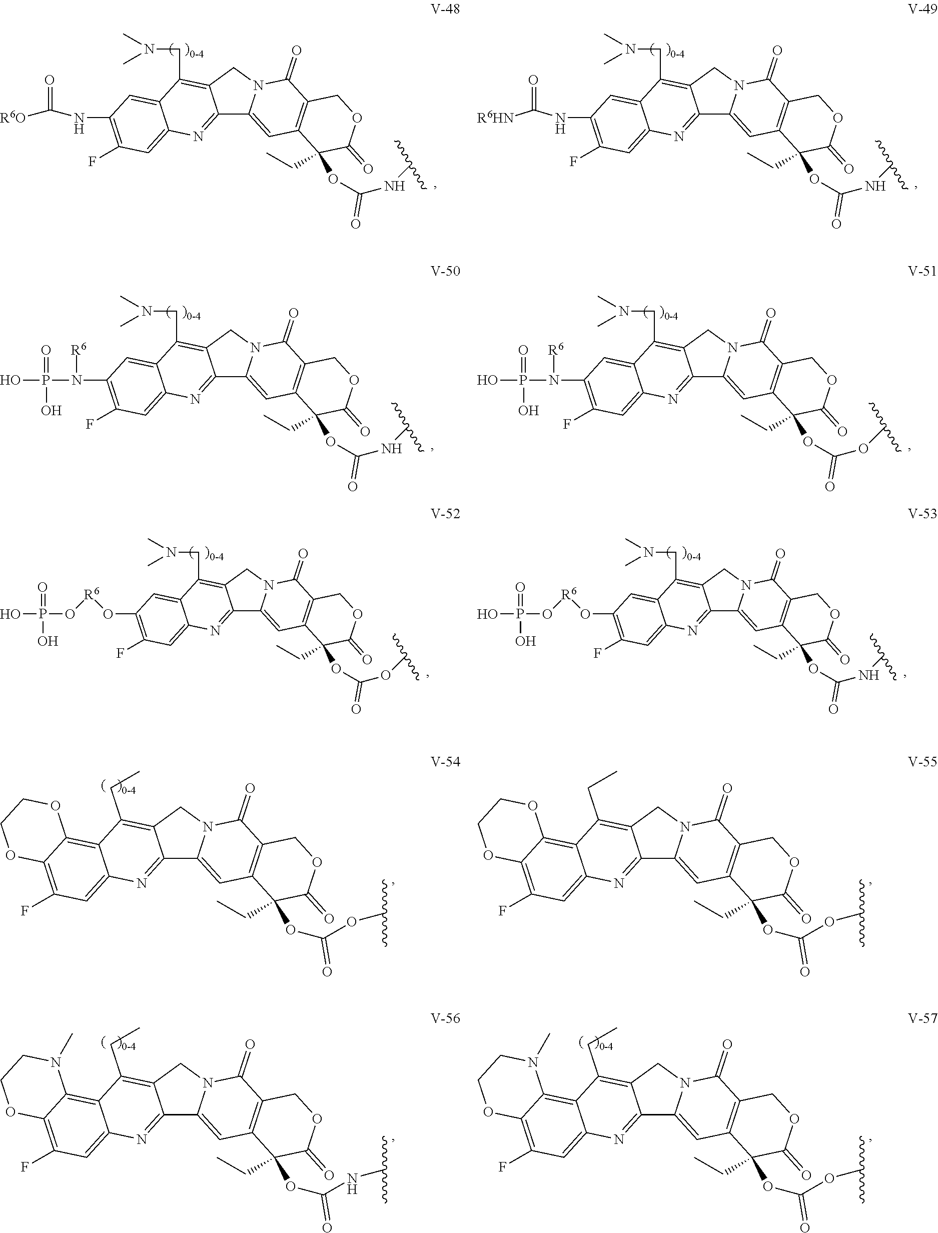 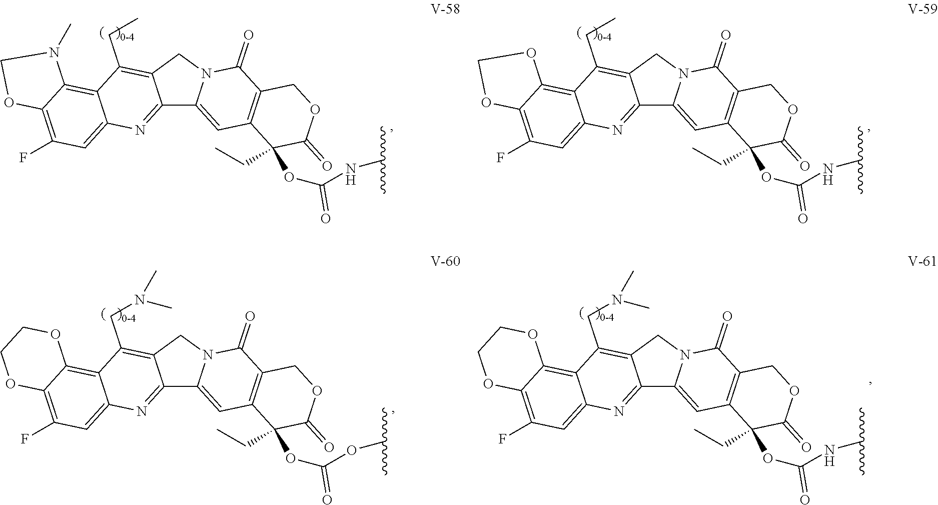
or a pharmaceutically acceptable salt, hydrate, or hydrated salt thereof; or a polymorphic crystalline structure thereof; or an isotope, optical isomer, racemate, diastereomer or enantiomer thereof;
wherein “ [symbol] ” is the site linked to linker L;
wherein R⁶, and R^{6′} are independently H, C₁-C₆ alkyl, alkyl alcohol, alkyl amine (including primary, secondary, tertiary amine, or quaternary ammonium) or alkyl carboxylic acid; C₂-C₆ heteroalkyl, alkylcycloalkyl, heterocycloalkyl, heterocyclic, cycloalkyl, heteroalkylcycloalkyl, alkylcarbonyl, alkyl ether, alkyl ester, alkyl amide or an amino acid; or a pharmaceutical salt.

32. The process according to claim 30, wherein the conjugatable compound has formula (IIq-9), (IIq-10), (IIq-11), (IIq-12), (IIq-13), (IIq-14), (IIq-15), or (IIq-16) below: 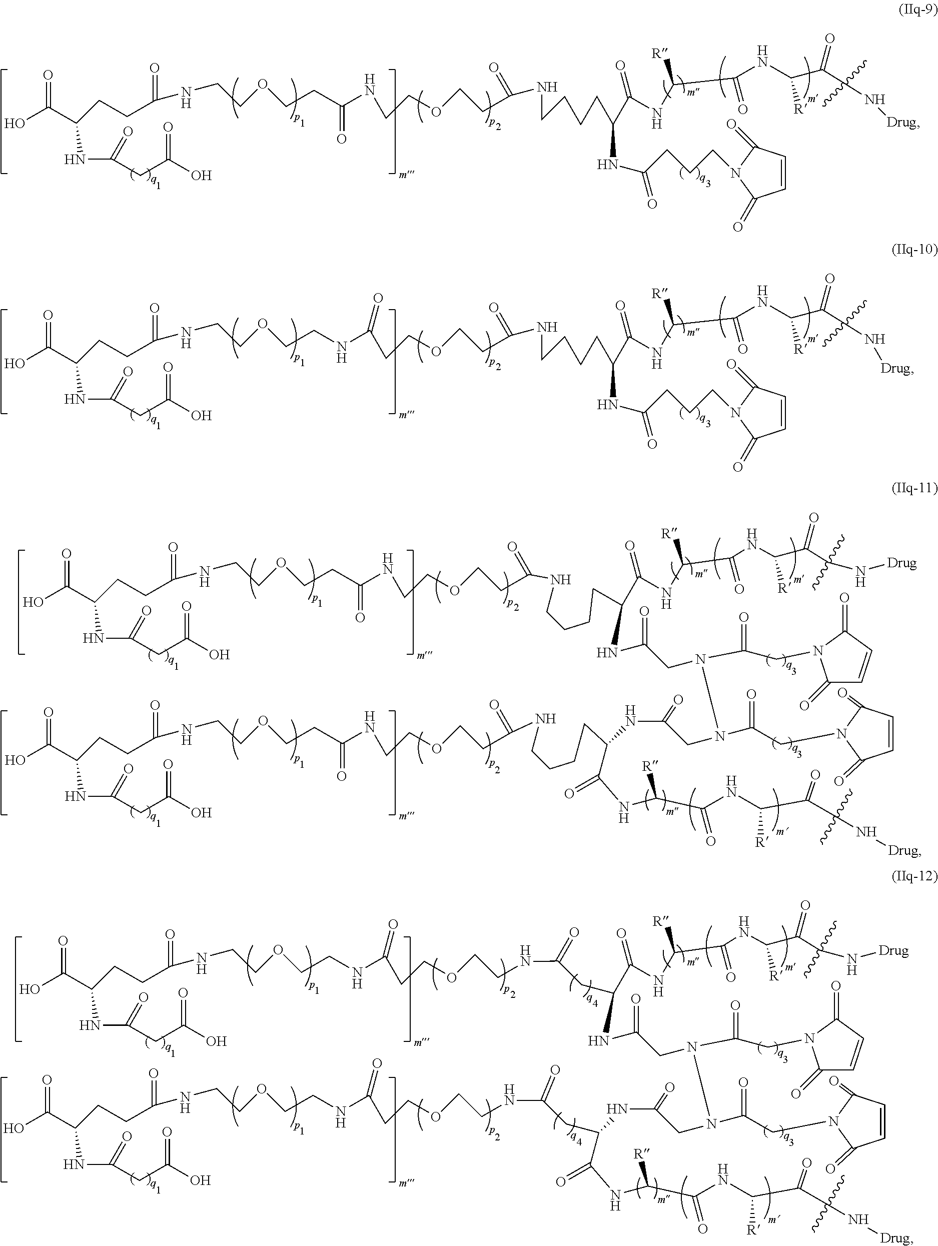 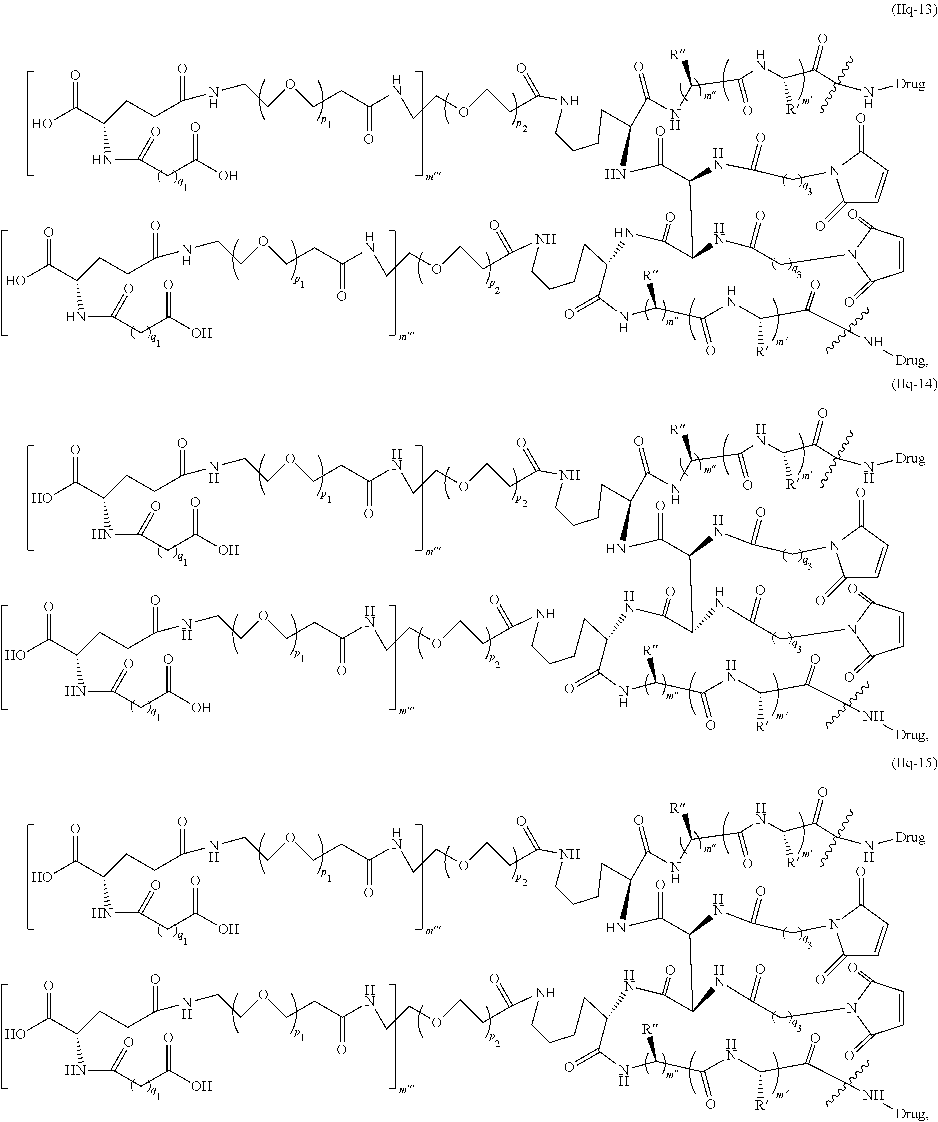 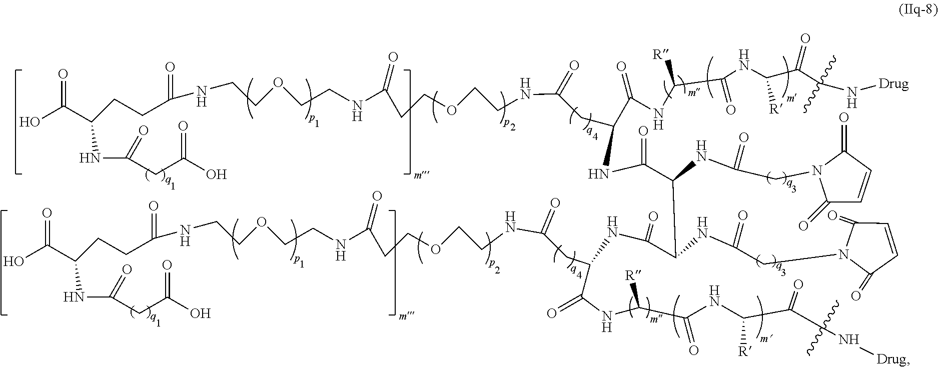
wherein R′ and R″ are independently H, Me, Et, ⁱPr, ⁱBu, Bz (CH₂C₆H₅), CH₂COOH, CH₂CH₂COOH, CH₂CONH₂, CH₂CH₂CONH₂, CH₂CH₂CH₂CH₂NH₂, CH₂CH₂SCH₃, CH₂OH, CH₂CH₂CH₂NHC(═NH)NH₂, CH(OH)CH₃, CH₂C₆H₄OH, or CH₂C₃N₂H₃; p₁ and p₂ are independently 0-24; q₁ is 1-18; q₃ is 0-6; q₄ is 0-4; m′ and m″ are independently 0-6; m″′ is 0 or 1; NH-Drug is a compound of formula II-1 to II-61, III-1 to III-51, IV-1 to IV-47, and V-1 to V-61; and [symbol] is a site linked to NH-Drug.

33. The conjugate according to claim 22, having one of structures of C1-005, C1-008, C1-021, C1-022, C1-029, C1-031, C1-035, C1-041, C1-042, C1-043, C1-047, C1-050, C1-056, C1-061, C1-064, C1-070, C1-075, C1-081, C1-086, C1-088, C1-090, C1-094, C1-099, C1-102, C1-110, C1-102, C1-110, C1-113, C1-114, C1-119a, C1-119b, C1-123, C1-127, C1-131, C1-137a, C1-137b, C1-140, C1-147a, C1-147b, C1-151, C1-152, C1-156, C1-157, C1-158, C1-159a, C1-159b, C1-165, C1-166, C1-168, C1-170a, C1-170b, C1-177, C1-188, C1-200, C1-208, C1-213, C1-226, C1-238, C1-243, C1-247, C1-262a, C1-262b, C1-262c, C1-262d, C1-266, C1-285a to C1-285z, C1-285a₁ to C1-285i₁, C1-291a to C1-291z, C1-291a₁ to C1-291i₁, C1-297a to C1-297z, C1-297a₁ to C1-297i₁, C1-305, C1-306, C1-311, C1-362, C1-397, C1-402, C1-407, C1-411, C1- 414, C1-419, C1-424, C1-428, C1-436, C2-005, C3-005, C2-008, C3-008, C2-021, C3-021, C2-022, C3-022, C2-029, C3-029, C2-031, C3-031, C2-035, C3-035, C2-041, C3-041, C2-042, C3-042, C2-043, C3-043, C2-047, C3-047, C2-050, C3-050, C2-056, C3-056, C2-061, C3-061, C2-064, C3-064, C2-070, C3-070, C2-075, C3-075, C2-081, C3-081, C2-086, C3-086, C2-088, C3-088, C2-090, C3-090, C2-094, C3-094, C2-099, C3-099, C2-102, C3-102, C2-110, C3-110, C2-113, C3-113, C2-114, C3-114, C2-119a, C3-119a, C2-119b, C3-119b, C2-123, C3-123, C2-127, C3-127, C2-131, C3-131, C2-137a, C3-137a, C2-137b, C3-137b, C2-140, C3-140, C2-147a, C3-147a, C2-147b, C3-147b, C2-151, C3-151, C2-152, C3-152, C2-156, C3-156, C2-157, C3-157, C2-158, C3-158, C2-159a, C3-159a, C2-159b, C3-159b, C2-165, C3-165, C2-166, C3-166, C2-168, C3-168, C2-170a, C3-170a, C2-170b, C3-170b, C2-177, C3-177, C2-188, C3-188, C2-200, C3-200, C2-208, C3-208, C2-213, C3-213, C2-226, C3-226, C2-238, C3-238, C2-243, C3-243, C2-247, C3-247, C2-262a, C3-262a, C2-262b, C3-262b, C2-262c, C3-262c, C2-262d, C3-262d, C2-266, C3-266, C2-285a to C2-285z, C3-285a to C3-285z, C2-285a₁ to C2-285i₁, C3-285a₁ to C3-285i₁, C2-291a to C2-291z, C2-291a₁ to C2-291i₁, C3-291a to C3-291z, C3-291a₁ to C3-291i₁, C2-297a˜C2-297z, C2-297a₁ to C2-297i₁, C3-297a to C3-297z, C3-297a₁ to C3-297i₁, C2-305, C3-305, C2-306, C3-306, C2-311, C3-311, C2-362, C3-362, C2-397, C3-397, C2-402, C3-402, C2-407, C3-407, C2-411, C3-411, C2-414, C3-414, C2-419, C3-419, C2-424, C3-424, C2-428, C3-428, below: 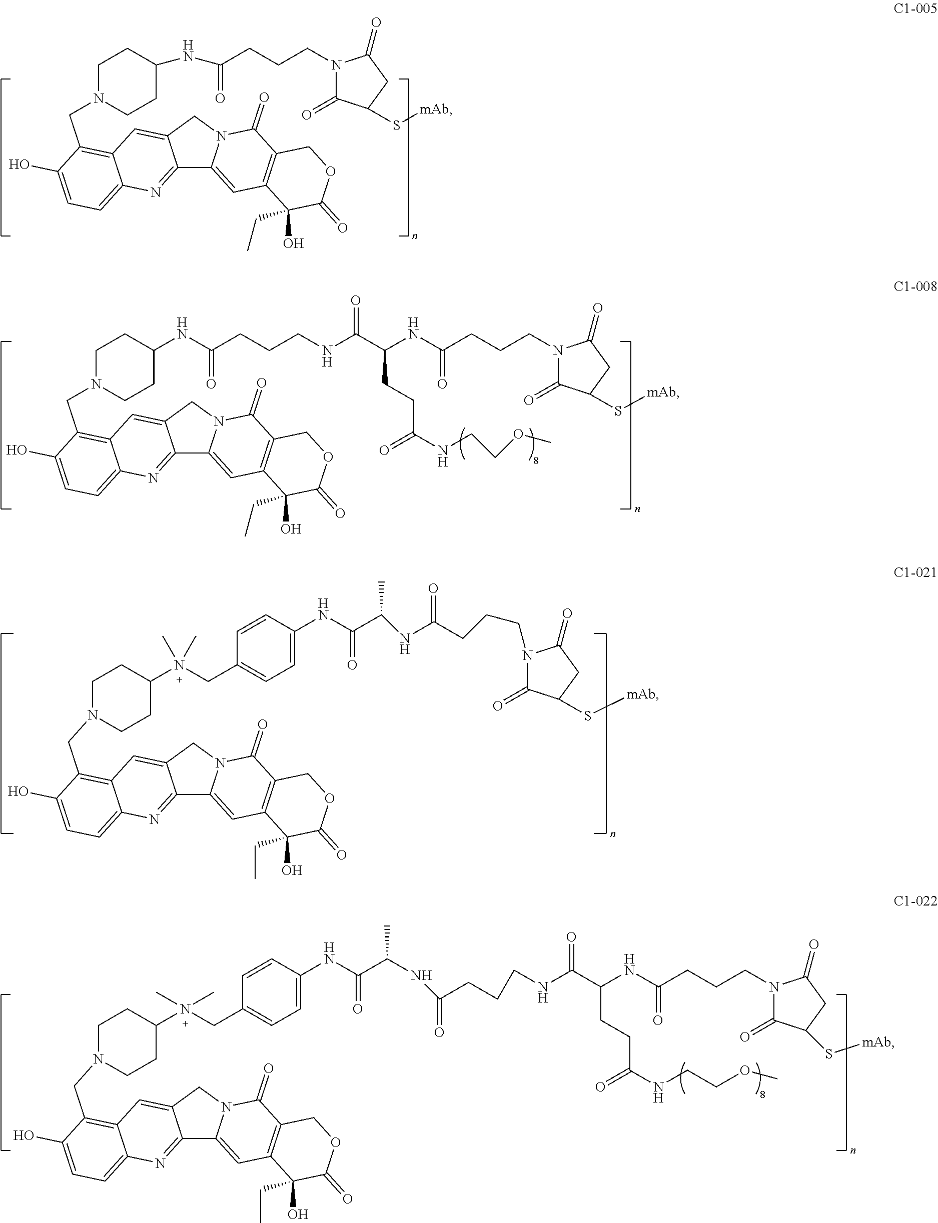 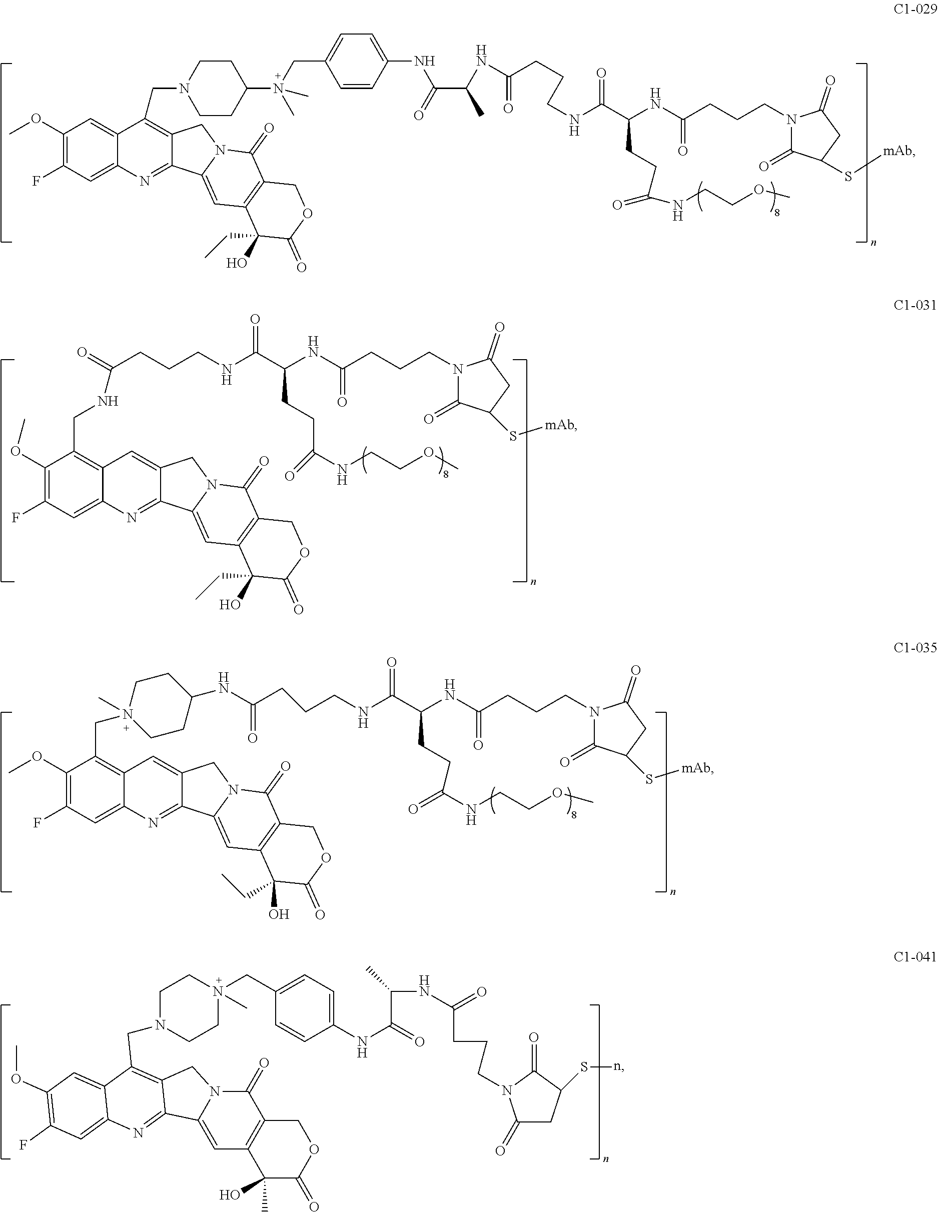 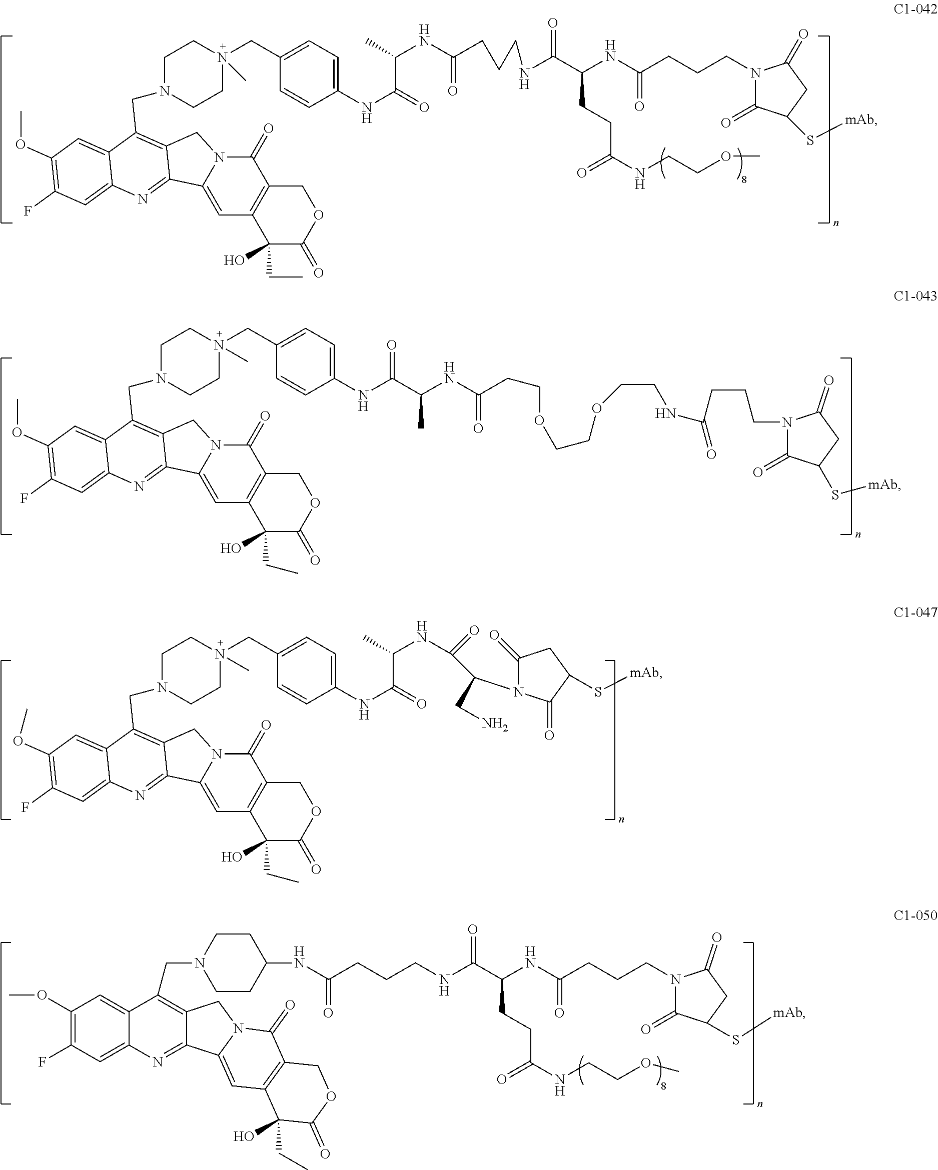 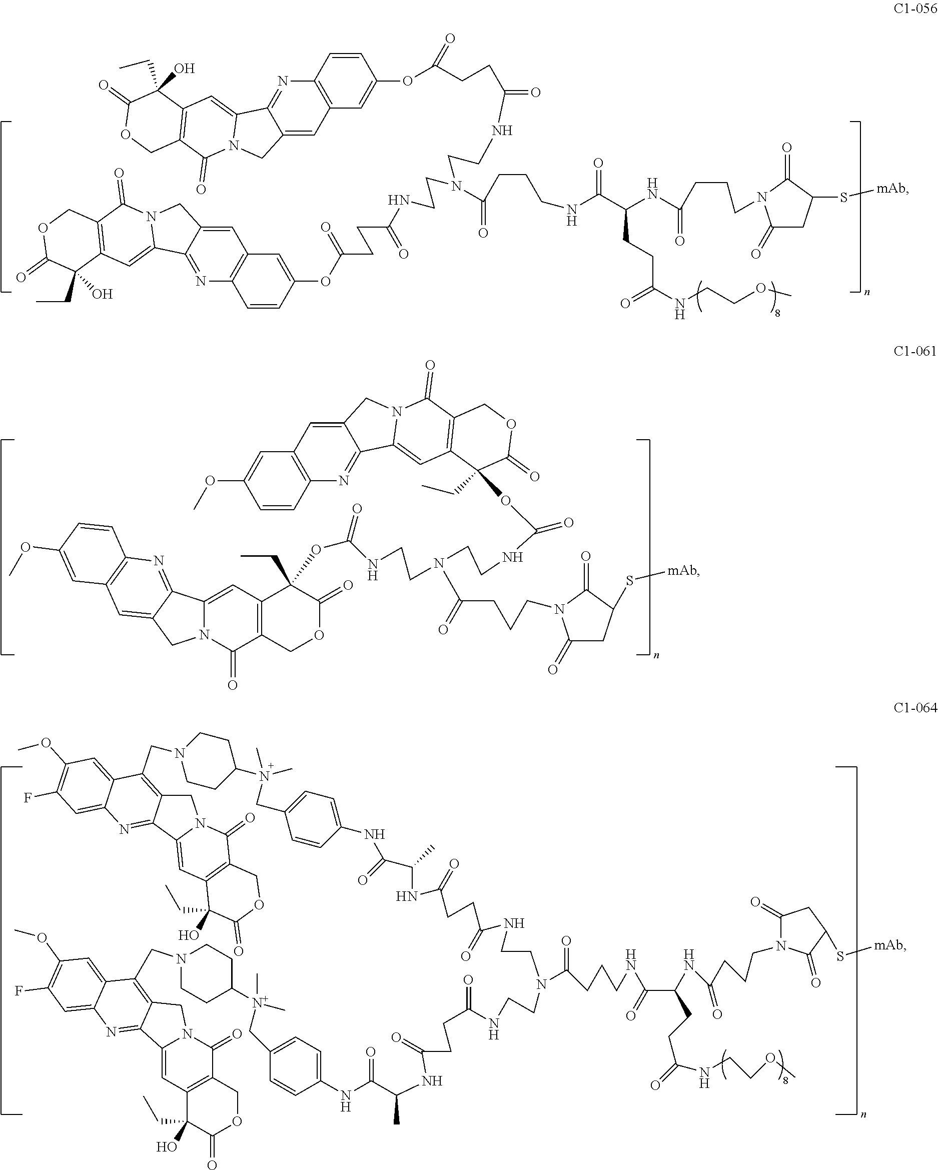 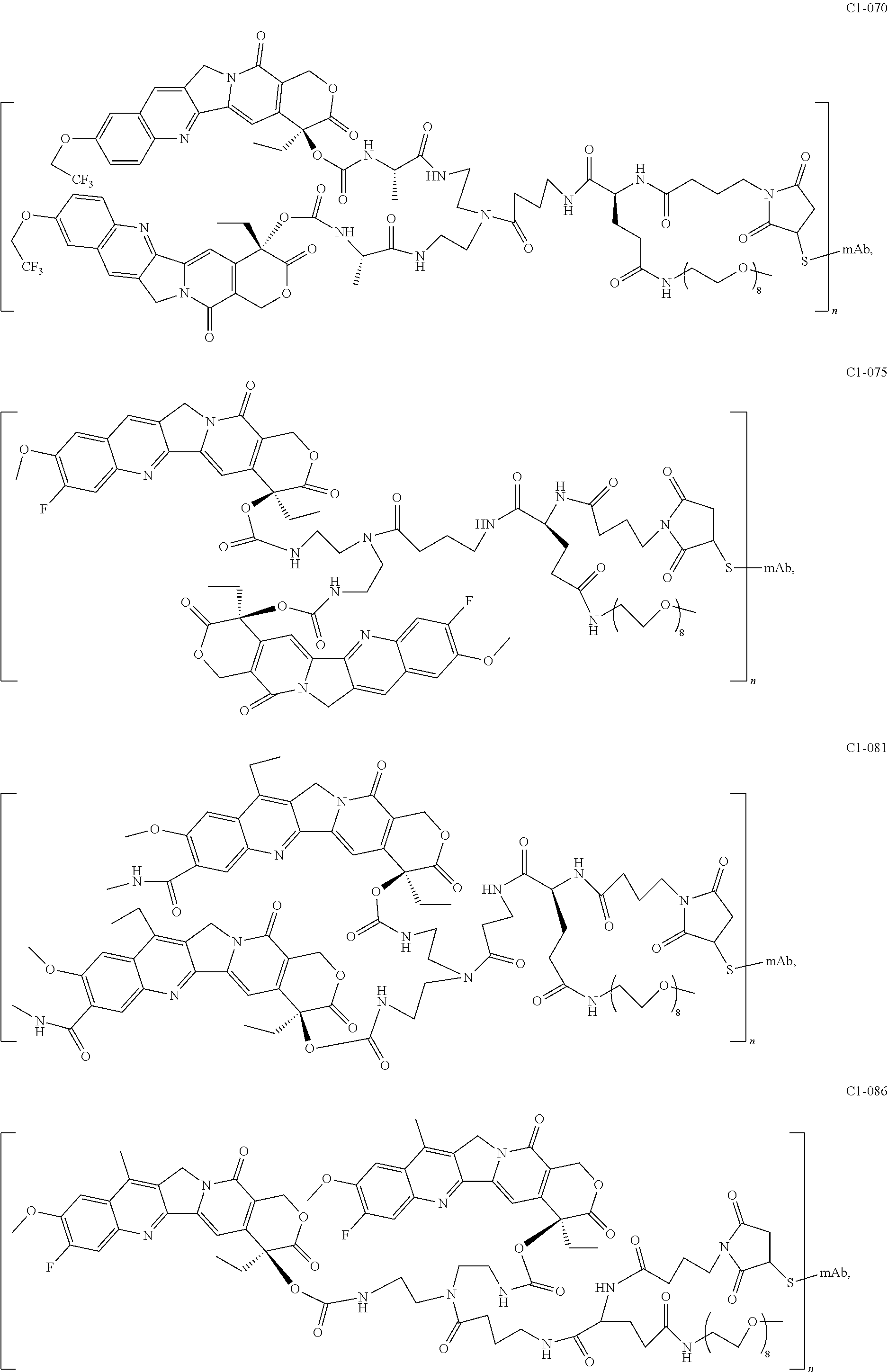 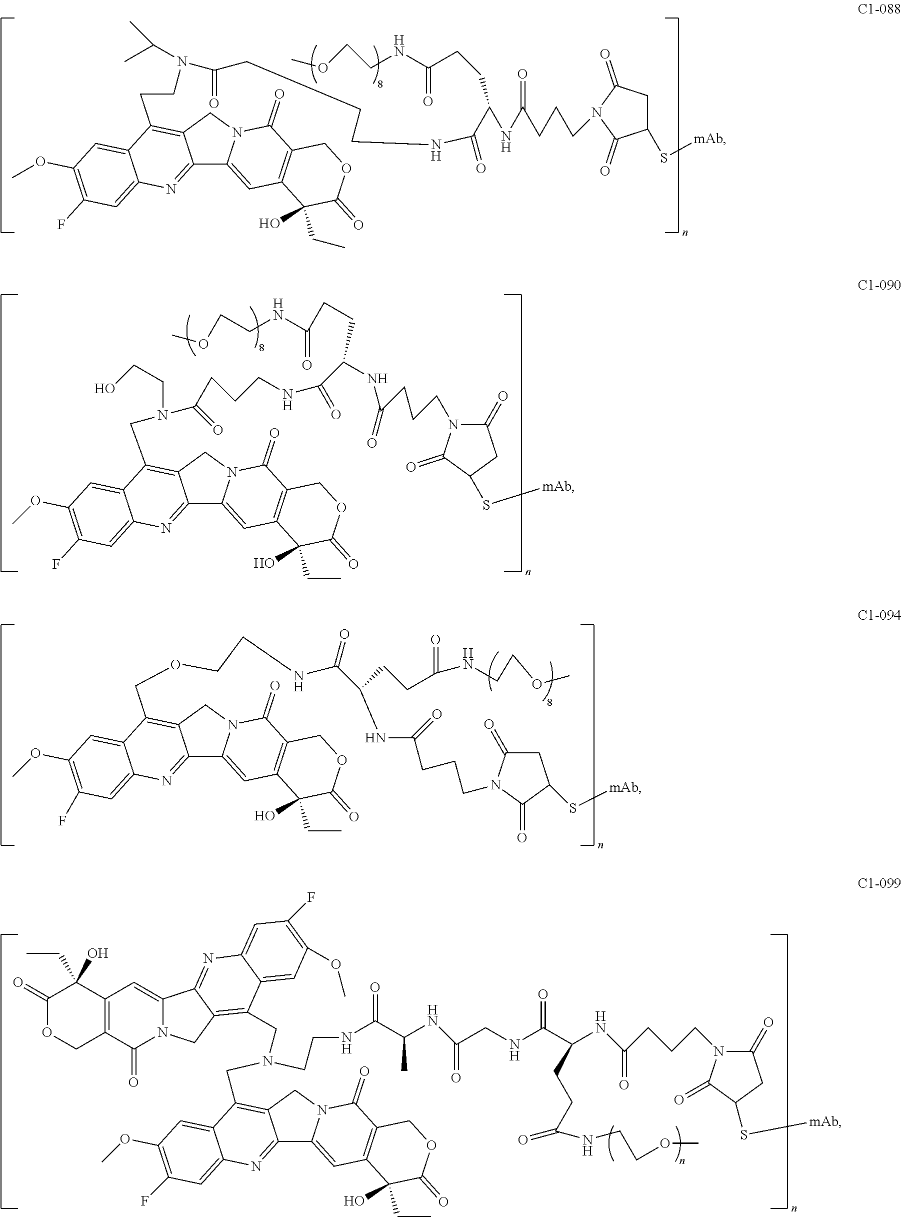 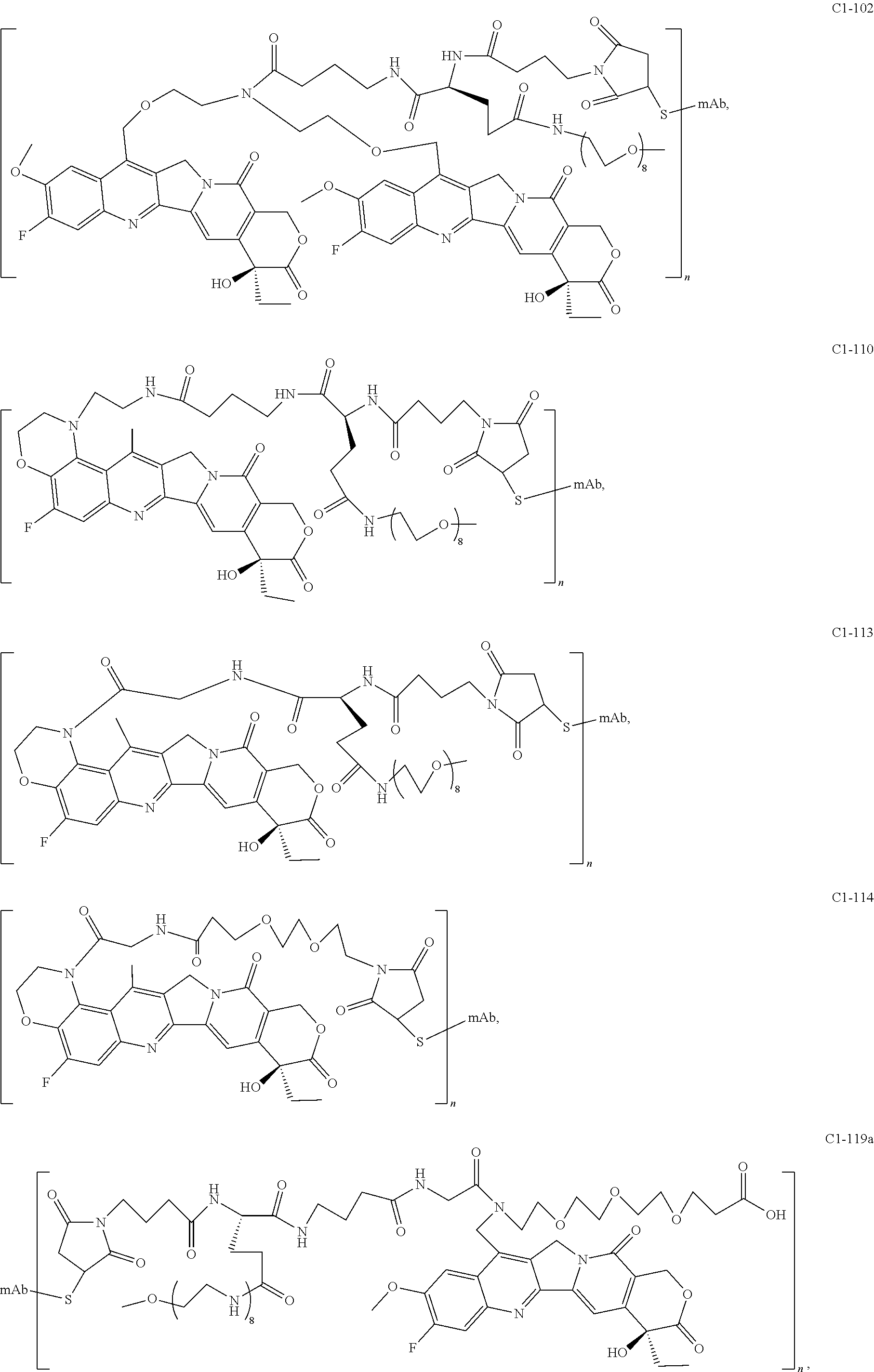 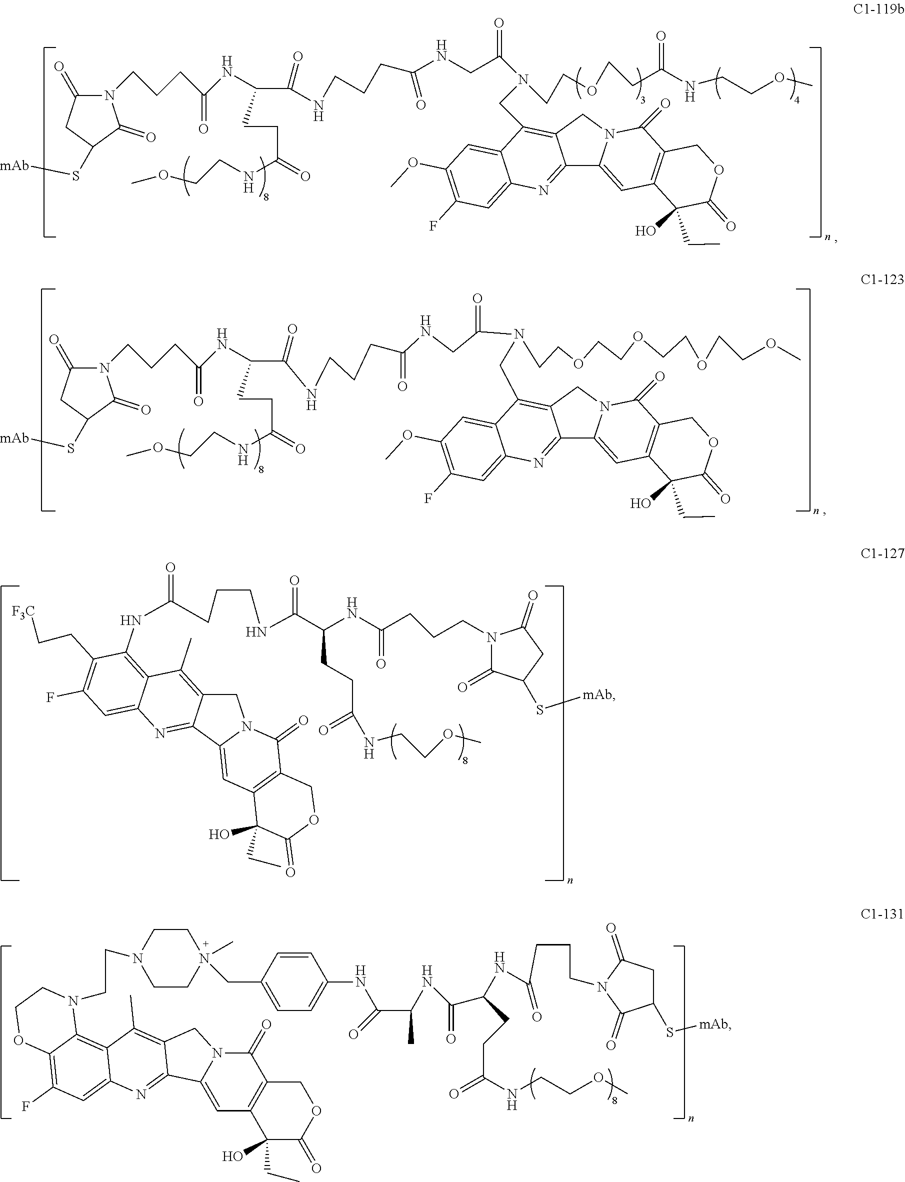 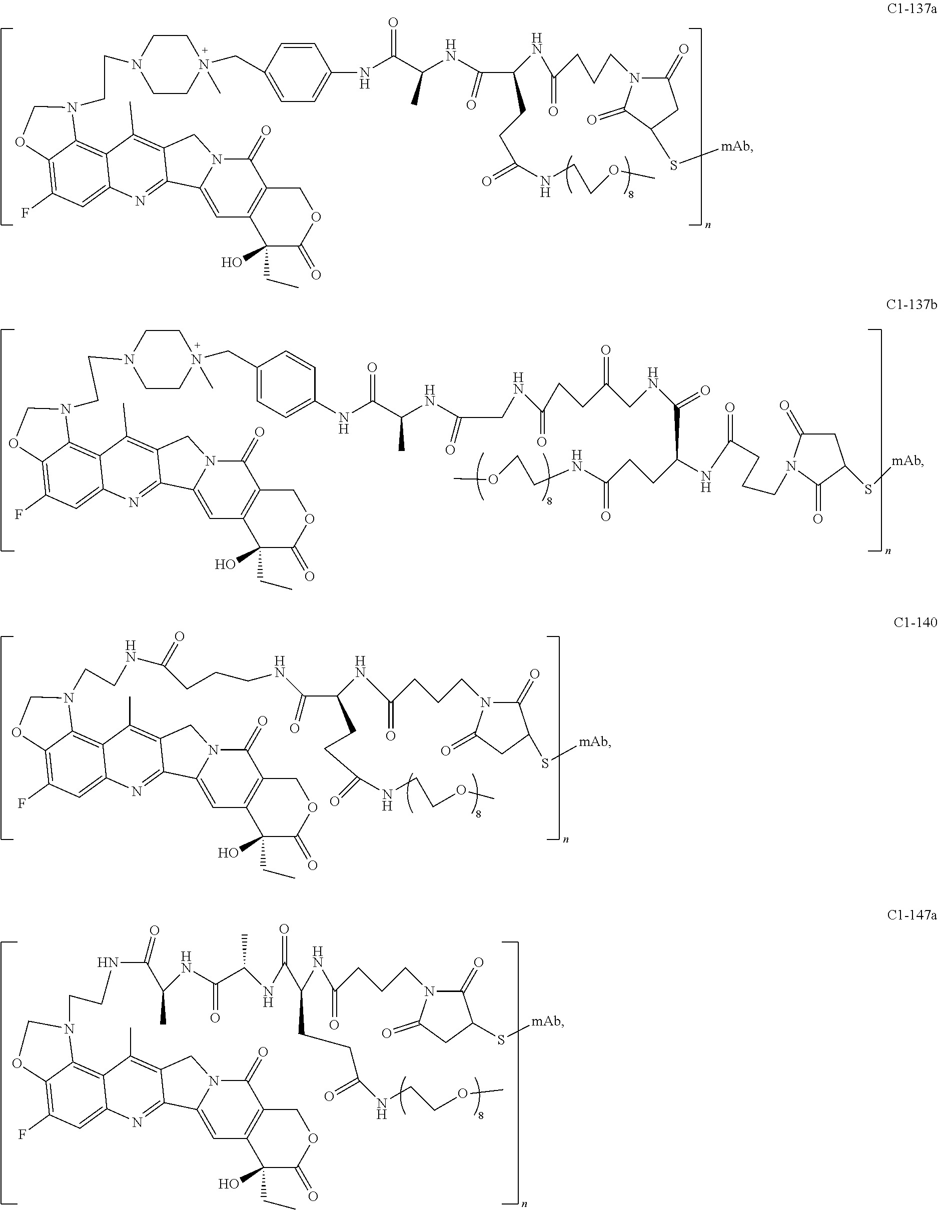 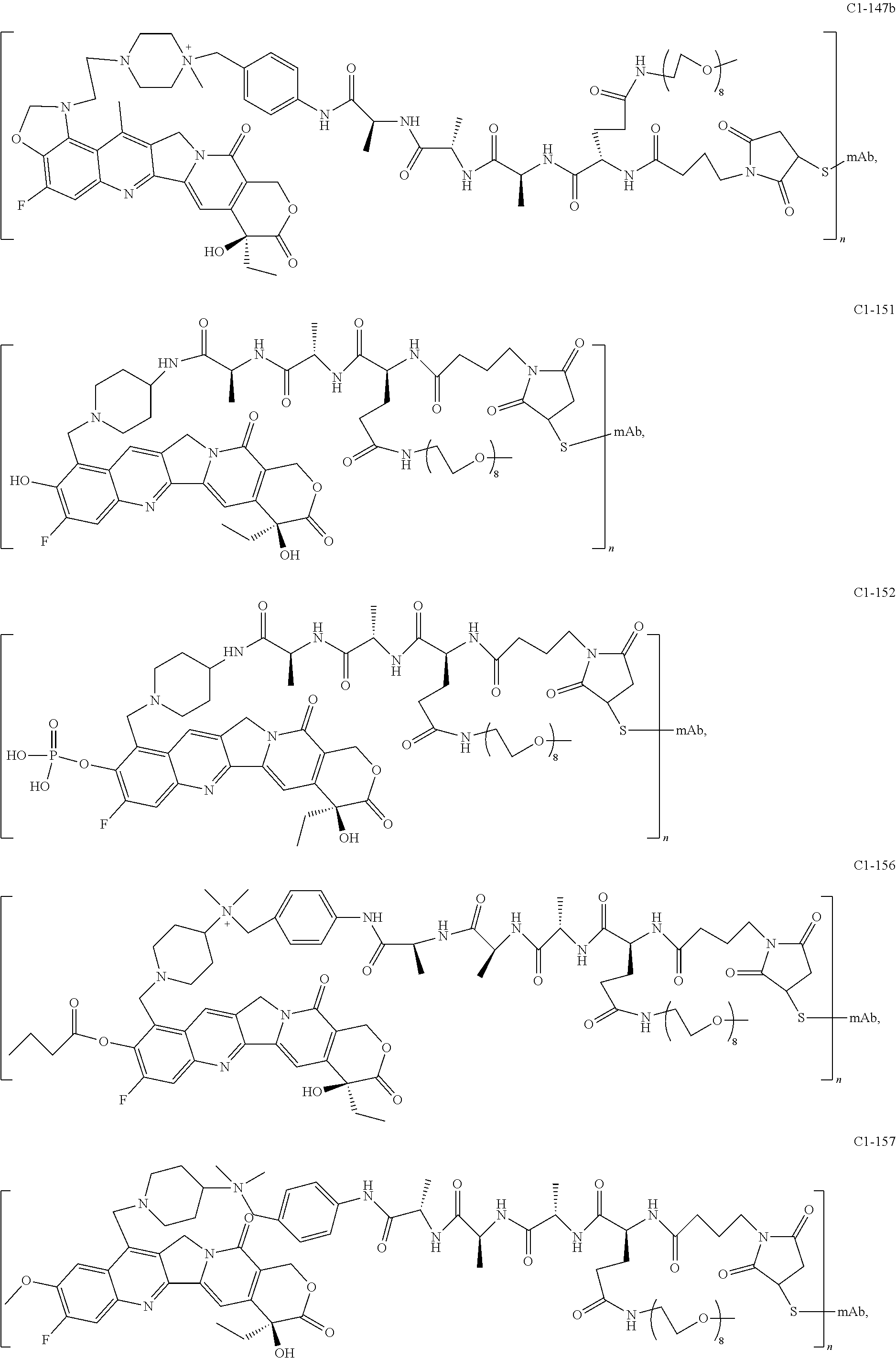 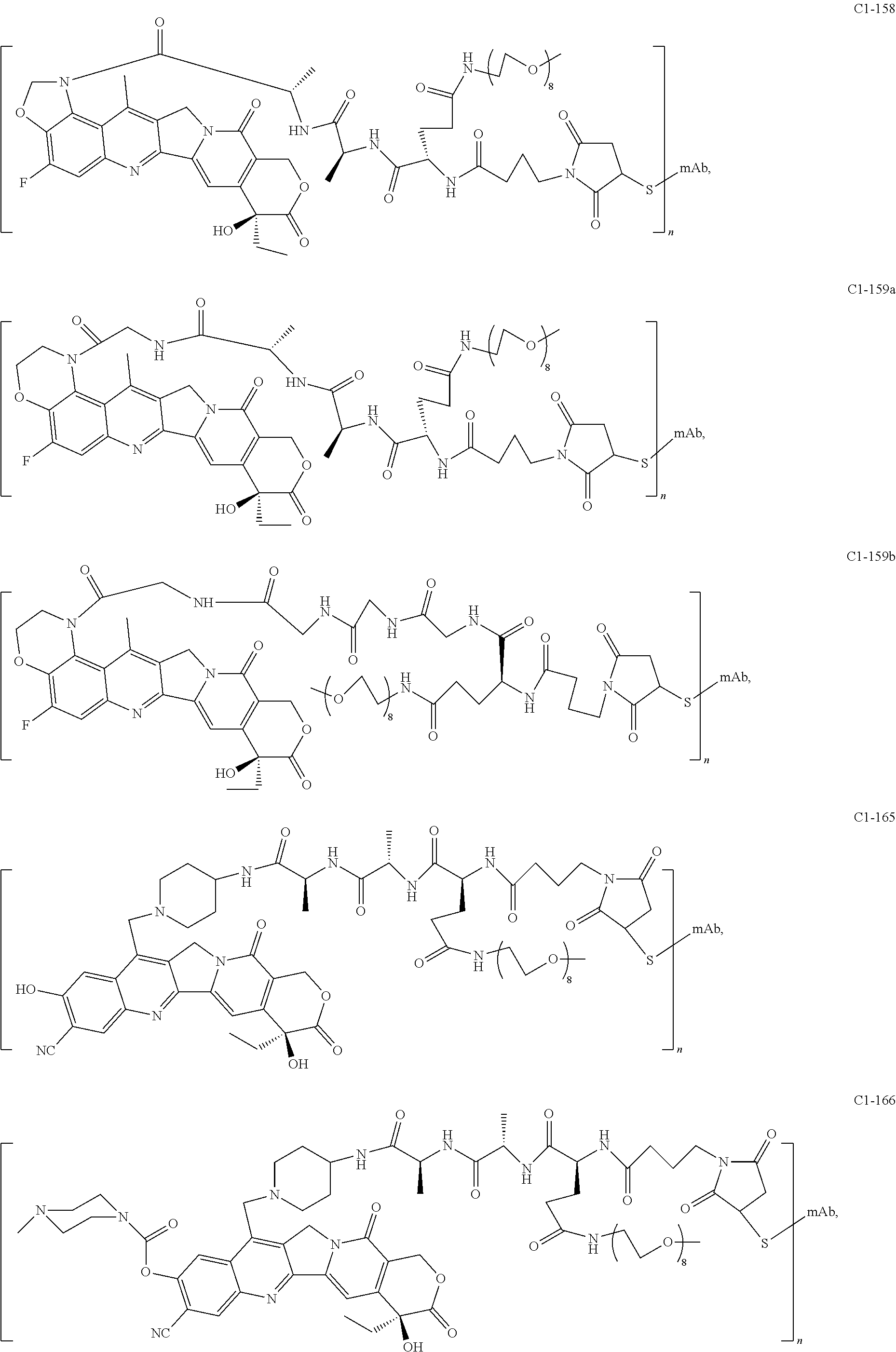 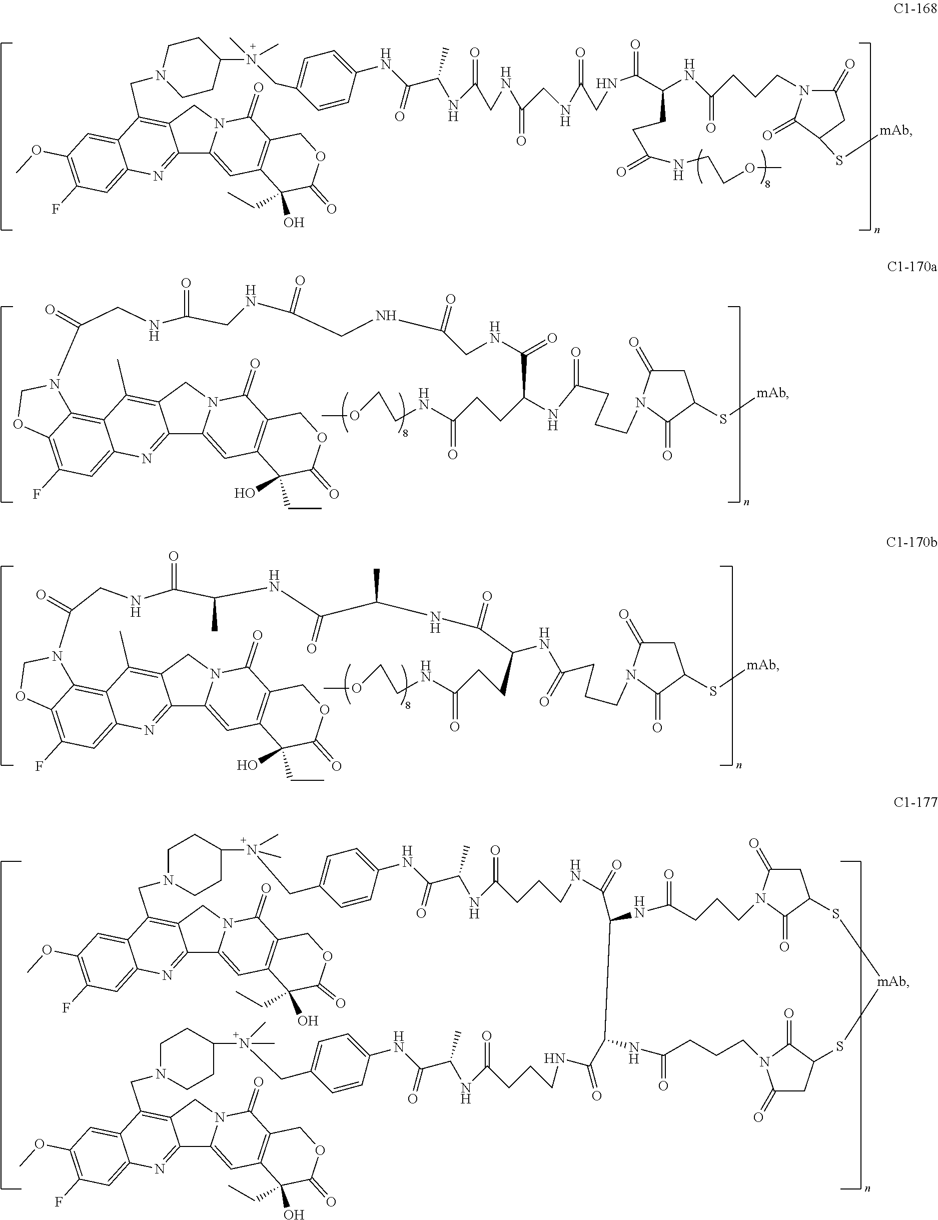 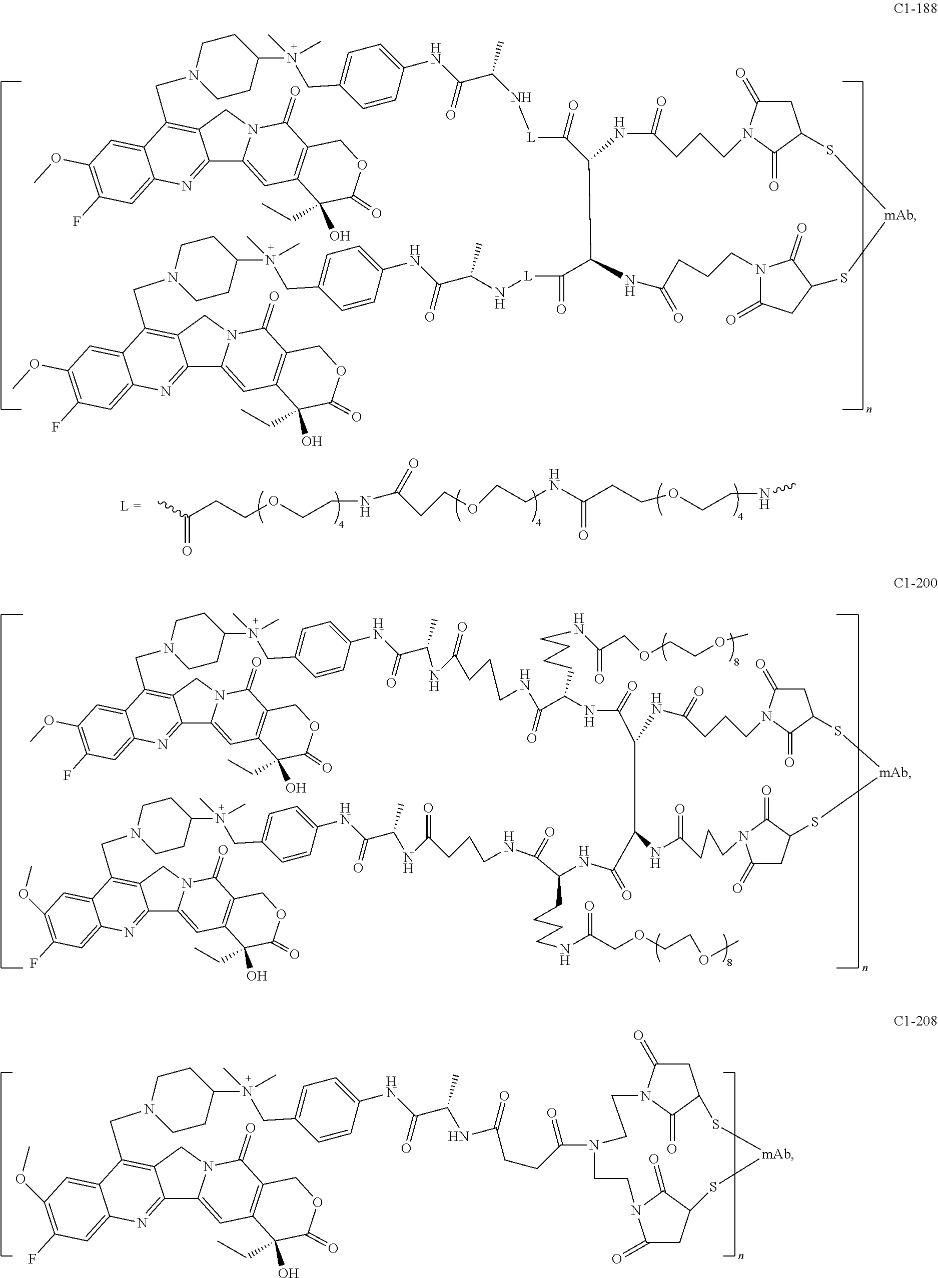 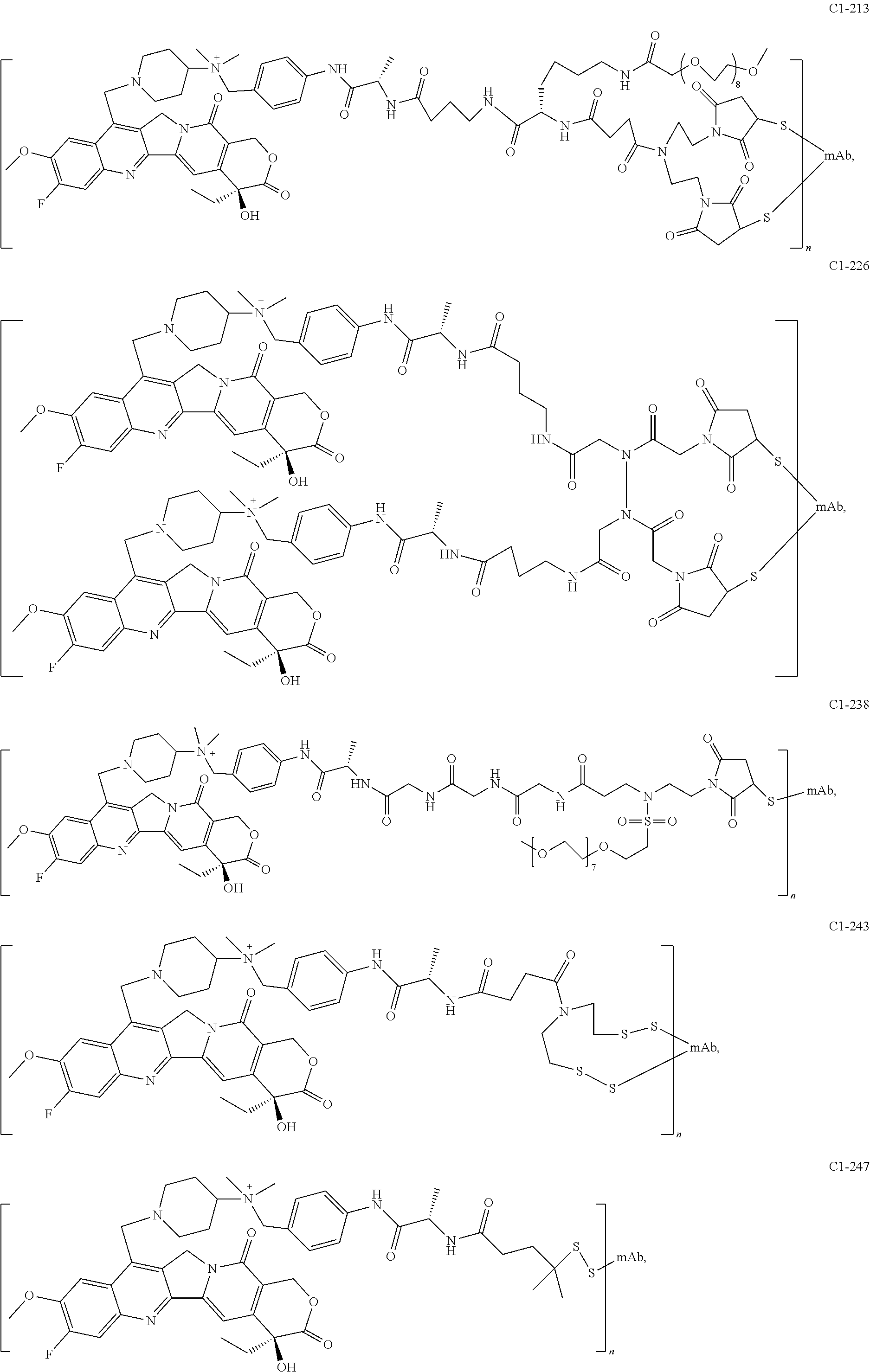 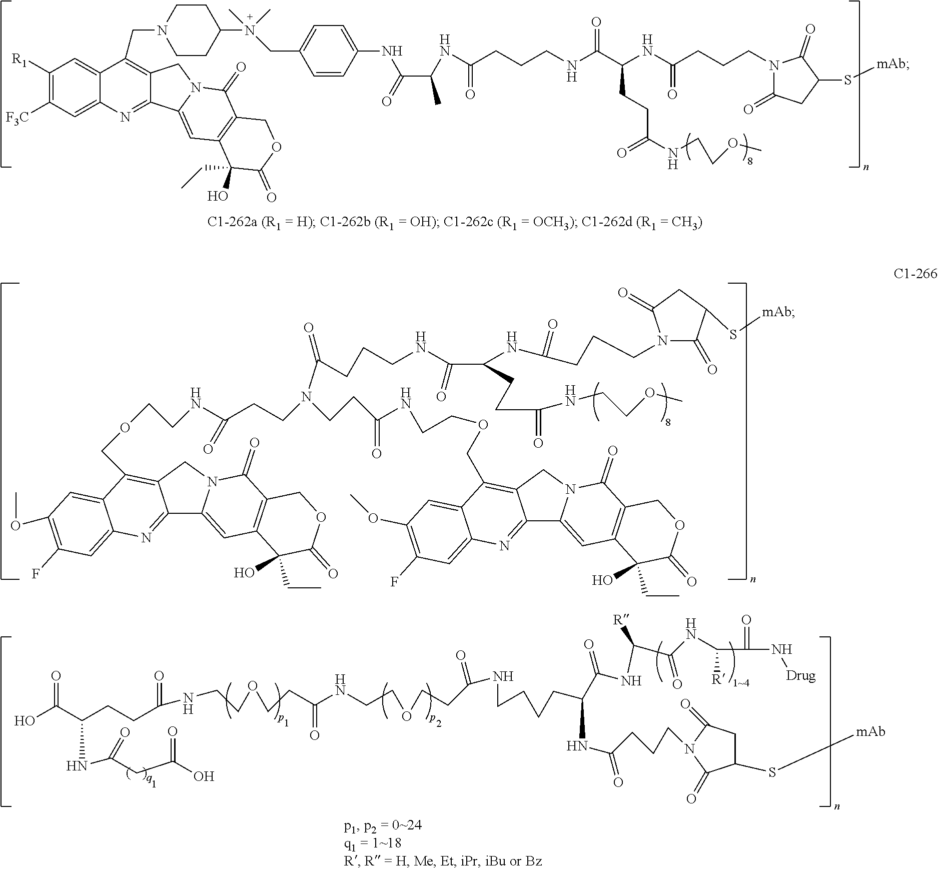
C1-285a˜C1285z, C1-285a₁-C1285i₁, wherein 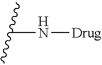
are 285a˜285z, 285a₁˜285i₁ illustrated below: 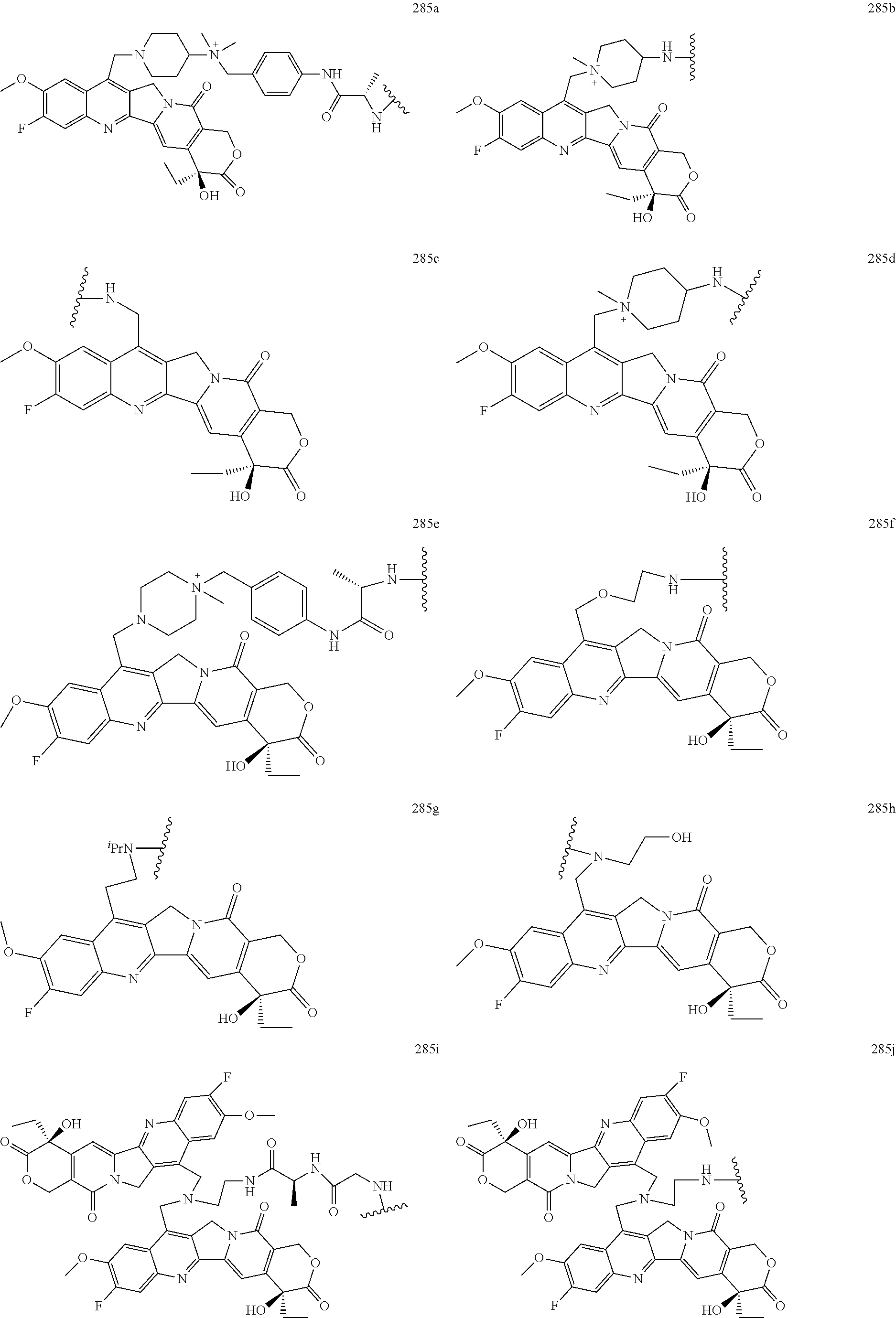 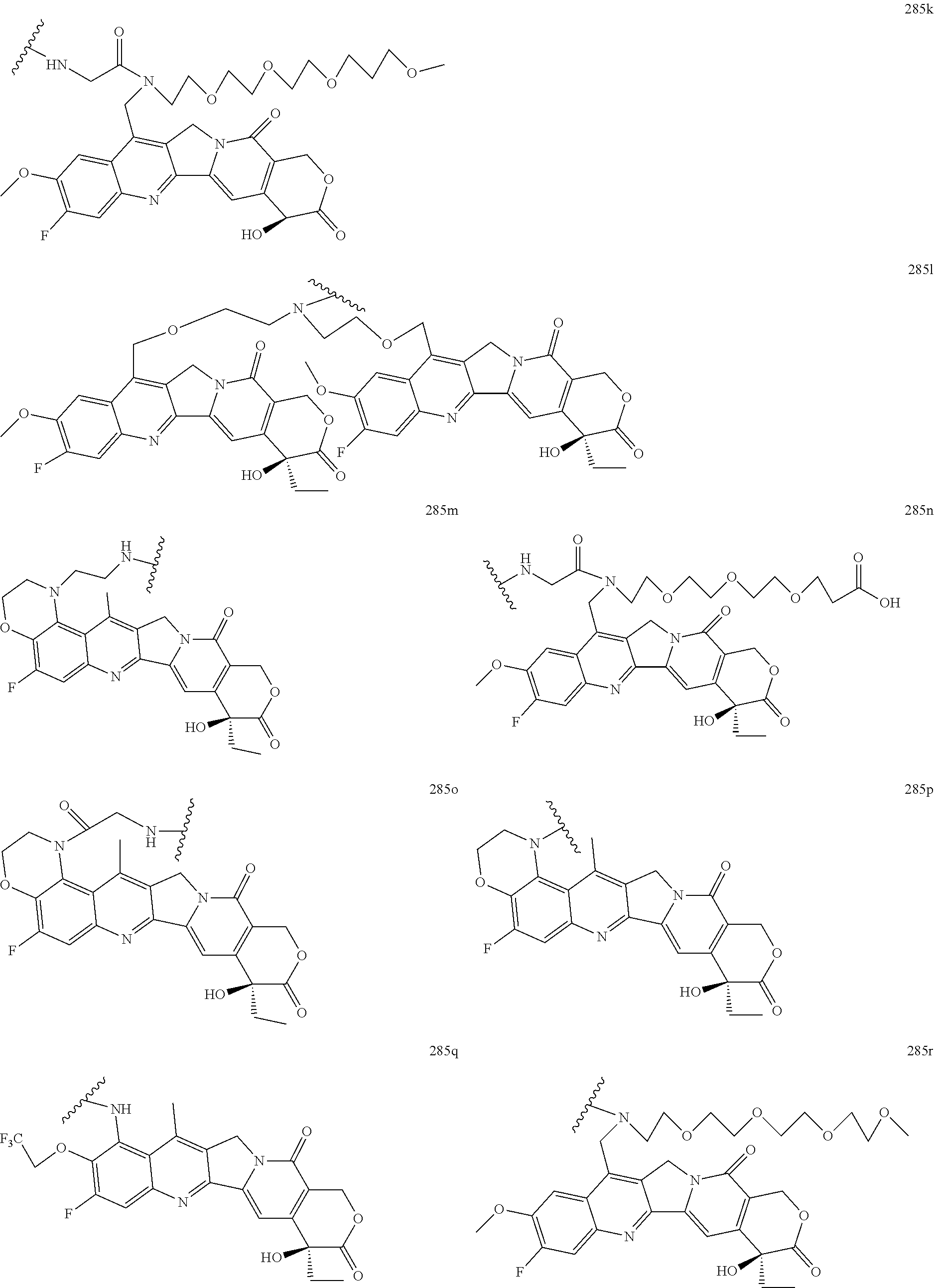 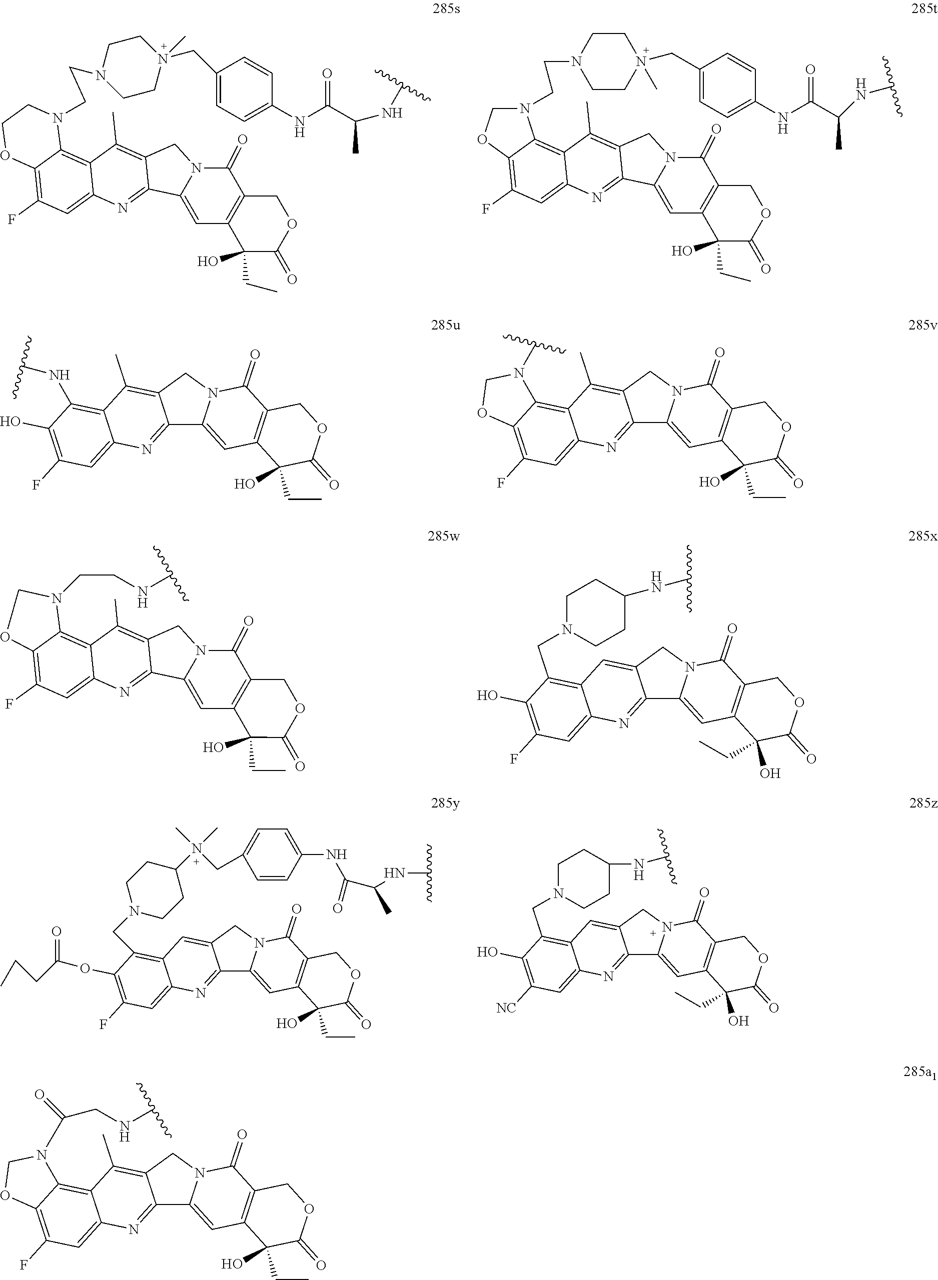 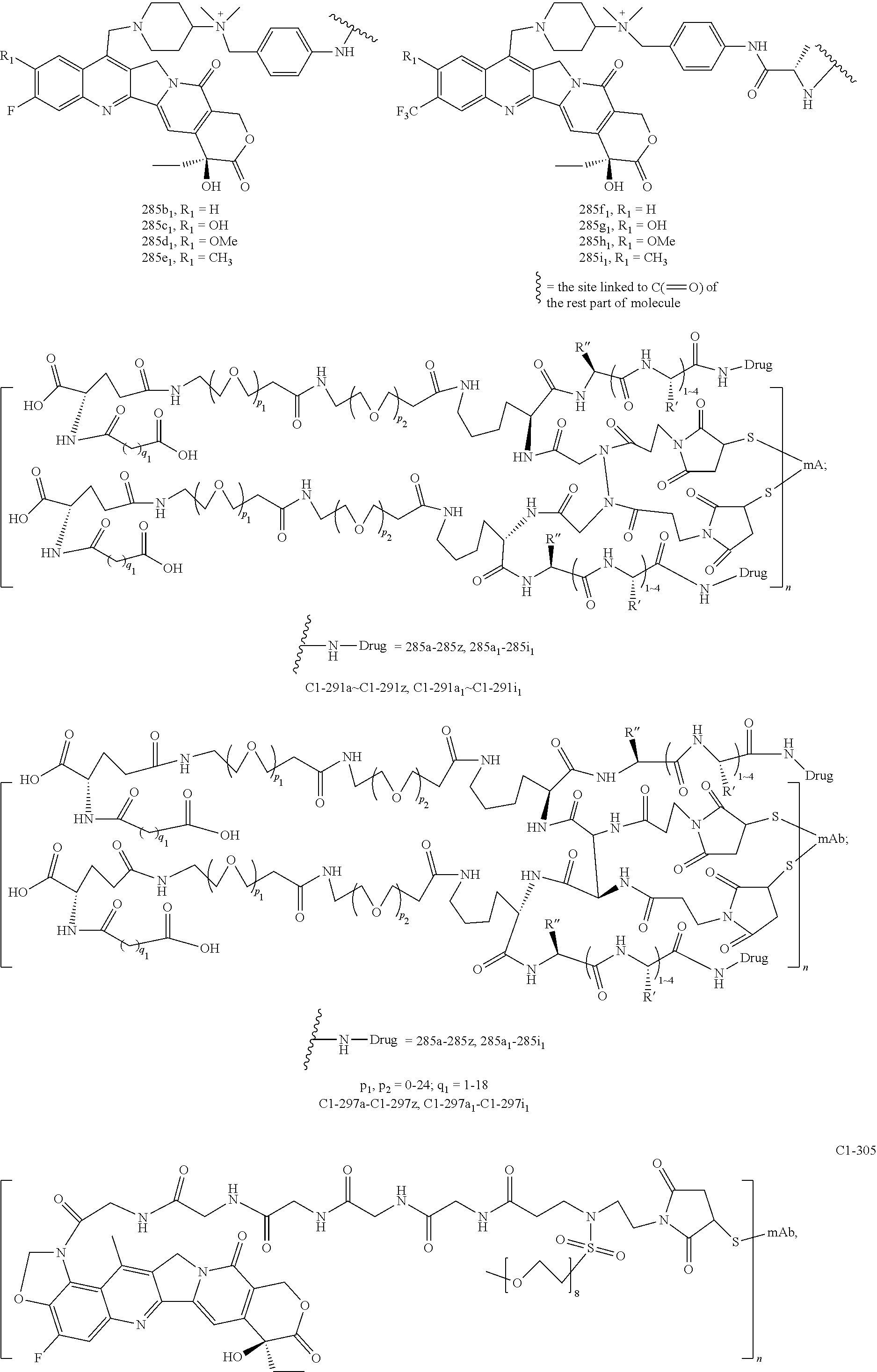 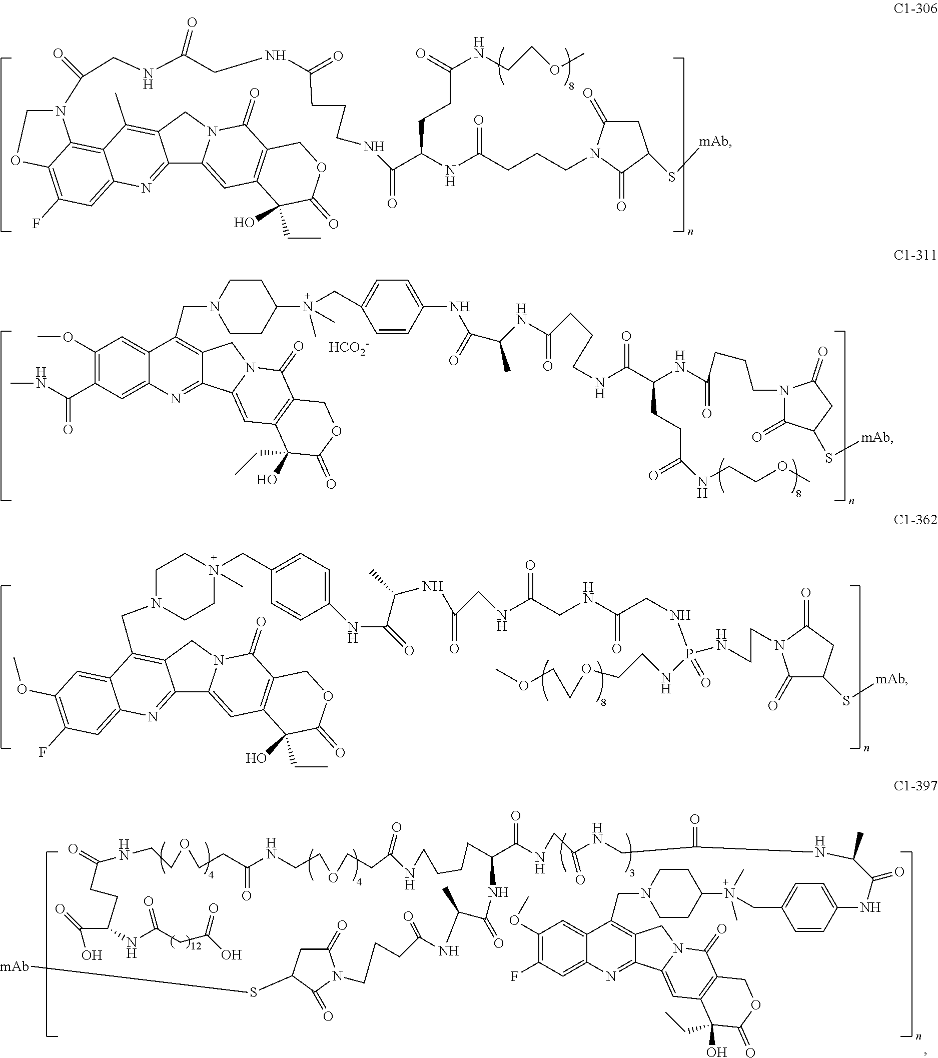 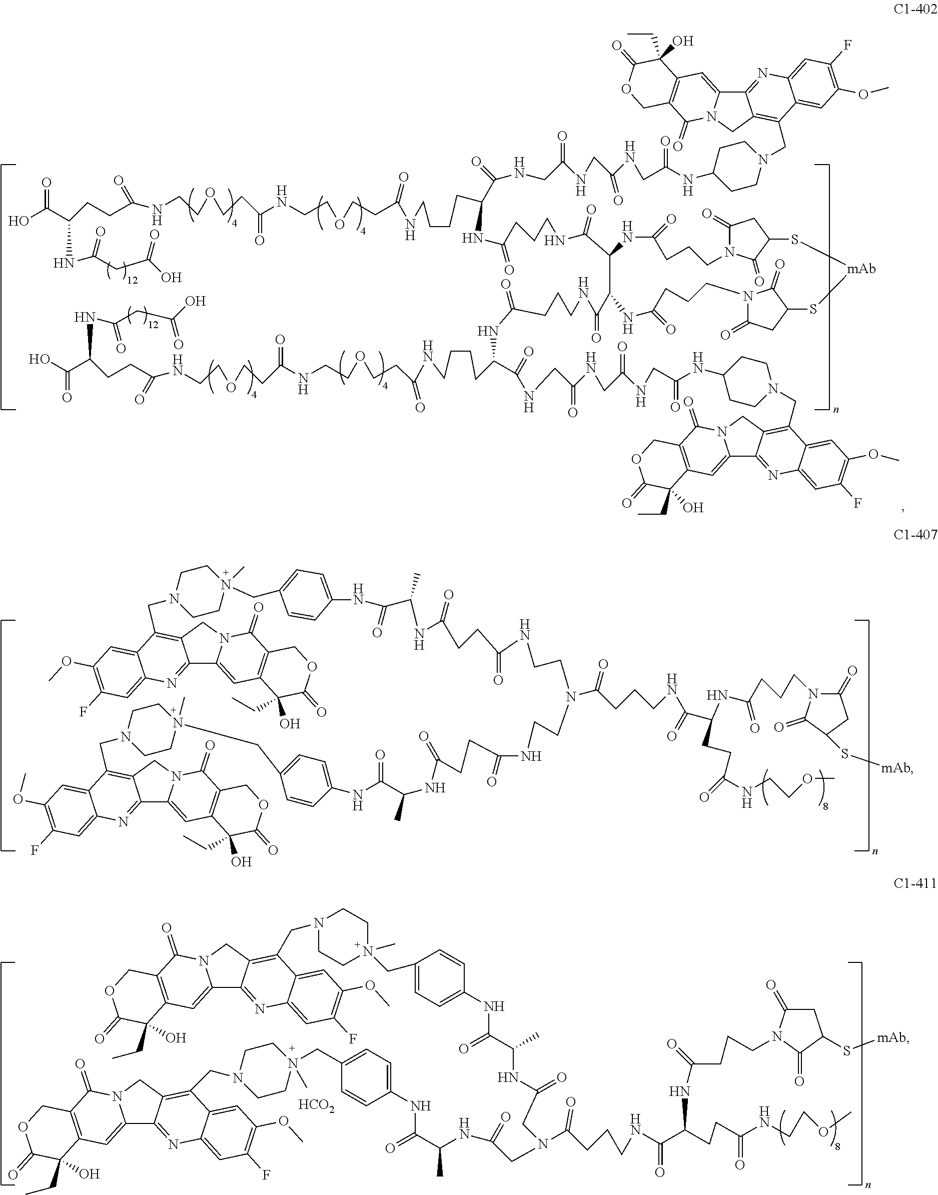 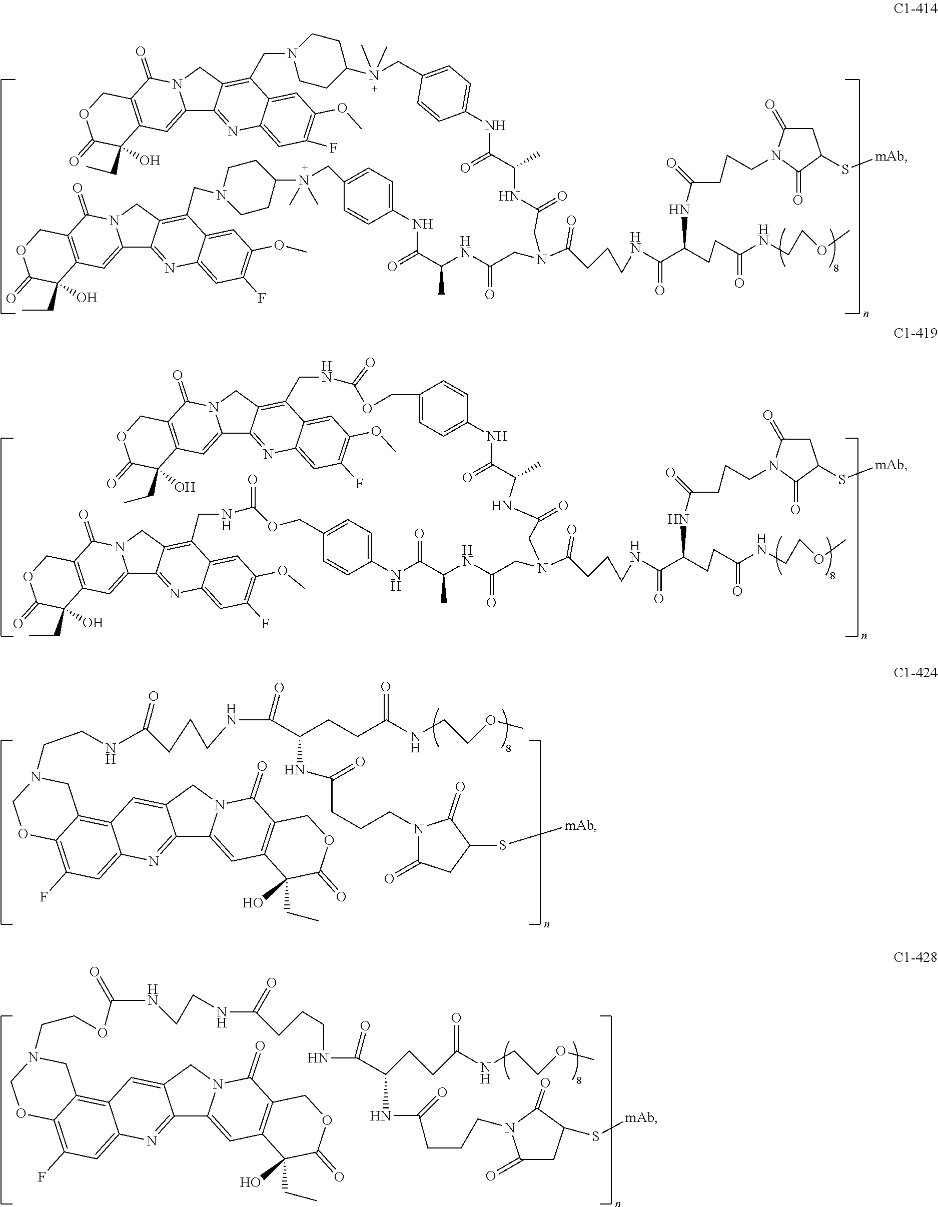 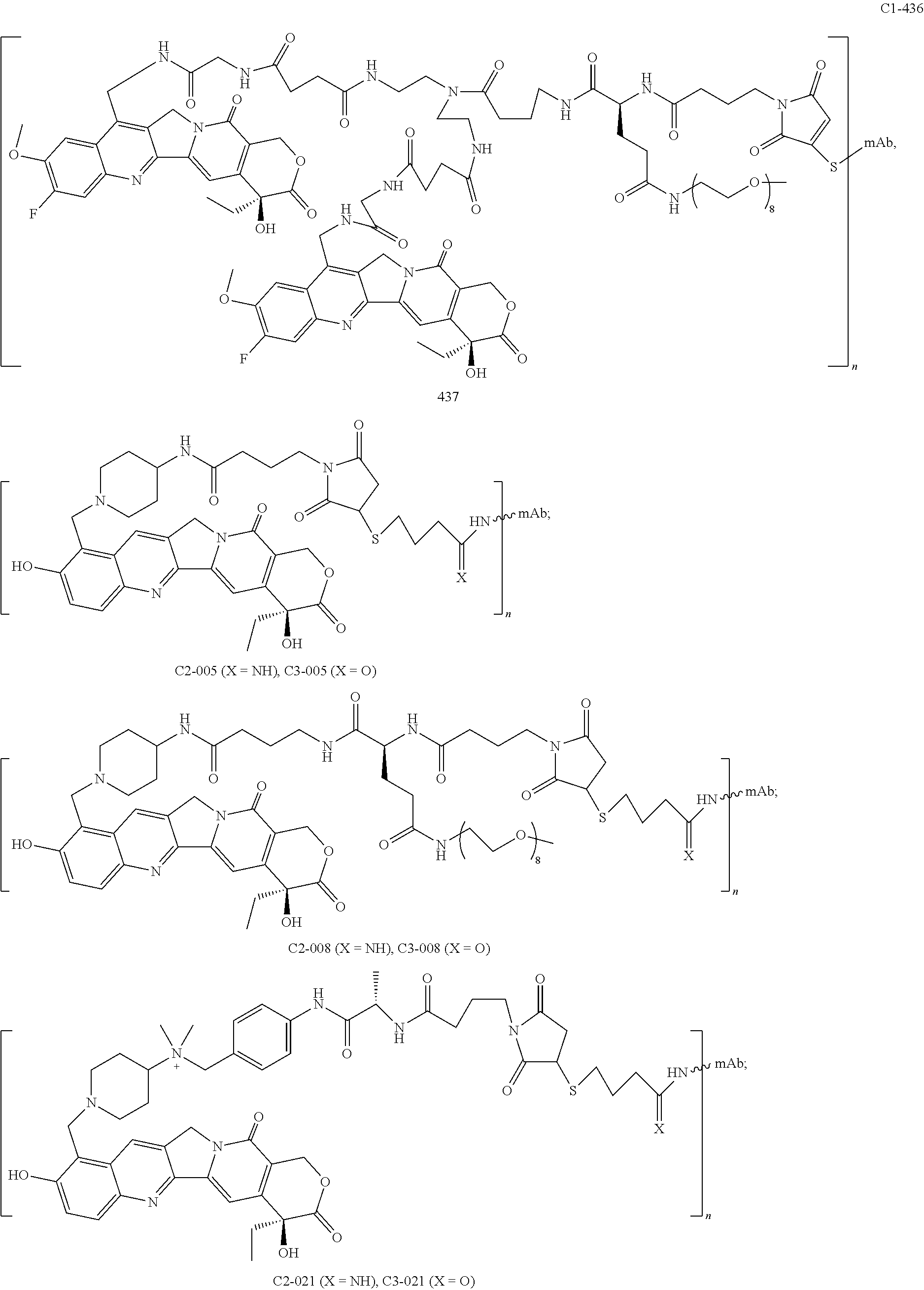 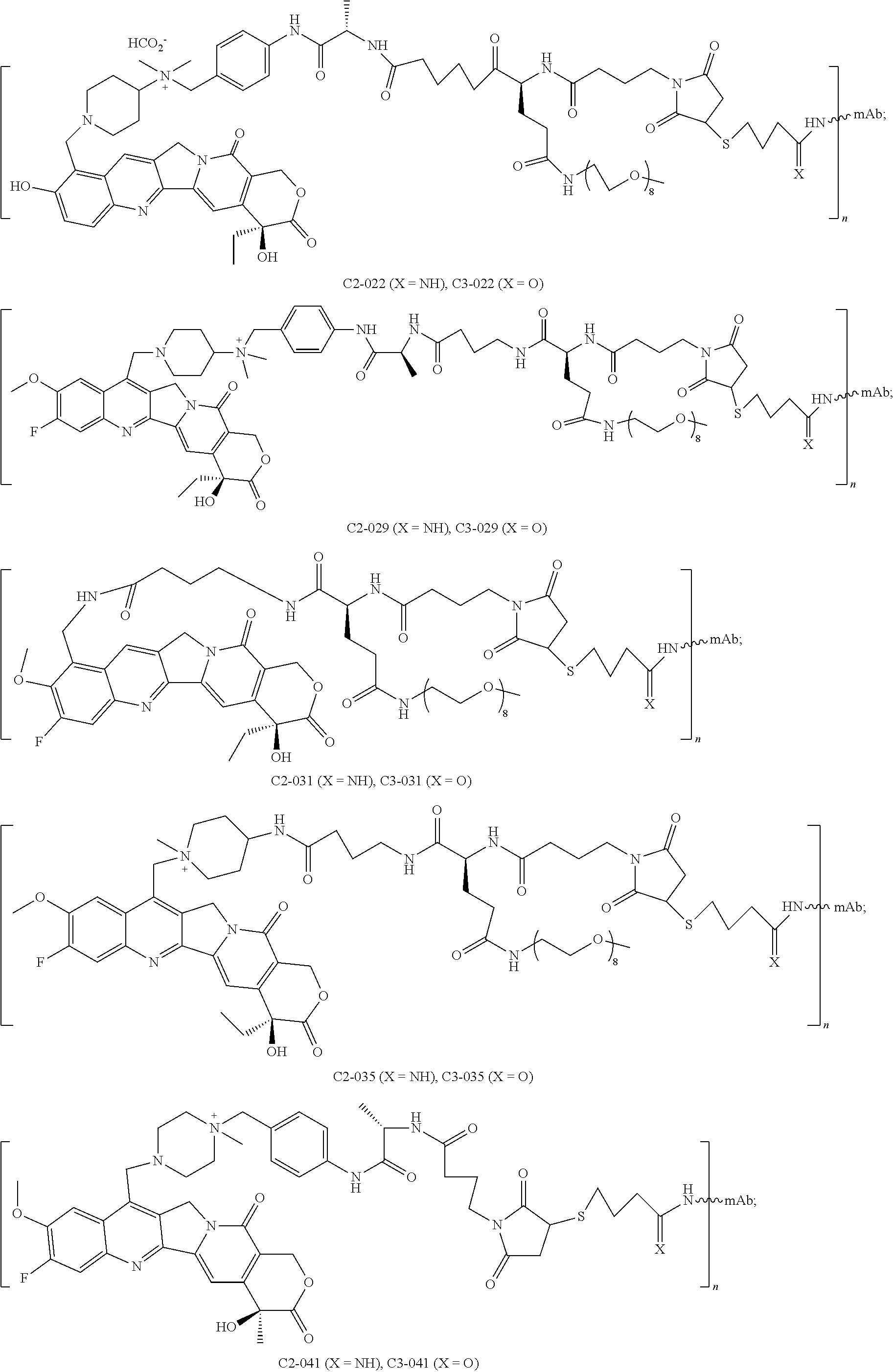 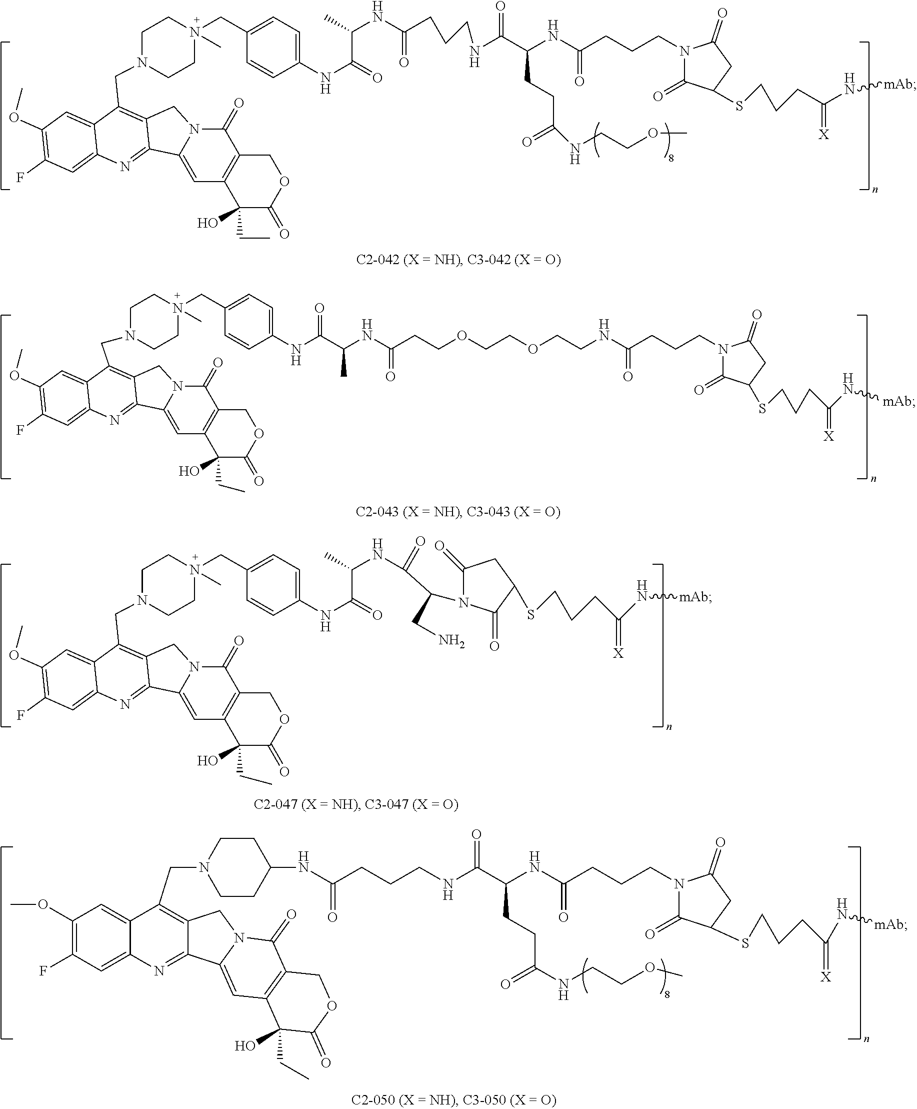 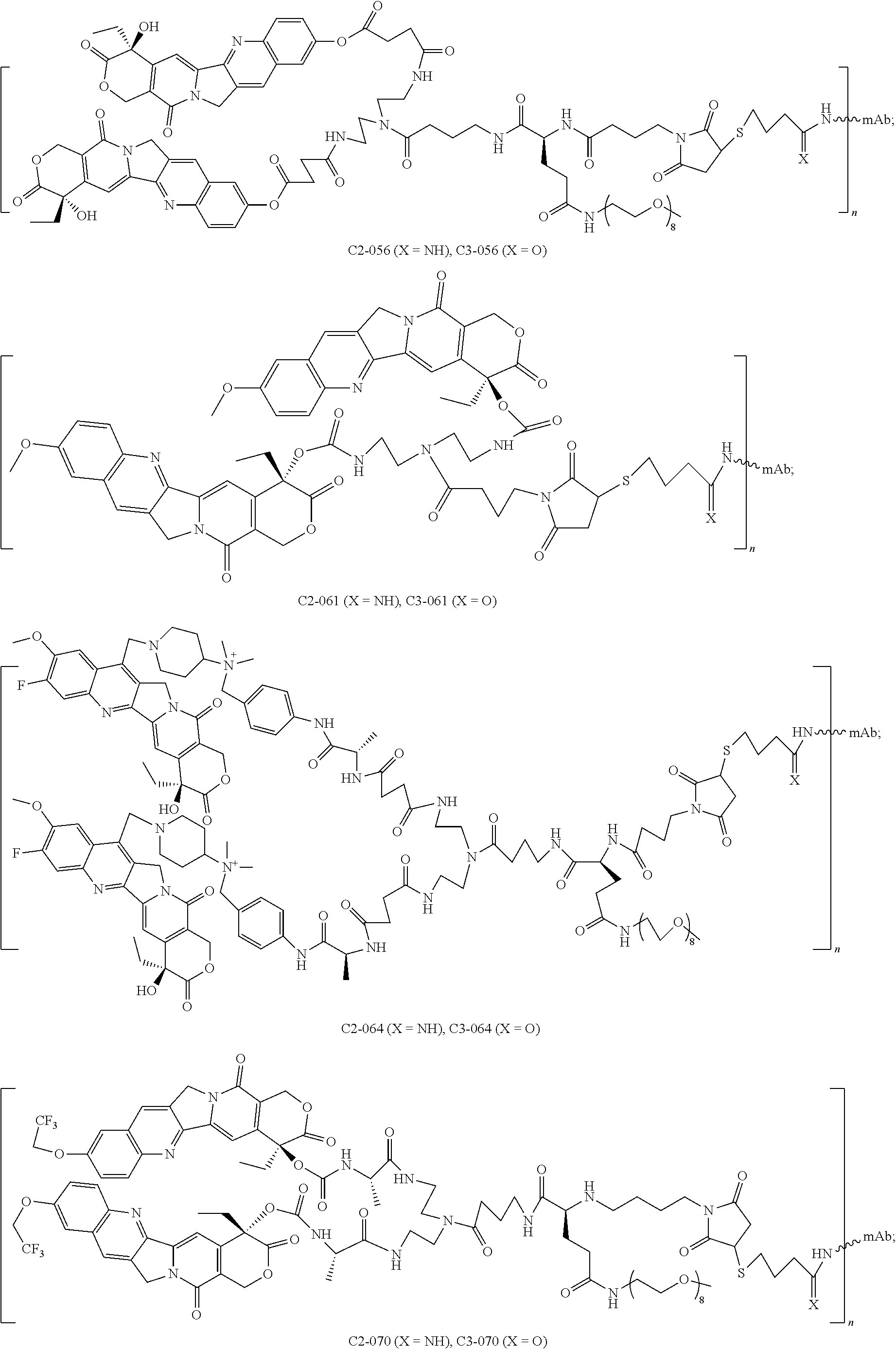 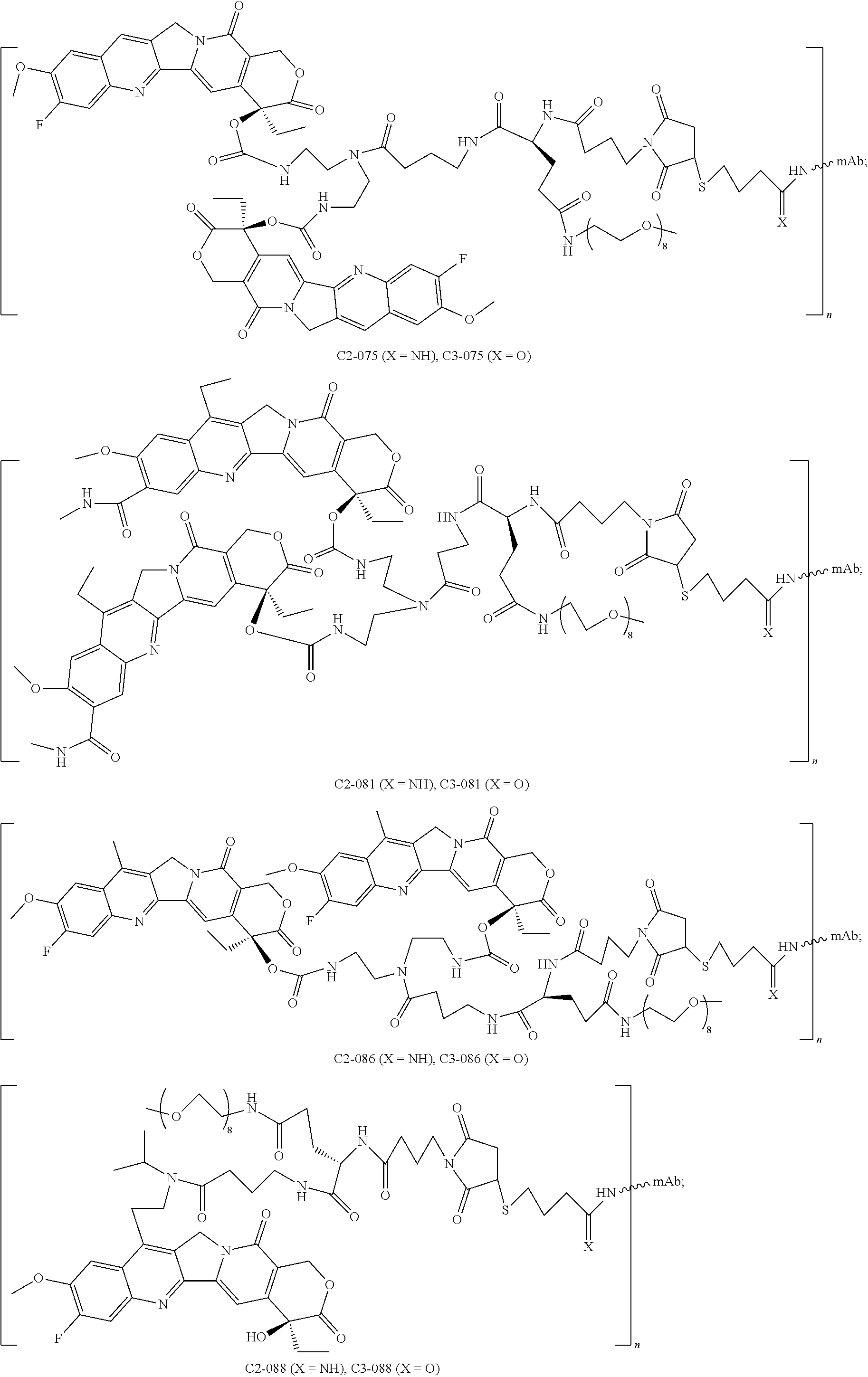 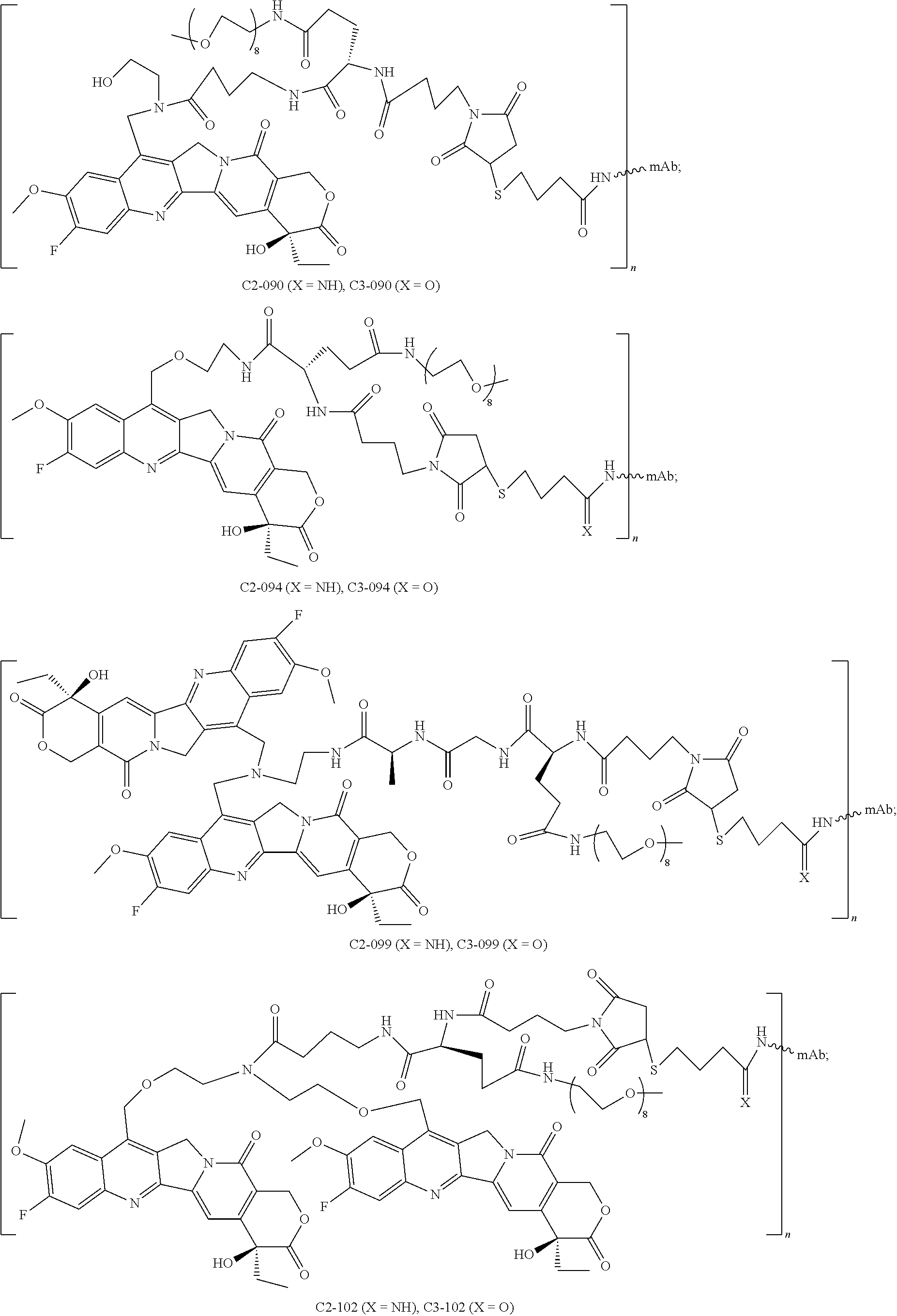 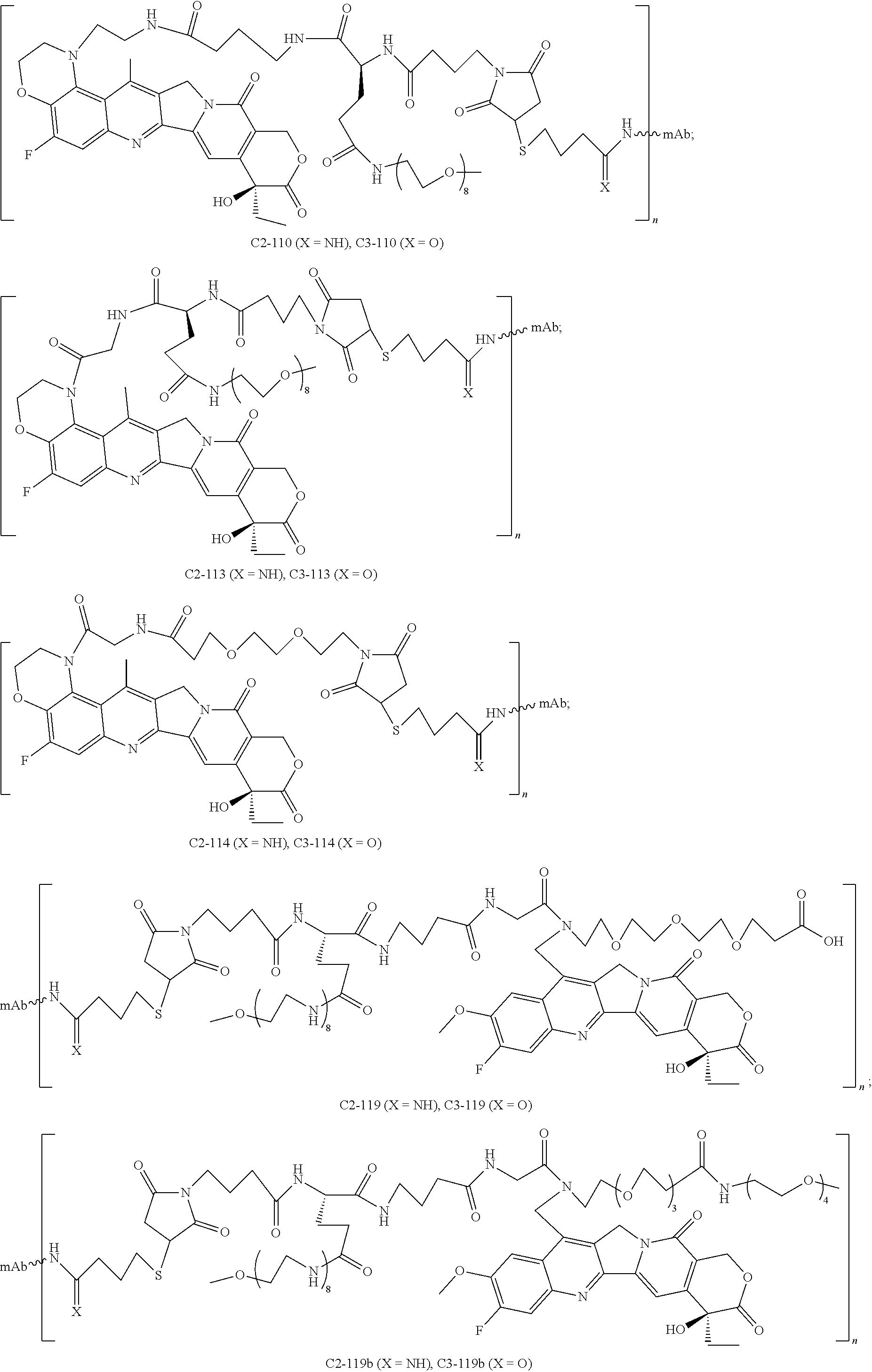 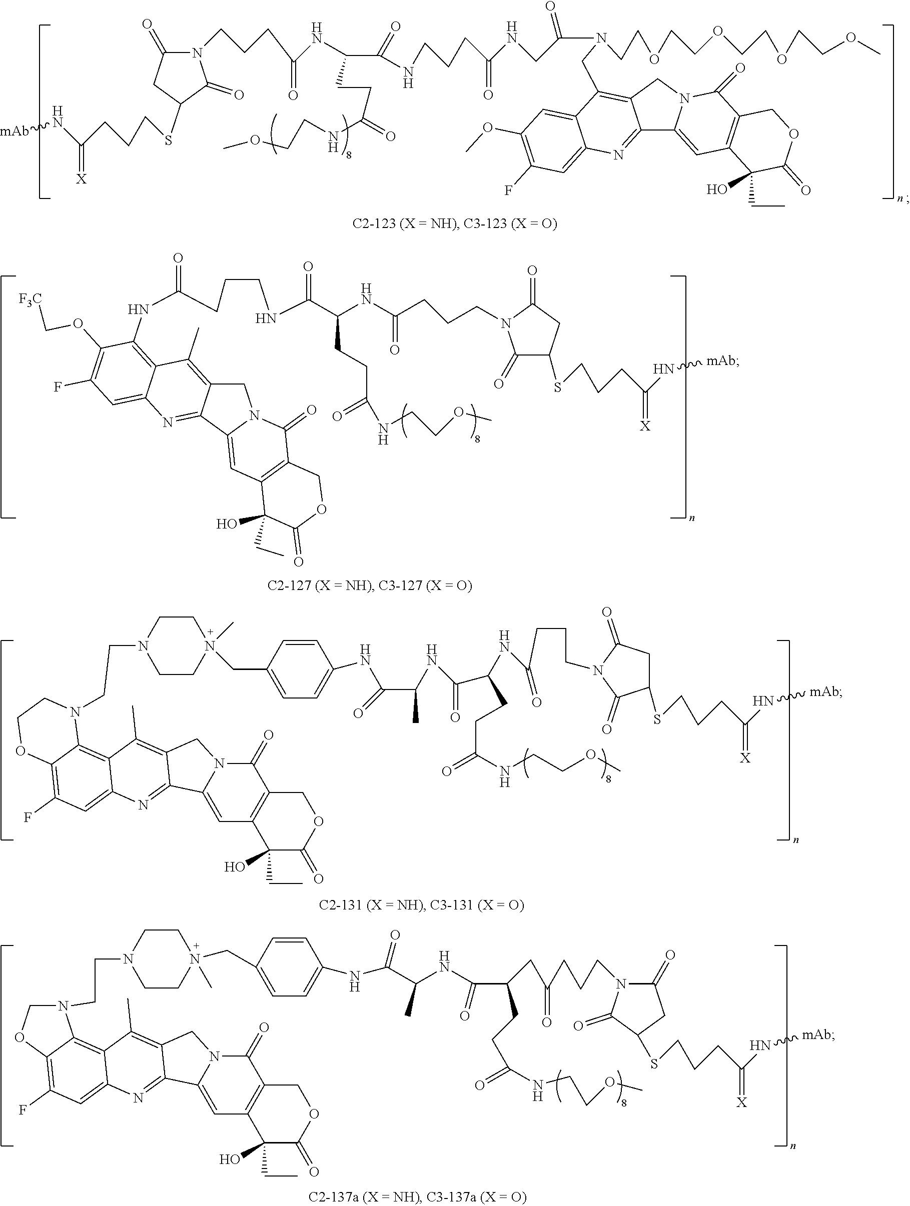 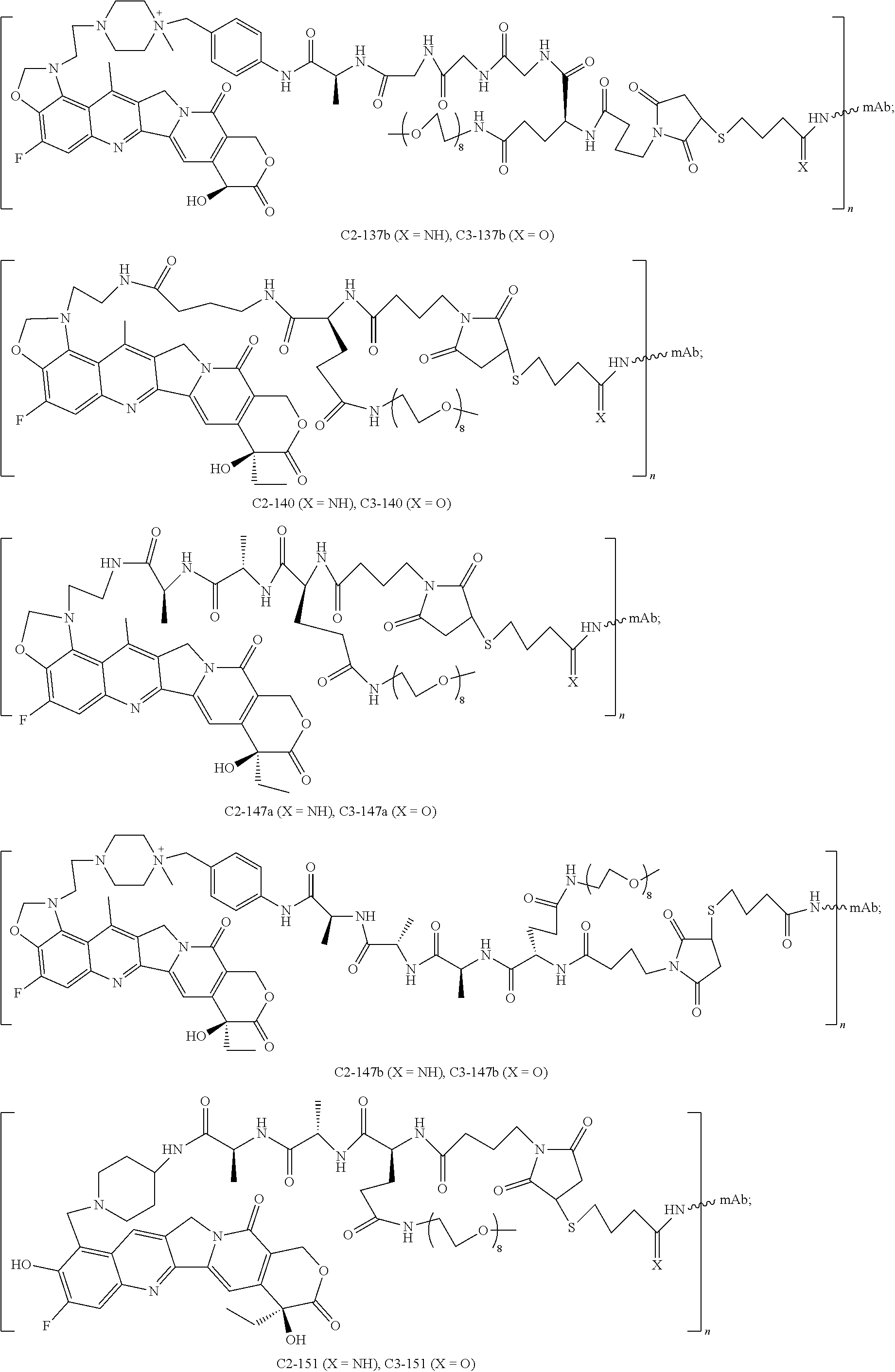 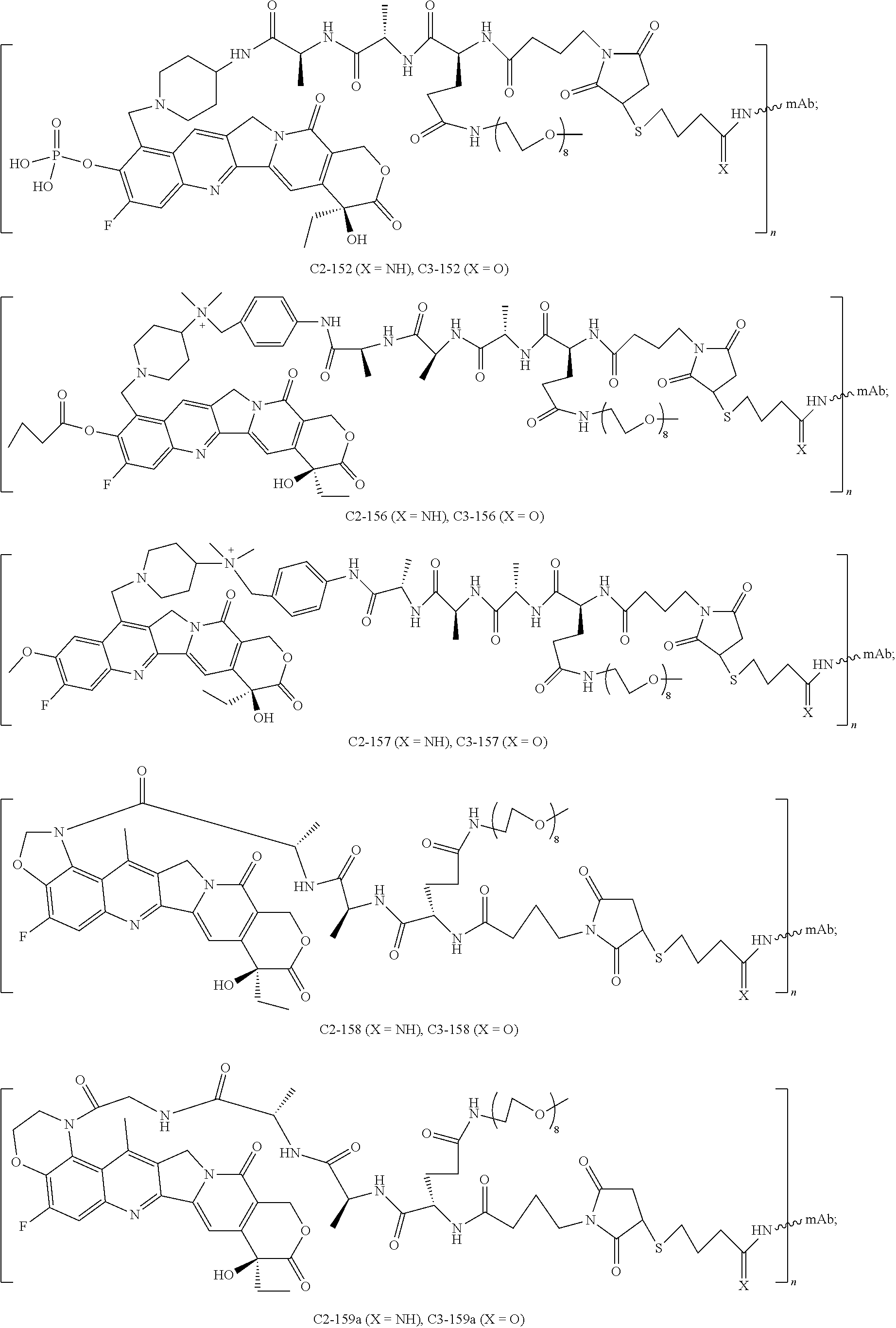 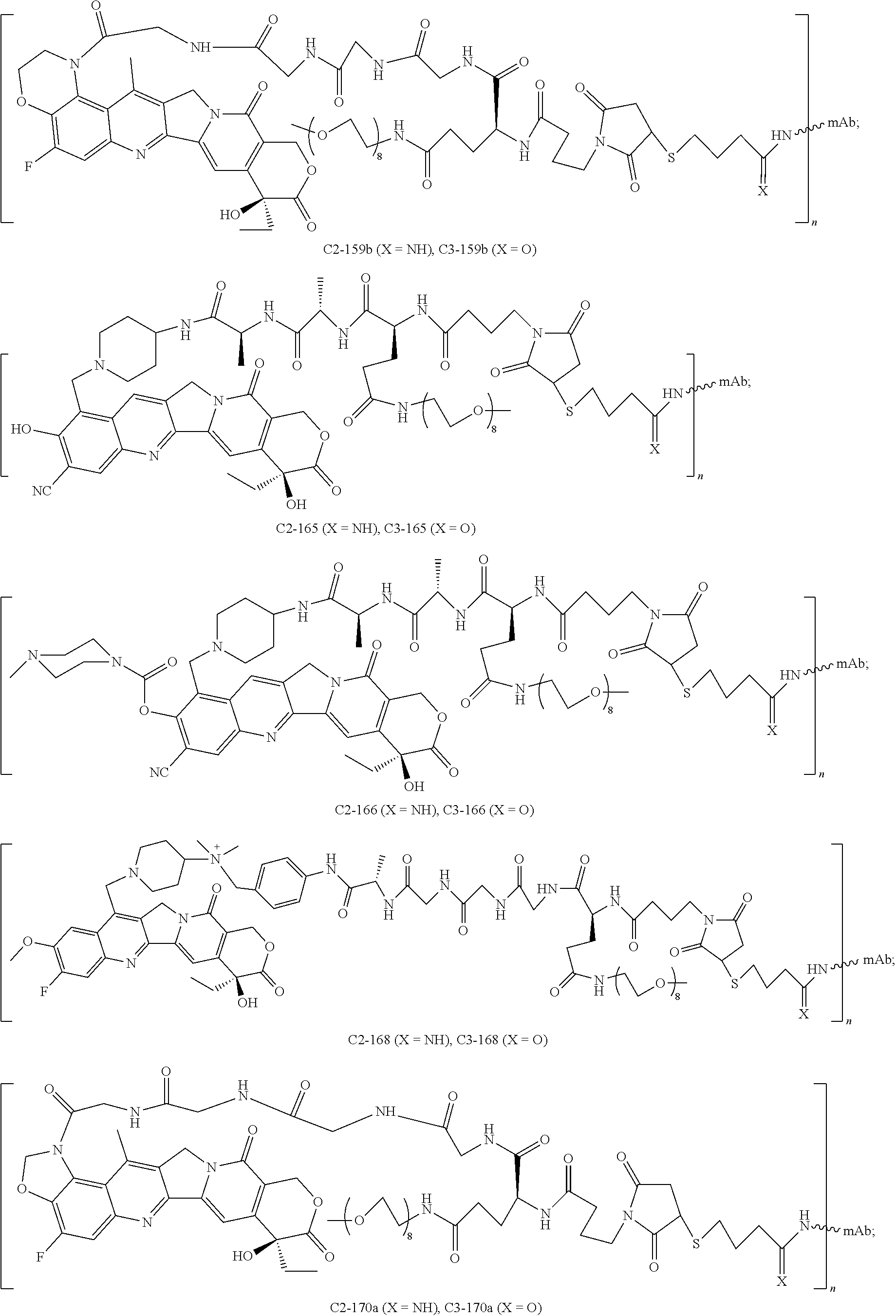 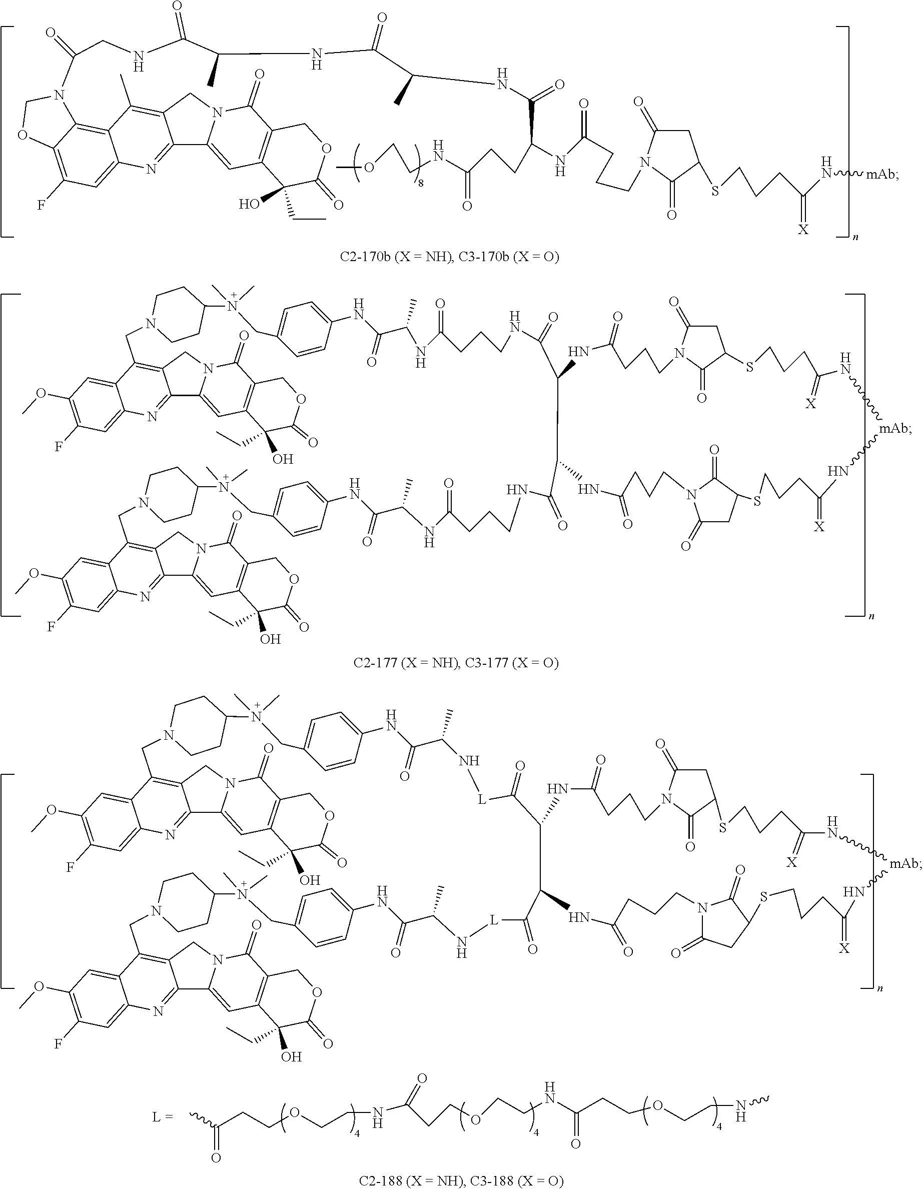 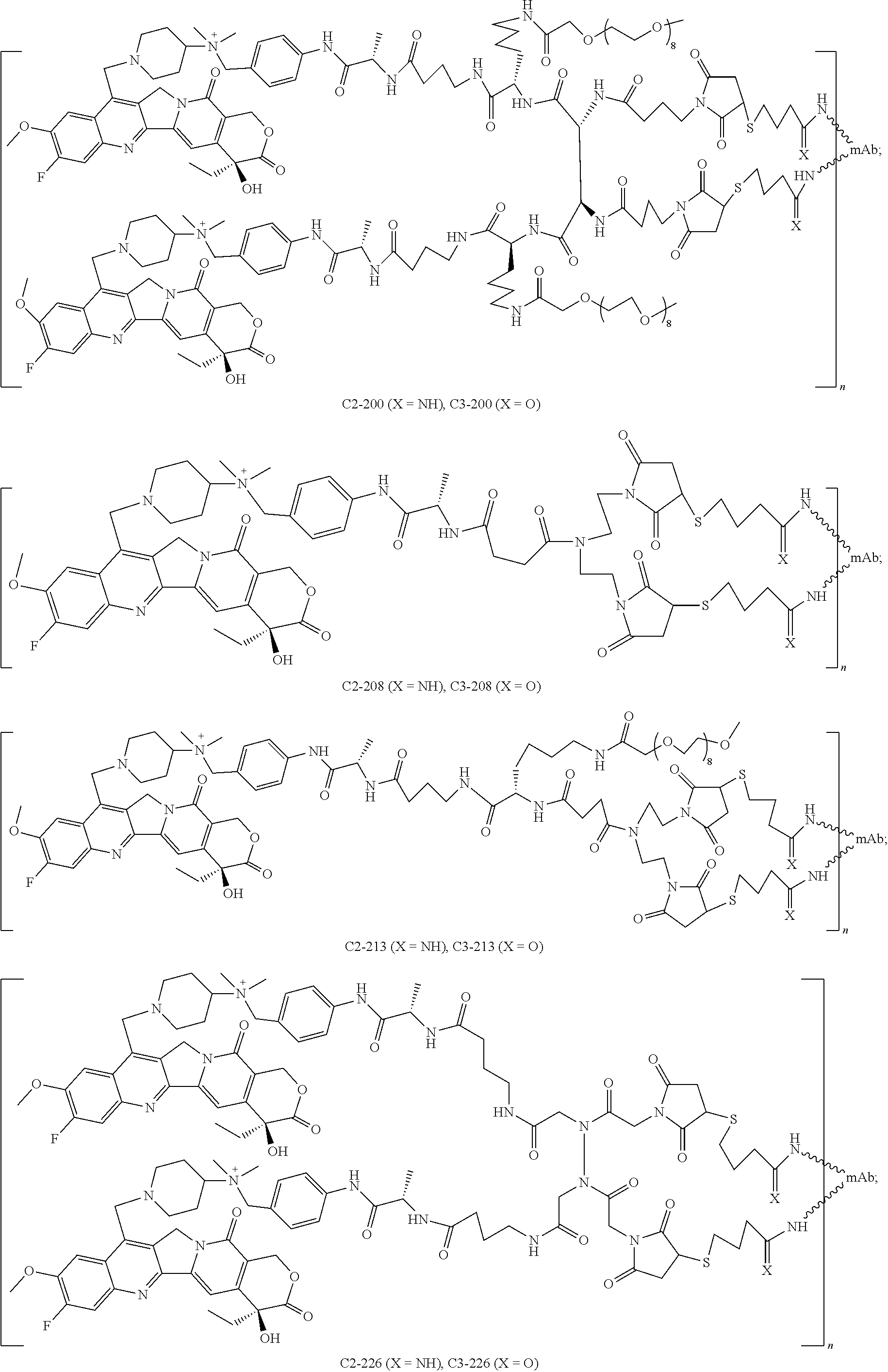 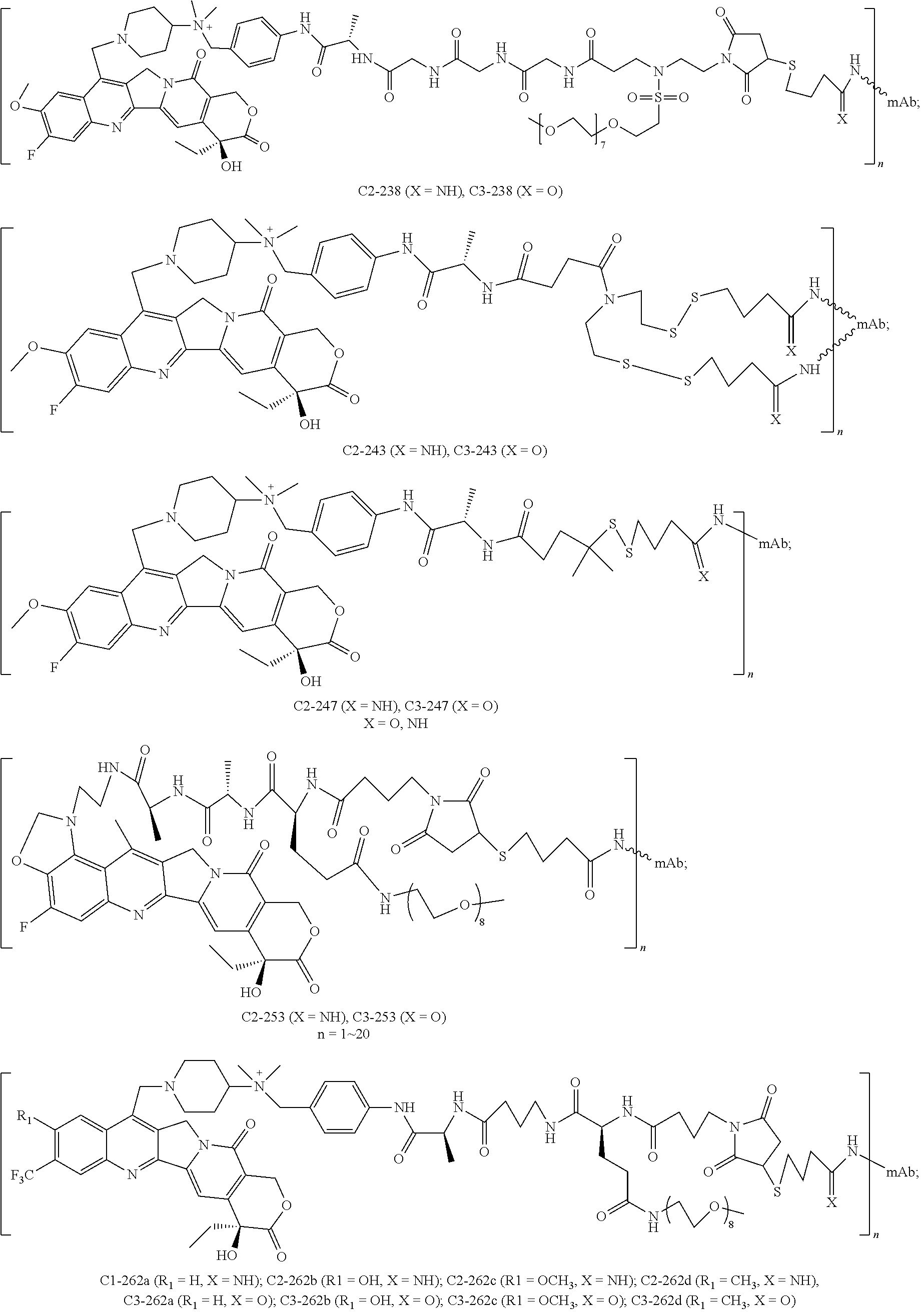 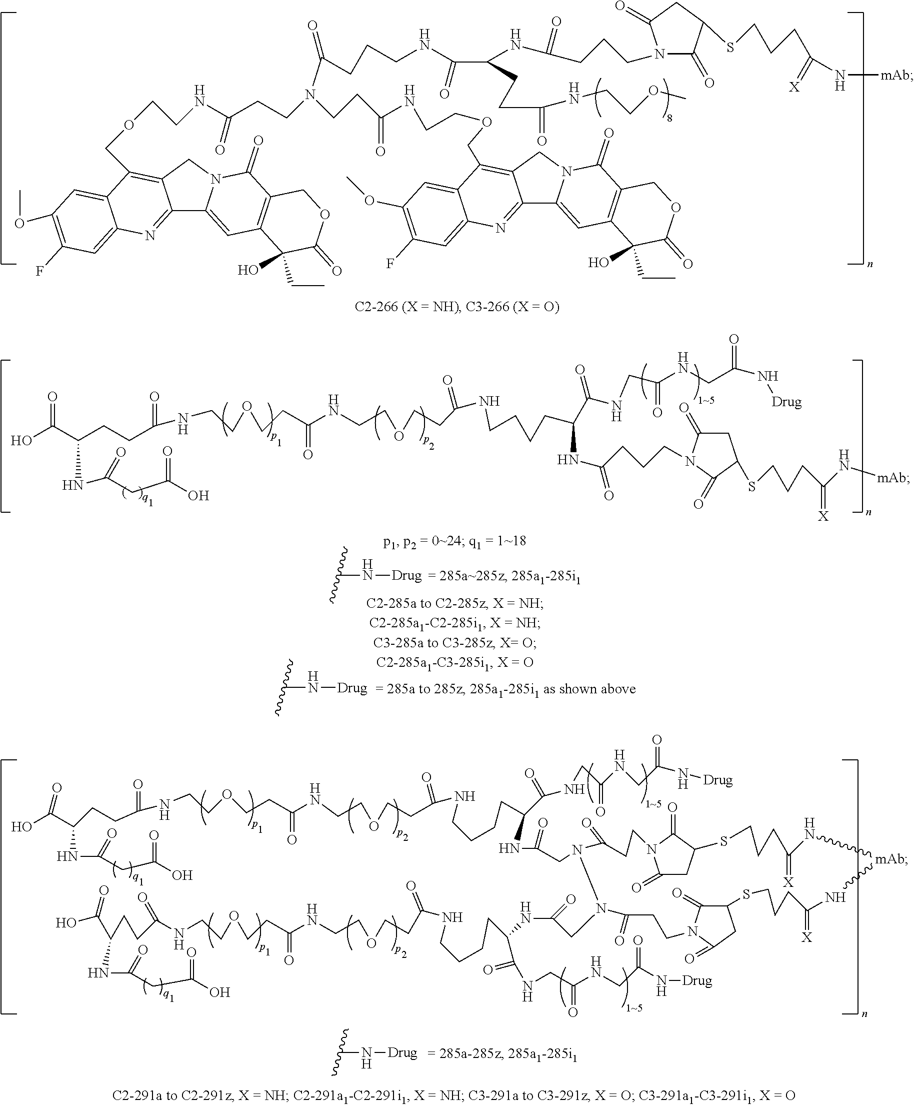 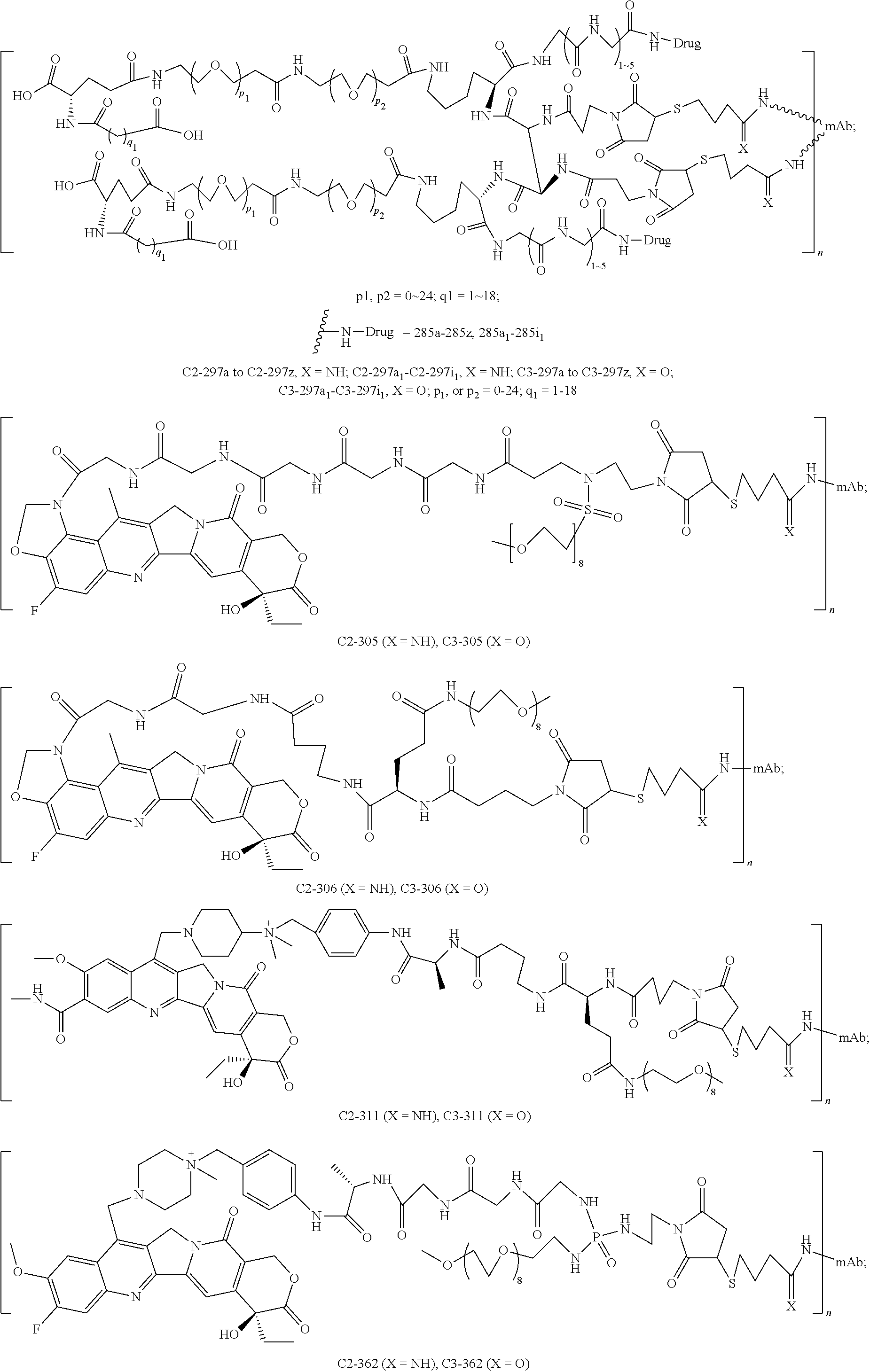 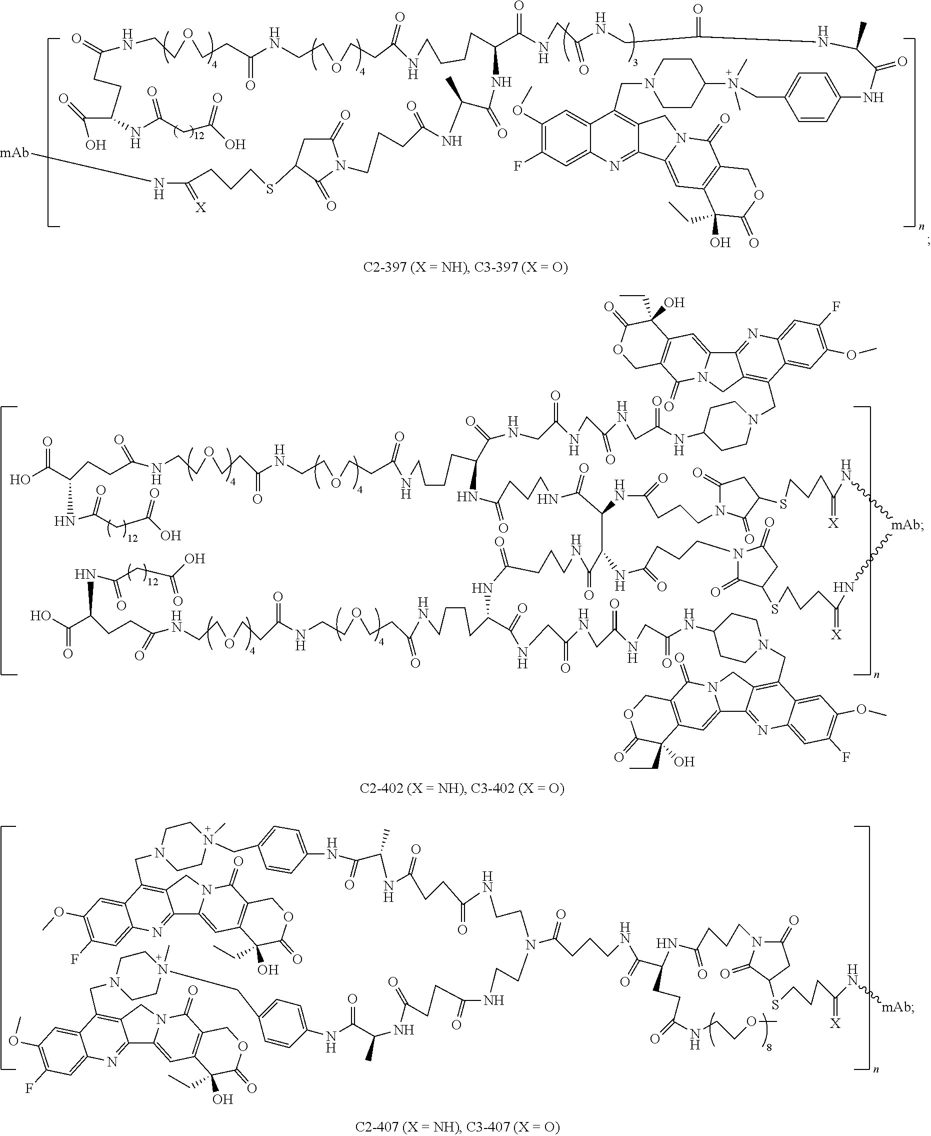 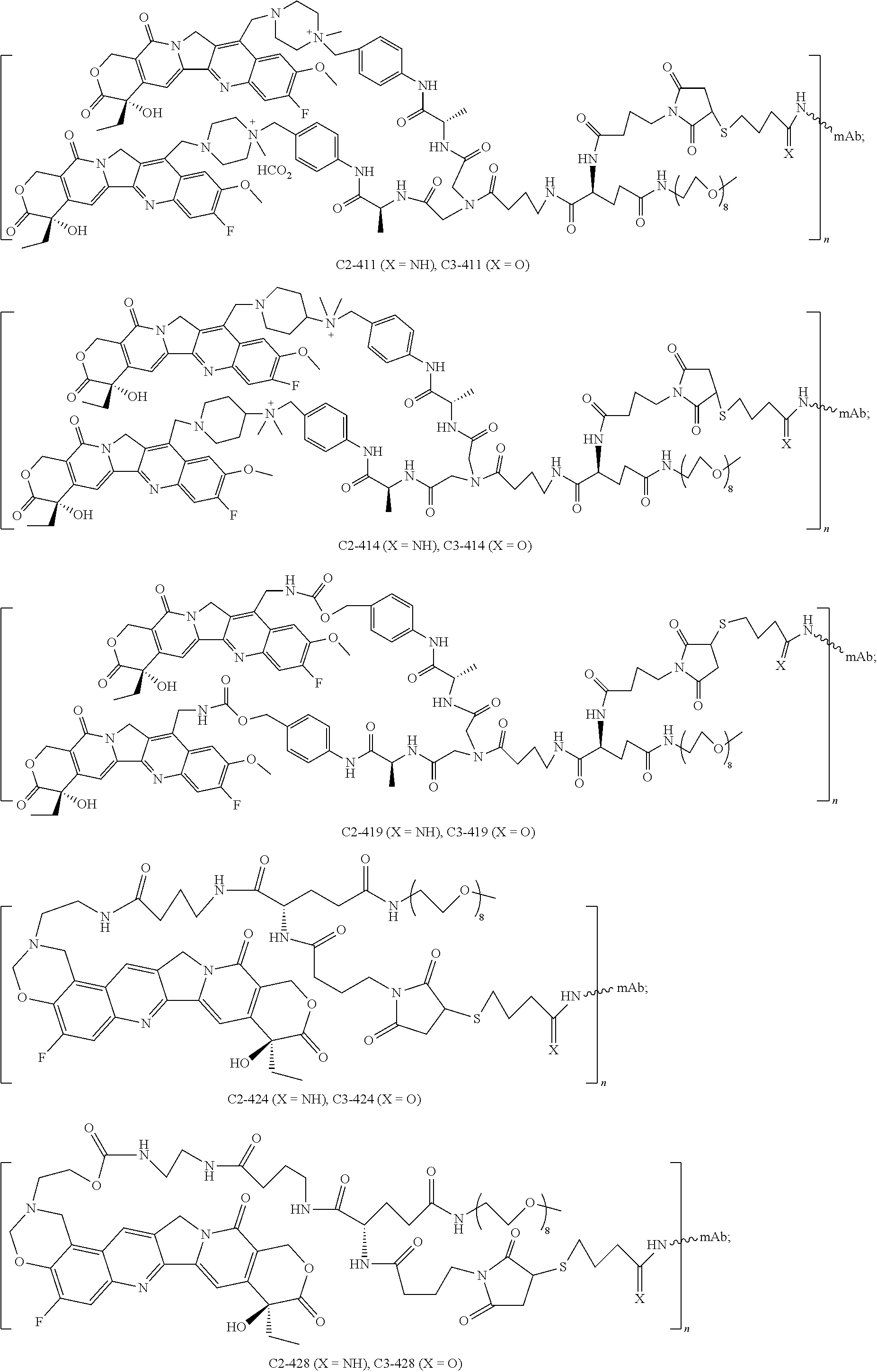
wherein mAb is an antibody.

34. The process according to claim 30, wherein the compound of Formula (VI) has one of structures of compounds 29, 31, 35, 41, 42, 43, 47, 50, 56, 61, 64, 70, 75, 81, 86, 88, 90, 94, 99, 102, 110, 113, 114, 119a, 119b, 123, 127, 131, 137a, 137b, 140, 147a, 147b, 151, 152, 157, 158, 159a, 159b, 165, 166, 168, 170a, 170b, 177, 188, 200, 208, 213, 226, 238, 243, 247, 262a, 262b, 262c, 262d, 266, 285 (285a to 285z, 285a₁ to 285i₁), 291, 297, 305, 306, 311, 362, 397, 402, 436, below:

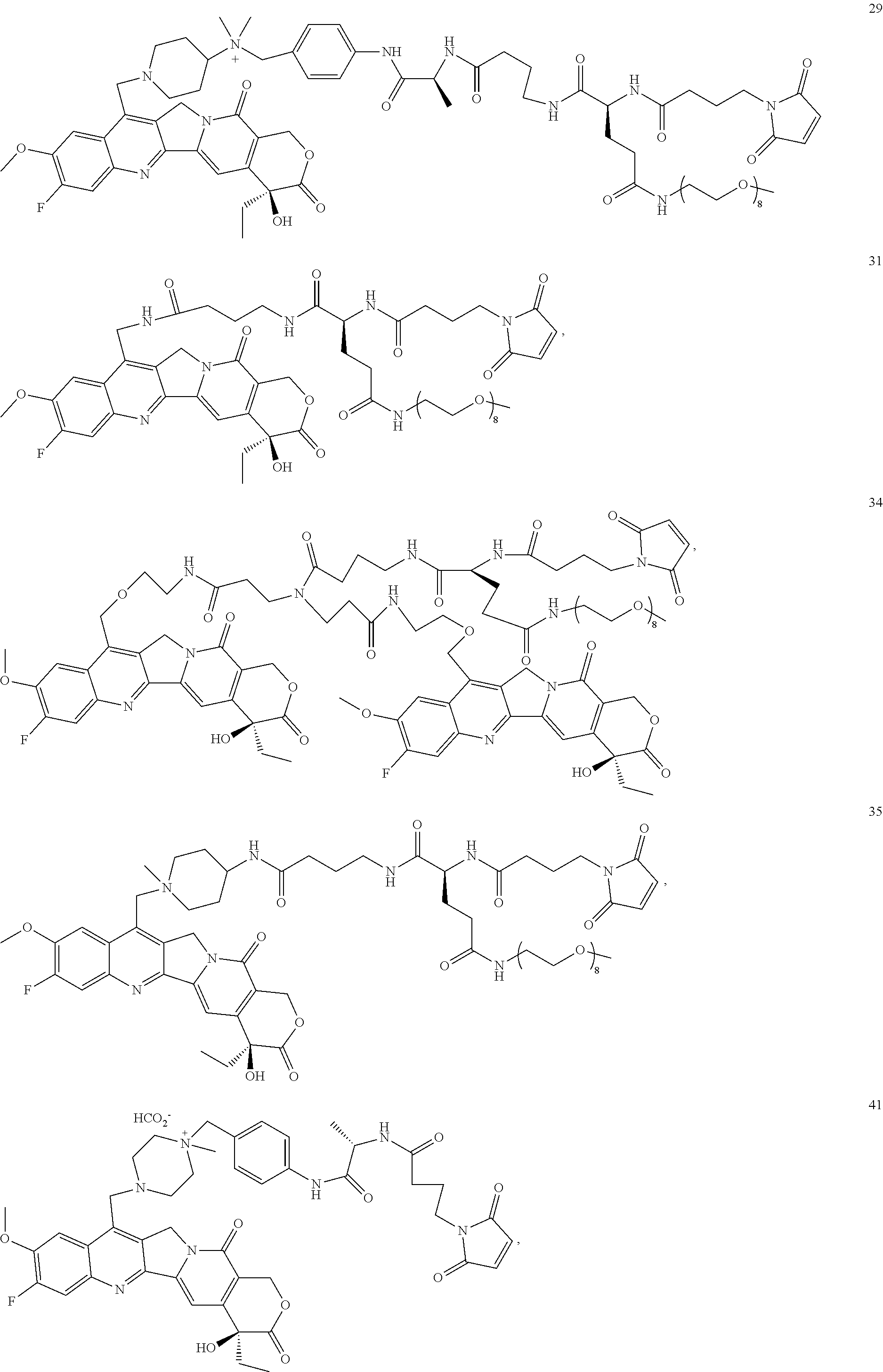

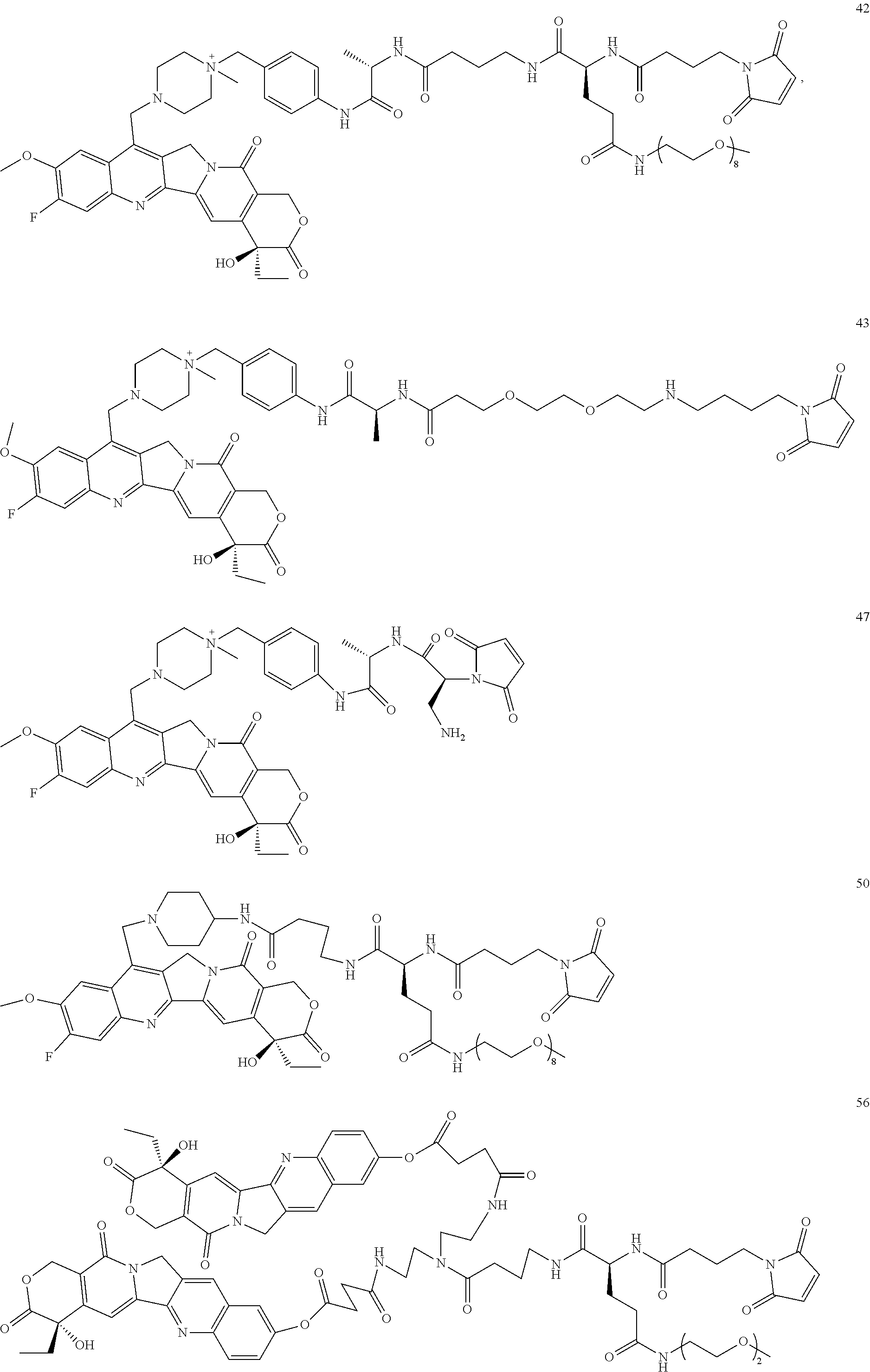

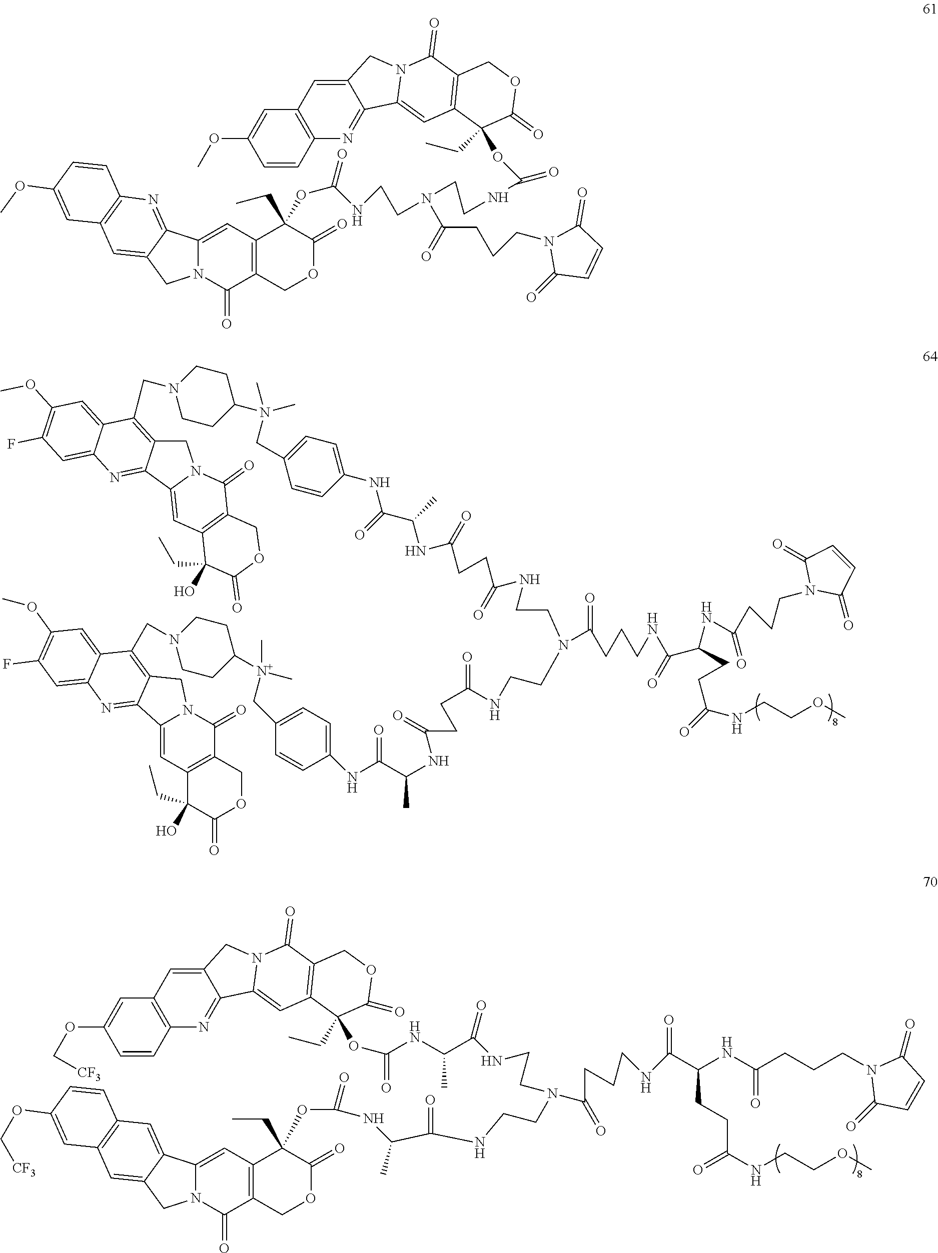

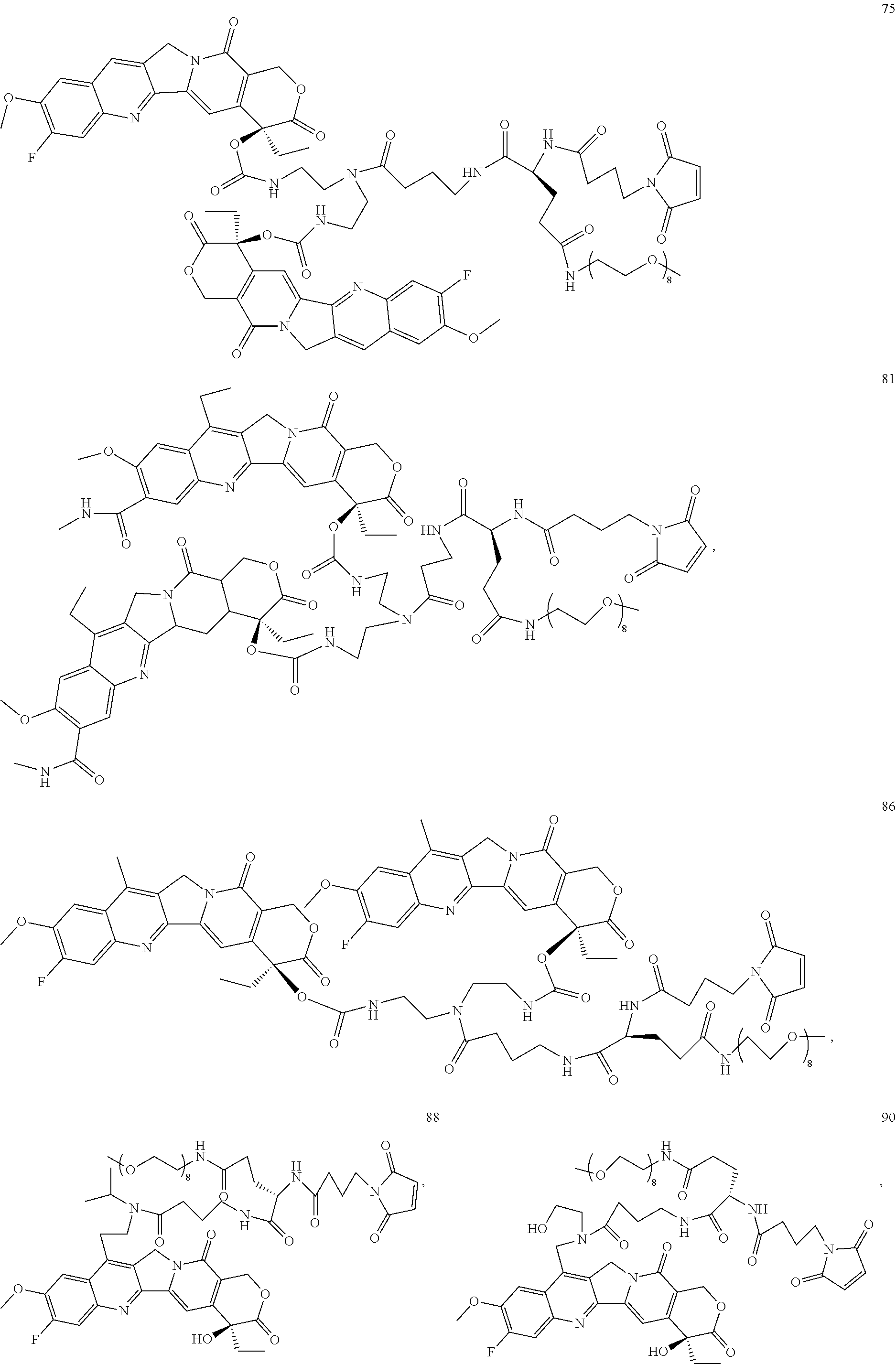

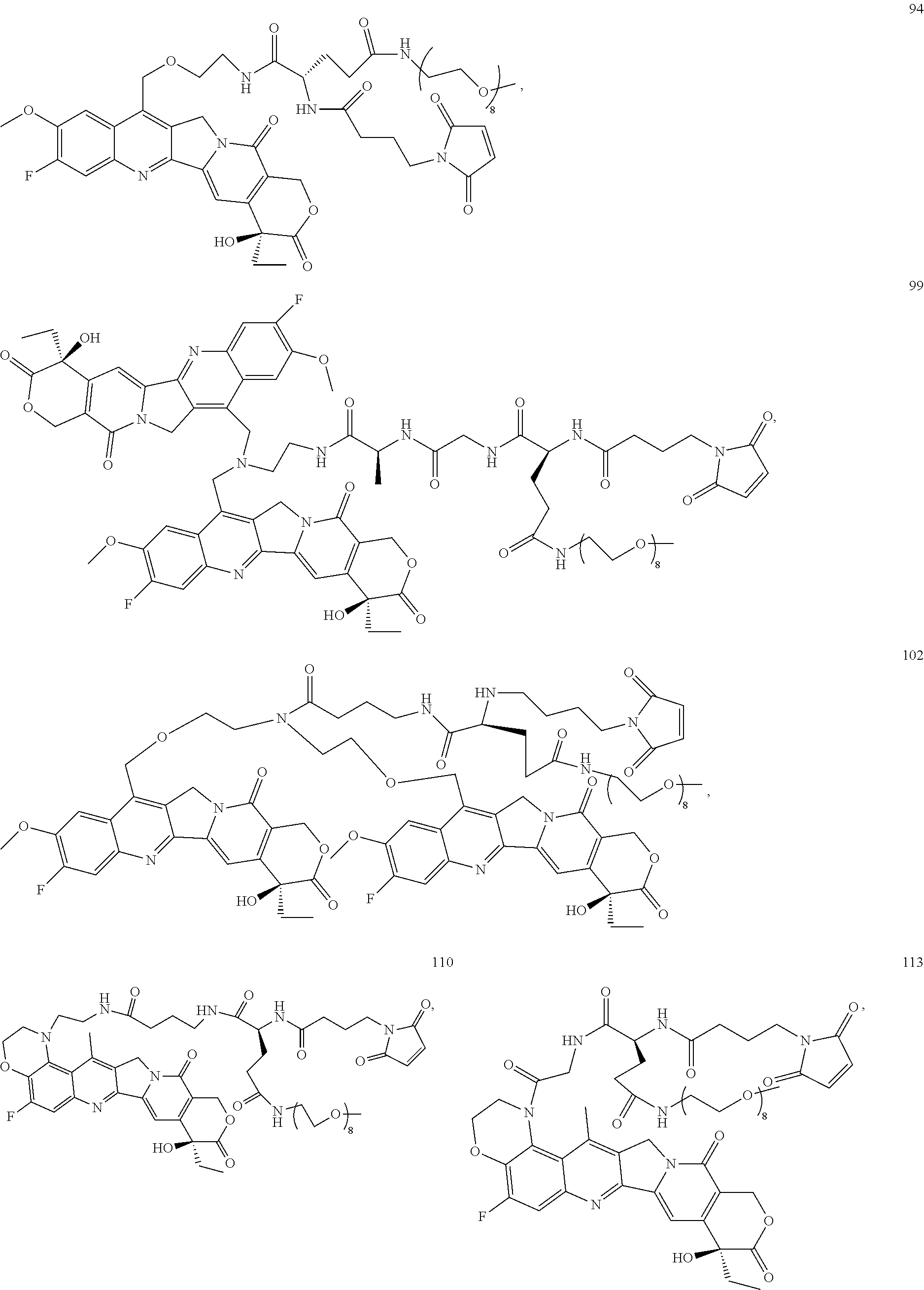

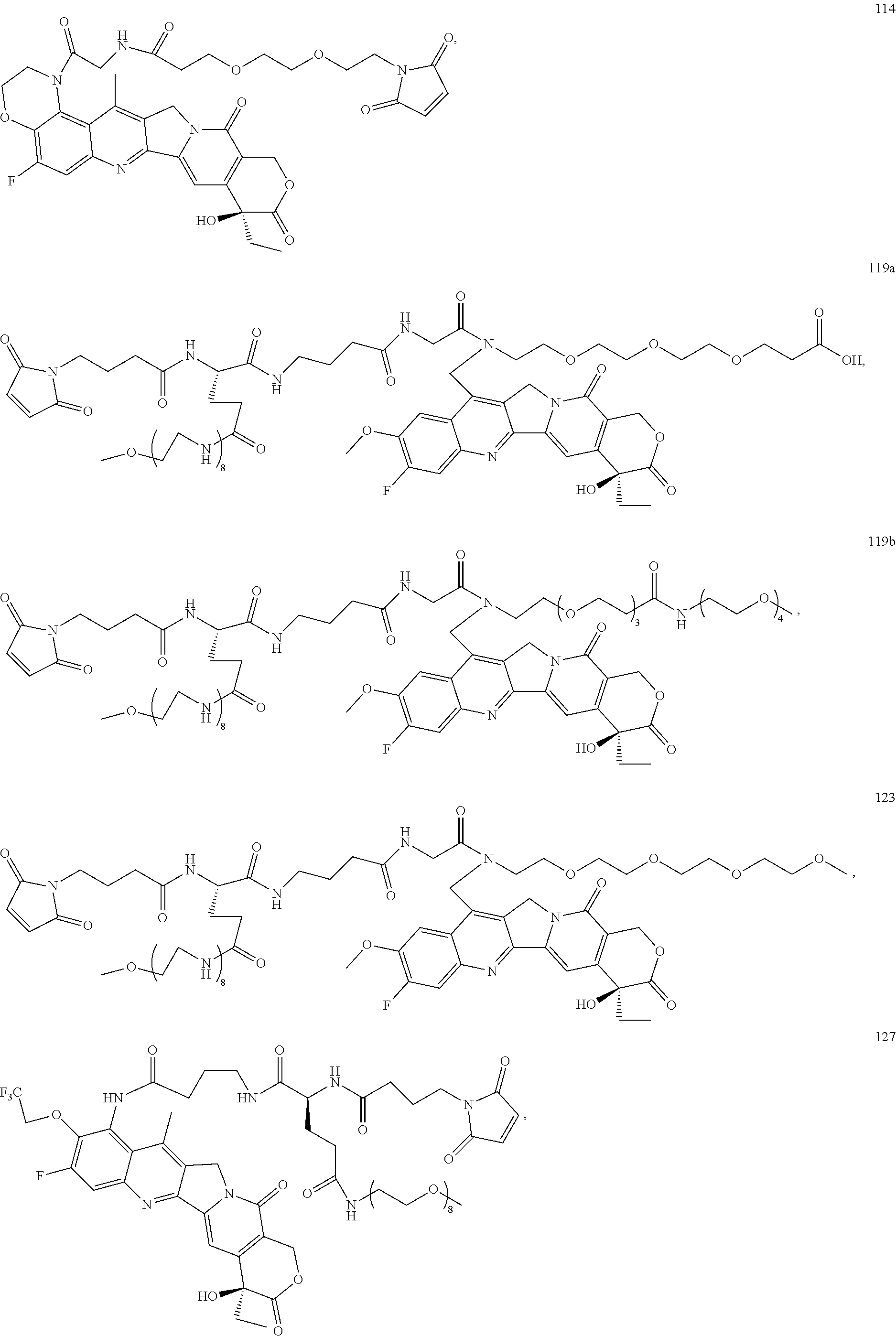

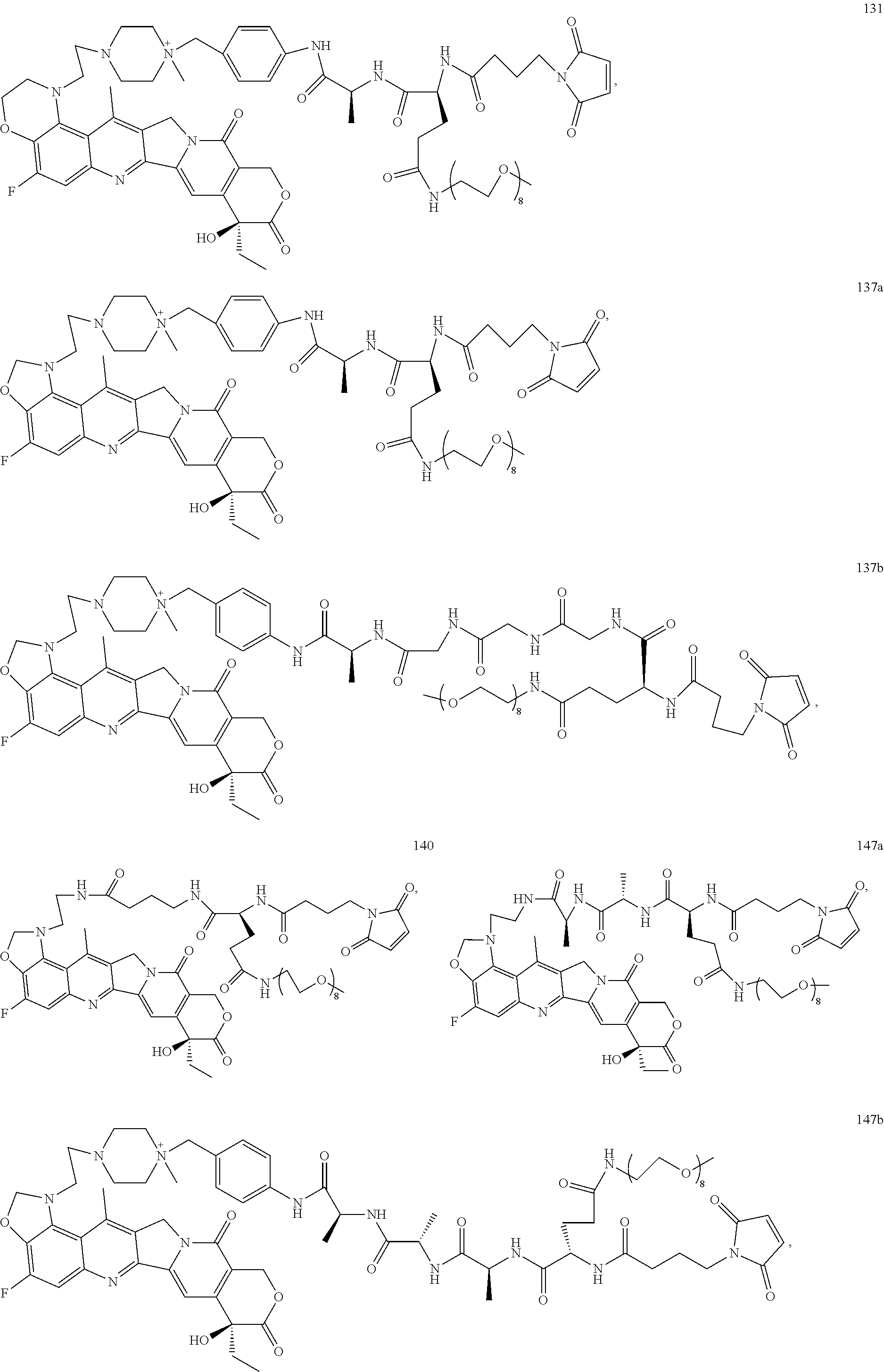

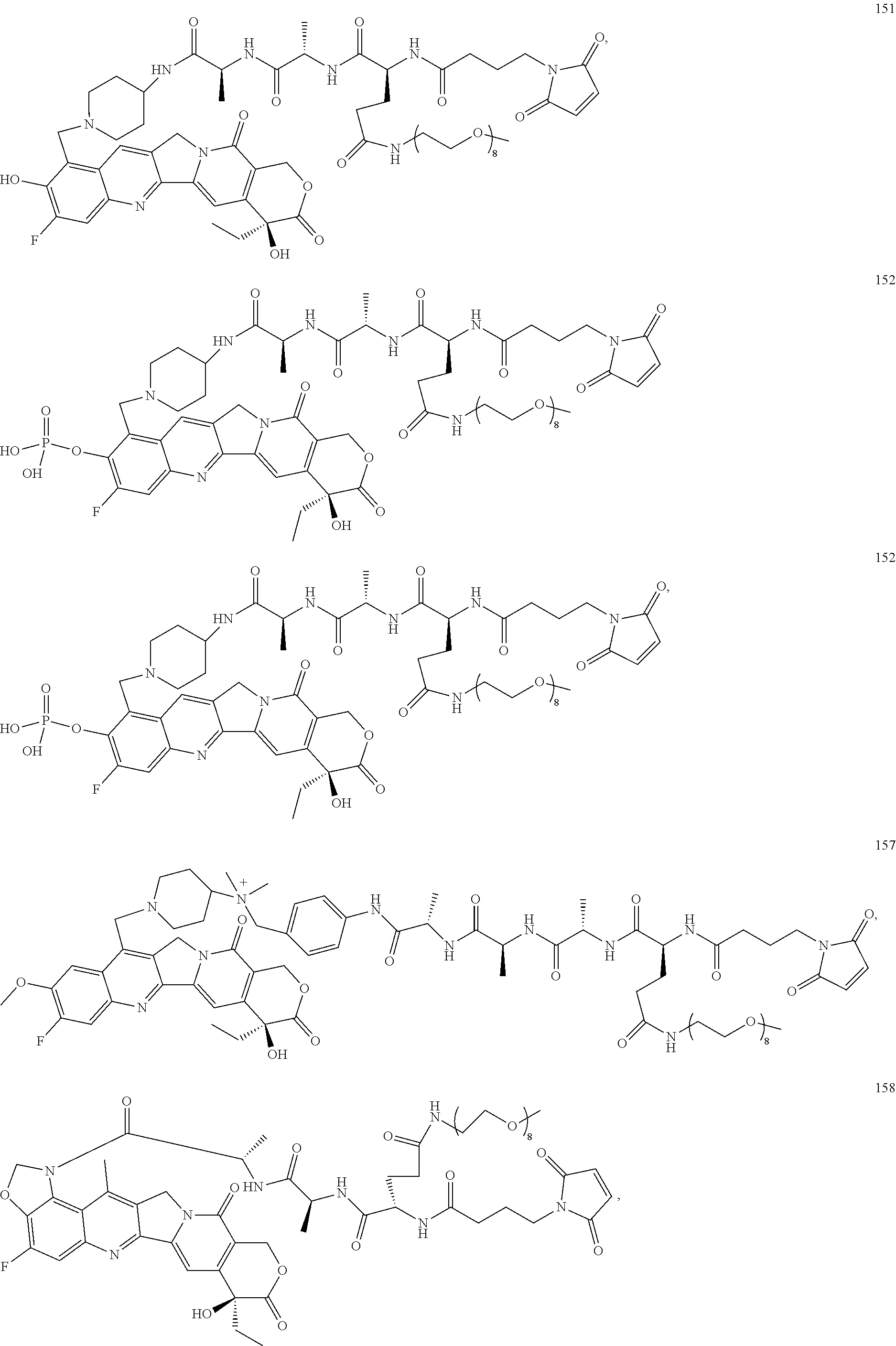

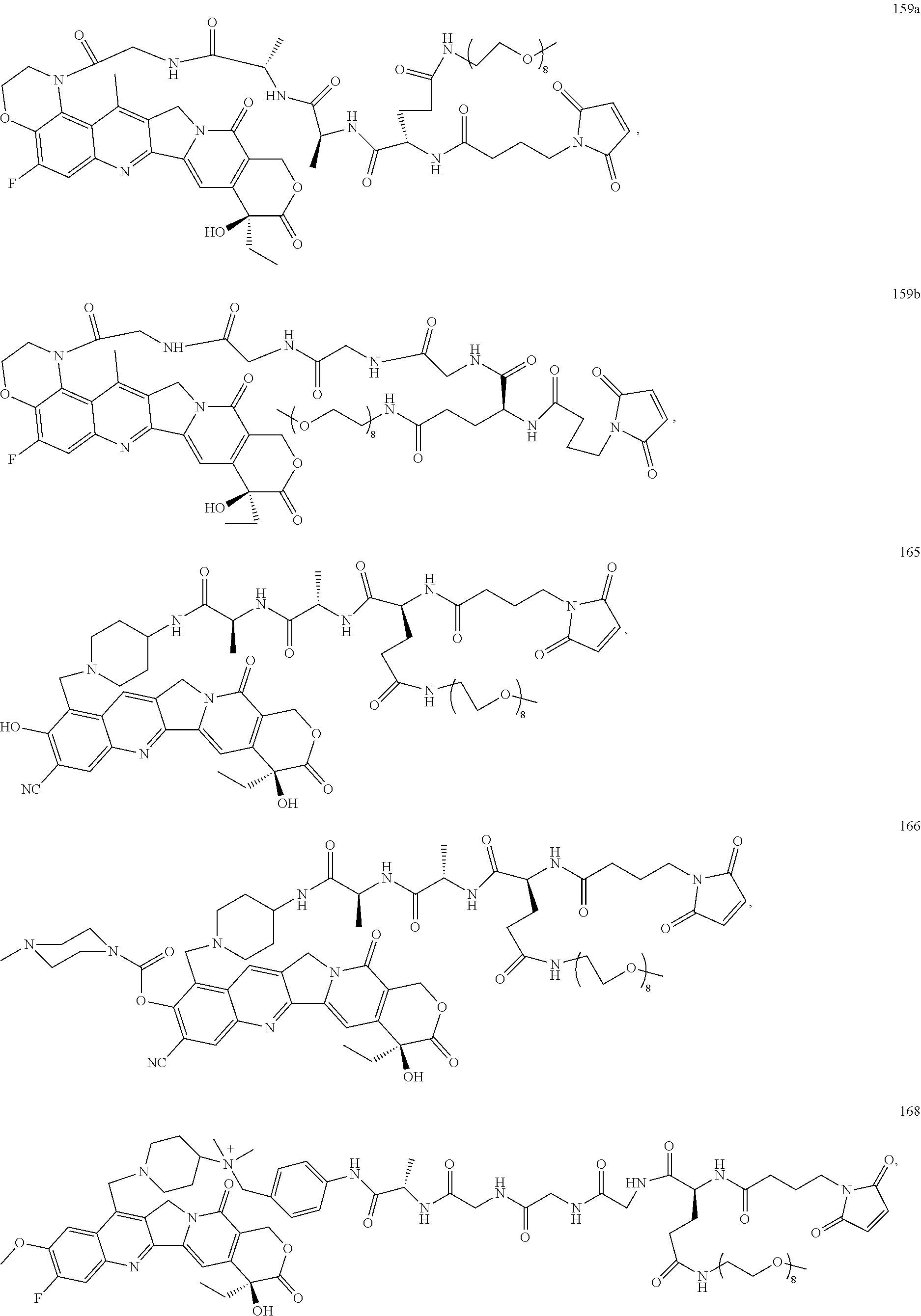

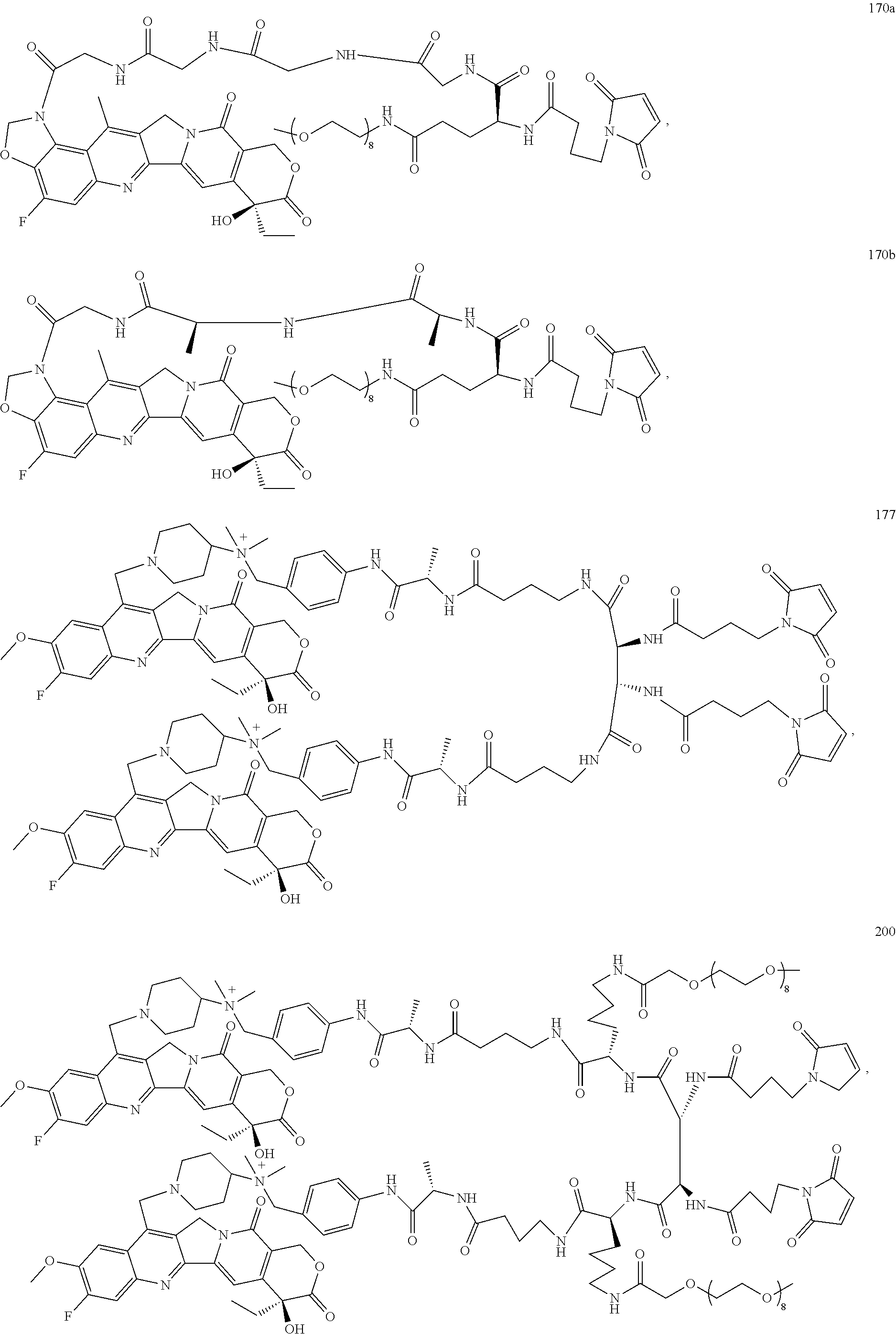

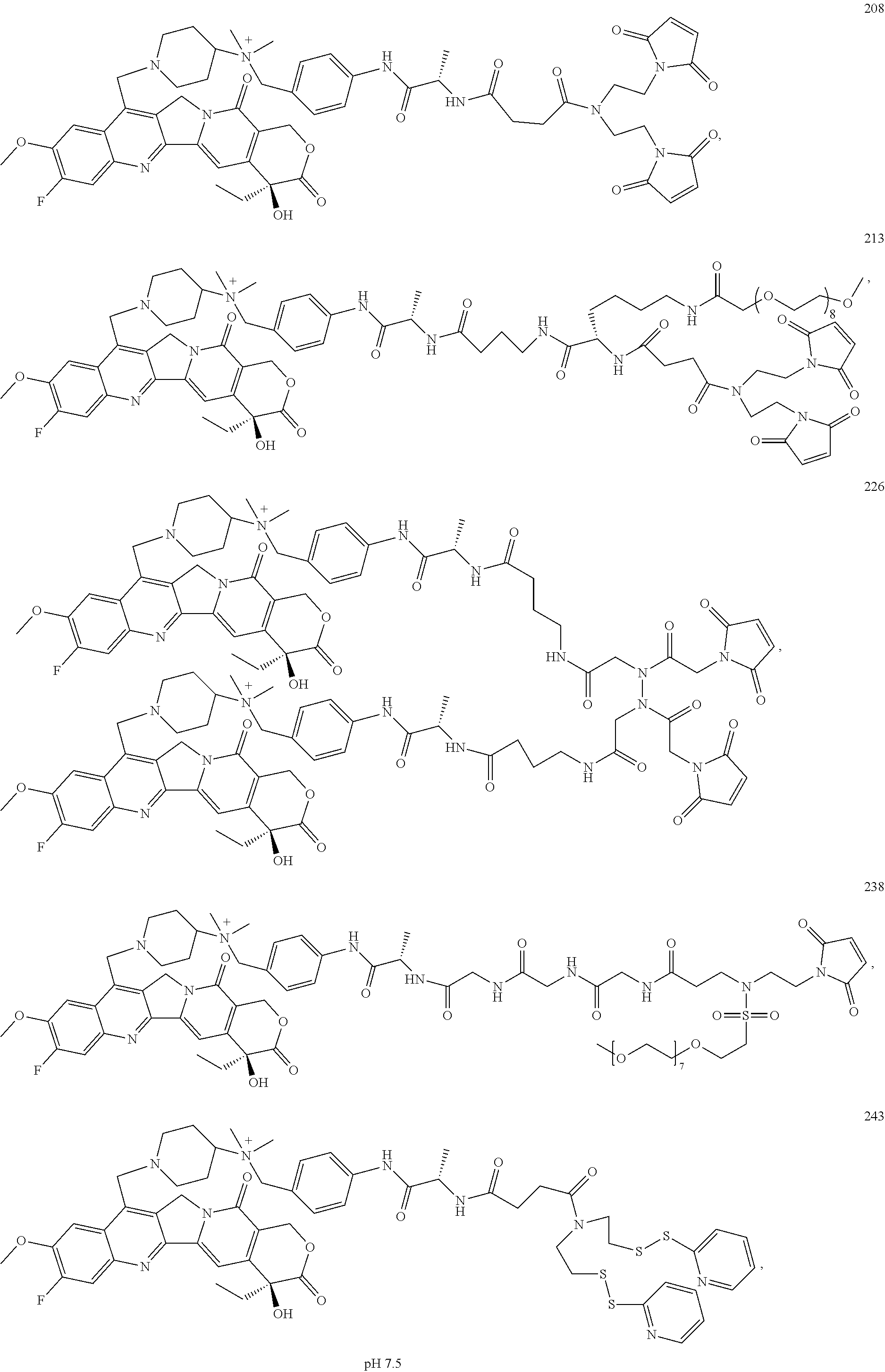

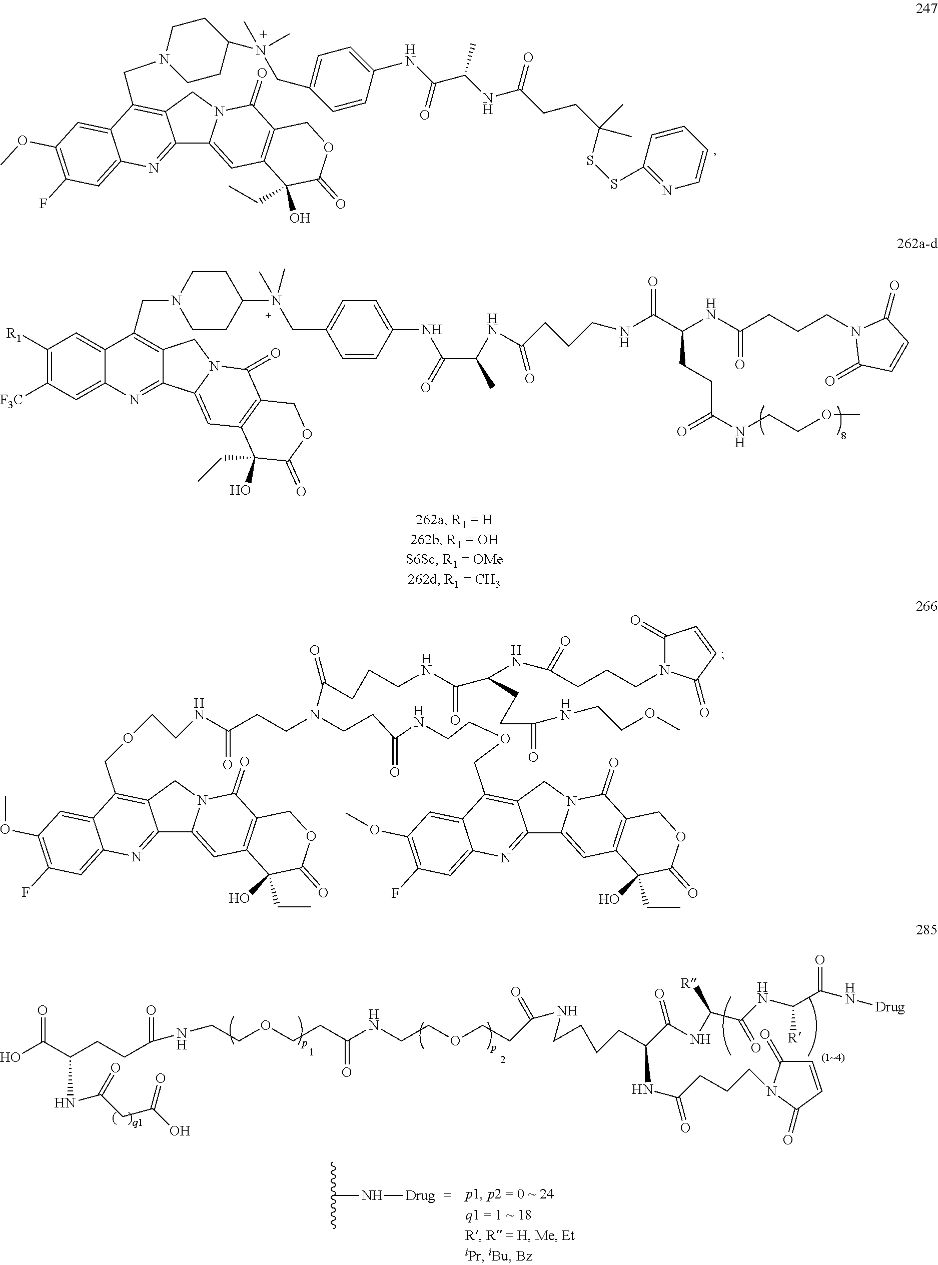

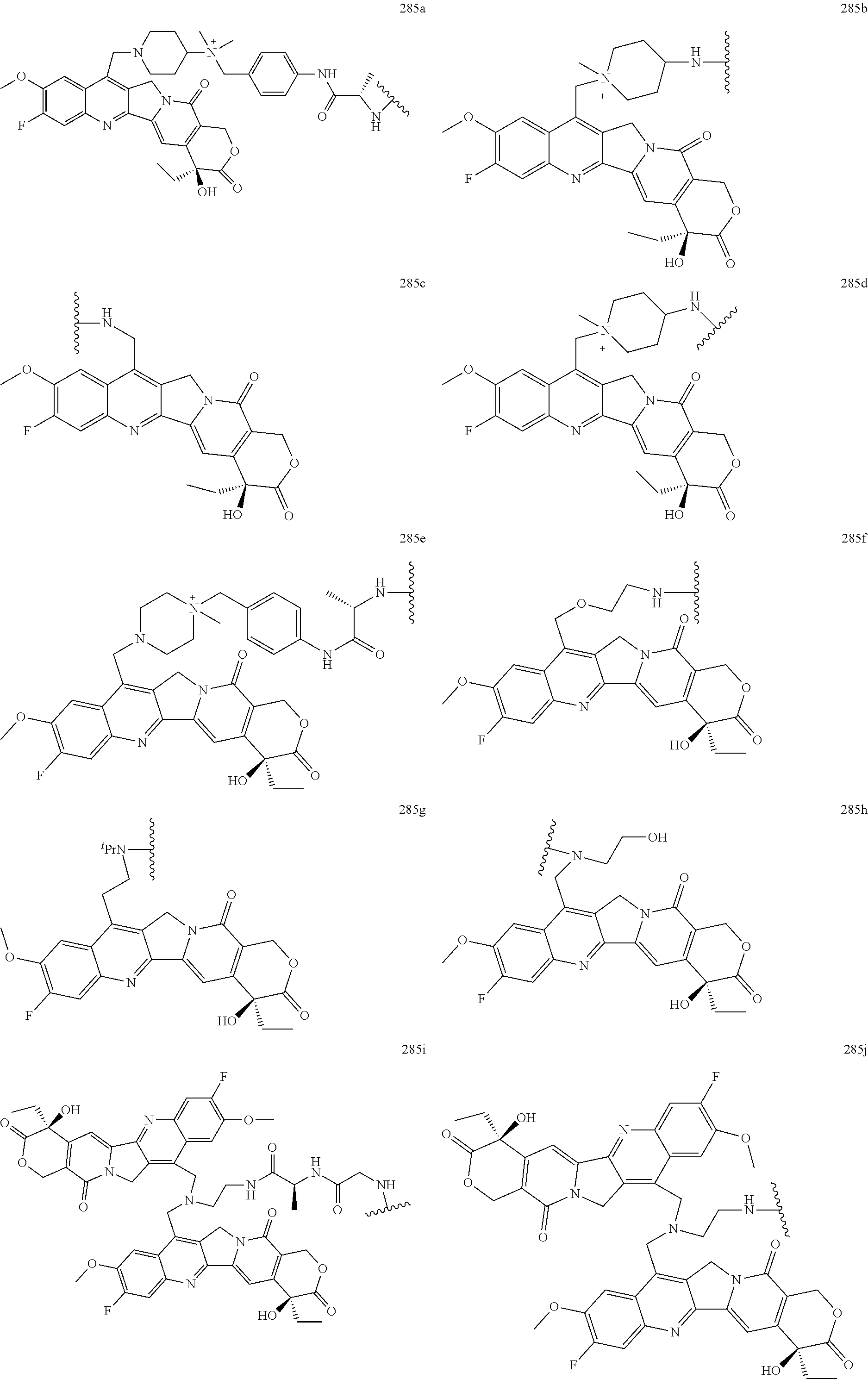

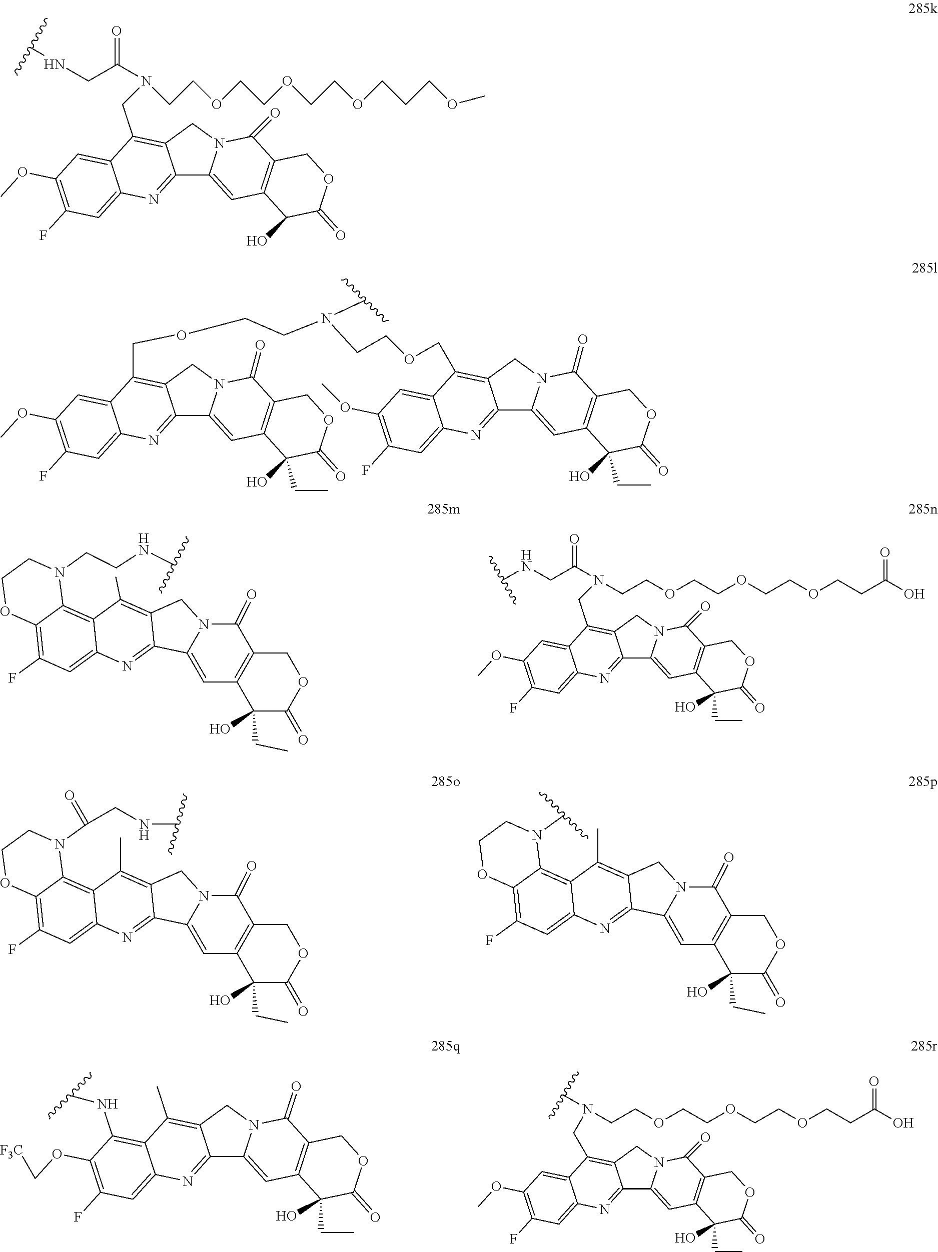

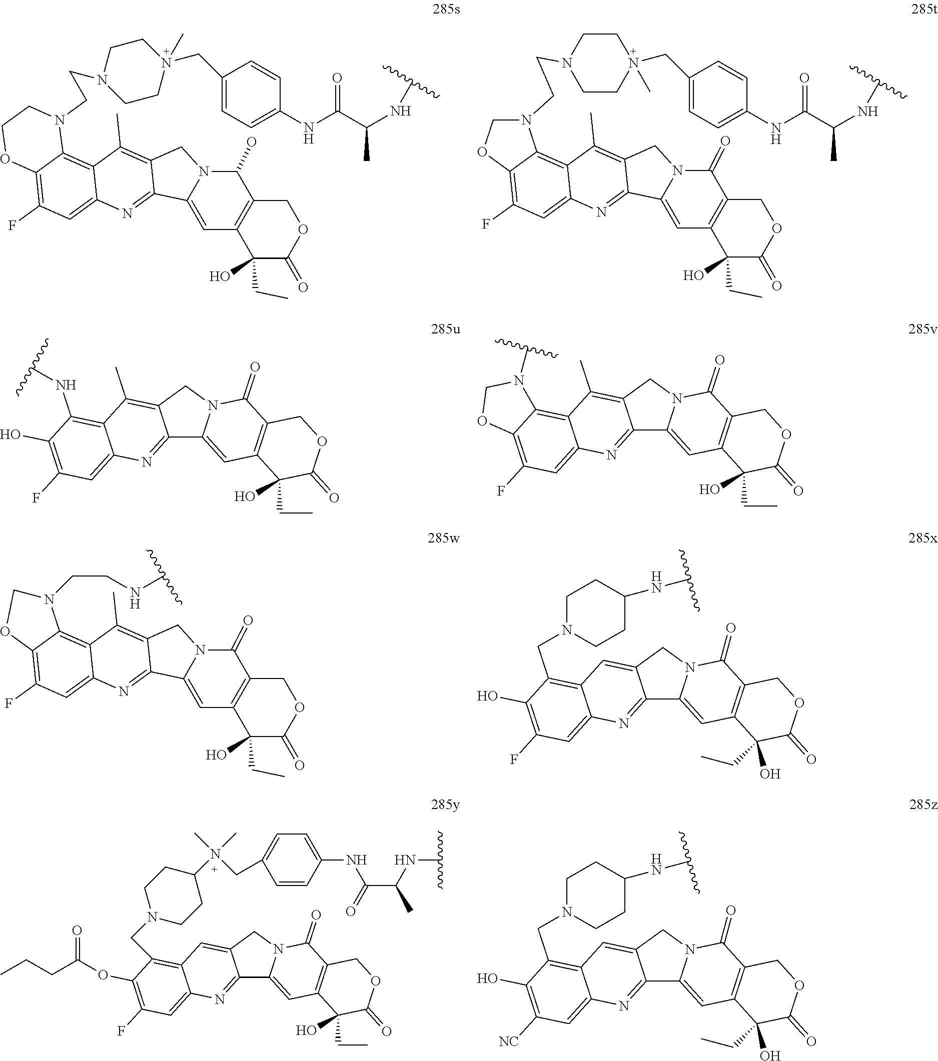

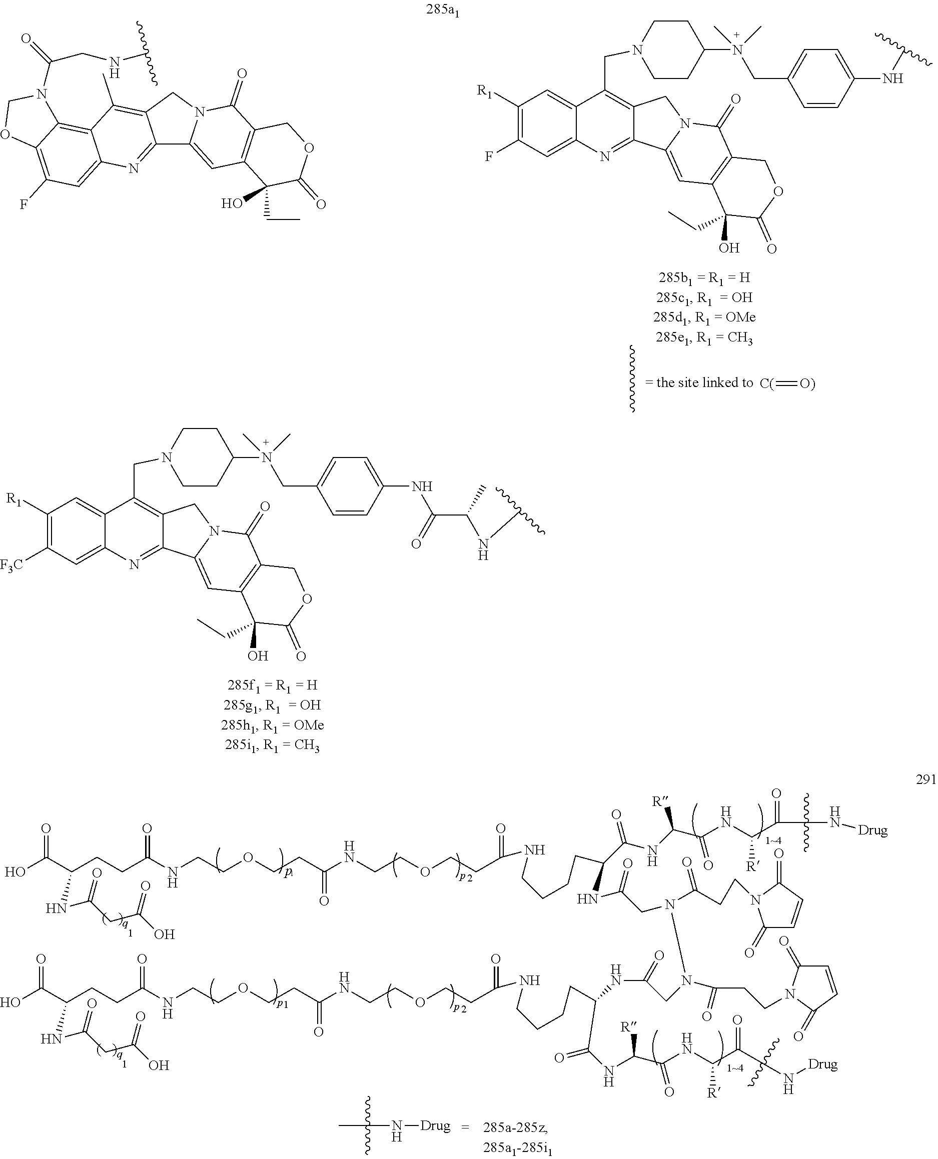

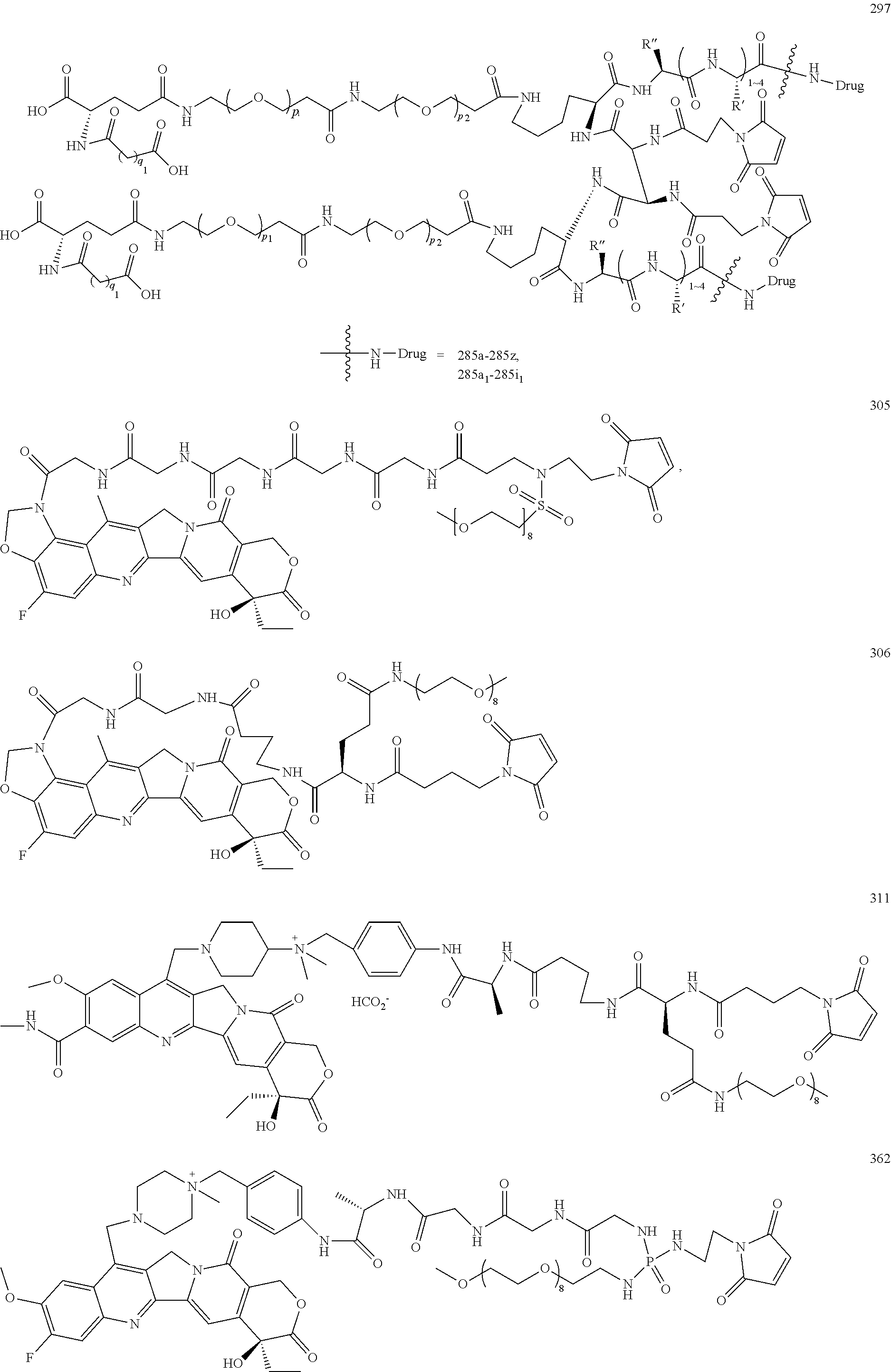

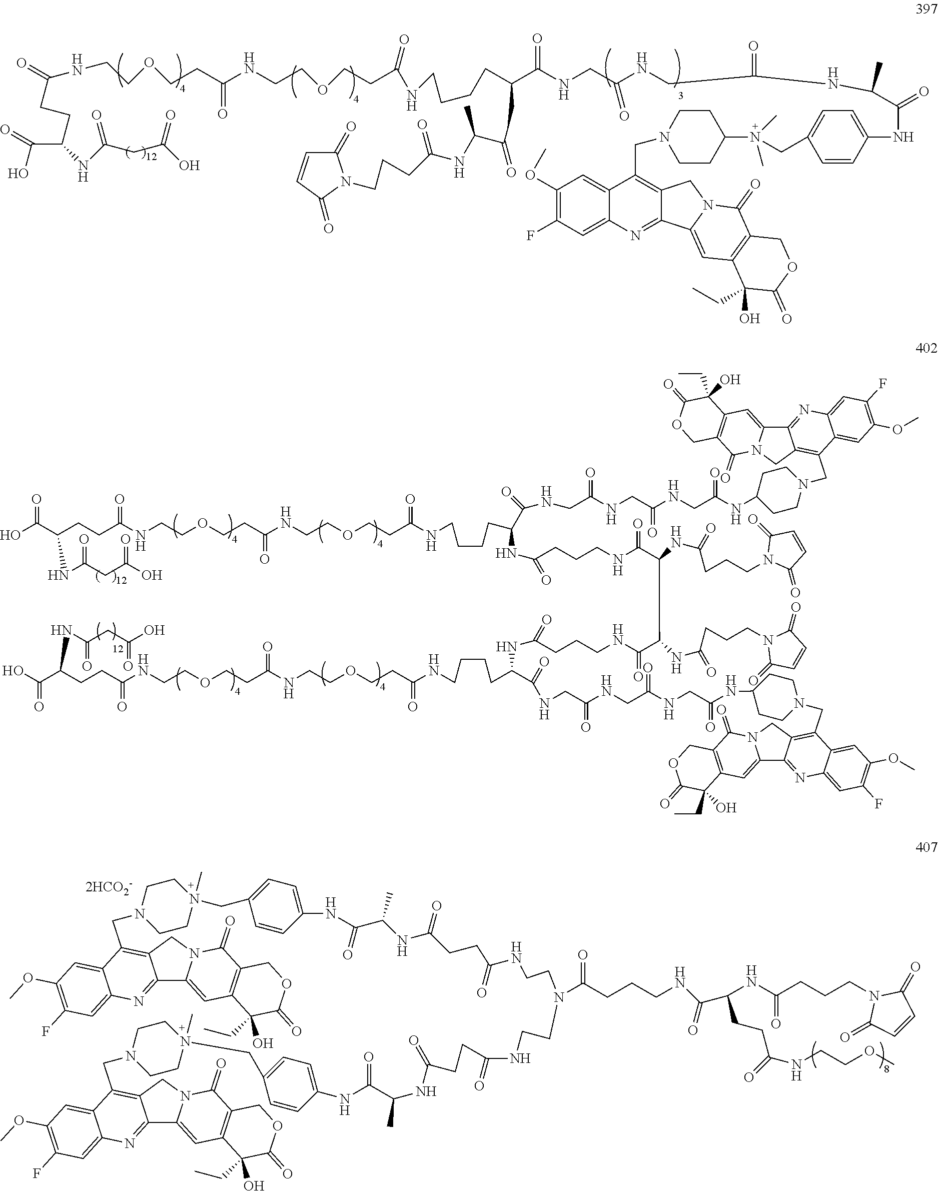

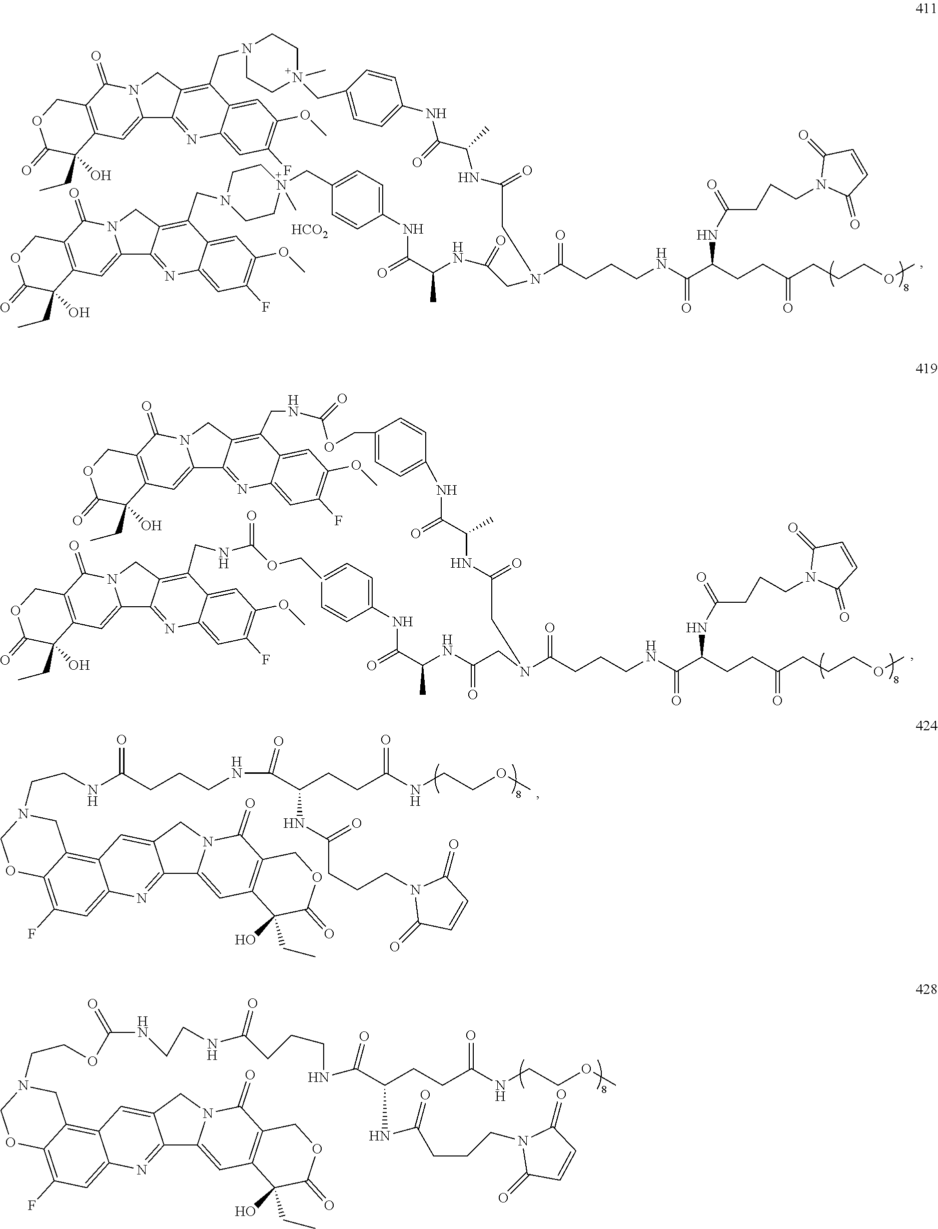

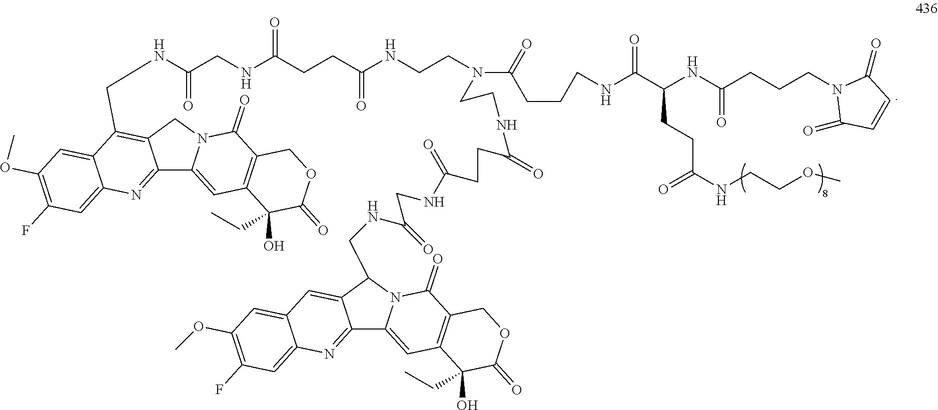

35. The conjugate according to claim 22, wherein the cell-binding molecule (T or mAb), is selected from:
(A): the group consisting of an antibody, a protein, probody, nanobody, a vitamin (including folate), peptides, a polymeric micelle, a liposome, a lipoprotein-based drug carrier, a nano-particle drug carrier, a dendrimer, and a molecule or a particle said above coating or linking with a cell-binding ligand, or a combination of two or more thereof;
(B): an antibody-like protein, a full-length antibody (polyclonal antibody, monoclonal antibody, antibody dimer, antibody multimer), multispecific antibody (selected from, bispecific antibody, trispecific antibody, or tetraspecific antibody); a single chain antibody, an antibody fragment that binds to the target cell, a monoclonal antibody, a single chain monoclonal antibody, a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, diabody, triabody, tetrabody, miniantibody, a probody, a probody fragment, small immune proteins (SIP), a lymphokine, a hormone, a vitamin, a growth factor, a colony stimulating factor, a nutrient-transport molecule, large molecular weight proteins, fusion proteins, kinase inhibitors, gene-targeting agents, nanoparticles or polymers modified with antibodies or large molecular weight proteins;
(C): a cell-binding ligand or receptor agonist selected from: folate derivatives; glutamic acid urea derivatives; Somatostatin and its analogs (selected from the group consisting of octreotide (Sandostatin) and lanreotide (Somatuline)); aromatic sulfonamides; pituitary adenylate cyclase activating peptides (PACAP) (PAC1); vasoactive intestinal peptides (VIP/PACAP) (VPAC1, VPAC2); melanocyte-stimulating hormones (α-MSH); cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); neurotensin receptor ligands (NTR1, NTR2, NTR3); substance P (NK1 receptor) ligands; neuropeptide Y (Y1-Y6); homing peptides include RGD (Arg-Gly-Asp), NGR (Asn-Gly-Arg), the dimeric and multimeric cyclic RGD peptides (selected from cRGDfV), TAASGVRSMH and LTLRWVGLMS (chondroitin sulfate proteoglycan NG2 receptor ligands) and F3 peptides; cell penetrating peptides (CPPs); peptide hormones, selected from the group consisting of luteinizing hormone-releasing hormone (LHRH) agonists and antagonists, and gonadotropin-releasing hormone (GnRH) agonist, acting by targeting follicle stimulating hormone (FSH) and luteinizing hormone (LH), as well as testosterone production, selected from the group consisting of Buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), Leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); pattern recognition receptor (PRRs), selected from the group consisting of Toll-like receptors' (TLRs) ligands, C-type lectins and nodlike receptors' (NLRs) ligands; Calcitonin receptor agonists; integrin receptors' and their receptor subtypes' (selected from the group consisting of αᵥβ₁, αᵥβ₃, αᵥβ₅, αᵥβ₆, α₆β₄, α₇β₁, α_{L}β₂, α_{IIb}β₃) agonists (selected from the group consisting of GRGDSPK, cyclo(RGDfV) (L1) and its derives [cyclo(-N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; nanobody (a derivative of VHH (camelid Ig)); domain antibodies (dAb, a derivative of VH or VL domain); bispecific T cell engager (BiTE, a bispecific diabody); dual affinity ReTargeting (DART, a bispecific diabody); tetravalent tandem antibodies (TandAb, a dimerized bispecific diabody); anticalin (a derivative of lipocalins); adnectins (10th FN3 (Fibronectin)); designed ankyrin repeat proteins (DARPins); avimers; EGF receptors, or VEGF receptors' agonists;
(D): a small molecule of cell-binding molecule/ligand or a cell receptor agonist selected from the following: LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αᵥβ₃ integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor), LB23 (for an androgen receptor), LB24 (Cilengitide/cyclo(-RGDfV-) for an αᵥ integrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a nucleoside analog), LB53 (Erlotinib analog) or LB54 (Lapatinib analog) as shown in the following structures:

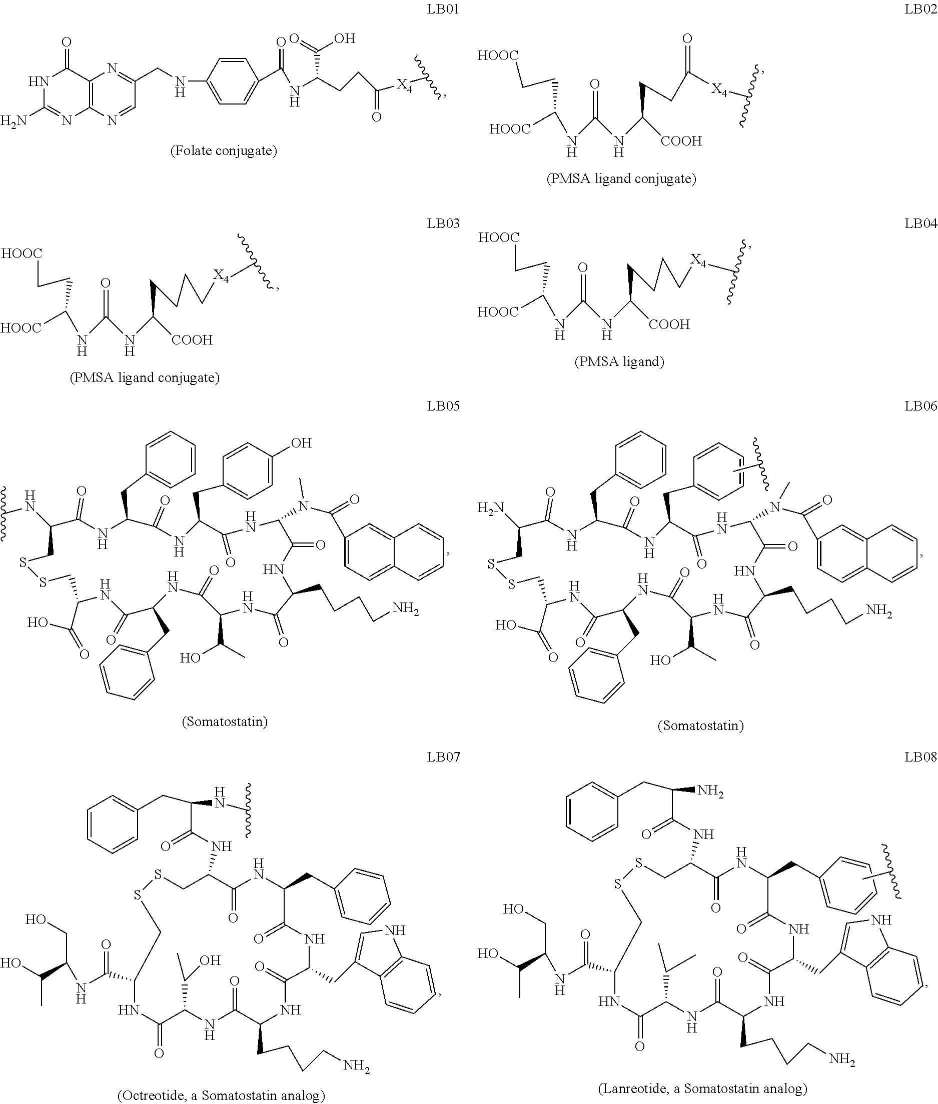

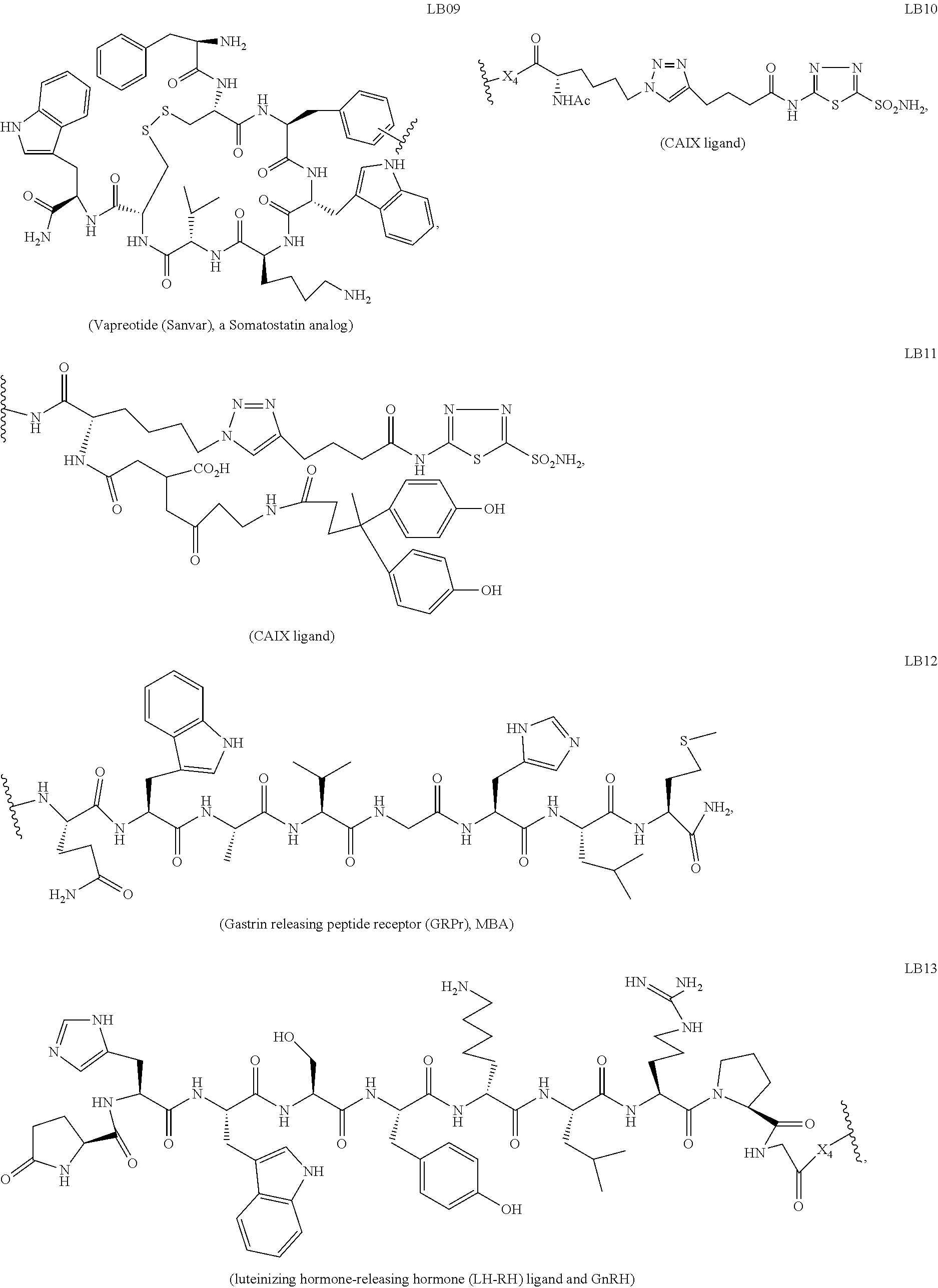

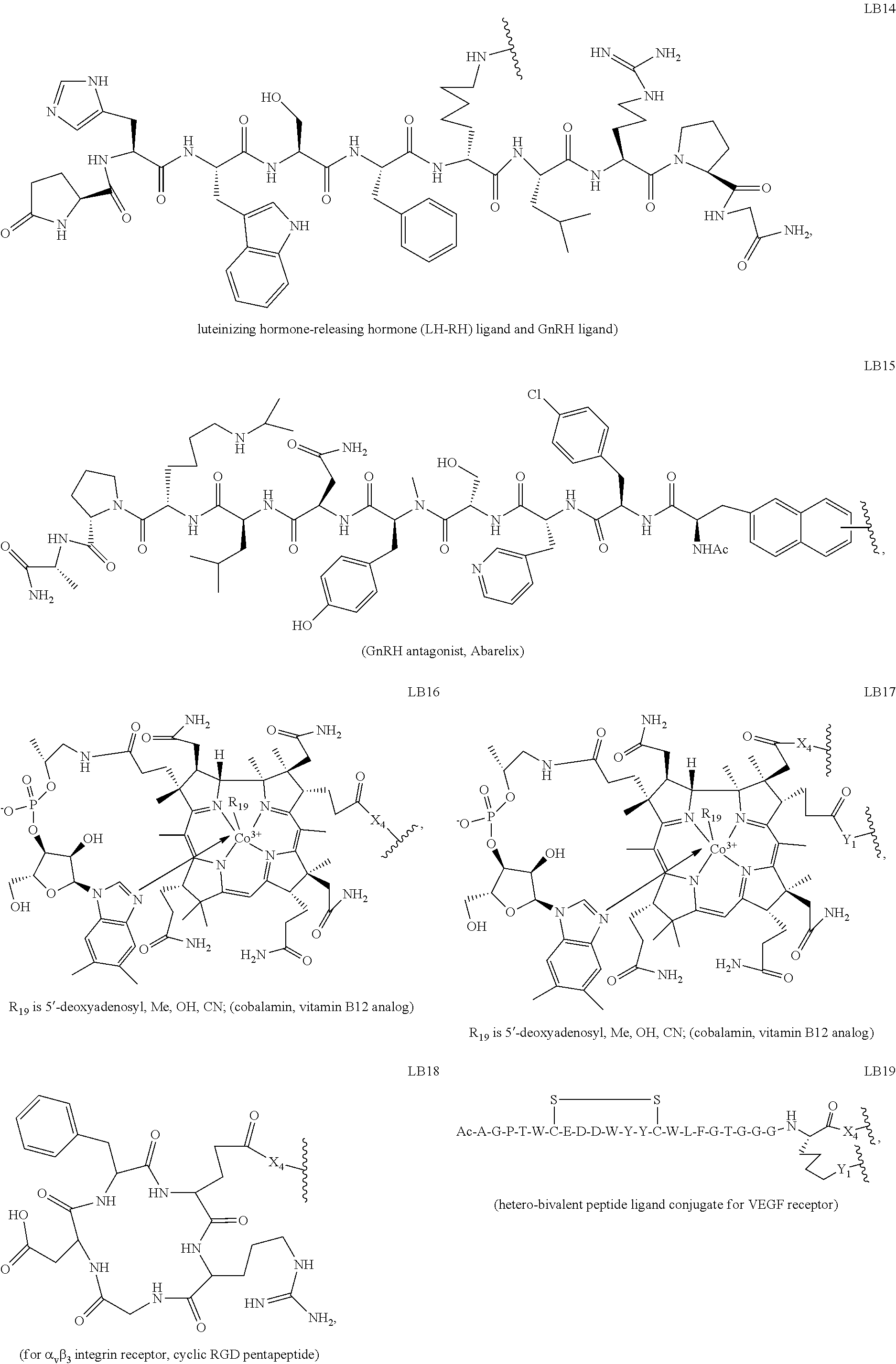

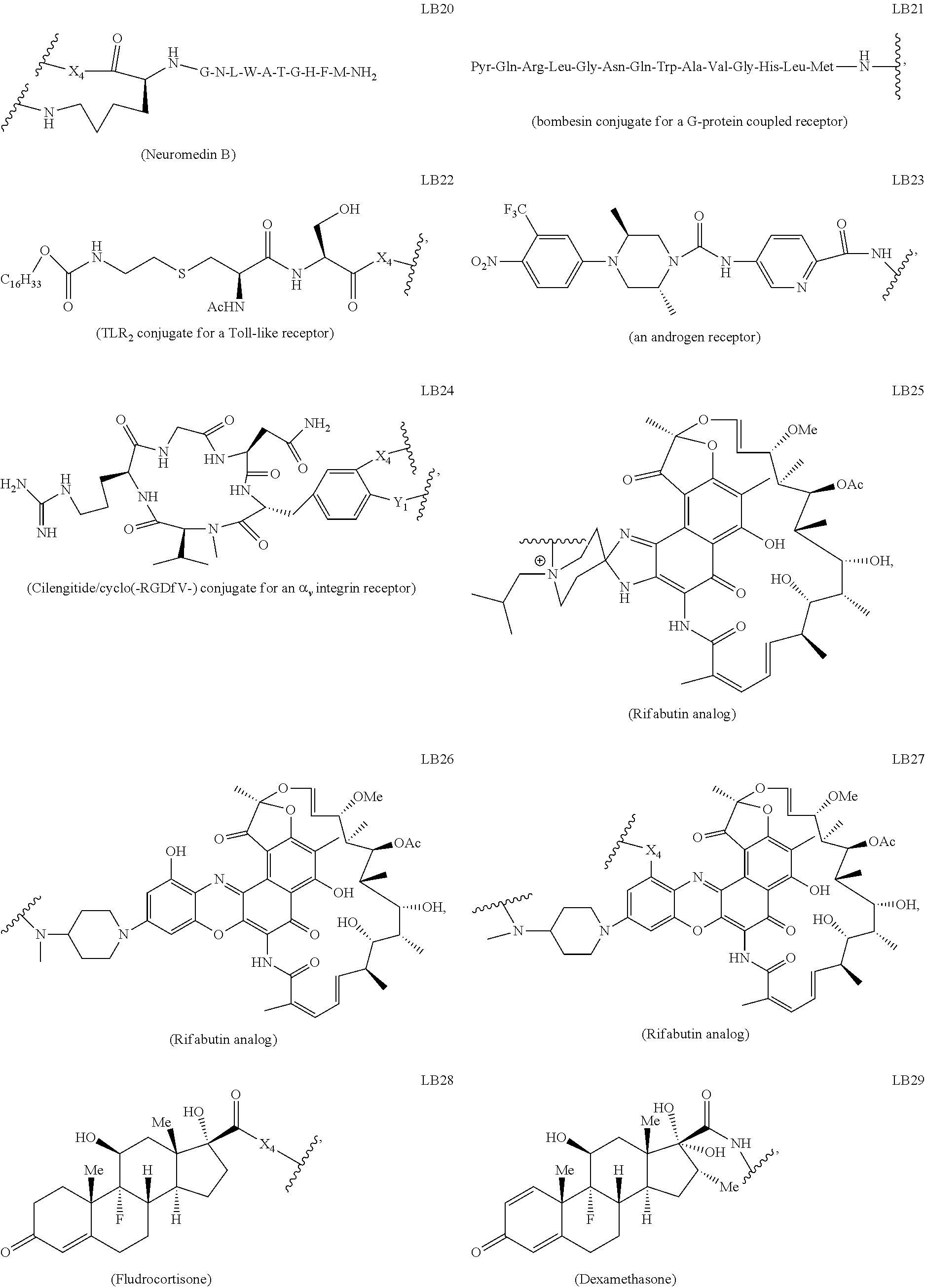

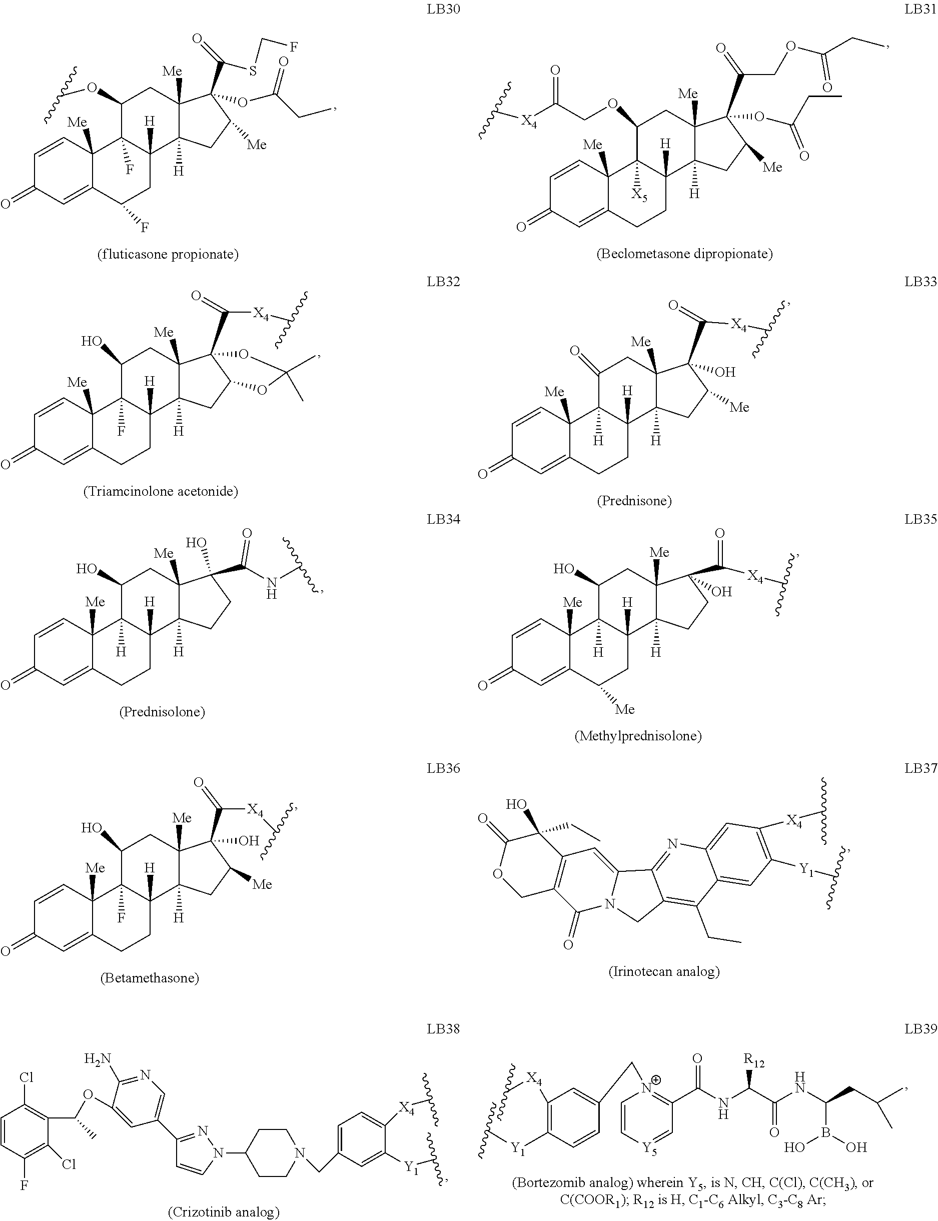

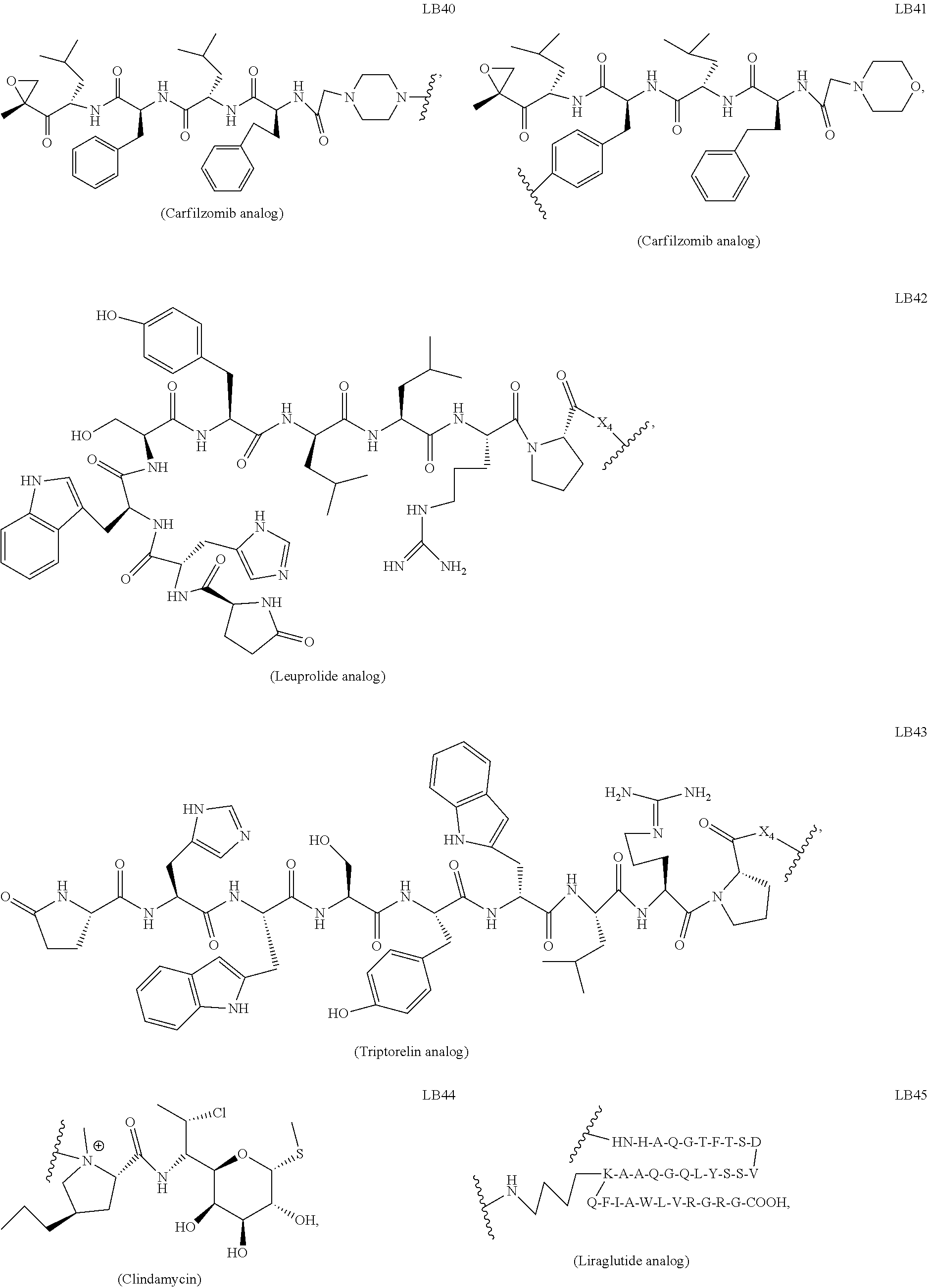

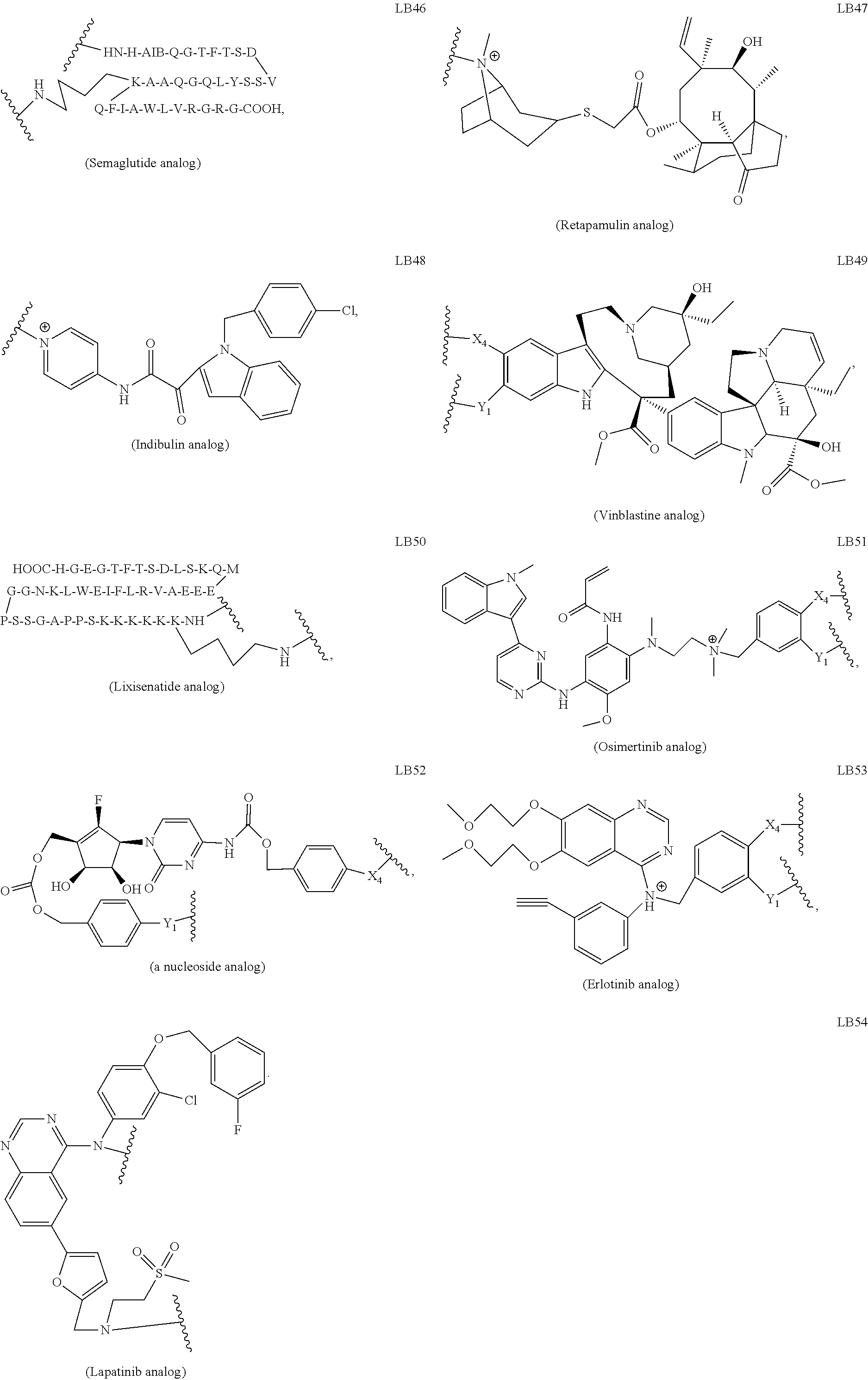

36. The conjugate according to claim 22, wherein the cell-binding molecule is capable of targeting against a tumor cell, a virus infected cell, a microorganism infected cell, a parasite infected cell, an autoimmune disease cell, an activated tumor cell, a myeloid cell, an activated T-cell, an affecting B cell, or a melanocyte, or a cell expressing any one of the following antigens or receptors: CD1, CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD12w, CD13, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202(a,b), CD203, CD203c, CD204, CD205, CD206, CD207, CD208, CD209, CD210, CDw210a, CDw210b, CD211, CD212, CD213, CD213a, CD213a₂, CD214, CD215, CD216, CD217, CD218, CD218a, CD218, CD21b9, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR1, CRGF, CRIPTO, CXCR5, LY64, TDGF1, 4-1BB, APO2, ASLG659, BMPR1B, 4-1BB, 5AC, 5T4 (trophoblastic glycoprotein, TPBG, 5T4, Wnt-activated inhibitory factor 1 or WAIF1), adenocarcinoma antigen, AGS-5, AGS-22M6, activin receptor-like kinase 1, AFP, AKAP-4, ALK, alpha integrin, alpha v beta6, amino-peptidase N, Amyloid beta, androgen receptor, angiopoietin 2, angiopoietin 3, annexin A1, anthrax toxin protective antigen, anti-transferrin receptor, AOC3 (VAP-1), B7-H3, Bacillus anthracis anthrax, BAFF (B-cell activating factor), BCMA, B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, carbonic anhydrase IX, cardiac myosin, CCL11 (C—C motif chemokine 11), CCR4 (C—C chemokine receptor type 4), CCR5, CD3E (epsilon), CEA (carcinoembryonic antigen), CEACAM3, CEACAM5 (carcino-embryonic antigen), CFD (Factor D), Ch4D5, cholecystokinin 2 (CCK2R), CLDN18 (Claudin-18), CLDN18.2 (Claudin-18.2), clumping factor A, cMet, CRIPTO, FCSF1R (colony stimulating factor 1 receptor), CSF2 (colony stimulating factor 2, granulocyte-macrophage colony-stimulating factor (GM-CSF)), CSP4, CTLA4 (cytotoxic T-lymphocyte-associated protein 4), CTAA16.88 tumor antigen, CXCR4, C—X—C chemokine receptor type 4, cyclic ADP ribose hydrolase, cyclin B1, CYP1B1, cytomegalovirus, cytomegalovirus glycoprotein B, Dabigatran, DLL3 (delta-like-ligand 3), DLL4 (delta-like-ligand 4), DPP4 (dipeptidyl-peptidase 4), DR5 (feath receptor 5), E. coli shiga toxin type-1, E. coli shiga toxin type-2, ED-B, EGFL7 (EGF-like domain-containing protein 7), EGFR, EGFRII, EGFRvIII, endoglin, endothelin B receptor, endotoxin, EpCAM (epithelial cell adhesion molecule), EphA2, Episialin, ERBB2 (epidermal growth factor receptor 2), ERBB3, ERG (TMPRSS2 ETS fusion gene), Escherichia coli, ETV6-AML, FAP (fibroblast activation protein alpha), FCGR1, alpha-Fetoprotein, Fibrin II, beta chain, fibronectin extra domain-B, FOLR (folate receptor), folate receptor alpha, folate hydrolase, Fos-related antigen 1F protein of respiratory syncytial virus, frizzled receptor, fucosyl GM1, GD2 ganglioside, G-28 (a cell surface antigen glyvolipid), GD3 idiotype, GloboH, glypican 3, N-glycolylneuraminic acid, GM3, GMCSF receptor α-chain, growth differentiation factor 8, GP100, GPNMB (trans-membrane glycoprotein NMB), GUCY2C (guanylate cyclase 2C, guanylyl cyclase C (GC-C), intestinal fuanylate cyclase, fuanylate cyclase-C receptor, heat-stable enterotoxin receptor (hSTAR)), heat shock proteins, hemagglutinin, hepatitis B surface antigen, hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, histone complex, HLA-DR (human leukocyte antigen), HLA-DR10, HLA-DRB, HMWMAA, human chorionic gonadotropin, HNGF, human scatter factor receptor kinase, HPV E6/E7, Hsp90, hTERT, ICAM-1 (Intercellular Adhesion Molecule 1), idiotype, IGF1R (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (insulin-like growth factor 2), Integrins (α4, α_{IIb}β₃, αvβ3, α₄β₇, α5β1, α6β4, α7β7, αIIβ3, α5β5, αvβ5), interferon gamma-induced protein, ITGA2, ITGB2, KIR2D, Kappa Ig, LCK, Le, Legumain, Lewis-Y antigen, LFA-1 (lymphocyte function-associated antigen 1, CD11a), LHRH, LINGO-1, lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (macrophage migration inhibitory factor, or glycosylation-inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, myelin-associated glycoprotein, myostatin, NA17, NARP-1, NCA-90 (granulocyte antigen), Nectin-4 (ASG-22ME), NGF, neural apoptosis-regulated proteinase 1, NOGO-A, Notch receptor, nucleolin, neu oncogene product, NY-BR-1, NY-ESO-1, OX-40, OxLDL (oxidized low-density lipoprotein), OY-TES1, P21, p53 nonmutant, P97, Page4, PAP, paratope of anti-(N-glycolylneuraminic acid), PAX3, PAX5, PCSK9, PDCD1 (PD-1, programmed cell death protein 1), PDGF-Rα (Alpha-type platelet-derived growth factor receptor), PDGFR-β, PDL-1, PLAC1, PLAP-like testicular alkaline phosphatase, platelet-derived growth factor receptor beta, phosphate-sodium co-transporter, PMEL 17, polysialic acid, proteinase3 (PR1), prostatic carcinoma, PS (Phosphatidylserine), prostatic carcinoma cells, Pseudomonas aeruginosa, PSMA, PSA, PSCA, rabies virus glycoprotein, RHD (Rh polypeptide 1 (RhPI)), Rhesus factor, RANKL, RhoC, Ras mutant, RGS5, ROBO4, respiratory syncytial virus, RON, ROR1, Sarcoma translocation breakpoints, SART3, sclerostin, SLAMF7 (SLAM family member 7), Selectin P, SDC1 (Syndecan 1), sLe(a), Somatomedin C, SIP (Sphingosine-1-phosphate), Somatostatin, sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (transforming growth factor beta), TGF-β1, TGF-β2 (transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF-13B (tumor necrosis factor receptor superfamily member 13B), TPBG (trophoblast glycoprotein), TRAIL-R1 (tumor necrosis apoptosis inducing ligand receptor 1), TRAILR2 (death receptor 5 (DR5)), tumor-associated calcium signal transducer 2, tumor specific glycosylation of MUC1, TWEAK receptor, TYRP1 (glycoprotein 75), TRP-1 (Trop-1), TRP-2 (Trop-2), tyrosinase, VCAM-1, VEGF, VEGF-A, VEGF-2, VEGFR-1, VEGFR2, or vimentin, WT1, XAGE 1, or a cell expressing an insulin growth factor receptor, or an epidermal growth factor receptor.

37. The conjugate according to claim 36, wherein the tumor cell is selected from the group consisting of lymphoma cells, myeloma cells, renal cells, breast cancer cells, prostate cancer cells, ovarian cancer cells, colorectal cancer cells, gastric cancer cells, squamous cancer cells, small-cell lung cancer cells, none small-cell lung cancer cells, testicular cancer cells, malignant cells, and cells that grow and divide at an unregulated, quickened pace to cause cancers.

38. A pharmaceutical composition comprising a therapeutically effective amount of at least one of the conjugate according to claim 22, and a pharmaceutically acceptable salt, carrier, diluent, or excipient therefore, for the treatment or prevention of a cancer, or an autoimmune disease, or an infectious disease.

39. The conjugate of claim 22, having in vitro, in vivo or ex vivo cell killing activity.

40. The pharmaceutical composition according to claim 38, further comprising a chemotherapeutic agent, a radiation therapy, an immunotherapy agent, an autoimmune disorder agent, an anti-infectious agent or a conjugate for synergistically treatment or prevention of a cancer, or an autoimmune disease, or an infectious disease.

41. The pharmaceutical composition according to claim 40, wherein the chemotherapeutic agent is one or more of:
(1). a). an alkylating agent: selected from nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; nitrosoureas: comprising carmustine, lomustine, chlorozotocin, fotemustine, nimustine, ranimustine; alkylsulphonates: including busulfan, treosulfan, improsulfan and piposulfan); triazenes or dacarbazine; platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine;
b). a plant alkaloid: selected from the group consisting of vinca alkaloids: including vincristine, vinblastine, vindesine, vinorelbine, and navelbin; taxoids: comprising paclitaxel, docetaxol and their analogs, maytansinoids including DM1, DM2, DM3, DM4, DM5, DM6, DM7, maytansine, ansamitocins and their analogs, cryptophycins (including the group of cryptophycin 1 and cryptophycin 8); epothilones, eleutherobin, discodermolide, bryostatins, dolostatins, auristatins, tubulysins, cephalostatins; pancratistatin; a sarcodictyin; spongistatin;
c). a DNA topoisomerase inhibitor: selected from the groups of epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d). an antimetabolite: selected from the group consisting of [Anti-folate: (DHFR inhibitors: comprising methotrexate, trimetrexate, denopterin, pteropterin, aminopterin (4-aminopteroic acid) or folic acid analogues); IMP dehydrogenase inhibitors: (including mycophenolic acid, tiazofurin, ribavirin, EICAR); ribonucleotide reductase inhibitors (including hydroxyurea, deferoxamine)]; [pyrimidine analogs: uracil analogs (including ancitabine, azacitidine, 6-azauridine, capecitabine, carmofur, cytarabine, dideoxyuridine, doxifluridine, enocitabine, 5-Fluorouracil, floxuridine, ratitrexed); cytosine analogs (including cytarabine, cytosine arabinoside, fludarabine); purine analogs (including azathioprine, fludarabine, mercaptopurine, thiamiprine, thioguanine)]; folic acid replenisher, frolinic acid;
e). a hormonal therapy: selected from receptor antagonists [anti-estrogen (including megestrol, raloxifene, tamoxifen); LHRH agonists (including goserelin, leuprolide acetate); anti-androgens (including bicalutamide, flutamide, calusterone, dromostanolone propionate, epitiostanol, goserelin, leuprolide, mepitiostane, nilutamide, testolactone, trilostane and other androgens inhibitors)]; retinoids/deltoids [vitamin D3 analogs (including CB 1093, EB 1089 KH 1060, cholecalciferol, ergocalciferol); photodynamic therapies (including verteporfin, phthalocyanine, photosensitizer Pc4, demethoxyhypocrellin A); cytokines (comprising interferon-alpha, interferon-gamma, tumor necrosis factor (TNFs), human proteins containing a TNF domain)];
f). a kinase inhibitor, selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib, bafetinib, bosutinib, cabozantinib, vismodegib, iniparib, ruxolitinib, CYT387, axitinib, tivozanib, sorafenib, bevacizumab, cetuximab, trastuzumab, ranibizumab, panitumumab, ispinesib;
g). a poly (ADP-ribose) polymerase (PARP) inhibitors selected from the group of olaparib, niraparib, iniparib, talazoparib, veliparib, CEP 9722 (Cephalon's), E7016 (Eisai's), BGB-290 (BeiGene's), or 3-aminobenzamide.
h). an antibiotic, selected from the group consisting of an enediyne antibiotic (selected from the group of calicheamicin, calicheamicin γ1, δ1, α1 or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin;
i). a polyketide (acetogenin), bullatacin and bullatacinone; gemcitabine, epoxomicins and carfilzomib, bortezomib, thalidomide, lenalidomide, pomalidomide, tosedostat, zybrestat, PLX4032, STA-9090, Stimuvax, allovectin-7, Xegeva, Provenge, Yervoy, isoprenylation inhibitors and lovastatin, dopaminergic neurotoxins and 1-methyl-4-phenylpyridinium ion, cell cycle inhibitors (including staurosporine), actinomycins (including actinomycin D, dactinomycin), amanitins, bleomycins (including bleomycin A2, bleomycin B2, peplomycin), anthracyclines (including daunorubicin, doxorubicin (adriamycin), idarubicin, epirubicin, pirarubicin, zorubicin, mtoxantrone, MDR inhibitors or verapamil, Ca²⁺ ATPase inhibitors or thapsigargin, histone deacetylase inhibitors (including vorinostat, romidepsin, panobinostat, valproic acid, mocetinostat (MGCD0103), belinostat, PCI-24781, entinostat, SB939, resminostat, givinostat, AR-42, CUDC-101, sulforaphane, trichostatin A); thapsigargin, celecoxib, glitazones, epigallocatechin gallate, disulfiram, salinosporamide A; anti-adrenals, selected from the group of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfornithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (including the group of T-2 toxin, verrucarin A, roridin A and anguidine); urethane, siRNA, antisense drugs;
(2). an anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus;
(3). an anti-infectious disease agents comprising of:
a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b). Amphenicols: azidamfenicol, chloramphenicol, florfenicol, thiamphenicol;
c). Ansamycins: geldanamycin, herbimycin;
d). Carbapenems: biapenem, doripenem, ertapenem, imipenem, cilastatin, meropenem, panipenem;
e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (including cefoxitin, cefotetan, cefmetazole), oxacephem (flomoxef, latamoxef);
f). Glycopeptides: bleomycin, vancomycin (including oritavancin, telavancin), teicoplanin (dalbavancin), ramoplanin;
g). Glycylcyclines: tigecycline;
h). p-Lactamase inhibitors: penam (sulbactam, tazobactam), clavam (clavulanic acid);
i). Lincosamides: clindamycin, lincomycin;
j). Lipopeptides: daptomycin, A54145, calcium-dependent antibiotics (CDA);
k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l). Monobactams: aztreonam, tigemonam;
m). Oxazolidinones: linezolid;
n). Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o). Polypeptides: bacitracin, colistin, polymyxin B;
p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q). Streptogramins: pristinamycin, quinupristin/dalfopristin;
r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s). Steroid antibacterials: selected from fusidic acid;
t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u). Other antibiotics: selected from the group consisting of annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4). anti-viral drugs of:
a). Entry/fusion inhibitors: aplaviroc, maraviroc, vicriviroc, gp41 (enfuvirtide), PRO 140, CD4 (ibalizumab);
b). Integrase inhibitors: raltegravir, elvitegravir, globoidnan A;
c). Maturation inhibitors: bevirimat, vivecon;
d). Neuraminidase inhibitors: oseltamivir, zanamivir, peramivir;
e). Nucleosides & nucleotides: abacavir, aciclovir, adefovir, amdoxovir, apricitabine, brivudine, cidofovir, clevudine, dexelvucitabine, didanosine (ddl), elvucitabine, emtricitabine (FTC), entecavir, famciclovir, fluorouracil (5-FU), 3′-fluoro-substituted 2′,3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), I-nucleosides (including the group consisting of, β-I-thymidine and β-I-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h). anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib;
(5). pharmaceutically acceptable salts, acids, derivatives, hydrate or hydrated salt; or a crystalline structure; or an optical isomer, racemate, diastereomer or enantiomer of any of the above drugs.

42. The conjugate according to claim 40, wherein the synergistic agent is one or more of following drugs: Abatacept, Abemaciclib, Abiraterone acetate, Abraxane, Acetaminophen/hydrocodone, Acalabrutinib, Aducanumab, Adalimumab, ADXS31-142, ADXS-HER2, Afatinib dimaleate, Aldesleukin, Alectinib, Alemtuzumab, Alitretinoin, ado-Trastuzumab emtansine, Amphetamine/dextroamphetamine, Anastrozole, Aripiprazole, Anthracyclines, Aripiprazole, Atazanavir, Atezolizumab, Atorvastatin, Avelumab, Axicabtagene ciloleucel, Axitinib, Belinostat, BCG Live, Bevacizumab, Bexarotene, Blinatumomab, Bortezomib, Bosutinib, Brentuximab vedotin, Brigatinib, Budesonide, Budesonide/formoterol, Buprenorphine, Cabazitaxel, Cabozantinib, Capmatinib, Capecitabine, Carfilzomib, chimeric antigen receptor-engineered T (CAR-T) cells, Celecoxib, Ceritinib, Cetuximab, Chidamide, Ciclosporin, Cinacalcet, Crizotinib, Cobimetinib, Cosentyx, CTL019, Dabigatran, Dabrafenib, Dacarbazine, Daclizumab, Dacomotinib, Daptomycin, Daratumumab, Darbepoetin alfa, Darunavir, Dasatinib, Denileukin diftitox, Denosumab, Depakote, Dexlansoprazole, Dexmethylphenidate, Dexamethasone, Dinutuximab, Doxycycline, Duloxetine, Duvelisib, Durvalumab, Elotuzumab, Emtricibine/Rilpivirine/Tenofovir, Disoproxil fumarate, Emtricitbine/Tenofovir/Efavirenz, Enoxaparin, ensartinib, Enzalutamide, Epoetin alfa, Erlotinib, Esomeprazole, Eszopiclone, Etanercept, Everolimus, Exemestane, Everolimus, Exenatide ER, Ezetimibe, Ezetimibe/simvastatin, Fenofibrate, Filgrastim, Fingolimod, Fluticasone propionate, Fluticasone/salmeterol, Fulvestrant, Gazyva, Gefitinib, Glatiramer, Goserelin acetate, Icotinib, Imatinib, Ibritumomab tiuxetan, Ibrutinib, Idelalisib, Ifosfamide, Infliximab, Imiquimod, ImmuCyst, Immuno BCG, Iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, lapatinib, Ipilimumab, Ipratropium bromide/salbutamol, Ixazomib, Kanuma, Lanreotide acetate, Lenalidomide, Lenaliomide, Lenvatinib mesylate, Letrozole, Levothyroxine, Levothyroxine, Lidocaine, Linezolid, Liraglutide, Lisdexamfetamine, LN-144, Lorlatinib, Memantine, Methylphenidate, Metoprolol, Mekinist, Mericitabine/Rilpivirine/Tenofovir, Modafinil, Mometasone, Mycidac-C, Necitumumab, Neratinib, Nilotinib, Niraparib, Nivolumab, Ofatumumab, Obinutuzumab, Olaparib, Olmesartan, Olmesartan/hydrochlorothiazide, Omalizumab, Omega-3 fatty acid ethyl esters, Oncorine, Oseltamivir, Osimertinib, Oxycodone, palbociclib, Palivizumab, Panitumumab, Panobinostat, Pazopanib, Pembrolizumab, PD-1 antibody, PD-L1 antibody, Pemetrexed, Pertuzumab, Pneumococcal conjugate vaccine, Pomalidomide, Pregabalin, ProscaVax, Propranolol, Quetiapine, Rabeprazole, Radium 223 chloride, Raloxifene, Raltegravir, Ramucirumab, Ranibizumab, Regorafenib, Ribociclib, Rituximab, Rivaroxaban, Romidepsin, Rosuvastatin, Ruxolitinib phosphate, Salbutamol, Savolitinib, Semaglutide, Sevelamer, Sildenafil, Siltuximab, Sipuleucel-T, Sitagliptin, Sitagliptin/metformin, Solifenacin, Solanezumab, Sonidegib, Sorafenib, Sunitinib, Tacrolimus, Tacrimus, Tadalafil, Tamoxifen, Tafinlar, Talimogene laherparepvec, Talazoparib, Telaprevir, Talazoparib, Temozolomide, Temsirolimus, Tenofovir/emtricitabine, Tenofovir disoproxil fumarate, Testosterone gel, Thalidomide, TICE BCG, Tiotropium bromide, Tisagenlecleucel, Toremifene, Trametinib, Trastuzumab, Trabectedin (ecteinascidin 743), Trametinib, Tremelimumab, Trifluridine/tipiracil, Tretinoin, Uro-BCG, Ustekinumab, Valsartan, Veliparib, Vandetanib, Vemurafenib, Venetoclax, Vorinostat, Ziv-aflibercept, Zostavax, and analogs, derivatives, pharmaceutically acceptable salts thereof.

43. The conjugate according to claim 22, wherein L has one of following structures:
—(CR₁₅R₁₆)ₘ(Aa)ᵣ(CR₁₇R₁₈)ₙ(OCH₂CH₂)ₜ, —(CR₁₅R₁₆)ₘ(CR₁₇R₁₈)ₙ(Aa)ᵣ(OCH₂CH₂)ₜ—, -(Aa)ᵣ (CR₁₅R₁₆)ₘ(CR₁₇R₁₈)ₙ(OCH₂CH₂)ᵣ, —(CR₁₅R₁₆)ₘ(CR₁₇R₁₈)ₙ(OCH₂CH₂)ᵣ(Aa)ᵣ, —(CR₁₅R₁₆)ₘ—(CR₁₇═CR₁₈)(CR₁₉R₂₀)ₙ(Aa)ₜ(OCH₂CH₂)ᵣ, —(CR₁₅R₁₆)ₘ(NR₁₁CO)(Aa)ₜ(CR₁₉R₂₀)ₘ(OCH₂CH₂)ᵣ, —(CR₁₅R₁₆)ₘ(Aa)ₜ(NR₂₁CO)(CR₁₉R₂₀)ₙ(OCH₂CH₂)ᵣ, —(CR₁₅R₁₆)ₘ(OCO)(Aa)ₜ(CR₁₉R₂₀)ₙ(OCH₂—CH₂)ᵣ, —(CR₁₅R₁₆)ₘ(OCNR₁₇)(Aa)ₜ(CR₁₉R₂₀)ₙ(OCH₂CH₂)ᵣ, —(CR₁₅R₁₆)ₘ—(CO)(Aa)ₜ(CR₁₉R₂₀)ₙ—(OCH₂CH₂)ᵣ, —(CR₁₅R₁₆)ₘ(NR₂₁CO)(Aa)ₜ(CR₁₉R₂₀)ₙ(OCH₂CH₂)ᵣ, —(CR₁₅R₁₆)ₘ(OCO)(Aa)ᵣ(CR₁₉R₂₀)ₙ—(OCH₂CH₂)ᵣ—, —(CR₁₅R₁₆)ₘ(OCNR₁₇)(Aa)ₜ(CR₁₉R₂₀)ₙ—(OCH₂CH₂)ᵣ, —(CR₁₅R₁₆)ₘ—(CO)(Aa)ₜ(CR₁₉R₂₀)ₙ—(OCH₂CH₂)ᵣ, —(CR₁₅R₁₆)ₘ-phenyl-CO(Aa)ₜ-(CR₁₇R₁₈)ₙ—, —(CR₁₅R₁₆)ₘ-furyl-CO(Aa)ₜ(CR₁₇R₁₈)ₙ—, —(CR₁₅R₁₆)ₘ-oxazolyl-CO(Aa)ₜ(CR₁₇R₁₈)ₙ—, —(CR₁₅R₁₆)ₘthiazolyl-CO-(Aa)ₜ(CCR₁₇R₁₈)ₙ—, —(CR₁₅R₁₆)ₜ-thienyl-CO(CR₁₇R₁₈)ₙ—, —(CR₁₅R₁₆)ᵣ-imidazolyl-CO—(CR₁₇R₁₈)ₙ—, —(CR₁₅R₁₆)ₜ-morpholino-CO(Aa)ₜ(CR₁₇R₁₈)ₙ—, —(CR₁₅R₁₆)ᵣ-piperazino-CO(Aa)ₜ-(CR₁₇R₁₈)ₙ—, —(CR₁₅R₁₆)ₜN-methylpiperazin-CO(Aa)ₜ(CR₁₇R₁₈)ₙ—, —(CR₁₅R₁₆)ₘ-(Aa)ₜphenyl-, —(CR₁₅R₁₆)ₘ-(Aa)ₜfuryl-, —(CR₁₅R₁₆)ₘ-oxazolyl(Aa)ₜ-, —(CR₁₅R₁₆)ₘ-thiazolyl(Aa)-, —(CR₁₅R₁₆)ₘ-thienyl-(Aa)-, —(CR₁₅R₁₆)ₘ-imidazolyl(Aa)ₜ-, —(CR₁₅R₁₆)ₘ-morpholino-(Aa)ᵣ—, —(CR₁₅R₁₆)ₙ-piperazino-(Aa)ₜ-, —(CR₁₅R₁₆)ₘ—N-methylpiperazino-(Aa)ₜ-, —K(CR₁₅R₁₆)ₘ(Aa)ᵣ(CR₁₇R₁₈)ₙ—(OCH₂CH₂)ₜ—, —K(CR₁₅R₁₆)ₘ—(CR₁₇R₁₈)ₙ(Aa)ᵣ(OCH₂CH₂)ₜ—, —K(Aa)ᵣ(CR₁₅R₁₆)ₘ(CR₁₇R₁₈)ₙ—(OCH₂CH₂)ₜ—, —K(CR₁₅R₁₆)ₘ—(CR₁₇R₁₈)ₙ(OCH₂CH₂)ᵣ(Aa)ᵣ, —K(CR₁₅R₁₆)ₘ(CR₁₇═CR₁₈)(CR₁₉R₂₀)ₙ(Aa)ₜ(OCH₂CH₂)ᵣ, —K(CR₁₅R₁₆)ₘ(NR₁₁CO)(Aa)ₜ-(CR₁₉R₂₀)ₙ(OCH₂CH₂)ᵣ, —K(CR₅R₆)ₘ(Aa)ᵣ(NR₂₁CO)(CR₁₉R₂₀)ₙ—(OCH₂CH₂)ᵣ, —K(CR₁₅R₁₆)ₘ(OCO)(Aa)ₜ(CR₁₉R₂₀)ₙ—(OCH₂CH₂)ᵣ, —K(CR₁₅R₁₆)ₘ(OCNR₁₇)(Aa)ₜ-(CR₁₉R₂₀)ₙ—(OCH₂CH₂)ᵣ, —K(CR₁₅R₁₆)ₘ(CO)(Aa)ₜ(CR₁₉R₂₀)ₙ(OCH₂—CH₂)ᵣ, —K(CR₁₅R₁₆)ₘ(NR₂₁—CO)(Aa)ₜ(CR₁₉R₂₀)ₙ—(OCH₂CH₂)ᵣ, —K(CR₁₅R₁₆)ₘ(OCO)(Aa)ᵣ(CR₁₉R₂₀)ₙ(OCH₂CH₂)ᵣ, —K—(CR₁₅R₁₆)ₘ—(OCNR₁₇)(Aa)ₜ-(CR₁₉R₂₀)ₙ(OCH₂CH₂)ᵣ, —K(CR₁₅R₁₆)ₘ—(CO)(Aa)ₜ(CR₁₉R₂₀)ᵣ—(OCH₂CH₂)ᵣ, —K(CR₁₅R₁₆)ₘ-phenyl-CO(Aa)ₜ(CR₁₇R₁₈)ₙ—, —K—(CR₁₅R₁₆)ₘ-furyl-CO(Aa)ₜ(CR₁₇R₁₈)ₙ, —K(CR₁₅R₁₆)ₘ-oxazolyl-CO(Aa)ₜ(CR₁₇R₁₈)ₙ—, —K(CR₁₅R₁₆)ₘ-thiazolyl-CO(Aa)ₜ(CR₁₇R₁₈)ₙ, —K(CR₁₅R₁₆)ₜ-thienyl-CO(CR₁₇R₁₈)ₙ—, —K(CR₁₅R₁₆)ₜimidazolyl-CO—(CR₁₇R₁₈)ₙ, —K(CR₅R₆)ₜmorpholino-CO(Aa)ₜ-(CR₁₇R₁₈)ₙ—, —K(CR₁₅R₁₆)ᵣ-piperazino-CO(Aa)ₜ-(CR₁₇R₁₈)ₙ—, —K(CR₁₅R₁₆)ₜN-methyl-piperazin-CO(Aa)ₜ(CR₁₇R₁₈)ₙ—, —K(CR₁₅R₁₆)ₘ(Aa)ₜ-phenyl, —K—(CR₁₅R₁₆)ₘ(Aa)ₜfuryl-, —K(CR₁₅R₁₆)ₘ-oxazolyl-(Aa)ₜ-, —K(CR₁₅R₁₆)ₘthiazolyl(Aa)ₜ, —K(CR₁₅R₁₆)ₘ-thienyl-(Aa)ₜ, —K(CR₁₅R₁₆)ₘ-imidazolyl(Aa)ᵣ, —K(CR₁₅R₁₆)ₘ-morpholino(Aa)ₜ, —K(CR₁₅R₁₆)ₘpiperazino(Aa)ₜG, —K(CR₅R₆)ₘ—N-methyl-piperazino(Aa)ₜ-,
wherein Aa, m, n, are described above; t and r here are 0-100 independently; R₁₃, R₁₄, R₁₅, R₁₆, R₁₇, R₁₈, R₁₉, R₂₀, and R₂₁ are independently H; halide; C₁-C₈ alkyl or heteroalkyl, C₂-C₈ aryl, alkenyl, alkynyl, ether, ester, amine or amide, or C₃-C₈ aryl, which optionally substituted by one or more halide, CN, NR₁₂R_{12′}, CF₃, OR₁₂, aryl, heterocycle, S(O)R₁₂, SO₂R₁₂, —CO₂H, —SO₃H, —OR₁₂, —CO₂R₁₂, —CONR₁₂, —PO₂R₁₂R₁₃, —PO₃H or P(O)R₁₂R_{12′}R₁₃; K is NH, NR₁₂, —SS—, —C(═O)—, —C(═O)NH—, —C(═O)O—, —C═NH—O—, —C═N—NH—, —C(═O)NH—NH—, O, S, Se, B, Het (heterocyclic or heteroaromatic ring having C₃-C₁₂); or a peptide containing the same or different 1-20 amino acids.

44. The conjugate according to claim 28, wherein the amino acid in the linker L is an aspartic acid, a glutamic acid, a lysine, an ornithine, or a tyrosine wherein one or two of their functional amino group, carboxylic group or phenol group are linked to the long side chain of the formula (I-q).

45. The process according to claim 30, wherein the reacting group is selected from a halide (fluoride, chloride, bromide, and iodide), maleimide, methanesulfonyl (mesyl), toluenesulfonyl (tosyl), trifluoromethyl-sulfonyl (triflate), trifluoro-methylsulfonate, nitrophenoxyl, N-succinimidyloxyl (NHS), phenoxyl; dinitrophenoxyl; pentafluorophenoxyl, tetrafluorophenoxyl, trifluorophenoxyl, difluoro-phenoxyl, monofluoro-phenoxyl, pentachloro-phenoxyl, 1H-imidazole-1-yl, chlorophenoxyl, dichloro-phenoxyl, trichlorophenoxyl, tetrachlorophenoxyl, N-(enzotriazole-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphrus, sulfur-nitrogen, phosphrus-nitrogen, oxygen-nitrogen, or carbon-oxygen), or an intermediate molecule generated with a condensation reagent for Mitsunobu reactions, the condensation reagent is: EDC (N-(3-dimethyl-aminopropyl)-N′-ethylcarbodiimide), DCC (dicyclohexyl-carbodiimide), N,N′-diisopropyl-carbodiimide (DIC), N-cyclohexyl-N′-(2-morpholinoethyl)-carbodiimide metho-p-toluenesulfonate (CMC, or CME-CDI), 1,1′-carbonyldiimidazole (CDI), TBTU (O-(benzotriazol-1-yl)-N,N,N′,N′-tetra-methyluronium tetrafluoroborate), N,N,N′,N′-tetramethyl-O-(1H-benzotriazol-1-yl)-uronium hexafluoro-phosphate (HBTU), (benzotriazol-1-yloxy)tris-(dimethylamino)-phosphonium hexafluorophosphate (BOP), (benzotriazol-1-yloxy)-tripyrroli-dinophosphonium hexafluorophosphate (PyBOP), diethyl cyanophosphonate (DEPC), chloro-N,N,N′,N′-tetramethylformamidiniumhexafluorophosphate, 1-[bis(dimethylamino)-methylene]-1H-1,2,3-triazolo[4,5-b]pyridinium 3-oxid hexafluorophos-phate (HATU), 1-[(di-methylamino)(morpholino)methylene]-1H-[1,2,3]triazolo[4,5-b]pyridine-1-ium 3-oxide hexafluoro-phosphate (HDMA), 2-chloro-1,3-dimethyl-imidazolidinium hexafluorophosphate (CIP), chlorotripyrrolidinophosphonium hexafluorophosphate (PyCloP), fluoro-N,N,N′,N′-bis(tetramethylene)-formamidinium hexafluorophosphate (BTFFH), N,N,N′,N′-tetramethyl-S-(1-oxido-2-pyridyl)-thiuronium hexafluorophosphate, O-(2-oxo-1(2H)pyridyl)-N,N,N′,N′-tetramethyluronium tetrafluoroborate (TPTU), S-(1-oxido-2-pyridyl)-N,N,N′,N′-tetramethyl-thiuronium tetrafluoroborate, O-[(ethoxycarbonyl)-cyanomethylenamino]-N,N,N′,N′-tetramethyluronium hexafluorophosphate (HOTU), (1-cyano-2-ethoxy-2-oxoethylidenamino-oxy)dimethylamino-morpholino-carbenium hexafluorophosphate (COMU), O-(benzotriazol-1-yl)-N,N,N′,N′-bis(tetramethylene)enzotr hexafluorophosphate (HBPyU), N-benzyl-N′-cyclohexyl-carbodiimide (with, or without polymer-bound), dipyrrolidino(N-succinimidyl-oxy)carbenium hexafluoro-phosphate (HSPyU), chlorodipyrrolidinocarbenium hexafluoro-phosphate (PyClU), 2-chloro-1,3-dimethylimidazolidinium tetrafluoroborate (CIB), (benzotriazol-1-yloxy)-dipiperidino-carbenium hexafluorophosphate (HBPipU), O-(6-chlorobenzotriazol-1-yl)-N,N,N′,N′-tetramethyluronium tetrafluoroborate (TCTU), bromotris(dimethylamino)-phosphonium hexafluoro-phosphate (BroP), propylphosphonic anhydride (PPACA, T3P), 2-morpholinoethyl isocyanide (MEI), N,N,N′,N′-tetramethyl-O—(N-succinimidyl)-uronium hexafluorophosphate (HSTU), 2-bromo-1-ethyl-pyridinium tetrafluoro-borate (BEP), O-[(ethoxycarbonyl)cyano-methylenamino]-N,N,N′,N′-tetra-methyluronium tetrafluoroborate (TOTU), 4-(4,6-dimethoxy-1,3,5-triazin-2-yl)-4-methylmorpholinium chloride (MMTM, DMTMM), N,N,N′,N′-tetramethyl-O—(N-succinimidyl)enzotr tetrafluoroborate (TSTU), O-(3,4-dihydro-4-oxo-1,2,3-benzotriazin-3-yl)-N,N,N′,N′-tetramethyluronium tetrafluoro-borate (TDBTU), 1,1′-(azodicarbonyl)-dipiperidine (ADD), di-(4-chlorobenzyl)-azodicarboxylate (DCAD), di-tert-butyl azodicarboxylate (DBAD), diisopropyl azodicarboxylate (DIAD), diethyl azodicarboxylate (DEAD), or an anhydride formed by an acid itself or formed with other C₁-C₈ acid anhydride.

46. The process according to claim 30, wherein Lv is a halide (fluoride, chloride, bromide, and iodide), maleimide, methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(enzotriazole-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or carbon-oxygen), or one of following structures: 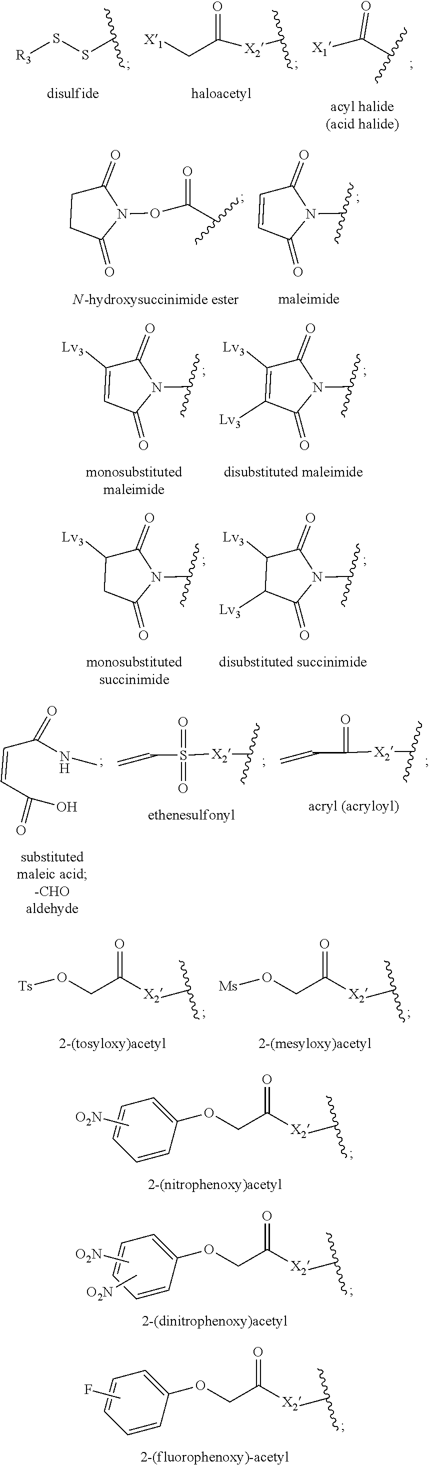 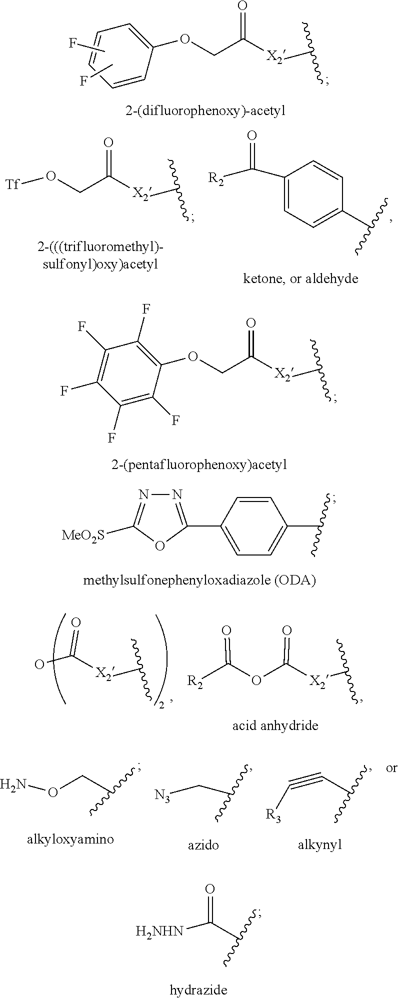
wherein X₁′ is F, Cl, Br, I or Lv₃; X₂′ is O, NH, N(R₁), or CH₂; R₃ is H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁, wherein R₁ and R₂ are defined the same as in claim 30; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzo-triazole; tosylate; mesylate; 2-ethyl-5-phenylisoxazolium-3′-sulfonate, anhydrides formed by themselves, or formed with acetyl anhydride or formyl anhydride.

47. The conjugate according to claim 31, wherein mAb is an antibody.
